SEQUENCE LISTING (3 sequences)

SEQ ID NO: 1 - DNA, 51 bp, Homo sapiens
tgtaagagcagatccctggacaggcraggaatacaggtattttgtccttga

SEQ ID NO: 2 - DNA, 50 bp, Homo sapiens
tgtcaaccagagtctcctgactcccctcaacctgcaaatcgaccccagca

SEQ ID NO: 3 - DNA, 50 bp, Homo sapiens
ccagagggtgaggaccgaggagcgcgagcagatcaagaccctcaacaata

(19) United States
(12) Patent Application Publication
(10) Pub. No.: US 2009/0307180 A1
(43) Pub. Date: Dec. 10, 2009

(54) Genetic analysis

(76) Inventors: Brandon Colby, Los Angeles, CA (US); Bethany Slater, Palo Alto, CA (US); Melvyn Colby, Great Neck, NY (US); Bryon Colby, New York, NY (US)

Correspondence Address: WILSON, SONSINI, GOODRICH & ROSATI, 650 PAGE MILL ROAD, PALO ALTO, CA, 94304-1050, US

(21) Appl. No.: 12/383,117

(22) Filed: Mar. 18, 2009

Related U.S. Application Data
(60) Provisional application No. 61/037,959, filed on Mar. 19, 2008.
(60) Provisional application No. 61/050,126, filed on May 2, 2008.
(60) Provisional application No. 61/091,342, filed on Aug. 22, 2008.
(60) Provisional application No. 61/136,266, filed on Aug. 22, 2008.
(60) Provisional application No. 61/198,765, filed on Nov. 7, 2008.

(51) Int. Cl. G06N 5/02; C12Q 1/68

(52) U.S. Cl. 706/54; 435/6

(57) ABSTRACT

The present invention provides methods for generating genetic profiles or analyses. Included are methods for conducting comprehensive, dynamic genetic analysis. Also provided are methods for determining genetic health scores for specific phenotypes, such as diseases, disorders, traits, and conditions, as well as for organ systems, for certain medical specialties, and for overall health.

### CROSS-REFERENCE

This application claims the benefit of U.S. Provisional Application No. 61/037,959 filed Mar. 19, 2008, U.S. Provisional Application No. 61/050,126 filed May 2, 2008, U.S. Provisional Application No. 61/091,342 filed Aug. 22, 2008, U.S. Provisional Application No. 61/136,266 filed Aug. 22, 2008, and U.S. Provisional Application No. 61/198,765 filed Nov. 7, 2008, all of which are incorporated herein by reference in their entirety. This application relates to U.S. patent application Ser. No. ______, entitled “Genetic Analysis,” Attorney Docket No. 35925-702.201; U.S. patent application Ser. No. ______, entitled “Genetic Analysis,” Attorney Docket No. 35925-702.202; and U.S. patent application Ser. No. ______, entitled “Genetic Analysis,” Attorney Docket No. 35925-702.204, all of which are concurrently filed in the U.S. Patent and Trademark Office on Mar. 18, 2009, and all of which are hereby incorporated herein by reference in their entirety. This application also relates to International Application No. ______, entitled “Genetic Analysis,” Attorney Docket No. 35925-702.601, which is concurrently filed in the U.S. Receiving Office on Mar. 18, 2009, and which is hereby incorporated herein by reference in its entirety.

### BACKGROUND

The genomes of organisms contain a vast amount of information that can be mined in order to predict, identify or describe observable characteristics of an organism, such as diseases, conditions, disorders, traits, characteristics, morphology, biochemical properties, or physiologic properties. Observable characteristics can also be affected, determined, or predicted from environmental conditions, or from some combination of genetic and environmental conditions. There is an unmet need for an intelligent approach to using genetic and non-genetic information to predict, identify, analyze or describe phenotypes in an organism.

### SUMMARY OF THE INVENTION

Provided herein is a method of determining the predisposition or carrier status of an individual for two or more phenotypes related to suitability for military service comprising: identifying by nucleic acid array, sequencing apparatus, or nanopore sequencer a set of genetic variants in an individual, wherein each of said genetic variants is correlated with a suitability for military service phenotype; using a computer to determine the predisposition or carrier status of said individual for at least two phenotypes, wherein said predisposition or carrier status is based on said set of genetic variants; providing a report of said predisposition or carrier status to said individual, to a health care provider of said individual, or to a third party; and optionally combining the predisposition or carrier status of said individual for said at least two phenotypes into a suitability for military service score, wherein said score is reported to said individual, to a health care provider, or to a third party.

In an embodiment, at least two phenotypes comprise an initial phenotype and a reflex phenotype, wherein said reflex phenotype is a phenotype that is not the initial phenotype, and wherein the reporting of the predisposition or carrier status of said individual for the reflex phenotype depends on the outcome of said determination of predisposition or carrier status of said individual for the initial phenotype. In some embodiments, at least two phenotypes are at least two phenotypes listed in one or more of the following figures: Military and Armed Forces Panel Alpha (FIG. 16), or Military and Armed Forces Panel Beta (FIG. 17). In other embodiments, at least two phenotypes comprises at least five phenotypes. In further embodiments, at least two phenotypes comprise: at least one phenotype that follows monogenic inheritance; and at least one phenotype that follows multifactorial or polygenic inheritance.

In another embodiment, at least two phenotypes comprises at least two of the following phenotypes: universal identifier; blood group; extreme high or low intelligence quotient; post traumatic stress disorder susceptibility; adverse reaction to smallpox vaccination; sensitivity to weapons of mass destruction; extreme high or low visual acuity; or athletic ability, or predisposition to specific sports. In yet another embodiment, at least two phenotypes further comprise at least one of the following phenotypes: thrombophilia or thromboembolic disease; psychiatric illness; personality traits; effect of stimulants on cognition; stressful life events causing depressive symptoms, diagnosable depression, suicidality or anxiety; infectious disease susceptibility.

In an embodiment, at least two phenotypes comprises at least two of the following phenotypes: universal identifier; post traumatic stress disorder susceptibility; specific physical exercise regimen for most efficient physical exercise; thrombophilia or thromboembolic disease. In some embodiments, at least two phenotypes further comprise at least one of the following phenotypes: violent behavior; noise-induced hearing impairment or hearing loss; effect of stimulants on cognition; stressful life events causing depressive symptoms, diagnosable depression, suicidality or anxiety; malaria susceptibility; arrhythmogenic right ventricular cardiomyopathy.

In an embodiment, said at least two phenotypes comprise an initial phenotype and a reflex phenotype, wherein said reflex phenotype is reported when said individual has an increased predisposition or carrier status for said initial phenotype. In some embodiments, said reflex phenotype is reported when said individual has a decreased predisposition or carrier status for said initial phenotype. In other embodiments, said reflex phenotype is not reported if the individual has neither a decreased nor increased predisposition nor carrier status for said initial phenotype. In further embodiments, said reflex phenotype is reported concurrently with said initial phenotype. In another embodiment, said reflex phenotype is reported subsequently to said initial phenotype. In an embodiment of the method, the determination of the predisposition or carrier status of the individual for said reflex phenotype is determined subsequently to the determination of the predisposition or carrier status of the individual for said initial phenotype. In yet another embodiment, said reflex phenotype is a disease that is positively correlated with said initial phenotype.

In an embodiment, said initial phenotype is a disease and said reflex phenotype is a symptom of said disease. In some embodiments, said initial phenotype is a disease or disorder and reflex phenotype is a side effect of, or response to, a treatment for said initial phenotype. In other embodiments, said initial phenotype is thrombophilia or a thromboembolic disorder, and said reflex phenotype is one or more selected from the group consisting of: warfarin suitability; and suitability of anti-thrombotic medications or NSAIDS.

In an embodiment of the method, wherein said at least two phenotypes comprise an initial phenotype and a reflex phenotype, said initial phenotype is a psychiatric illness, and said reflex phenotype is one or more selected from the group consisting of: treatment-emergent suicidality during treatment with antidepressents; suitability of medications used to treat depression; response rates to standard treatment for late-life depression; aggressiveness or homicidal behavior with schizophrenia; severity or symptomology of schizophrenia; suitability of mood stabilizers or antipsychotic medications; cognitive performance with bipolar disorder; antipsychotic medication induced parkinsonism; and lithium response in mania or bipolar disorder. In some embodiments, said initial phenotype is effect of stimulus on cognition, and said reflex phenotype is one or more selected from the group consisting of: stimulant induced adverse reactions, and drug addiction. In other embodiments, said initial phenotype is stressful life events causing depressive symptoms diagnosable depression or anxiety, and said reflex phenotype is one or more selected from the group consisting of: suitability of medications used to treat depression; treatment-emergent suicidality during treatment with antidepressants; and suitability of medication for treatment for anxiety.

In further embodiments, said initial phenotype is infectious disease susceptibility, and said reflex phenotype is one or more selected from the group consisting of: suitability of medication to treat HIV infection; prognosis, rate of progression, CD4 count or viral load with HIV infection; risk of HIV dementia; suitability of medications used to treat infections; severity or prognosis with HCV infection; suitability of medications used to treat hepatitis C virus infection; severity or prognosis with meningococcal disease; age at onset of prion diseases; hepatitis B virus infection prognosis or rate of hepatitis B virus clearance; vaccine-induced immunity to hepatitis B virus infection; glucose-6-phosphate dehydrogenase deficiency; severity, prognosis, mortality, morbidity or parasite load with malarial infection; suitability of medication used to treat malarial infection or for malarial prophylaxis; response to Lepromin; disease and prognosis following M. leprae infection; severity or prognosis of herpes simplex virus infection; and iron deficiency or iron deficiency anemia during malaria season. In another embodiment, said initial phenotype is malaria susceptibility, and said reflex phenotype is one or more selected from the group consisting of: glucose-6-phosphate dehydrogenase deficiency, severity, prognosis or parasite load with malarial infection; prognosis, mortality or severity with malarial infection; suitability of medication used to treat malarial infection or for malarial prophylaxis; and iron deficiency or iron deficiency anemia during malaria season. In yet another embodiment, said initial phenotype is arrhythmogenic right ventricular cardiomyopathy, and said reflex phenotype is one or more selected from the group consisting of: suitability of antiarrhythmogenic medication; and digoxin absorption, metabolism or toxicity.

In an embodiment, said initial phenotype is height or weight, and said reflex phenotype is one or more selected from the group consisting of: response of stature to human growth hormone; diabetes mellitus type II; amount of effort needed to lose weight; dyslipidemia or lipid levels with increased BMI or obesity; change in body fat or lipid levels with specific diets or with exercise; and exercise tolerance, optimal exercise regimen or athletic training regimen for weight management. In some embodiments, said initial phenotype is susceptibility to bacteremia, sepsis, severe sepsis, septic shock, or systemic inflammatory response syndrome, and said reflex phenotype is one or more selected from the group consisting of: severity of sepsis, septic shock, severe sepsis or systemic inflammatory response syndrome; source of infection, type of bacteria with bacteremia, sepsis, severe sepsis, septic shock or systemic inflammatory response syndrome. In other embodiments, said initial phenotype is meningococcal disease susceptibility and said reflex phenotypes is severity of meningococcal disease. In further embodiments, said initial phenotype is tuberculosis susceptibility and said reflex phenotypes is clinical manifestation of tuberculosis infection. In another embodiment, said initial phenotype is hypertrophic cardiomyopathy and said reflex phenotypes is heart wall thickness with cardiomyopathy.

In an embodiment of the method of determining the predisposition or carrier status of an individual for two or more phenotypes related to suitability for military service, said predisposition or carrier status is determined from at least two genetic variants. In some embodiments, said at least two genetic variants are correlated with the same phenotype. In other embodiments, said predisposition or carrier status is determined for adverse reaction to smallpox vaccination and at least one of said genetic variants is selected from the group consisting of, or in linkage disequilibrium with, at least one genetic variant selected from the group consisting of: rs839, rs1801133, and rs9282763. In further embodiments, said predisposition or carrier status is determined for universal identifier and at least one of said genetic variants is selected from the group consisting of, or in linkage disequilibrium with, at least one genetic variant selected from the group consisting of: rs8176747, rs8176741, rs6444724, rs1336071, rs7520386, ABO Chr. 9: 135122733 delG, rs1019029, rs2073383, rs13218440, rs1478829, rs3780962, rs214955, rs13134862, rs1410059, rs7205345, rs321198, rs338882, rs10488710, rs279844, rs6811238, rs1058083, rs13182883, rs8176749, rs560681, rs10092491, rs740598, rs445251, rs1358856, rs1821380, rs1523537, rs7229946, rs8176720, rs2567608, rs9951171, rs1554472, rs1109037, rs2272998, rs987640, rs 12997453, rs2503107, rs447818, rs7704770, rs315791, rs6591147, rs985492, rs8176743, and rs8176746. In yet another embodiment, said predisposition or carrier status is determined for blood group and at least one of said genetic variants is selected from the group consisting of, or in linkage disequilibrium with, at least one genetic variant selected from the group consisting of: rs8176741, rs12075, rs11276, rs8176720, rs8176743, rs8176747, SLC14A1 Chr. 18: 41573550 Y, ABO Chr. 9: 135121239 S, ABO Chr. 9: 135121469 Y, ABO Chr. 9: 135122733 delG, rs2285644, rs 1058396, rs5036, rs8176058, rs28399653, rs 1135062, rs3894326, rs28362459, rs28362692, and CD 151 Chr. 11: 827536 R.

In an embodiment, said predisposition or carrier status is determined for intelligence and at least one of said genetic variants is selected from the group consisting of, or in linkage disequilibrium with, at least one genetic variant selected from the group consisting of: rs8191992, rs2061174, rs363043, rs353016, rs58646131, rs4680, and rs1130233. In some embodiments, said predisposition or carrier status is determined for post traumatic stress disorder susceptibility and at least one of said genetic variants is selected from the group consisting of, or in linkage disequilibrium with, at least one genetic variant selected from the group consisting of: rs9296158, rs6277, rs4606, rs1360780, and rs9470080. In other embodiments, said predisposition or carrier status is determined for athletic ability or athletic predisposition and at least one of said genetic variants is selected from the group consisting of, or in linkage disequilibrium with, at least one genetic variant selected from the group consisting of: rs11689011, rs1815739, rs1867785, rs895436, rs4035887, rs4646994, rs17602729, MSTN Chr. 2: 190635190 R, MTCYB Mito: 15615 R, MTCYB Mito: 14846 R, MTCYB Mito: 15497 R, and MTTG Mito: 10010 Y. In further embodiments, said predisposition or carrier status is determined for thrombophilia or thromboembolic disease and at least one of said genetic variants is selected from the group consisting of, or in linkage disequilibrium with, at least one genetic variant selected from the group consisting of: rs6025, rs6046, rs5985, rs1801133, rs1800790, rs2232354, rs9574, rs5985, rs1800595, rs1799963, rs2232698, SERPINA10 Chr. 14: 93824396 R, PROC Chr. 2: 127900253 Y, PROC Chr. 2: 127895484 R, PROC Chr. 2: 127902541 Y, PROS1 Chr. 3: 95080840 Y, PROS1 Chr. 3: 95086439 R, F11 Chr. 4: 187429867 Y, F11 Chr. 4: 187438406 Y, FGA Chr. 4: 155730115 R, FGA Chr. 4: 155727040 R, THBD Chr. 20: 22976686 K, rs5907, FGB Chr. 4: 155706587 R, TFPI Chr. 2: 188057188 Y, PLG Chr. 6: 161079615 R, FGG Chr. 4: 155747369 W, SERPINC1 Chr. 1: 172150331 Y, and SERPINC1 Chr. 1: 172139799 S.

In an embodiment of the method of determining the predisposition or carrier status of an individual for two or more phenotypes related to suitability for military service, said individual selects said two or more phenotypes. In some embodiments, said set of genetic variants was identified using a high density DNA microarray. In other embodiments, said set of genetic variants was identified by sequencing genomic DNA from said individual. In further embodiments, said individual is a military trainee. In another embodiment, said individual is a member of the military. In yet another embodiment, said individual is a law enforcement officer.

In an embodiment of the method of determining the predisposition or carrier status of an individual for two or more phenotypes related to suitability for military service, the results of said determination are used to rank applicants for service in the military or a law enforcement agency. In some embodiments, said individual tests positive for a sensitivity or adverse reactions from small pox vaccination phenotype, said method further comprising disqualifying said individual from small pox vaccination or from duties likely to expose said individual to smallpox. In other embodiments, said individual tests positive for a psychiatric illness phenotype, said method further comprising monitoring said individual for signs of psychiatric illness. In further embodiments, said individual tests positive for a psychiatric illness phenotype, said method further comprising disqualifying said individual for service in the military or a law enforcement agency.

Another aspect provided herein is a suitability-for-military-service set of probes, wherein said set comprises probes, wherein each of said probes is specifically selected to detect a genetic variant correlated with a suitability-for-military-service-related safety phenotype. In an embodiment of the suitability-for-military-service set of probes, said set detects at least two phenotypes listed in the following figures: Military and Armed Forces Panel Alpha (FIG. 16), or Military and Armed Forces Panel Beta (FIG. 17). In an embodiment of the suitability-for-military-service set of probes, said set comprises at least two probes, and each of said at least two probes detects a different genetic variant, and wherein each of said different genetic variants is correlated to the same phenotype.

A further aspect provided herein is a method of determining the predisposition or carrier status of an individual for two or more Law Enforcement phenotypes related to comprising: identifying by nucleic acid array, sequencing apparatus, or nanopore sequencer a set of genetic variants in an individual, wherein each of said genetic variants is correlated with a Law Enforcement phenotype; using a computer to determine the predisposition or carrier status of said individual for at least two phenotypes, wherein said predisposition or carrier status is based on said set of genetic variants; providing a report of said predisposition or carrier status to said individual, to a health care provider of said individual, law enforcement official, forensic investigator, or to a third party; and, optionally, combining the predisposition or carrier status of said individual for said at least two phenotypes into a Law Enforcement score, wherein said score is reported to said individual, to a health care provider of said individual, or to a third party.

In an embodiment, at least two phenotypes comprise an initial phenotype and a reflex phenotype, wherein said reflex phenotype is a phenotype that is not the initial phenotype, and wherein the reporting of the predisposition or carrier status of said individual for the reflex phenotype depends on the outcome of said determination of predisposition or carrier status of said individual for the first phenotype. In some embodiments, at least two phenotypes are at least two phenotypes listed in the following figure: Law Enforcement Panel (FIG. 18). In other embodiments, at least two phenotypes comprises at least five phenotypes. In further embodiments, at least two phenotypes comprise: at least one phenotype that follows monogenic inheritance; and at least one phenotype that follows multifactorial or polygenic inheritance. In another embodiment, at least two phenotypes comprises at least two of the following phenotypes: universal identifier or identity testing; blood group; physical traits; linear and/or ancestry information; height and/or weight; personality traits; psychiatric illness; age; cardiac arrhythmia or cardiac conduction abnormality; hypertrophic cardiomyopathy; thrombophilia or thromboembolic disease; stressful life events; visual acuity; level of aggression in behavior/personality; tendency to experience unprovoked anger; and mental vulnerability to social stressors and chronic disease.

In yet another embodiment, said reflex phenotype is reported when said individual has an increased predisposition or carrier status for said initial phenotype. In an embodiment, said reflex phenotype is reported when said individual has a decreased predisposition or carrier status for said initial phenotype. In some embodiments, said reflex phenotype is not reported if the individual has neither a decreased nor increased predisposition nor carrier status for said initial phenotype. In some embodiments, said reflex phenotype is reported concurrently with said initial phenotype. In other embodiments, said reflex phenotype is reported subsequently to said initial phenotype. In other embodiments, the determination of the predisposition or carrier status of the individual for said reflex phenotype is determined subsequently to the determination of the predisposition or carrier status of the individual for said initial phenotype. In other embodiments, said reflex phenotype is a disease that is positively correlated with said initial phenotype. In further embodiments, said initial phenotype is a disease and said reflex phenotype is a symptom of said disease. In another embodiment, said initial phenotype is a disease or disorder and reflex phenotype is a side effect of, or response to, a treatment for said initial phenotype.

In yet another embodiment, said initial phenotype is height or weight, and said reflex phenotype is one or more selected from the group consisting of: response of stature to human growth hormone; diabetes mellitus, type II; amount of effort needed to lose weight; dyslipidemia and/or lipid levels with increased BMI and/or obesity; change in body fat and/or lipid levels with specific diets and/or with exercise; exercise tolerance and/or optimal exercise regimen and/or athletic training regimen for weight management. In an embodiment, said initial phenotype is psychiatric illness, and said reflex phenotype is one or more selected from the group consisting of: treatment-emergent suicidality during treatment with antidepressants; effectiveness and/or sensitivity and/or response to medications used to treat depression; response rates to standard treatment for late-life depression; aggressiveness or homicidal behavior with schizophrenia; severity or symptomology of schizophrenia; aggressiveness or homicidal behavior with schizophrenia; dose and/or choice and/or effectiveness and/or sensitivity and/or response and/or adverse reactions to mood stabilizers and/or antipsychotic medications; cognitive performance with bipolar disorder; antipsychotic medication induced parkinsonism; lithium response in mania and/or bipolar disorder. In some embodiments, said initial phenotype is cardiac arrhythmia or cardiac conduction abnormality, and said reflex phenotype is one or more selected from the group consisting of: drug-induced torsade de pointes; drug-induced long QT syndrome; suitability of antiarrhythmogenic medication; digoxin suitability; age of onset of atrial fibrillation; QTc length, severity, symptoms, and prognosis with long QT syndrome.

In other embodiments, said initial phenotype is hypertrophic cardiomyopathy, and said reflex phenotype is heart wall thickness with cardiomyopathy. In further embodiments, said initial phenotype is thrombophilia or a thromboembolic disorder, and said reflex phenotype is one or more selected from the group consisting of: warfarin suitability; and suitability of anti-thrombotic medications or NSAIDs. In another embodiment, said initial phenotype is depression or seasonal affective disorder and said reflex phenotype is one or more selected from the group consisting of: suitability of medications used to treat depression; treatment-emergent suicidality during treatment with antidepressants; response to treatment for depression; and suitability of medication for treatment of anxiety. In yet another embodiment, said initial phenotype is stressful life events causing depressive symptoms diagnosable depression or anxiety, and said reflex phenotype is one or more selected from the group consisting of: suitability of medications used to treat depression; treatment-emergent suicidality during treatment with antidepressants; and effectiveness and choice of medication for treatment for anxiety.

In an embodiment, said predisposition or carrier status is determined from at least two genetic variants. In some embodiments, at least two genetic variants are correlated with the same phenotype. In other embodiments, said predisposition or carrier status is determined for height or weight and at least one of said genetic variants is selected from the group consisting of, or in linkage disequilibrium with, at least one genetic variant selected from the group consisting of: rs6060369, rs6830062, rs1867138, rs724016, rs7846385, rs1492820, rs10946808, rs314277, rs4896582, rs2040494, rs9650315, rs1042725, rs8007661, rs2562784, rs12986413, rs6060369, rs6440003, rs2282978, rs6060373, rs1390401, rs3116602, rs6686842, rs10906982, rs7901695, rs6724465, rs10935120, rs8041863, rs4794665, rs757608, rs4800148, rs967417, rs16896068, rs4549631, rs3791675, rs2814993, rs10512248, rs12735613, rs11107116, rs6854783, rs8099594, rs11205277, rs678962, rs2274432, rs3791679, rs6763931, rs6842303, rs1812175, rs12198986, rs2844479, rs3130050, rs185819, rs1776897, rs4713858, rs3748069, rs798544, rs11765954, rs10498015, rs10958476, rs4743034, rs8756, rs7153027, rs4533267, rs3760318, rs324420, rs9930506, rs4740294, rs2241766, rs9939609, rs1801260, rs2293855, rs2272382, rs745229, rs4129733, rs17782313, rs1528133, rs7799039, rs1801282, rs7566605, rs4632532, rs7561317, rs182052, rs1042713, rs2889849, rs10498015, rs1421085, rs1528133, rs7034356, rs8050136, rs1455832, rs2774279, rs968671, rs2241766, rs4818, rs7138803, and rs4680.

In further embodiments, said predisposition or carrier status is determined for suicidality and at least one of said genetic variants is selected from the group consisting of, or in linkage disequilibrium with, at least one genetic variant selected from the group consisting of: rs1386494, rs25531, rs12936511, rs6265, rs4792887, and rs4675690. In another embodiment, said predisposition or carrier status is determined for bipolar disorder and at least one of said genetic variants is selected from the group consisting of, or in linkage disequilibrium with, at least one genetic variant selected from the group consisting of: rs1298865, rs942518, rs12899449, rs17110563, rs25531, rs1006737, rs12899449, rs41261045, rs10994336, rs4511, rs9462082, rs4680, rs6265, rs2230912, rs133845, ADRBK2 Chr. 22: 24290897 delG, rs6986303, rs138784, rs1170191, rs821633, rs11089599, rs1344706, rs11568190, rs2391191, rs3918346, rs2637777, rs7680321, rs2304865, rs4979416, rs10994336, rs1485171, rs12899449, and rs10937823. In yet another embodiment, said predisposition or carrier status is determined for atrial fibrillation and at least one of said genetic variants is selected from the group consisting of, or in linkage disequilibrium with, at least one genetic variant selected from the group consisting of: KCNJ2 Chr. 17: 65683052 R, rs2200733, rs10033464, rs13143308, KCNJ1 Chr. 17: 65683052 R, KCNQ1 Chr. 11: 2505765 R, KCNQ1 Chr. 11: 2505768 R, and KCNE2 Chr. 21: 34664726 Y.

In an embodiment, said predisposition or carrier status is determined for hypertrophic cardiomyopathy and at least one of said genetic variants is selected from the group consisting of, or in linkage disequilibrium with, at least one genetic variant selected from the group consisting of: rs28933099, rs3218713, rs2856655, MTTH Mito: 12192 R, MTTL1 Mito: 3303 Y, MYL2 Chr. 12: 109841320 R, MYH7 Chr. 14: 22968327 R, MYH7 Chr. 14: 22968054 Y, and MYBPC3 Chr. 11: 47320705 S. In some embodiments, said predisposition or carrier status is determined for thrombophilia and at least one of said genetic variants is selected from the group consisting of, or in linkage disequilibrium with, at least one genetic variant selected from the group consisting of: rs6025, rs6046, rs5985, rs1801133, rs1800790, rs2232354, rs9574, rs5985, rs1800595, rs1799963, rs2232698, SERPINA10 Chr. 14: 93824396 R, PROC Chr. 2: 127900253 Y, PROC Chr. 2: 127895484 R, PROC Chr. 2: 127902541 Y, PROS1 Chr. 3: 95080840 Y, PROS1 Chr. 3: 95086439 R, F11 Chr. 4: 187429867 Y, F11 Chr. 4: 187438406 Y, FGA Chr. 4: 155730115 R, FGA Chr. 4: 155727040 R, THBD Chr. 20: 22976686 K, rs5907, FGB Chr. 4: 155706587 R, TFPI Chr. 2: 188057188 Y, PLG Chr. 6: 161079615 R, FGG Chr. 4: 155747369 W, SERPINC1 Chr. 1: 172150331 Y, and SERPINC1 Chr. 1: 172139799 S. In other embodiments, said predisposition or carrier status is determined for Exfoliation Glaucoma and at least one of said genetic variants is selected from the group consisting of, or in linkage disequilibrium with, at least one genetic variant selected from the group consisting of: rs1801133, rs1048661, and rs3825942.

In further embodiments, said individual selects said two or more phenotypes. In another embodiment, said set of genetic variants was identified using a high density DNA microarray. In yet another embodiment, said set of genetic variants was identified by sequencing genomic DNA from said individual. In an embodiment, said individual is a patient. In some embodiments, said individual is a suffering from an unknown disease or condition. In other embodiments, said individual is an organ, cell, or tissue transplant candidate. In further embodiments, said individual has died of unknown causes.

Other aspects provided herein is a Law Enforcement set of probes, wherein said set comprises probes, wherein each of said probes is specifically selected to detect a genetic variant correlated with a Law Enforcement phenotype. In an embodiment of the Law Enforcement set of probes said set detects at least two phenotypes listed in the following figure: Law Enforcement Panel (FIG. 18). In another embodiment of the Law Enforcement set of probes said set comprises at least two probes, and each of said at least two probes detects a different genetic variant, and wherein each of said different genetic variants is correlated to the same phenotype.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 illustrates an overview of a method or business method of providing genetic testing, profiles, and/or analysis.

FIG. 2 depicts a diagram of a sample genetic pedigree. A male individual (proband) is identified on the pedigree by the arrow. The individual's maternal grandfather died from unknown cancer at age 55 and an uncle, on his maternal side, died from prostate cancer at age 58. His paternal grandparents both died in their 50's from unknown illnesses, a paternal uncle died of heart disease around the age of 60, and his father died recently of a heart attack at the age of 72. He states that he has lost contact with his maternal aunt and uncle. His mother has glaucoma and arthritis but is otherwise healthy and his sister and her two children are also healthy. No other family history is given. Genetic Pedigree Analysis may be utilized for genetic counseling. The pedigree may enable healthcare professionals, such as genetic counselors, physicians, nurse practitioners, or physician assistants, to follow disease trends and identify possible at-risk individuals. Males are represented by squares, females as circles, and a line connecting a square and a circle from two different lineages represents a marriage.

FIG. 3 illustrates a Punnett Square where both parents are carriers of a monogenic disease. Normal Allele refers to the allele that is not associated with the phenotype (such as a disease). Disease Allele refers to the allele that is associated with the phenotype (such as a disease). Carrier means the individual possesses one phenotype-associated allele but does not have the phenotype. The individual may pass on a phenotype-associated allele to future generations. Diseased means the individual is ‘Affected’ or ‘Likely to be Affected’ by the phenotype. The individual may pass on a phenotype-associated allele to future generations. ‘Carrier status’ may refer to either being a ‘carrier’ or being ‘affected or likely to be affected’ by a phenotype.

FIG. 4 depicts an information chart for an individual with A) limited information about a subject and B-C) with more information about the subject.

FIG. 5 depicts a sample report of genotypic data. “Rs” numbers are used when the genetic variant and it's surrounding sequence has been included in the public United States' National Center for Biotechnology Information's (NCBI) dbSNP database (accessible at www.ncbi.nim.nih.gov/SNP/) and assigned an “rs number”. If that specific genetic variant is not included in this public dbSNP database, then the genetic variant and its flanking sequence is assigned an “eg” number, which serves as an internal identification number. The genotype column denotes the diploid genotype for that variant (e.g. a genotype of “GA” denotes a heterozygous sequence of guanine and adenine at the position identified by the given variant), DEL denotes a deletion, and INS denotes an insertion.

FIG. 6 illustrates sample internal data reports as well as examples by which these reports can be filtered, such as for A) all conditions or traits, B) GVP ≧1.5, C) monogenic, D) replicated or monogenic conditions, or E-G) phenotypes (“CSR” refers to Clinical Significance Rating; “PIR” refers to Phenotype Impact Rating). For FIGS. 6A-D:

Column 1=Genetic Variant=identifies the specific genetic variant detected. “Rs” numbers are used when the genetic variant and it's surrounding sequence has been included in the public National Center for Biotechnology Information's (NCBI) dbSNP database (accessible at www.ncbi.nim.nih.gov/SNP/) and assigned an “rs number”. If that specific genetic variant is not included in this public dbSNP database, then the genetic variant and its flanking sequence is assigned an “eg” number, which serves as an internal identification number.

Column 2=Genotype=identifies the specific genotype detected during genetic testing for each of the genetic variants in column 1.

Column 3=Gene or Locus=identifies the gene where the genetic variant (from column 1) occurs within or bordering. If the genetic variant occurs within an intergenic region, then the loci where the genetic variant exists is identified.

Column 4=Phenotype=identifies the phenotype associated with the genetic variant (column 1) and its genotype (column 2). This association is ascertained from scientific literature.

Column 5=Phenotype-Associated Genotype or Allele=identifies the allele or the genotype associated with the risk value for that phenotype. This information is ascertained from scientific literature.

Column 6=Population Match?=identifies whether or not the individual's population (such as gender, ethnicity, etc.) matches the population from scientific studies in which the genotype-phenotype association was deduced.

Column 7=Monogenic?=identifies whether the genotype-phenotype association is monogenic or not. This information is ascertained from scientific literature.

Column 8=Monogenic Status=identifies the status (affected or carrier) of monogenic phenotypes.

Column 9=Risk=The risk value associated with the allele or genotype for the genotype-phenotype association. This is ascertained from scientific literature.

Column 10=Risk Type=This identifies the type of risk value from column 8, such as whether it is an odds ratio (OR), relative risk (RR), or hazard ratio (Z). This is ascertained from scientific literature.

Column 11=Absolute Value=this is either an absolute or cumulative value for this genetic variant's specific genotype-phenotype association, as reported in the scientific literature. An example of an absolute value is the new lifetime risk for that individual based on that genotype or an absolute amount associated with the phenotype (as opposed to an odds ratio, relative risk, or hazard ratio), such as a specific genetic variant's genotype being associated with an average systolic blood pressure of 140 mmHg±5 mmHg. In the example of blood pressure, if the blood pressure value is in the hypertensive range, then this would contribute to the CGR and PMR for hypertension as described herein.

Column 12=Absolute Value Descriptor=this identifies exactly what the absolute or cumulative value (from column 11) is. For example, it can be “Cumulative Value” if the value listed in column 11 was a cumulative value, or it can be a lifetime risk at a specific age or age range, if the value listed in column 11 is a lifetime risk at a specific age or age range.

Column 13=Replicated=this identifies whether or not the genetic variant's genotype-phenotype association and its risk value or absolute value has been replicated. If it has been replicated (two or more independent studies have found the same statistically significant genotype-phenotype association and the same direction of risk) then it is assigned a “Yes”, if it has not been replicated yet, then it is assigned a “No”, if it was replicated within a single study (such as if two independent populations were found to have the same statistically significant genotype-phenotype association and the same direction of risk) then it is assigned a “Within” and if the genotype-phenotype association is a monogenic phenotype, then it is assigned “Mono”. If the genetic variant's genotype-phenotype association is not found to be statistically significant in subsequent studies after a study has found it to be statistically significant, then it is assigned “Failed”. If there are three or more studies, where one or more contains data that is contradictory to the other studies (such as if two studies find a statistically significant association between a genetic variant's allele or genotype and a phenotype but a third does not) for the same population, then the studies with the highest power (number of people in the study cohort) are considered most relevant.

Column 14=GVP Score=the GVP Score means the ‘Genetic Variant-Phenotype Score’, which is a value for the degree to which that genetic variant has been replicated in the scientific literature. The description for GVP score appears in FIG. 7.

Column 15=GVP Triage=the GVP Triage means the ‘Genetic Variant-Phenotype Triage’, which is a value that discerns its clinical significance. The descriptions for GVP Triage appear in FIG. 8.

Column 16=GVP Rank=the GVP Rank is the order in which that genetic variant should be utilized in case two or more genetic variants within tight linkage disequilibrium are both detected during genetic testing. If these genetic variants are associated with the same signal, they may give the same risk information about the phenotype association and only one should be included in the calculations and algorithm. The genetic variant designated with a GVP Rank of “1” will always be utilized first, over any other rank. For example, if two genetic variants (X and Y) within the same gene or locus were both detected and both provide the same signal information about the phenotype (as ascertained from the scientific literature or HapMap linkage disequilibrium data or both), and genetic variant X is ranked 1 and Y is ranked 2, only genetic variant X will be utilized in the calculations and algorithm. Genetic variant Y may still also be tested for and/or analyzed because it may give other information about another phenotype, it may be part of a haplotype, it may be part of a panel of variants that are tested and/or analyzed, or the data may be obtained as a consequence of obtaining the data for genetic variant X. If only genetic variant Y is detected but genetic variant X is not, then that means genetic variant Y, with a GVP Rank of 2, will then be used in the calculations and algorithm.

FIG. 7 illustrates a sample of a Genetic Variant-Phenotype (GVP) scoring scheme.

FIG. 8 illustrates a sample of a Genetic Variant-Phenotype (GVP) Triage scoring scheme.

FIG. 9 is a CGR Multiplier and PMR (Predictive Medicine Risk) or NRV (No Risk Value) Multiplier chart.

FIG. 10 is an example of a chart for scores by organ system and an overall genetic health score. The Cumulative Action Score (CAS) can be filled in for more than one organ system and determined for an organ system. The organ system score or Indicator of Genetic Health of an Organ System can be indicated by a color. Red would be used for scores less than −10, indicating highly important to discuss with client and may be highly important for client to follow-up with their physician or specialist based on this information, pink can be used for scores between −1 to −10 to indicate moderately important risk, green can be used for scores of 0 to indicate no pertinent deleterious or protective information discovered although organ system was accessed, blue can be used for scores between +1 to +10, to indicate moderately important protection, gold can be used for scores >+10 indicating very beneficial protection, and no color can be used for an Organ System or Medical Specialty if it was not accessed. The overall genetic health score can be determined by adding all the CAS and dividing by the total number of CASs, which may be used as an indicator for genetic wellness and is also represented by a color as is the Indicator of Genetic Health of an Organ System.

FIG. 11 depicts a schematic of a computer system useful in the methods of the present invention. FIG. 11A is a schematic of a non-limiting example of a computer system that can be used for storing, receiving and analyzing data from genetic results or testing. FIG. 11B is a schematic of a non-limiting example of the general steps for obtaining a genetic analysis of a patient sample from a computer system that can be used for receiving and analyzing genetic data.

FIG. 12 depicts reports generated from an individual tested with the Full Genome Analysis Panel, such as A-B) Risk Assessment reports for Alzheimer's Disease (A) and Macular Degeneration, Age-Related (B), C-D) Carrier Assessment reports for Malignant Hyperthermia (C), and Cystic Fibrosis (D), E) Healthcare Professional Summary and F-G) References.

FIG. 13 depicts reflex testing schematics of A) general reflex testing; B) a Women's Health Panel for Obesity and Leanness, C) a Carrier Screening Panel (Rare Diseases, Orphan Diseases, Metabolic Diseases and/or Syndromes), and depicts matrix reflex testing schematics of D) prostate cancer and of E) Epidermolysis Bullosa Simplex (EBS).

FIG. 14 depicts a schematic of the 2 part analysis for Offspring Projection through the Combined Analyses of Different Individuals (OP-CADI).

FIG. 15 depicts a Full Genome Panel Alpha.

FIG. 16 depicts a Military and Armed Forces Panel Alpha.

FIG. 17 depicts a Military and Armed Forces Panel Beta.

FIG. 18 depicts a Law Enforcement/Forensic/Investigative Panel.

FIG. 19 depicts a Custom Panel, where an individual can choose any disease or trait from any of the panels described herein. An individual can choose different denominations, such as a Custom 10 Panel, which tests for 10 phenotypes or a Custom 20 Panel, which tests for 20 phenotypes. Custom panels can range from one phenotype to over 1,000 phenotypes.

FIG. 20 depicts various options for selection of phenotypes from panels, such as Offspring Projection through the Combined Analyses of Different Individuals (OP-CADI) Option, Only Decreased Risk Option, Only Increased Risk Option, or Specific Disease Exclusion Option.

FIG. 21 depicts example indications that, if present, may suggest genetic testing using the specified panel.

FIG. 22 depicts significant genetic variants and their associated disease or trait.

FIG. 23 depicts journal articles or references reporting an association between a specific genetic variant's allele or genotype and a phenotype.

FIG. 24 illustrates multifactorial phenotype risks which have, for example, both a genetic component and an environmental component as compared to monogenic or polygenic phenotype risks.

### DETAILED DESCRIPTION

Genotypes contribute to phenotypes, such as traits, diseases, disorders, conditions, or characteristics. Genotypes comprising genetic variations, such as allelic polymorphisms or single nucleotide polymorphisms (SNPs), can provide a method of correlating a genotype with one or more phenotypes for an individual. For example, clinically relevant polymorphisms can be used to determine clinically relevant phenotypes, including phenotypes such as the risk or predisposition an individual has for a specific disease, disorder, condition, or trait. Phenotypes may also include the pharmacogenomic profile of an individual including medication metabolism, effectiveness, adverse reactions, dosing indications, and choice of medication. Many phenotypes, such as diseases, disorders, traits and conditions are multifactorial and may be interconnected with other phenotypes. Monogenic disorders can also be interconnected with other phenotypes. A comprehensive, dynamic analysis of an individual genome, combined with environmental factors, can be used to understand the individual's risk or predisposition, carrier status, diagnosis, determination and risk or predisposition to future generations of monogenic, polygenic and multifactorial phenotypes, as well as their interconnectedness with other relevant phenotypes.

Provided herein are methods and systems for generating genetic profiles. The term “genetic profiles” includes genetic analyses and/or genotype profiles. The genetic profiles can provide comprehensive, dynamic genetic analysis for an individual. Genetic profiles can use genetic information from an individual to determine the carrier status of a phenotype or a predisposition or risk for a phenotype. Individuals may be human as well as non-human, such as other mammals, including, but not limited to pets, such as dogs, cats, and birds; farm animals such as pigs, cattle or cows, goats, chickens, ducks, turkey, fish, and sheep, as well as other animals, such as apes, bison, camels, horses (for example, racehorses, such as Harness and Thoroughbred), whales and dolphins. In some cases, the disclosure applies to human individuals. In some cases, the disclosure applies to non-human individuals. In some cases, the disclosure applies to mammals or non-human mammals. Genetic profiles may also be generated for plants, including but not limited to cotton plants, olive trees, evergreen coniferous trees, banana trees, apple trees, orange trees, grapefruit trees, cherry trees, almond trees, wheat, corn, hemp, soybeans and rice. Genetic profiles can be generated for fish, including but not limited to salmon, tuna, sea bass, Alaska pollock, cod, eels, tilapia, flashlight fish, anglerfish or sharks. Genetic profiles can also be generated for invertebrates, such as lobsters, shrimp, scallops and insects; fungi; microorganisms, such as bacteria or viruses; and endangered species or extinct species from which genetic material can be obtained.

A phenotype is any observable, detectable or measurable characteristic of an organism, such as a condition, disease, disorder, trait, behavior, biochemical property, metabolic property or physiological property. The genetic information can also be used to determine the pharmacogenomic profile for an individual. The genetic information can also be used to determine the likelihood or predisposition of an individual or a couple in passing on genes and genetic variants that may contribute to specific phenotypes in their offspring or the likelihood of specific phenotypes occurring in potential offspring through the genetic analysis of different individuals as potential parents. The information may also be used in a second analysis or determination of an individual's carrier status of a phenotype or their risk or predisposition to a phenotype. Knowledge of the risks can be useful to health care providers in evaluating health risks, such as by providing recommendations to improve an individual's health or preventive medicine recommendations that may help decrease the incidence, or delay the onset, of specific diseases in that individual's future. Recommendations may include medical recommendations, as well as recommendations that may include, but are not limited to, changing lifestyle habits, such as dietary changes, exercise regimens, levels of stress and stress reduction and the like. Risks or predispositions can be reflected by scores or other numerical values. For example, the score or numerical value may be scaled to express the level of risk or predisposition to a phenotype, such as a medical condition or a non-medical condition.

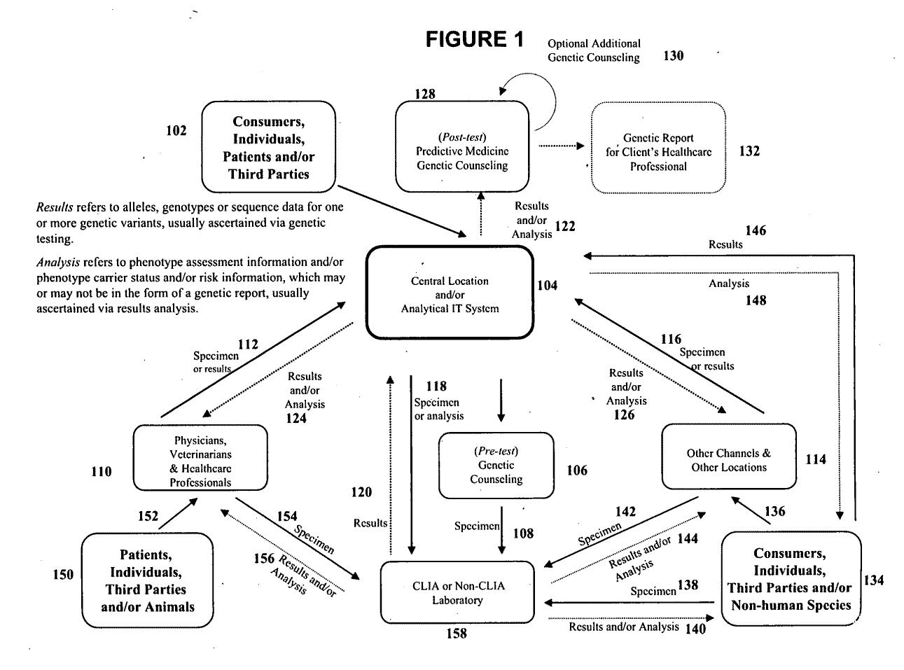

FIG. 1 illustrates some general and non-limiting steps involved in genetic analysis. Samples or specimens, such as any biologic specimen or biologic material, may be taken at the central location (104) and after or before payment, submitted for processing (112 or 116) at a sample processing facility (108) such as a laboratory (158) that may processes the sample, conduct the genetic testing and/or generate the results (such as raw genotypic data or genetic analysis) (120, 156, 144). The laboratory (158) may adhere to appropriate governmental agency guidelines and requirements, for example, in the United States, a processing laboratory may be regulated by one or more federal agencies such as the Food and Drug Administration (FDA) or the Centers for Medicare and Medicaid Services (CMS), and/or one or more state agencies. In the United States, a clinical laboratory may be accredited or approved under the Clinical Laboratory Improvement Amendments of 1988 (CLIA). Samples may also be obtained from individuals at other locations such as health care facilities (110) or directly from the individuals themselves (102, 134). Samples may also be obtained from other channels or facilities (114), e.g., DNA storage bank, blood bank, tissue bank, tissue repository, crime scene, pathology laboratory, morgue, archeological site, or other location. For example, ‘ancient DNA’ may be found at an archeological dig site. Thus, at times, the actual ‘individual’, such as a person or animal or other organism, may not actually be present when the sample is collected. In some embodiments, the nucleic acid may be provided from the individual, or third party, as a sample, which sample may have been previously obtained, i.e. prior to performance of the method of the invention (102).

Other channels or facilities (114) also may include facilities such as spas, medical spas, gyms, fitness centers, weight loss centers, clinics, kiosks, nurses offices, schools, governmental agencies or offices, programs, crime scenes, prisons, jails, military locations, ambulances, hospitals, medical centers, doctor's office, clinics, fertility centers, assisted reproductive technologies centers, sperm banks or donation centers, egg donation centers or programs or companies, prenatal testing companies, business locations, corporate locations, bench research centers, clinical research centers, pharmaceutical companies, places of military, police, or clandestine operations, an individual's house, wellness centers, longevity centers, space centers, executive health programs, funeral homes, veterinarian's offices, veterinary clinics, veterinary hospitals, farms, ranches, natural habitats, archeological digs, archeological centers, museums, cemeteries, or industrial locations. Such facilities may themselves collect samples or specimens (112, 116) from individuals or animals or any organism or from the sample's place of occupancy as stated herein and submit to a central (104) location after or before payment, where the samples are then submitted to a laboratory (158), such as a CLIA laboratory or a non-CLIA laboratory, for processing. Alternatively, the sample may be sent directly from the place of sample collection (104, 110, 114, 134) to a laboratory (158) (either CLIA or non-CLIA certified laboratory) where the genetic testing and/or genetic analysis then occurs or the sample may undergo genetic testing and/or genetic analysis at the sample collection site (104, 110, 114, 134) itself. Optionally, before the testing or analysis of his or her genome, an individual may receive “pre-test” genetic counseling (106). Following such counseling, the specimen may be sent to a CLIA or NON-CLIA laboratory (108). In some cases, an individual may send either his or her genetic testing results directly to the Central Location (146), where such results may be further analyzed, compiled into a report, and sent or transmitted back to the individual (148).

As also illustrated in FIG. 1, a physician, veterinarian, or other healthcare professional (110) may obtain a biological specimen from a patient, individual, third party or animal (150, 152) and may send it to either a central location (112, 104) or to a laboratory (154, 158) for genetic testing and/or analysis in order to ascertain the genotype of one or more genetic variants throughout the genome and, optionally, in order to correlate the genotype with one or more phenotypes. The central location or laboratory may also be a site where methylation status, epigenetic factors at one or more genetic variants throughout the genome, karyotype and/or cytogenetic properties are evaluated. The results of the genetic testing or the genetic analysis (e.g., a genetic analysis contained in a genetic report) (124) may then be sent and/or transmitted to the physician, veterinarian, health care professional and/or individual or patient (110). Alternatively, the genetic testing may have already been completed, either at the time or in the past, and the results of the genetic testing, such as genotypic results may then be sent or transmitted to a central location or analytical IT system (112) where genetic analysis may be performed. The genetic analysis (such as a genetic report) may then be sent or transmitted (124) to the physician, veterinarian, healthcare professional or the patient (110) or to another location (114).

A consumer, individual, or third-party (134) may collect a biological specimen on his or her own as described herein and send the specimen (138) to the laboratory (158). The laboratory may then perform genetic testing on genetic material isolated from the biological specimen (or the biological specimen may already be genetic material, such as isolated DNA) in order to determine one or more genetic variants throughout the genome and will send and/or transmit the results and/or the analysis (such as a genetic report, if the laboratory also conducts the analysis) back to the consumer, individual or third party (140). If the laboratory does not conduct the analysis, then the laboratory (158) may send the genetic testing results (120) to a central location and/or analytical IT system (104) that then may conduct the genetic analysis and may send the analysis either back to the laboratory (118) that may then return the analysis (140) to the consumer, individual, or third party (134) or the central location and/or analytical system may send or transmit the analysis (148) (such as a genetic report) to the consumer, individual, entity, or patient (134). Alternatively, the consumer, individual, third party, and/or non-human species (134) may already have results from genetic testing (such as from current or recent genetic testing or genetic testing done anytime in the past) and may send the results of this genetic testing (146) to a central location and/or Analytical IT System (104) that then may analyze the results and send or transmit or both the analysis (such as a genetic report) (148) to the consumer, individual, or third party (134).

Any results obtained at the Central Location (104), may also be sent to yet another location, where post-test predictive medicine genetic counseling is conducted (128). A genetic report describing genetic analysis or genetic tests and containing other information described herein may then be sent or transmitted to the individual, or to another third party, such as the individual's healthcare professional (132).

A consumer, individual, third party and/or non-human species may either visit, or be taken to, a location that extracts a biological specimen (as described herein) or leave a biological specimen (136) at a location (114), either willingly (such as donating sperm to a sperm bank or donating a tissue sample to a tissue bank) or unwillingly (such as being a victim of a crime that leaves blood or other bodily fluid at the scene of a crime or a biological sample discovered at a place of archeological excavation and/or investigation) and this biological specimen may then be sent (142) to a laboratory (158) or the specimen may be sent (116) from the location (114) to a central location and/or analytical IT system (104) where it may undergo genetic testing (such as with a lab on a chip handheld device) or stored or the specimen may be sent (118) to a laboratory (158) to be stored or for testing. The results of the genetic testing may then be sent or transmitted (120) to a central location and/or analytical IT system (104) or to the consumer, individual, or third party (140, 134) or to the location (114).

The results may be analyzed at the central location and/or analytical IT system (104) and then the analysis (such as a genetic report) is sent and/or transmitted (126), back to the location (114), which may be the same location (such as a forensics laboratory) or a different location (such as a government building or a police station). The location (114) may also already have the results from current or previous genetic testing and may send or transmit the results (116) to a central location and/or analytical system (104) where the results are analyzed and then the analysis is sent or transmitted (126) back to the location (114), which can be the same location that sent the results or a different location (for example, the results may have been sent or transmitted (116) by a police station (114) and the analysis (such as a genetic report) is sent or transmitted the Federal Bureau of Investigation headquarters (114), or the analysis can be sent or transmitted or both to more than one location, such as to the police station (114), the FBI headquarters (114), a prison (114) and/or a hospital or physician's office (110). Genetic testing results or analysis (such as a genetic report) or both may be sent or transmitted or both back to the same location that sent the specimen or to a different location or they may be sent or transmitted to multiple locations at once or at different times. The genetic specimen may also be stored at various locations (104, 110, 114, 158, 134) for a defined amount of time (such as one year) or indefinitely. The results or the analysis or both may also be stored at various locations (104, 110, 114, 158, 134) for a defined amount of time (such as one year) or indefinitely.

Alternatively, the laboratory (158) may refer to a desktop device or machine that exists within the field or an office or home setting, or other location, such as within the office where the biologic sample is taken or received or both (102, 104, 110, 114, 134, 150, 158). The laboratory may also refer to a handheld device that analyzes either the purified DNA sample or the unprocessed biologic specimen or both, as is currently being developed, such as “lab on a chip” technology (see for example, Karlinsey and Landers, Lab Chip, 8: 1285 (2008)). The genetic testing to ascertain specific alleles or genotypes or both of specific genetic variants or for partial exome, full exome, or full genome sequencing may occur on this desktop or hand-held device or the analysis itself of the genetic variants, their genotypes, and their association with phenotypes, or both, may either in part or in whole occur on the device, and the desktop or handheld device may display or print out all the results or a subset of the results of the genetic testing, such as specific phenotypes, such as the diagnosis or carrier status of specific diseases or traits or the risk of specific diseases or traits. Conducting genetic testing utilizing a desktop or handheld device may allow for rapid genotype or associated phenotypes to be analyzed and elucidated or both genotyping (genetic testing) and phenotyping (analysis), results to be reported, analyzed, understood, or conveyed to the healthcare provider or any person operating the device or requesting the testing or analysis or both. This may allow for rapid genetic testing, analysis, and genetic reports to be generated at the patient's bedside, such as in the emergency room, at an accident scene, such as by an emergency medical technician, at a mall, kiosk or other business location, such as by a sales associate, at a security entrance or to confirm identity and to guard access to any location or material at any time, such as by an automated machine or by a security guard or by an immigration or customs official, at a person's home, such as by the person themself or a relative of the person, on a battle field, such as by a soldier or medic or military physician, or at a crime scene, such as by a crime scene investigator, forensic investigator or medical examiner.

In some embodiments, the laboratory (158) processes the sample to isolate the genetic material needed for genetic testing and runs the genetic testing to generate a raw genetic genotype profile (that provides the genotypes or specific alleles at one or more places within the genome). The biological sample can be any sample from the individual in which genetic material may be isolated. Such biological samples include, but are not limited to, blood, hair, skin, saliva, semen, urine, fecal material, sweat, tears, buccal tissue, tongue cells, epithelial cells, and various bodily tissues (e.g., a buccal swab, hair follicle, saliva sample, epithelial cells, genetic material, DNA, or blood). The tissue or DNA sample may be directly collected by the individual (134), for example, a buccal or cheek sample may be obtained by the individual taking a swab against the inside of their cheek. Other samples such as a hair follicle, saliva, semen, urine, fecal material, or sweat, may also be supplied by the individual themselves (134). Other biological samples may be taken by a physician, veterinarian, or health care specialist, such as a phlebotomist, genetic counselor, nurse or physician, physician assistant, nurse practitioner, or other healthcare provider or specialist providing access to the genetic testing and analysis service (110, 104). For example, blood samples may be withdrawn from an individual by a nurse. Biological samples may also be taken by other individuals, such as, for example, a medical examiner, a police officer, a crime scene investigator, an archeologist, a medic, or a government official (114). Tissue biopsies may be performed by a physician, veterinarian, or health care specialist (110), and kits may also be available to health care specialists to efficiently obtain samples. A small cylinder of skin or tissue may be removed or a needle or scalpel or swab or adhesive may be used to remove a small sample of tissue or fluids. Blood or other bodily fluid may be collected from a crime scene by swab or field kit or other collection apparatus by, for example, a detective, officer of the law, forensic investigator, or medical examiner (114).

The sample may be obtained at any time either at one of the locations described herein or at any other location not described herein. While the genetic testing of the sample (to obtain genotypic data) may have also occurred, either at a CLIA or non-CLIA laboratory or at any other location, such as the sample collection site (104, 110, 114, 134), in the past (so that some or all of the genotypic data may be already known) or may occur at the present time, such as at a CLIA or non-CLIA laboratory (158) or other facility or at the sample collection site itself, the genetic analysis of the genotypic data to ascertain phenotypic data may occur either at a separate time or at the same time as the genetic testing. The genetic analysis may occur at the same or different location from where the sample is obtained and the genetic analysis may occur at the same or different location from where the genetic testing occurred or is occurring and the. For example, the sample collection, genetic testing and analysis may all both occur at the health care professional's office (110) or the sample collection may occur at the health care professional's office (110), the genetic testing may occur at a CLIA or non-CLIA laboratory (158), and the genetic analysis may then occur at a central location (104) or at the via interaction with a physician, veterinarian or healthcare professional, such as at a physician's or veterinarian's office (110). As another example, the sample collection, such as blood, may occur at a crime scene (114) years after the blood was actually left at that location and when the individual the blood is from is not currently present, the genetic testing may then occur at the present time at a central location (104), and the genetic analysis may occur immediately following the genetic testing, also at a central location (104) and then the results of either the genetic testing or the genetic analysis or both, such as contained within a genetic report, may then be conveyed to the individual or company or agency or governmental body that ordered the genetic testing (104) or the genetic analysis or both either immediately following the genetic testing and/or analysis or at a later time. Alternatively, the genetic testing or the genetic analysis or both may have occurred at a laboratory (158), such as a CLIA or non-CLIA laboratory.

Just as the specimen collection, genetic testing, and genetic analysis may all occur at the same location or at one or more different locations or all at different locations, the specimen collection, genetic testing, and genetic analysis may also all occur at the same time, at one or more different times, or at all different times. For example, specimen collection may occur at time A, with genetic testing occurring instantaneously or seconds, minutes, hours, days, weeks, months, years, decades, centuries, millennia later at time B and genetic analysis may then occur instantaneously as well or may occur seconds, minutes, hours, days, weeks, months, years, decades, centuries, millennia later at time C. As another example, a biological sample detected in permafrost or a mummy from an archeological site may provide a sample of DNA that may be very old, referred to as ‘ancient DNA’, and this biological sample may then be sent to a laboratory (158) where genetic testing occurs with some initial preliminary analysis. However, the genetic testing results may then be stored for a number of years or decades and either the biological sample may undergo genetic testing again and then analyzed or the original genetic testing genotypic data may be reanalyzed at this later time point. The results of the genetic testing or genetic analysis or both may be stored or conveyed or both to the individual or agency or government who ordered or paid for the test, or both.

Reflex testing, OP-CADI (both of which are terms that are described further herein), and/or testing for specific phenotypes by utilizing specific genetic variants or panels may also apply to one or more of the following: desktop or handheld genetic testing and/or analysis and/or reporting. This type of laboratory (158) and/or handheld device may or may not fall under certain regulations, such as governmental regulations, or have to satisfy certain quality control, or governmental, requirements.

An individual's risk or predisposition for a phenotype may include his or her risk for a monogenic phenotype. In some embodiments, an individual's risk or predisposition for a phenotype includes his or her risk or predisposition for polygenic or multifactorial phenotypes. In such cases, the likelihood of developing a phenotype (e.g., disease, disorder, condition or trait) can be calculated based on an individual's alleles or genotypes for one or more genetic variants associated with polygenic or multifactorial phenotypes, and may also include analysis of non-genetic factors such as environment and/or lifestyle habits (e.g., smoking habits, alcohol use, exercise habits, body mass index, obesity levels, diet, sun exposure or exposure to physical or mental stress). Additional examples of these factors are described herein.

Risk may also be referred to as a predisposition. Risks may also be expressed as a percentage for an indication of the likeliness of the chance event, such as a medically defined phenotype, such as a condition or a non-medical phenotype, such as a trait, to occur. Risks scores can also be provided with a confidence interval, a statistical value such as a p-value, Z-score, correlation (e.g. R or R²), chi-square, f-value, t-value or both a confidence interval and a statistical value, indicating the strength of correlation between the score and the condition or trait thereof. Scores can be generated for an individual's risks or predispositions for medical conditions based on an individual's genetic profile. Scores can be determined for a specific phenotype (e.g., disease, disorder, condition or trait), for an organ system, for a specific organ, for a combination of phenotypes (e.g., a combination of phenotypes listed in one or more of the panels provided in FIG. 15-18), for a combination of phenotype(s) and organ(s) or organ system(s), for overall health, or for overall genetic predisposition to or risk of specific phenotypes. The phenotype may be a medical condition, for example, scores can be generated for an individual's risks or predispositions for medical conditions based on an individual's genetic profile. Alternatively, scores can be for non-medical conditions, or for both medical and non-medical conditions. Scores may be generated by methods known in the arts, such as described in PCT Publication WO2008/067551 and US Publication No. 20080131887 (each of which is incorporated by reference in its entirety) methods such as described herein, or variations and combinations thereof. In some cases, the risks may be determined using a machine such as a general purpose computer or a special purpose computer using instructions provided on computer readable medium. Inclusion of the specific algorithms described herein to analyze the genetic information and calculate scores representing risks, predisposition to a phenotype and/or overall health profiles, for example, transform a general purpose computer into a special purpose computer for analyzing the genetic variants identified. Such algorithms can be provided in any combination to execute those functions desired by a client. Thus, the computer system may include some or all of the computer executable logic encoded on computer readable medium to instruct the computer system to complete the analysis, evaluations, scoring of the identified genetic variants, recommendations and reports for the client as desired.

In some embodiments, the calculated or determined risk or predisposition of one or more specific phenotypes from an individual's genetic profile provides a measure of the relative risk or predisposition of that individual for one or more phenotypes, as further described herein. The relative risk may be determined as compared to the general population or as compared to a control (e.g. a different individual) lacking one or more of the genetic variants identified in the individual's genetic profile. Additional examples and further description of risk and risk scores are provided herein.

In some cases, an individual with an increased relative risk or predisposition for a specific phenotype may be an individual with an odds ratio of greater than 1 for the specific phenotype, for example an individual with an odds ratio of about 1.01, 1.05, 1.1, 1.2, 1.5, 2, 2.5, 3, 4, 5, 6, 7, 8, 9, 10, 15, 20, 25, 50, or 100 or more for developing a phenotype relative to the general population or a control individual. In some cases, an individual with an increased risk or predisposition may be an individual with a greater than 0% increased probability of a phenotype, for example an individual may have a 0.001% greater probability of a phenotype based on their genetic profile, a 0.01% greater probability, a 1% greater probability, a 5% greater probability, a 10% greater probability, a 20% greater probability, a 30% greater probability, a 50% greater probability, a 75% greater probability, a 100% greater probability, a 200%, 300%, 400%, 500% or more greater probability of a phenotype relative to the general population or a control individual. In some cases, an individual with an increased risk or predisposition may be an individual with a greater than 1 fold increased probability of a phenotype relative to a control individual or the general population such as for example about a 1.01 fold, 1.1 fold, 1.2 fold, 1.3 fold, 1.4 fold, 1.5 fold, 2 fold, 3 fold, 5 fold, 10 fold, 100 fold or more increased probability of a phenotype relative to a control individual or the general population. Increased risk or increased predisposition may also be determined using other epidemiological methods such as for example calculation of a hazard ratio or a relative risk.

In some cases, an individual with a decreased risk or decreased predisposition for a specific phenotype is an individual with an odds ratio of less than 1, for example 0.99, 0.9, 0.8, 0.7, 0.5, 0.4, 0.2, 0.1, 0.01 or lower odds ratio relative to a control individual or relative to the general population. An individual with a decreased risk or predisposition for a specific phenotype may be an individual with a lower percentage probability than a control individual or the general population for a phenotype. For example, the individual may have a 0.1% lower risk, 1% lower risk, 5% lower risk, 10% lower risk, 15% lower risk, 25% lower risk, 30% lower risk, 40% lower risk, 50% lower risk, 75% lower risk, or 100% lower risk than a control individual or the general population for a phenotype. An individual's decreased risk or predisposition may also be determined as a hazard ratio or a relative risk.

An individual's genetic profile and scores can be used by third parties such as for example, genetic counselors (GCs) and medical professionals such as, for example, physicians, physician assistant, nurse practitioner and medical specialists, or veterinarians (if the genetic testing is conducted on animals) in providing recommendations based on an individual's genetic profile. The genetic profiles and scores can also be used by fitness instructors, athletic coaches, therapists, chiropractors, acupuncturists, weight loss specialists, nutritionists, and the like in providing recommendations to an individual. Fitness instructors, athletic coaches, chiropractors, acupuncturists, weight loss specialists, nutritionists, therapists, psychologists, behaviorists, and the like, can also consult with physicians and medical specialists in providing recommendations to an individual. The recommendations may aid in reducing the overall risk or predisposition to harmful or unwanted phenotypes, or in increasing the risk or predisposition to beneficial or wanted phenotypes. Recommendations may also be for increasing compatibility in relationships, mate selection for increased success or compatibility in relationships or in childbearing decisions, mate pairing to produce offspring with a greater likelihood of desired phenotypes or a decreased likelihood of undesirable phenotypes or both, and others.

The genetic profile for an individual can have information on one or more specific phenotypes. Examples of other numbers of phenotypes included in a genetic profile are described herein. In some cases, a genetic profile can have a “score” that indicates a general risk or predisposition to the specific phenotype or to a group of phenotypes. The specific phenotype can be monogenic or multigenic (polygenic). The phenotype can also be multifactorial.

The phenotypes/conditions analyzed may include clinical and non-clinical phenotypes. Phenotypes/conditions can include medical conditions such as diseases and disorders, e.g., described herein. Phenotypes can also include specific traits. Specific traits may include physical traits (e.g., hair color, weight, height, athletic ability), physiological traits (e.g., lung capacity, drug metabolism, drug sensitivity, longevity), mental traits (e.g., memory retention, intellectual ability), personality and emotional traits (e.g., ability to control anger, novelty seeking behavior, risk-taking behavior, degree of altruism), ethnicity, ancestry (e.g., an individual's place of origin and individual's ancestor's place of origin), age (e.g., age expectancy, or age of onset, of different phenotypes, such as conditions and traits), and any other phenotype, such as diseases, disorders, or traits.

Some phenotypes concern an age of onset. “Age of Onset” may refer to the age that the phenotype is most likely to manifest or the age at which symptoms will first become noticeable and therefore the disease may be diagnosed. Age of Onset may be an approximate age, such as approximately 65 years old for the age of onset of Alzheimer's Disease, Late Onset, or it may be an age range, such as between 12-15 years old for the age of onset of weight loss associated Bulimia Nervosa, or it may be younger than or older than an age, such as age of onset of breast cancer in women older than the age of 50. In some cases, phenotypes include clinical status phenotypes. For example, methods are provided herein for calculating risk or predisposition to phenotypes related to worsening clinical outcomes. Worsening clinical outcomes include but are not limited to a worsening BODE score and/or a decrease in exercise capacity as a result of lung volume reduction surgery in Emphysema patients, clinical improvement (reduction in BODE score and/or increase in exercise capacity) following lung volume reduction surgery in Emphysema patients, protection against, or increased risk of, cognitive decline after coronary artery bypass graft surgery, and protection against or increased risk of recurrence of Crohn's Disease after Surgery-induced remission.

In some embodiments, the genetic profile includes a score that indicates a risk or predisposition of an individual for one or more multifactorial phenotypes. The multifactorial inheritance of a phenotype is based on the interaction between genes and the environment. The genetic factors may be a number of genes; a number of genetic variants within the same or different genes or elsewhere within the genome that is not within a gene; the non-genetic factors may be environmental exposures (e.g., sun exposure, living or working conditions in a high pollution environment); lifestyle habits (e.g., tobacco smoking, alcohol drinking, diet, exercise regimen); or specific traits (e.g., age, gender, national origin, ethnicity, body mass index). Other factors that may also be included in the risk analysis of multifactorial phenotypes include abnormal or suggestive results from a medical examination or test (e.g., high blood pressure, low blood pressure, abnormal heart rate, suspicious skin lesion, suspicious lesion on radiologic examination, abnormal thyroid function test, abnormal egg or sperm morphology, a positive score on a test or questionnaire indicative of substance abuse, a palpable mass upon physical examination, such as during a breast examination); physical or mental symptoms (e.g., pain, fatigue, fever, rash, nausea or vomiting, diarrhea, constipation, dizziness, headache, myopathy, ataxia, anxiety, depression, difficulty focusing); specific medical condition or medical history (e.g., peridontitis, atherosclerosis, heart disease, cancer, inflammatory bowel disease, diabetes, depression, miscarriage); family history (e.g., family history of neurodegenerative disease, cardiovascular disease, sudden death or other disease or disorder) or other genetic or non-genetic factor, e.g., any factor listed in FIG. 21. For example, an individual may have a genetic variant that predisposes the individual to lung cancer only if the individual smokes cigarettes. The individual does smoke daily and therefore this combination of a genetic predisposition and an environmental factor (the lifestyle habit of smoking cigarettes) increases the individual's predisposition to lung cancer and is factored into a score for the individual's risk of lung cancer.

As described herein and as shown in FIG. 24, phenotypes may be monogenic, polygenic or multifactorial.

FIG. 24 shows that for a multifactorial phenotype, the total risk is composed of genetic and environmental factors. The amount that genetics or the environment contributes to this risk differs by phenotype. For example, one phenotype may be determined by approximately 70% genetics and approximately 30% environment while another phenotype may be determined by approximately 40% genetics and approximately 60% environment. The amount that genetics contributes to a phenotype is called the phenotype's heritability. Heritability for a specific phenotype may be determined from various scientific studies, such as twin studies or parent-offspring regression, and the heritability of specific phenotypes can be found in published scientific literature, such as journal articles.

An individual's risk or predisposition for polygenic or multifactorial phenotypes can be calculated based on the allele or genotypes for one or more genetic variants associated with polygenic or multifactorial phenotype(s).

By determining genetic risk or predisposition for multifactorial phenotypes, one can identify those individuals at higher risk due to their genetics and then proactively adjust their modifiable environmental risk, for example, by modifying lifestyle, modifying medications, conducting screening exams, and instituting other lifestyle or living changes. This approach can empower individuals, physicians, and health-care providers and enable them to identify environmental risk modifications that will be of the most value. Although genetic risk may remain unchanged, decreasing environmental risk may have the effect of decreasing risk overall, thereby decreasing the incidence of that phenotype, delaying its onset, or decreasing its morbidity or mortality.

Some phenotypes have a larger genetic component while others have a larger environment component, but risk for multifactorial phenotypes is always a combination of both of these components. Non-limiting examples of multifactorial diseases include Late-onset Alzheimer's Disease, Prostate Cancer, Breast Cancer, Stroke, Bipolar Disorder, Latex Allergy, Crohn's Disease and Myocardial Infarction.

Similarly, the genetic basis for hundreds of monogenic phenotypes, such as diseases, have been known for years, but widespread screening for individuals carrying or affected by these phenotypes has never before been technologically feasible or cost-effective. By identifying individuals who carry phenotype-related genetic variants, providers may offer extensive family planning options. Previously, there has not been such an ‘early warning system’ for such a large number of monogenic phenotypes.

In some embodiments, individuals are informed of the monogenic diseases that they carry and may pass on to future generations. In some embodiments, individuals who are unknowingly already affected by monogenic diseases and whose initial symptom may be sudden death without preemptive medical intervention may be identified. Non limiting examples of monogenic diseases include Tay-Sachs Disease, Cystic Fibrosis, Huntington's Disease, many forms of mental retardation, Long QT Syndrome, Arrhythmogenic Right Ventricular Dysplasia, and some forms of Parkinson's Disease.

A genetic profile is determined by obtaining the genetic information of an individual and correlating the genetic information to a specific phenotype. A specific phenotype may be correlated to one or more genetic variants and their allele or genotype. Genetic markers and variants may include different numbers of nucleotide repeats, nucleotide insertions, nucleotide deletions, single nucleotide polymorphisms, multiple nucleotide length polymorphisms, chromosomal translocations, chromosomal duplications, length of telomeres, copy number variations, or any combination thereof. Copy number variation may include individual or multiple exons or other parts of a gene, an entire gene, multiple genes, microsatellite repeats, nucleotide repeats, centromeric repeats, or telomeric repeats.

Genetic markers and variants may also include epigenetic factors, such as methylation status. Genetic variants may also be changes to a single nucleotide, referred to as point mutations or polymorphisms or mutations or variants, such as single nucleotide polymorphisms, or SNPs. Genetic variants may also be changes to multiple nucleotides, such as changes to two or more nucleotides that are located next to each other or are not located next to each other. Genetic variants may also be the deletion or insertion of one or more nucleotides anywhere within an individual's genetic code, referred to as a deletion or insertion, or deletion insertion polymorphisms, or DIPs (also referred to as indels). Genetic markers and variants may include changes to nuclear DNA, mitochondrial DNA or combinations thereof. Genetic markers and variants may also occur in genetic sequences that are not contained within a cell, such as from lysed cells at a crime scene or if genetic sequences are detectable in the blood or plasma, such as when fetal oligonucleotides exist within maternal blood. At times, genetic sequences, such as DNA or RNA or cells containing DNA or RNA, from one organism may occur within another organism and be able to be isolated or analyzed, such as when fetal cells can be detected and isolated from maternal blood during pregnancy, or such as with hematophagy when one organism, such as an insect, contains blood from another organism, such as within its stomach, and genetic analysis and a genetic profile can be determined from this source of genetic information as well. For non-human species, the genetic profile may be determined by obtaining genetic information from any source of genetic information, such as DNA or RNA, which may exist anywhere within the organism, such as within the cytoplasm of bacteria, within the nucleus and mitochondria of cells from mammals, within the capsid of viruses or within the nucleus and chloroplast of plants and eukaryotic algae.

Genetic variants may also be in linkage disequilibrium with other genetic variants that are detected or determined for an individual's genomic profile. As described by The International HapMap Project (see for example, www.hapmap.org, The International HapMap Consortium, Nature 426:789-796 (2003), The International HapMap Consortium, Nature 437:1299-1320 (2005); The International HapMap Consortium, Nature 449:851-861 (2007)), nearly every variable site typically results from a single historical mutational event as the mutation rate is very low (of the order of 10⁻⁸ per site per generation) relative to the number of generations since the most recent common ancestor of any two humans (of the order of 10⁴ generations). For this reason, without being bound by theory, each new allele is typically initially associated with the other alleles that happened to be present on the particular chromosomal background on which it arose. The specific set of alleles observed on a single chromosome, or part of a chromosome, is called a haplotype. New haplotypes can be formed by additional mutations or by recombination, such as between maternal and paternal chromosomes, resulting in a mosaic of the two parental haplotypes. The coinheritance of SNP alleles on these haplotypes leads to associations between these alleles in the population, known as linkage disequilibrium, LD. As the likelihood of recombination between two SNPs typically increases with the distance between them, without being bound by theory, on average such associations between SNPs decline with distance. In some cases, strong associations can mean that in many chromosome regions there are only a few haplotypes, which can account for most of the variation among individuals in those regions. In some embodiments, because of strong associations between SNPs in a region, information about common SNPs in a region can be determined through information for a few carefully chosen SNPs in the region. As a result, only a few of these carefully chosen SNPs can be used to identify each of the common haplotypes in a region. Linkage disequilibrium can be applicable to all types of genetic variants, including SNPs, DIPs, nucleotide repeats, translocations, and CNVs, and is also applicable to all species, including humans and non-humans.

The genetic variants described herein may be used to determine specific haplotypes or diplotypes. For example, genetic markers or variants, such as SNPs, nucleotide repeats, insertions, deletions and other as described herein, may be in linkage disequilibrium with genetic markers that have been shown to be associated with specific phenotypes. For example, a nucleotide insertion is correlated with a phenotype and a SNP is in linkage disequilibrium with the nucleotide insertion. Through linkage disequilibrium, a disease predisposing allele cosegregates with a particular allele of a SNP or a combination of particular alleles of SNPs. A particular combination of SNP alleles along a chromosome is termed a haplotype, and the DNA region in which they occur in combination can be referred to as a haplotype block. While a haplotype block can consist of one SNP, typically a haplotype block represents a contiguous series of 2 or more SNPs exhibiting low haplotype diversity across individuals and with generally low recombination frequencies. An identification of a haplotype can be made by identification of one or more SNPs that lie in a haplotype block.

Databases of genetic variants are publicly available from, for example, the International HapMap Project (see www.hapmap.org, The International HapMap Consortium, Nature 426:789-796 (2003), and The International HapMap Consortium, Nature 437:1299-1320 (2005)), the United States National Institutes of Health's National Center of Biotechnology Information's Single Nucleotide Polymorphism database (dbSNP) (see www.ncbi.nlm.nih.gov/SNP/), the United States National Institutes of Health's National Center of Biotechnology Information's Entrez Global Query Cross-Database Search System (see /www.ncbi.nlm.nih.gov/sites/gquery) and the European Bioinformatics Institute and the Wellcome Trust Sanger Institute's Ensembl project (see www.ensembl.org/). These databases provide information on genetic variants and genetic variants in linkage disequilibrium patterns. Thus, linkage disequilibrium data can be ascertained through the data publicly available from the International HapMap Project.

Linkage disequilibrium (LD) can be measured by the variables D and r², such as described by Hill and Robertson (TAG Theoretical and Applied Genetics 38: 226-231 (1968)). The International HapMap provides these measures of LD for genetic variants. For example, r² is a measure of the LD between two genetic variants and the range of r² is from zero to one. Thus, in embodiments using such a system of measure, genetic variants that have greater r² values tend to segregate together, such that two genetic variants that have an r²=1 always appear together.

For some genetic variants that are found to be associated with a phenotype, the specific genetic variant is the cause of that phenotype (that genetic variant is the causal genetic variant). For example, on chromosome 1 in the coagulation factor V gene (F5), there exists a genetic variation (an adenine base appears instead of a guanine, IUPAC nucleotide code R (see Table 1)) that changes amino acid position 506 from an Arginine (Arg) to a Glutamine (Gln) (see Table 2 for IUPAC amino acid codes used herein), which appear in dbSNP as rs6025 (Bertina et al., Nature 369:64-67 (1994)). This genetic variant (called Factor V Leiden) was found to be one of the direct causes of activated protein C resistance, which causes the thrombophilia phenotype. Without being bound by theory, it is thought that any genetic variant that is in tight LD (has a high r² value) with the Factor V Leiden genetic variant may also be associated with thrombophilia.

The sequence for a genetic variant may be from any available database, public or private. For example, the sequence data may be from NCBI Build 36.2 (such as, the human genome reference sequence (ref_assembly)), and the mitochondrial sequence may be from NCBI Genebank #AC_{—}000021.2. For example, a genetic variant for the F5 gene may be referenced as “F5 Chr. 1: 167785673 R”, meaning that the genetic variant exists within or bordering the F5 gene on chromosome 1, at position 167785673 on chromosome 1, and that the base is either an adenine or a guanine. The sequence numbering can be relative to the coordinate systems for each chromosome from NCBI Build 36.2. All coding and abbreviations are based on IUPAC nomenclature. The genomic sequence surrounding this genetic variant on the reverse strand is as follows, with R (A or G) appearing at position 167785673:

TGTAAGAGCAGATCCCTGGACAGGC(R)AGGAATACAGGTATTTTGTCCT
TGA

TABLE 1
IUPAC Nucleotide Codes

| IUPAC Nucleotide Code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | Any base |
| — | gap |

TABLE 2
IUPAC Amino Acid Codes

| IUPAC Amino Acid Code | 3 Letter Code | Amino Acid |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Some associations between genetic variants and risk of disease are based upon a ‘signal’ of risk in the vicinity of that genetic variant. The genetic variant may not be the causal genetic variant (ie. it may not be the exact cause of the phenotype) but because it is in LD with the causal variant, the non-causal genetic variant shows an association with the phenotype. These signals can be used clinically as they can allow for the ascertainment of risk from signals (genetic variants in LD with the causal genetic variant) without the exact causal variant being specifically known at that moment. For example, as described in Zeggini et al. (Nat. Genet. 40: 638-645 (2008)), Zeggini et al. conducted a research study examining genetic variants associated with Diabetes Mellitus, Type II (DMII). They found that both rs2641348 and rs2934381 were associated with DMII, but based on data from the International HapMap Project, they wrote that SNPs rs10923931 and rs2641348 appear to represent the same signal (r²=0.92 in HapMap CEU).

In another example, McCarroll et al. (Nat Genet 40:1107-1112 (2008)) conducted research on the cause of the association (the cause of the signal) that had previously been detected (Parkes et al. Nat Genet 39:830-832 (2007); The Wellcome Trust Case Control Consortium Nature 447:661-678 (2007); Franke et al. Nat Genet 40:713-715 (2008)) between region 5q33.1 (containing the IRGM gene) and Crohn's disease (CD). McCarroll et al. found that a specific genetic variant in LD with previously reported genetic variants (rs13361189 and rs4958847) in the region may be the actual causal genetic variant in that region associated with a predisposition for Crohn disease. They found a common, 20-kb deletion polymorphism upstream of IRGM and in perfect linkage disequilibrium (r²=1.0) with the most strongly CD-associated SNP, that causes IRGM to segregate in the population with two distinct upstream sequences. As a result, their work identified a 20-kb deletion polymorphism as the likely causal variant. Thus, conducting genetic testing either for this deletion directly or for genetic variants rs13361189 or rs4958847 (or any other genetic variants in tight LD with the 20-kb deletion) is likely to give the same information about the same signal. Any one of these genetic variants in tight LD with each other can be used to ascertain a specific predisposition to Crohn's disease in relation to the signal at 5q33.1. As a result, any one of the genetic variants can be tested for, and used to discern whether an individual has a predisposition for Crohn disease based on the specific signal in this region (5q33.1, IRGM gene) of the genome.

Causal genetic variants, or genetic variants in LD with the causal genetic variants, are contemplated herein. For example, genetic variants detected for an individual may be in LD with a causal genetic variant. The genetic variants detected may have an r² value of at least 0.2, 0.4, 0.5, 0.6, 0.7, 0.75, 0.8, 0.85, 0.86, 0.87, 0.88, 0.89, 0.9, 0.91, 0.92. 0.93, 0.94, 0.95, 0.96, 0.97, 0.98, 0.99, or 1 with a causal genetic variant. In some embodiments, the genetic variants detected may have an r² value of at least 0.2, 0.4, 0.5, 0.6, 0.7, 0.75, 0.8, 0.85, 0.86, 0.87, 0.88, 0.89, 0.9, 0.91, 0.92, 0.93, 0.94, 0.95, 0.96, 0.97, 0.98, 0.99, or 1 with published genetic variants that are correlated or associated with a phenotype.

In another aspect of the present invention, methods of using oligonucleotides that specifically detect a genetic variant, either a genetic variant directly correlated with a condition, or a genetic variant in linkage disequilibrium with a genetic variant that is correlated to a phenotype. Preferably, the genetic variant detected by such an oligonucleotide is associated with a phenotype, such as a medical condition. The association of a genetic variant with a phenotype may be from a scientific publication. The genetic variant that is detected can also be correlated to a non-medical phenotype. In another aspect, other genetic variants, such as described herein, may be detected by oligonucleotides specifically selected to detect such genetic variants, wherein the genetic variants are correlated to a phenotype, such as medical conditions, non-medical conditions, or a combination thereof. The genetic variants detected may be, but not limited to, a SNP, an insertion, deletion, copy number variation, or others.

Genetic variants, such as SNPs, that are not available in public databases can also be used to generate an individual's genetic profile. Furthermore, sequences to detect genetic variants may be unique sequences (e.g., those not listed in public databases, such as NCBI's dbSNP Builds 126-129 for example) upstream or downstream (flanking) of a SNP or genetic variant. For example, the sequence may contain sequence information that encompasses about 10, 15, 20, 25, 30, 35, 40, 45, 50, 60, 75, 100, 150, or 200 bps or more immediately upstream or downstream of a SNP or other genetic variant. The genetic profiles can be determined from oligonucleotide sequences wherein at least 5, 10, 25, 50, 65, 70, or 75% of the sequences corresponding to a SNP or other genetic variant are sequences not listed in a public database, for example sequences about 20, 25, 30, 35, 40, 45, 50, 60, 75, 100, 150, or 200 bps or more (upstream or downstream) of the genetic variant. The sequences to detect genetic variants, or the sequence of a genetic variant, such as the deleted sequence of a deletion polymorphism, may be stored in a private database, such as, but not limited to, the Predictive Medicine Database further described below, and illustrated in Example 9. The private database may be constructed to comprise both publicly available SNPs or other genetic variants, such as sequences containing these genetic variants from public databases as well as sequences not available in public databases. The private database may have at least about 100, 1000, 5000, 6,000, 6,500, 7,000, 8,000, 10,000, 15,000, 20,000, 25,000, 30,000, 45,000, 50,000, 100,000, 150,000, 200,000, 250,000, 300,000, 350,000, 400,000, 450,000, 500,000, 750,000, 1,000,000, 1,500,000, 2,000,000, 2,500,000, 3,000,000, 3,500,000, 4,000,000, 4,500,000, 5,000,000, 5,500,000, 6,000,000, 6,500,600, 7,000,000, 7,500,000, 8,000,000, 8,500,000, 9,000,000, 9,500,000, 10,000,000 or more genetic variants, such as SNPs, that are associated with specific phenotypes, such as diseases or traits. The private database may contain SNPs or other genetic variants associated with specific phenotypes, such as diseases or traits, present in at least 100, 250, 500, 750, 1000, 1250, 1500, 2000, 3000, 3500, 4000, 4500, 5000, 5500, 6000, 6500, 7000, 7500, 8000, 8500, 9000, 9500, 10,000, 10,500, 11000, 11500, 12000, 12500, 13,000, 13,500, 14,000, 14,500, 15,000, 15,500, 16,060, 16,500, 17,000, 17,500, 18,000, 18500, 19000, 19500, or 20,000 genes.

The database may contain genetic variants, such as SNPs, present in non-coding regions. The genetic variants, such as SNPs, may be medically related or non-medically related. The genetic variants, such as SNPs, may include only clinically relevant genetic variants, or genetic variants in genes or in linkage disequilibrium with other genetic variants correlated with clinical phenotypes. The SNPs, or other genetic variants, may be organized by medical specialty, organ system, gene, chromosome, location on a chromosome, or phenotype. The SNPs, or other genetic variants, can be organized by clinical severity or by how well that genetic variant is thought to correlate with a specific phenotype or by the degree or status of replication of that genetic variant with its associated phenotype. The private database can also have precise information for each genetic variant, such as a SNP. For example, information such as odds ratio, relative risk, hazard ratio, absolute risk value, applicable populations and ethnicities, inheritance patterns, journal references, journal links, genetic variant synopsis, phenotype information, phenotype prevalence, phenotype incidence, genetic variant allele frequencies, and recommendations or interventions, such as those that have been associated with decreasing the incidence or impact of that phenotype.

In some embodiments, the database is a Predictive Medicine Database (PMD), which can be constructed from, or through a review of some, many, or all published studies throughout some, many, or all worldwide journal articles relating to specific genetic variants associated with a phenotype (disease, condition, trait, process, modifier of other phenotype, and others). The PMD can allow for a an analysis, a comprehensive analysis, or a complete analysis of some, many or all known phenotype-associated genetic variants throughout the partial or entire genome of an individual of any species. The PMD may or may not be part of an Analytical IT System (FIG. 1) (104). An Analytical IT System can process genetic data from genetic testing and/or may analyze genetic information from genetic testing. An Analytical IT System may also process non-genetic data (such as environmental factors) and may include that non-genetic information in the analysis of the genetic data and/or genetic information. An Analytical IT System may associate the genetic information or data with one or more phenotypes. The Analytical IT System may, or may not, include, be part of, or be able to access one or more phenotype matrices, gene matrices, and/or genetic variant matrices (described herein). The Analytical IT System may enable and make possible comprehensive, integrated and/or actionable genetic analysis and/or clinical genetic analysis and/or may enable partial genome analysis, full genome analysis (e.g., whole genome analysis), partial genome clinical analysis and/or full genome clinical analysis (e.g., whole genome clinical analysis).

One or more Analytical IT System(s) may be capable of analyzing genetic data and/or information, such as allele or genotype data for one or more genetic variants within a genome and may be capable of generating an analysis, such as a genetic report (described herein). In some embodiments, a number of PMDs are generated, wherein each PMD is specific for a particular species. For example, a PMD may be provided for humans, and another PMD for canines. The PMD can also be agnostic, in that the data in the PDM can be utilized on any genetic testing platform (such as those provided by Illumina, Sequenom, Agilent, 454 Life Sciences, Pacific Biosciences, Complete Genomics, Helicos BioSciences, Intelligent Bio-Systems, Genome Corp., Genome Diagnostics, Agencourt Bioscience, Microchip Biotechnologies, or Affymetrix) and with any genetic testing methodology (such as arrays, massarrays, beadarrays, microarrays, genechips, PCR, partial or full exome sequencing, and partial or full genome sequencing, such as with pyrosequencing, nanopore, fluorophores, nanopore sequencing, nanoballs, sequencing by synthesis, single molecule real time technology (SMRT)™, true single molecule sequencing technology (tSMS)™, or sequencing by ligation, microfluidics, infrared fluorescence, or other sequencing method or apparatus including others described herein)) and with any genetic testing methodology (such as arrays, massarrays, beadarrays, microarrays, genechips, PCR, partial or full exome sequencing, and partial or full genome sequencing, such as with pyrosequencing, nanopore, fluorophores, nanopore sequencing, nanoballs, sequencing by synthesis, sequencing by ligation, or other sequencing method or apparatus including others described herein). Alternatively, the PMD can also be used only for one or more specific platforms. In some embodiments, all specific genetic variants associated with any discernible phenotype are included within the PMD, including single nucleotide polymorphisms (SNPs), deletion and insertion polymorphisms (DIPs), mutations, repeats, inversions, duplications, copy number variations (CNV), rearrangements, telomere size, and epigenetic factors such as methylation status. The genetic variants may be throughout the entire genome, including those that may exist within or near binding sites, such as transcription binding sites, translation binding sites, or microRNA (miRNA) binding sites, as well as genetic variants that may exist in DNA or RNA within the nucleus, mitochondria, freely within blood or plasma or in the cytoplasm. Genetic variants may also be detected in genetic material that exists in any location in different species, such as contained within the capsid of a virus or within the nucleus or chloroplast of a plant.

The database may be constructed to contain variety of fields dependent upon the particular desired use, the genetic variants being analyzed or the types of scores being provided in the report to the client. Fields of the database are first created and all ascertainable data from each and every journal article is then entered into each of the fields. Nomenclature used in the database can follow the recommendations of The Ad Hoc Committee on Mutation Nomenclature (Human Mutation 8(3): 197-202); Beutler et al. (V. A. M. A. G. M. C. R. S. F. H. Human Mutation 8(3): 203-206 (1996)); Stylianos and Antonarakis (Human Mutation 11(1): 1-3 (1998)); and den Dunnen, S. E. A. (Human Mutation 15(1): 7-12 (2000)). Examples of references, and the phenotypes and genetic loci cited in certain references, are provided in FIG. 23.

Journal articles can be divided by diseases and genetic variants that are monogenic or deterministic (Mendelian variants that directly cause a phenotype, such as genetic variants in the HEXA gene that cause Tay-Sachs Disease) versus those that are polygenic or multifactorial and risk-associated (either increase or decrease risk of phenotype, such as genetic variants in the MC1R gene that increase the risk of skin cancer).

The PMD fields may include: Full Gene Name or Locus (if the genetic variant is not located within or bordering a gene), Gene Symbol, Gene Locus, and Exact Genetic Variant Identification. The Exact Genetic Variant Identification can be the National Center for Biotechnology Information dbSNP rs identifier number (rs#) (see for example, http://www.ncbi.nlm.nih.gov/SNP/), along with the current NCBI Map to Genome Build number and the current NCBI build number for each rs# (such as http://www.ncbi.nlm.nih.gov/mapview/map_search.cgi?taxid=9606). Information about the gene name, symbols, and location and other pertinent information can be found from various NCBI databases, Entrez Pubmed (see for example, http://www.ncbi.nlm.nih.gov/sites/entrez), the Online Mendelian Inheritance in Man® (OMIM®) database (see for example, http://www.ncbi.nlm.nih.gov/sites/entrez?db=omim), the Online Inheritance in Animals (OMIA) database (see for example, http://www.ncbi.nlm.nih.gov/sites/entrez?db=omia) and also the European Bioinformatics Institute and the Wellcome Trust Sanger Institute's Ensembl project (see www.ensembl.org/). Journal articles can be from any journal from around the world that contains published studies of genetic variant-phenotype associations, and may be found through such resources as print version of the journal, libraries, and various internet resources such as through Entrez Pubmed (see for example, http://www.ncbi.nlm.nih.gov/sites/entrez).

Alternatively, the Exact Genetic Variant Identification can be the exact genomic sequence surrounding the genetic variant. For example, it can be the 25, 50, 100, or 200 bp of sequence upstream (5′ flank) of the variant or 25, 50, 100, or 200 of sequence downstream (3′flank) of the variant or both. In some cases, the Exact Genetic Variant Identification can be about 4, 5, 8, 10, 15, 20, 25, 30, 35, 40, 45, or 50 bp of sequence upstream and downstream of the variant. Sources of sequence information can be any available in the arts, such as, but not limited to the Human Genome Project's Reference Sequence, Celera's Sequence, the European Molecular Biology Laboratory-European Bioinformatics Institute-Sanger Institute's Ensembl database (such as from http://www.ensembl.org/Homo_{—}sapiens/index.html) and the National Center for Biotechnology Information database (http://www.ncbi.nlm.nih.gov/gene). The genomic sequence surrounding the genetic variant can be identified according to International Union of Pure and Applied Chemistry (IUPAC) nucleotide ambiguity codes, as described by Cornish-Bowden (“IUPAC-IUB SYMBOLS FOR NUCLEOTIDE NOMENCLATURE” Nucl. Acids Res. 13: 3021-3030.) The genetic variant position on the chromosome relative to the coordinate system, as appears in the European Molecular Biology Laboratory-European Bioinformatics Institute-Sanger Institute's Ensembl database or Entrez Gene database of the National Center for Biotechnology Information's website can also be used, as well as identification of the strand direction of the sequences identified above. An unique internal identification number can also be assigned to each sequence, such as an “eg” number (the letters ‘eg’ followed by a unique number that can be between 1-20 digits long), to facilitate its identification.

Other PMD fields may include location of the genetic variant in or near the gene, such as Intergenic, Intron, Exon, Promoter, Regulatory, Enhancer, 3′untranslated region, 5′untranslated region, Intron Splice Site, Exon Splice Site, or miRNA Binding Site. For genetic variants that exist within or near genes, other PMD fields can include position within gene relative to start codon, amino acid number that the genetic variant occurs within, amino acid change that occurs due to genetic variant according to IUPAC nomenclature (Nomenclature, I.-I. C. o. B. (1966). J. Biol. Chem. 241(11): 2491-249.), and the function of change that occurs, for example, Nonsense, Missense, Sense, Synonymous, Nonsynonymous, Conservative, Non-conservative, Splicing Regulation (Domain Preserved or Abolished).

Other PMD fields may be Allele 1 (specific nucleotide if it is a SNP or nucleotide sequence if it is a DIP or repeat, or copy number if it is a CNV), Allele 2 (specific nucleotide if it is a SNP or nucleotide sequence if it is a DIP or repeat, or copy number if it is a CNV), Phenotype-associated Allele (Specific nucleotide if it is a SNP or nucleotide sequence if it is a DIP or repeat, or copy number if it is a CNV), or Phenotype-associated haplotype or diplotype for two or more genetic variants (if applicable), and Phenotype-associated Genotype (Specific genotype if it is a SNP or nucleotide sequence if it is a DIP or repeat, or copy number if it is a CNV). The haplotype for two or more genetic variants may have all genetic variants and their allele or genotype within the haplotype clearly annotated along with the Phenotype-associated haplotype or diplotype.

Genetic effect and risk prediction algorithm assessment (see for example, Tabor et al. (2002). Nat Rev Genet 3(5): 391-397) can also be a PMD field. Under this field, genetic effect and risk prediction algorithms utilizing one or more from the following may be listed:

A) PupaSuite (Conde et al. (2006). Nucl. Acids Res. 34(suppl_{—}2): W621-625; Reumers et al. (2008). Nucl. Acids Res. 36(suppl_{—}1): D825-829; Yang and Nielsen (2002). Mol Biol Evol 19(6): 908-917), such as PMut (Ferrer-Costa et al. (2005). Bioinformatics 21(14): 3176-3178), Phylogenetic Analysis by Maximum Likelihood (PAML) (Yang. (2007). Mol Biol Evol 24(8): 1586-1591), and/or SNPeffect (Reumers et al. (2006). Bioinformatics 22(17): 2183-2185; Dantzer et al. (2005). Nucl. Acids Res. 33(suppl_{—}2): W311-314);

B) MutDB (Dantzer et al. (2005). Nucl. Acids Res. 33(suppl_{—}2): W311-314), such as Sorting Intolerant From Tolerant (SIFT) (Ng and Henikoff (2003). Nucl. Acids Res. 31(13): 3812-3814) and/or Swiss-Prot (Bairoch and Boeckmann B (1991). Nucleic Acids Res 19:2247);

C) FastSNP (Yuan et al. (2006). Nucl. Acids Res. 34(suppl_{—}2): W635-641), such as Polymorphism Phenotyping (PolyPhen) (Sunyaev et al. (2001). Hum. Mol. Genet. 10(6): 591-597; Sunyaev et al. (2000). Trends in Genetics 16(5): 198-200; Ramensky et al. (2002). Nucl. Acids Res. 30(17): 3894-3900), Transcriptional Factor Search (TFSearch) (Heinemeyer et al. (1998). Nucl. Acids Res. 26(1): 362-367; Akiyama: “TFSEARCH: Searching Transcription Factor Binding Sites”, http://www.rwcp.or.jp/papia/), Exonic Splicing Enhancers Finder (ESEfinder) (Cartegni et al. (2003). Nucl. Acids Res. 31(13): 3568-3571; Smith et al. (2006). Hum. Mol. Genet. 15(16): 2490-2508), RESCUE-ESE (Fairbrother et al. (2002). Science 297(5583): 1007-1013; Yeo et al. (2004). Proc. Natl. Acad Sci. USA 101(44): 15700-15705), FAS-ESE (Wang et al. (2004). Cell 119(6): 831-845), and/or Swiss-Prot;

D) SNPs3D (Yue et al. (2006). BMC Bioinformatics 7(1): 166; Yue and Moult (2006). Journal of Molecular Biology 356(5): 1263-1274; Zhen Wang. (2001). Human Mutation 17(4): 263-270); such as the Stability Model & Profile Model (Yue et al. (2005). Journal of Molecular Biology 353(2): 459-473; Yue and Moult (2006). Journal of Molecular Biology 356(5): 1263-1274);

E) VisualSNP (http://genepipe.ibms.sinica.edu.tw/visualsnp/input.do); and/or

F) FANS (http://fans.ngc.sinica.edu.tw/fans/input.do), which is typically used for unique sequences, i.e. those without dbSNP rs numbers. (C. K. Liu, Y. H. Chen, C. Y. Tang, S. C. Chang, Y. J. Lin, M. F. Tsai, Y. T. Chen and Adam Yao (2008) Functional analysis of novel SNPs and mutations in human and mouse genomes, BMC Bioinformatics, 9(Suppl 12)).

For genetic variants that predispose to a phenotype, such as for multifactorial phenotypes, other PMD fields may include one or more of the following: Risk Value, Risk Type (Odds Ratio, Relative Risk, or Hazard Ratio), Confidence Interval for risk value, p-value of risk value or cumulative or absolute value, Cumulative or Absolute Value (such as an Absolute Value, Absolute Risk or Lifetime Risk); Cumulative or Absolute Value Descriptor; Minor Allele Frequency (MAF) or Haplotype Frequency; Specific Population(s) that the risk and risk-allele (or risk-genotype or risk-haplotype) applies to, incidence of non-phenotype associated allele or genotype in disease cohort, incidence of phenotype associated allele or genotype in control cohort; total number of that specific population within the disease cohort(s); total number of that specific population within the control cohort(s); inheritance (such as Autosomal Recessive, Autosomal Dominant, Multiplicative, Additive, X-linked Recessive, X-linked Dominant, and others); Study Type (such as: Prospective, Retrospective, Genome-wide Association Study, Case-Controlled, and others); and various rating system (as described below) information, such as Replication Status of the genetic variant-phenotype association; Genetic Variant-Phenotype Score Rating (GVP Score); Genetic Variant-Phenotype Triage (GVP Triage) also referred to as the Genetic Variant-Phenotype's Clinical Significance Rating (CSR), and/or SNP Rank.

For genetic variants that are deterministic of a phenotype, such as for monogenic phenotypes, PMD fields may include one or more of the following: Inheritance (such as Autosomal Recessive, Autosomal Dominant, Codominance, Incomplete Dominance, X-linked Recessive, X-linked Dominant, etc.), Replication Status, Genetic Variant-Phenotype Score Rating (GVP Score), Genetic Variant-Phenotype Triage (GVP Triage) also referred to as the Genetic Variant-Phenotype's Clinical Significance Rating (CSR), and/or Study Type (such as: Prospective, Retrospective, Genome-wide Association Study, etc.).

Other PMD fields may include, but not be limited to, Journal Article Author's Name(s), Journal Article's Date of Publication, Name of Journal, Primary Journal Article Reference, World Wide Web (www) address of the pubmed listing of the journal article, World Wide Web (www) address of the actual journal article, and/or References of any other published study on that specific genetic variant-phenotype association. Haplotypes may also be included in the PMD, and each haplotype-phenotype-risk value association may receive its own unique haplotype identifier number. All genetic variants that compose the haplotype may be listed in the PMD, as shown in the fields below. The specific haplotype under its unique identified number can list the genetic variants that compose the haplotype along with the genetic variant's alleles or genotypes that compose the haplotype and are associated with the risk-value for that specific phenotype in that specific population. Selected PMD fields are shown in Table 3.

TABLE 3
Database Categories or Fields
Fields

Type of Study
Exact Journal Article Reference
World Wide Web (www) address for actual article or pubmed listing of the article
Journal Article's Author's Name(s)
Journal Article's Date of Publication
Name of Journal
Institute, Medical Center, or Collaboration that Conducted the Study
What Country or Countries was the Study Conducted Within
References to other Relevant Journal Articles of the Genetic Variant-Phenotype Association
Replication Status
Synopsis & Summation of Journal Article Relevant Results & Information
Gene Name
Gene Symbol(s)
Genetic Variant (dbSNP rs# or internal identifier, such as eg#)
Genetic Sequence (such as 50 bp immediately upstream & downstream of genetic variant if no rs# available)
Chromosome & Locus
Exact Location on Chromosome (such as Ensembl's Coordinate System)
Amino Acid (AA) Change
Location in Gene (such as AA number) or distance from transcription start site
Strand Direction
Allele 1
Allele 2
Allele 3 (if applicable)
Allele 4 (if applicable)
MAF
Prediction of Effect of Genetic Variant Algorithm Value(s)
GVP Rank
GVP Score
GVP Triage
Phenotype
Phenotype-associated Allele(s)
Phenotype-associated Genotype(s)
Inheritance Pattern (Such as Autosomal Rec., Autosomal Dom., X-linked Rec., Multiplicative, etc.)
Risk Value (For phenotype-associated allele or phenotype-associated genotype)
Risk Type (OR, RR, or Z)
Confidence Interval for Rick Value
p-value for Rick Value
Cumulative Value/Absolute Value/Other Value
Cumulative or Absolute Value Descriptor
Geneotype or Allele Associated with Phenotype & Risk Value
Incidence of phenotype associated allele in non-phenotype cohort
Incidence of non-phenotype associated allele in phenotype cohort
Specific Population(s)
Total Aggregate Disease Cohort Study Size(s)
Total Aggregate Control Cohort Study Size(s)
Is this Genetic Variant Part of a Haplotype? (If Yes, reference its Unique Haplotype Identifier Number)
If Part of Haplotype, List Exactly all of Other Genetic Variants in Haplotype
If Part of Haplotype, List Risk-associated Haplotype (alleles) or Diplotype (genotypes)
If Part of Haplotype, Haplotype Risk Value
If Part of Haplotype, Risk Type (OR, RR, or Z)
If Part of Haplotype, Confidence Interval for Rick Value
If Part of Haplotype, p-value for Rick Value
If Part of Haplotype, Cumulative Value/Absolute Value/Other Value
If Part of Haplotype, Specific Population(s)
If Part of Haplotype, Total Aggregate Disease Cohort Study Size(s)
If part of Haplotype, Haplotype Frequency in Population
If Part of Haplotype, Incidence of phenotype-associated haplotype or diplotype in non-phenotype cohort
If Part of Haplotype, Incidence of non-phenotype associated haplotype or diplotype in phenotype cohort

The information for PMD fields may be publicly available, such as through published journal articles, published studies, websites, or from databases such as the aforementioned Entrez Gene database or other Entrez databases, the Ensembl database, the National Center for Biotechnology Information dbSNP database, or the International HapMap Project.

The risks can represent an estimate for an individual to be at risk for, to have, to be a carrier of, or be predisposed to have, a phenotype (e.g., condition, disorder, disease, trait, and the like). The risks or predispositions may be indicated by a numerical value, such as a risk value. The risk value can be an odds ratio (OR), relative risk (RR), hazard ratio (Z), cumulative risk (CR), absolute risk (AR), or lifetime risk (LR). The risk value, or degree of risk, can be expressed in numbers, words, colors, graphs, charts, pictures, or other means, for example, the risk value can be described as high, medium, low, or none. The risk value, or degree of risk, can also be expressed as a range, such as a range of numbers, for example, from −5 to +5, wherein −5 indicates a highly unlikely occurrence of a condition in an individual to +5, wherein there is a highly likely occurrence of a condition in an individual. The risk value, or degree of risk, can also be expressed in a range of colors, for example, red indicating a high risk of having a condition, yellow for no risk, and blue for a decreased risk (protection against) having a phenotype, such as a condition. The number or color ranges can also include numbers or ranges that indicate an individual's genetic profile shows a protective effect for the phenotype, such as a condition. The risk value, or degree of risk, can also be an absolute value (e.g., a systolic blood pressure of 145 mmHg or an age of onset of multiple sclerosis of 45 years old+/−5 years). Further methods of calculating the risk of, carrier status of, or predisposition of an individual for a phenotype are provided herein. Such risks, predispositions and carrier statuses are also further described herein.

The score for a disease or condition can be determined by one or more genetic variants, such as polymorphisms, as well as other factors, such as non-genetic factors, including environmental factors such as living conditions, dietary habits, weight or BMI, age, exercise regimen, lifestyle, medications, or previously known diseases, conditions or traits. One or more scores can be generated for a genetic profile of an individual. An individual's genetic profile can include values or scores for one or more phenotypes, such as diseases or traits. A genetic profile can also include information for selected phenotypes, such as traits or conditions, such as only clinical conditions. Alternatively, a genetic profile can contain information for non-clinical phenotypes only, or a combination of clinical and non-clinical phenotypes. In some cases, an individual has a clinical genetic profile that includes at least 2, 3, 5, 10, 20, 50, 100, 150, 200, 500, or 1000 clinically-relevant phenotypes, such as conditions, diseases or disorders. In some cases, an individual has a clinical genetic profile that includes other numbers of phenotypes, as described herein. A non-limiting example of representative genes and loci included in the present invention is shown in Table 4. Other non-limiting examples of representative genes and loci may include those listed in FIG. 15-18.

TABLE 4
Representative Genes and Loci

| Primary | Alternative | | |
|---|---|---|---|
| Gene/Locus | Gene | | Gene Locus |
| Abbreviation | Abbreviation(s) | Full Gene Name | (NCBI) |
| OCA2 | P | OCULOCUTANEOUS ALBINISM, TYPE II | 15q11.2-q12 |
| | P Gene | | |
| | PED | | |
| | D15S12 | | |
| | BOCA | | |
| CHRNA4 | ENFL1 | CHOLINERGIC RECEPTOR, NEURONAL | 20q13.2-q13.3 |
| | | NICOTINIC, ALPHA POLYPEPTIDE 4 | |
| RYR1 | MHS, | RYANODINE RECEPTOR 1 | 19q13.1 |
| | CCO, | | |
| | RYDR, | | |
| | SKRR | | |
| GABRA2 | | GAMMA-AMINOBUTYRIC ACID RECEPTOR, | 4p13-p12 |
| | | ALPHA-2 | |
| FTO | KIAA1752 | FAT MASS- AND OBESITY-ASSOCIATED GENE | 16q12.2 |
| | FATSO | | |
| GABBR2 | GPR51, | GAMMA-AMINOBUTYRIC ACID B RECEPTOR 2 | 9q22.1 |
| | GABABR2 | | |
| ESR1 | ESR, | ESTROGEN RECEPTOR 1 | 6q25.1 |
| | ESRA | | |
| DMD | BMD | DYSTROPHIN | Xp21.2 |
| | CMD3B | | |
| MYH7 | MYHCB, | MYOSIN, HEAVY CHAIN 7, CARDIAC MUSCLE, | 14q12 |
| | CMD1S, | BETA | |
| | CMH1, | | |
| | MPD1 | | |
| SCN5A | NAV1.5, | SODIUM CHANNEL, VOLTAGE-GATED, TYPE V, | 3p21 |
| | LQT3, | ALPHA SUBUNIT | |
| | IVF, | | |
| | HB1, | | |
| | SSS1, | | |
| | CMD1E, | | |
| | CDCD2 | | |
| MYBPC3 | MYBPC | MYOSIN-BINDING PROTEIN C, CARDIAC | 11p11.2 |
| | CMH4 | | |
| ABCA1 | ABC1, | ATP-BINDING CASSETTE, SUBFAMILY A, | 9q22-q31 |
| | HDLDT1, | MEMBER 1 | |
| | TGD, | | |
| | CERP | | |
| KCNQ1 | KVLQT1, | POTASSIUM CHANNEL, VOLTAGE-GATED, KQT- | 11p15.5 |
| | KCNA9, | LIKE SUBFAMILY, MEMBER 1 | |
| | KCNA8, | | |
| | LQT1, | | |
| | ATFB1, | | |
| | SQT2 | | |
| JAG1 | AGS, | JAGGED 1 | 20p12 |
| | AHD | | |
| APOE | AD2 | APOLIPOPROTEIN E | 19q13.2 |
| KCNE1 | JLNS, | POTASSIUM CHANNEL, VOLTAGE-GATED, ISK- | 21q22.1-q22.2 |
| | LQT5 | RELATED SUBFAMILY, MEMBER 1 | |
| LDLR | FHC, | LOW DENSITY LIPOPROTEIN RECEPTOR | 19p13.2 |
| | FH | | |
| MTHFR | | 5,10-METHYLENETETRAHYDROFOLATE | 1p36.3 |
| | | REDUCTASE | |
| KCNH2 | LQT2, | POTASSIUM CHANNEL, VOLTAGE-GATED, | 7q35-q36 |
| | HERG, | SUBFAMILY H, MEMBER 2 | |
| | SQT1, | | |
| | ERG1, | | |
| | HERG | | |
| F7 | | COAGULATION FACTOR VII | 13q34 |
| RYR2 | VTSIP | RYANODINE RECEPTOR 2 | 1q42.1-q43 |
| | ARVD2 | | |
| | ARVC2 | | |
| DSG2 | HDGC | DESMOGLEIN 2 | 18q12.1-q12.2 |
| | ARVD10 | | |
| | ARVC10 | | |
| PKP2 | ARVD9 | PLAKOPHILIN 2 | 12p11 |
| KRT5 | DDDK5 | Keratin-5 | 12q13 |
| KRT14 | K14 | Keratin-14 | 17q12-q21 |
| COL7A1 | | COLLAGEN, TYPE VII, ALPHA-1 | 3p21.3 |
| MC1R | MSHR | MELANOCORTIN 1 RECEPTOR | 16q24.3 |
| MC4R | | MELANOCORTIN 4 RECEPTOR | 18q22 |
| PKD1 | | POLYCYSTIC KIDNEY DISEASE 1 | 16p13.3-p13.12 |
| CYP17A1 | CYP17, | CYTOCHROME P450, FAMILY 17, SUBFAMILY A, | 10q24.3 |
| | P450C17, | POLYPEPTIDE 1 | |
| | S17AH | | |
| AR | DHTR, | ANDROGEN RECEPTOR | Xq11-q12 |
| | TFM, | | |
| | SBMA, | | |
| | KD, | | |
| | SMAX1 | | |
| POMC | POC | PROOPIOMELANOCORTIN | 2p23.3 |
| GH1 | GHN | GROWTH HORMONE | 17q22-q24 |
| | GHD | | |
| CYP21A2 | CYP21, | CYTOCHROME P450, SUBFAMILY XXIA, | 6p21.3 |
| | CA21H | POLYPEPTIDE 2 (STEROID 21-HYDROXYLASE) | |
| CARD15 | NOD2 | CASPASE RECRUITMENT DOMAIN-CONTAINING | 16q12 |
| | IBD1 | PROTEIN 15 | |
| | CD | | |
| | ACUG | | |
| | PSORAS1 | | |
| IL23R | | INTERLEUKIN 23 RECEPTOR | 1p32.1-p31.2 |
| MUTYH | MYH | MutY, E. COLI, HOMOLOG OF | 1p34.3-p32.1 |
| FSHR | ODG1 | FOLLICLE-STIMULATING HORMONE RECEPTOR | 2p21-p16 |
| F8 | F8C, | COAGULATION FACTOR VIII, PROCOAGULANT | Xq28 |
| | HEMA | COMPONENT | |
| F5 | PCCF | COAGULATION FACTOR V (PROACCELERIN, | 1q23 |
| | | LABILE FACTOR) | |
| SERPINC1 | AT3, | ANTITHROMBIN III | 1q23-q25 |
| | AT-III | | |
| PROC | | PROTEIN C DEFICIENCY, CONGENITAL | 2q13-q14 |
| | | THROMBOTIC DISEASE DUE TO | |
| PROS1 | PSA, | PROTEIN S, ALPHA | 3p11.1-q11.2 |
| | PROSP, | | |
| | PROS2, | | |
| | PSB | | |
| G6PD | G6PD1 | GLUCOSE-6-PHOSPHATE DEHYDROGENASE | Xq28 |
| F13A1 | F13A | FACTOR XIII, A1 SUBUNIT | 6p25-p24 |
| CCR5 | CCCKR5 | CHEMOKINE, CC MOTIF, RECEPTOR 5 | 3p21 |
| | CMKBR5 | | |
| | CKR5 | | |
| SLC26A4 | PDS | SOLUTE CARRIER FAMILY 26, MEMBER 4 | 7q31 |
| | DFNB4 | | |
| GJB2 | CX26 | GAP JUNCTION PROTEIN, BETA-2 | 13q11-q12 |
| | DFNB1 | | |
| | PPK | | |
| | DFNA3 | | |
| | KID | | |
| | HID | | |
| GBA | GBAP | GLUCOSIDASE, BETA, ACID | 1q21 |
| HEXA | TSD | HEXOSAMINIDASE A | 15q23-q24 |
| PAH | PKU1 | PHENYLALANINE HYDROXYLASE | 12q24.1 |
| | HAP | | |
| | PKU | | |
| COL1A2 | | COLLAGEN, TYPE I, ALPHA-2 | 7q22.1 |
| COL1A1 | | COLLAGEN, TYPE I, ALPHA-1 | 17q21.31-q22 |
| CYP19A1 | CYP19, | CYTOCHROME P450, FAMILY 19, SUBFAMILY A, | 15q21.1 |
| | ARO | POLYPEPTIDE 1 | |
| GAB2 | KIAA0571 | GRB2-ASSOCIATED BINDING PROTEIN 2 | 11q13.4-q13.5 |
| BRCA1 | PSCP | BREAST CANCER 1 GENE | 17q21 |
| BRCA2 | FANCD1 | BREAST CANCER 2 GENE | 13q12.3 |
| BARD1 | | BRCA1-ASSOCIATED RING DOMAIN 1 | 2q34-q35 |
| BRIP1 | BACH1, | BRCA1-INTERACTING PROTEIN 1 | 17q22 |
| | FANCJ | | |
| CDH1 | E-CADHERIN, | CADHERIN 1 | 16q22.1 |
| | CDHE, | | |
| | ECAD, | | |
| | LCAM, | | |
| | UVOMORULIN, | | |
| | UVO | | |
| ATM | ATA, | ATAXIA-TELANGIECTASIA MUTATED GENE | 11q22.3 |
| | AT1 | | |
| TP53 | P53, | TUMOR PROTEIN p53 | 17p13.1 |
| | TRP53, | | |
| | LFS1 | | |
| MLH1 | COCA2, | MutL, E. COLI, HOMOLOG OF, 1 | 3p22.3 |
| | HNPCC2 | | |
| MSH6 | GTBP, | MutS, E. COLI, HOMOLOG OF, 6 | 2p16 |
| | HNPCC5 | | |
| CDKN2A | CDKN2 | CYCLIN-DEPENDENT KINASE INHIBITOR 2A | 9p21 |
| | MTS1 | | |
| | p16 | | |
| | MLM | | |
| | CMM2 | | |
| | TP16 | | |
| | p16(INK4) | | |
| | p16(INK4A) | | |
| | p14(ARF) | | |
| | p14arf | | |
| 8q24 | | intergenic | 8q24.21 |
| PRDM2 | RIZ | PR DOMAIN-CONTAINING PROTEIN 2 | 1p36 |
| ABCA4 | ABCR | ATP-BINDING CASSETTE, SUBFAMILY A, | 1p21-p13 |
| | STGD1 | MEMBER 4 | |
| | FEM | | |
| | RP19 | | |
| | CORD3 | | |
| | RMP | | |
| CETP | | CHOLESTERYL ESTER TRANSFER PROTEIN, | 16q21 |
| | | PLASMA | |
| BCHE | CHE1 | BUTYRYLCHOLINESTERASE | 3q26.1-q26.2 |
| DPYD | DPD, | DIHYDROPYRIMIDINE DEHYDROGENASE | 1p22 |
| | DHP | | |
| PI | SERPINA1, | PROTEASE INHIBITOR 1 | 14q32.1 |
| | AAT, | | |
| | PI1 | | |
| CFTR | ABCC7, | CYSTIC FIBROSIS TRANSMEMBRANE | 7q31.2 |
| | CF, | CONDUCTANCE REGULATOR | |
| | MRP7 | | |
| VDR | | VITAMIN D RECEPTOR | 12q12-q14 |
| SRD5A2 | | STEROID 5-ALPHA-REDUCTASE 2 | 2p23 |
| FBN1 | MFS1 | FIBRILLIN 1 | 15q21.1 |
| | WMS | | |
| | FBN | | |
| WFS1 | WFRS | WOLFRAMIN | 4p16.1 |
| | WFS | | |
| | DFNA6 | | |
| ADIPOQ | APM1 | ADIPOCYTE, C1Q, AND COLLAGEN DOMAIN | 3q27 |
| | GBP28 | CONTAINING | |
| | ACRP30 | | |
| INS | | INSULIN | 11p15.5 |
| RET | MEN2A | REARRANGED DURING TRANSFECTION | 10q11.21 |
| | | PROTOONCOGENE | |
| BMP15 | GDF9B, | BONE MORPHOGENETIC PROTEIN 15 | Xp11.2 |
| | ODG2, | | |
| | POF4 | | |
| F9 | HEMB | COAGULATION FACTOR IX | Xq27.1-q27.2 |
| VWF | F8VWF, | COAGULATION FACTOR VIII VWF | 12p13.31 |
| | VWD | | |
| HBB | | HEMOGLOBIN--BETA LOCUS | 11p15.5 |
| FGG | | FIBRINOGEN, G GAMMA POLYPEPTIDE | 4q28 |
| TECTA | DFNA8 | TECTORIN, ALPHA | 11q22-q24 |
| | DFNA12 | | |
| | DFNB21 | | |
| COL3A1 | | COLLAGEN, TYPE III, ALPHA-1 | 2q31 |
| PLOD1 | LLH, | PROCOLLAGEN-LYSINE, 2-OXOGLUTARATE 5- | 1p36.3-p36.2 |
| | LH, | DIOXYGENASE | |
| | LH1, | | |
| | PLOD | | |
| ACCN1 | BNC1 | CATION CHANNEL, AMILORIDE-SENSITIVE, | 17q11.2-q12 |
| | MDEG | NEURONAL, 1 | |
| MSH2 | COCA1, | MutS, E. COLI, HOMOLOG OF, 2 | 2p22-p21 |
| | FCC1, | | |
| | HNPCC1 | | |
| CHEK2 | RAD53, | CHECKPOINT KINASE 2, S. POMBE, HOMOLOG OF | 22q12.1 |
| | CHK2, | | |
| | CDS1, | | |
| | LFS2 | | |
| KLK3 | APS | KALLIKREIN-RELATED PEPTIDASE 3 | 19q13.4 |
| | PSA | | |
| RHO | RP4 | RHODOPSIN | 3q21-q24 |
| | OPN2 | | |
| NYX | CSNB1 | NYCTALOPIN | Xp11.4 |
| CFH | HF1 | COMPLEMENT FACTOR H | 1q32 |
| | HF | | |
| | FHL1 | | |
| | CFHL1 | | |
| | HUS | | |
| CYP2D6 | CYP2D, | CYTOCHROME P450, SUBFAMILY IID, | 22q13.1 |
| | P450C2D, | POLYPEPTIDE 6 | |
| | P450DB1, | | |
| | CPD6 | | |
| CYP2C19 | P450C2C, | CYTOCHROME P450, SUBFAMILY IIC, | 10q24.1-q24.3 |
| | CYP2C | POLYPEPTIDE 19 | |
| CYP2B6 | | CYTOCHROME P450, SUBFAMILY IIB, | 19q13.2 |
| | | POLYPEPTIDE 6 | |
| CYP2C9 | | CYTOCHROME P450, SUBFAMILY IIC, | 10q24 |
| | | POLYPEPTIDE 9 | |
| MLC1 | KIAA0027, | MEGALENCEPHALIC LEUKOENCEPHALOPATHY | 22qter |
| | LVM, | WITH SUBCORTICAL CYSTS GENE 1 | |
| | VL | | |
| WWC1 | KIBRA, | WW, C2, AND COILED-COIL DOMAIN- | 5q34-q35.2 |
| | KIA00869 | CONTAINING 1 | |

The genetic profile (e.g., analysis) can be determined from detecting at least approximately 2, 3, 4, 5, 10, 25, 50, 100, 1000, 2,000, 5000, 6,000, 6,500, 7,000, 8,000, 10,000, 12,000, or 15,000 genetic variants. In some cases, genetic profiles can be determined from at least approximately 20,000, 25,000, 30,000, 45,000, or 50,000 genetic variants. The genetic variants may be SNPs, and each genetic variant may be correlated to a phenotype, such as medically relevant or non-medically relevant phenotypes or conditions. For example, a number of genetic variants (e.g., at least 2, 3, 4, 5, 6, 7, 8, 9, 10, 15, 20, 25, 30, 35, 40, 50, 60, 75, or 100) may cause, be associated with, or be correlated to a single phenotype, or a single genetic variant can be correlated to a single phenotype. A number of genetic variants may also be correlated to a number of phenotypes. Alternatively a single genetic variant may be associated with a number of phenotypes. Each genetic variant can be correlated or associated with at least one phenotype and each phenotype is correlated or associated with at least one genetic variant. For example, a genetic profile may be used to detect (or calculate the risk of, carrier status of, or predisposition for) at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 100, at least 200, or at least 500 phenotypes (e.g., phenotypes described herein). In some cases, a genetic profile is used to detect at least 2 phenotypes, but no more than 10 phenotypes, no more than 15 phenotypes, no more than 20 phenotypes, no more than 25 phenotypes, no more than 30 phenotypes, no more than 35 phenotypes, no more than 40 phenotypes, no more than 45 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1000 phenotypes (e.g., phenotypes described herein). In some cases, a genetic profile is used to detect at least 3 phenotypes, but no more than 10 phenotypes, no more than 20 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1000 phenotypes (e.g., phenotypes described herein). In some cases, a genetic profile is used to detect at least 4 phenotypes, but no more than 10 phenotypes, no more than 20 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1000 phenotypes (e.g., phenotypes described herein). In some cases, a genetic profile is used to detect at least 5 phenotypes, but no more than 10 phenotypes, no more than 20 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1000 phenotypes (e.g., phenotypes described herein). In some cases, a genetic profile is used to detect at least 6 phenotypes, but no more than 10 phenotypes, no more than 20 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1000 phenotypes (e.g., phenotypes described herein). In some cases, a genetic profile is used to detect at least 7 phenotypes, but no more than 10 phenotypes, no more than 20 phenotypes, no more than 50 phenotypes, no more than 100 phenotypes, no more than 200 phenotypes, no more than 300 phenotypes, no more than 500 phenotypes, no more than 1000 phenotypes, or no more than about 10, about 20, about 50, about 100, about 200, about 300, about 500, or about 1006 phenotypes (e.g., phenotypes described herein).

The genetic profiles can also be determined from detecting genetic variants in at least approximately 2, 5, 10, 25, 50, 100, 250, 500, 750, 1000, 1250, 1500, 2000, 2500, 3000, 3500, 4000, 5000, 6000, or genes or loci. In some embodiments, at least approximately 1000, 1500, 2000, 2500, 3000, 4000, 5000 genetic variants are detected in an individual's genetic profile. In some embodiments, approximately 50 or more, 100 or more, 200 or more, 500 or more 1000 or more, 1500 or more, 2000 or more, 2500 or more, 3000 or more, 4000 or more, 5000 or more, or 6000 or more genetic variants are detected in an individual's genetic profile. In some embodiments, at least approximately 6000 genetic variants or at least approximately 6500 genetic variants are detected in an individual's genetic profile. The genetic profile can include genetic variant identification in at least 2, 5, 10, 25, 50, 100, 200, 500, 1000, 1200, 1500, 2000, 3000, 4000, 5000, or 6000 genes. In some embodiments, each of the genetic variants in the genes or loci are associated with one or more phenotypes. In some embodiments, each of the genetic variants in the genes or loci is medically relevant. In some embodiments, each of the sequences is linked to a journal reference or a preventive intervention/recommendation or both. In other embodiments, each of the genetic variants is for a specific disease or for a specific type of genetic testing, such as for children, for a adults, for newborns, for a fetus, for athletes, for carrier information, for cancer patients, transplant recipients or potential transplant recipients, or military recruits.

The genetic variants can also be used to determine the pharmacogenomic profile of an individual and be utilized in assessing clinical trials to stratify the population and further identify genetic variants associated with improved or decreased efficacy or adverse effects. For example, the genetic variants can be used to determine the suitability of a particular medication, drug or treatment for a given disease, condition or phenotype. For example, suitability may include determining whether an individual has a risk of reacting adversely to a drug or treatment, whether a drug may have little effect on the individual's condition (or phenotype), whether a drug is likely to be beneficial to the individual, whether one drug or treatment may be more effective or beneficial than another drug or treatment, whether the drug is likely to be effective in treating a condition, or the timeframe (such as described by a certain number of seconds, minutes, hours, days, weeks, months, years, or decades) in which a response, such as therapeutic response, is likely to be observed with a specific medication or class of medications. Suitability or pharmacogenomics results may include but are not limited to drug resistance, sensitivity, effectiveness, metabolism, absorption, or excretion of a specific drug or class of drugs such as for example aminoglycosides, anti-cancer drugs, sulfonamides, opiates or NSAIDs. Other pharmacogenomic results may include information on a suitable drug dosage for an individual, such as the most appropriate dose of a drug to start at in order to obtain effectiveness or increased effectiveness or to limit potential adverse effects, including but not limited to addiction, toxicity, allergic reaction, abuse potential, treatment-emergent suicidality, hypersensitivity, induced parkinsonism, resistance and intolerance. In some cases, genetic variants are “indicators of” or may be an indicator of which indicates that genetic testing and/or analysis can ascertain one of three possible phenotypes: an increased phenotype, a normal phenotype, or a decreased phenotype. In some cases, genetic variants may provide enhanced protection against an adverse phenotype given a specific intervention. For example, provided herein are variants that indicate hormone therapy may be particularly advantageous for protection against breast cancer.

Non limiting examples of pharmacogenomic genetic variants include variants in cytochrome P450 genes including but not limited to CYP1A1, CYP1A2, CYP1B1, CYP2A6, CYP2A13, CYP2B6, CYP2C8, CYP2C9, CYP2C18, CYP2C19, CYP2D6, CYP2E1, CYP2F1, CYP2J2, CYP2R1, CYP2S1, CYP2W1, CYP3A4, CYP3A5, CYP3A7, CYP3A43, CYP4A11, CYP4A22, CYP4B1, CYP4F2, CYP5A1, CYP8A1, CYP19A1, CYP21A2, CYP26A1, and POR. Other pharmacogenomic related genetic variants in genes or loci include but are not limited to genes or loci for ABC transporters, transporters, methyltransferases, UDP glucuronosyl transferases, lipooxygenases, dehydrogenases, glutathione S transferase, reductases, and oxidoreductases such as for example ABCB1, ABCB11, ABCC1, ABCC2, ABCC4, ABCC8, GSTT1, GSTM1, BDNF, PTGIS, TBXAS1, ORM1, OPRM1, TPH2, FKBP5, UGT1A1, UGT1A2, UGT1A3, UGT1A4, UGT1A5, UGT1A6, UGT1A7, UGT1A8, UGT1A9, UGT1A10, NR1I2, PROZ, APOE, F7, CALU, XRCC3, ADRB2, BMPR2, MTHFR, NPC1L1, GNAS, PROC, EPHX1, GGCX, VKORC1, STX4A, CACNA1S, RYR1, F2, F9, TPMT, NAT1, NAT2, BCHE, ALAD, CDH13, OPRK1, SLC6A4, COQ2, MDM2, PGR, LRP2, HTR2B, RRM1, STAT3, CREB1, CETP, CNR1, ERCC2, SCN1A, SORBS2, CDCA1, FCHSD1, MYO5B, NRG3, LOC644852, EBF3, ATP8B4, GALNTL4, APOA5, APOC3, LEP, AS3MT, ADD2, DCK, MTATP8, HBB, XRCC3, RAD51, HLA-B, MLN, CTLA4, DRD2, KIF6, LDLR, RGS2, UGT1A7, DHFR, HTR3C, BLMH, GPRK5, KIF3A, GSTP1, TNFRSF1B, ABL1, IL10, MAFB, PON1, ARG1, CETP, SLCO1B1, CRHR1, FZD3, SCN2A, FMO2, CINP, NLRC5, ALDH1A1, SERPIND1, CPOX, ODS1, ITPA, DPYD, MTRNR1, HCP5, ADRB1, TNF, GCLM, GCLC, KCNE2, KCNQ1, KCNE1, KCNH2, ITGB3, PON1, ADORA2A, HTR2A, MTCO1, COMT, DRD5, TCF7L2, HMGCR, ADD1, MC1R, SEPP1, PMCH, INPP4B.

Examples of general pharmacogenomic phenotypes that may be tested for and/or analyze include but are not limited to all drugs metabolized by CYP2D6, CYP1A2, CYP1A2, CYP1A2, CYP1A2, CYP2E, CYP2J2, CYP3A7, POR, CYP2C8, CYP3A5, CYP3A7, CYP2B6, CYP1B1, CYP2A6, CYP2A13, CYP2F1, CYP1A1, CYP3A4, CYP1A2, CYP3A43, CYP4A11, CYP4B1, TPMT, CYP8A1, CYP19A1, CYP5A1, CYP2C9, CYP2S1, CYP2C18, CYP2C19, UGT1A6, UGT1A1, UGT1A2, UGT1A3, UGT1A4, UGT1A5, UGT1A6, UGT1A7, UGT1A8, UGT1A9, UGT1A10, NAT1 and/or NAT2.

Examples of specific pharmacogenomic phenotypes that may be tested for and/or analyzed include but are not limited to Increased Metabolism of Oral Opiates (including but not limited to codeine, hydrocodone, oxycodone) to metabolites of increased activity (such as morphine, oxymorphone, or hydromorphone, respectively); Decreased Metabolism of Oral Opiates to metabolites of increased activity; Increased risk of Codeine and/or Oral Opiate Toxicity; No change in risk of Codeine and/or Oral Opiate Toxicity; Decreased risk of Codeine and/or Oral Opiate Toxicity; Tamoxifen Metabolism; Decreased risk of Breast Cancer Relapse with Tamoxifen; Increased risk of Breast Cancer Relapse with Tamoxifen; Increased Effectiveness (Increased Disease Free Survival and/or Decreased Mortality) of Tamoxifen in Treating Breast Cancer; Decreased Effectiveness (Increased Disease Free Survival and/or Decreased Mortality) of Tamoxifen in Treating Breast Cancer; Decreased risk of Adverse Reactions and/or Side Effects (such as Hot Flashes) with Tamoxifen; Increased risk of Adverse Reactions and/or Side Effects with Tamoxifen; Decreased risk of Serious Cardiovascular Events while on Clopidogrel; No change in Serious Cardiovascular Events while on Clopidogrel; Mephenyloin Poor Metabolizer; Mephenyloin Normal Metabolizer; Proguanil Poor Metabolizer; Proguanil Normal Metabolizer; Warfarin Metabolism; Warfarin Resistance; Warfarin Sensitivity; Indicator of Effectiveness of Proton Pump Inhibitors; Indicator of Effectiveness of Antidepressants; Protection against Breast Cancer with Hormone Therapy; Increased risk Breast Cancer with Hormone Therapy; Decreased CYP1A2 Activity in Cigarette Smokers; Increased CYP1A2 Activity in Smokers; Decreased Metabolism of All Drugs Metabolized by CYP1A2 in Cigarette Smokers; Increased Metabolism of All Drugs Metabolized by CYP1A2 in Cigarette Smokers; Poor Clozapine Metabolism; High Blood Levels of Clozapine; Normal Blood Levels of Clozapine; Normal Clozapine Metabolism; Impaired Nicotine Metabolism; Normal Nicotine Metabolism; Protection against Nicotine Addiction; Increased risk of Nicotine Addiction; Poor Metabolism of Tegafur; Normal Metabolism of Tegafur; Impaired Coumarin Metabolism; Normal Coumarin Metabolism; Reduced Sensitivity to Xenobiotic Toxicity; Reduced Risk of Xenobiotic Toxicity; Normal Sensitivity to Xenobiotic Toxicity; Normal Risk of Xenobiotic Toxicity; Increased Sensitivity to Xenobiotic Toxicity; Increased risk of Xenobiotic Toxicity; Increased risk of Sporadic Amyotrophic Lateral Sclerosis due to Exposure to Metal and/or Solvent/Chemicals; No Increased risk of Sporadic Amyotrophic Lateral Sclerosis due to Exposure to Metal and/or Solvent/Chemicals; Increased Likelihood of Buproprion Effectiveness for Successful Treatment of Nicotine Addiction (such as Abstinence from Nicotine at 10 Weeks and/or Six Months); Decreased Likelihood of Buproprion Effectiveness for the Successful Treatment of Nicotine Addiction; Impaired Efavirenz Metabolism; Normal Efavirenz Metabolism; Increased Plasma Concentration of Efavirenz; Normal Plasma Concentration of Efavirenz; Increased risk of Side Effects (such as Central Nervous System-related Side Effects) with Efavirenz; Decreased risk of Side Effects with Efavirenz; Reduced Dosage Required with Efavirenz for Therapeutic Effect; Normal Dosage Required with Efavirenz for Therapeutic Effect; Reduced Starting Dosage of Efavirenz Required for Therapeutic Effect; Normal Starting Dosage of Efavirenz Required for Therapeutic Effect; Cyclophosphamide Metabolism; Bupropion Metabolism; Increased risk of Statin-induced Rhabdomyolysis; Protection against Statin-induced Rhabdomyolysis; Poor Paclitaxel Metabolism; Normal Paclitaxel Metabolism; Reduced Arachidonic Acid Metabolism; Normal Arachidonic Acid Metabolism; Reduced Linoleic Acid Metabolism; Normal Linoleic Acid Metabolism; Indicator of Dose of Tacrolimus Required for Renal Transplant Patients; Increased CYP3A7 Enzyme Activity; Normal CYP3A7 Enzyme Activity; Decreased CYP3A7 Enzyme Activity; Improved Treatment Efficacy (Delayed Disease Progression) of Aromatase Inhibitors (such as Letrozole) in Breast Cancer (such as Advanced Breast Carcinoma); Warfarin Sensitivity; Warfarin Resistance; Warfarin Metabolism; Phenyloin Poor Metabolizer; Phenyloin Normal Metabolizer; Decreased Effectiveness of Phenyloin; Normal Effectiveness of Phenyloin; Glipizide Poor Metabolizer; Glipizide Normal Metabolizer; Decreased Effectiveness of Glipizide; Normal Effectiveness of Glipizide; Impaired Diclofenac Metabolism; Normal Diclofenac Metabolism; Decreased Effectiveness of Diclofenac; Normal Effectiveness of Diclofenac; Diphenylhydantoin Toxicity; Protection against Diphenylhydantoin Toxicity; Bitumen Metabolism; Increased risk of Bitumen Toxicity; Normal Risk of Bitumen Toxicity; Increased Time (such as Median Time) to First INR within Therapeutic Range with Warfarin Normal Starting Dose; Normal Time (such as Median Time) to First INR within Therapeutic Range with Warfarin Normal Starting Dose; Decreased Time (such as Median Time) to First INR within Therapeutic Range with Warfarin Normal Starting Dose; Increased Time to First INR >4 with Warfarin Normal Starting Dose; Normal Time to First INR >4 with Warfarin Normal Starting Dose; Decreased Time to First INR >4 with Warfarin Normal Starting Dose; Higher Mean Maintenance Dose Of Warfarin Needed for Therapeutic Anticoagulation; Normal Mean Maintenance Dose Of Warfarin Needed for Therapeutic Anticoagulation; Lower Mean Maintenance Dose Of Warfarin Needed for Therapeutic Anticoagulation; Malignant Hyperthermia with Anesthesia (such as General Anesthesia, Volatile Anesthetics, Gas Anesthetics, and/or Succinylcholine); TPMT Deficiency; 6-Mercaptopurine Sensitivity; Increased risk of 6-Mercaptopurine Toxicity; Protection against 6-Mercaptopurine Toxicity; Increased risk of Azathioprine Toxicity; Protection against Azathioprine Toxicity; 6-Mercaptopurine-induced Myelosuppression; Protection against 6-Mercaptopurine-induced Myelosuppression; Azathioprine-induced Myelosuppression; Protection against Azathioprine-induced Myelosuppression; Severe Hematologic Toxicity after Mercaptopurine; Protection against Severe Hematologic Toxicity after Mercaptopurine; Slow Acetylation by NAT1; Normal Acetylation by NAT1; Fast Acetylation by NAT1; Slow Acetylation by NAT2; Normal Acetylation by NAT2; Fast Acetylation by NAT2; Postanesthetic Apnea (Such as from Anesthesia and/or Muscle Relaxants, Including but Not Limited to Suxamethonium); Fluoride-resistant Butyrylcholinesterase; Dibucaine-resistant Butyrylcholinesterase; Susceptibility to Lead Poisoning; Protection against Lead Poisoning; Decreased Effectiveness of Opiates (including but not limited to morphine, heroin, codeine, hydrocodone, oxycodone, oxymorphone, hydromorphone, dihydromorphine, and/or any derivative of opium, morphine or codeine) in Treating Pain (Analgesic Effect); Normal Effectiveness of Opiates in Treating Pain (Analgesic Effect); Decreased Response to Opiates Requiring Larger Dosages for Analgesic Effect; Normal Response to Opiates Requiring Normal Dosages for Analgesic Effect; Decreased risk of Opioid-induced Respiratory Depression; Increased risk of Opioid-induced Respiratory Depression; Susceptibility to Opioid Dependence; Protection against Opioid Dependence; Decreased Breast Cancer Relapse with Tamoxifen; No Effect on Breast Cancer Relapse with Tamoxifen; Indicator of Effectiveness of Clopidogrel; Decreased risk of Death from Cardiovascular Causes, Myocardial Infarction, and/or Stroke while on Clopidogrel; No Change in Risk of Death from Cardiovascular Causes, Myocardial Infarction, and/or Stroke while on Clopidogrel; Decreased risk of Death from Cardiovascular Causes while on Clopidogrel; No Change in risk of Death from Cardiovascular Causes while on Clopidogrel; Decreased risk of Stent Thrombosis while on Clopidogrel; No Change in risk of Stent Thrombosis while on Clopidogrel; Bitumen Metabolism (Sensitivity or Resistance to Occupational Exposure to Bitumen); Indicator of Eating Cruciferous Vegetables confers Protection against Myocardial Infarction; Indicator of Eating Cruciferous Vegetables may not confer Protection against Myocardial Infarction; Protection against Lung Cancer with the Consumption of Cruciferous Vegetables or Cabbage, Broccoli and Brussels Sprouts at least Once a Week; No Protection or Insignificant Protection against Lung Cancer with the Consumption of Cruciferous Vegetables or Cabbage, Broccoli and Brussels Sprouts at least Once a Week; Protection against Lung Cancer with the Consumption of Cruciferous Vegetables or Cabbage, Broccoli and Brussels Sprouts at least Once a Week; No Protection or Insignificant Protection against Lung Cancer with the Consumption of Cruciferous Vegetables or Cabbage, Broccoli and Brussels Sprouts at least Once a Week; Cue-induced Craving for Alcohol; Protection against Cue-induced Craving for Alcohol; Improved Survival (such as 2-Year Survival) with Temozolomide to Treat Glioblastoma; No Effect of Temozolomide in Prolonging Survival (such as 2-year Survival) during treatment of Glioblastoma; Poor Clinical Response to SSRIs (including but not limited to citalopram, dapoxetine, escitalopram, fluoxetine, fluvoxamine, paroxetine, sertraline, and/or zimelidine) to Treat Depression; Normal Clinical Response to SSRIs to Treat Depression; Digoxin, Higher Plasma Levels; Digoxin, Normal Plasma Levels; Colchicine Resistance; Decreased Response to Colchicine; Normal Response to Colchicine; Lower Methadone Dose (such as <150 mg/day) Needed for Effective Treatment of Heroin Dependence; Higher Methadone Dose (such as >150 mg/day) Needed for Effective Treatment of Heroin Dependence; Cyclosporin A, Lower Bioavailability of; Cyclosporin A, Normal Bioavailability of; HIV-1 Protease Inhibitors, Lower Bioavailability; HIV-1 Protease Inhibitors, Normal Bioavailability; Drug-resistant Epilepsy; Protection against Insecticide Exposure Increasing the Risk of Childhood Leukemia (such as Childhood Acute Lymphoblastic Leukemia); No Protection against Insecticide Exposure Increasing the Risk of Childhood Leukemia (such as Childhood Acute Lymphoblastic Leukemia); Decreased Uptake of Orally Administered P-glycoprotein (PGP) Substrates; Normal Uptake of Orally Administered P-glycoprotein (PGP) Substrates; Increased Uptake of Orally Administered P-glycoprotein (PGP) Substrates; Myocardial Protection with Beta Blockers (Including but Not Limited to Bucindolol) During Heart Failure; No or Insignificant Myocardial Protection with Beta Blockers During Heart Failure; Decreased Mortality with Beta Blockers During Heart Failure; No Decreased Mortality with Beta Blockers During Heart Failure; Increased Therapeutic Response to Beta Blockers During Heart Failure; Decreased Therapeutic Response to Beta Blockers During Heart Failure; Increased risk of Persistent Bone Marrow Dysplasia following Chronic Exposure to Benzene; Protection against Persistent Bone Marrow Dysplasia following Chronic Exposure to Benzene; Stronger Response (Mitsuda Reaction) to Lepromin; Normal Response (Mitsuda Reaction) to Lepromin; Drug-induced (Including but Not Limited to Sulfamethoxazole, Clarithromycin, Dofetilide, Quinidine, Sotolol, Amiodarone, Haloperidol, Ziprasidone, and/or Cisapride) Long QT Syndrome; Protection against Drug-induced Long QT Syndrome; Drug-induced Long QT Interval; Protection against Drug-induced Long QT Interval; Drug-induced (Including but Not Limited to Sulfamethoxazole, Clarithromycin, Dofetilide, Quinidine, Sotolol, Amiodarone, Haloperidol, Ziprasidone, and/or Cisapride) Torsade de pointes; Protection against Drug-induced Torsade de pointes; Drug-induced Torsade de pointes; Protection against Drug-induced Torsade de pointes; Impaired Antithrombotic Action of Acetylsalicylic acid; Normal Antithrombotic Action of Acetylsalicylic acid; Decreased Antithrombotic Effectiveness of Acetylsalicylic acid; Normal Antithrombotic Effectiveness of Acetylsalicylic acid; No Decreased Risk of Cardiovascular Events (Including but Not Limited to Myocardial Infarction) with Acetylsalicylic acid; Decreased Risk of Cardiovascular Events with Acetylsalicylic acid; No Decreased Risk of Neurologic Events (Including but Not Limited to Transient Ischemic Attack and/or Ischemic Stroke) with Acetylsalicylic acid; Decreased Risk of Neurologic Events with Acetylsalicylic acid; Decreased Metabolism of Chemical Weapons of Mass Destruction (Including but Not Limited to Sarin Nerve Gas); Normal Metabolism of Chemical Weapons of Mass Destruction; Increased Sensitivity to Chemical Weapons of Mass Destruction; No Increased Sensitivity to Chemical Weapons of Mass Destruction; Increased Sensitivity to Pesticide-based Weapons of Mass Destruction, such as Insecticides, Herbicides, Solvents, Plasticizers, and Extreme Pressure Additives (such as Diazinon); No Increased Sensitivity to Pesticide-based Weapons of Mass Destruction (Including but not Limited to Organophosphates, such as Insecticides, Herbicides, Solvents, Plasticizers, and Extreme Pressure Additives (such as Diazinon)); Sensitivity to Caffeine; No Increased Sensitivity to Caffeine; Increased risk of Reduced Sleep Quality due to Caffeine Consumption; Decreased Risk of Reduced Sleep Quality due to Caffeine Consumption; Increased risk of Insomnia due to Caffeine Consumption; Decreased Risk of Insomnia due to Caffeine Consumption; No Reduced Sensitivity to Citalopram; Reduced Sensitivity to Citalopram; Reduced Effectiveness of Citalopram; No Reduced Sensitivity to Clozapine; Reduced Sensitivity to Clozapine; Reduced Effectiveness of Clozapine; Increased Absorption of MDR-1 Substrates; Normal Absorption of MDR-1 Substrates; Decreased Absorption of MDR-1 Substrates; Increased Tissue Concentrations of MDR-1 Substrates; Normal Tissue Concentrations of MDR-1 Substrates; Decreased Tissue Concentrations of MDR-1 Substrates; Indicator of Rifampin-induced P-glycoprotein Levels; Atypical Porphyrinogenic Response to Mercury; Increased risk of Mercury Toxicity; Protection against Mercury Toxicity; Increased risk of Azathioprine Toxicity; Protection against Azathioprine Toxicity; Increased risk of 5-Fluorouracil Toxicity; Protection against 5-Fluorouracil Toxicity; Sensitivity to 5-Fluorouracil; No Sensitivity to 5-Fluorouracil; Increased Effectiveness of the Therapeutic Response of Antidepressant Drugs (Such as SSRIs, Including but not Limited to Citalopram) in Treating Depression (such as Major Depressive Disorder) with Citalopram; Decreased Effectiveness of the Therapeutic Response of Antidepressant Drugs in Treating Depression with Citalopram; No Effect of Amphetamines on Augmenting Cognition; Detrimental Effect of Amphetamines on Cognition; Positive Effects of Amphetamines on Cognition; Increased risk of Adverse Effects from Amphetamines; Protection against Adverse Effects from Amphetamines; Successful Treatment with Metyrosine of Neuropsychiatric Illness associated with 22q11.2 Deletion Syndrome; Metyrosine Useful to Treat the Neuropsychiatric Illness associated with 22q11.2 Deletion Syndrome; Metyrosine Not Useful to Treat the Neuropsychiatric Illness associated with 22q11.2 Deletion Syndrome; Decreased Binding of Risperidone; Normal Binding of Risperidone; Depression Poorly Responsive to SSRIs; Depression Unresponsive to SSRIs; Accelerated Response Time to Antidepressant Drug Treatment in Depression (Faster Onset of Therapeutic Effects of Antidepressants in Treating Depression); Normal Response Time to Antidepressant Drug Treatment in Depression (Normal Onset of Therapeutic Effects of Antidepressants in Treating Depression); Toxicity of Irinotecan; Protection against Toxicity of Irinotecan; Severe Toxicity of Irinotecan; Protection against Severe Toxicity of Irinotecan; Lower Starting Dose of Irinotecan; Normal Starting Dose of Irinotecan; Abnormal Laboratory Values (such as Increased Bilirubin Levels) with Tranilast; No Abnormal Laboratory Values with Tranilast; Increased risk of Hepatic Complications (such as Liver Damage) with Tranilast; Protection against Hepatic Complications with Tranilast; Resistance to Mitomycin-C; No Resistance to Mitomycin-C; Indicator of Effectiveness of Mitomycin-C; Decreased Bronchodilator (e.g. Beta Agonists, including but not limited to P2-agonists Albuterol, Levalbuterol, Fenoterol, Formoterol, Isoproterenol, Metaproterenol, Salmeterol, Terbutaline, and/or Clenbuterol) Therapeutic Response in Treating Asthma; Increased Bronchodilator (such as Beta Agonists) Therapeutic Response in Treating Asthma; Positive Long Term Response of Asthma to Albuterol Use; Decreasing Long Term Response of Asthma to Albuterol Use; Increased Therapeutic Response in Treating Asthma with the Withdrawal from Beta-agonist Therapy and Replacement with Ipratropium Bromide; No Therapeutic Benefit in Treating Asthma with the Withdrawal from Beta-agonist Therapy and Replacement with Ipratropium Bromide; Asthma Worsened with Beta Agonists; Asthma Improved with Beta Agonists; Decreased Vasodilation with β2-agonists; Normal Vasodilation with β2-agonists; Increased Vasoconstriction with β2-agonists; Normal Vasoconstriction with β2-agonists; Decreased Vasoconstriction with β2-agonists; Nonresponse to Ezetimibe; Normal Response to Ezetimibe; Decrease Effectiveness of Beta Blocker Therapy to Treat Hypertension; Normal Effectiveness of Beta Blocker Therapy to Treat Hypertension; Increased Effectiveness of Beta Blocker Therapy to Treat Hypertension; Decreased Effectiveness of Sulfonylureas in Treating Diabetes Mellitus, Type II; Normal Effectiveness of Sulfonylureas in Treating Diabetes Mellitus, Type II; Decreased Effectiveness of Statins (e.g. HMG-CoA reductase inhibitors, including but not limited to Atorvastatin, Cerivastatin, Fluvastatin, Lovastatin, Mevastatin, Pitavastatin, Pravastatin, Rosuvastatin, and/or Simvastatin) in Reducing Total Cholesterol and/or LDL Cholesterol Levels; Normal Effectiveness of Statins in Reducing Total Cholesterol and/or LDL Cholesterol Levels; Increased risk of Coronary Heart Disease with Diuretic Use as Compared to ACE Inhibitors or Calcium Channel Blockers in Treating Hypertension; No Increased risk of Coronary Heart Disease with Diuretic Use as Compared to ACE Inhibitors or Calcium Channel Blockers in Treating Hypertension; ACE Inhibitors or Calcium Channel Blockers May be Better Choice in Treating Hypertension as Compared to Diuretics due to Increased Risk of Coronary Heart Disease with Diuretics but Not with ACE Inhibitors or Calcium Channel Blockers; No contraindications to Using Diuretics To Treat Hypertension; Increased Dose of Anesthesia Required for Anesthetic Effects; Normal Dose of Anesthesia Required for Anesthetic Effects; Increased Analgesia Effects from Opioids (such as κ-opioid); Normal Analgesia Effects from Opioids (such as K-opioid); Decreased Analgesia Effects from Opioids (such as κ-opioid); Indicator of Selenoprotein Levels; Selenium Metabolism; Increased Selenoprotein Levels after Selenium Supplementation; Normal Selenoprotein Levels after Selenium Supplementation; Decreased Selenoprotein Levels after Selenium Supplementation; Increased risk of Obesity with Antipsychotics (Including but Not Limited to Olanzapine); Protection against Obesity with Antipsychotics (Including but Not Limited to Olanzapine); Daunorubicin-Induced Toxicity (such as Cytotoxicity); No Increased Risk of Daunorubicin-Induced Toxicity (such as Cytotoxicity); Cisplatin-Induced Toxicity (such as Cytotoxicity); No Increased Risk of Cisplatin-Induced Toxicity (such as Cytotoxicity); Increased risk of Opiate Addiction; No Increased risk of Opiate Addiction; Indicator of Effectiveness of Therapeutic Response to SSRIs in Treating Depression; Indicator of Adverse Effects with SSRIs; Increased risk of Adverse Drug Reactions (such as Severe Adverse Events) with Antidepressants (Including but Not Limited to SSRIs and/or Mirtazapine); Protection against Adverse Drug Reactions with Antidepressants (Including but Not Limited to SSRIs and/or Mirtazapine); Lower Starting Dose of Antidepressants (Including but Not Limited to SSRIs and/or Mirtazapine) Required to Limit Side-Effects and/or Adverse Drug Reactions; Lower Final Daily Doses of Antidepressants (Including but Not Limited to SSRIs and/or Mirtazapine) Required for Therapeutic Response in Treating Depression and/or to Limit Side-effects and/or Adverse Drug Reactions; Normal Final Daily Doses of Antidepressants (Including but Not Limited to SSRIs and/or Mirtazapine) Required for Therapeutic Response in Treating Depression; Higher Final Daily Doses of Antidepressants (Including but Not Limited to SSRIs and/or Mirtazapine) Required for Therapeutic Response in Treating Depression; Increased risk of Discontinuations with SSRIs (such as due to Adverse Events); No Increased risk of Discontinuations (such as due to Adverse Events) with Antidepressants (Including but Not Limited to SSRIs and/or Mirtazapine); Fewer Discontinuations (such as Due to Adverse Events) with Antidepressants (Including but Not Limited to SSRIs and/or Mirtazapine); Increased risk of New or Worsening Suicidal Ideation during Short-term Treatment with Antidepressants (Including but Not Limited to SSRIs); Protection against New or Worsening Suicidal Ideation during Short-term Treatment with Antidepressants (Including but Not Limited to SSRIs); Increased risk of Treatment-Emergent Suicidality during Treatment with Antidepressants (Including but not limited to SSRIs); Protection against Treatment-Emergent Suicidality during Treatment with Antidepressants (Including but not limited to SSRIs); Decreased Survival (Increased Mortality) with Beta Agonists (such as P2-agonists) When Used to Treat Congestive Heart Failure; Normal Survival (Normal Mortality) with Beta Agonists (such as β2-agonists) When Used to Treat Congestive Heart Failure Decreased Survival (Increased Mortality) with β2-agonists When Used to Treat Congestive Increased Survival (Decreased Mortality) with Beta Agonists (such as P2-agonists) When Used to Treat Congestive Heart Failure; Dexfenfluramine-associated Primary Pulmonary Arterial Hypertension; Fenfluramine-associated Primary Pulmonary Hypertension; Fen-Phen-associated Primary Pulmonary Arterial Hypertension; Increased Sensitivity to Nitrous Oxide; Normal Sensitivity to Nitrous Oxide; Increased risk of Nitrous Oxide Toxicity; Protection against Nitrous Oxide Toxicity; Decreased Dose of Nitrous Oxide Required for Therapeutic Effect; Normal Dose of Nitrous Oxide Required for Therapeutic Effect; Increased B-vitamin Nutritional Supplementation Requirements; No Increased B-vitamin Nutritional Supplementation Requirements; Increased risk of Methotrexate Induced Alopecia; Protection against of Methotrexate Induced Alopecia; Impaired Methotrexate Elimination; Normal Methotrexate Elimination; Increased risk of Methotrexate Toxicity; Protection against Methotrexate Toxicity; Increased risk of Side-Effects with Methotrexate; Protection against Side-Effects with Methotrexate; Lower Dose of Methotrexate Required; Normal Dose of Methotrexate Required; Longer Time until Discontinuation (such as >20 Months) of Methotrexate Likely Due to Toxicity and/or Side-effects; Shorter Time until Discontinuation (such as <6 Months) of Methotrexate Likely Due to Toxicity and/or Side-effects; Longer Time until Decrease of Dose (such as >20 Months) of Methotrexate Needed Due to Toxicity and/or Side-effects; Shorter Time until Decrease of Dose (such as <6 Months) of Methotrexate Needed Due to Toxicity and/or Side-effects; Decreased Effectiveness of Lithium in Treating Bipolar Disorder; Normal Effectiveness of Lithium in Treating Bipolar Disorder; Decreased Analgesic Effectiveness of Opiates; Normal Analgesic Effectiveness of Opiates; Increased Dose of Opiates Required for Analgesic Effect; Normal Dose of Opiates Required for Analgesic Effect; Decreased Opioid-induced Respiratory Depression; Normal Opioid-induced Respiratory Depression; Increased risk of Opioid Dependence; Protection against Opioid Dependence; Increased Effectiveness of Naltrexone in Treating Alcoholism, Alcohol Abuse, and/or Alcohol Dependence; Normal Effectiveness of Naltrexone in Treating Alcoholism, Alcohol Abuse, and/or Alcohol Dependence; Decreased Effectiveness of Naltrexone in Treating Alcoholism, Alcohol Abuse, and/or Alcohol Dependence; Stronger Effect of Naltrexone in Blunting Alcohol-induced Highs; Normal Effect of Naltrexone in Blunting Alcohol-induced Highs; Weaker Effect of Naltrexone in Blunting Alcohol-induced Highs; Increased risk of Nicotine Addiction; Protection against Nicotine Addiction; Increased Reinforcing Value of Nicotine; Normal Reinforcing Value of Nicotine; Decreased Reinforcing Value of Nicotine; Increased Chance of being a Remitter after a Single Antidepressant (such as SSRIs) Treatment for Mood Disorder; Decreased Chance of being a Remitter after a Single Antidepressant (such as SSRIs) Treatment for Mood Disorder; Increased Effectiveness of Antidepressants (such as SSRIs) in Treating Mood Disorders; Increased Effectiveness of Fluoxetine in Treating Anxiety; Normal Effectiveness of SSRIs in Treating Anxiety; Decreased Effectiveness of SSRIs in Treating Anxiety; Increased Effectiveness of SSRIs in Treating Anxiety Associated with Stressful Situations; Normal Effectiveness of SSRIs in Treating Anxiety Associated with Stressful Situations; Decreased Effectiveness of SSRIs in Treating Anxiety Associated with Stressful Situations; Increased risk of Side-effects (Including but Not Limited to Myositis, Myopathy, and/or Rhabdomyolysis) with Statins; Resistance to Topoisomerase II-Targeting Chemotherapeutic Drugs (Including but Not Limited to Etoposide, Mitoxantrone, Amsacrine, and Ellipticine); No Resistance to Topoisomerase II-Targeting Chemotherapeutic Drugs (Including but Not Limited to Etoposide, Mitoxantrone, Amsacrine, and Ellipticine); Decreased Effectiveness of Topoisomerase II-Targeting Chemotherapeutic Drugs (Including but Not Limited to Etoposide, Mitoxantrone, Amsacrine, and Ellipticine); Normal Effectiveness of Topoisomerase II-Targeting Chemotherapeutic Drugs (Including but. Not Limited to Etoposide, Mitoxantrone, Amsacrine, and Ellipticine); Insensitivity to Mifepristone; Decreased Effectiveness of Mifepristone; Normal Effectiveness of Mifepristone; Increased risk of Adverse Reactions (Including but Not Limited to Ototoxic Effects) of Cisplatin; No Increased risk of Adverse Reactions (Including but Not Limited to Ototoxic Effects) of Cisplatin; Increased risk of Gemcitabine-Induced Neutropenia; Protection against Gemcitabine-Induced Neutropenia; Resistance to Gemcitabine; No Resistance to Gemcitabine; Decreased Effectiveness of Gemcitabine; Normal Effectiveness of Gemcitabine; Increased Effectiveness of Interferon Alpha in Treating Patients with Metastatic Renal Cell Carcinoma; Normal Effectiveness of Interferon Alpha in Treating Patients with Metastatic Renal Cell Carcinoma; Decreased Effectiveness of Interferon Alpha in Treating Patients with Metastatic Renal Cell Carcinoma; Increased Effectiveness of Statins in Slowing Progression of Coronary Atherosclerosis; Normal Effectiveness of Statins in Slowing Progression of Coronary Atherosclerosis; Decreased Effectiveness of Statins in Slowing Progression of Coronary Atherosclerosis; Increased risk of Cardiovascular Disease Events in Statin-Treated Familial Hypercholesterolemia; Protection against Cardiovascular Disease Events in Statin-Treated Familial Hypercholesterolemia; Cardiovascular Disease Events in Statin-Treated Familial Hypercholesterolemia; Sudden Death in People with Diabetes Mellitus, Type II; Elevated HDL Cholesterol Levels (that can be Diminished with Higher Triglyceride Levels); Increased Effectiveness of Antipsychotics (Including but Not Limited to Risperidone, Haloperidol, Olanzapine, and/or Clozapine) in Treating Schizophrenia; Normal Effectiveness of Antipsychotics (Including but Not Limited to Risperidone, Haloperidol, Olanzapine, and/or Clozapine) in Treating Schizophrenia; Decreased Effectiveness of Antipsychotics (Including but Not Limited to Risperidone, Haloperidol, Olanzapine, and/or Clozapine) in Treating Schizophrenia; Increased risk of Arsenic-induced Precancer and/or Cancer (such as Premalignant Hyperkeratosis); Protection against Arsenic-induced Precancer and/or Cancer (such as Premalignant Hyperkeratosis); Increased Survival with Resected Gastric Cancer Treated with Chemo-radiotherapy; No Increased Survival with Resected Gastric Cancer Treated with Chemo-radiotherapy; Increased Cholesterol Levels with First Generation Antipsychotics (Including but not Limited to Haloperidol, Fluphenazine, Molindone, Thiothixene, Thioridazine, Trifluoperazine, Loxapine, Perphenazine, Prochlorperazine, Pimozide, and Zuclopenthixol) and Lower Cholesterol Levels with Olanzapine and/or Clozapine; Less Chance of Olanzapine and/or Clozapine Increasing Cholesterol Levels as Opposed to First Generation Antipsychotics (Including but not Limited to Haloperidol, Fluphenazine, Molindone, Thiothixene, Thioridazine, Trifluoperazine, Loxapine, Perphenazine, Prochlorperazine, Pimozide, and Zuclopenthixol); No Increased Cholesterol Levels with First Generation Antipsychotics (Including but not Limited to Haloperidol, Fluphenazine, Molindone, Thiothixene, Thioridazine, Trifluoperazine, Loxapine, Perphenazine, Prochlorperazine, Pimozide, and Zuclopenthixol); Decreased Triglyceride Levels with Antipsychotics (Including but Not Limited to Olanzapine or Clozapine); No Change in Triglyceride Levels with Antipsychotics (Including but Not Limited to Olanzapine or Clozapine); Increased Triglyceride Levels with Antipsychotics (Including but Not Limited to Olanzapine or Clozapine); Increased Weight Gain (such as After 9 Months) While on Antipsychotics (Including but Not Limited to Olanzapine); No Weight Gain (such as After 9 Months) While on Antipsychotics (Including but Not Limited to Olanzapine); Increased risk of Olanzapine-induced Weight Gain; Protection against Olanzapine-induced Weight Gain; Increased Arsenic Methylation (Increased Urinary Excretion of Monomethylarsonic Acid); Normal Arsenic Methylation (Normal Urinary Excretion of Monomethylarsonic Acid); Increased Risk for Toxic (such as Genotoxic) Effects of Arsenic Exposure; Normal Risk for Toxic (such as Genotoxic) Effects of Arsenic Exposure; Increased Effectiveness of Blood Pressure in Lowering Blood Pressure in Hypertensives as Compared to Diuretics; No Increased Effectiveness of Blood Pressure in Lowering Blood Pressure in Hypertensives as Compared to Diuretics; Lower Blast ara-CTP Levels in AML Patients Receiving ara-C as Continuous Infusion; No Change in Blast ara-CTP Levels in AML Patients Receiving ara-C as Continuous Infusion; Increased Effectiveness of ara-C as Continuous Infusion in Treating AML Patients; Normal Effectiveness of ara-C as Continuous Infusion in Treating AML Patients; Lower Effectiveness of ara-C as Continuous Infusion in Treating AML Patients; Increased risk of Valproate-induced Reversible Brain Pseudoarthropathy; Protection against Valproate-induced Reversible Brain Pseudoarthropathy; Aminogycloside-induced Deafness; Protection against Aminogycloside-induced Deafness; Lower Dose of Antiepileptic Medication (Including but Not Limited to Carbamazepine and/or Phenyloin) Needed to Control Epileptic Symptoms (such as Seizures); Normal Dose of Antiepileptic Medication (Including but Not Limited to Carbamazepine and/or Phenyloin) Needed to Control Epileptic Symptoms (such as Seizures); Higher Dose of Antiepileptic Medication (Including but Not Limited to Carbamazepine and/or Phenyloin) Needed to Control Epileptic Symptoms (such as Seizures); Maximum Dose of Carbamazepine Needed to Control Epilepsy approximately 1,313 mg/day; Maximum Dose of Carbamazepine Needed to Control Epilepsy approximately 1,225 mg/day; Maximum Dose of Carbamazepine Needed to Control Epilepsy approximately 1,083 mg/day; Maximum Dose of Phenyloin Needed to Control Epilepsy approximately 373 mg/day; Maximum Dose of Phenyloin Needed to Control Epilepsy approximately 340 mg/day; Maximum Dose of Phenyloin Needed to Control Epilepsy approximately 326 mg/day; Increased risk of Persistent Bone Marrow Dysplasia following Chronic Exposure to Benzene; Protection against Persistent Bone Marrow Dysplasia following Chronic Exposure to Benzene; Increased Cholesterol Levels with Risperidone; No Increased Cholesterol Levels with Risperidone; Increased risk of Antiviral (Such as Reverse Transcriptase Inhibitors Including but Not Limited to Abacavir) Hypersensitivity; Protection against Antiviral (Such as Reverse Transcriptase Inhibitors Including but Not Limited to Abacavir) Hypersensitivity; Increased risk of Adverse Reactions with Antivirals (Such as Reverse Transcriptase Inhibitors Including but Not Limited to Abacavir); Protection against Adverse Reactions with Antivirals (Such as Reverse Transcriptase Inhibitors Including but Not Limited to Abacavir); Drug-induced (Including but Not Limited to Sulfonamides such as Acetazolamide, Benzolamide, Bumetanide, Celecoxib, Chlorthalidone, Clopamide, Dichlorphenamide, Dorzolamide, Ethoxzolamide, Furosemide, Hydrochlorothiazide, Indapamide, Mafenide, Mefruside, Metolazone, Probenecid, Sulfacetamide, Sulfadiazine, Sulfadimethoxine, Sulfadoxine, Sulfanilamides, Sulfamethoxazole, Trimethoprim-sulfamethoxazole (Co-trimoxazole), Sulfamethoxypyridazine, Sulfasalazine, Sultiame, Sumatriptan, Xipamide, and/or Zonisamide) Hemolysis; Increased risk of Adverse Reactions (such as Thrombotic Events) with Valproic Acid; No Increased risk of Adverse Reactions (such as Thrombotic Events) with Valproic Acid; Improved Survival with Childhood Acute Myelogenous Leukemia when Treated with Medications That Generate DNA Double-strand Breaks (Including but Not Limited to Etoposide and/or Daunomycin) as Compared to Treatment with Anti-metabolites (Including but Not Limited to Fludarabine and/or Cytarabine); No Improved Survival with Childhood Acute Myelogenous Leukemia when Treated with Medications That Generate DNA Double-strand Breaks (Including but Not Limited to Etoposide and/or Daunomycin) as Compared to Treatment with Anti-metabolites (Including but Not Limited to Fludarabine and/or Cytarabine); Increased risk of Chemotherapy-related Adult Leukemia (such as Adult Acute Myelogenous Leukemia); Protection against Chemotherapy-related Adult Leukemia (such as Adult Acute Myelogenous Leukemia); Increased risk of Mitomycin-C Resistance; Protection against Mitomycin-C Resistance; Increased risk of Adverse Reactions (Including but Not Limited to Stevens-Johnson Syndrome and/or Hypersensitivity Syndrome) with Carbamazepine; Protection against Adverse Reactions (Including but Not Limited to Stevens-Johnson Syndrome and/or Hypersensitivity Syndrome) with Carbamazepine; Increased risk of Adverse Reactions (Including but Not Limited to Severe Cutaneous Reaction) with Allopurinol; Protection against Adverse Reactions (Including but Not Limited to Severe Cutaneous Reaction) with Allopurinol; Increased risk of Cyclosporine-induced Gingival Overgrowth; Protection against Cyclosporine-induced Gingival Overgrowth; Increased Effectiveness of CTLA-4 Blockade for the Treatment of Melanoma; Normal Effectiveness of CTLA-4 Blockade for the Treatment of Melanoma; Decreased Effectiveness of CTLA-4 Blockade for the Treatment of Melanoma; Increased Effectiveness (such as Smoking Cessation) of Bupropion Treatment for Nicotine Addiction; Decreased Effectiveness (such as Smoking Cessation) of Bupropion Treatment for Nicotine Addiction; Increased Likelihood of Abstinence from Cigarette Smoking after Buproprion Treatment; Decreased Likelihood of Abstinence from Cigarette Smoking after Buproprion Treatment; Longer Time to Therapeutic Response with Antipsychotics during First Episode of Schizophrenia; Shorter Time to Therapeutic Response with Antipsychotics during First Episode of Schizophrenia; Increased Effectiveness of High-dose (such as 80 mg) Atorvastatin Therapy in Reducing the Risk of Death and/or Major Cardiovascular Events as Compared with Standard-dose Pravastatin Therapy; No Increased Effectiveness of High-dose (such as 80 mg) Atorvastatin Therapy in Reducing the Risk of Death and/or Major Cardiovascular Events as Compared with Standard-dose Pravastatin Therapy; No Benefit from High-dose Atorvastatin compared with Standard-dose Pravastatin Therapy; Reduced risk of Coronary Heart Disease with Pravastatin; No Reduced risk of Coronary Heart Disease with Pravastatin; Reduced risk of Cardiovascular Events with Statins (such as Pravastatin); Increased risk of Cardiovascular Events on Statins (such as Pravastatin); Increased Effectiveness of Statins (such as Pravastatin) in Lowering LDL Levels; No Increased Effectiveness of Statins (such as Pravastatin) in Lowering LDL Levels; Increased risk of Methotrexate Toxicity; Protection against Methotrexate Toxicity; Increased risk of Antipsychotic-induced (Including but Not Limited to Risperidone, Olanzapine, and/or Clozapine) Parkinsonism; Protection against Antipsychotic-induced (Including but Not Limited to Risperidone, Olanzapine, and/or Clozapine) Parkinsonism; Increased risk of Irinotecan Toxicity; Protection against Irinotecan Toxicity; Chemotherapy-induced Vomiting, Acute; Increased risk of Methotrexate Resistance; Protection against Methotrexate Resistance; Increased risk of Early Relapse after Chemotherapy (such as Bleomycin-containing Chemotherapy) to Treat Testicular Cancer (such as Testicular Germ Cell Cancer); Protection against risk of Early Relapse after Chemotherapy (such as Bleomycin-containing Chemotherapy) to Treat Testicular Cancer (such as Testicular Germ Cell Cancer); Decreased Survival (Increased Mortality) with Bleomycin-containing Chemotherapy to Treat Testicular Cancer (such as Testicular Germ Cell Cancer); Increased Survival (Decreased Mortality) with Bleomycin-containing Chemotherapy to Treat Testicular Cancer (such as Testicular Germ Cell Cancer); Decreased Mortality in Heart Failure When Treated with Beta Blockers; No Decrease in Mortality in Heart Failure When Treated with Beta Blockers; Decreased Effectiveness of Beta Blockers in Treating Heart Failure; Increased Effectiveness of Beta Blockers in Treating Heart Failure; Increased risk of Penicillin Allergy; Protection against Penicillin Allergy; Testosterone Doping May not Be Detected by a Drug Screen; Testosterone Doping Will be Detectable on a Drug Screen; Indicator of Urinary Testosterone/Epitestosterone Ratio Needed in order to Detect Testosterone Doping (Increases Sensitivity and Decreases False Positives of Drug Screen); Increased risk of Oxaliplatin-related Adverse Reaction (such as Neuropathy); Protection against Oxaliplatin-related Adverse Reaction (such as Neuropathy); Increased Survival (Decreased Mortality) with Metastatic Colorectal Cancer Being Treated with Chemotherapy (such as 5-fluorouracil/oxaliplatin); No Change in Survival (No Change in Mortality) with Metastatic-Colorectal Cancer Being Treated with Chemotherapy (such as 5-fluorouracil/oxaliplatin); Decreased Survival (Increased Mortality) with Metastatic Colorectal Cancer Being Treated with Chemotherapy (such as 5-fluorouracil/oxaliplatin); Increased Survival with Metastatic Colorectal Cancer Being Treated with Chemotherapy (such as 5-fluorouracil/oxaliplatin); Decreased Effectiveness of Infliximab Therapy in Treating Autoimmune Disease (Including but Not Limited to Psoriasis, Crohn Disease, Ankylosing Spondylitis, Psoriatic Arthritis, Rheumatoid Arthritis, Sarcoidosis and/or Ulcerative Colitis); Increased Effectiveness of Infliximab Therapy in Treating Autoimmune Disease (Including but Not Limited to Psoriasis, Crohn Disease, Ankylosing Spondylitis, Psoriatic Arthritis, Rheumatoid Arthritis, Sarcoidosis and/or Ulcerative Colitis); Increased Thiopurine Sensitivity; Decreased Thiopurine Sensitivity; Increased Effectiveness of Sulfonylurea (Including but Not Limited to Gliclazide) to Treat Diabetes Mellitus, Type II; Decreased Effectiveness of Sulfonylurea (Including but Not Limited to Gliclazide) to Treat Diabetes Mellitus, Type II; Resistance to Imatinib; Sensitivity to Imatinib; Increased Effectiveness of anti-TNF Treatment for Rheumatoid Arthritis; Decreased Effectiveness of anti-TNF Treatment for Rheumatoid Arthritis; Increased risk of Being a Non-Responder to anti-TNF Treatment for Rheumatoid Arthritis; Protection against Being a Non-Responder to anti-TNF Treatment for Rheumatoid Arthritis; Increased risk of Stroke with Statins; Decreased risk of Stroke with Statins; Increased risk of Statin-induced Myopathy; Protection against Statin-induced Myopathy; Increased risk of Statin-induced Myositis; Protection against Statin-induced Myositis; Increased risk of Statin-induced Rhabdomyolysis; Protection against Statin-induced Rhabdomyolysis; Increased Effectiveness of Inhaled Corticosteroids for Treatment of Asthma; Decreased Effectiveness of Inhaled Corticosteroids for Treatment of Asthma; Increased risk of Methamphetamine Psychosis; Protection against Methamphetamine Psychosis; Increased risk of Antiepileptic Drug (such as Carbamazapine and/or Phenyloin) Resistance; Protection against Antiepileptic Drug (such as Carbamazapine and/or Phenyloin) Resistance; Decreased Effectiveness of Antiepileptic Drugs (such as Carbamazapine and/or Phenyloin) in Treating Epilepsy and/or Seizures; Normal Effectiveness of Antiepileptic Drugs (such as Carbamazapine and/or Phenyloin) in Treating Epilepsy and/or Seizures; Increased Effectiveness of Antiepileptic Drugs (such as Carbamazapine and/or Phenyloin) in Treating Epilepsy and/or Seizures; Increased risk of Adverse Reactions (such as Pulmonary Toxicity) when Exposed to Thioureas; Protection against Adverse Reactions (such as Pulmonary Toxicity) when Exposed to Thioureas; Increased risk of Adverse Reactions (such as Venous Thromboembolism) with Thalidomide; Protection against Adverse Reactions (such as Venous Thromboembolism) with Thalidomide; Decreased Subjective Effects of Alcohol with Finasteride; No Decreased Subjective Effects of Alcohol with Finasteride; Effectiveness of Finasteride in Treating Alcoholism, Alcohol Abuse, and/or Alcohol Dependence, Indicator of; and Determination of Best Treatment Protocol for Alcoholism, Alcohol Abuse, and/or Alcohol Dependence, such as Determining Most Effective Medication Treatment (such as Finasteride or Naltrexone) and/or Most Effective 12-Step Program (such as Twelve-step Facilitation Program, Cognitive Behavioral Therapy, or Motivational Enhancement Therapy).

The evaluation of the genetic variants and their relationship to phenotype and the significance to the client may be further analyzed to produce one of a variety of scores that combine two or more of the variants identified and in some embodiments also include non-genetic information about the client to provide a score as described herein. The particular profile or score provided in the report to the client to third party may be based on a request from the client, doctor or another third party as described herein.

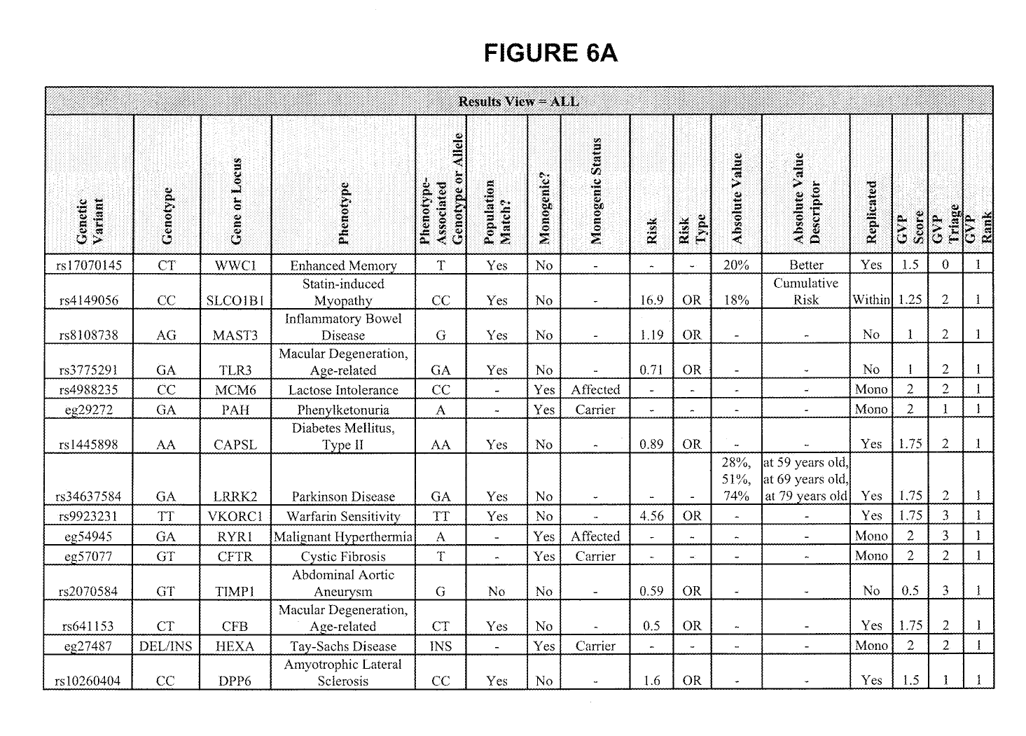

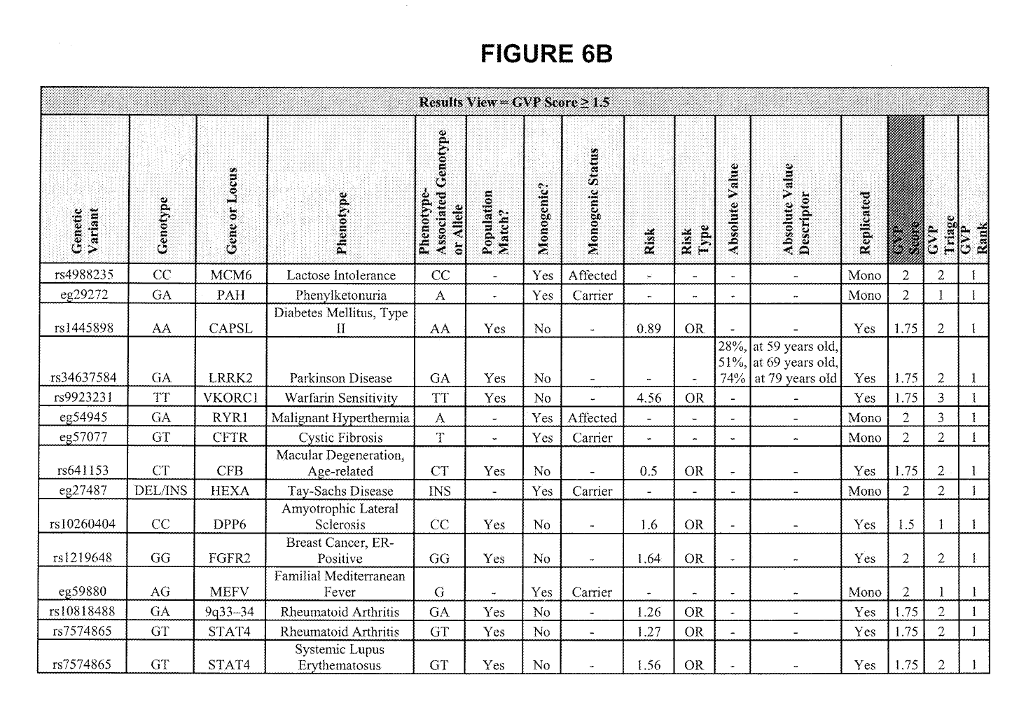

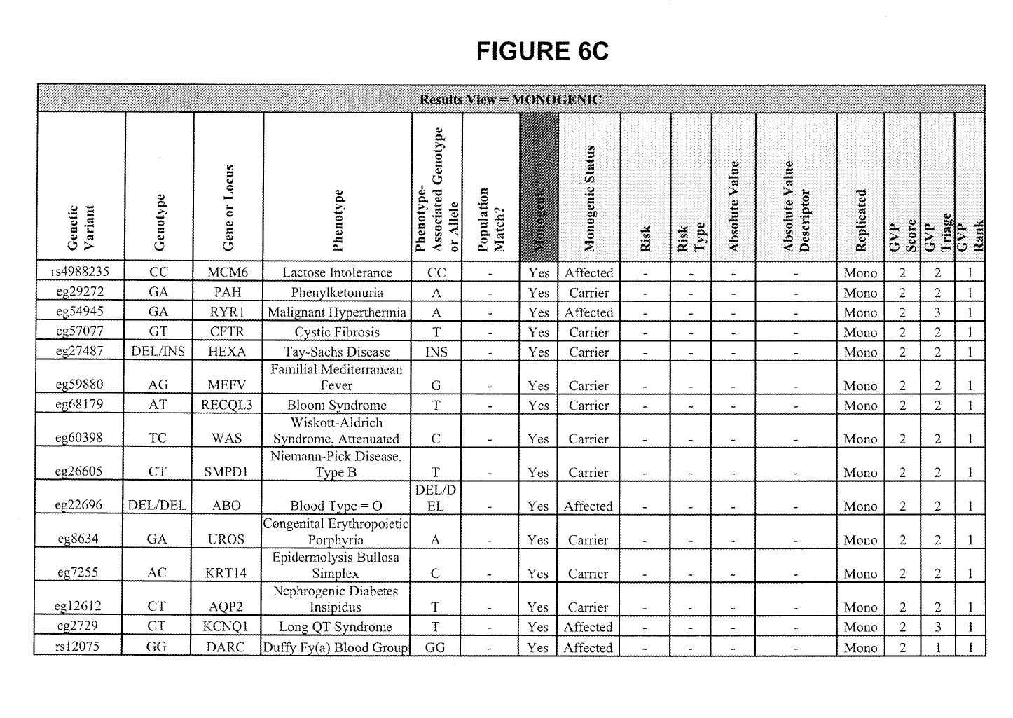

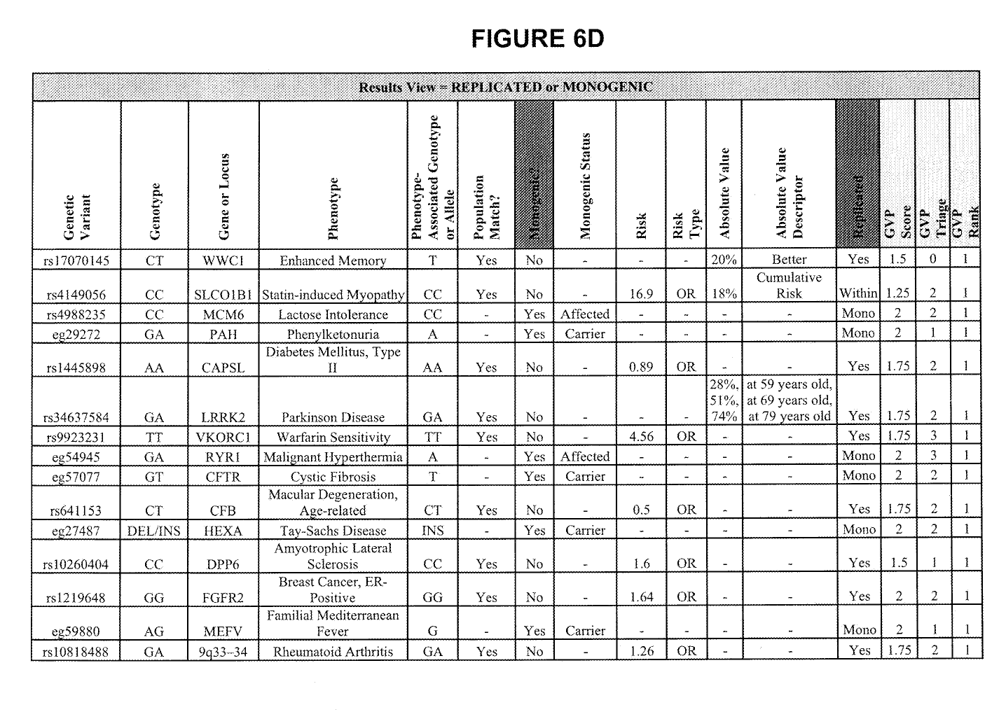

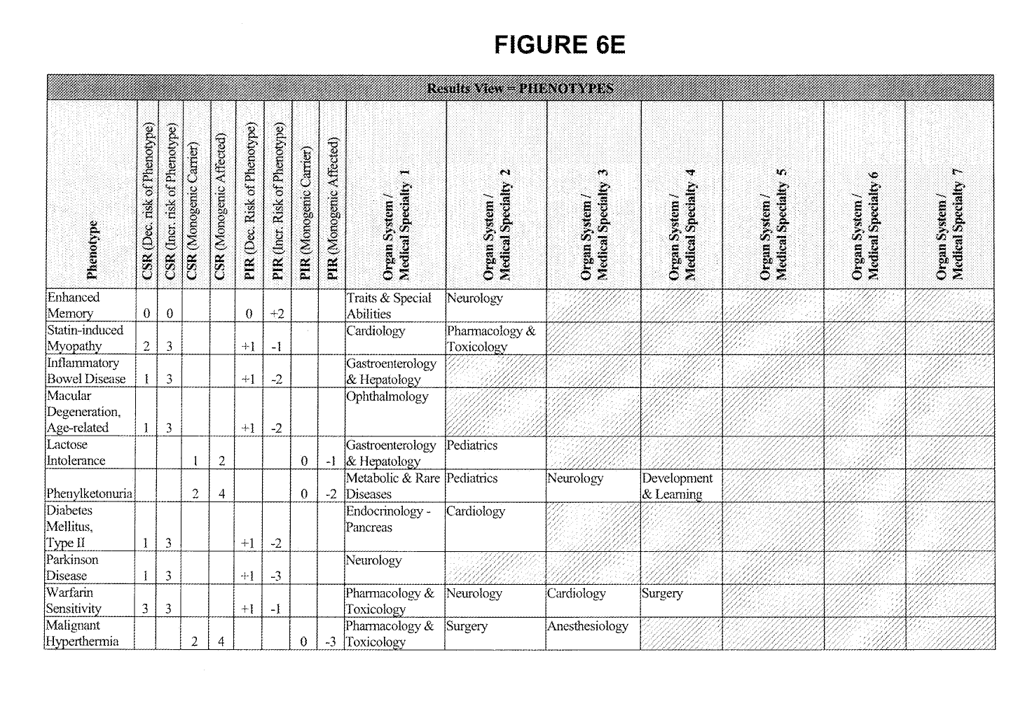

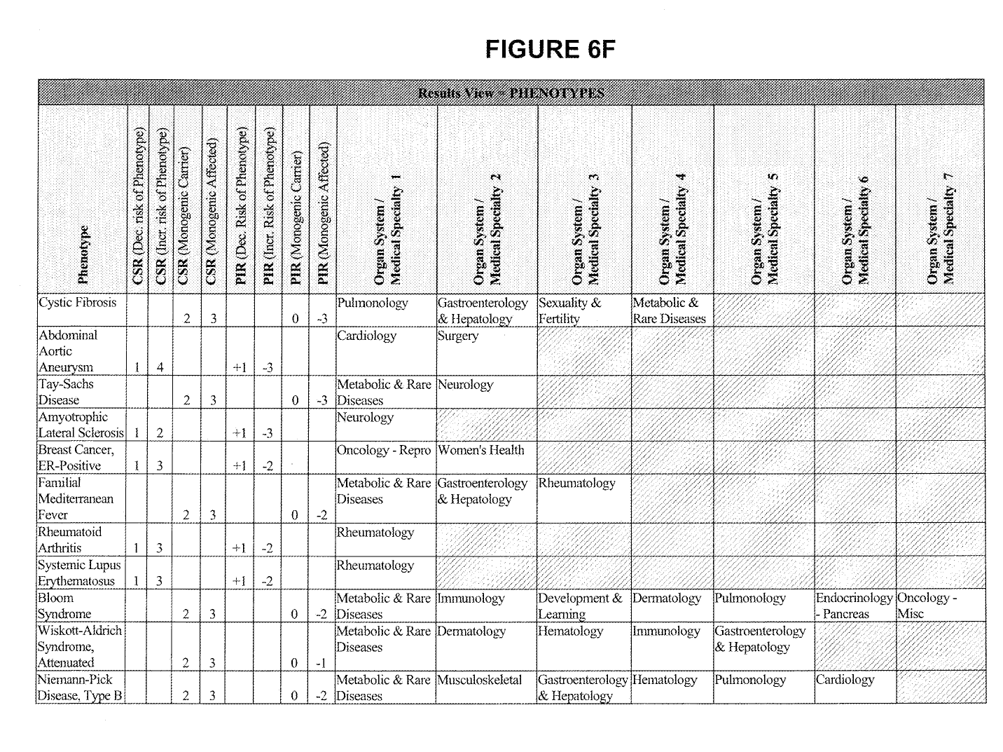

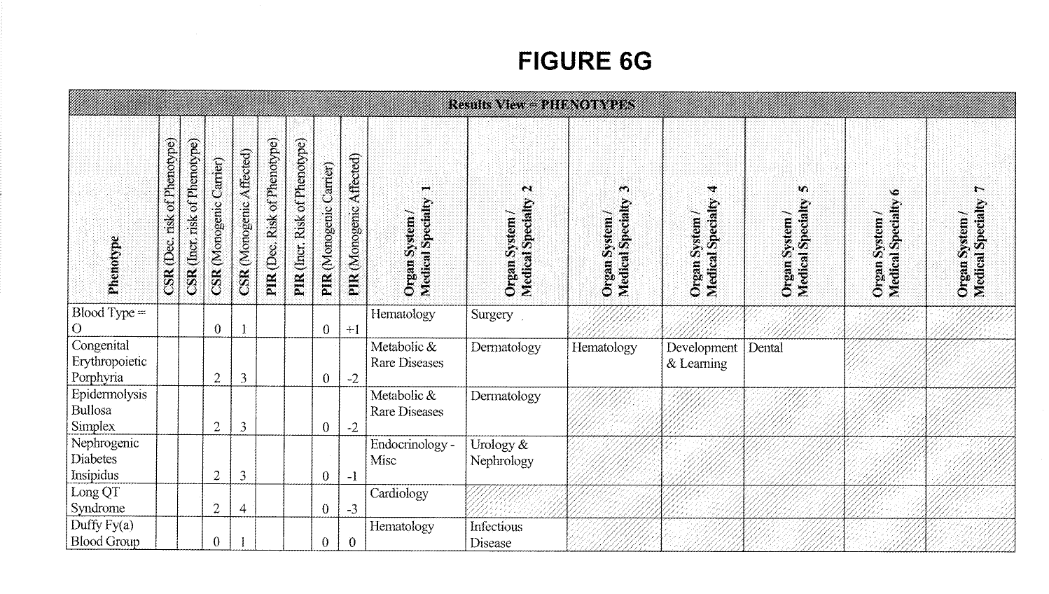

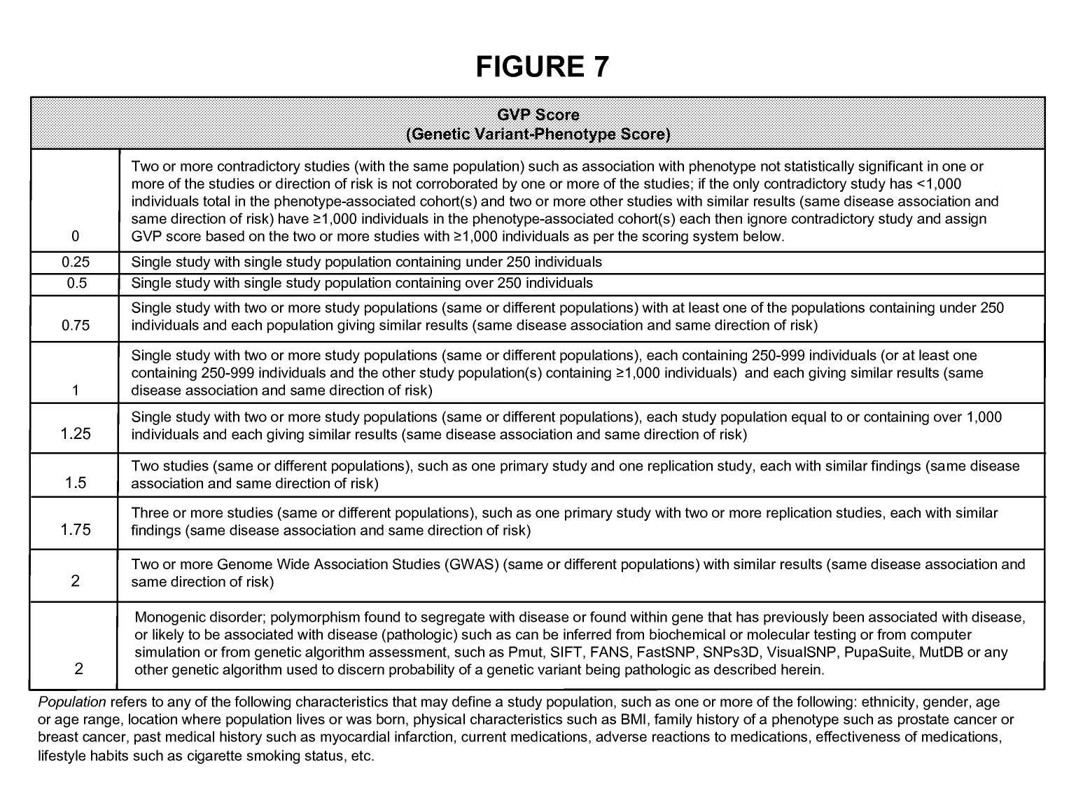

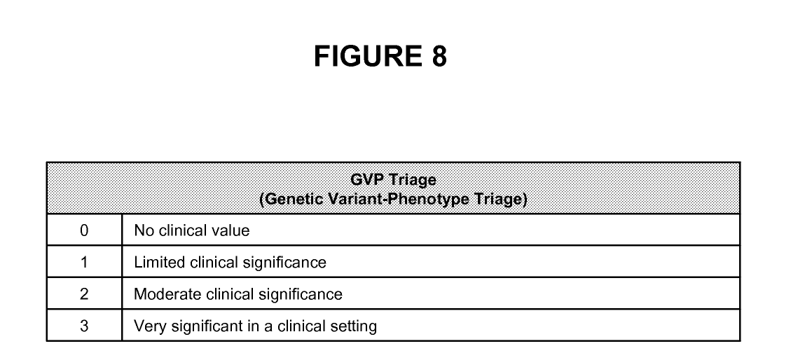

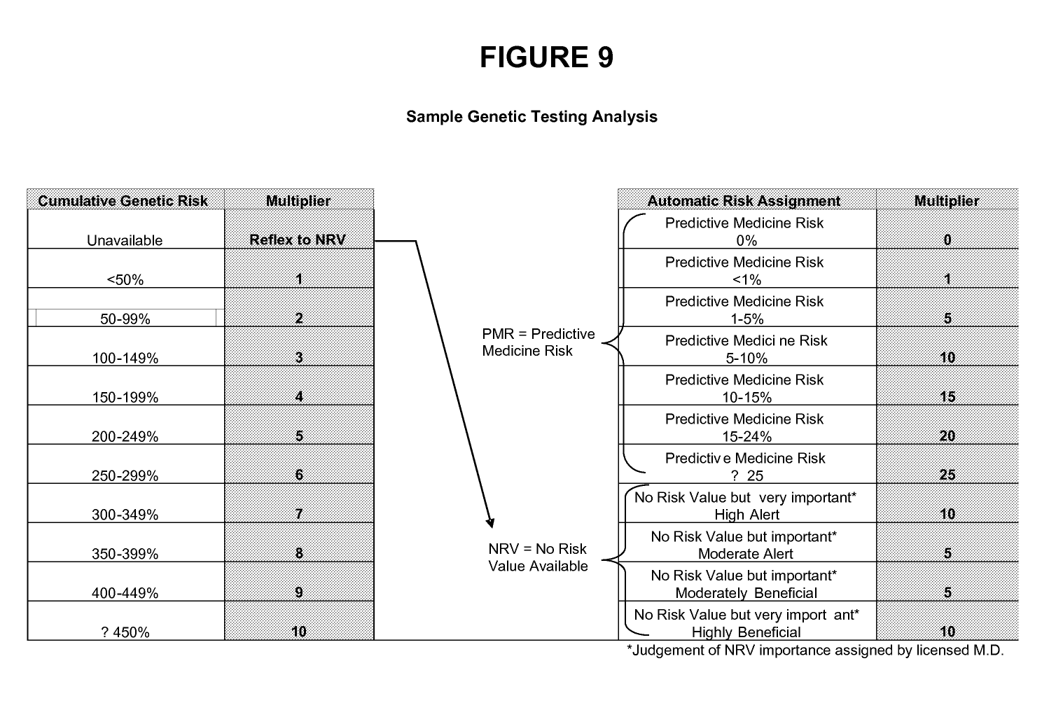

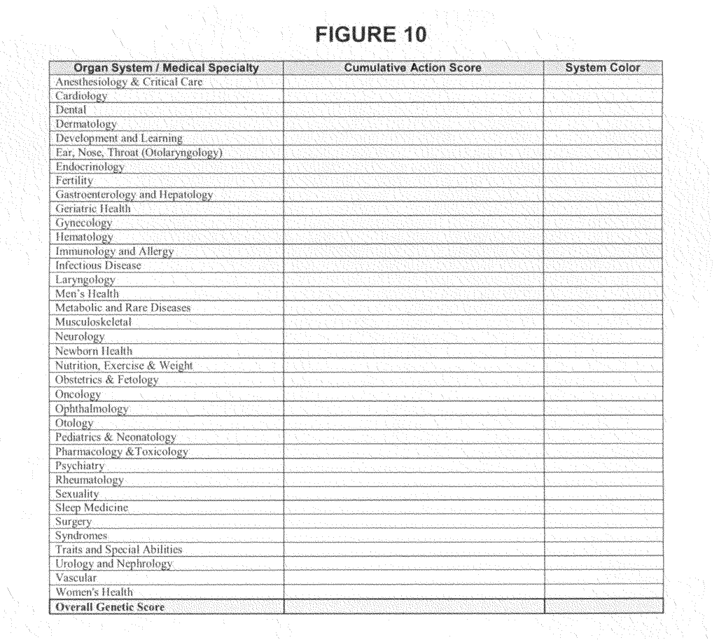

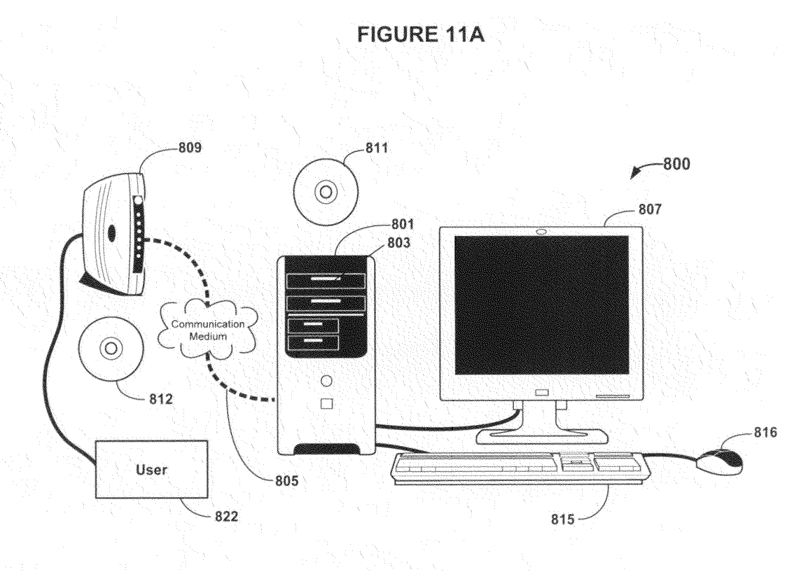

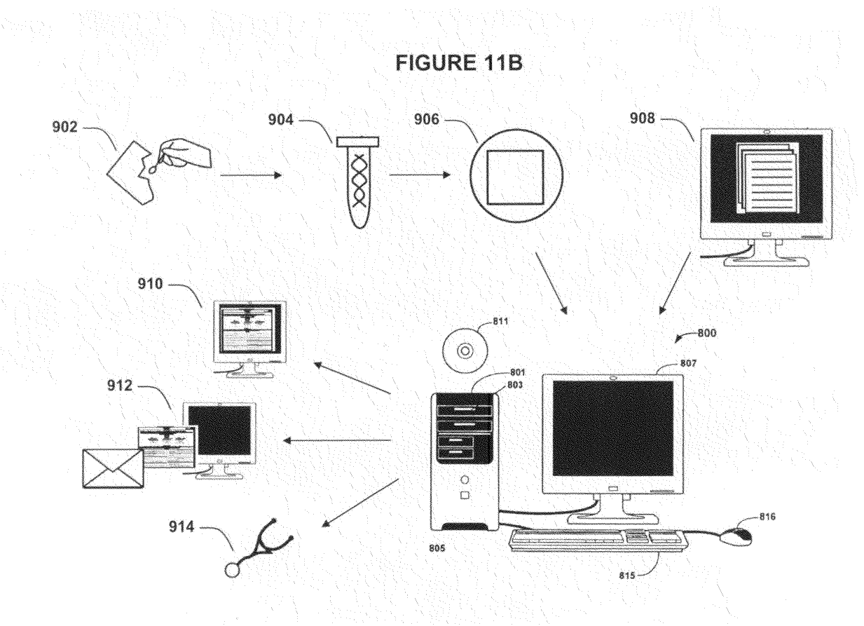

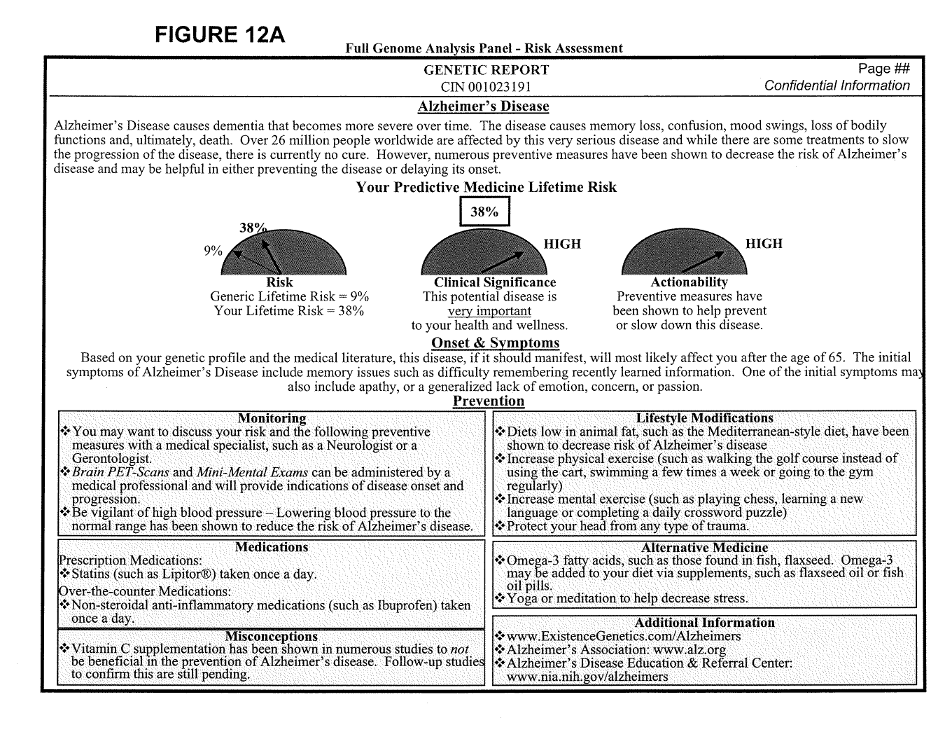

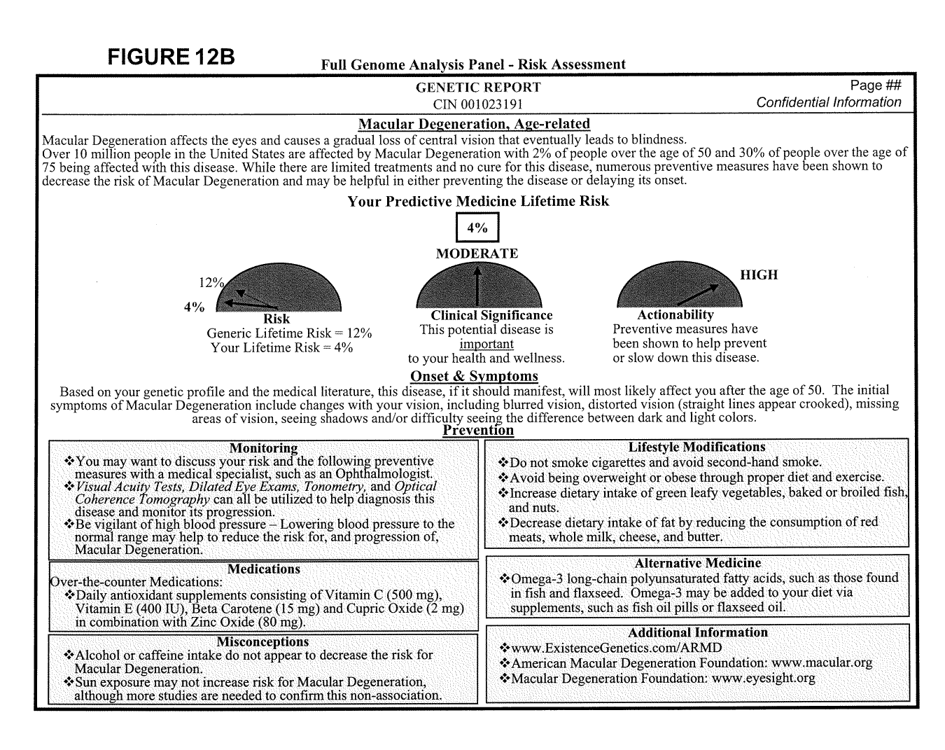

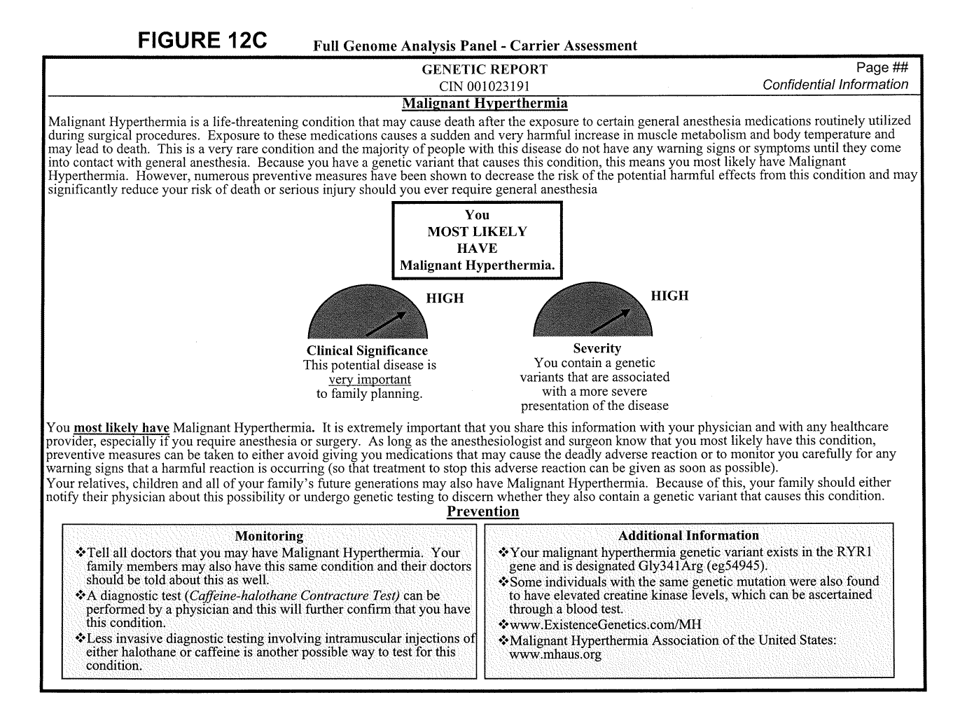

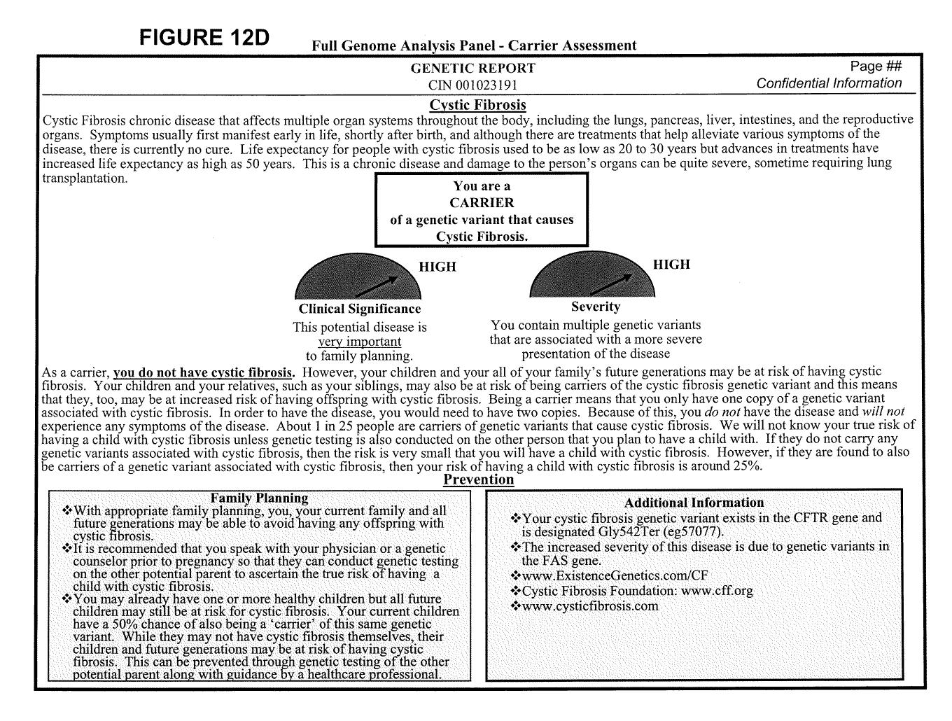

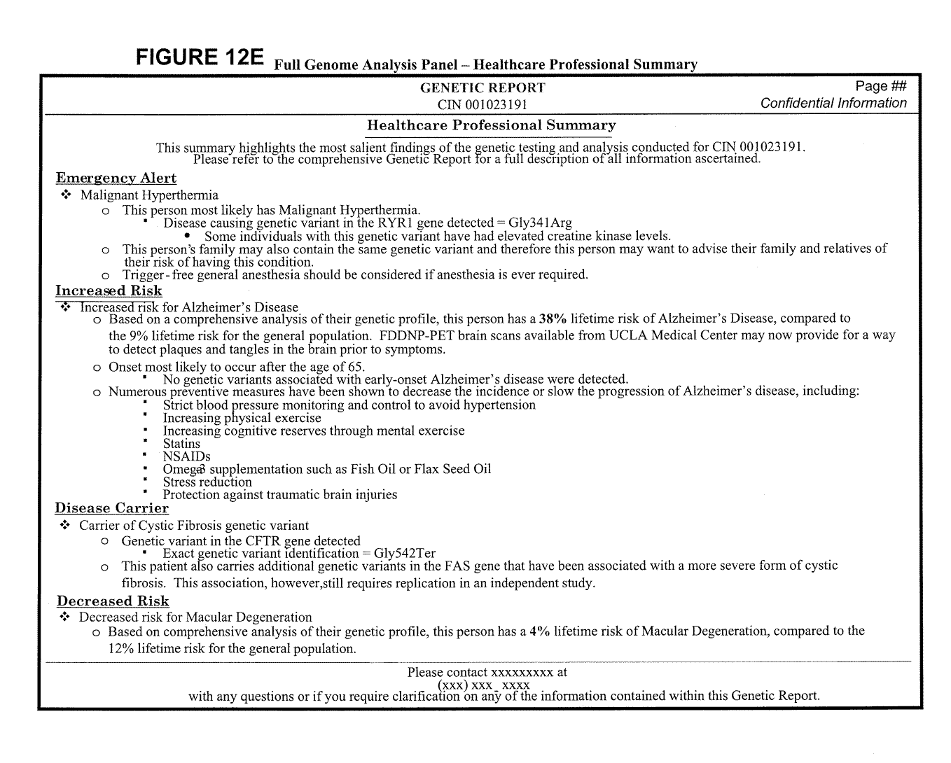

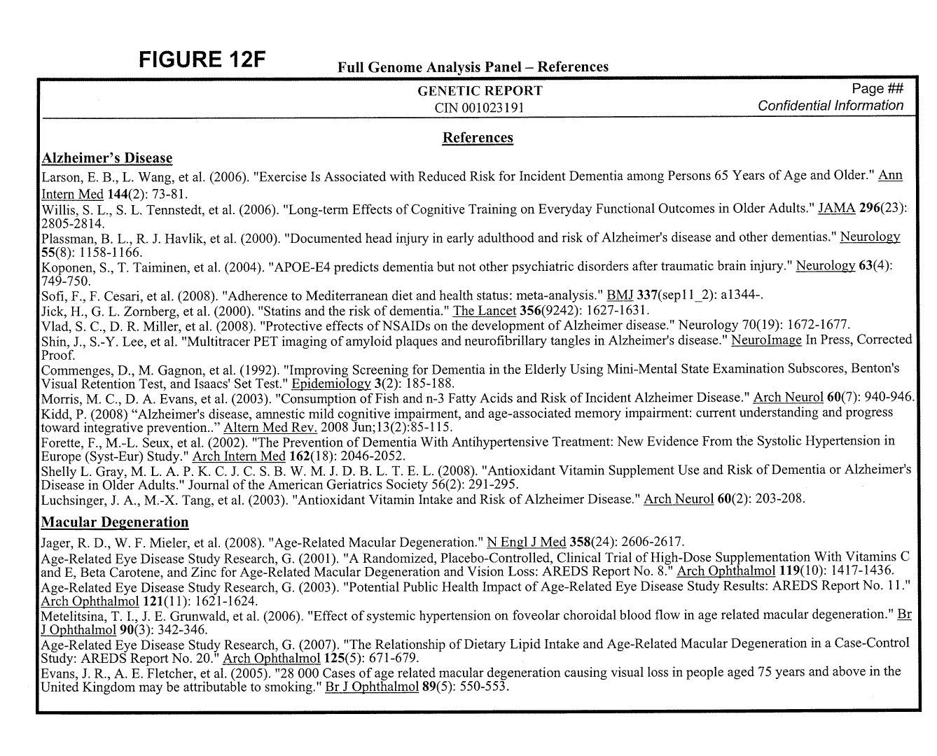

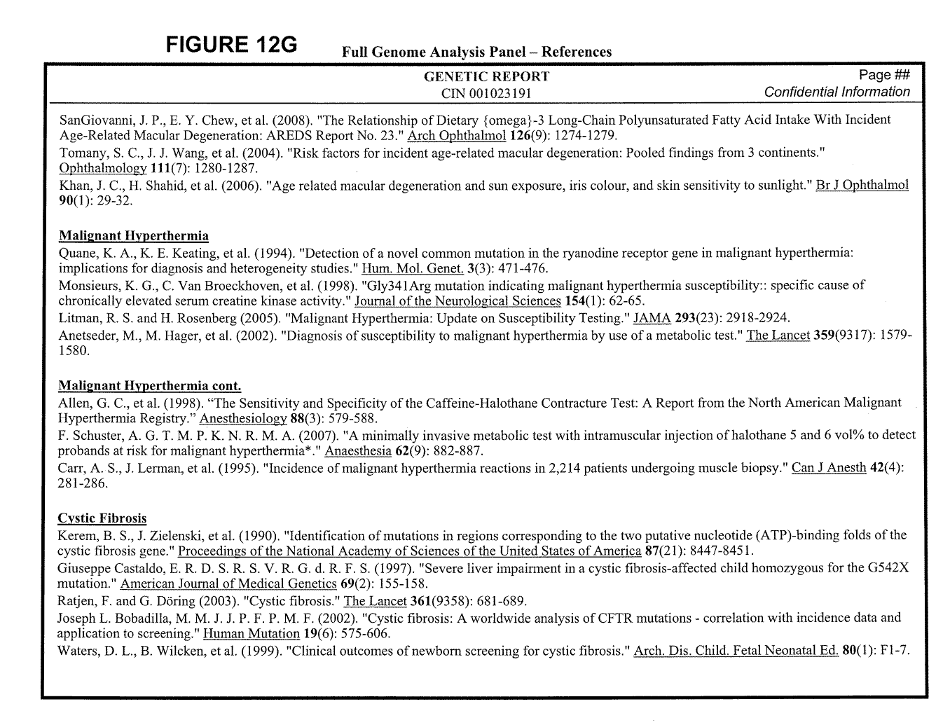

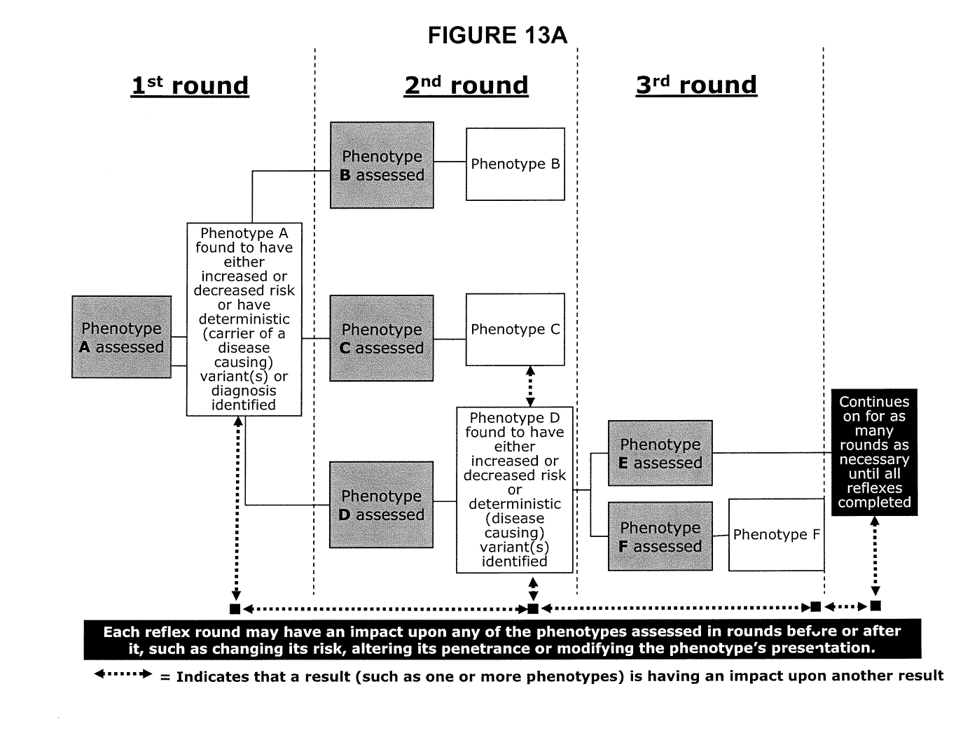

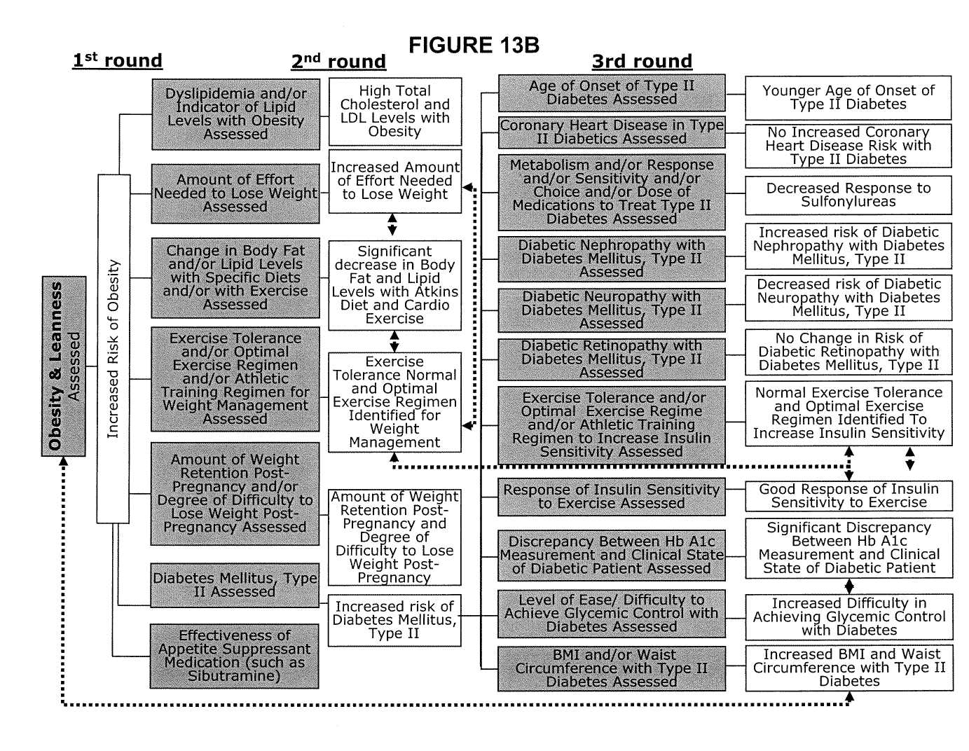

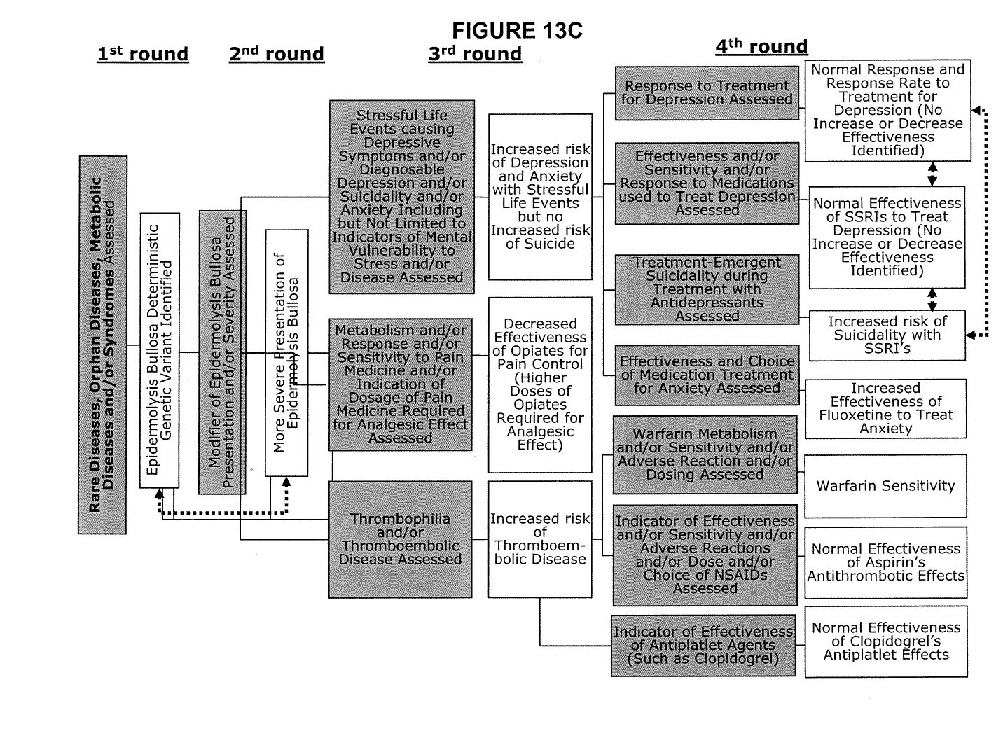

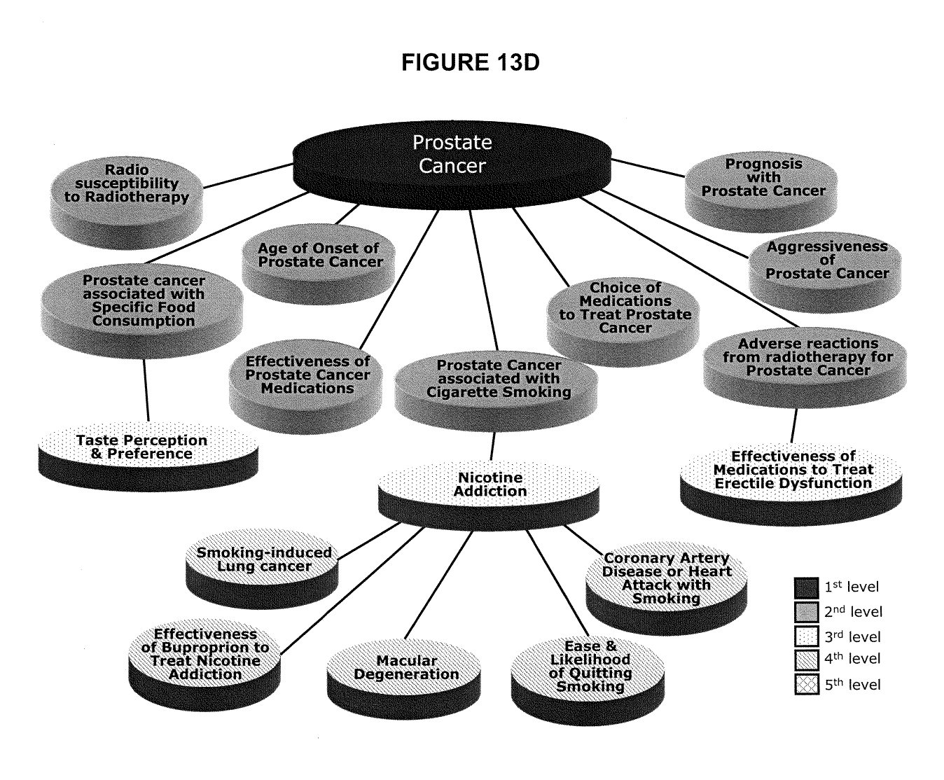

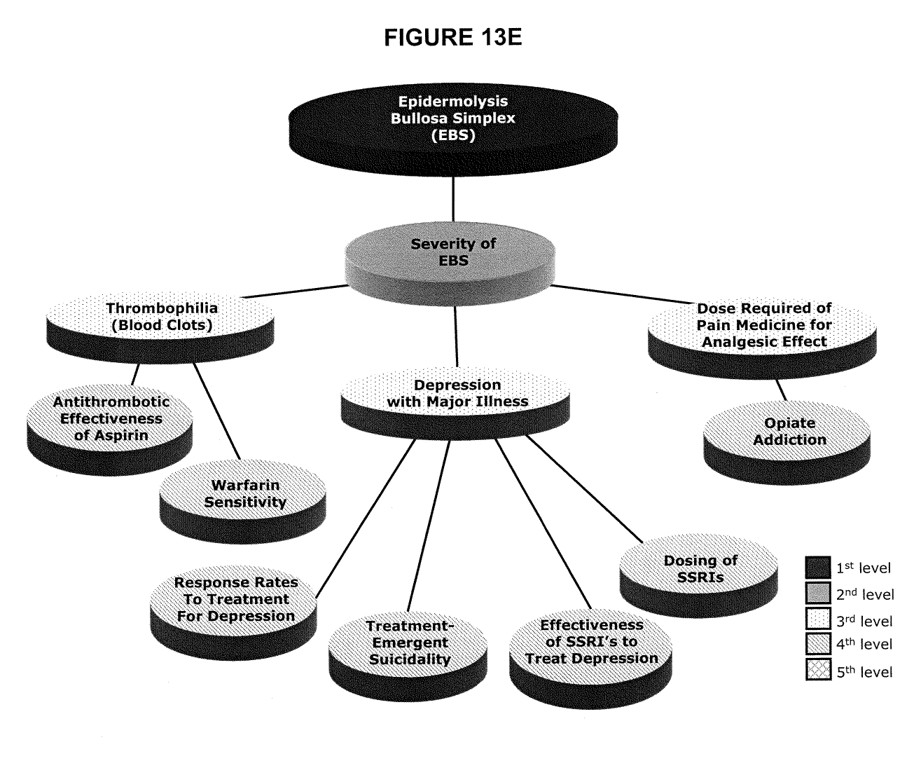

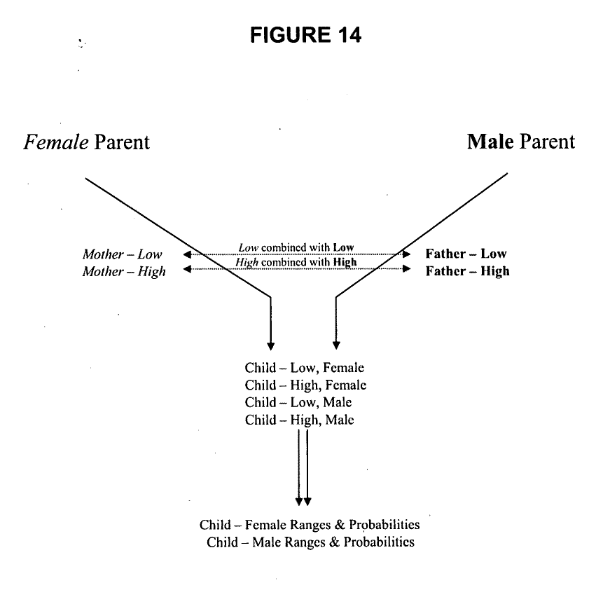

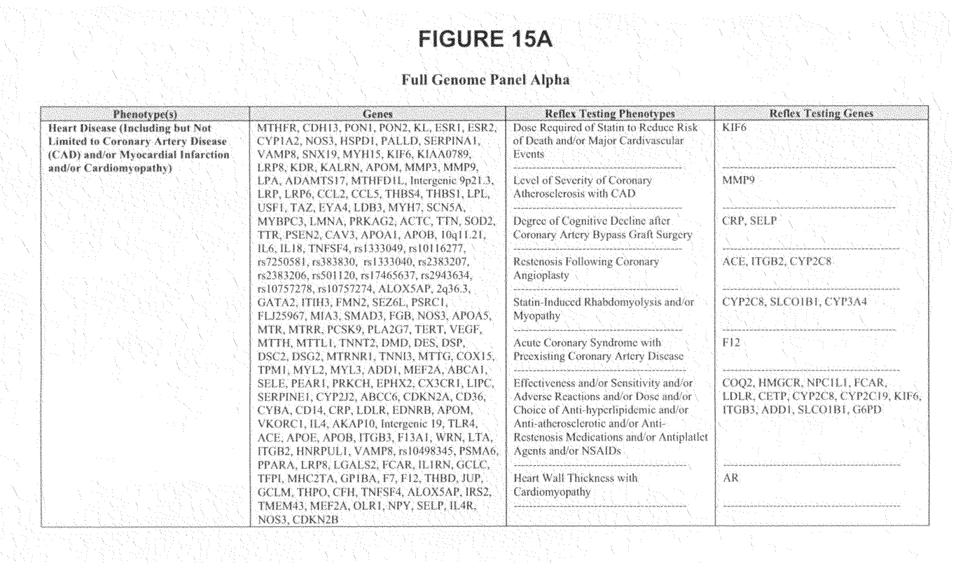

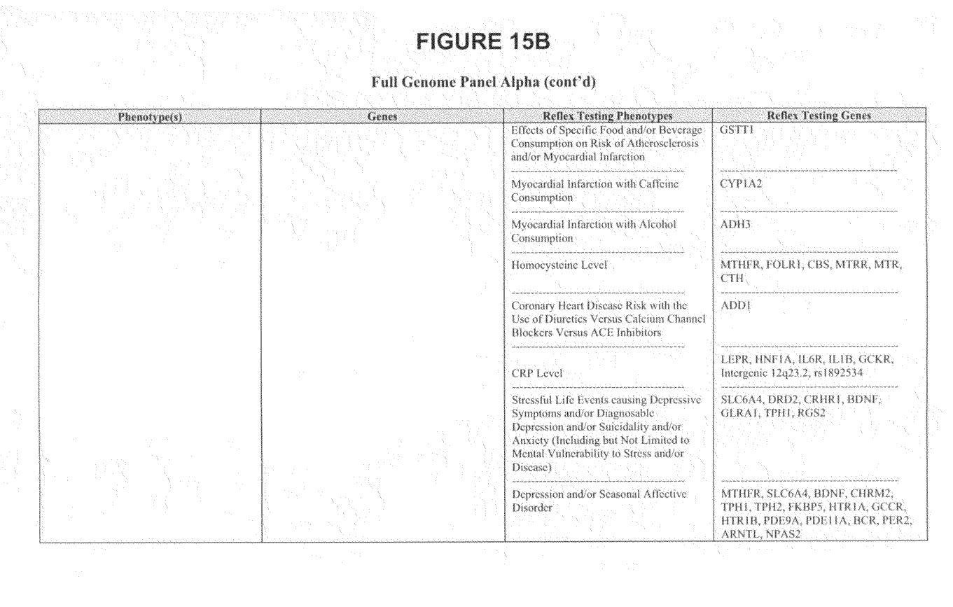

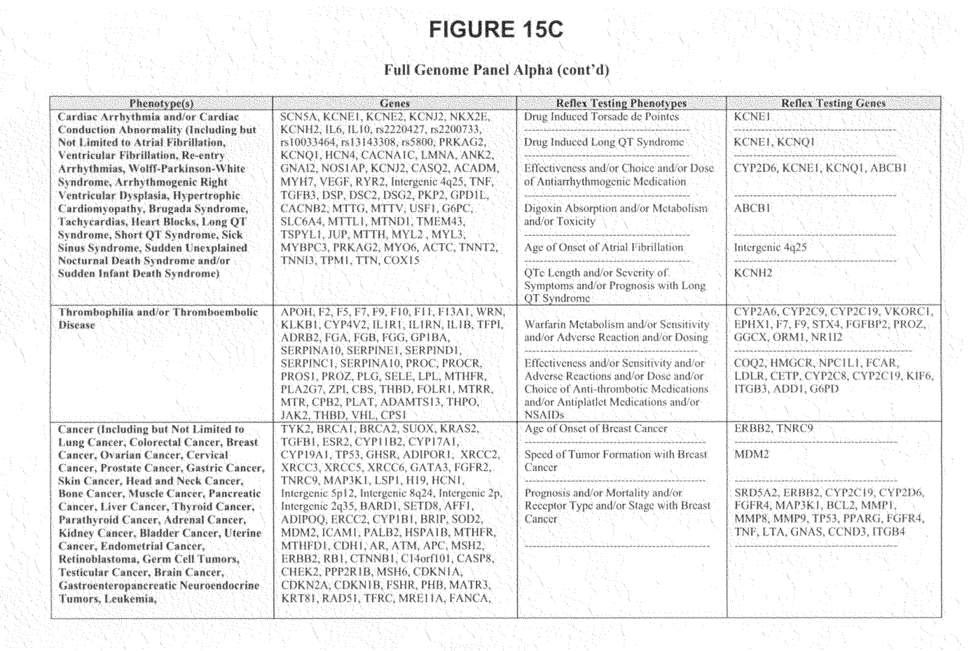

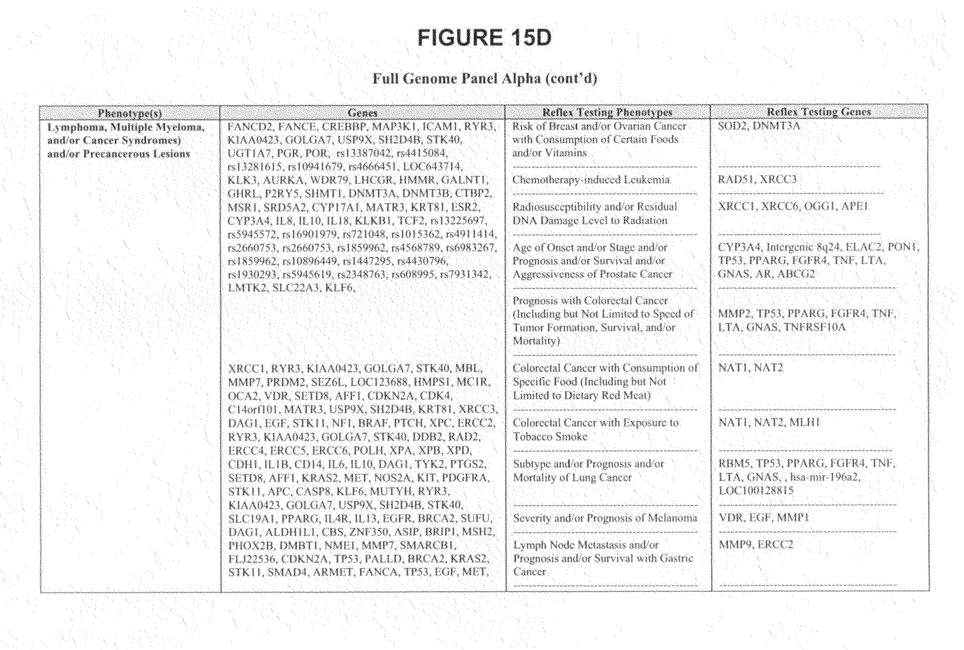

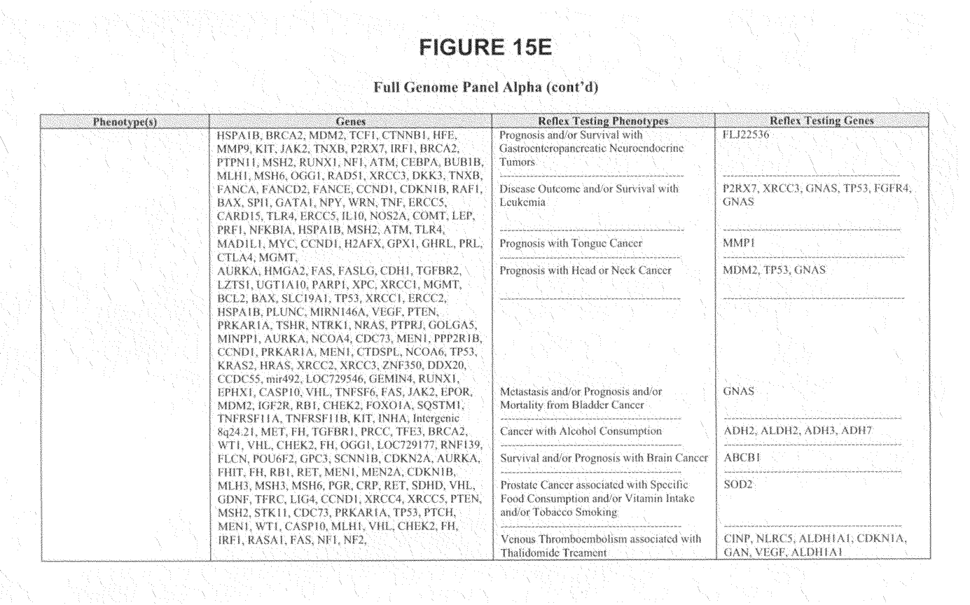

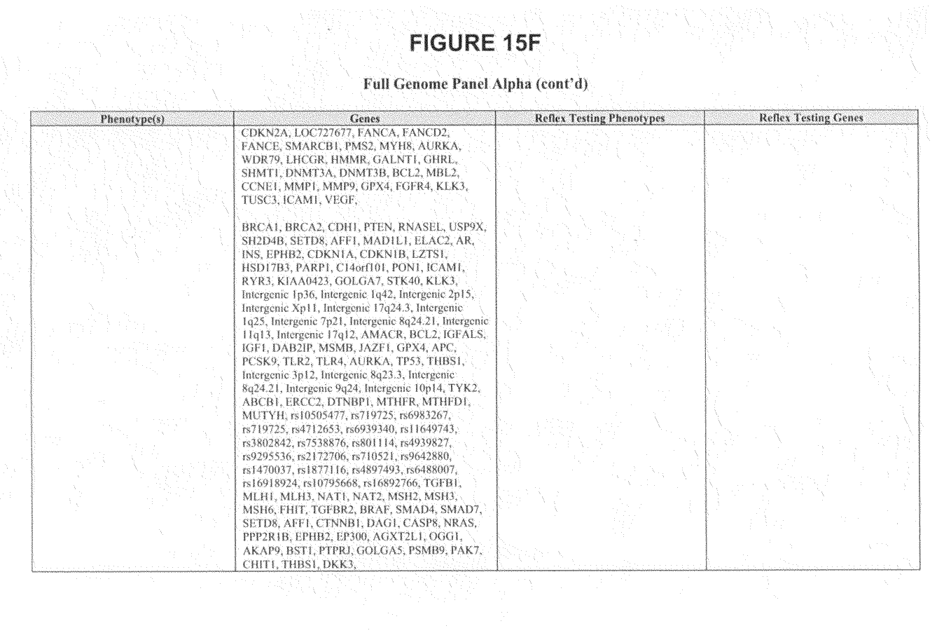

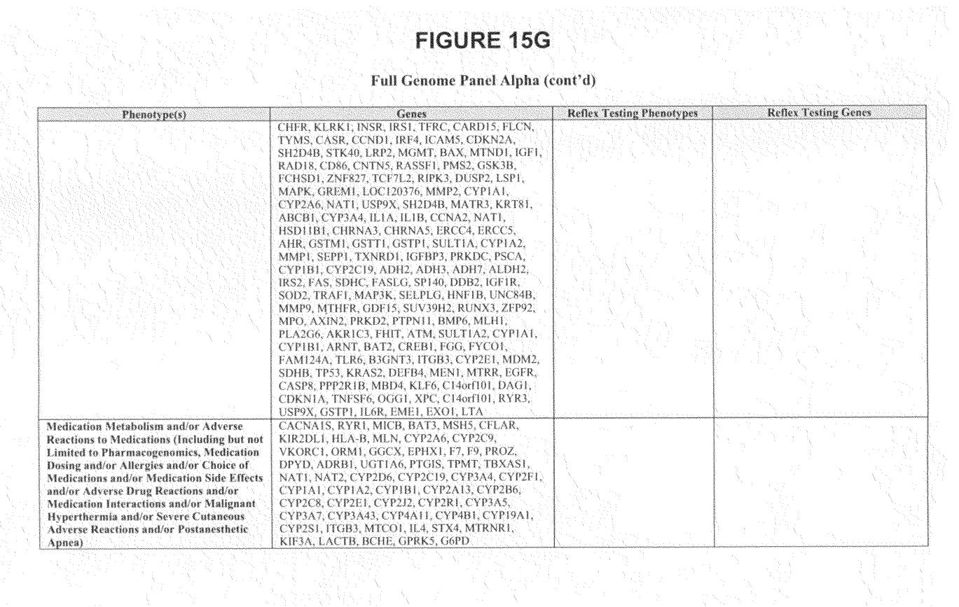

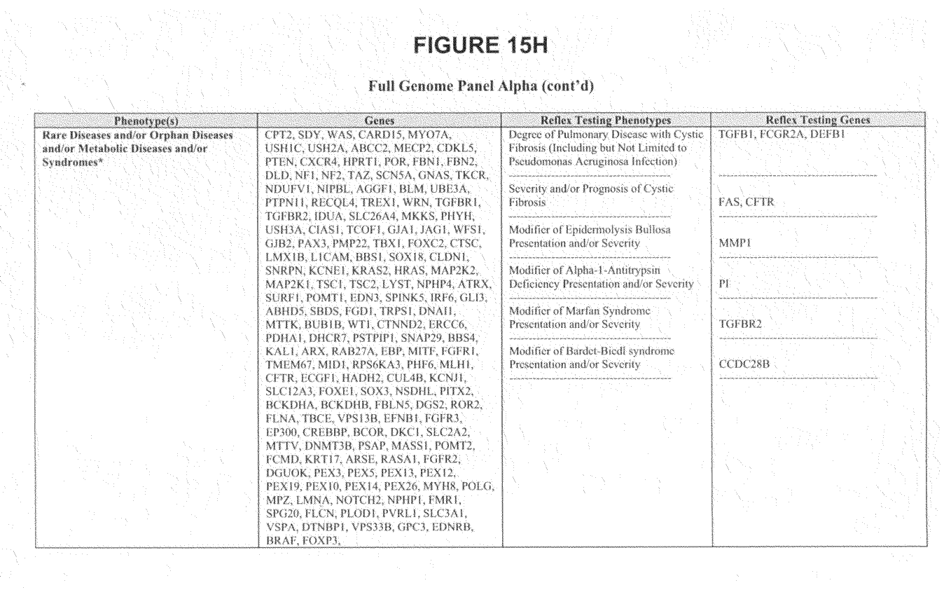

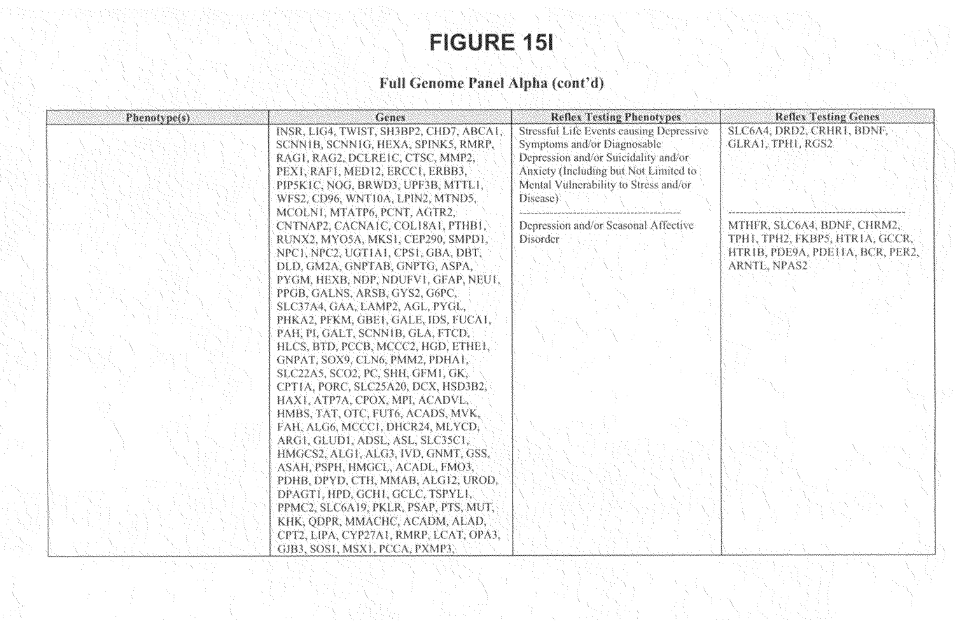

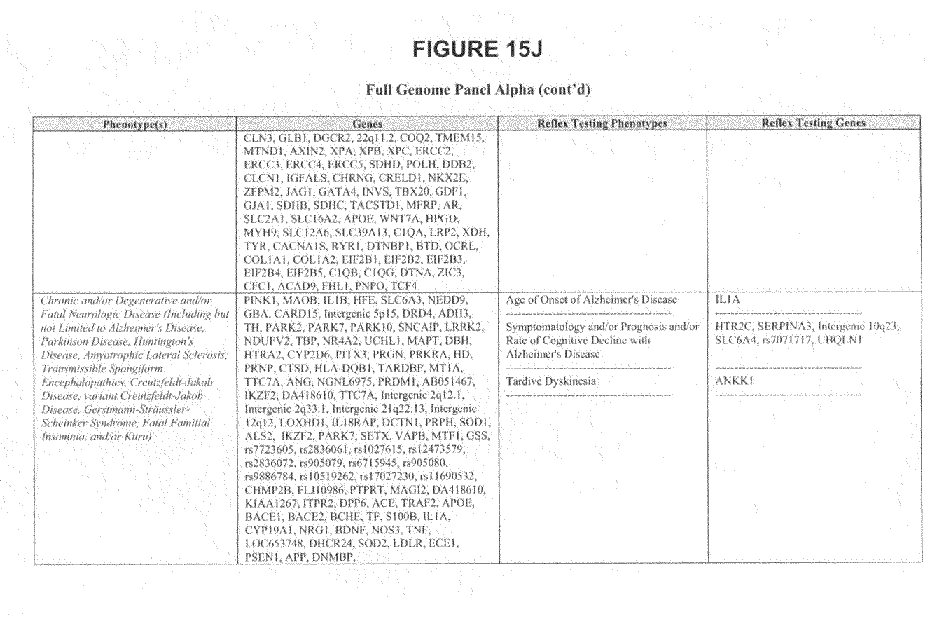

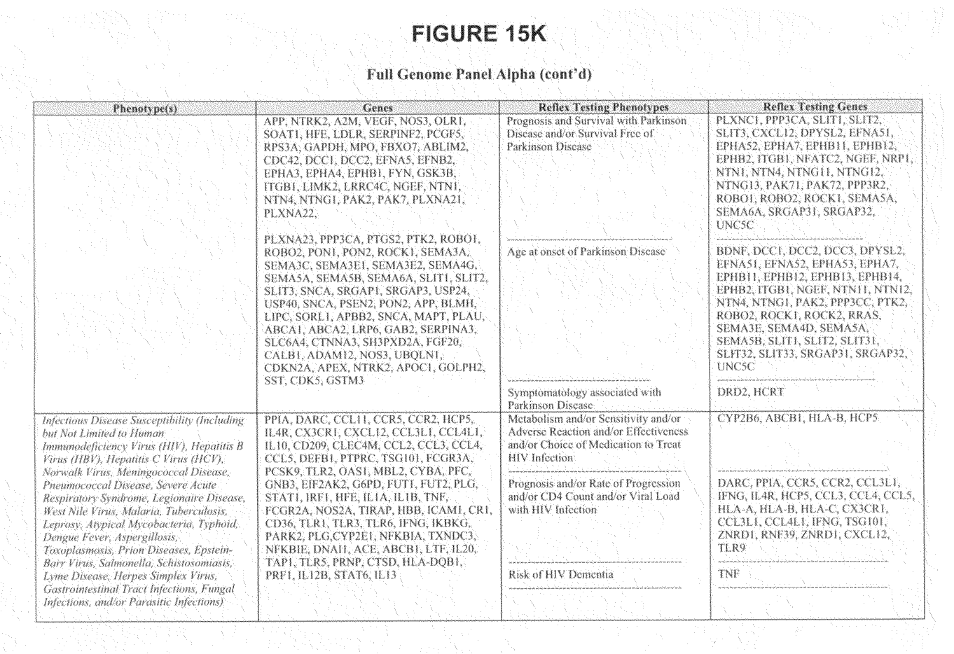

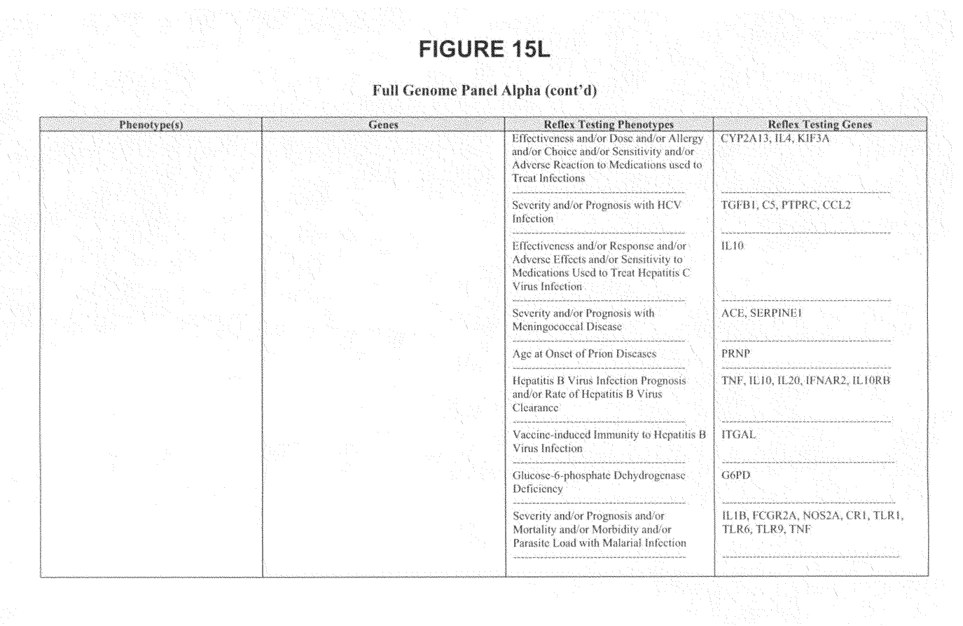

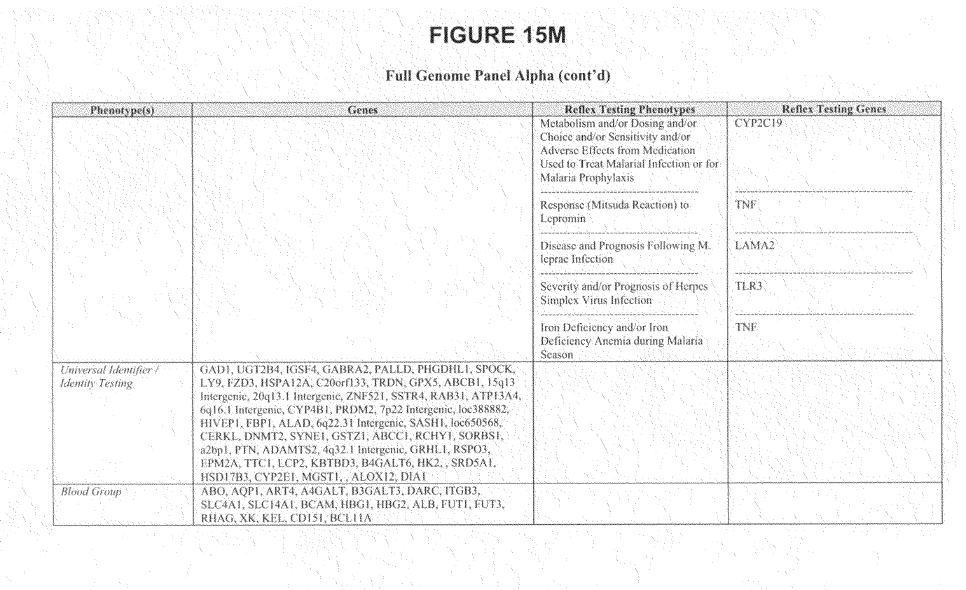

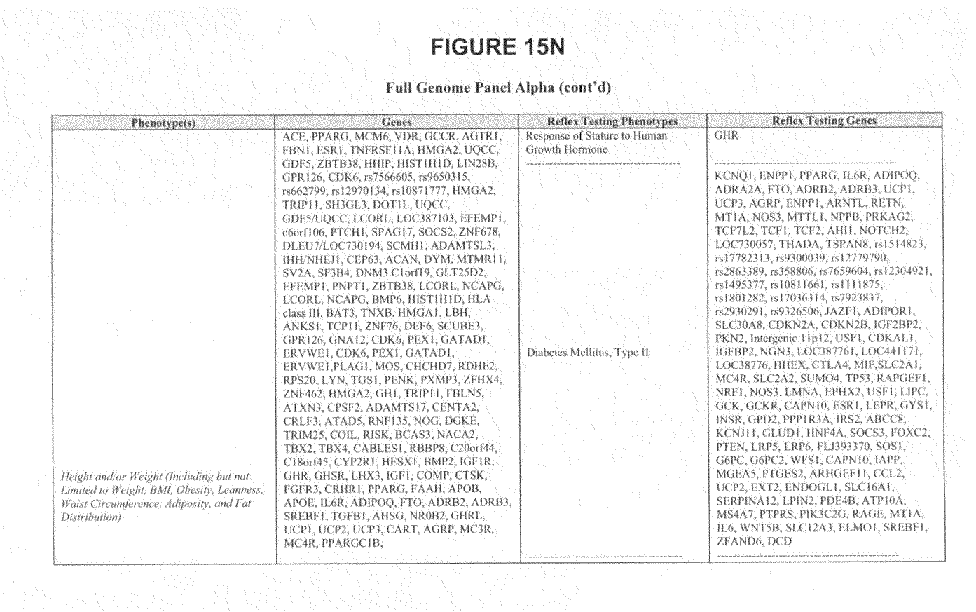

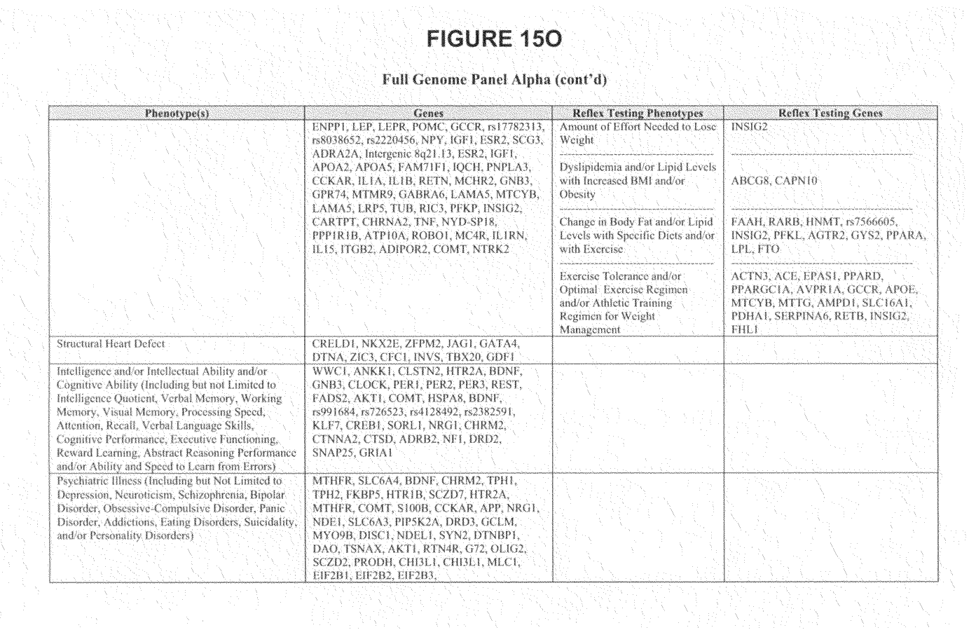

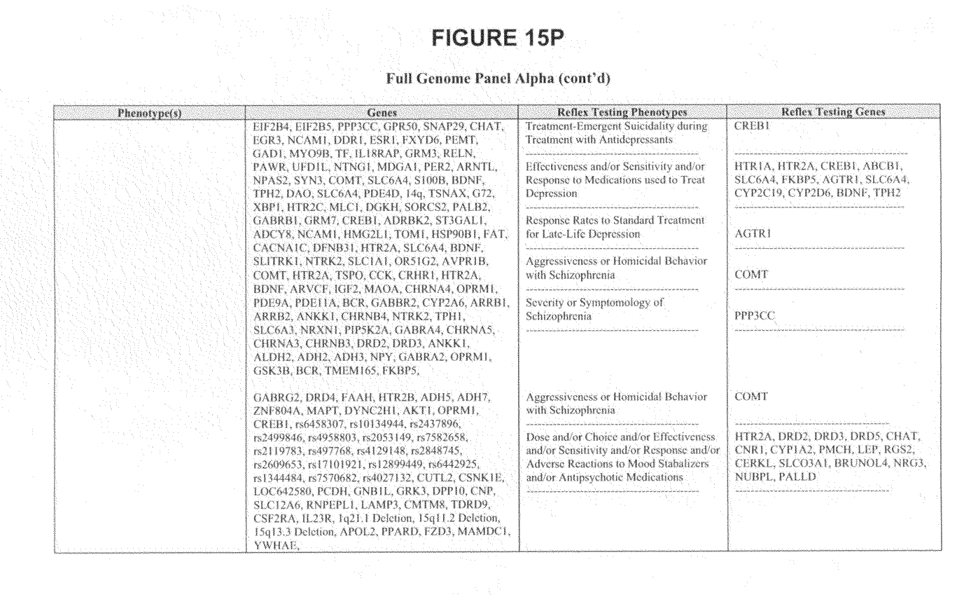

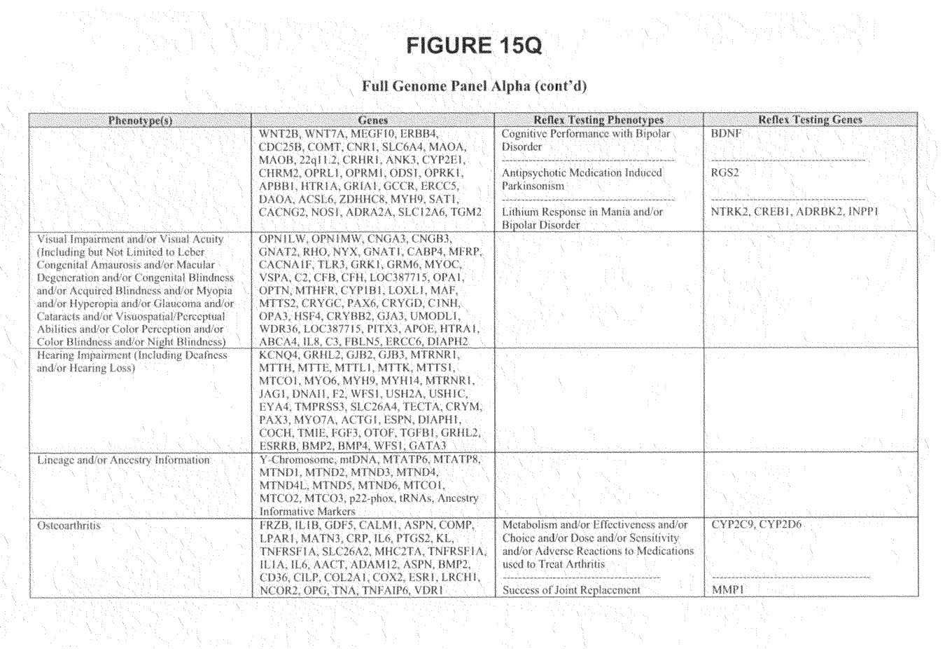

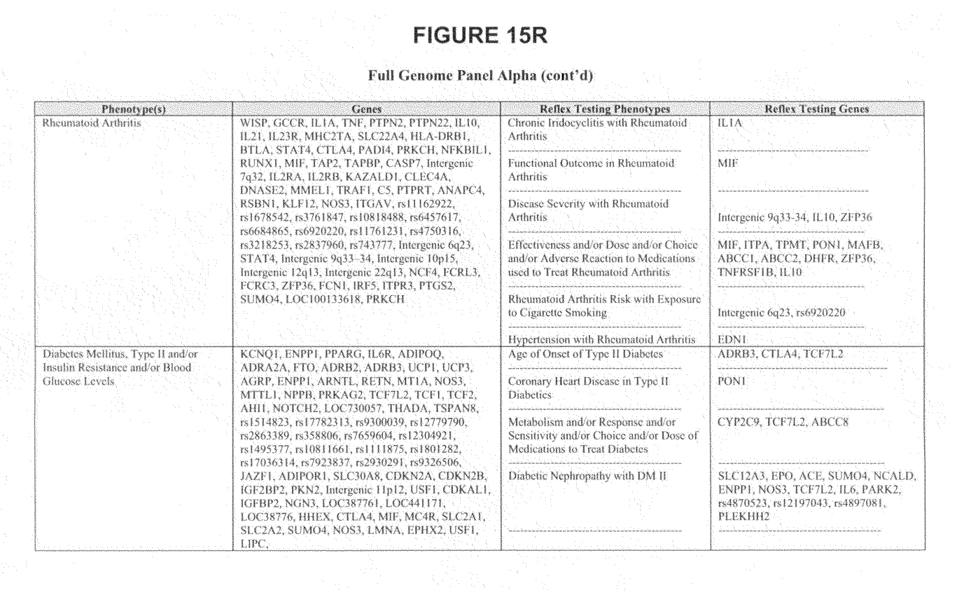

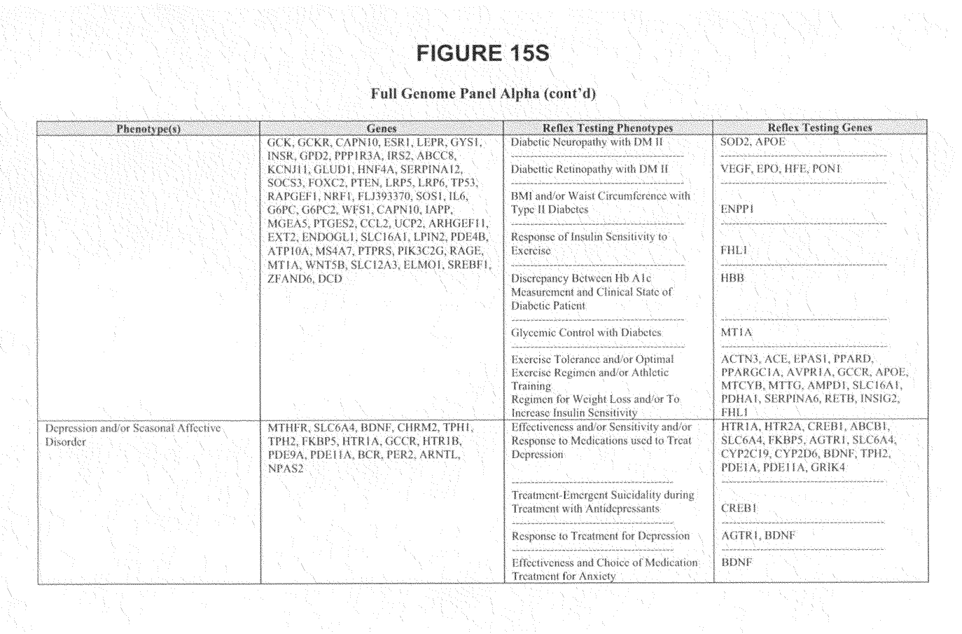

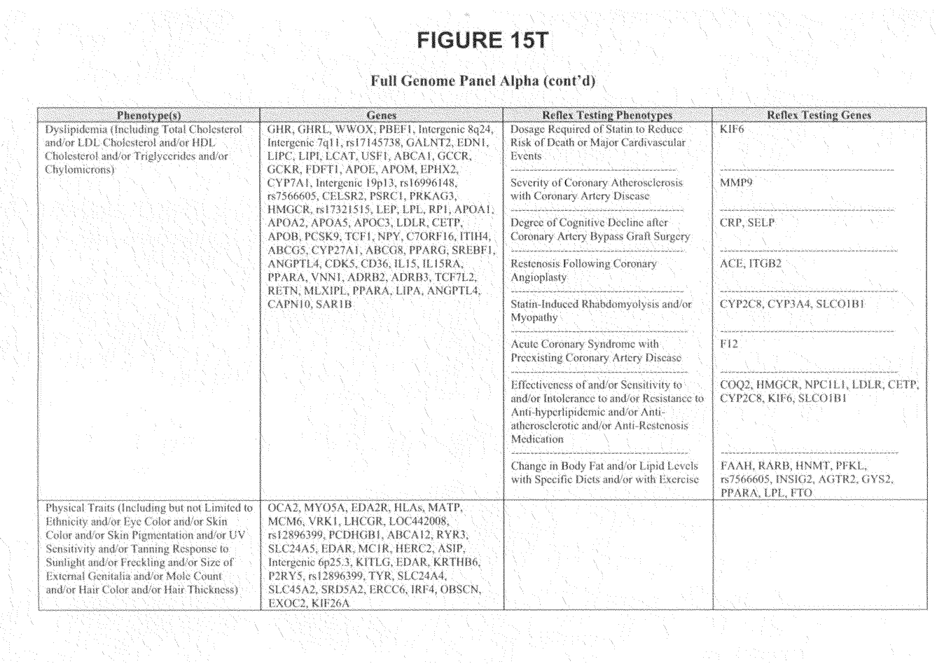

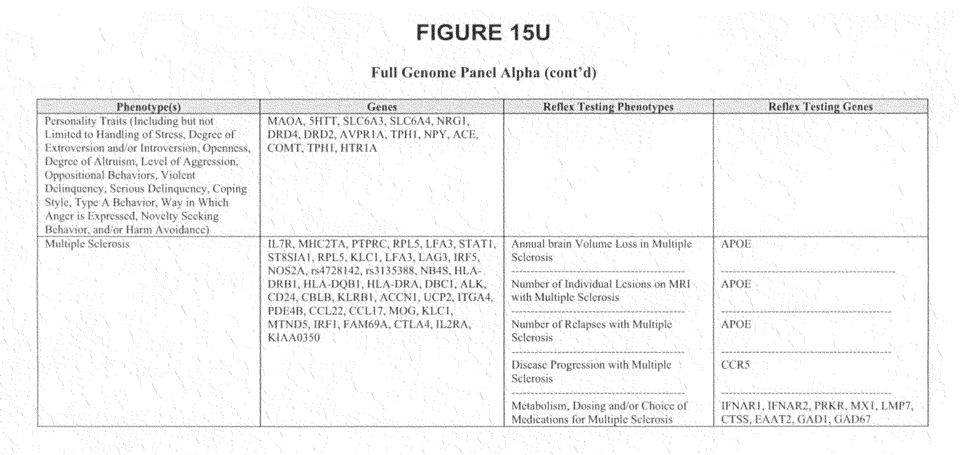

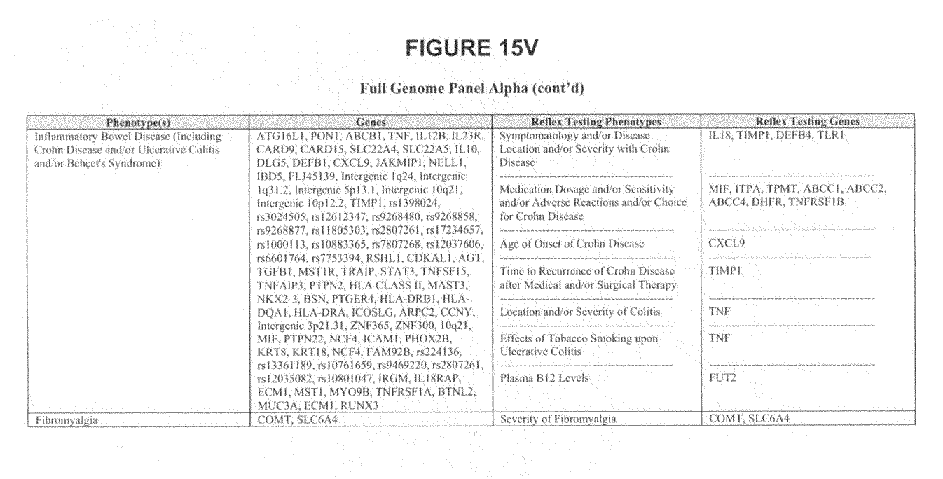

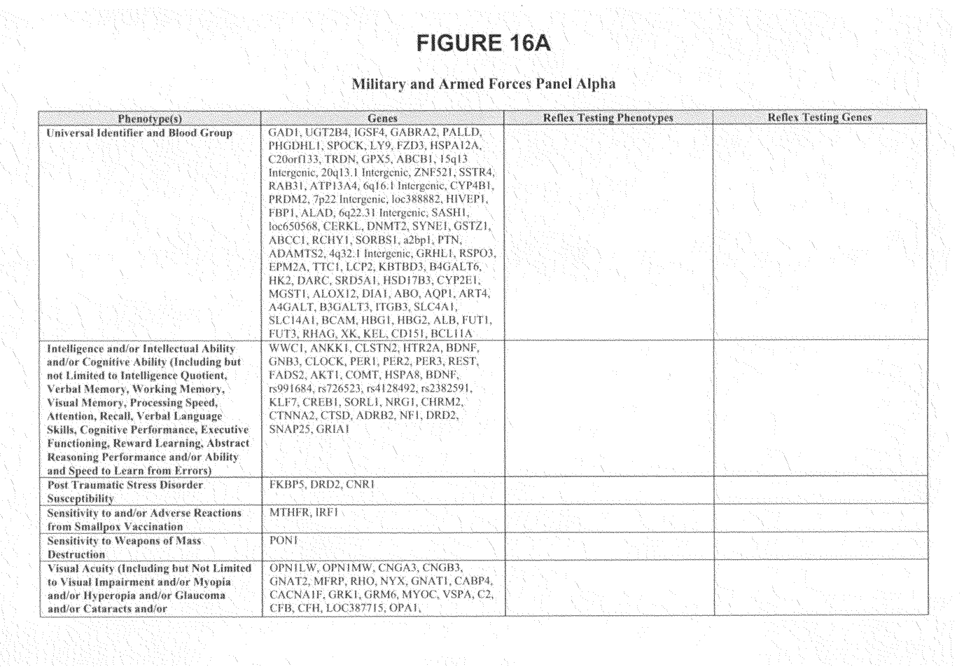

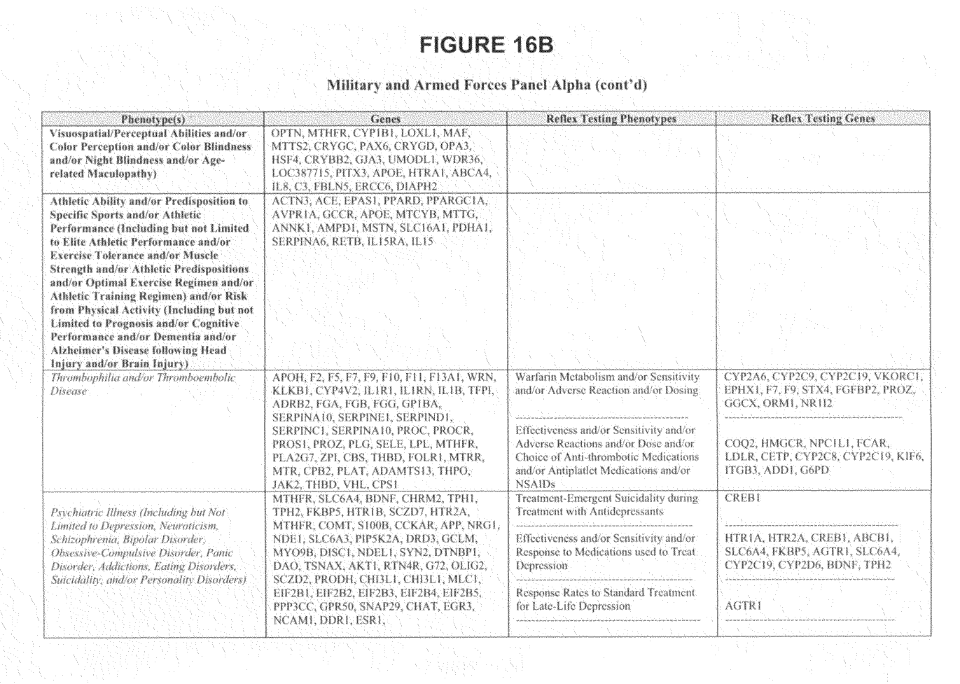

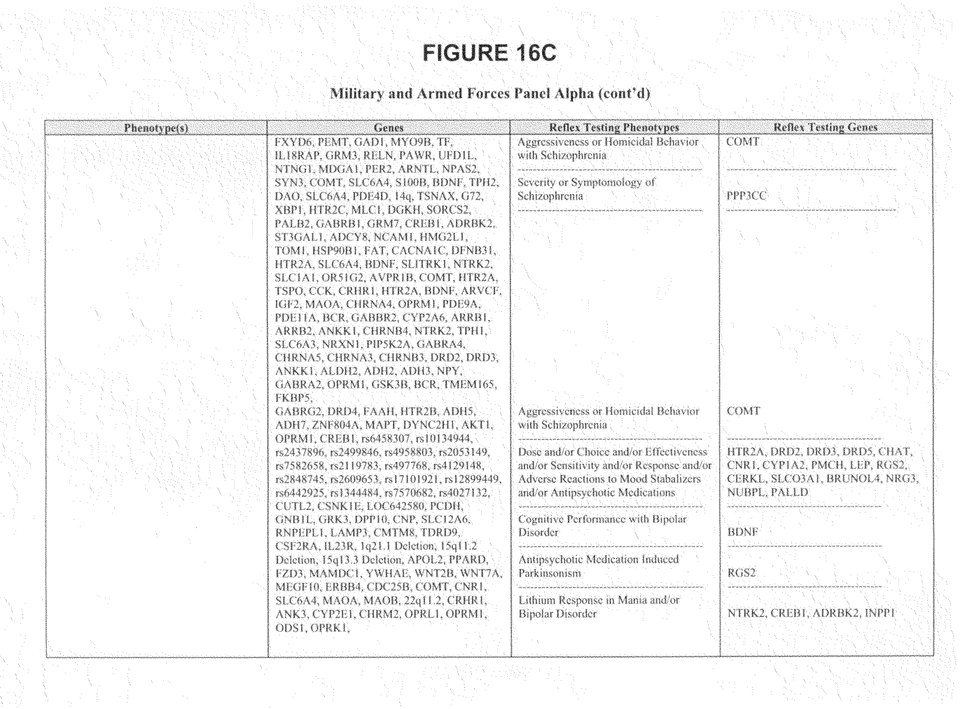

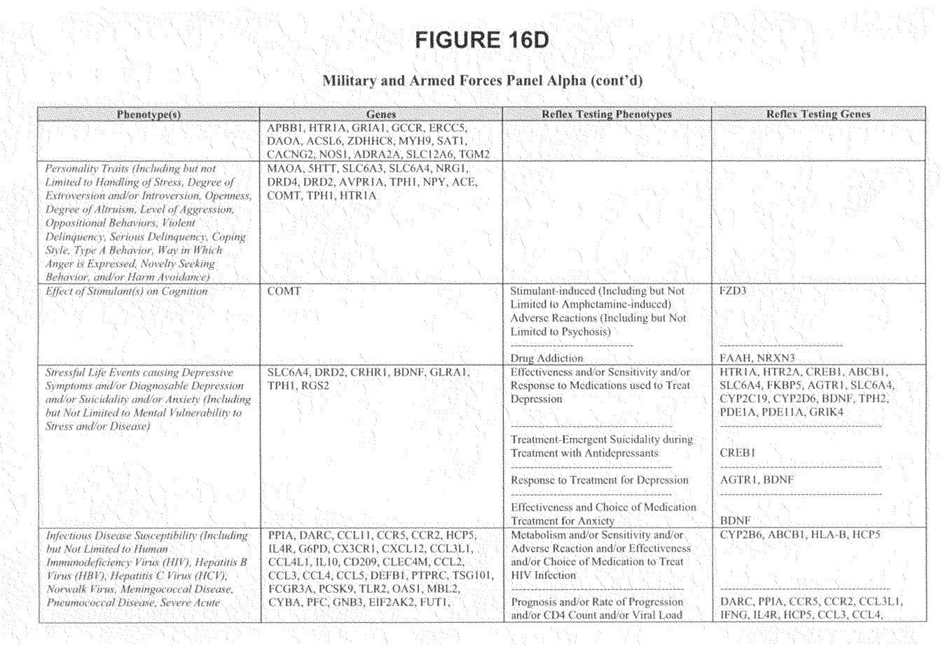

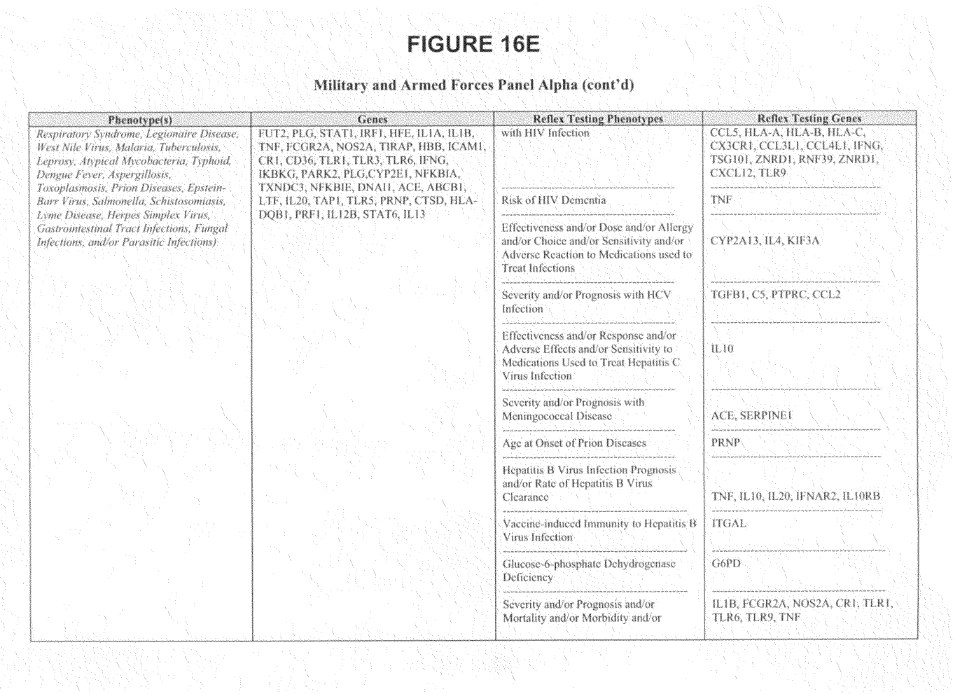

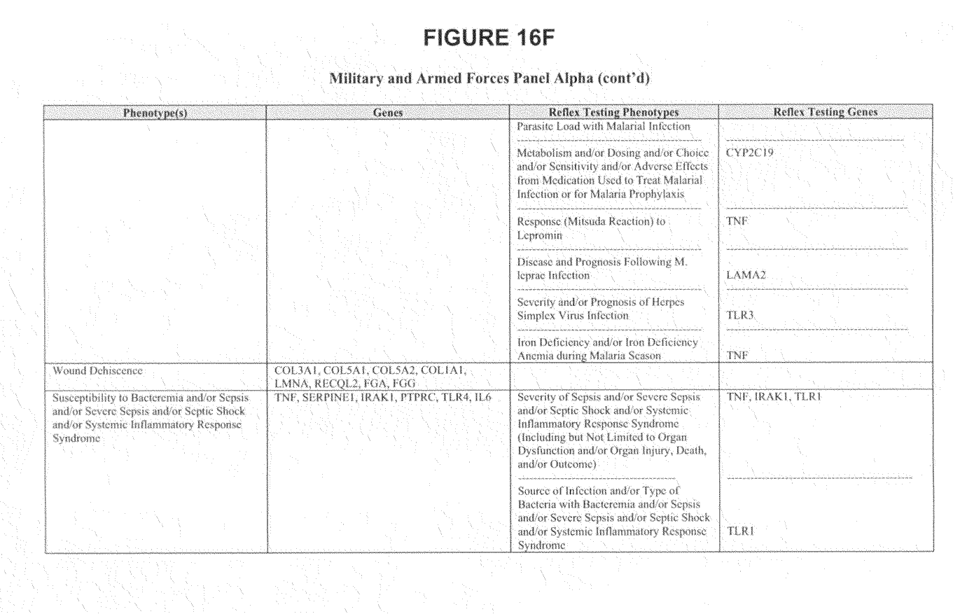

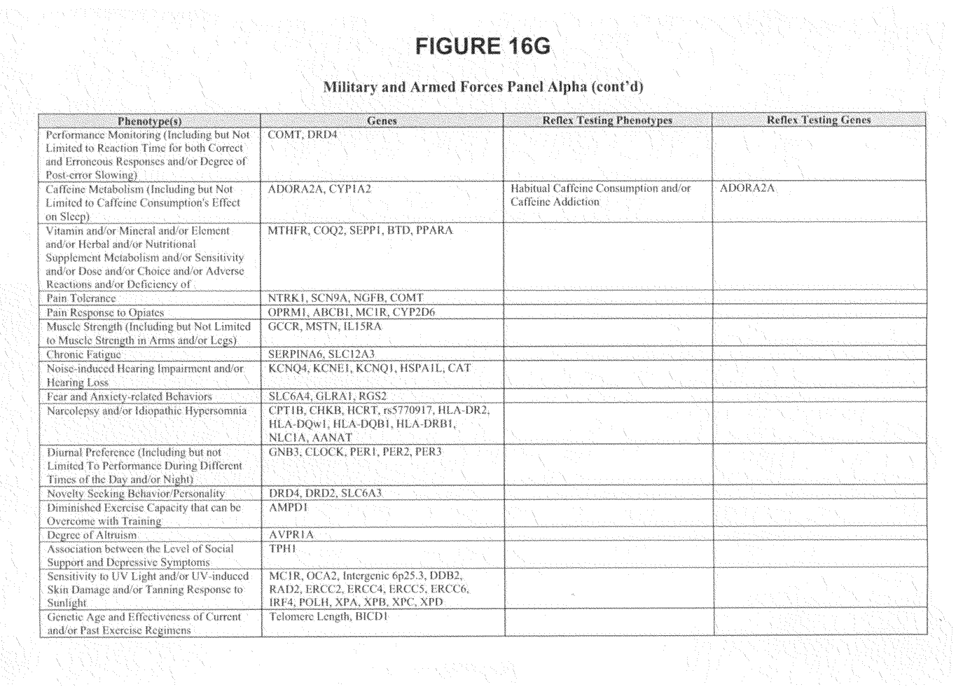

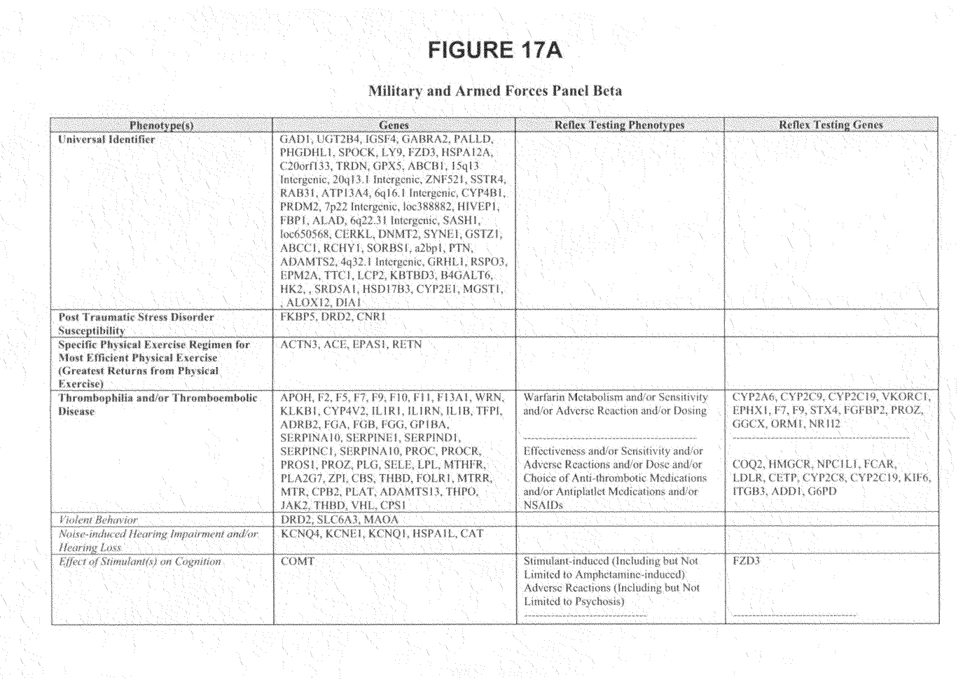

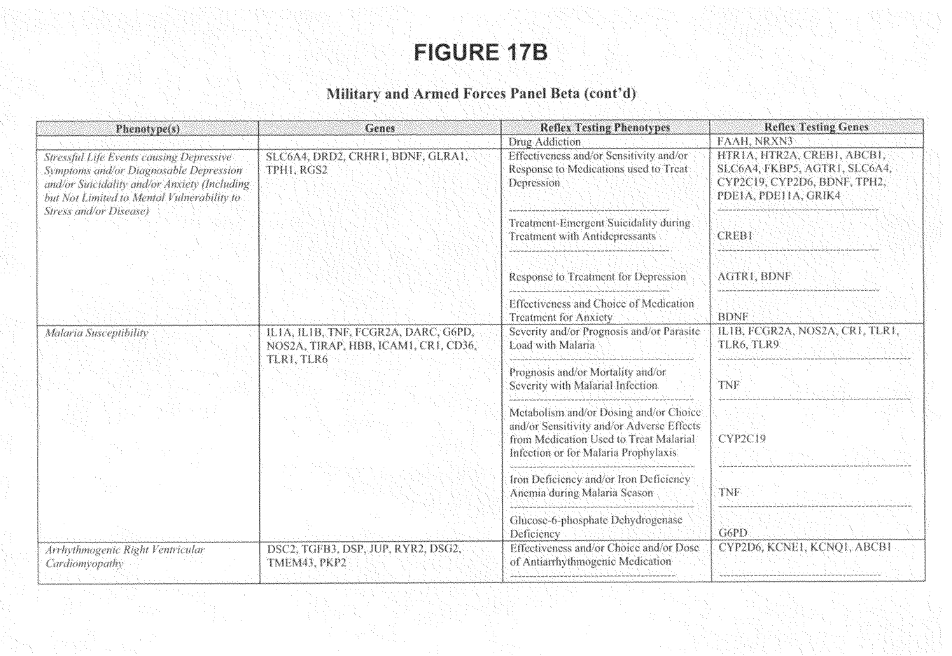

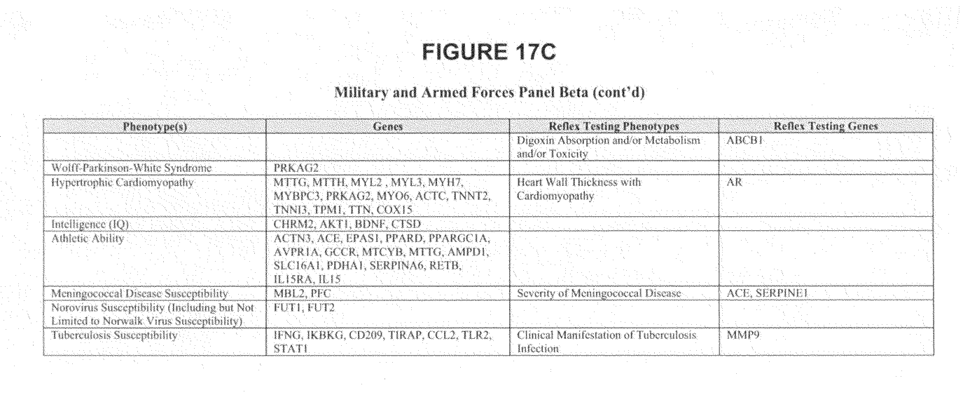

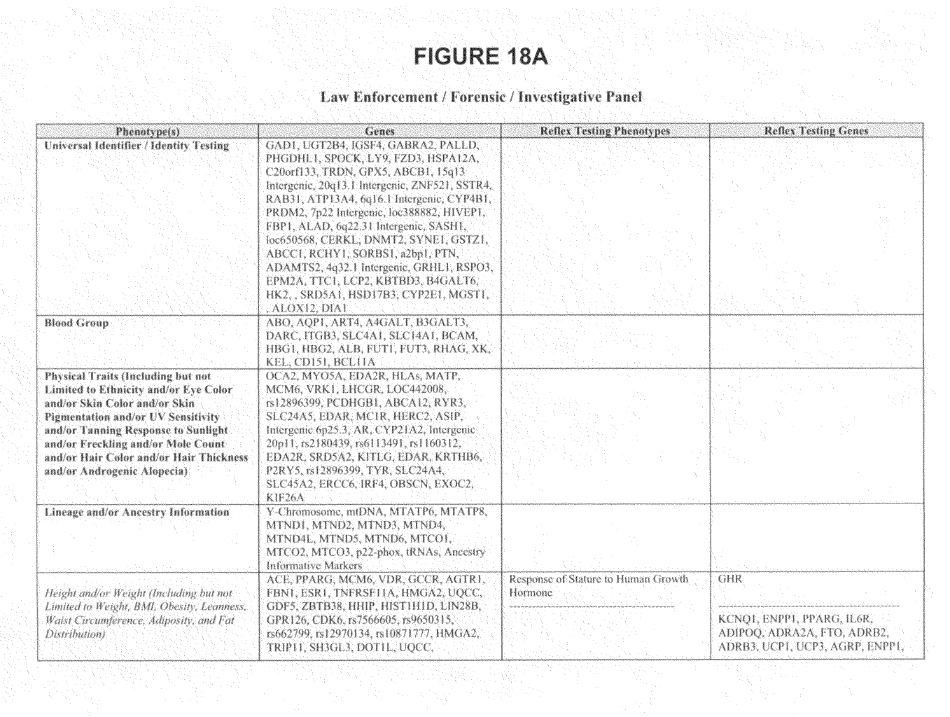

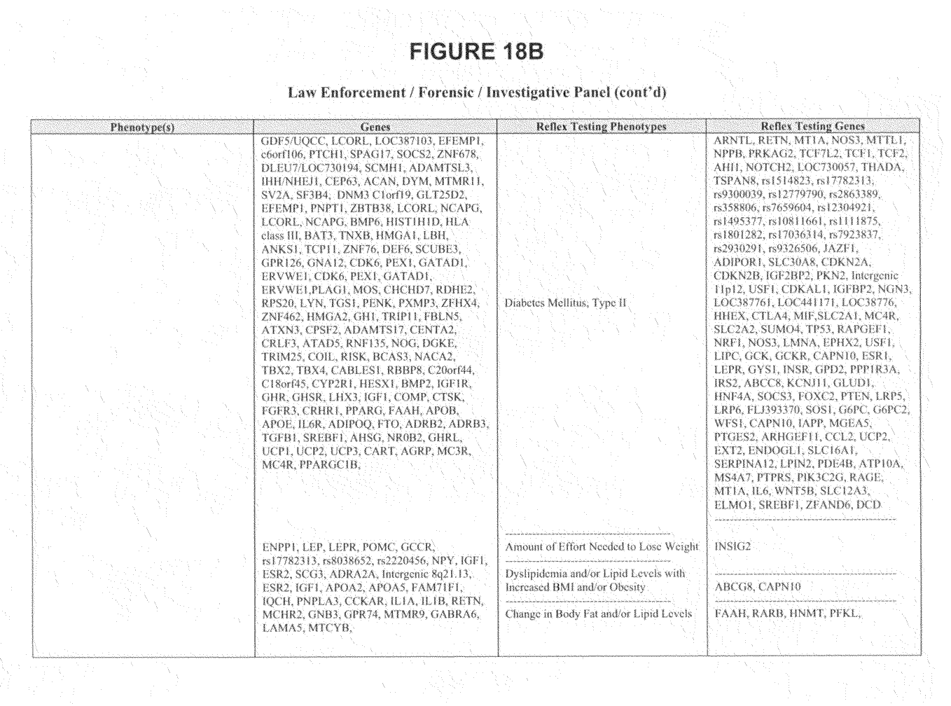

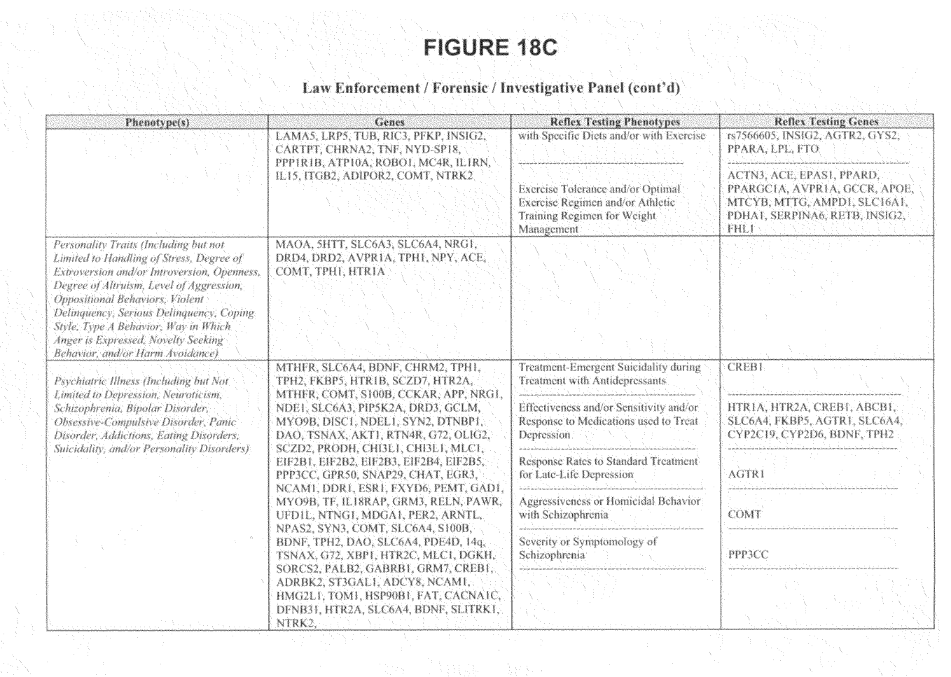

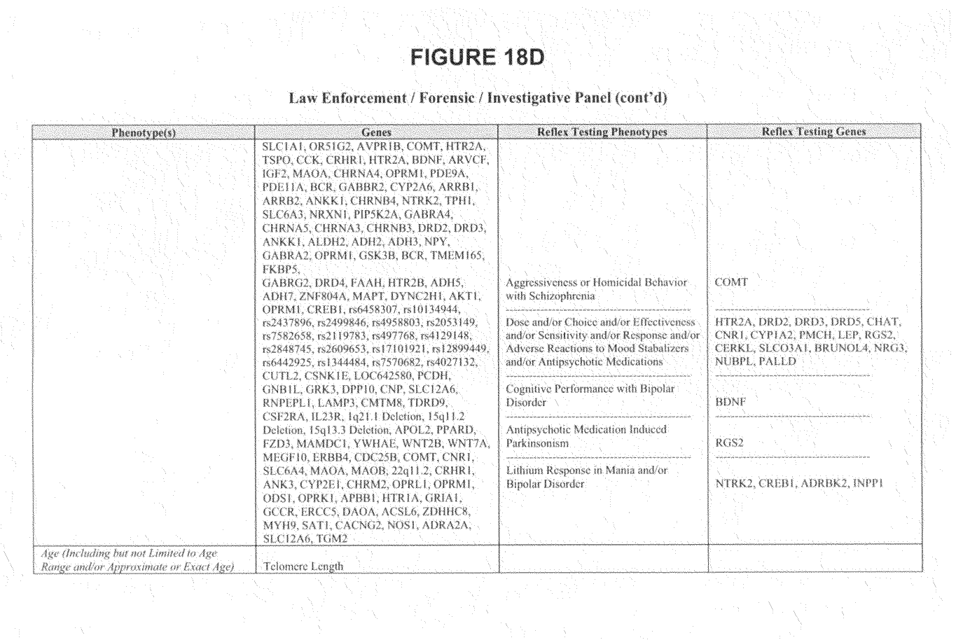

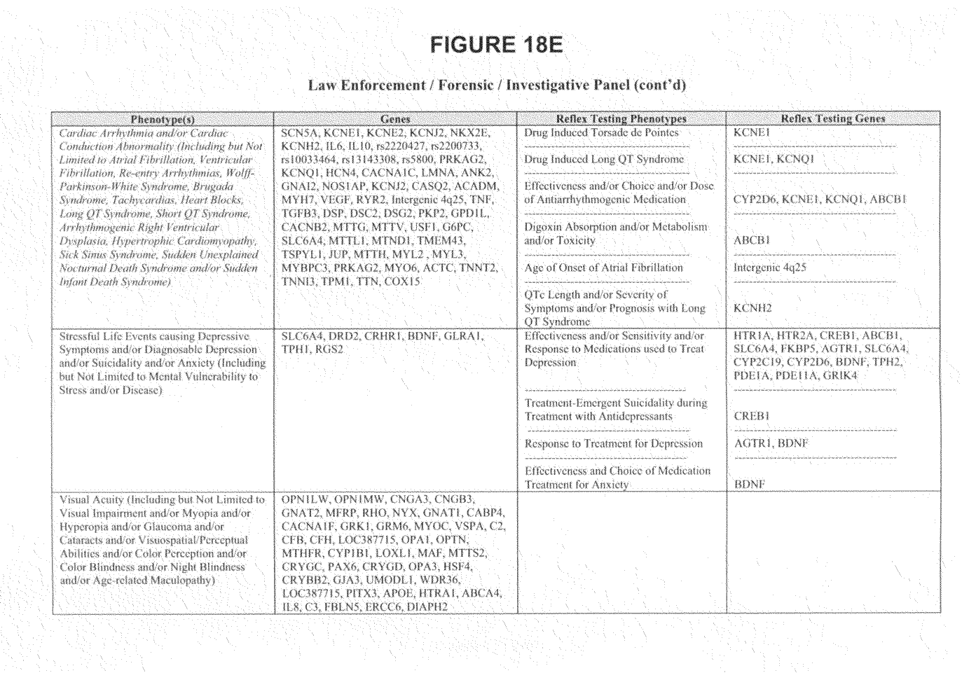

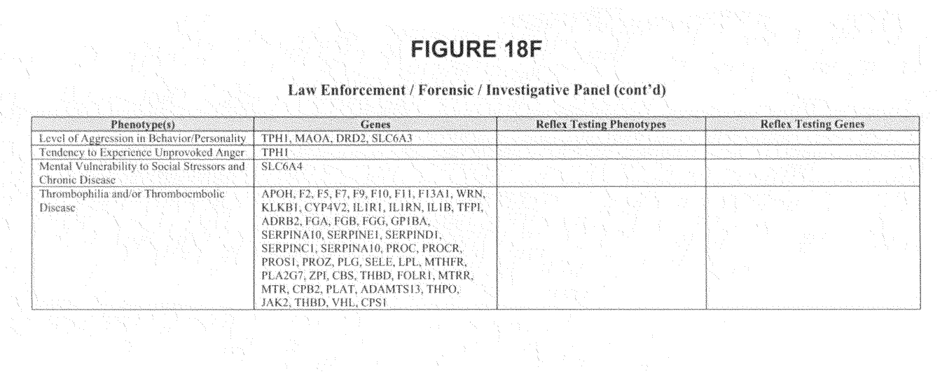

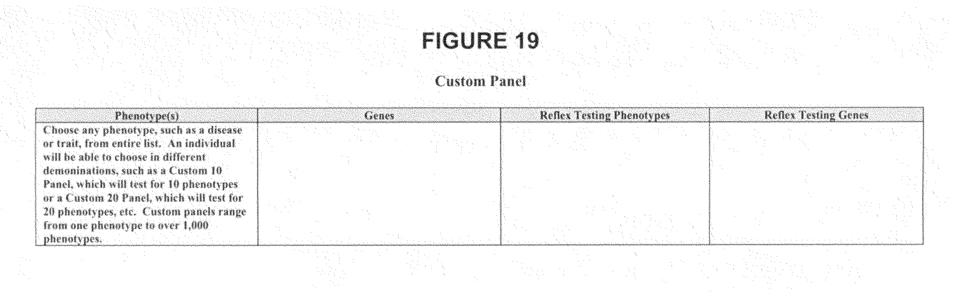

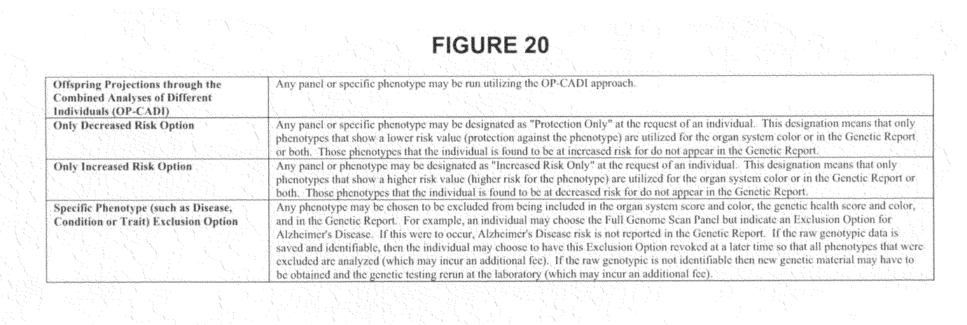

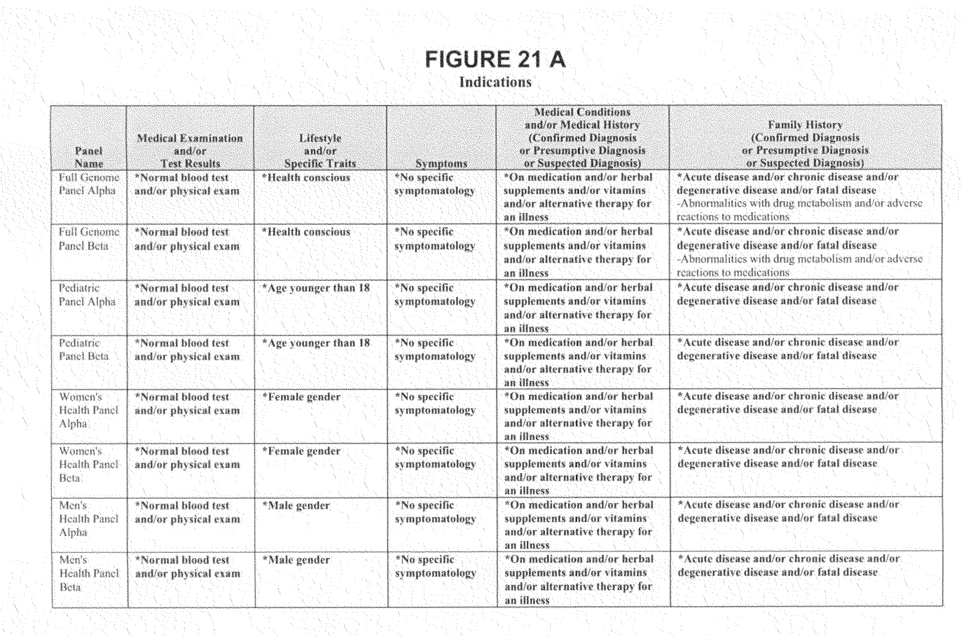

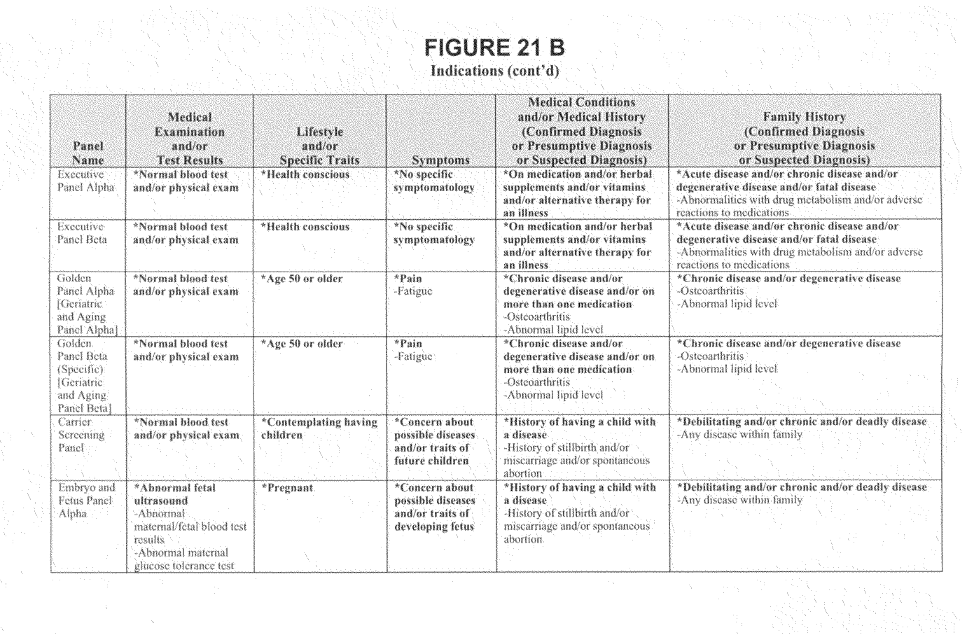

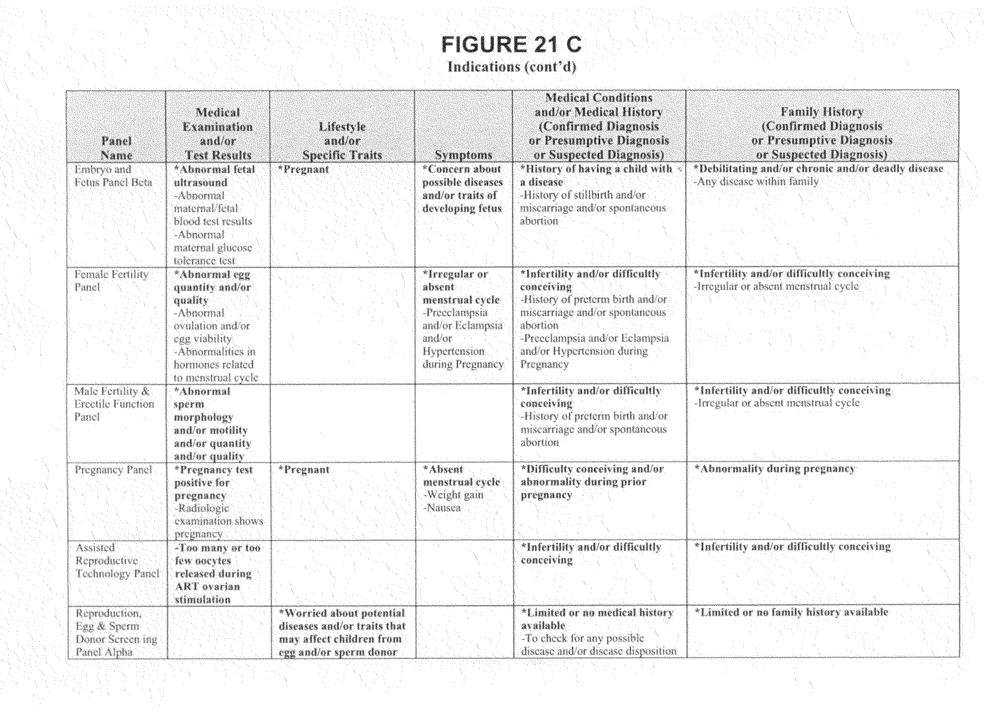

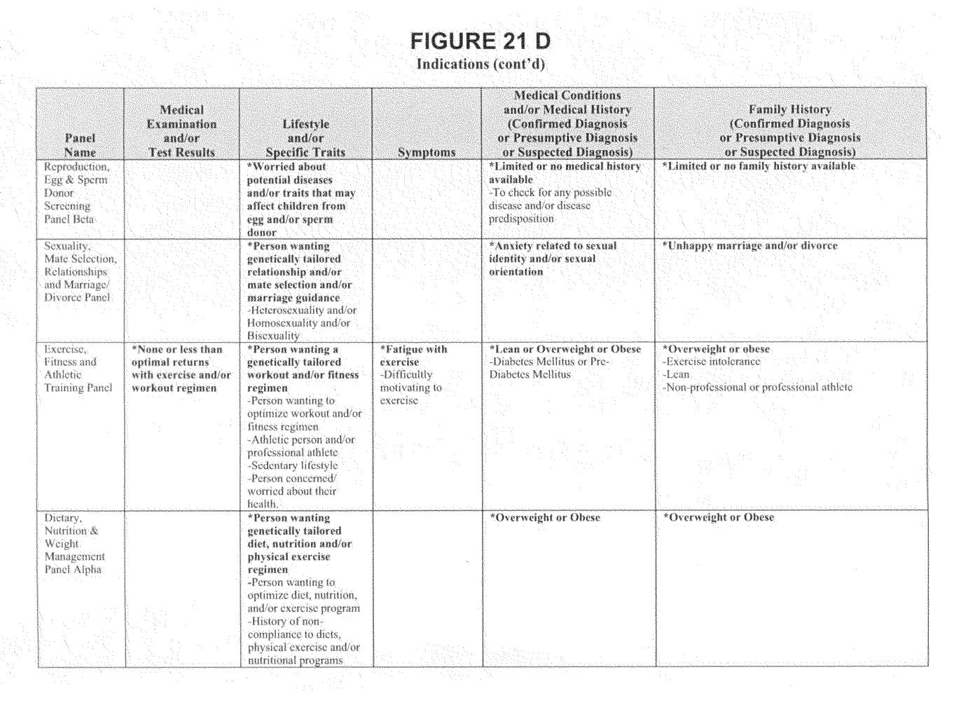

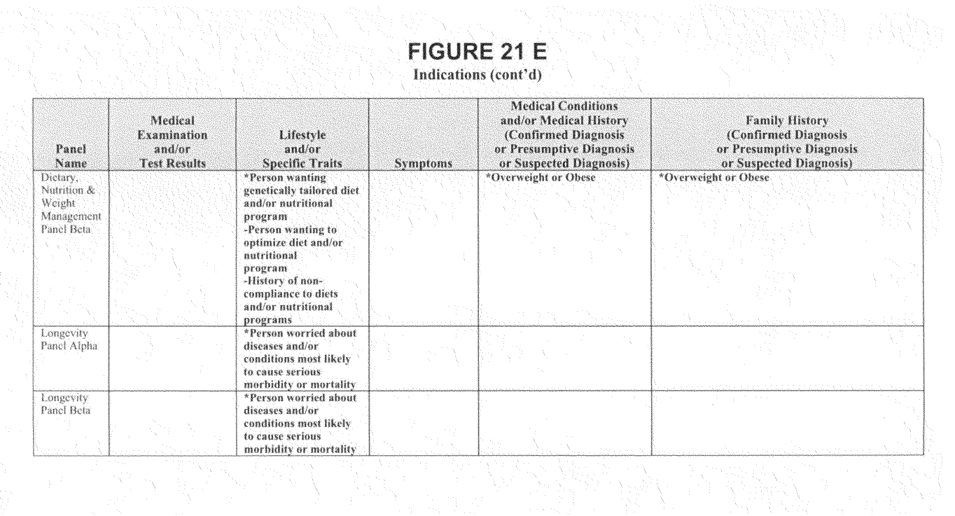

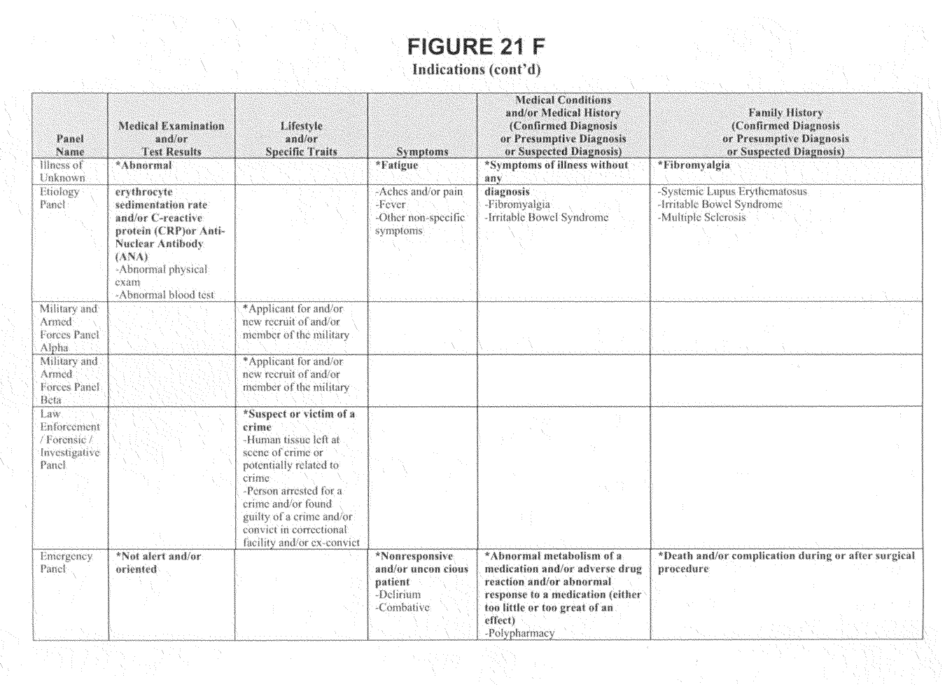

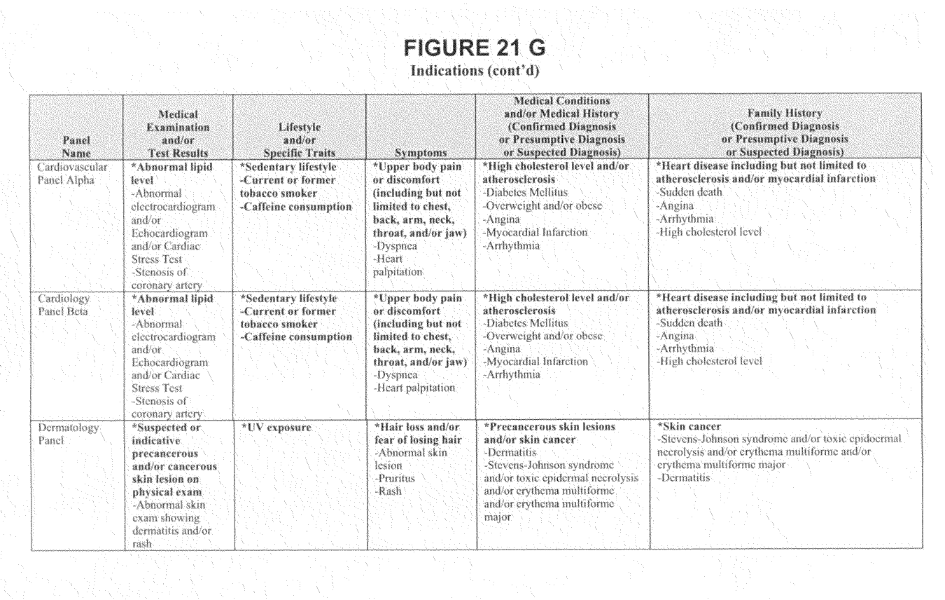

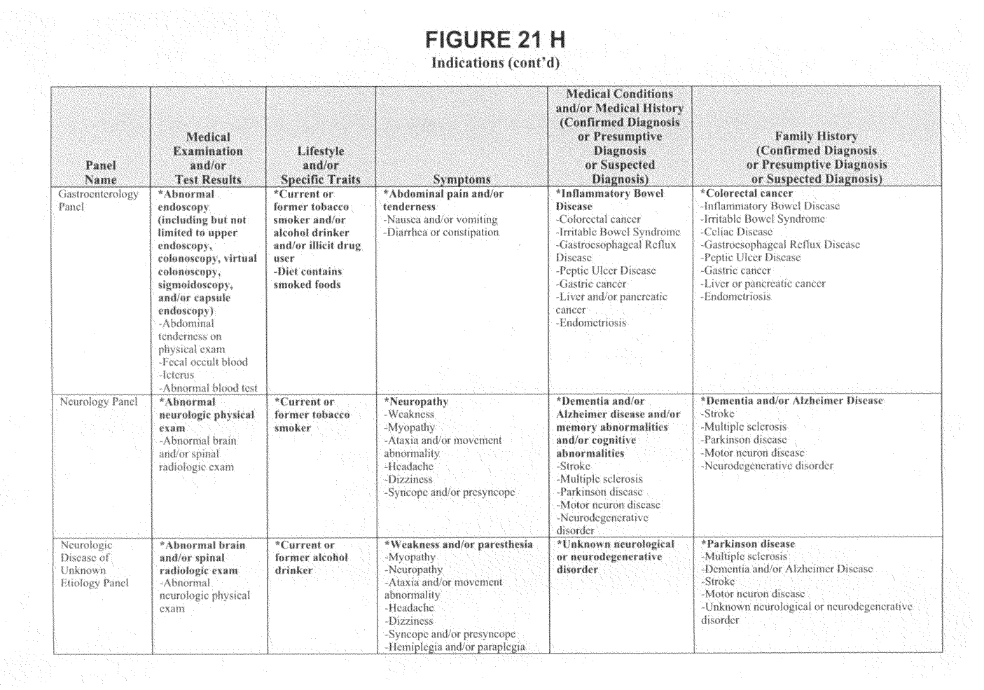

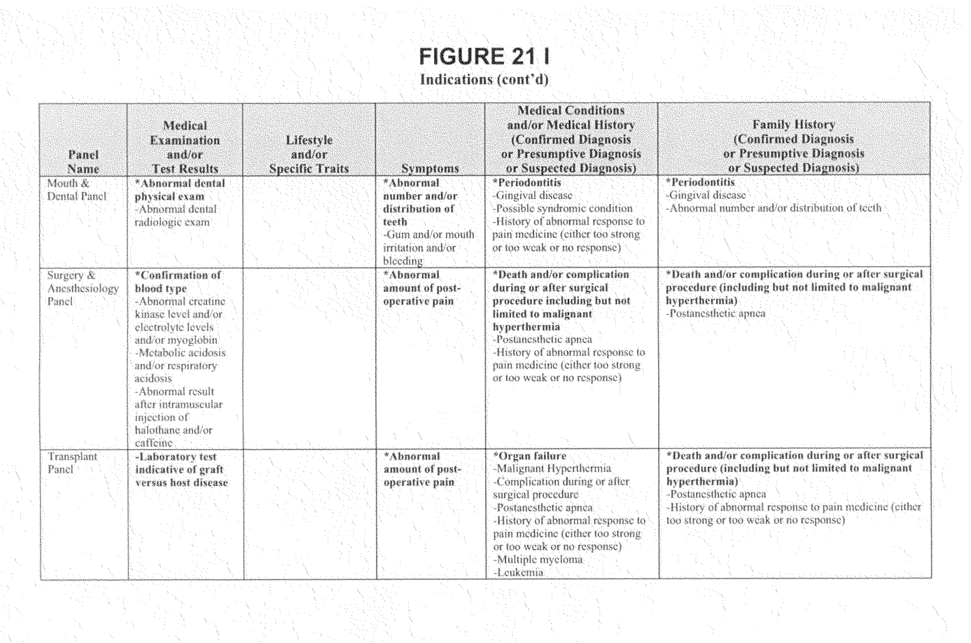

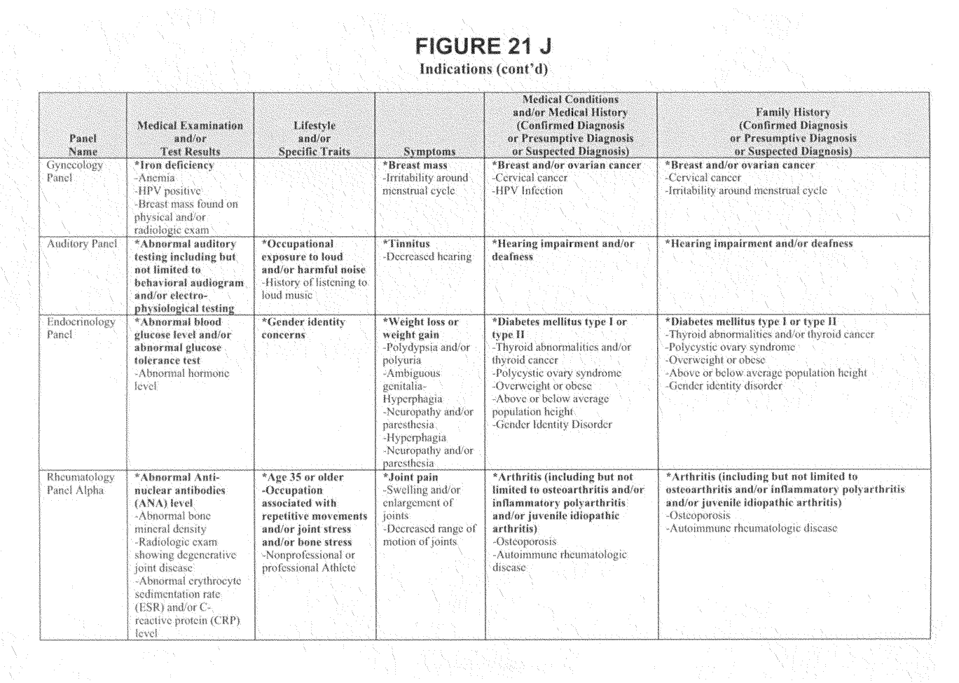

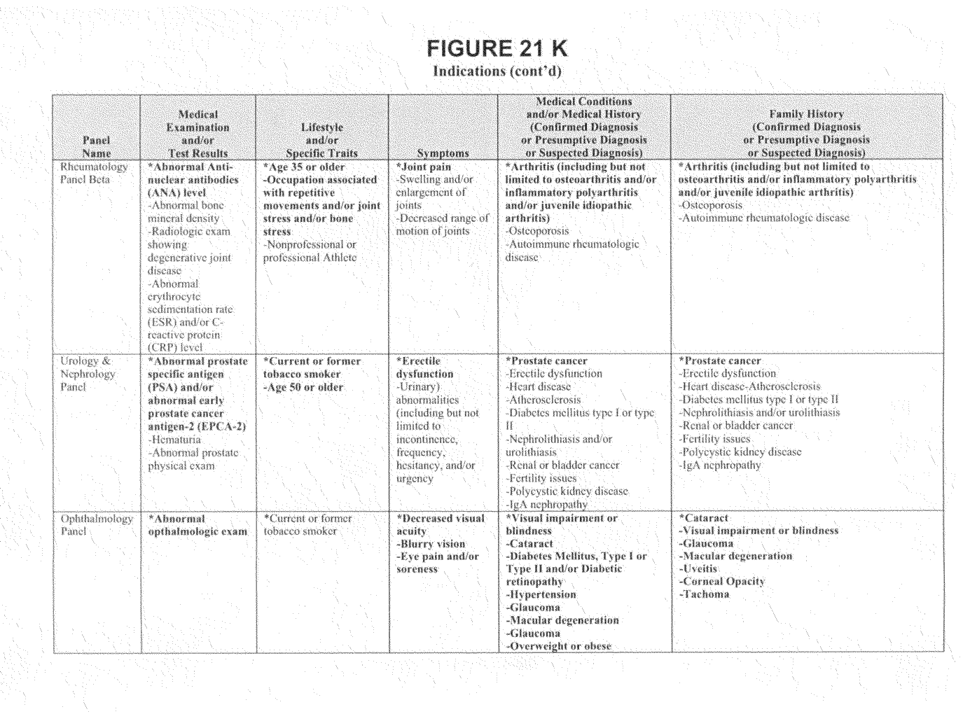

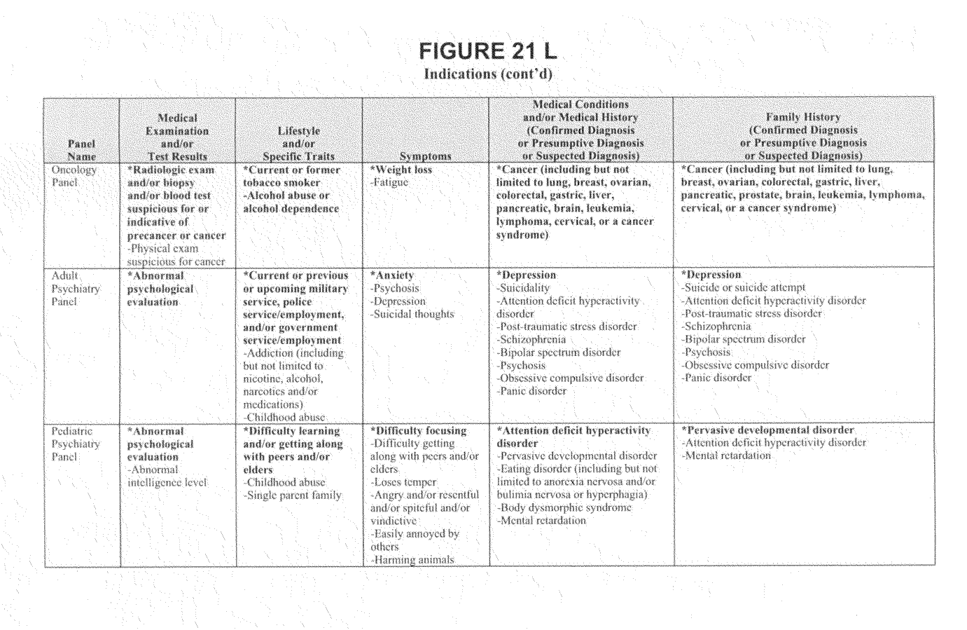

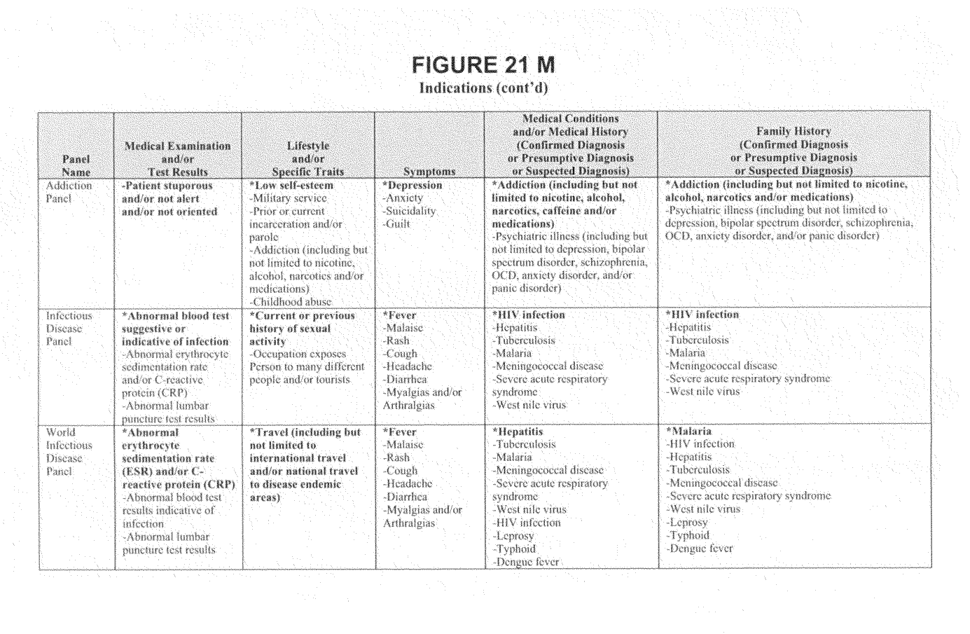

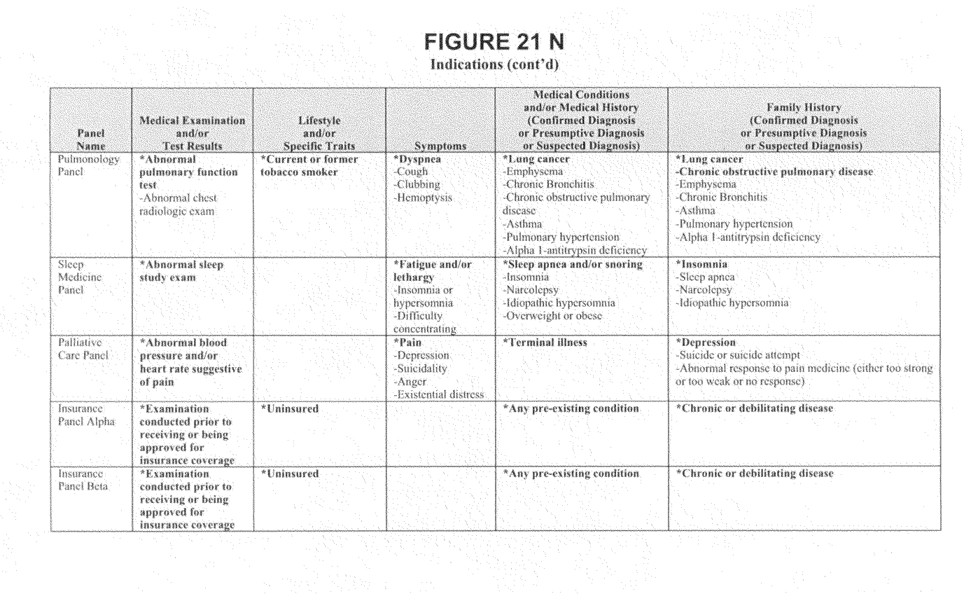

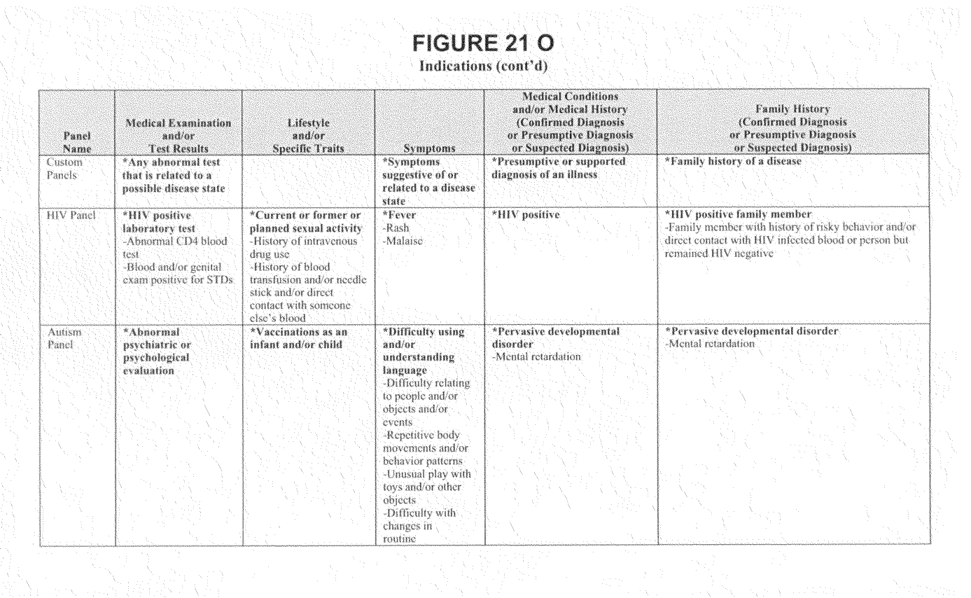

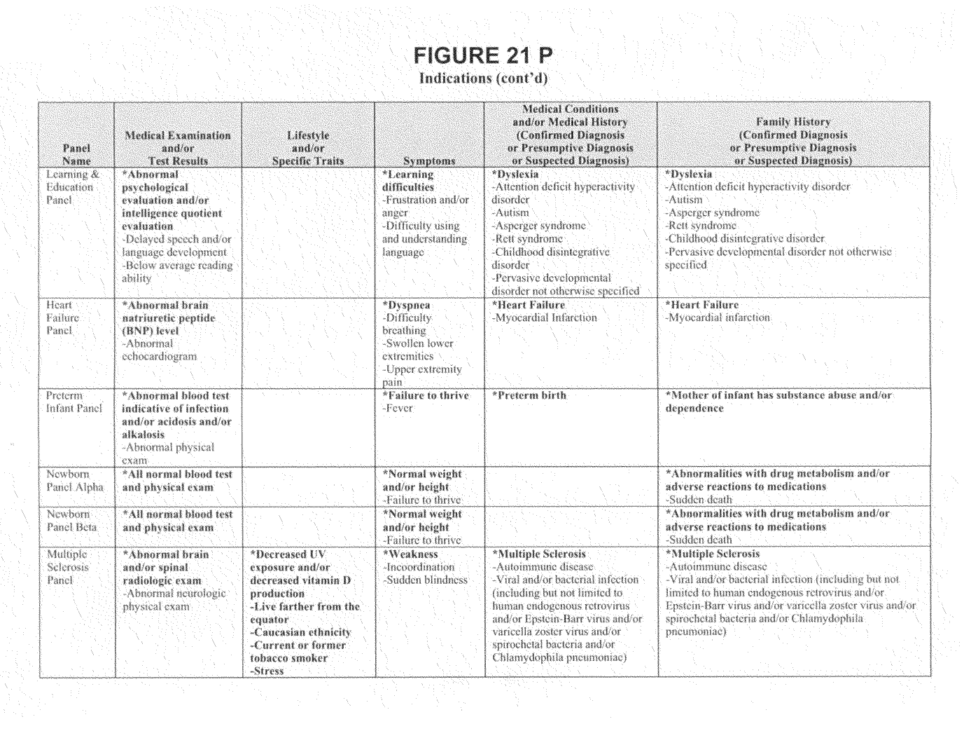

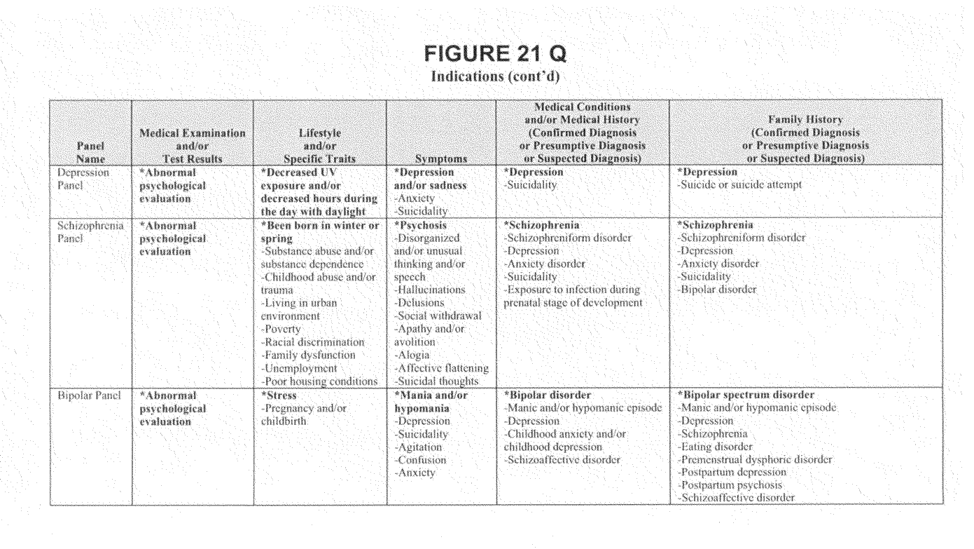

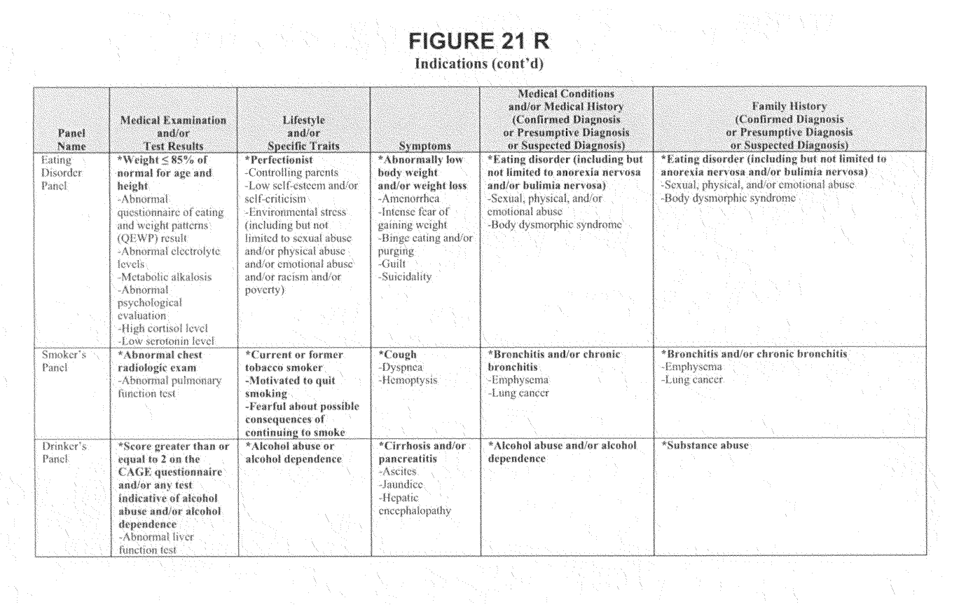

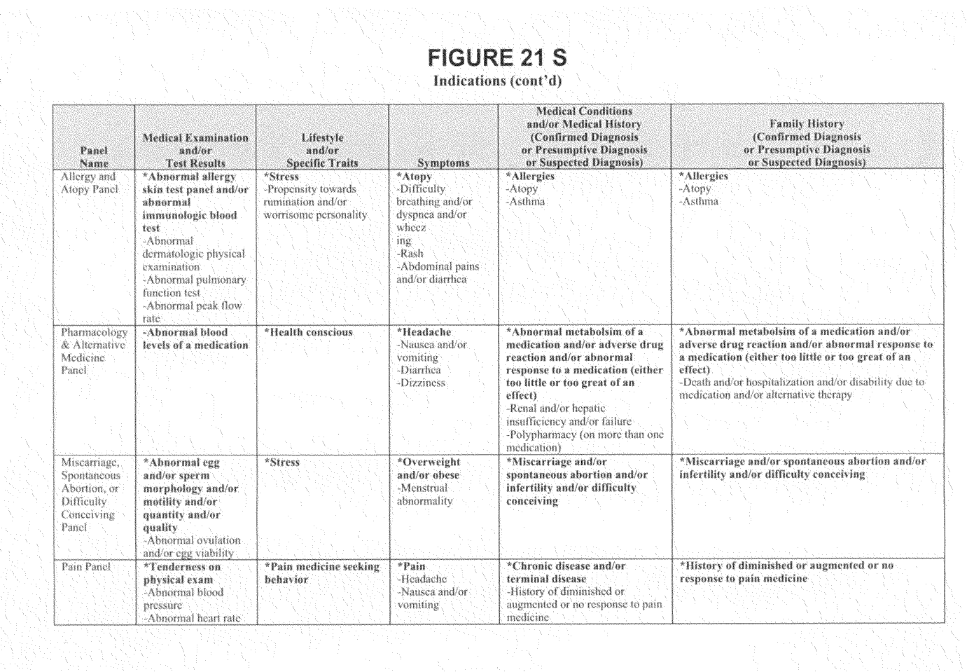

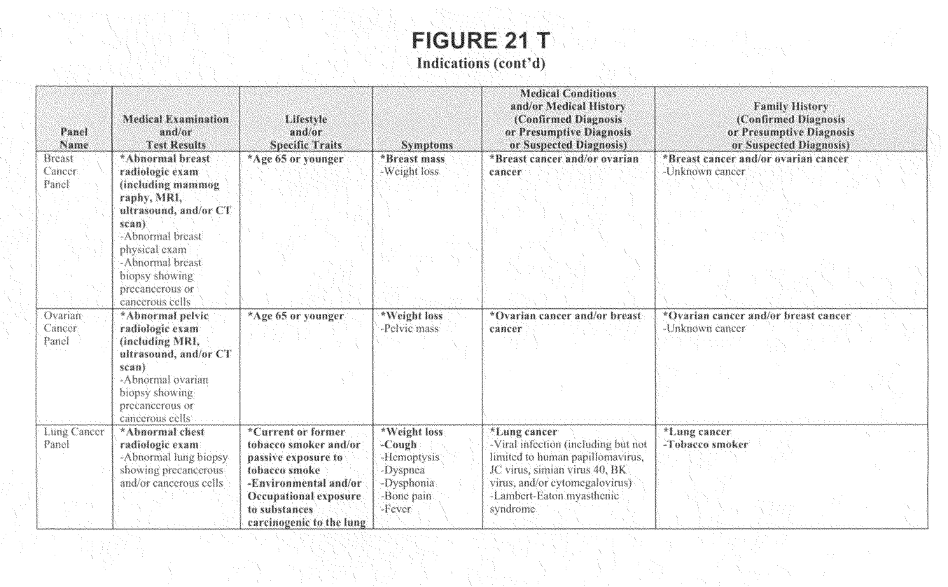

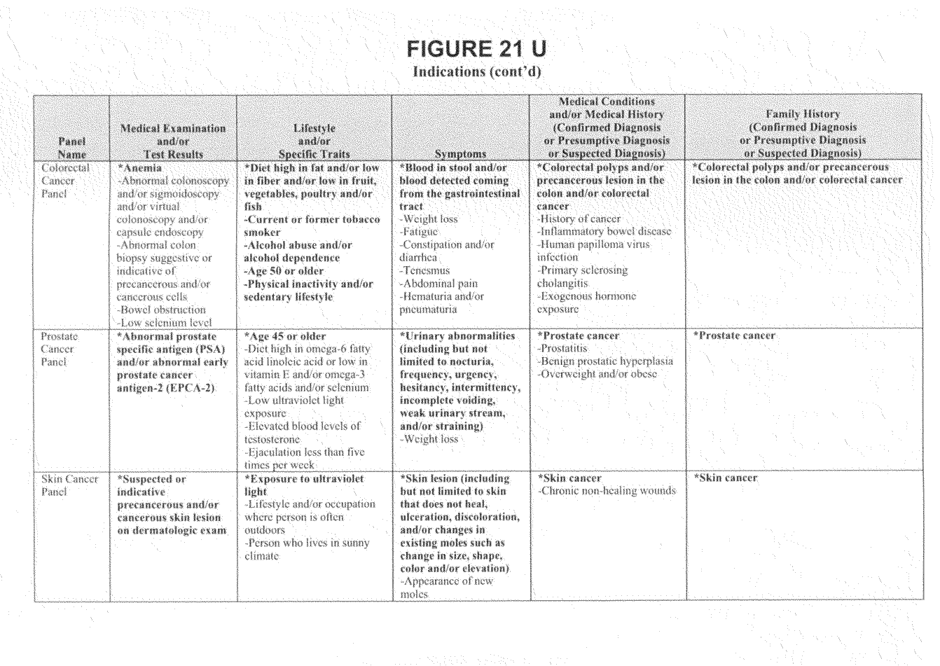

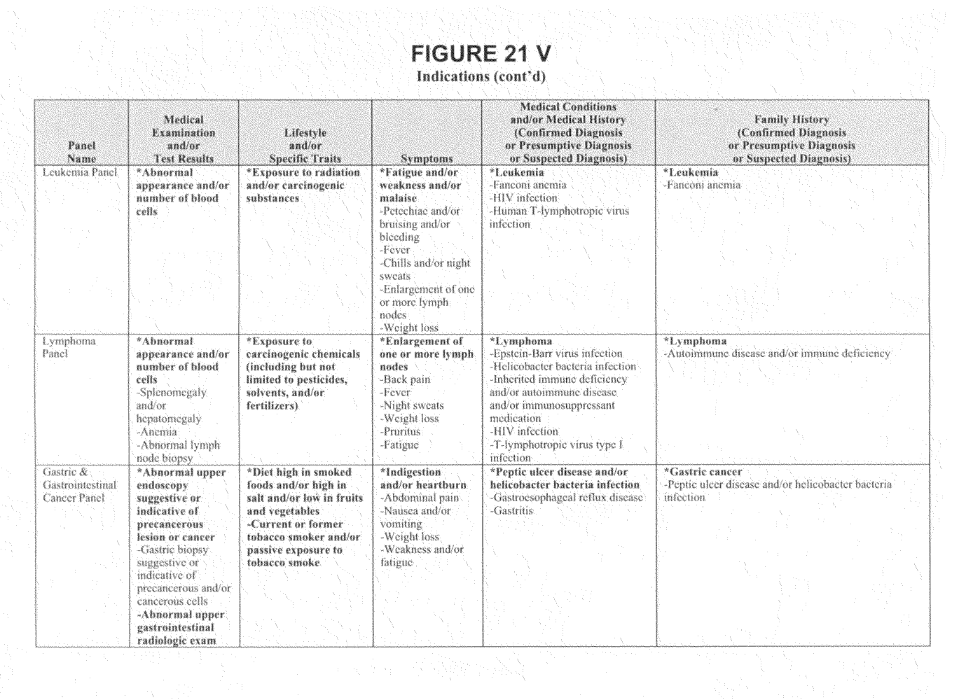

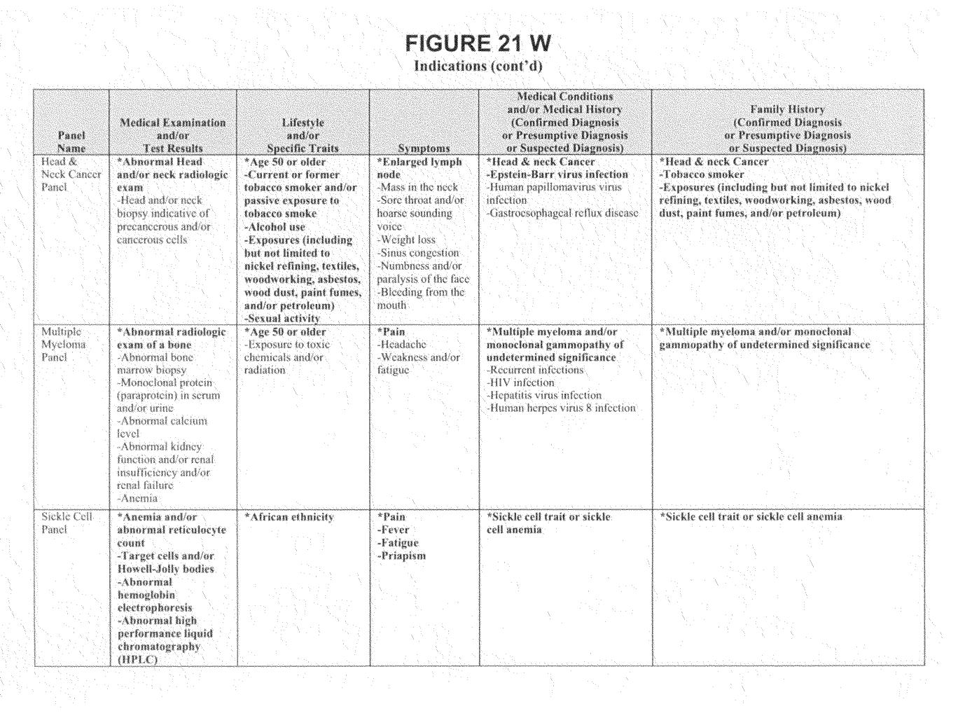

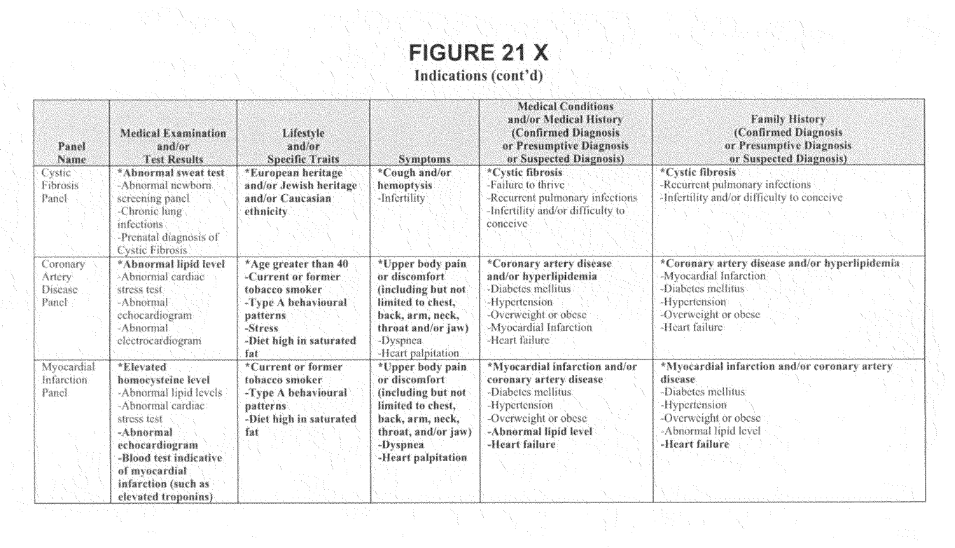

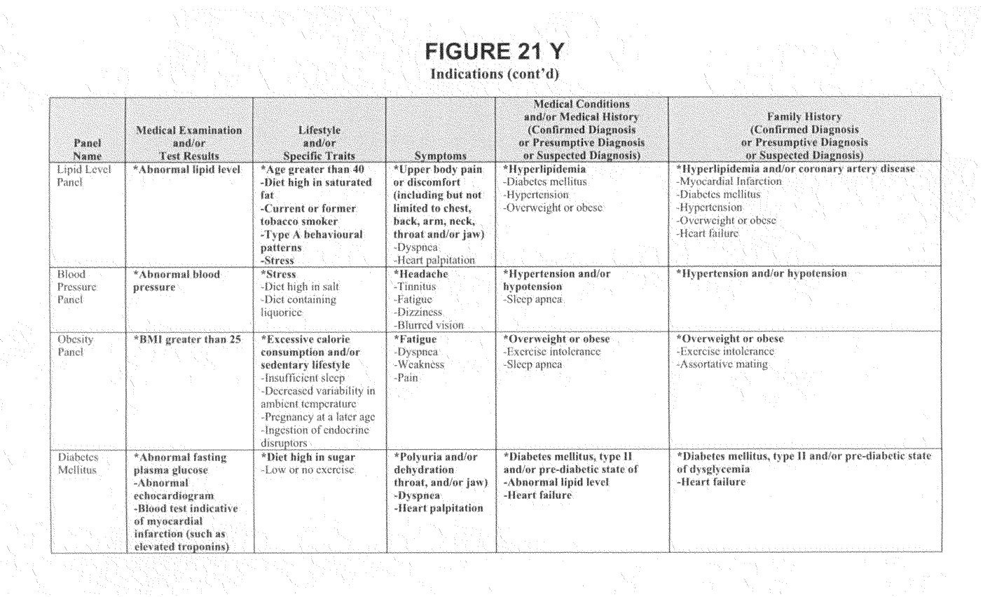

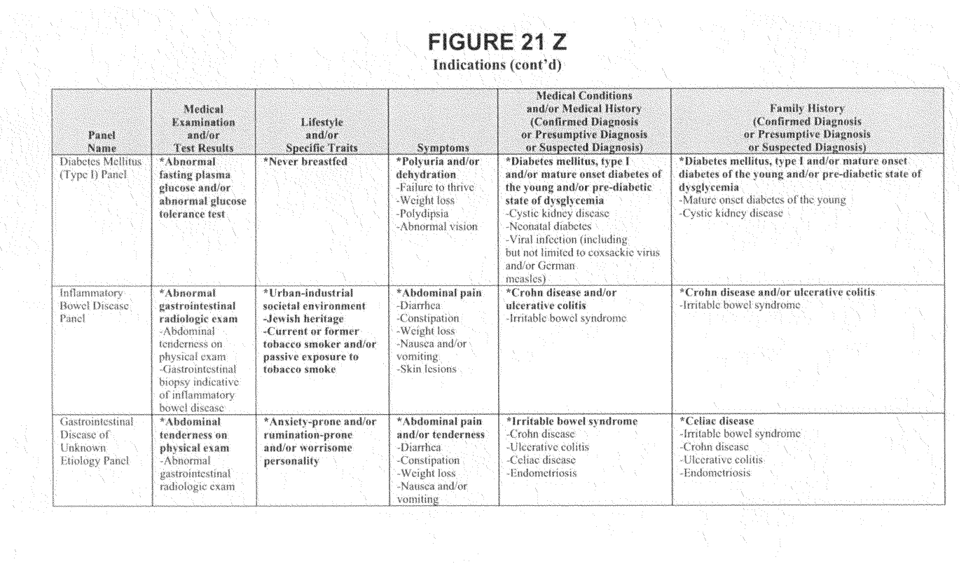

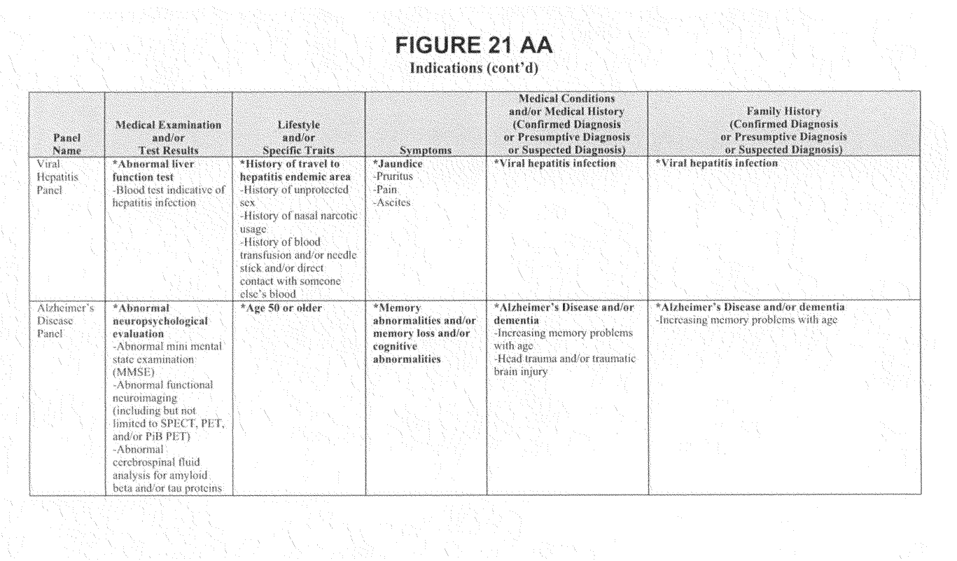

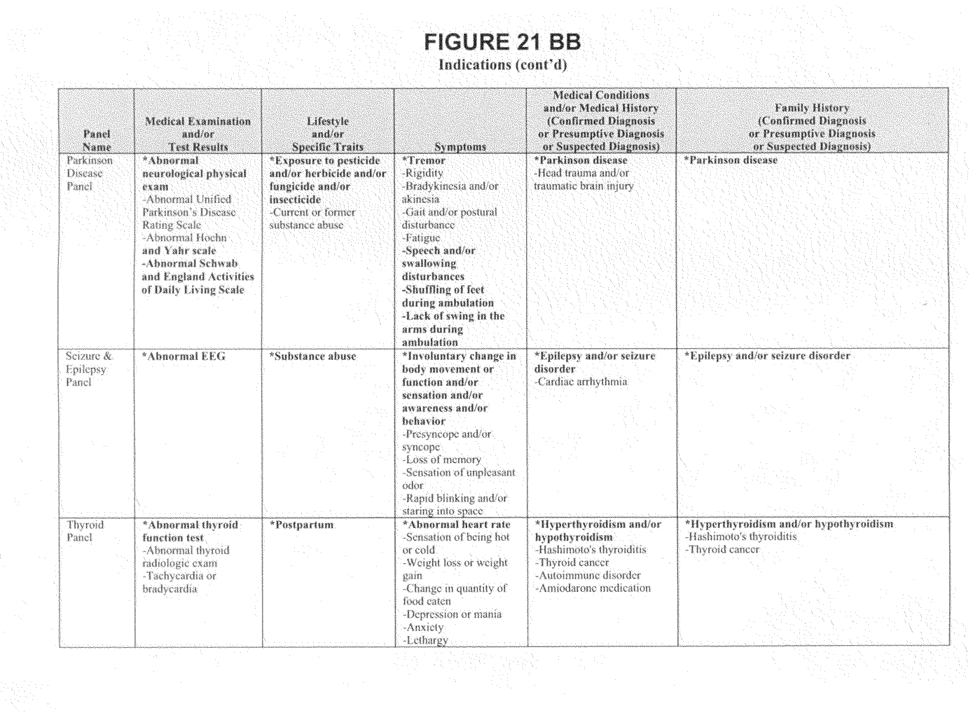

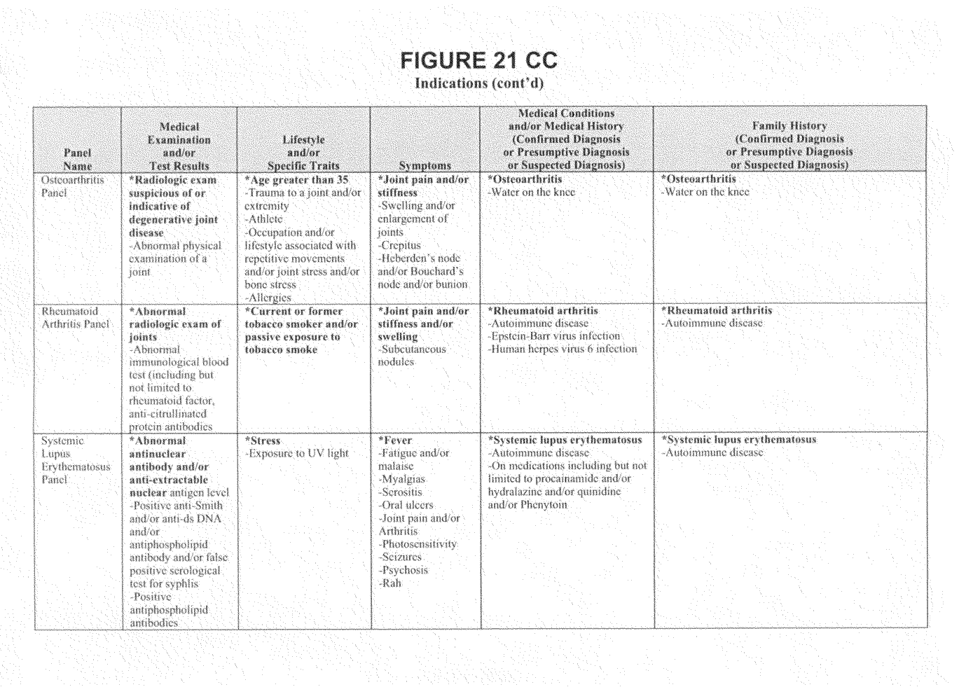

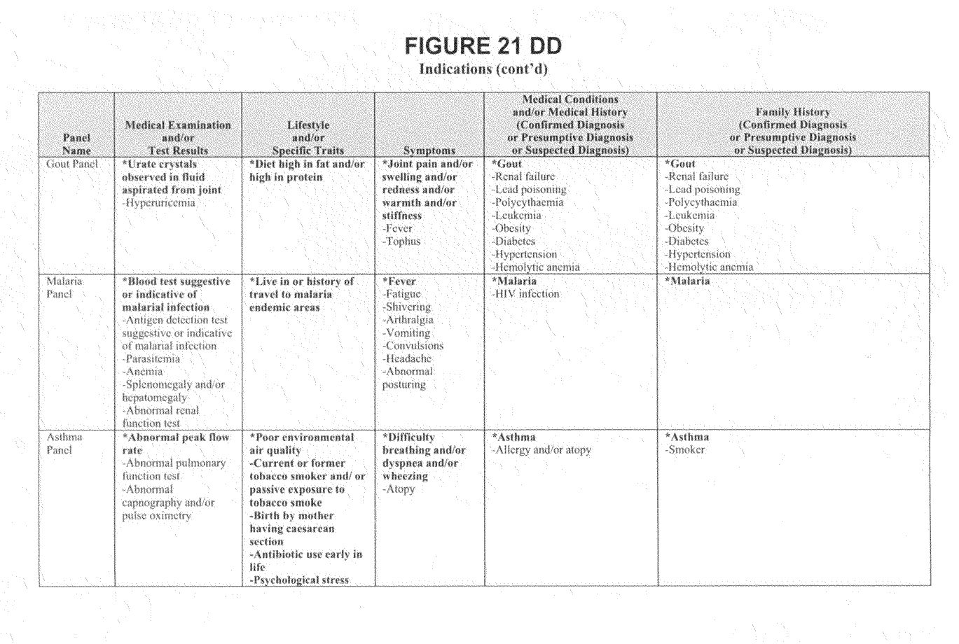

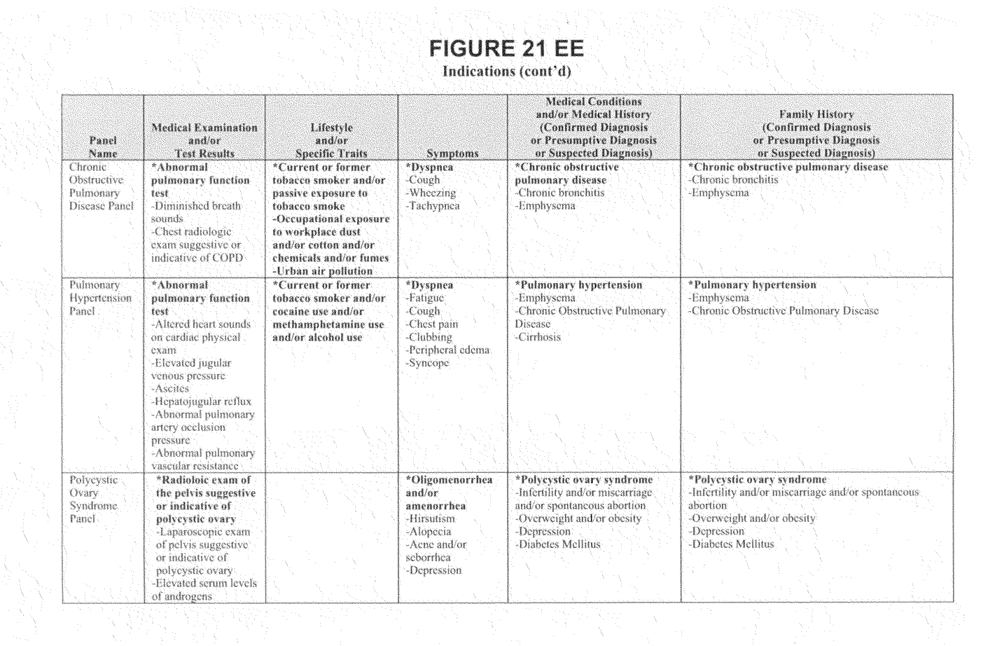

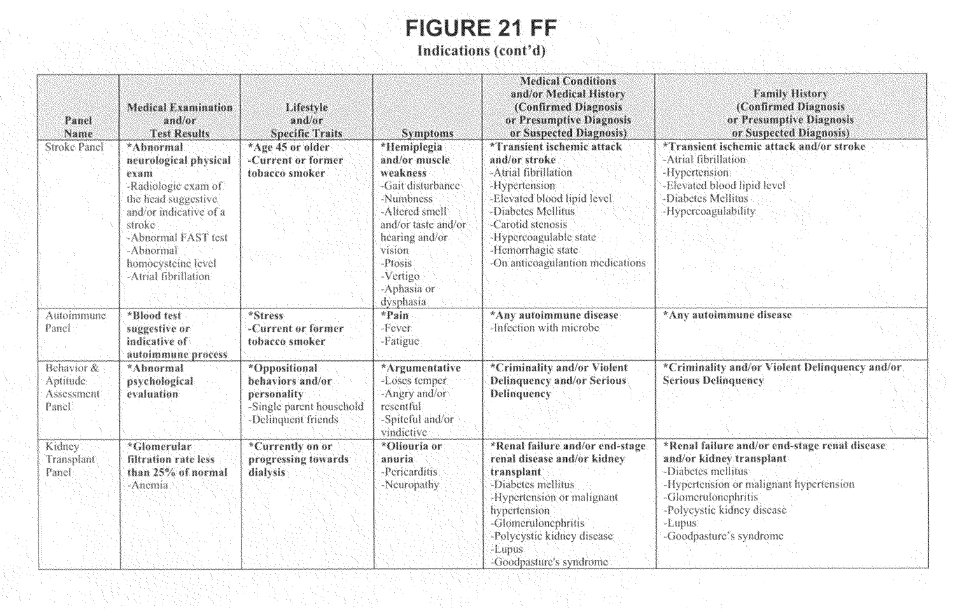

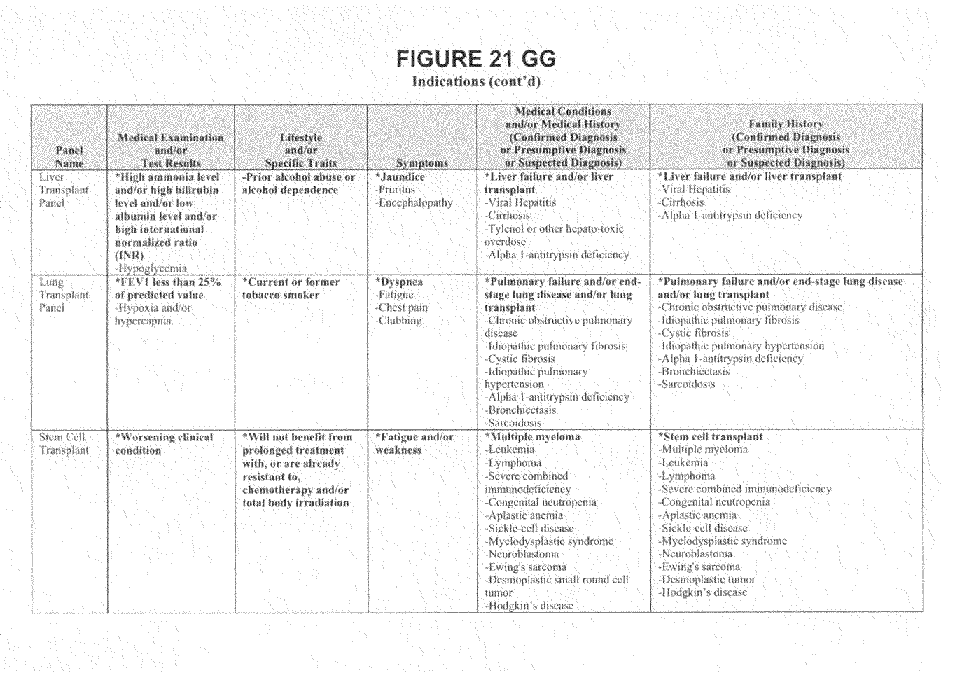

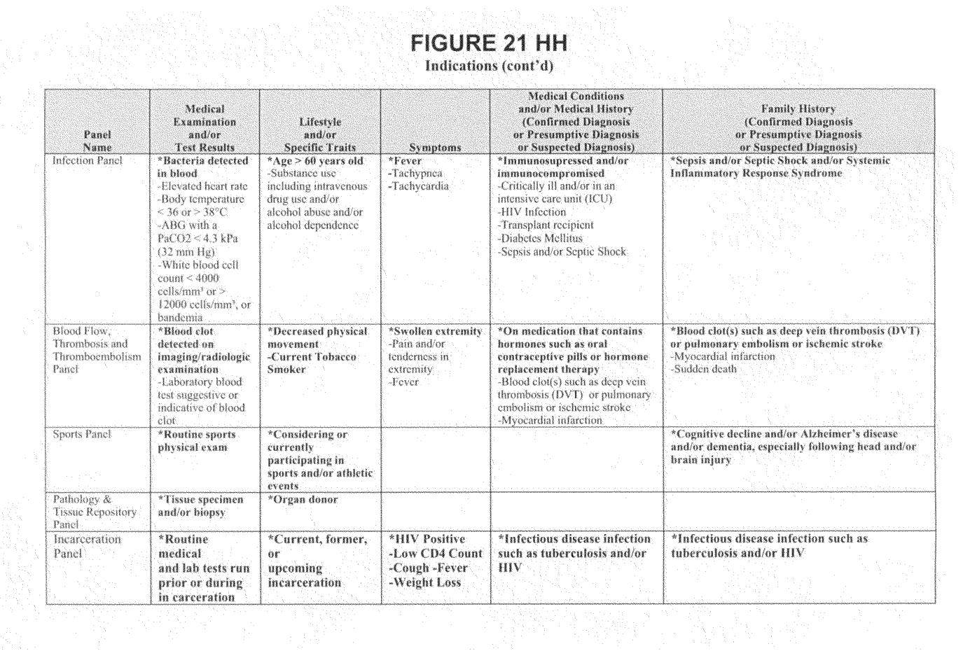

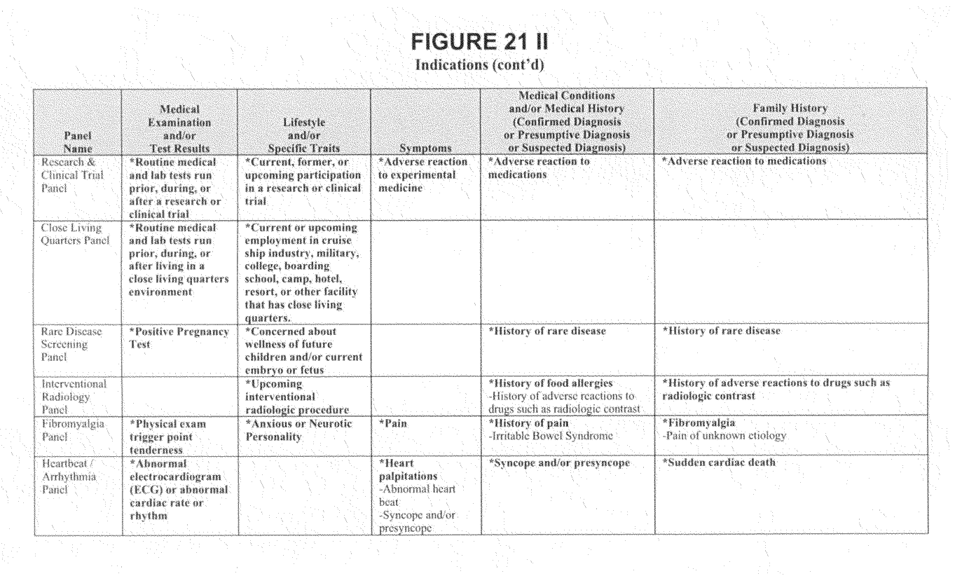

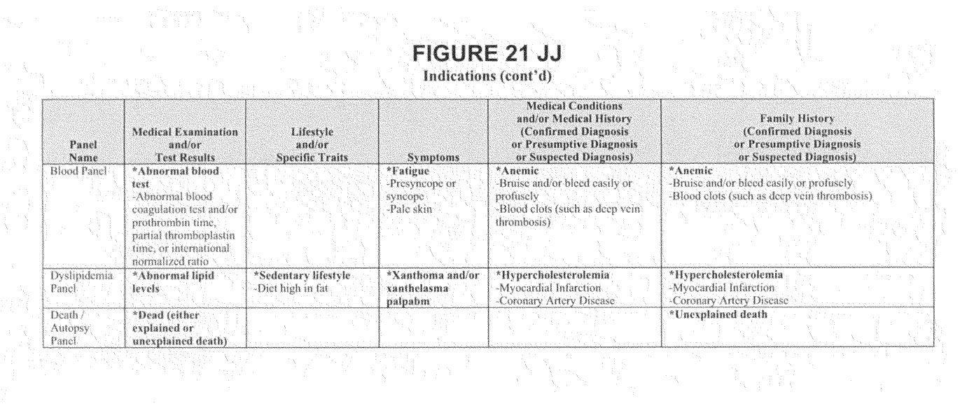

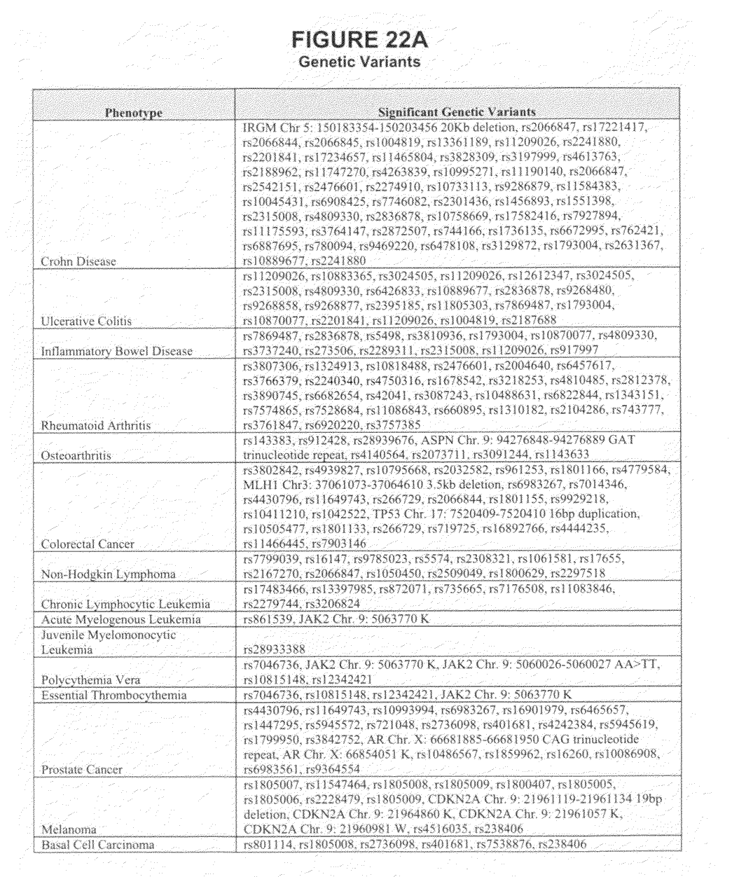

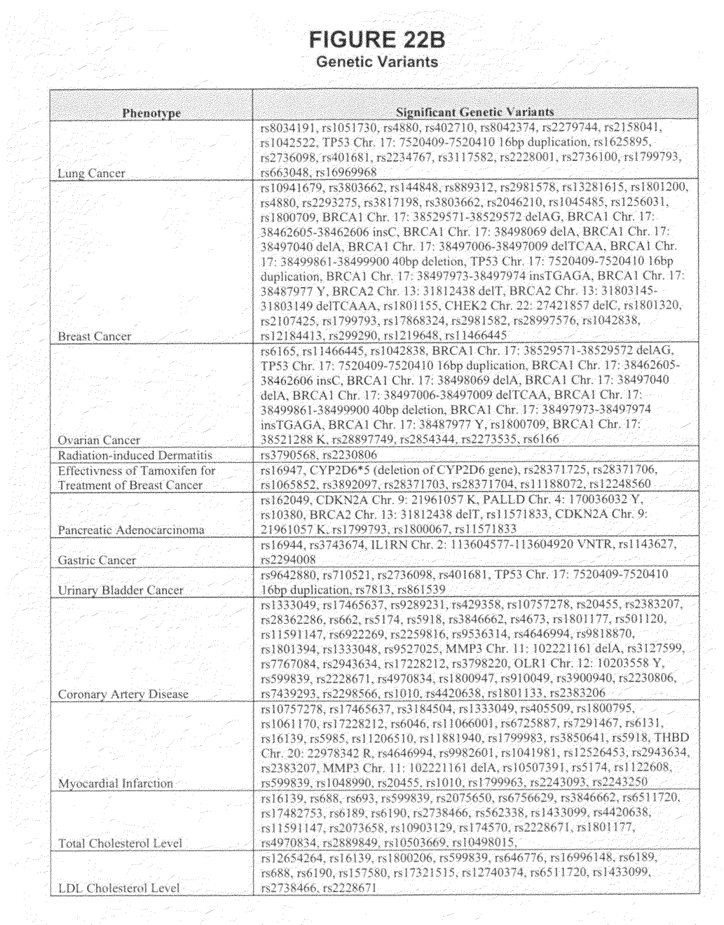

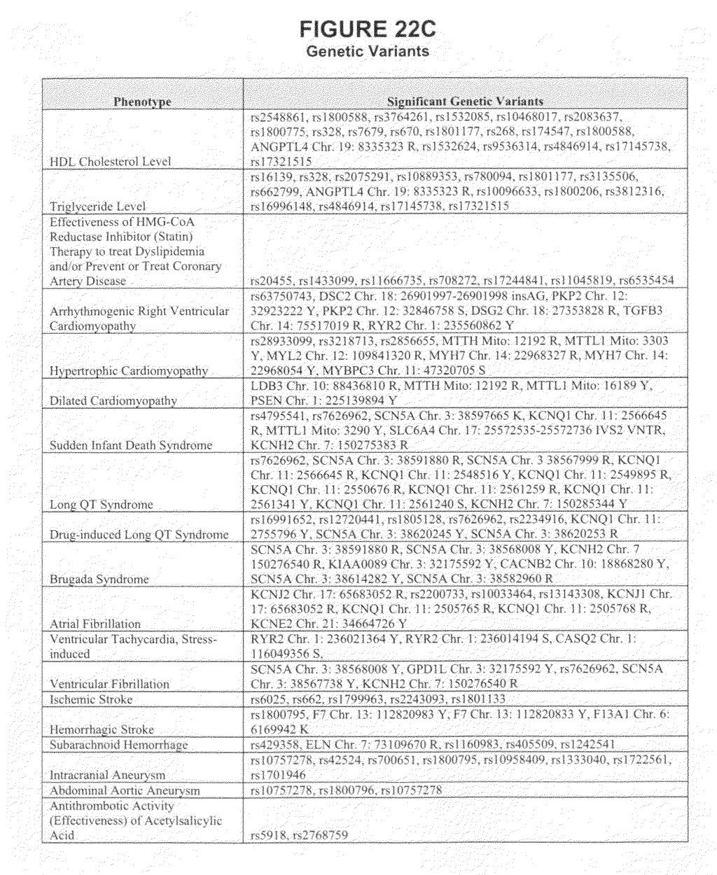

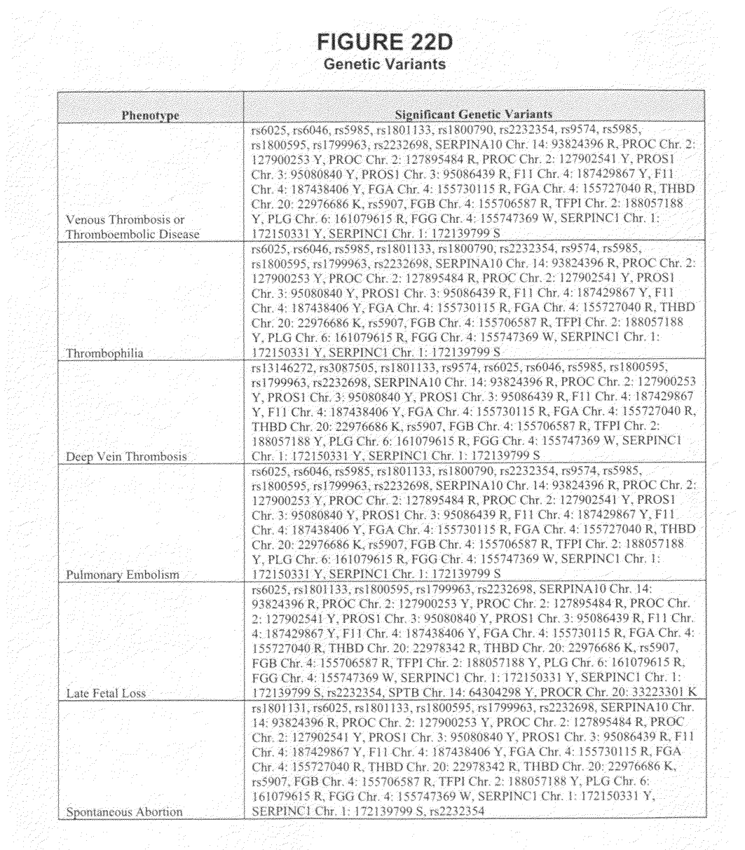

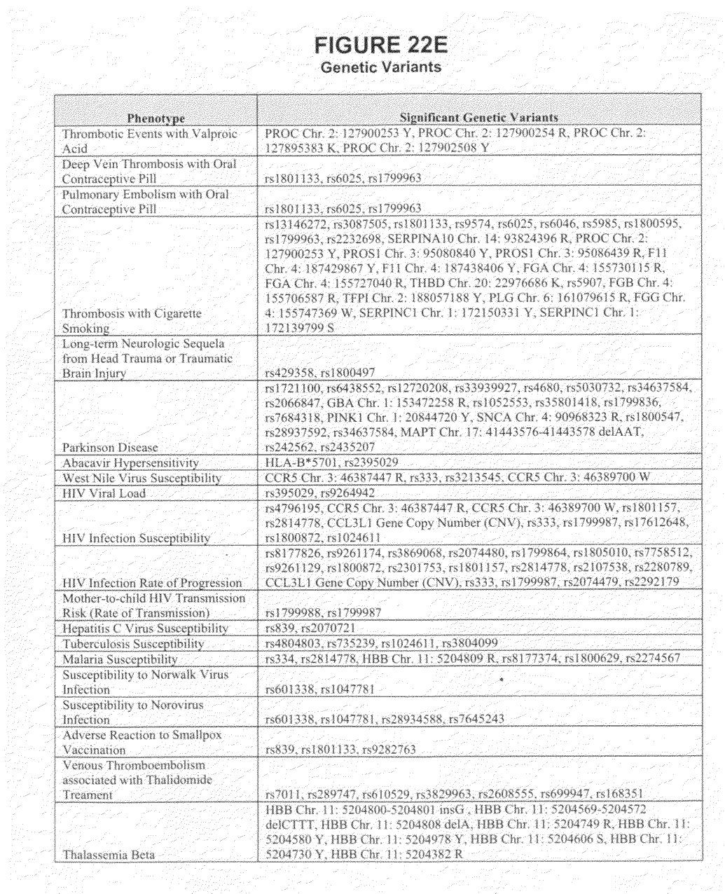

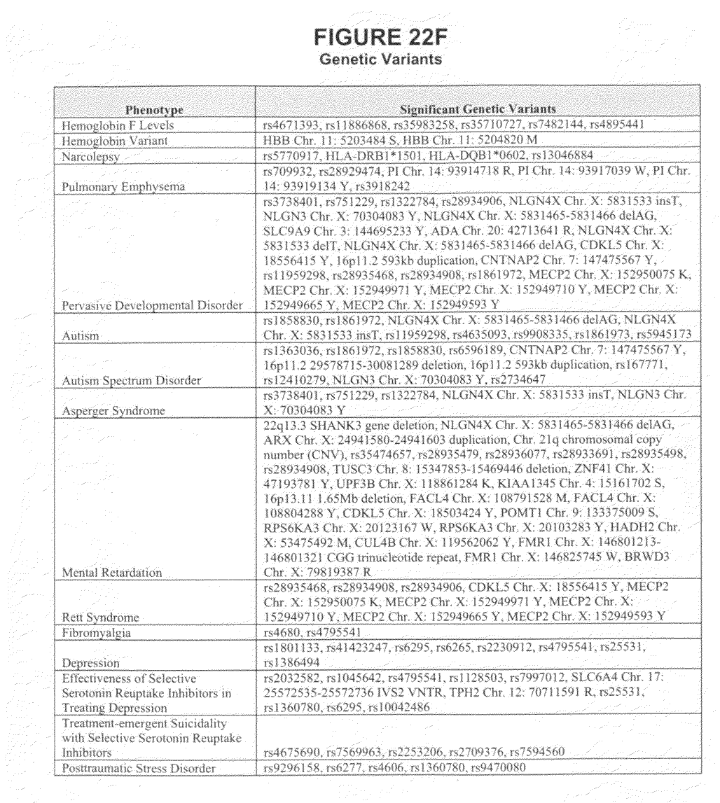

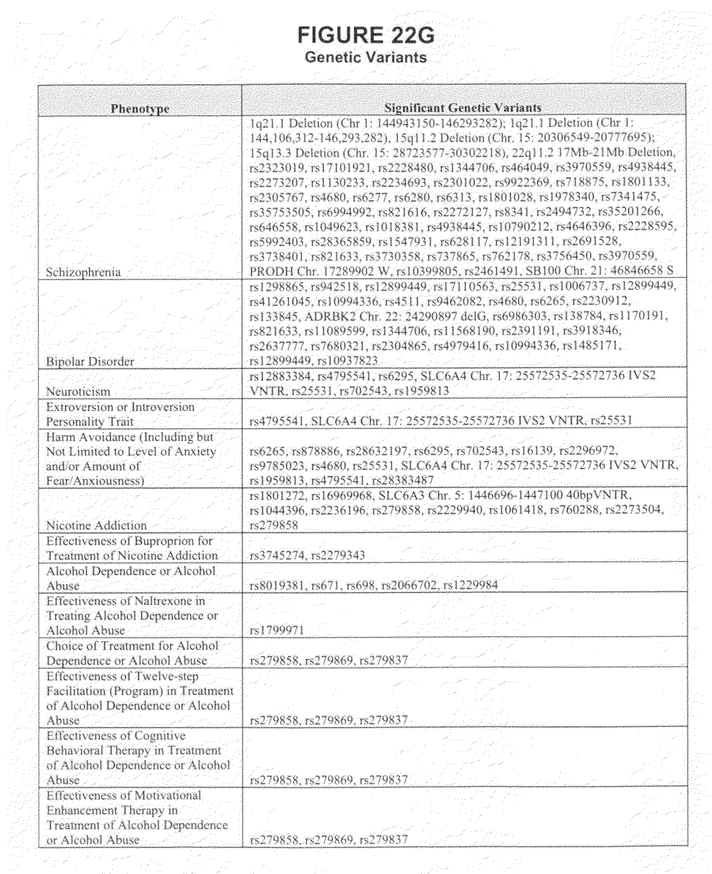

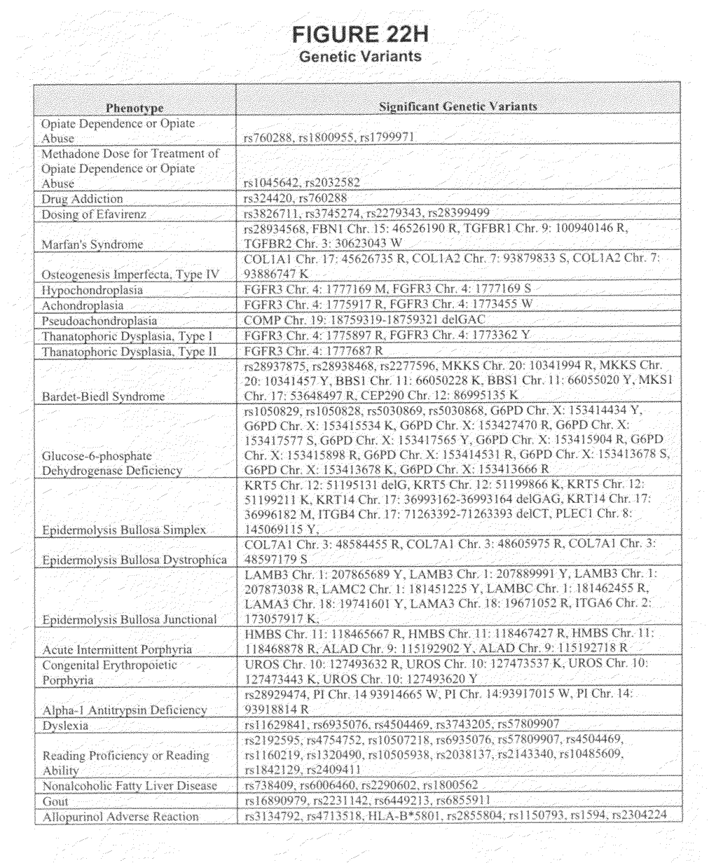

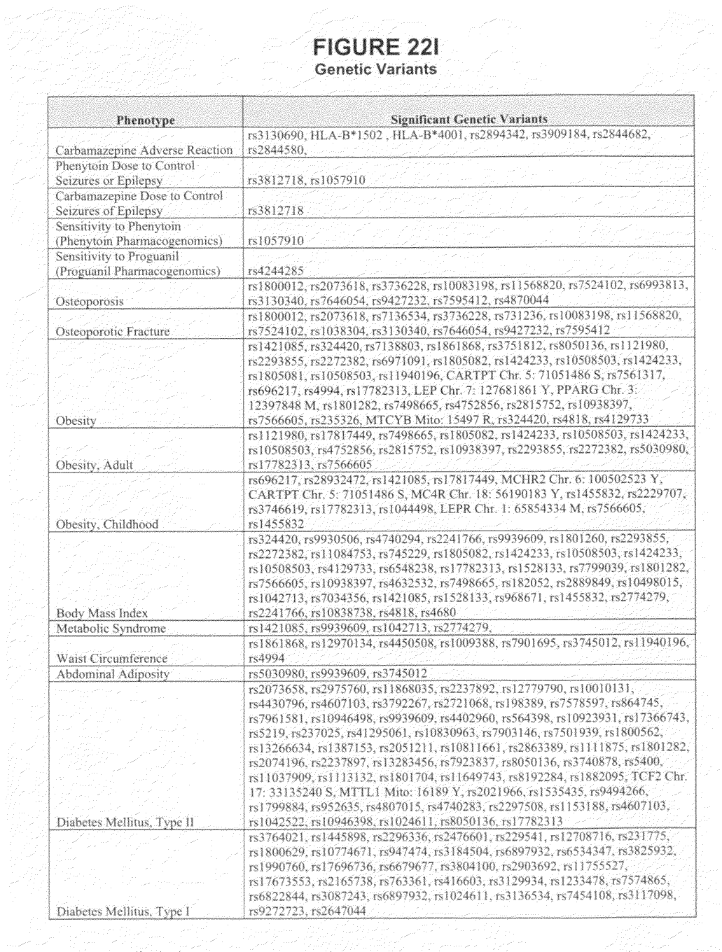

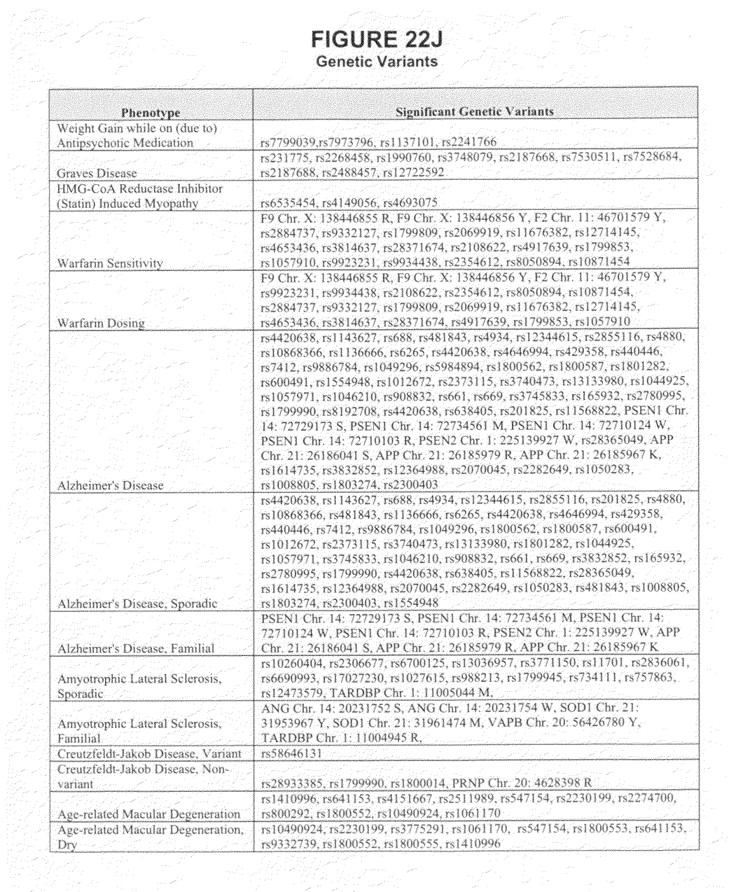

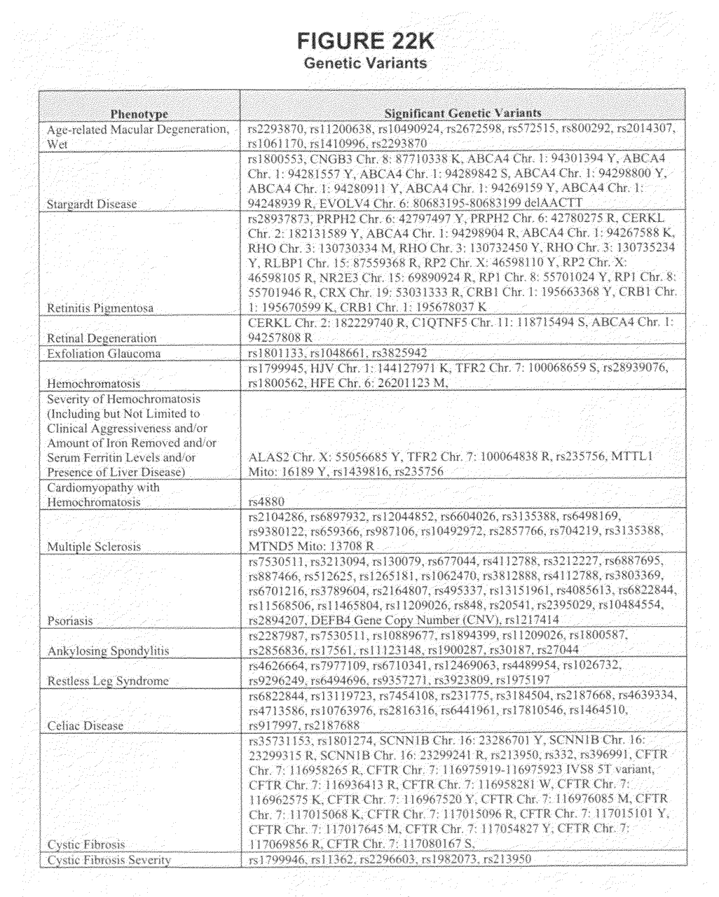

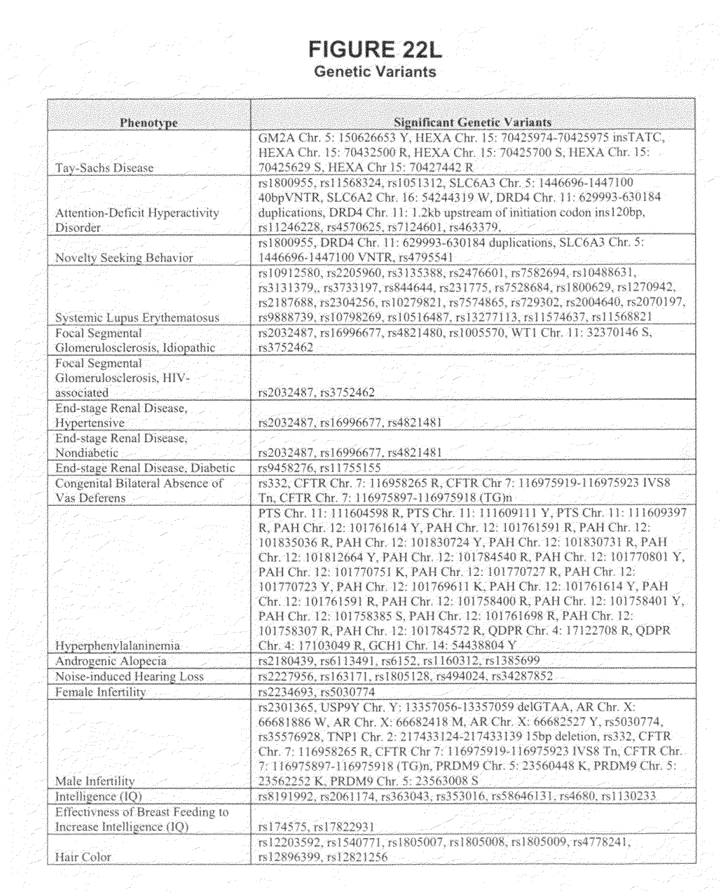

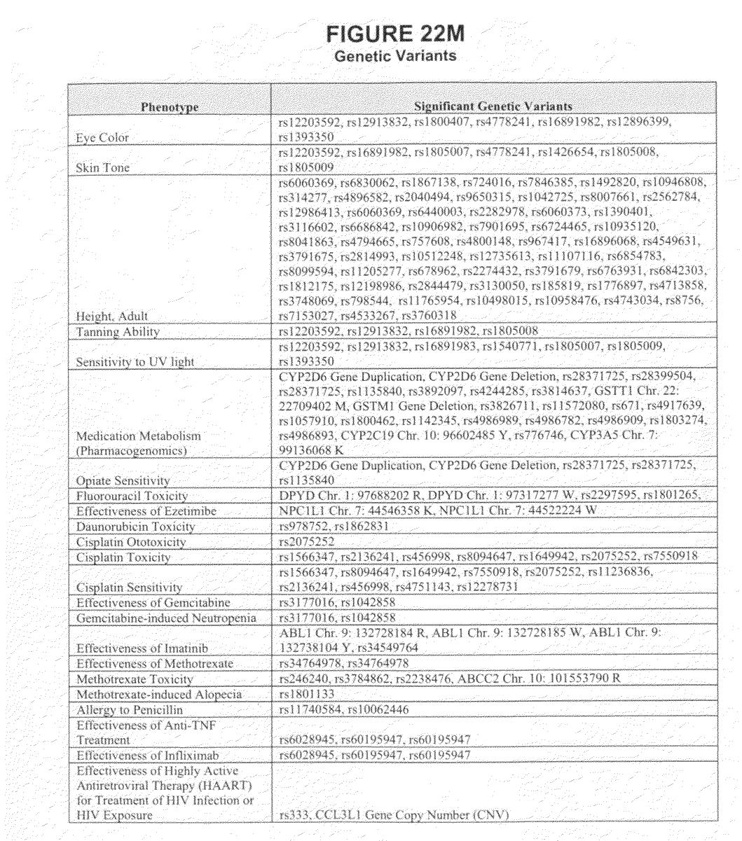

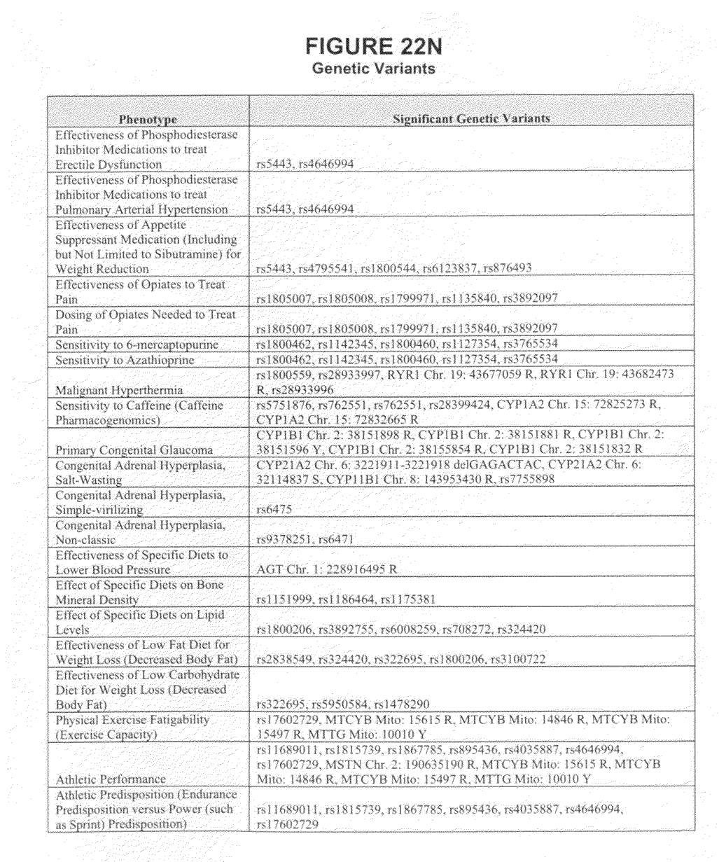

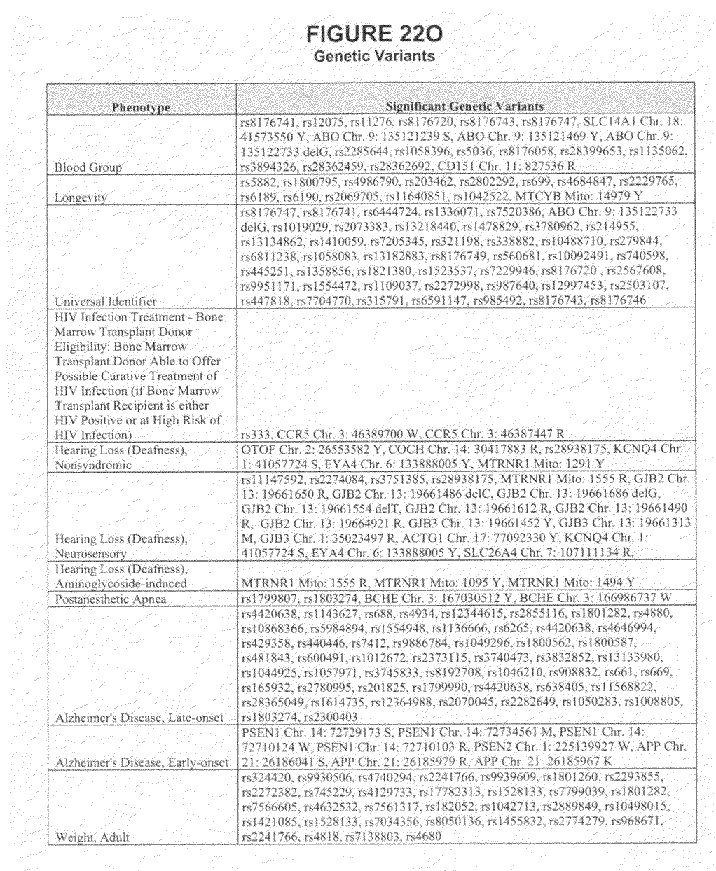

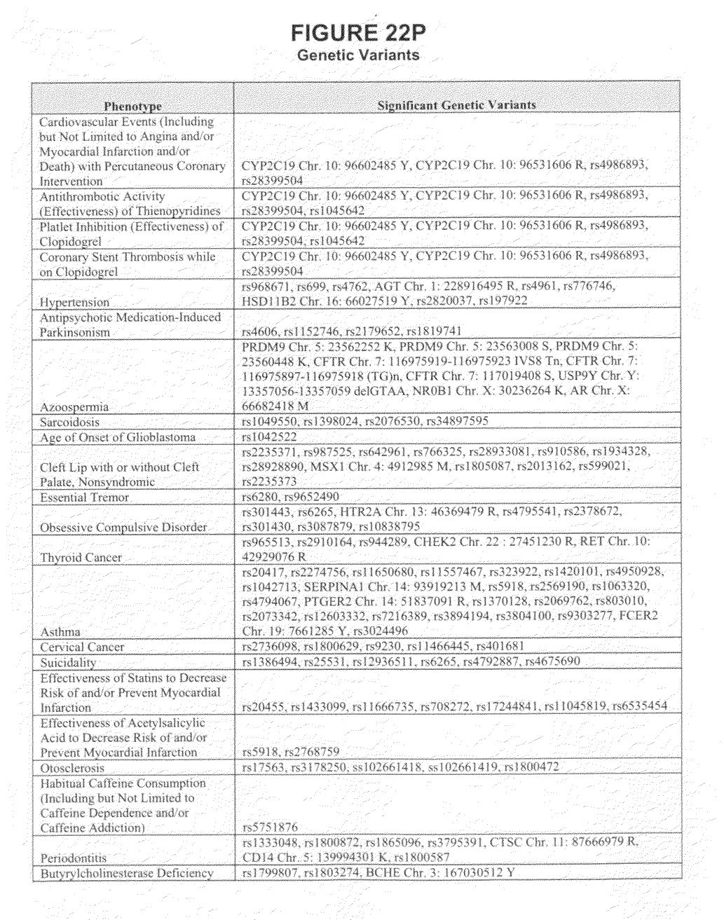

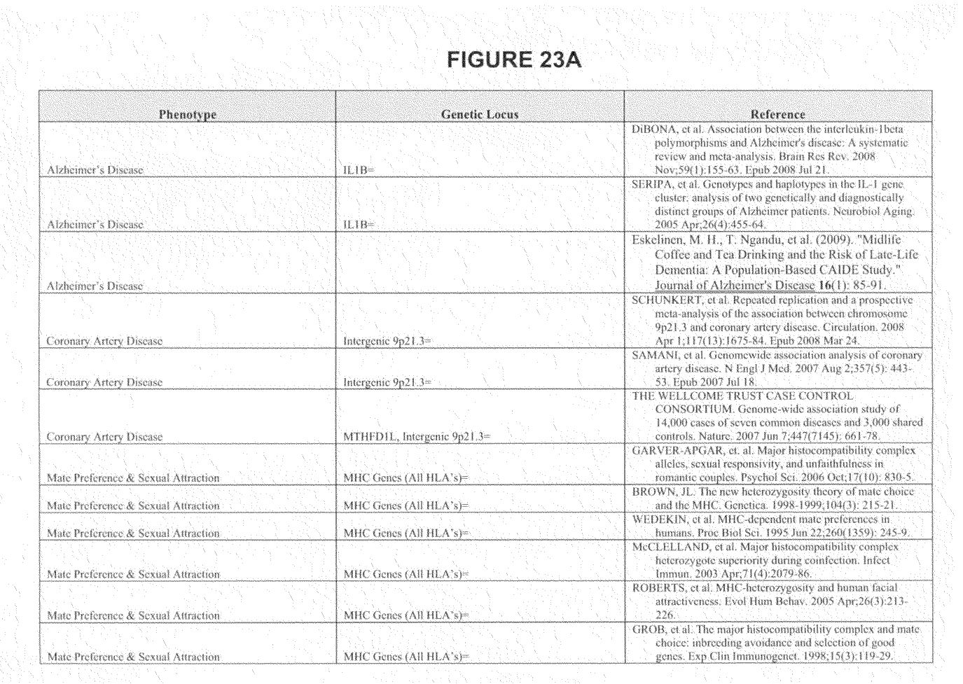

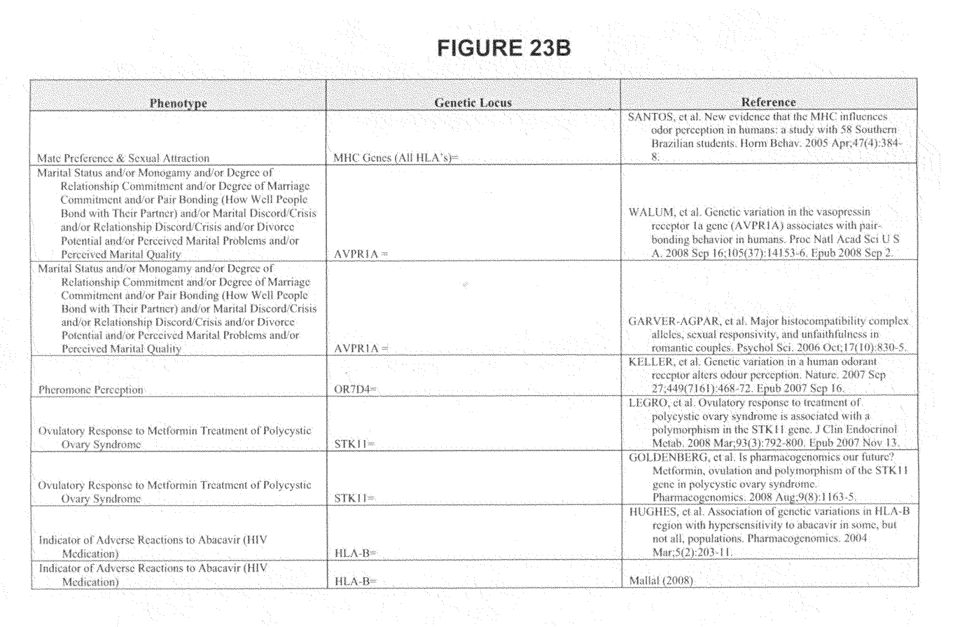

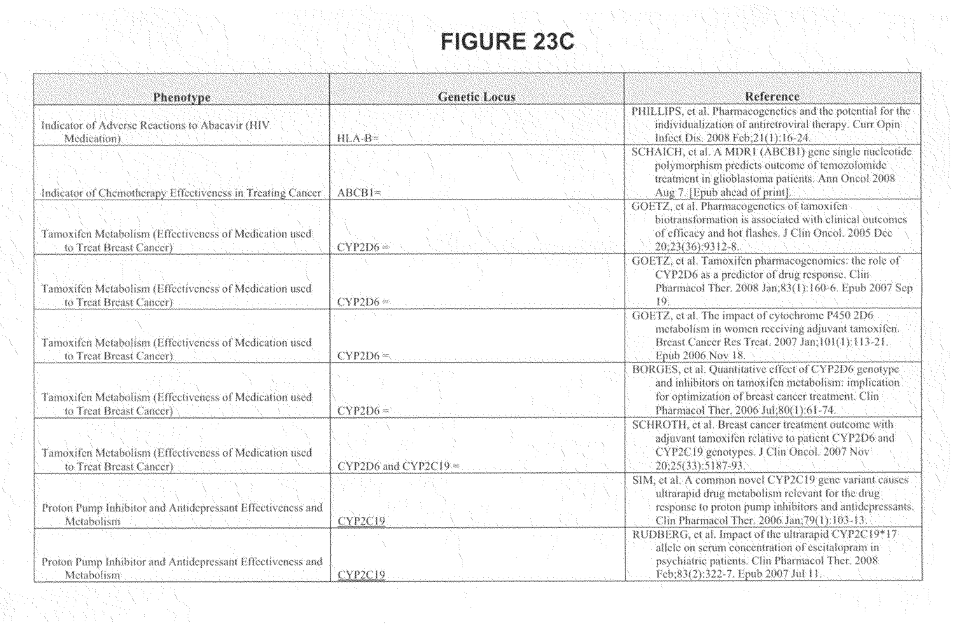

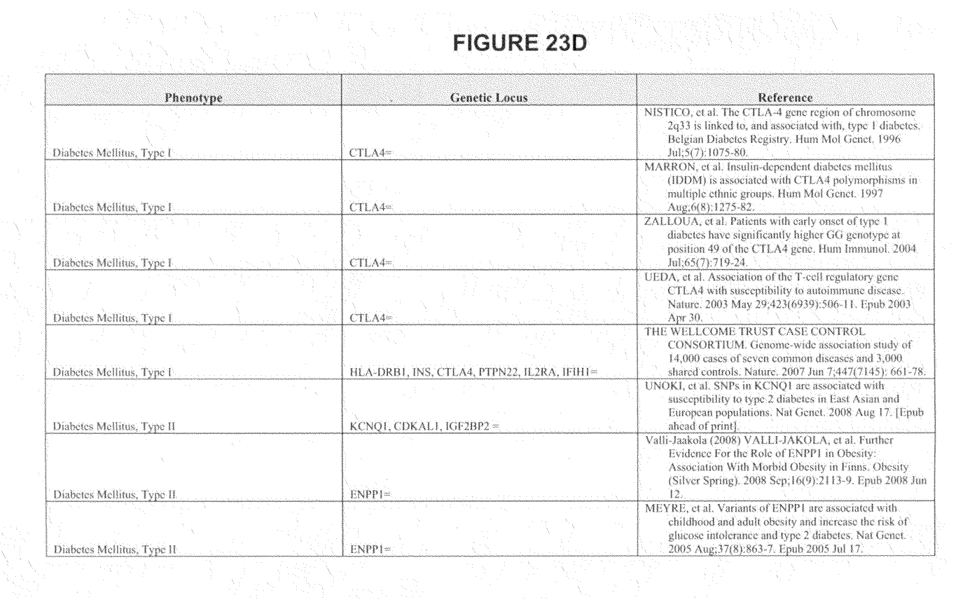

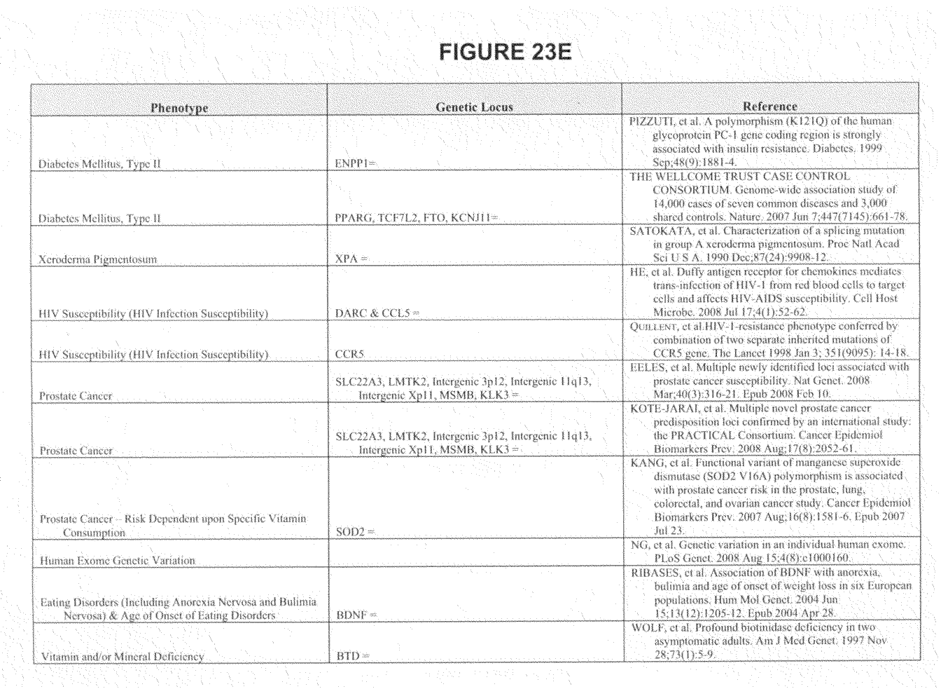

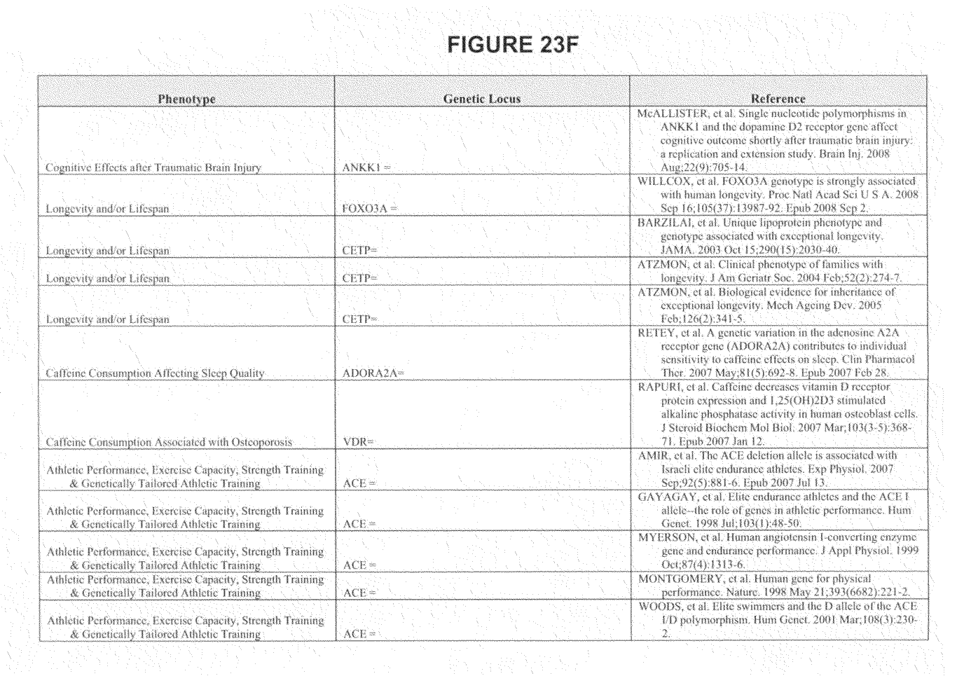

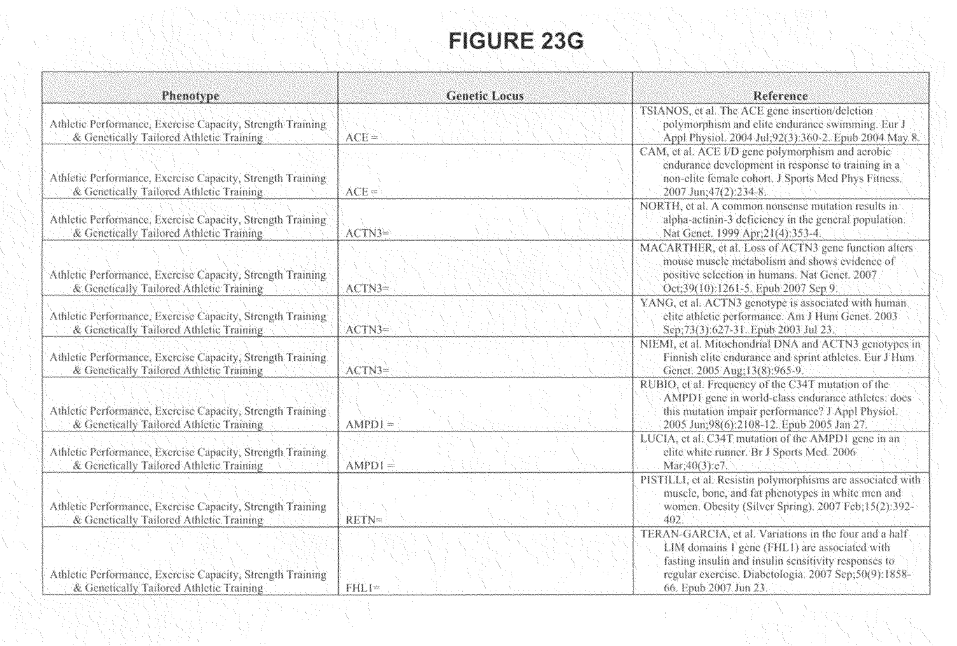

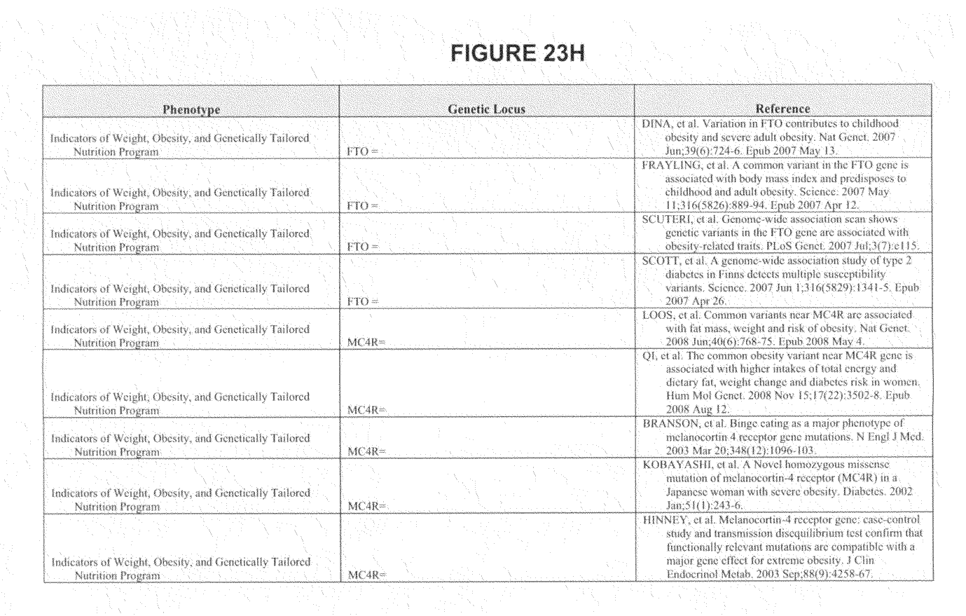

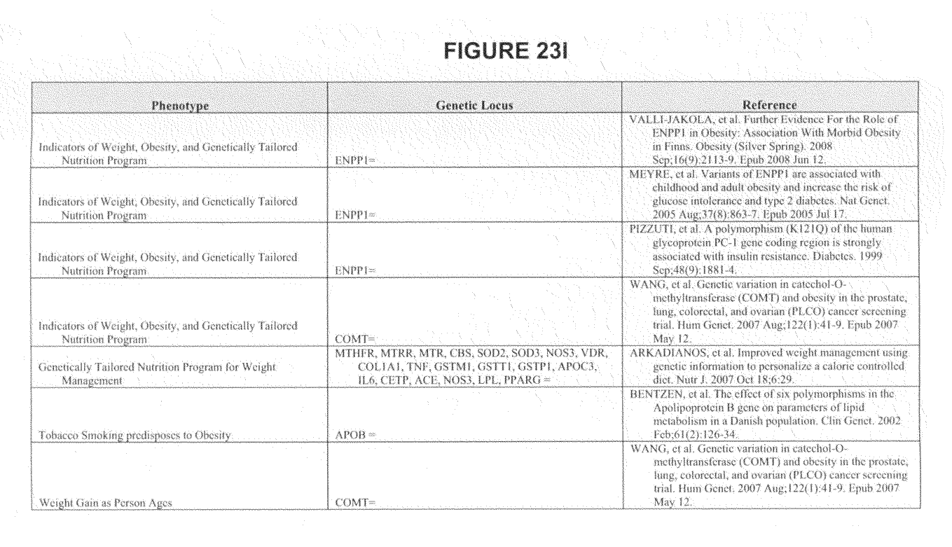

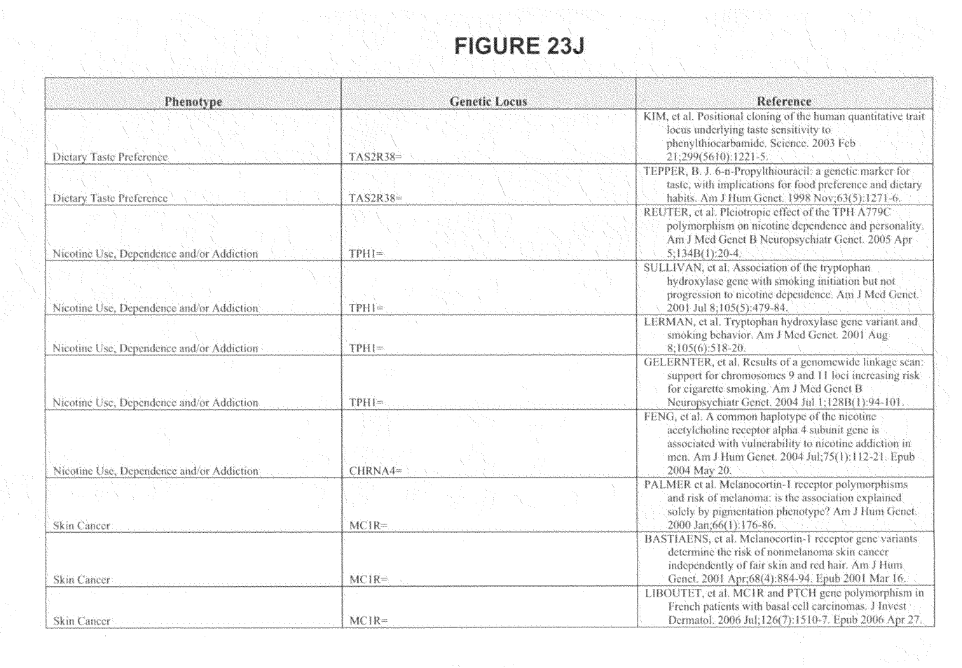

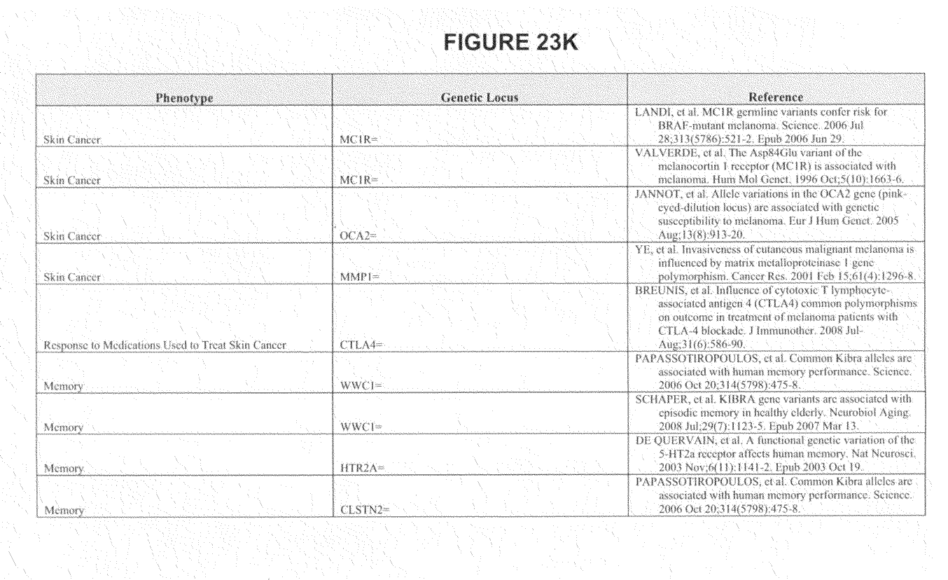

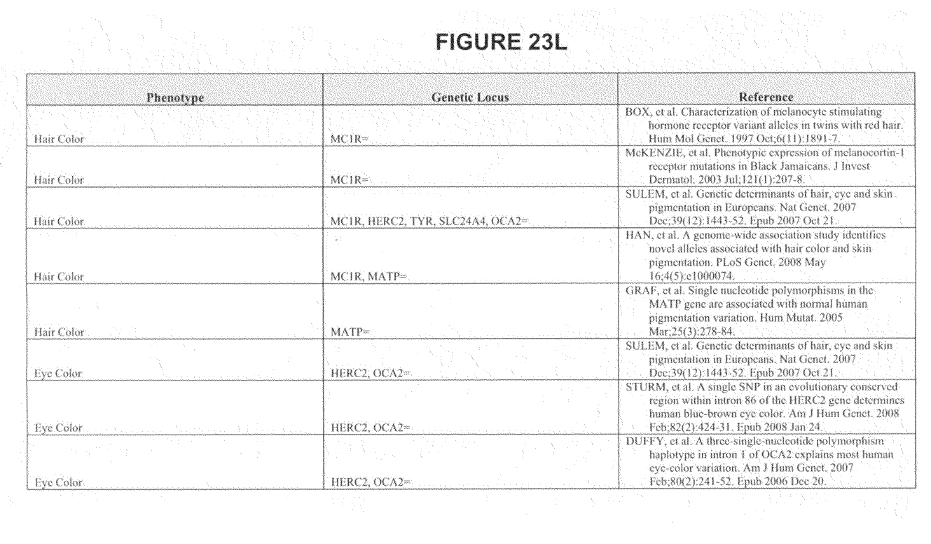

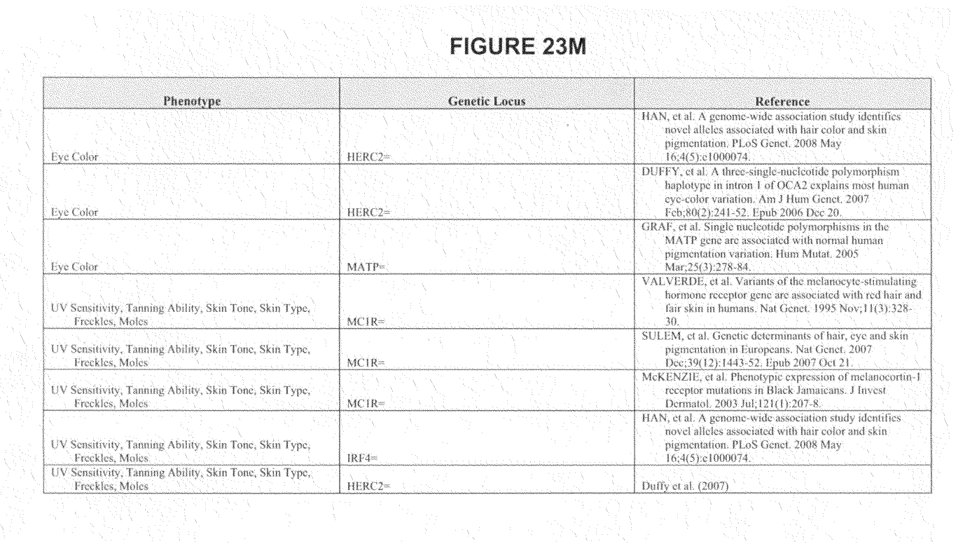

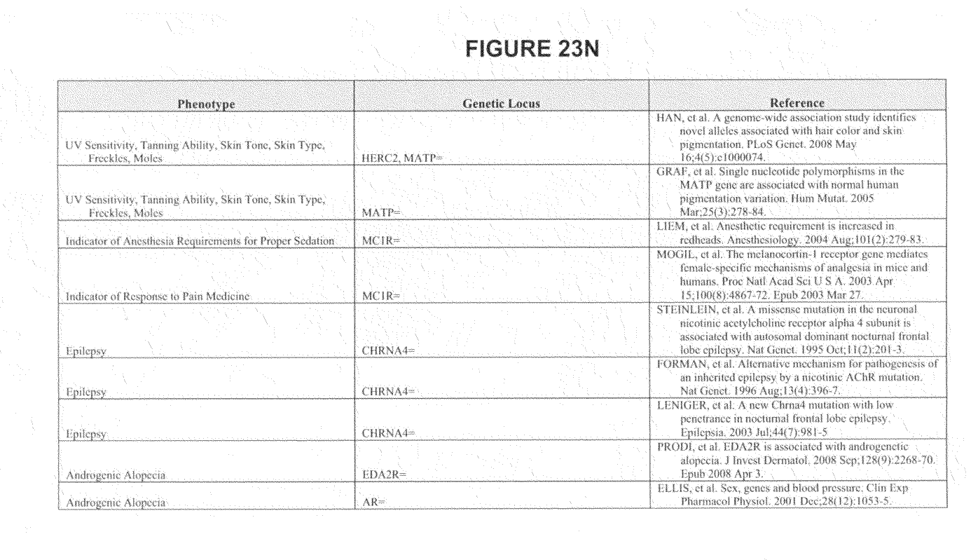

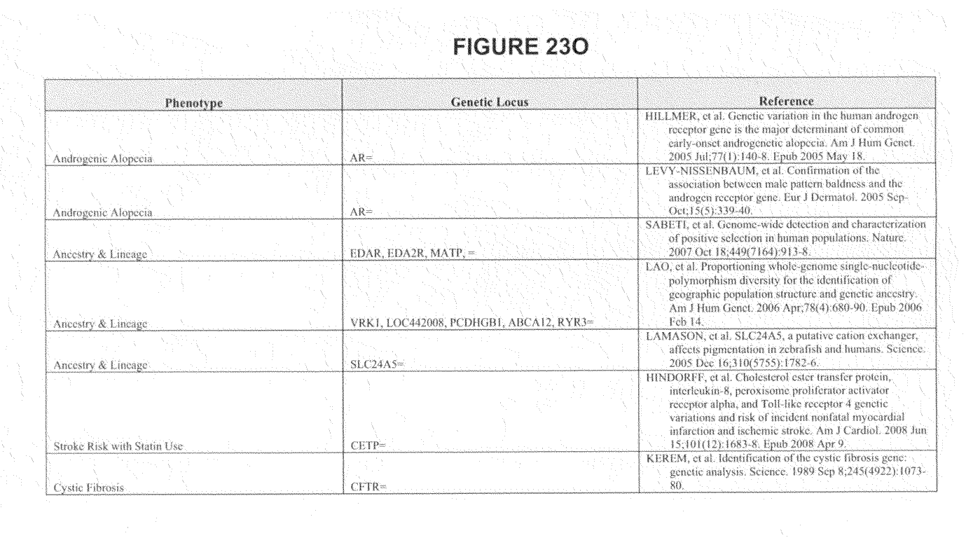

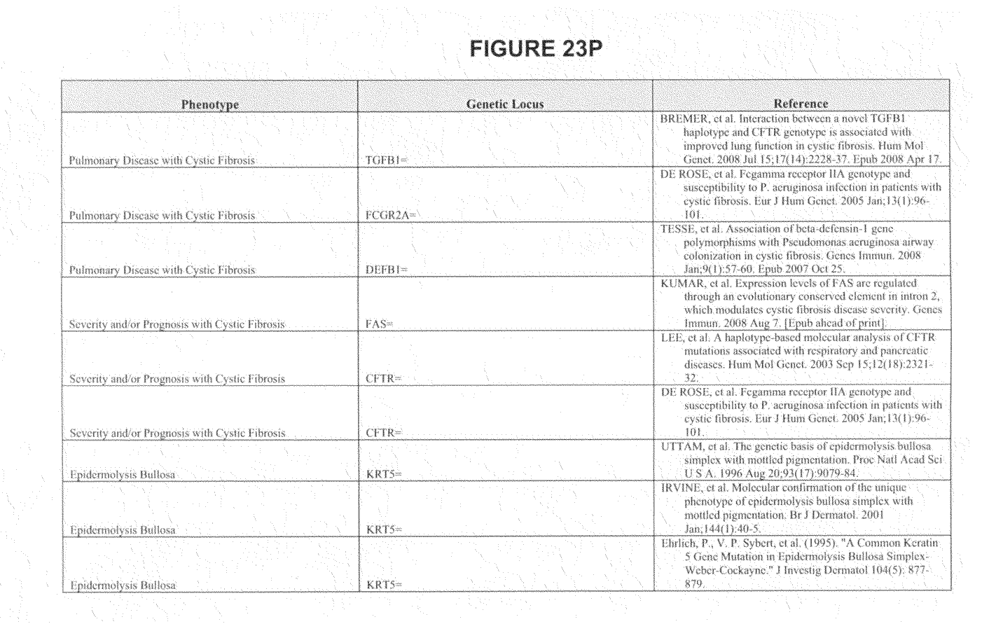

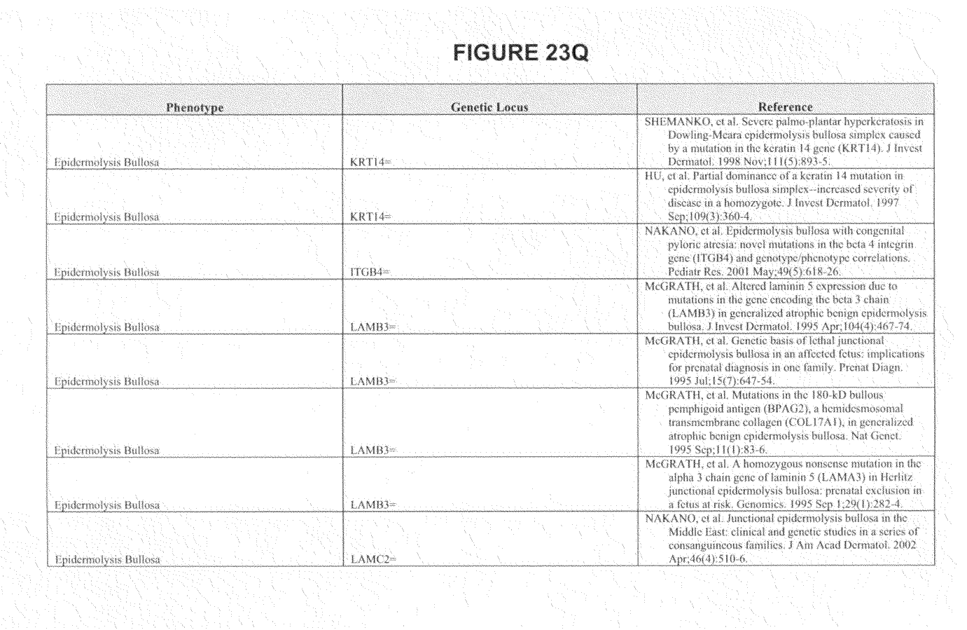

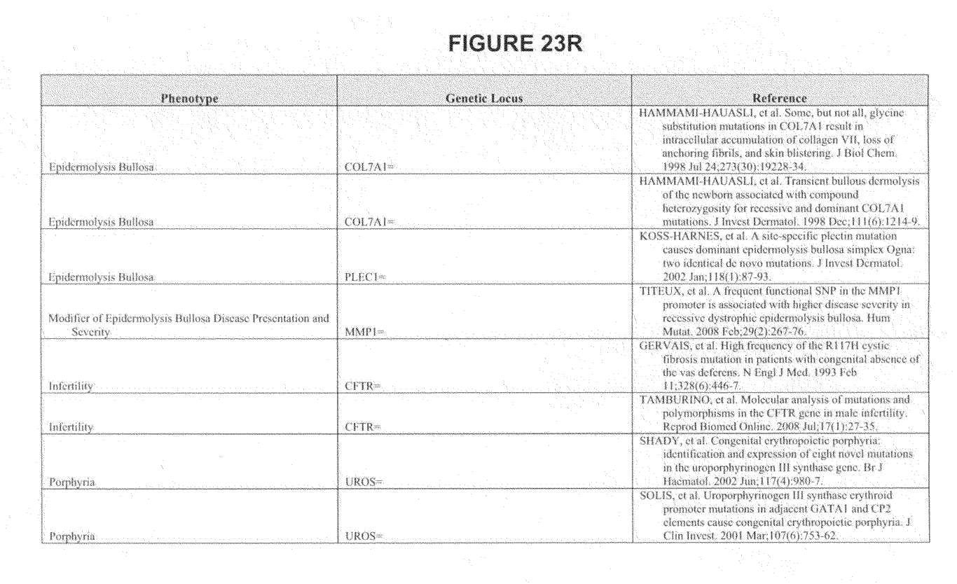

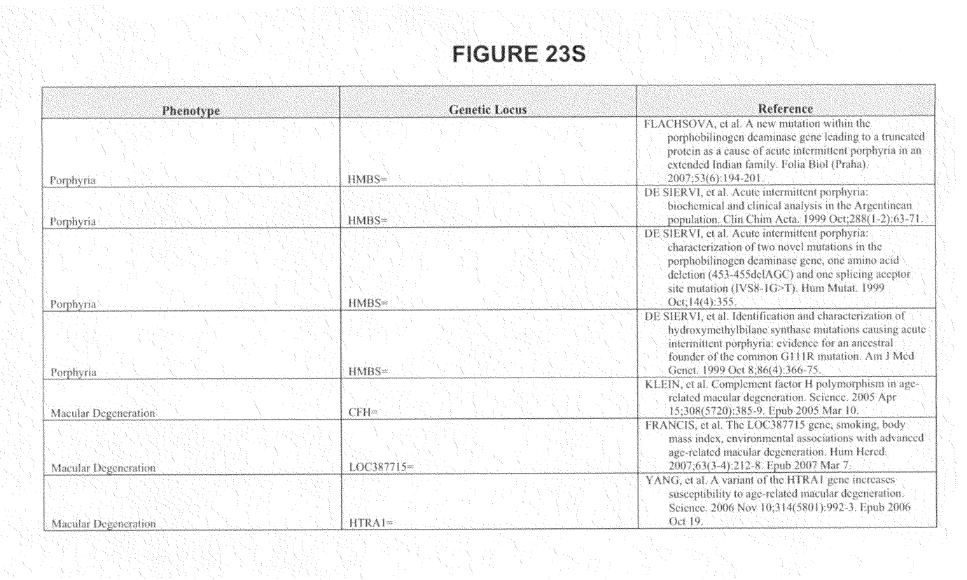

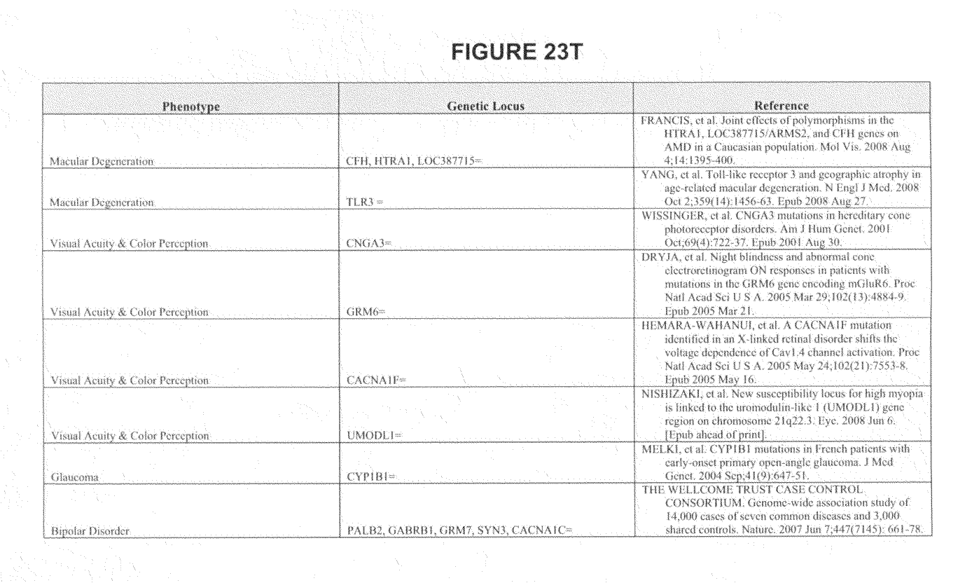

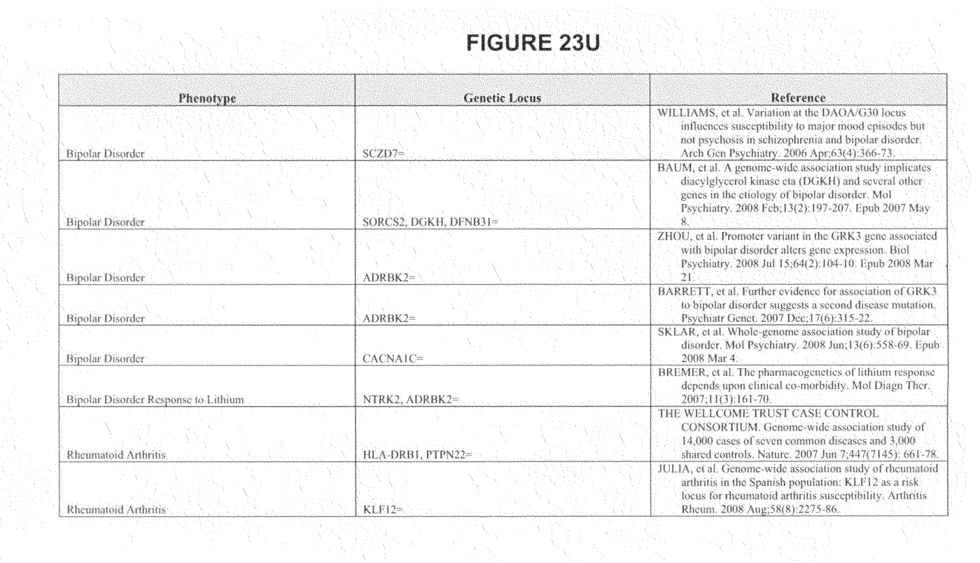

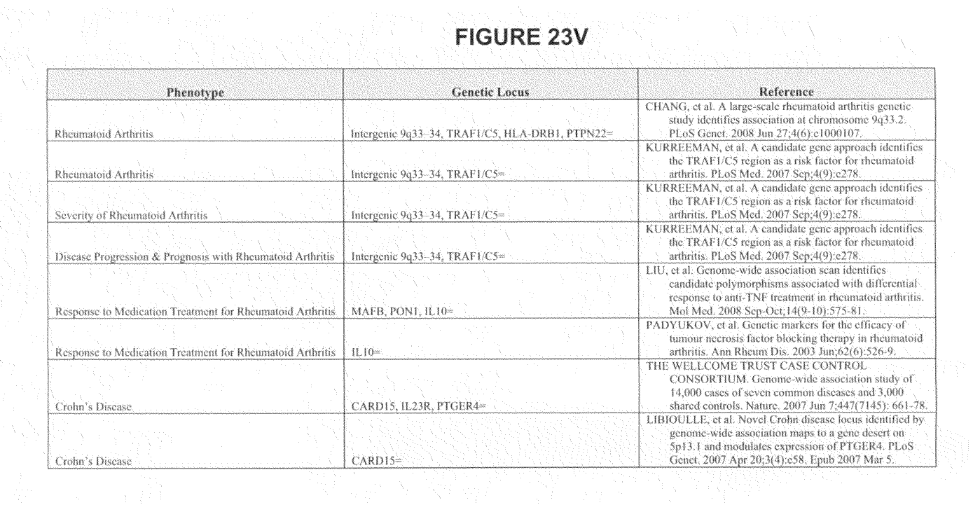

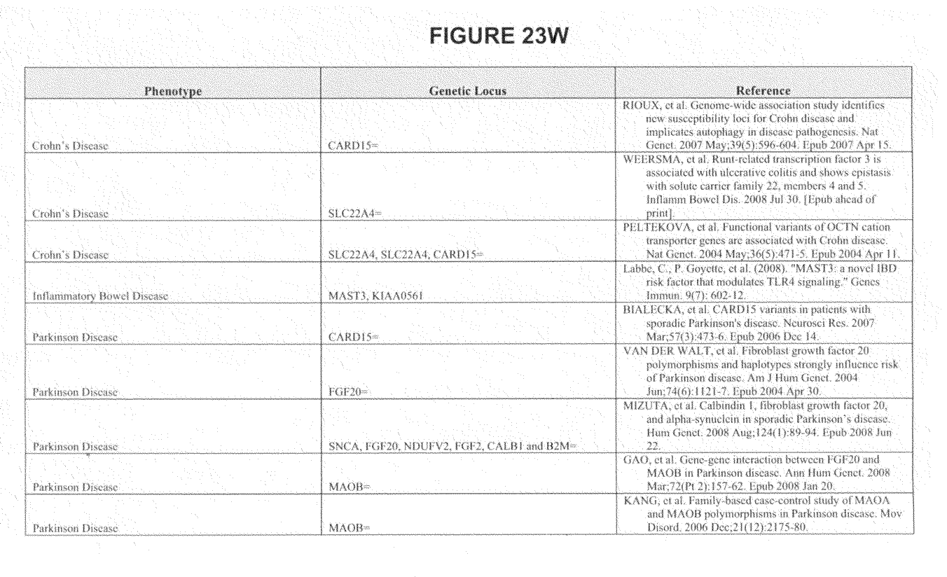

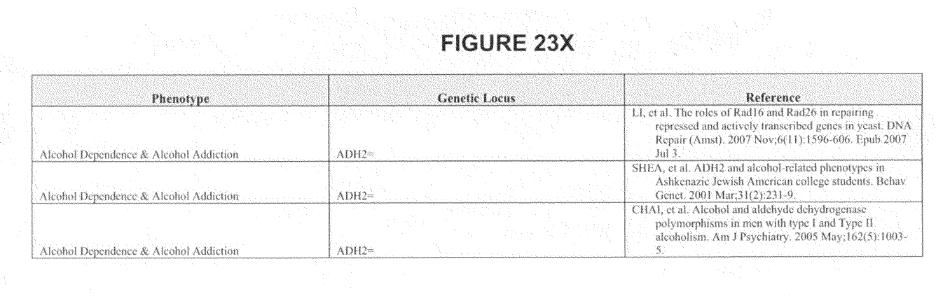

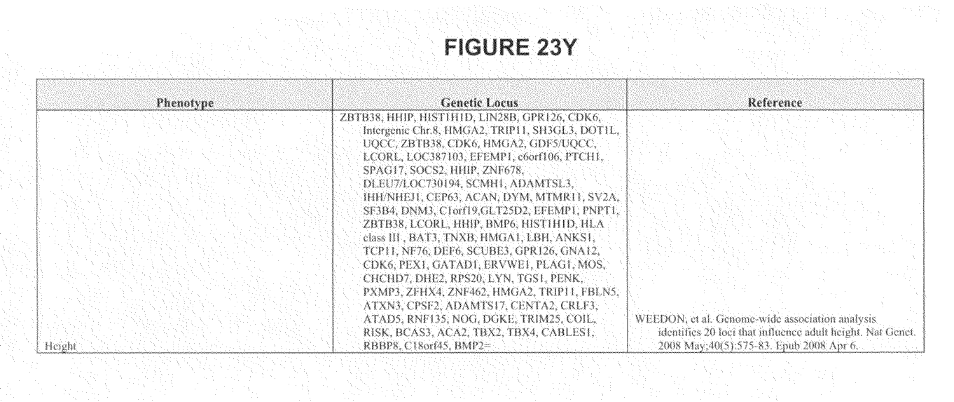

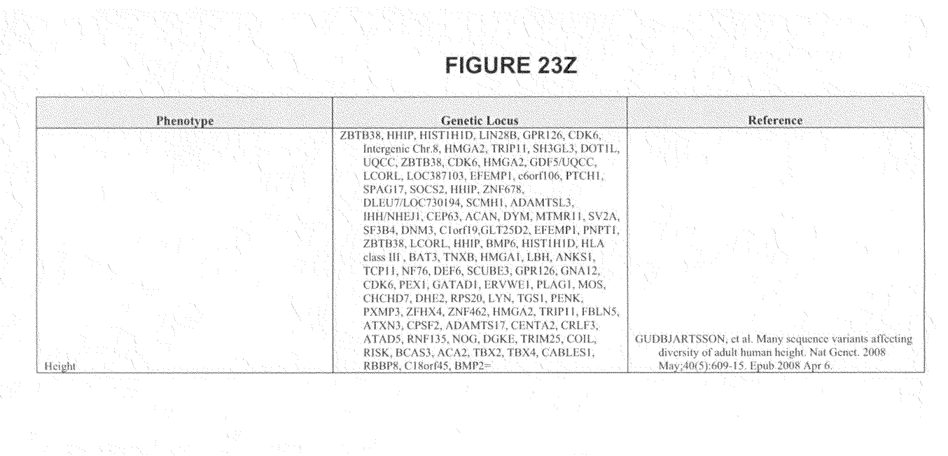

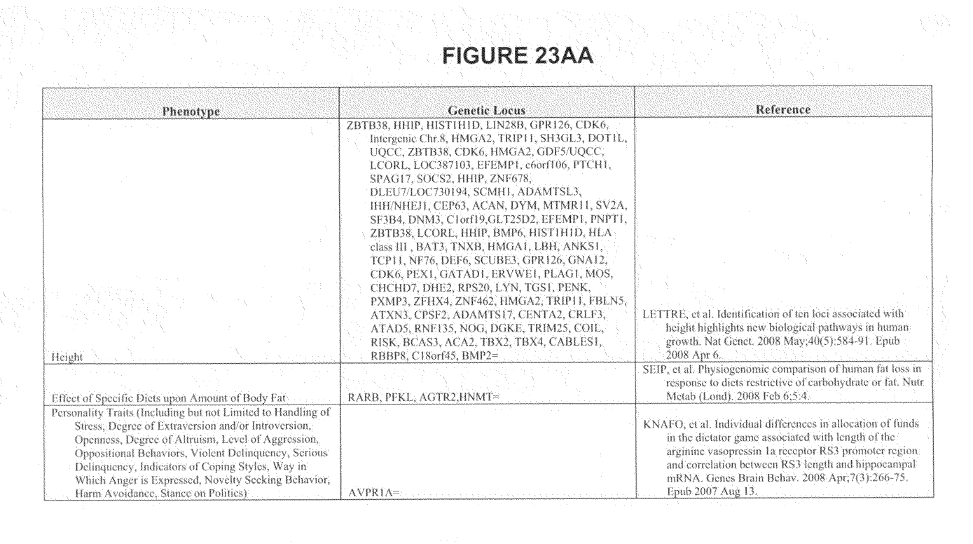

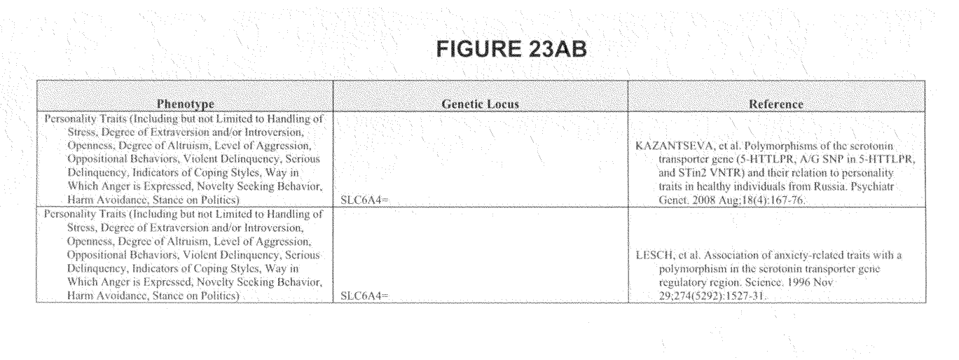

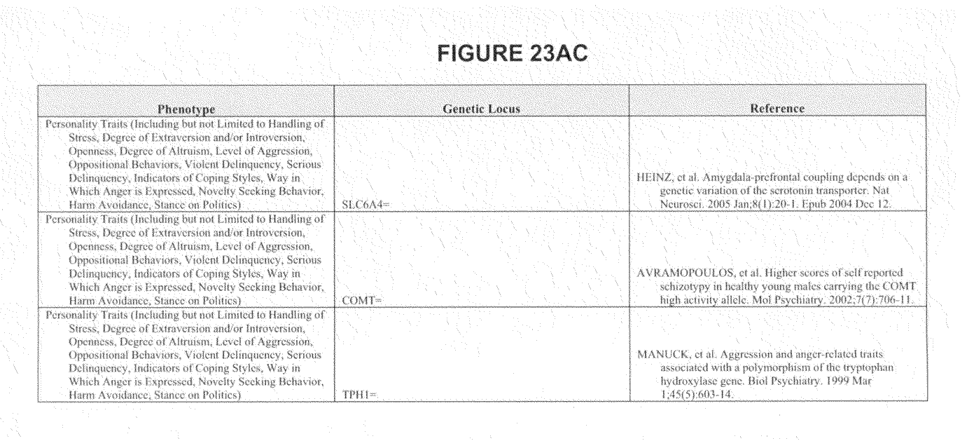

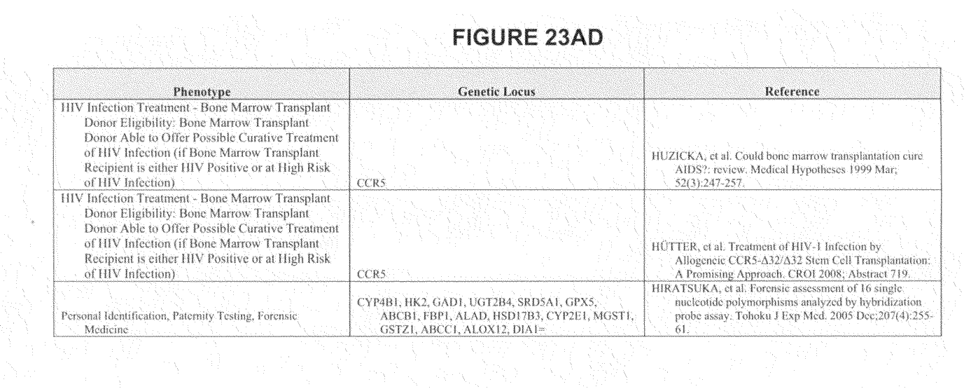

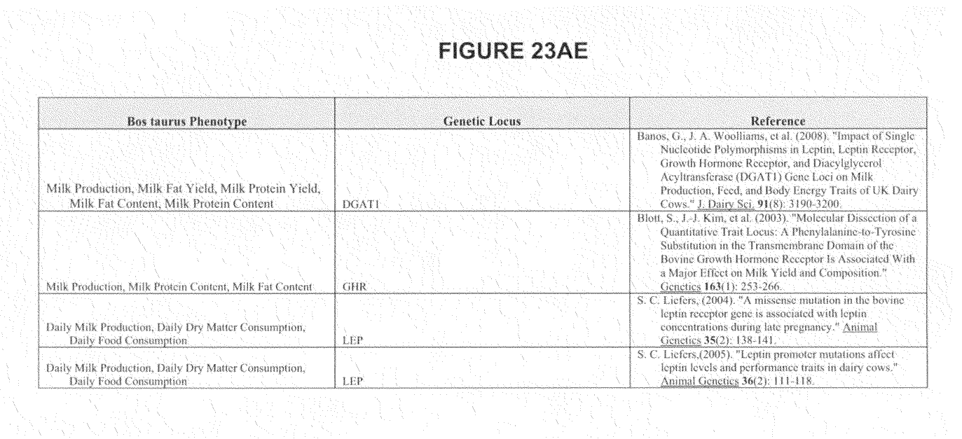

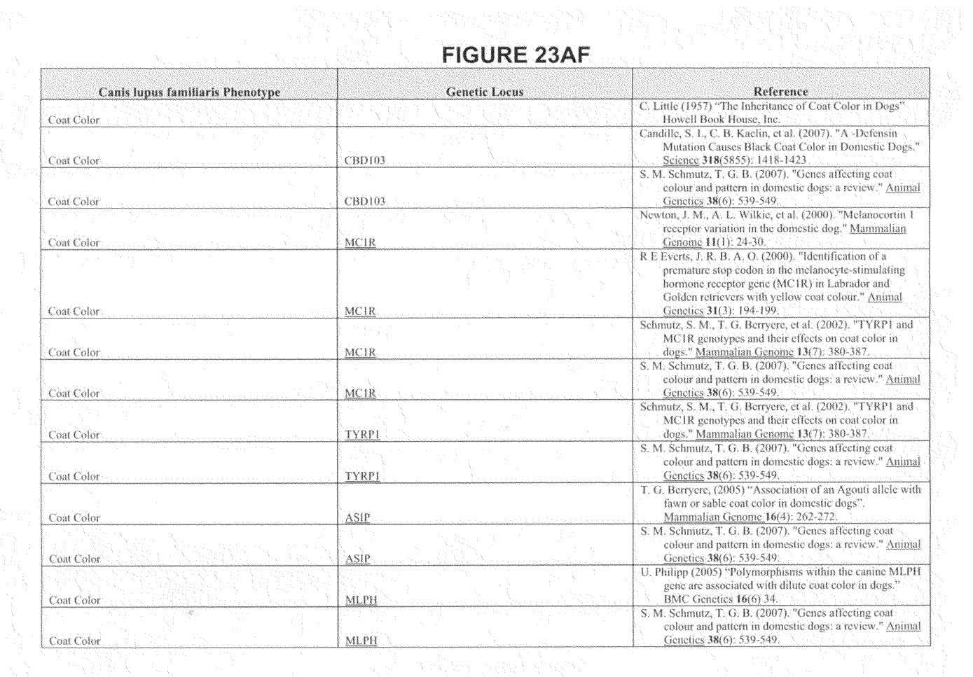

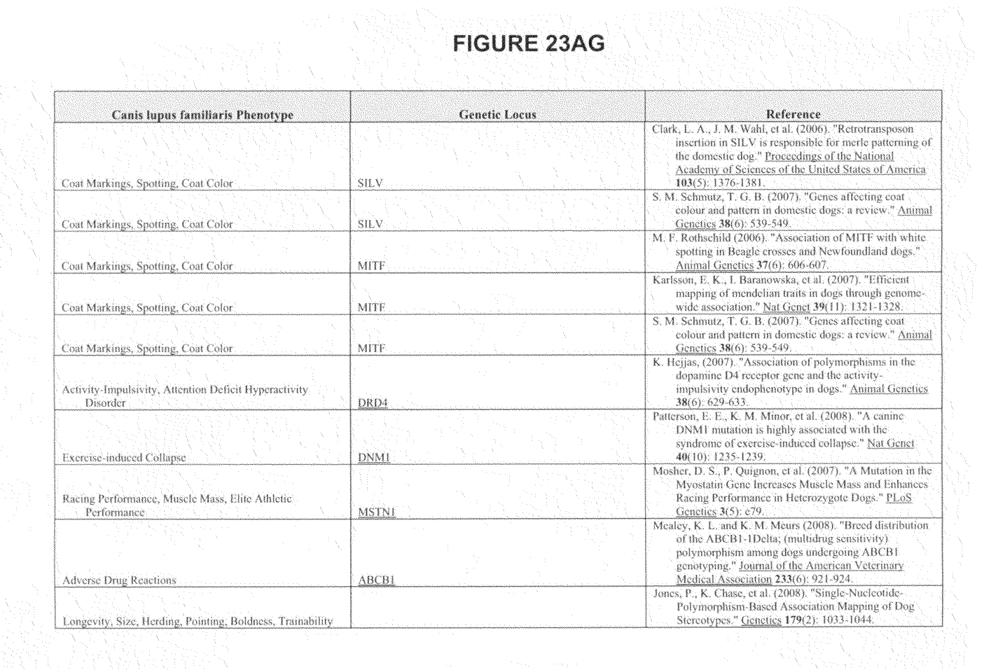

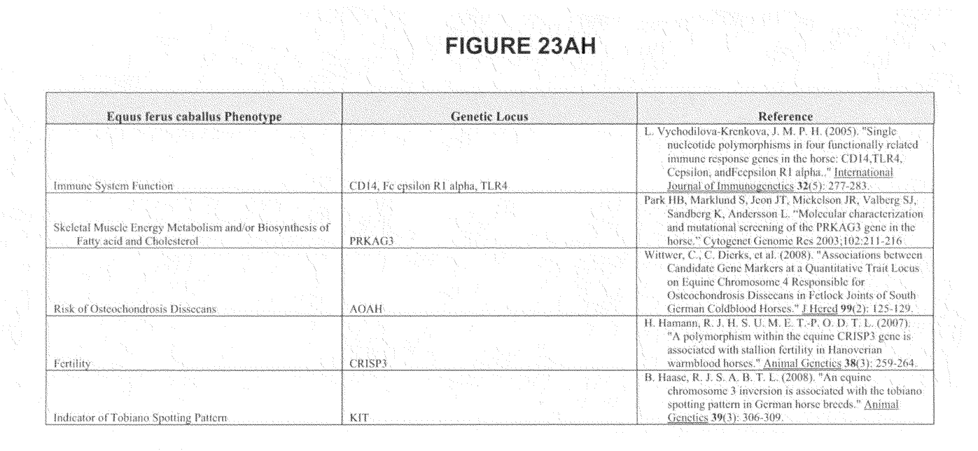

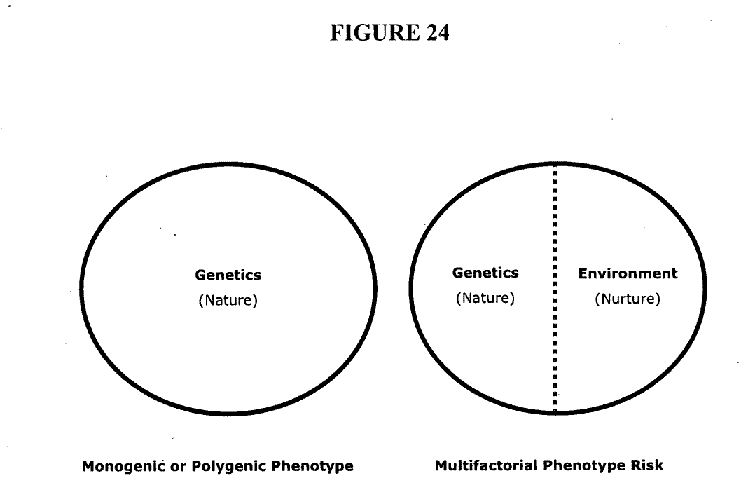

The risk for a phenotype (e.g., specific disease, disorder, characteristic, trait or condition), including responses to drug treatments, such as efficacy of a drug, may be represented by a score or action score. For example, a score or action score for a specific disease or trait can be determined by multiplying the phenotype's Clinical Significance Rating (CSR), Phenotype Impact Rating (PIR) and Notice Me Factor (NMF). In other embodiments, an Action Score (AS) may be determined by using a subset of the aforementioned factors, additional factors, or a combination thereof, as further described below. Other scores or measures may also be determined (See for example, FIG. 6, Table 8, Table 9A-9B and Example 7).

The Generic Lifetime Risk (GLR) is the gender-specific or gender matched lifetime risk of a specific phenotype for a population and this can be obtained from published literature and various resources such as from the United States Department of Health and Human Services' Centers for Disease Control and Prevention (CDC) and National Institutes of Health (NIH). The GLR may also be age-matched and/or gender-matched for a population. The Cumulative Genetic Risk (CGR) is the individual's risk of a phenotype based on their genetic profile, containing one or more genetic variants associated with risk for that phenotype, and is determined by taking into account all relevant genetic variants associated with that phenotype. The Predictive Medicine Risk (PMR) is the individuals new lifetime risk for a phenotype based on the phenotype's GLR and the individual's CGR.

The PIR (also known as the DIR), or Phenotype Impact Rating, indicates the clinical severity of a phenotype. For example, the PIR ranges from −3 to +3, where −3 causes sudden death or debilitating phenotype, such as a disease, −2 indicates a serious phenotype, such as a disease, a phenotype, such as a disease or condition, that is difficult to cure, may cause death, or has significant negative life consequences, −1 indicates a phenotype, such as a disease or condition, that is usually manageable, 0 is a neutral phenotype, such as a condition or trait, +1 indicates a slightly positive phenotype, such as a condition or trait; +2 indicates that the phenotype is a helpful trait or protection against (lower risk of) a harmful phenotype, such as a condition, and +3 indicates a significant advantage or significant protection against a harmful phenotype, such as a condition.

The Genetic Variant-Phenotype Score (GVP score), or the Genetic Variant-Disease or condition Coefficient (GVDC), may be used as a measure or rating system for genetic variant-phenotype correlations, or the association or strength of association between a genetic variant's allele or genotype and a phenotype, such as a disease or condition. For example, a GVP score can be determined for a disease or trait, such as breast cancer, based on studies correlating a genetic variant, such as a SNP with a phenotype, such as a disease or condition.

As the association between polygenic and multifactorial genetic variants and their phenotypes, such as diseases, disorders or traits, is complex, there may exist different levels of replication, validation, substantiation and confirmation that a genetic variant is associated with a specific phenotype, such as a disease, disorder or trait. For example, research (e.g., a clinical study) as to the association between genetic variant A and disease X may either be preliminary or may be highly substantiated or validated through studies in different cohorts that replicate similar results. An individual may have different levels of associations between a genetic variant and a phenotype determined and reported. For example, an individual's genetic profile may be reported with different sections divided by the level of replication, substantiation, validation and confirmation (e.g., the level the associations have been replicated, substantiated, validated or confirmed). The report may have a first section that contains genetic variant-phenotype associations that are only highly replicated and substantiated while the second section contains phenotype information assessed from genetic variant-phenotype associations that are highly replicated and substantiated and also moderately replicated and substantiated, and so forth. For example, there may only be preliminary information about a genetic variant's association with toxicity for a medication used in kidney transplant recipients. A kidney transplant physician or researcher, such as a clinical trial researcher, may find this information useful in watching adverse reactions or in determining the starting dose of the medication even if the association is not substantiated by replicated studies.

Factors that can be used in a system for rating genetic variant alleles or genotypes and their correlations with one or more phenotypes may include, but are not be limited to, the aggregate number of people in the disease cohort(s), or cohort(s) exhibiting a certain condition or trait, across all studies for the population (such as a population with the same ethnicity, nationality, gender, age, lifestyle, habits, occupation, past medical history, suspected medical condition, surgical history, social history, family history, prior genetic testing or analysis results, prior laboratory results, medications currently taking, medications previously taking, medications that may be given in the future, or any combination thereof), the aggregate number of people in the control cohort(s) across all studies (such as for a population with the same ethnicity, nationality, gender, age, lifestyle, habits, occupation, past medical history, suspected medical condition, surgical history, social history, family history, prior genetic testing or analysis results, prior laboratory results, medications currently taking, medications previously taking, medications that may be given in the future, or any combination thereof), the aggregate number of total people in the studies (such as a population with the same ethnicity, nationality, gender, age, lifestyle, habits, occupation, past medical history, suspected medical condition, surgical history, social history, family history, prior genetic testing or analysis results, prior laboratory results, medications currently taking, medications previously taking, medications that may be given in the future, or any combination thereof), a rating of the journal(s) that publish the articles that the genetic variant-phenotype associations are from (such as an internal rating scale, the Impact Factor, the Immediacy Index, the Cited Half-life, or the Page Rank, such as discussed further below), the type of study (Genome Wide Association Study, Case-Controlled Study, Meta-Analysis Study, Prospective Study, Retrospective Study, etc.), the institution that conducted the study (Wellcome Trust, Coriell Institute, Kaiser Permanente, deCODE, multinational collaborations, Mount Sinai Medical Center, Stanford University Medical Center, Harvard Medical School, Massachusetts General Hospital, University of California San Francisco Medical Center, Cedars-Sinai Medical Center, etc.), the place the study was conducted (for example, United States, United Kingdom, Netherlands, Iceland, Norway, France, Italy, Japan, Australia, Spain, Russia, China, multicontinent, etc.), or the year the study was conducted.

The GVP Score, also known as the GVDC, is an example of a system used for rating a genetic variant-phenotype correlation (see for example, FIG. 7). It may be the only system used or combined with other systems, as further described below. Thus in the embodiments described herein, other rating systems (such as those described below) may be used instead of the GVP score, or in combination with the GVP score. The GVP score may be population specific or it may not be population specific. In some embodiments, the GVP score is designated as 0 when there are 2 or more contradictory studies pertaining to the genetic variant and the phenotype, such as a disease or condition or, if there are three or more studies pertaining to the same genetic variant-phenotype association in the same population then the score is a 0 when there is contradiction in one or more of the top three studies (including meta-analysis studies) with the highest power (the largest number of individuals in the study cohort); 0.25 for a single study with single disease cohort study population containing under 250 individuals; 0.50 for a single study with a single disease cohort study population containing over 250 individuals; 0.75 for a single study with two or more disease cohort study populations (each disease cohort population can be the same or different ethnicities or gender), with each containing under 250 individuals; 1 for a single study with two or more disease cohort study populations (each disease cohort population can be the same or different ethnicities or gender), each containing 250-999 individuals and each giving similar results; 1.25 for a single study with two or more disease cohort study populations (each disease cohort population can be the same or different ethnicities or gender), each containing over 1,000 individuals and each giving similar results; 1.50 for one primary study and one replication study, each with similar findings (same phenotype, such as disease, association and same direction of risk); 1.75 for one primary study with two or more replication studies, each with similar findings (same phenotype, such as disease, association and same direction of risk); 2 for two or more genome wide association studies (GWAS) with similar results; and 2 for a monogenic disorder where the genetic variant is found to segregate with the phenotype, such as a disease, or the genetic variant is found within a gene that has previously been associated with the phenotype, such as a disease, or likely to be associated with the phenotype, such as a disease, or laboratory (such as in vitro studies, in vivo studies, biochemical studies, molecular biology studies, computational models or studies, bioinformatic studies, phylogenetic studies, etc.) evidence that the genetic variant causes a change in the characteristics of its genetic sequence, a nearby genetic sequence, the protein produced from that gene, or a protein or molecule (such as microRNA) that interacts with the genetic sequence containing, or located near, the genetic variant. The designation of “contradictory studies” occurs when one study finds a statistically significant association between a genetic variant and a phenotype while another study finds a statistically non-significant association between that same genetic variant's allele or genotype and the same phenotype or a genetic variant's allele in tight linkage disequilibrium with the original genetic variant's allele and the same phenotype. Contradictory studies may also exist when a study finds an opposite direction of association between the same allele or genotype of the same genetic variant and the same phenotype, such as if one study of a genetic variant finds increased risk of a phenotype while another study of the same genetic variant's allele or genotype or a genetic variant's allele in tight linkage disequilibrium with the original genetic variant's allele finds decreased risk of the same phenotype. However, studies that find different degrees of association (that are in the same direction) are not considered contradictory, such as, for example, if one study finds that a genetic variant's allele or genotype is associated with an increased risk of the phenotype with an odds ratio=1.25 and a second study also finds an increased risk of the phenotype with an odds ratio=1.65. This is considered confirmatory, not contradictory.

For example, if both study X and study Y were both case-controlled studies, both studied the same genetic variant or two genetic variants that are in linkage disequilibrium with each other, both looked at 1,500 and 5,000 African Americans in the study (disease) cohort, respectively, and both reported an increased risk of disease Z, then if the rating system as described above is utilized, the GVP score is 1.50 since there were two studies with similar results. The rating system being used, such as the GVP score, can be entered into the database along with the genetic variant (for example, the rs number from the dbSNP database, the chromosome that contains the genetic variant, the location of the genetic variant within a specific gene or chromosome such as its amino acid number and amino acid change (eg. Asp changed to Val at position 325) or the exact chromosome and chromosomal position as per Ensembl's coordinate numbering system, the specific sequence with 4, 5, 6, 8, 10, 15, 20, 30, 40, 50 bp or more of sequence information surrounding and including the genetic variant included in the database or a linked database described herein, or some other type of identification that allows the exact position of the genetic variant to be discerned within the genome), and the risk information (such as the odds ratio or the relative risk or the hazard ratio or the absolute risk or the cumulative risk or some other value, either quantitative or qualitative), and the allele or genotype associated with the phenotype, as well as the specific population that this information is applicable to (such as ethnicity, nationality, gender, age, body mass index, lifestyle, habits, occupation, past medical history, suspected medical condition, surgical history, social history, family history, prior genetic testing or analysis results, prior laboratory results, medications currently taking, medications previously taking, medications that may be given in the future, or any combination thereof).

The rating system may also include Replication Status rating, such as whether an association between a genetic variant with a phenotype has been replicated in two or more studies (Yes), has not been replicated yet (No), has been replicated only in two or more disease cohorts within the same study (Within), or has failed replication in comparing two or more studies (Failed). The rating scales, including the Replication Status rating, are applicable to all types of genetic variant-phenotype associations, including multigenic, multifactorial, and monogenic. In some cases, such as for example for monogenic phenotypes, reported results can be considered very reliable even without replication of the results. Accordingly, in some embodiments of the present invention, a Replication Status of “Mono” can be assigned for monogenic phenotypes. In some cases, the replication status of “Mono” can be assigned for reported monogenic phenotypes that have not been replicated, indicating that they are nevertheless more reliable than non-replicated polygenic or multifactorial phenotypes, and a replication status of “Yes” or “Failed” can be assigned for monogenic phenotypes that have been replicated. In other cases, all monogenic phenotypes may be given a replication status of “Mono.” The Replication Status rating can be in addition to the GVP score or in-place of the GVP score. If there are three of more studies, where one or more contains data that is contradictory to the other studies (such as if two studies find a statistically significant association between a genetic variant's allele or genotype and a phenotype but a third does not) for the same population, then the studies with the highest power (number of people in the study cohort) are considered most relevant. If the top three studies (including meta-analysis studies) with the highest power (the number of individuals in the study cohort) confirm the same genetic variant's genotype-phenotype association (or if they confirm the phenotype association with two or more genetic variants' that are in linkage disequilibrium with each other), then the genetic variant's genotype-phenotype association is assigned a “Yes”. If the top three studies with the highest power have contradictory results for the genetic variant's genotype-phenotype association, then the association is assigned a “Failed”. As new studies are conducted and data released, this designation may change as a new study may have a high enough power to put it in the top three and therefore its results will be considered in the analysis and designation of “Yes”, “No”, or “Failed”.

This rating system may be utilized to make the genetic analysis and final genetic report for an individual's genomic profile either more or less substantiated, or to include the genetic variant in some panels (further described below) or some genetic analysis (including, but not limited to, one or more of the following: analysis to calculate the risk such as predictive medicine risk, the calculation of organ risk, calculation of genetic health, and inclusion or exclusion of the genetic variant and its associated data within the genetic report) and not others. The genetic report may contain genotype information, genotype-phenotype associations, preventive medicine recommendations or interventions.

For example, an individual or their health care provider or manager or other third party, may request, order, obtain, or have an individual's genomic profile that provides only genetic variants associated with phenotypes that have a specific threshold value for one or more of the rating systems utilized. For example, the threshold value can be a specific value, such as above or below a specific value or it can be a range. For example, the threshold value for the GVP score can be above 1, below 1, or a range of values, such as any value between 0.25-1.25, any value not between 0.25-1.25. Alternatively, the threshold value can be a single numerical value such as 2. The analytical system is fully configurable so that any combination of threshold values for one or more rating systems can be combined in order to filter the analysis and results according to those selected thresholds. For example, FIG. 6B shows a genetic data analysis with a threshold GVP Score equal to or greater than 1.5, FIG. 6C which shows a genetic data analysis with a threshold of only monogenic phenotypes, and FIG. 6D which shows the threshold as being either Replicated associations or Monogenic phenotypes.

Highly substantiated associations, such that only those with a GVP score (rating) of 1.50 or above, or an even higher threshold of 1.75 or above, may be reported or determined, and all other genetic variants excluded. Alternatively, all possible associations and all genetic variants found associated with a specific disease or a panel or a organ system can be included in the analysis, but those with contradictory studies are omitted, therefore the GVP score threshold is 0.25 or above. Thus, all genetic variants with a GVP score (also known as GVDC) of 0.25 or above that are associated with a specific phenotype, such as a disease, trait, condition or process, or that is included in a panel or an organ system, can or will be utilized in the analysis of predisposition and risk and may also be utilized to determine the Predictive Medicine Risk, organ score, genetic health score, or one or more of the above, and included within the genetic report.

The threshold value selected may be selected by the individual's whose genomic profile is being used, a health care manager of the individual, a medical professional, a medical entity such as a hospital, a laboratory director, or another third party. Alternatively, the threshold value may be determined by the party or entity, such as a company or laboratory generating the genetic data, as that party or entity may have one or more preset threshold values. Alternatively, the threshold values may be determined by an individual in consultation with the party or entity generating the genetic data, their health care manager or provider, or another third party.

The report for an individual's genomic profile may also contain all known associations, but the associations are divided into sections by the level of association. For example, the report may have section 1 that contains only genetic variant-phenotype (disease/trait/condition) score associations with a cut off of 1.75 or above, section 2 may contain genetic variant-phenotype (disease/trait/condition) score associations with a cut off of 1.5, section 3 may have a cut-off of 0.75-1.25, and section 4 may have a cut-off of 0.25-0.50. Furthermore, the reported GVP score may be changed at a later date. For example, an initial report for only highly substantiated associates can be generated for an individual, and a later report with all associations (i.e. a lower GVP score threshold value) is provided in a subsequent report. This rating system may also be updated, for example, by incorporation of new journal articles and data on an on-going basis. For instance, a genetic variant associated with a phenotype is assigned a GVP of 0.25 and another study is discovered or published that shows the same phenotype associated with the same genetic variant in the same population and the study and results are statistically significant. The GVP is then raised to 1.5. As a result, new reports can be generated based on incorporation of new journal articles and new studies and as a result new GVP score values for genetic-variant-phenotype associations. The new or updated reports may be produced from the initial data obtained from analyzing the genetic variants of an individual, the initial genetic sample obtained from an individual, or from a new sample. The new or updated reports may be provided for an additional fee.

In some embodiments, two or more different versions of the genetic report may be created utilizing this rating system. For example, an individual may order a panel through his or her cardiologist. The report produced for the cardiologist may only contain information on genetic variants and their phenotypes that have GVP score (coefficients) of 1.5 or greater while the report produced for the individual may contain information on genetic variants and their phenotypes with a GVP score of 0.75 or greater. In other cases, the report produced for the cardiologist may only contain information on genetic variants and their phenotypes that have a GVP score of 0.75 or greater, while the report produced for the individual may contain information on genetic variants and their phenotypes with a GVP score of 0.75 or greater. As another example, a physician ordering the genetic testing and/or analysis may request a GVP score of 1.5 or greater but a medical researcher who is also working with the same patient may request a GVP score of 0.25 or greater. As another example, for a patient with an illness of unknown etiology, a physician may order the genetic testing and/or analysis with two different GVP scores, such that one report or one section of the report contains analysis and information pertaining to only GVP scores of 1.75 or greater while the second report or another section of the same report contains GVP scores of 0.5 or greater, thereby allowing the physician to assess not only his or her patient's risk or predisposition or affected status or carrier status for the phenotypes contained in the genetic testing and/or analysis panel ordered based on replicated research but to also receive information on genetic variants and phenotypes that are not replicated yet but may still provide useful information for the physician or the patient or both. Genetic analysis or genetic reports or both ordered with more than one GVP score threshold value may be provided for an additional fee.

Furthermore, in some embodiments, a specific genetic variant may have more than one GVP score, such as if it is associated with more than one phenotype. For example, the same genetic variant's genotype may be associated with increased risk for prostate cancer as well as a decreased risk for diabetes mellitus, type II. The GVP score for genotype-phenotype association with prostate cancer may be 1.5 while the GVP score for the genotype-phenotype association with diabetes mellitus, type II, may be 2. If the cut-off value for the GVP score was set at 1.75 and above, then this genetic variant and its data for diabetes mellitus, type II would be utilized in the analysis for diabetes mellitus, type II, in order to determine risk for diabetes mellitus, type II, including risk analysis, PMR, AS, organ score, or genetic health score, but this genetic variant would not be utilized in the analysis for prostate cancer as the GVP score threshold value is above the GVP score for the prostate cancer phenotype for that genetic variant.

In some aspects of the present invention, the aggregate number of people with the phenotype, such as a disease or condition, cohort(s) (also referred to as the disease cohort(s) or the study cohort(s)) such as described above for the GVP score, may be the sole factor or in combination with other systems described herein, for rating a genetic variant or genotypes and their correlations with one or more phenotypes. The rating system for the GVP score can include information pertaining to the number of studies (such as journal articles) that have shown an association between that exact genetic variant (or a genetic variant in linkage disequilibrium with that genetic variant, such as an r²>0.3), as well as whether or not one or more of those studies was a Genome-Wide Association Study.

Other rating systems may be used instead of the GVP score, or in combination with the GVP score in evaluating the genetic variant-phenotype association. For example, all journal articles pertaining to genetic variants and their allele or genotype-phenotype association may be included automatically for computing a GVP score. Alternatively only specific journal articles, such as those decided to be added to the database or added to the genetic analysis or both, may be used. For example, the journal articles or publications may be analyzed before incorporating and storing both the article and its corresponding data and information within a database.

A journal article relating to one or more genetic variants and their association with any phenotype may be read and analyzed, by a human or automated to be fully accomplished or partially accomplished by a computer or other information technology system or software. A scaling system (such as numbers, letters, colors, symbols or combinations thereof) is then applied to the journal article based on numerous factors of that journal article. The factors of the journal article that are taken into account may contain the number of people in the disease (study) cohort, the number of people in the control cohort, the total number of people in the study, the institution that conducted the study, the place the study was conducted (such as state or country or region or continent), a rating for the journal itself (ratings may include, but not be limited to, an internal rating or the Impact-Factor of the journal, such as the system created by Eugene Garfield at this Institute for Scientific Information, the Immediacy Index of the journal (such as published in the Journal Citation Reports), the Cited Half-life of the journal, the Page Rank of the journal, or any other measure), the year the study was published, the type of study that was conducted (for example, Genome Wide Association Study (GWAS), Case-Control Study, Prospective Study, Retrospective Study, Meta-Analysis Study) the name of the journal, the name or reputation of any or all of the authors involved in the study, or any and all combinations of the factors thereof, such as shown in Table 5.

TABLE 5
Journal Article Factors

| Journal Article Factors | Rating Scale |
|---|---|
| Number of people in | <250 = 1 |
| Disease (Study) Cohort(s) | 250-999 = 2 |
| | 1000-2499 = 3 |
| | 2500-4999 = 4 |
| | 5000-9999 = 5 |
| | ≧10,000 = 6 |
| Number of people in | <250 = 1 |
| Control Cohort(s) | 250-999 = 2 |
| | 1000-2499 = 3 |
| | 2500-4999 = 4 |
| | 5000-9999 = 5 |
| | ≧10,000 = 6 |
| Total Number of People in | <250 = 1 |
| the study | 250-999 = 2 |
| | 1000-2499 = 3 |
| | 2500-4999 = 4 |
| | 5000-9999 = 5 |
| | ≧10,000 = 6 |
| Institution that Conducted | US News & World Report Ranking for |
| Study | Top Hospitals or Medical Institutions |
| | or Medical Schools |
| | >#50 = 1 |
| | 11-50 = 2 |
| | ≦10 = 3 |
| | Outside of US & UK = 1 |
| | Wellcome Trust = 3 |
| | DeCode = 3 |
| | Broad Institute = 3 |
| | Multinational Study = 3 |
| Place Study was | Eastern Europe = 1 |
| Conducted | Asia (Except Japan and Singapore) & |
| | Latin America & Middle East |
| | (Except Israel) = 2 |
| | Japan & Singapore & Israel = 3 |
| | Western Europe (Except UK) & Australia |
| | & New Zealand = 4 |
| | United States & United Kingdom = 5 |
| Impact-Factor of Journal | <10 = 1 |
| | 11-25 = 2 |
| | 26-35 = 3 |
| | >35 = 4 |
| Immediacy Index of | <3 = 1 |
| Journal | 3-4 = 2 |
| | >5 = 3 |
| Cited Half-Life of Journal | <2 = 1 |
| | 2-3 = 2 |
| | >3 = 3 |
| Page Rank of Journal | <3 = 1 |
| | 3-10 = 2 |
| | >10 = 3 |
| Year Study was Published | <1980 = 1 |
| | 1980-1989 = 2 |
| | 1990-1994 = 3 |
| | 1995-1999 = 4 |
| | 2000-2003 = 5 |
| | 2004-2006 = 6 |
| | >2006 = 7 |
| Type of Study | Retrospective or Prospective = 1 |
| | Case-controlled = 2 |
| | Meta-Analysis = 3 |
| | GWAS = 3 |
| Name of Journal | Nature, Nature Genetics, Science, New |
| | England Journal of Medicine, Proceedings |
| | of the National Academy of Sciences, Cell, |
| | The Lancet, Journal of the American |
| | Medical Association, American Journal |
| | of Human Genetics = 3 |
| | All others = 1 |
| Name/Reputation of | Unknown = 1 |
| Author(s) | One or more prior articles on same gene |
| | or gene family or disease = 2 |

The rating scale categories for a journal article, such as shown in Table 5, may be used individually, or in various combinations, in determining a ranking system for the journal article, or in identifying a threshold value (such as described for GVP score herein), for including or excluding, the information in determining predisposition values, risk values, a genotype, a phenotype, or any such association between a genetic variant and a phenotype, such as a disease, trait, condition, or process. The rating or value given to a journal article may indicate that the journal article should be read or not read, that the journal article or its data should be included in the database or not included in the database, that the journal article or its data should be included in the genetic analysis of a person or not included in the genetic analysis, or that the journal article or its data should be included in the genetic report or not included in the genetic report.

For example, if the factors chosen to be analyzed include the number of people in disease cohort and impact factor of the journal, then the threshold may be: below 5 do not include in database, 5-6 include in database but not in genetic analysis, and 7 or greater to include in database and include in genetic analysis. For a journal article that contains 1,500 people in the disease cohort and is published in a journal with an impact factor of 36.98, the rating scale value would be 3+4=7 and therefore the journal article, its data, or both are included in both the database and the genetic analysis. For a journal article that contains 5,000 people in the disease cohort and is published in a journal with an impact factor of 6, then the rating scale value would be 5+1=6 and therefore the journal article, its data, or both is included in the database but not in the genetic analysis. For a journal article that contains 125 people in the disease cohort and its journal has an impact factor of 8, then the rating scale value would be 1+1=0.2 and the journal article, its data, or both may not be analyzed and may not be included in the database or the genetic analysis.

Another rating system that may be used in combination with other systems described herein, or alone, is a rating system that determines whether or not the genetic variant's genotype-phenotype association for a specific genetic variant existing anywhere in the genome has been replicated, called the Replication Status. Replication can either mean two or more studies have shown the same direction (increased risk or decreased risk) for that genetic variant in the same or similar populations. An alternative system requires that at least 3 or more, 4 or more, 5 or more, etc. studies have arrived at similar results as stated above. Status of replication for each genetic variant can be designated either a simple Yes/No. Alternatively, status of replication can be a scale, such as Definitively Replicated, Moderately Replicated, Not Replicated Yet, or Failed Replication (if there are contradictory studies, such as a study that one or more studies that meet the threshold for the journal article factor(s) have shown no statistically significant genotype-phenotype association with that specific genetic variant or a genetic variant in linkage disequilibrium with that genetic variant). If a single study contains two or more separate disease cohorts and the genetic variant-phenotype association is similar in each cohort, then a separate rating of “Within” may be applied to the Replication Status for that genetic variant-phenotype association. Monogenic phenotypes can be also be represented according to replication status, being assigned a replication status of “Mono” if the genetic variant was shown to segregate with the phenotype, if it occurs in a gene previously implicated with the phenotype, if it occurs in a gene suspected of being implicated with the phenotype, or if biochemical, molecular, phylogenetic, computational, or bioinformatic analysis shows that the genetic variant is most likely deleterious or harmful or likely to be associated with a disease or phenotype. If there are three of more studies, where one or more contains data that is contradictory to the other studies (such as if two studies find a statistically significant association between a genetic variant's allele or genotype and a phenotype but a third does not) for the same population, then the studies with the highest power (number of people in the study cohort) are considered most relevant, as described herein.

This rating system may be utilized as described with the Replication Status, the GVP score or journal ranking system, in genetic analysis and generating genomic profiles and the genetic report by having more or less substantiated genetic variant-phenotype associations included or to include the genetic variant in some panels or genetic analysis (including one or more of the following: analysis to calculate the risk, the calculation of organ risk, calculation of genetic health, calculation of Predictive Medicine Risk, calculation of Notice Me Factor, calculation of action score, calculation of cumulative action score, and inclusion of the genetic variant and its data in the genetic report) and not others. For example, only replicated genetic variants may be included in the analysis of an individual's genomic information. If so, only the genetic variants that are designated as replicated (i.e. a Replication Status of “Yes”) within the database, such as the Predictive Medicine Database, or a linked database may be included in the analysis and in the genetic report. Alternatively, the person who orders the genetic test and/or analysis may want to know all possible associations and to have all genetic variants found associated with a specific disease or a panel or a organ system regardless of replication status and therefore both genetic variants that are designated as replicated and those that are designated as not replicated may be included in the analysis. All genetic variants with a chosen Replication Rating (whether it be a Yes/No/Within/Failed/Mono designation or a scale as exemplified previously) can be utilized in the analysis of predisposition and risk and may also be utilized in determining the Predictive Medicine Risk, Notice Me Factor, Action Score, Cumulative Action Score, organ score or genetic health score.

Other systems for ranking, and that may be used for selection by an individual or their health care professional, manager or provider for analysis or inclusion in a genetic analysis, a genomic profile, or a genetic report include the Genetic Variant-Phenotype Triage (GVP Triage, see for example, FIG. 8), also known as the GVP-Clinical Significance Rating (GVP-CSR, or CSR). A GVP Triage can be ranked numerically, where 0 would indicate no clinical use, 1 would indicate limited clinical significance, value, or use, 2 would indicate moderate clinical significance, 3 would indicate very useful in a clinical setting, where a medical professional would likely find the result valuable, and 4 would indicate extreme clinical significance, such as a life-threatening condition. The GVP Triage may be used also to determine whether genetic variants are included or excluded in genetic analysis or a report of the analysis. For example, genetic variants that have a GVP Triage of 2 or higher can be selected to be the only ones included in the analysis or report or both for an individual's genomic profile. Thus, similar to the aforementioned rating systems, GVP Triage values may serve as threshold values.

Each phenotype can have a separate GVP Triage rating assigned to it (for example, assigned by a licensed physician) for an increased risk of that phenotype and for a decreased risk of that phenotype. For monogenic phenotypes, each phenotype has a separate GVP Triage ratings assigned to it for the carrier state and for the affected state. The designation of carrier or affected is based on whether or not the genetic variant(s) associated with that phenotype are recessive or dominant in terms of Mendelian inheritance. For codominance, both alleles are considered dominant and the heterozygous genotype or diplotype may be associated with its own phenotype (such as Blood Type AB for the ABO blood group system in Homo sapiens) and for incomplete dominance, the heterozygous genotype may be associated with its own phenotype (such as with the Merle coat color trait in Canis lupus familiaris or with Sickle Cell Trait in Homo sapiens). As an example, for the hair color phenotype, the GVP Triage rating is “0” because hair color does not have clinical significance. However, for Long QT Syndrome, which can cause sudden death due to cardiac arrhythmias, the Long QT Syndrome phenotype is assigned a GVP Triage of “4” if the person is most likely affected with the syndrome because this information most likely requires immediate attention by a healthcare professional. Alternatively, if the person is a carrier of a genetic variant associated with Long QT Syndrome but is not affected by the syndrome, then this has less clinical significance and is assigned a rating of “2” because it is moderately useful (a healthcare professional may find this information useful in terms of educating their patient about the risk their children or future children may have in regards to Long QT Syndrome and also in educating their patient that a relative may carry or be affected by this syndrome and therefore may want to undergo genetic testing and/or analysis and health care professional consultation as well). The GVP Triage rating can occur at the genetic variant-phenotype level, so there is a GVP Triage rating (number) assigned to each genetic variant-phenotype association, meaning that there is at least one GVP Triage number assigned to each genetic variant.

The rating systems described herein may also be applied not to specific genetic variants but instead at the phenotype level, such as a disease, condition, or trait level. When this occurs, the rating system is no longer called GVP Triage but instead is called Clinical Significance Rating (CSR). The CSR is discussed below.

The Genetic Variant-Phenotype Rank (GVP Rank), also referred to as the SNP Ranking system, may be used to discern between genetic variants that are in linkage disequilibrium with each other (usually located within the same locus or within nearby loci) and that have been found to be, or can assumed to be, associated with the same signal or risk of the same phenotype. A GVP Rank may be provided for any two or more genetic variants and their alleles that are in linkage disequilibrium with each other and that are associated with the same or similar phenotype and the same direction of risk (either increased risk or decreased risk or no risk). The genetic variant, such as an SNP, with the most significant statistical association with the phenotype is indicated by a special designation, such as the number 1, and is therefore the highest ranking genetic variant, such as an SNP. The genetic variant, such as an SNP, with the second most statistically significant association with the phenotype is then assigned 2. The genetic variant, such as an SNP, with the third most significant statistical association with the phenotype is then assigned 3, and so forth.

For example, genetic variant A, B, and C may all be associated with a predisposition for early-onset heart attack, with genetic variant A having an odds ratio=1.40, genetic variant B having an odds ratio=1.35, and genetic variant C having an odds ratio=1.38. However, genetic variant A, B, and C are all in linkage disequilibrium with each other, with an r²=0.9 between A-B, A-C, and B-C as indicated by The International HapMap Project (HapMap). Published research indicates that genetic variant A is the most statistically significant genetic variant associated with early-onset heart attack out of A, B, and C and is therefore assigned the GVP Rank of 1, genetic variant B is the second most significantly associated with that phenotype and is assigned GVP Rank of 2, while genetic variant C is the third most significantly associated and is assigned GVP Rank of 3. The Cardiovascular Genetic Testing Panel may be chosen by the individual and genetic testing and/or analysis may find that the individual's genotypes for genetic variant A, B, and C are all associated with increased risk for early-onset heart attack. However, it may be inappropriate to include the risk values, such as odds ratios, for genetic variant A, B, and C in the analysis to determine the risk of early-onset heart attack as the risks of genetic variant A, B, and C may not be mutually independent (they may all be associated with the same signal that predisposes to that phenotype). Therefore, during the analysis process, genetic variant A, which has the highest GVP Rank (1) is the only genetic variant that is utilized within the analysis while the other genetic variants (B and C) are not further analyzed. Only genetic variant A's risk value information and data is therefore utilized to ascertain the risk GCR and PMR for early-onset heart attack. Genetic variant A's risk and data can be entered into an algorithm or computation that takes into account other genetic variants (not in linkage disequilibrium with genetic variant A) or genetic variant A may be analyzed on its own. If the genotype associated with early-onset myocardial infarction for genetic variant A is not detected, but genetic variants B and C are both detected, then the next highest GVP Rank genetic variant is B, so B is utilized in the analysis and in any calculations to ascertain risk for early-onset heart attack while C is not utilized in the calculations.

This methodology can also be applicable to haplotypes and diplotypes. For example, it may be found that haplotype X, that contains genetic variants A, B, and C, is also associated with early-onset heart attacks with an odds ratio=1.40 and is statistically more significant than A, B, or C alone. In this case, haplotype X is designated the GVP Rank of 1, genetic variant A is designated SNP Ranking of 2, genetic variant B is designated SNP Ranking of 3, and genetic variant C is designated SNP Ranking of 4. If the genotype results for the genetic test and/or analysis contain the alleles at genetic variants A, B, and C that constitutes haplotype X then only haplotype X, along with its data and risk information, is utilized in the further analysis and calculation of the individual's risk for early-onset heart attack because haplotype X has the highest GVP Rank (1). If the alleles of either genetic variant A, B, or C however, do not satisfy haplotype X, then haplotype X does not exist and therefore the methodology looks at the next highest GVP Rank, 2, which is genetic variant A, and so forth until either an allele or genotype associated with early-onset heart attack is found and that genetic variant's risk value is the only one (out of those that are in linkage disequilibrium with it and have assigned GVP Rankings) utilized in the analysis and calculation of risk. This methodology can also be applied to any genetic variants within the same haplotype block as opposed to linkage disequilibrium, or both haplotype block data and linkage disequilibrium data can be utilized together. This methodology can also be applied to any genetic variants that have been shown in published literature to be associated with the same signal for a phenotype or for a risk or predisposition to a phenotype.

The rating systems and analytical methodology described herein, such as the journal ranking, GVP score, GVP Triage, Replication Status and GVP Rank can all be utilized independently of each other, or in any combination of two or more, and can be included as categories in a database described herein. For example, the GVP score, GVP triage, and GVP Rank can be utilized together such that only diseases with a GVP triage of 2 or above and only specific genetic variants and their specific allele or genotype-phenotype association with a GVP score of 1.5 or above, and only genetic variants that are mutually independent of each other (are either not in linkage disequilibrium or are in loose linkage disequilibrium, such as an r²=0.1) may be included in the genetic testing, the genetic analysis and/or the Genetic Report.

The various rating systems may also be used to sort the results from genetic testing or analysis prior to any further analysis, processing, or the generation of the PMR, AS, CAS, or the genetic report. The various rating systems may also be used to choose and sort the genetic variants that will be tested for during the actual laboratory genetic testing and/or analysis process or the genetic variants that the laboratory will provide allele or genotypic information on. These rating systems offer significant control over what genetic variant-phenotype associations are included within the genetic testing, genetic analysis and genetic report and which are not, and allow for data to be pulled from a non-exclusionary Predictive Medicine Database that takes into account all known genetic variant-phenotype associations on the front end and allows for the filtering of these genetic variant-phenotype associations on the back end based on rating systems and thresholds as discussed.

Other rating systems may include the Phenotype's Clinical Significance Rating (CSR), which is a rating scale that assigns an integer (range is between 0 to 4) to each phenotype based on its clinical relevancy (for example, by a licensed physician), such as shown in Table 6. The rating scale allows for phenotypes with greater clinical relevancy to be able to be discerned efficiently from those with less clinical relevancy. The CSR is one of the components of the Action Score; because of this, one of the ways the Action Score is weighted is by clinical significance.

TABLE 6
Clinical Significance Rating (Csr)

| | Clinical | Clinical Significance Rating |
|---|---|---|
| Description | Significance | (CSR) |
| No Clinical Significance - most likely not of importance to a | None | 0 |
| healthcare professional. | | |
| May be carrier of a monogenic phenotype with | | |
| a CSR = 0-1 when affected. | | |
| Limited Clinical Significance - may be of limited importance to a | Limited | 1 |
| healthcare professional. Prevention and/or treatment options for the | | |
| phenotype may be severely limited, scarce, or highly experimental. | | |
| Not yet able to limit morbidity or mortality even when predisposition | | |
| is known prior to the manifestation of the phenotype. | | |
| May also be phenotype with marginal clinical importance, | | |
| such as Pityriasis capitis. | | |
| May be carrier of a monogenic phenotype with | | |
| a CSR = 2 when affected. | | |
| Moderate Clinical Significance - may be important to a healthcare | Moderate | 2 |
| professional as knowledge of a predisposition may aid diagnosis, | | |
| although prevention and treatment options may be limited. | | |
| May be able to limit morbidity with knowledge of predisposition. | | |
| May also be phenotype that is fatal with mortality that may not be | | |
| preventable or delayable but knowledge of | | |
| predisposition may aid diagnosis. | | |
| May be carrier of a monogenic phenotype with | | |
| a CSR = 3-4 when affected | | |
| High Clinical Significance - may be highly important to a healthcare | High | 3 |
| professional, may be a clinically serious phenotype whose diagnosis | | |
| may take significant time (months to years to decades) to make | | |
| without prior knowledge of predisposition. While prevention and/or | | |
| treatment options may exist, the phenotype may not be fully | | |
| preventable but may be able to delay onset or significantly limit | | |
| morbidity and/or mortality. | | |
| Critical Clinical Significance - may have critical importance to a | Critical | 4 |
| healthcare professional, may aid the prevention and/or diagnosis of a | | |
| very clinically serious phenotype, such as one that may cause sudden | | |
| death. Phenotype or phenotype sequela usually preventable, | | |
| manageable, or treatable. May be able to limit morbidity and/or | | |
| mortality if predispotion or affected status is known for the | | |
| phenotype. May be able to fully prevent or cure, either phenotype or | | |
| phenotype sequela, if predisposition or affected status is known. | | |

Each phenotype can have a separate CSR rating assigned to it (for example, by a licensed physician) for an increased risk of that phenotype and for a decreased risk of that phenotype. For monogenic phenotypes, each phenotype has a separate CSR rating assigned to it for the carrier state and for the affected or likely affected state (monogenic phenotypes with variable or low penetrance or expressivity may be designated as ‘likely-affected’ instead of affected, because the manifestation of the phenotype and the degree of phenotype severity may have variability). The designation of carrier or affected is based on whether or not the genetic variant(s) associated with that phenotype are recessive or dominant in terms of Mendelian inheritance. Co-dominance and incomplete dominance may both be associated with unique phenotypes in the heterozygous state and those phenotypes will have their own CSR. A sample of phenotypes and their associated CSR ratings can be seen in FIGS. 6E-G.

For example, for the hair color phenotype, the CSR rating is “0” because hair color does not have clinical significance. However, for Long QT Syndrome, which causes of sudden death due to cardiac arrhythmias, this phenotype is assigned a CAR rating of “4” if the person is most likely affected with the syndrome because this information may require immediate attention by a healthcare professional. Alternatively, if the person is a carrier of a genetic variant associated with Long QT Syndrome but is not affected by the syndrome, then this has less clinical significance and is assigned a rating of “2” because it is moderately useful (a healthcare professional may find this information useful in terms of educating their patient about the risk their children (or future generations) may have in regards to Long QT Syndrome and also in educating the patient that a family relative may carry or be affected by this syndrome and therefore they may want to discuss this with them and have the family talk with their physicians about this, as the family members may want to undergo genetic testing and/or analysis as well). Clinical significance and relevancy takes into account multiple factors (for example, by a licensed physician), such as whether or not a healthcare professional will find the information about a risk or predisposition or carrier status (including carrier, affected, or likely affected) for a specific phenotype useful. For example, the phenotype Amyotrophic Lateral Sclerosis (ALS) has very scarce preventive measures available and only limited treatment options. However, the phenotype may be difficult to diagnose at times, as it may take months or years before the proper diagnosis is made. Because of this, increased risk of ALS may be assigned a CSR=2, as it may be of moderate importance to a healthcare provider as it may speed diagnosis and therefore limit the psychological turmoil that exists in patients with an illness of unknown etiology. A speedier and more efficient diagnosis may also limit the stress and psychological turmoil to the patient's family as well as the financial impact to the patient and the overall medical system, such as due to decreased physician visits or decreased number of tests or medications or both that are not specifically targeted at the true causative phenotype (the accurate diagnosis). Decreased risk of ALS may be assigned a CSR=1, as ALS is already a rare phenotype so protection (decreased risk) against a rare phenotype has only limited clinical significance as it may help direct the healthcare professional away from ALS if their patient has a neurologic disease of unknown etiology and therefore knowledge of a decreased risk of ALS may be of marginal benefit to a healthcare professional. As another example, for the monogenic phenotype Arrhythmogenic Right Ventricular Cardiomyopathy (ARVC, also known as Arrhythmogenic Right Ventricular Dysplasia), a healthcare professional may most likely find knowledge of a patient being affected by this phenotype (carrier status=affected or likely affected) as being of critical clinical significance because this phenotype may cause sudden death, it may cause sudden death as its presenting symptom, and also because there are numerous preventive measures that can be implemented to limit or avoid the sequela from the phenotype (such as sudden death). If an individual is known to have an ARVC associated genetic variant (and is found to be affected or likely affected), this information may be tremendously empowering to a healthcare professional and may possibly lead to life-saving interventions and preventive measures. The CSR is similar to the GVP Triage but occurs at the phenotype level, while GVP Triage occurs at the genetic variant-phenotype level. This allows for the sorting and filtering of data at multiple levels, as well as threshold values to be implemented throughout the analytical process at multiple levels and augments operator control through providing multiple data filtering levels.

Other rating systems may include the Phenotype Impact Rating (PIR), such as shown in Table 7. The PIR is a rating scale that assigns an integer (such as an integer ranging from −3 to +3) to each phenotype based on the impact that phenotype may have upon the person. The PIR allows phenotypes beneficial to survival to be discerned efficiently from phenotypes that are detrimental to survival, and also phenotypes that are more beneficial or those that are more detrimental to be discerned efficiently from those that are less beneficial or detrimental. A separate PIR is assigned to monogenic carrier, monogenic affected, multifactorial decreased risk, and multifactorial increased risk. Polygenic phenotypes are assumed to follow a multifactorial model throughout the analytical process.

Each phenotype can have a separate PIR rating assigned to it (for example, by a licensed physician) for an increased risk of that phenotype and for a decreased risk of that phenotype. For monogenic phenotypes, each phenotype can have a separate PIR rating assigned to it for the carrier state and for the affected state. The designation of carrier or affected is typically based on whether or not the genetic variant(s) associated with that phenotype are recessive or dominant in terms of Mendelian inheritance. As an example, the phenotype of ‘Increased Longevity’ is assigned a “+3” if there is an increased risk of that phenotype. For a disease such as Crohn's Disease, if there is an increased risk of that disease then the PIR is “−2” because it is a very serious chronic disease but is usually not life-threatening. If there is a decreased risk of Crohn's disease, however, the assigned PIR is “+1” because it is slightly beneficial to be protected against this disease but since most people don't have Crohn's disease and since protection against Crohn's disease won't significantly augment or prolong life (or decrease the morbidity or mortality of any other diseases), decreased risk of this disease has less of an impact upon a person than an increased risk of the disease (which is why increased risk for Crohn's disease is assigned a “−2” while decreased risk is assigned a “+1”). The PIR is one of the components of the Action Score; because of this, one of the ways the Action Score is weighted is by how beneficial or how harmful that specific phenotype is.

TABLE 7
Phenotype Impact Rating (PIR)

| | Phenotype |
|---|---|
| Phenotypic Effect | Impact Rating (PIR) |
| Causes sudden death or severely debilitating disease | −3 |
| Serious disease or difficult to treat/cure condition | −2 |
| Manageable disease | −1 |
| Neutral phenotype | 0 |
| Slightly helpful trait or ability | +1 |
| Moderately helpful trait or ability | +2 |
| Significantly Advantageous trait or ability | +3 |

The aforementioned rating systems can be used in ranking genetic variants and phenotypes. For example, based on the ratings or rankings, genetic variants associated with phenotypes can be selected for analysis to generate a genetic profile and/or a genetic report tailored to a specific individual.

Analysis may include determining the Cumulative Genetic Risk (CGR) and the Predictive Medicine Lifetime Risk (PMR) for polygenic or multifactorial phenotypes by analyzing all (one or more) relevant (based on information known and rating systems applied, such as GVP Score and GVP Triage) genetic variants that are associated with that phenotype. The Cumulative Genetic Risk (CGR), also known as the Genetic Cumulative Risk (GCR), is the individual's cumulative genetic risk for polygenic or multifactorial phenotypes based on comprehensive analysis of their relevant genetic variants that are associated with the specific phenotype. Relevant genetic variant(s) can be selected based on those that make the cut-off threshold for analysis, as previously described. In many cases, genetic variants have three possible genotypes: Allele1/Allele1, Allele1/Allele2, or Allele2/Allele2. In some embodiments, the first step in calculating the CGR is to convert the odds ratios associated with the alleles or genotypes of all the relevant genetic variants associated with that specific phenotype into relative risks. In some embodiments, odds ratios are converted into relative risks as described by Zhang and Yu (JAMA 280:1690-1691 (1998)). The genotype frequency, from sources available in the arts, such as The International HapMap Project (http://www.hapmap.org)) for each of the three possible genotypes for each of the genetic variants is then multiplied by the relative risks for each of the three genotypes for each relevant genetic variant associated with that phenotype. The HapMap population used to ascertain these values is matched as closely as possible with the population of the individual who is currently undergoing genetic analysis (for example, if the individual is an European American, then the ‘CEU’ HapMap frequencies are utilized in the calculation). The resulting three values (genotype frequencies multiplied by relative risks for all three possible genotypes) for the genetic variant are then added together and produce a single number for each genetic variant. This value is then multiplied together for all relevant genetic variants detected during genetic testing and the resulting value is referred to as the Generic Population Risk Load (GPL). Next, the individual's genotype is considered at each of the relevant genetic variants and the relative risks associated with each of those relevant genetic variants (based on that genetic variant's genotype for that individual) are multiplied together to create a single value, known as the Proband Risk Load (PRL). The cumulative relative risk for an individual, also known as their Genetic Cumulative Risk (GCR) or their Cumulative Genetic Risk (CGR) is: CGR=PLR/GPL. An exemplary embodiment describing a method for determining a cumulative genetic risk for an individual is provided herein as Example 5.

The Predictive Medicine Lifetime Risk (PMR) is the new lifetime risk for an individual for polygenic or multifactorial phenotypes based on their gender-matched population specific Generic Lifetime Risk (GLR) and their own CGR. The PMR=(GLR)ₓ(CGR). Monogenic phenotypes are typically reported as a ‘carrier status’, which is analyzed and reported as non-carrier and non-affected, carrier but not affected, or affected. The degree to which the individual may be affected may also be reported, such as the potential age of onset, severity, penetrance or expressivity. An exemplary embodiment describing a method for determining a Predictive Medicine Lifetime Risk for an individual is provided herein as Example 5.

Utilizing this methodology, the genetic report may contain a comprehensive analysis of both risk, predisposition and carrier status for the individual. Some phenotypes, such as Alzheimer's Disease, are associated with both monogenic and multifactorial inheritance. In some cases, monogenic genetic variants may be analyzed as monogenic variants that may be deterministic of Alzheimer's Disease, while multifactorial variants that predispose to Alzheimer's Disease may be analyzed separately, as described herein for multifactorial phenotypes, and the results of the monogenic analysis and the multifactorial analysis may either be reported together or separately in the genetic report. The phenotype of Alzheimer's Disease may be represented as Alzheimer's Disease or the specific subtype of Alzheimer's Disease may be specified, such as Early-onset Alzheimer's Disease or Late-onset Alzheimer's Disease.

In some cases, a genetic report may contain information concerning an individual's risk of, predisposition for, or carrier status for two or more multifactorial phenotypes and two or more monogenic phenotypes. In some cases, a genetic report may contain information concerning an individual's risk of, predisposition for, or carrier status for: two or more multifactorial phenotypes; and one or more monogenic phenotypes, two or more monogenic phenotypes, three or more monogenic phenotypes, five or more monogenic phenotypes, ten or more monogenic phenotypes, twenty or more monogenic phenotypes, or fifty or more monogenic phenotypes. In some cases, a genetic report may contain information concerning an individual's risk of, predisposition for, or carrier status for: two or more monogenic phenotypes; and one or more multifactorial phenotypes, two or more multifactorial phenotypes, three or more multifactorial phenotypes, five or more multifactorial phenotypes, ten or more multifactorial phenotypes, twenty or more multifactorial phenotypes, or fifty or more multifactorial phenotypes. Sometimes, the number of multifactorial or monogenic phenotypes reported is “no more than” a certain number, e.g, no more than ten, no more than fifteen, no more than twenty; no more than thirty, no more than fifty, no more than one hundred, no more than two hundred, or no more than five hundred phenotypes.

Select genetic variants of clinical significance may be independently reported on or discussed in the genetic report. The genetic variants reported or discussed may be associated with monogenic or polygenic phenotypes or risk for multifactorial phenotypes. Some genetic variants may be included in the report, even if the predictive medicine risk or action score for that multifactorial phenotype is not included in the genetic report, such as if it does not make a certain threshold or cut-off value. For example, a single nucleotide polymorphism in the ITGB3 gene on (ITGB3 Chr. 17: 42715729 Y) is associated with premature coronary events and other phenotypes associated with premature heart disease and treatment effectiveness for heart disease. If the genotype for this SNP is found to convey increased risk of these phenotypes, the risk value for that genotype is applied to an algorithm, along with all other relevant genetic variants for that specific phenotype, but regardless of the AS or PMR for that phenotype, the genetic report may still specifically mention this genetic variant and its phenotype associations, as this SNP has been shown to be responsible for considerable morbidity and mortality and has clinical utility on its own. The determination of what multifactorial risk genetic variants are of special clinical utility and significance or the designation of genetic variants as having special clinical significance may be made by a licensed medical physician and can be automatically reported on (included) in the genetic report. Alternatively, genetic variants-phenotype associations with a specific GVP Triage level or phenotypes with a specific CSR may be chosen for inclusion within the genetic report regardless of the phenotypes ultimate AS or PMR.

Specific genetic variant(s) that are tested for whose allele(s) or genotype(s) deduced are found to not be associated with risk for a phenotype may also be included within the genetic report, so that the individual who ordered the genetic report, or their physician or other third party, is aware that the specific genetic variant or phenotype or both was tested for but the phenotype associated allele(s) or genotype(s) wasn't or weren't detected or no increased or decreased risk was ascertained based on the allele(s) or genotype(s) that were detected through the genetic testing and analysis. For example, if the individual is found to not have the major cystic fibrosis related deletion, referred to as the delta-F508 mutation (CFTR Chr. 7: 116986883-116986885 delTTT), then the genetic report may specifically indicate that this clinically significant genetic variant was not detected. A list of some or all genes or genetic variants or both tested for, regardless of whether or not their alleles or genotypes are associated with increased or decreased risk or no change in risk of a multifactorial phenotype or a carrier or affected of a polygenic or monogenic phenotype, as well as a list of some or all of the phenotypes tested for, may or may not be included in the genetic report and may or may not appear in a separate section of the genetic report. The genetic variants with the greatest significance, such as those that are more frequently the cause of, or are associated with, the phenotype, (such as those with higher overall phenotype-associated allele or phenotype-associated genotype frequencies or those associated with a higher population attributable risk) may be listed first or in a separate section compared to those genetic variants that appear less frequently (such as those with lower overall phenotype-associated allele or phenotype-associated genotype frequencies or those associated with a lower population attributable risk) as the cause of, or associated with, the phenotype in a single population, throughout multiple populations, or throughout all populations.

The Generic Lifetime Risk (GLR), as previously stated, is the gender-specific population lifetime risk for a specific phenotype prior to any genetic analysis, which may be represented as a percentage or be able to be converted to a percentage. This data can be obtained from published literature and from sources available in the arts including, but not limited to, published journal articles, national governmental health and disease services agencies or departments (such as the Health and Human Services in the United States or the National Health Service in the United Kingdom), including all of the agencies and divisions of the primary governmental health agency such as the United States Department of Health and Human Services (HHS) and all of its agencies and divisions including the United States' Centers of Disease Control and Prevention (CDC) and the United States' National Institutes of Health (NIH) as well as all its divisions, such as the National Cancer Institute (NCI). For example, the Generic Lifetime Risk at birth for Diabetes Mellitus, Type II for European Americans is 0.312 for females and 0.267 for males, for African Americans it is 0.490 for females and 0.402 for males, and for Hispanic Americans it is 0.525 for females and 0.454 for males (Narayan et al. JAMA 290(14):1884-1890 (2003)) As another example, the Generic Lifetime Risk for Melanoma at birth for European American's is 0.0173 for females and 0.0256 for males, for African American's is 0.0009 for females and 0.0007 for males, for Hispanic American's is 0.0058 for females and 0.0052 for males, for Asian American's is 0.0016 for females and 0.0017 for males, and for Native American's is 0.0024 for females and 0.0034 for males. (National Cancer Institute's Surveillance, Epidemiology and End Results (SEER), http://seer.cancer.gov/csr/1975_{—}2005/results_merged/topic_lifetime_risk.pdf).

The Generic Lifetime Risk can be dependent on the age of an individual. For example, the GLR for Lung Cancer for Hispanic Americans is 0.0363 for females and 0.0526 for males at birth and 0.0369 for females and 0.0548 for males at age 40, for African Americans the GLR for Lung Cancer is 0.0545 for females and 0.0775 for males at birth and 0.0569 for females and 0.0847 for males at age 40, for Asian Americans the GLR for Lung Cancer is 0.0428 for females and 0.0703 for males at birth and 0.0432 and 0.0719 for males at age 40, for Native Americans the GLR for Lung Cancer is 0.0487 for females and 0.0527 for males at birth and 0.0510 for females and 0.0575 for males at age 40, and the GLR for Lung Cancer for European Americans is 0.0652 for females and 0.0786 for males at birth and 0.0665 for females and 0.0819 for males at age 40. (National Cancer Institute's Surveillance, Epidemiology and End Results (SEER), http://seer.cancer.gov/csr/1975_{—}2005/results_merged/topic_lifetime_risk.pdf) Generic Lifetime Risk can be determined for gender-specific populations for each phenotype both from birth and at different ages. In some cases, phenotypes are described as a “susceptibility to”, or an “increased risk of”, this susceptibility or risk may refer to genetic variants that provide for an increased risk as compared to the ethnicity and/or gender and/or age matched population generic lifetime risk values. Protection against may refer to a decreased risk or no risk as compared to the ethnicity and/or gender and/or age matched population generic risk values.

Prevalence rates, incidence rates, and heritability for phenotypes can be obtained through sources available in the arts, such as, but not limited to published literature and various public resources, such as previously described, including the HHS and its CDC or the NCI. If exact gender-specific population statistics (incidence rates or prevalence rates or both for a phenotype) do not exist, then comparable statistics may be utilized, such as determined by a geneticist, an epidemiologist or a licensed physician. For example, incidence rates of phenotype A may not be known for African American males but it is known for African Americans in-general (females+males), this value would be used until a value specific for African American males is reported or obtained. In other embodiments, prevalence rates of phenotype B is not currently known for European American females but it is known for Western European Caucasian females, and this value is used until a value specific for European American females is reported or obtained.

The GLR and PMR can be used to calculate the Percent Change in Lifetime Risk. The Percent Change in Lifetime Risk calculates the percent change between the GLR for a phenotype (for example, ascertained from journal articles or published records, such as from the CDC or NCI, as previously described) and the calculated PMR. The formula for the percent change in lifetime risk is: Percent change in Lifetime Risk=((PMR-GLR)/GLR)×100.

The Notice Me Factor (NMF) allows for the conversion of a range of percent change in lifetime risk into a single integer congruent to the scale of integers utilized with the CSR and the PIR. This NMF is one component of the Action Score; because of this, one of the ways the Action Score is weighted is by the NMF which is, in turn, determined by the Percent Change in Lifetime Risk. This is used because while some phenotypes may have high clinical significance (and therefore have a high CSR) and also be very detrimental to a person's health (and therefore have a negative PIR), the genetic variants, when analyzed together, may not increase or decrease the lifetime risk of that disease significantly.

For example, for increased risk of diabetes mellitus, type II, the CSR=3 because diabetes mellitus, type II is a significant health issue whose negative effects can be either avoided or minimized through either preventive measures or early-detection and treatment and the PIR=−2 because it is a serious chronic disease. However, if the Predictive Medicine Lifetime Risk of diabetes mellitus, type II, is 49.1% for an Hispanic American male individual, this represents only an 8.14% increase over the Generic Lifetime risk of 45.4% for an Hispanic American male. This Percent Change in Lifetime Risk most likely is not of significance to a practicing healthcare provider and therefore it is assigned a low NMF (NMF=1). However, if the Predictive Medicine Lifetime Risk of diabetes mellitus, type II, was instead 64.3%, then this represents a 41.6% increase over the Generic Lifetime Risk and is much more likely to be significant to a practicing healthcare provider and therefore it is assigned a much higher NMF (NMF=10).

TABLE 8
Notice Me Factor (NMF)

| Percent Change | Notice Me Factor (NMF) |
|---|---|
| <−50 | 20 |
| −50 to −20 | 10 |
| −19.99 to −10 | 5 |
| −9.99 to −0.01 | 1 |
| 0 | 0 |
| 0.01 to 9.99 | 1 |
| 10 to 19.99 | 5 |
| 20 to 50 | 10 |
| >50 | 20 |

The Action Score (AS) is a combination of the Clinical Significance Rating (CSR), the Phenotype Impact Rating (PIR), and the Notice Me Factor (NMF). These three numbers allow for the action score to be weighted by clinical significance, phenotype benefit or harm, and also the degree to which a person's genetic profile affects their risk for that phenotype. The formula used to calculate the action score is: Action Score=CSR×PIR×NMF

The Action Score can allow both the healthcare provider and the individual to efficiently discern which phenotypes they need to focus on in terms of understanding, education, surveillance, treatment and/or preventive measures. The more negative the Action Score, the more significant the harmful risk is for a specific phenotype based on the person's genetic profile. The more positive the Action Score, the more significant the beneficial value is for a specific phenotype based on the person's genetic profile.

A color-coding system may be used in an individual's genetic profile. For example, a shade of a red color may be used to depict a significantly harmful phenotype, whereas a shade of a blue color may be used to depict a significantly beneficial phenotype. Table 9 illustrates some embodiments, however, other colors may be correlated with different AS ranges, and other AS ranges may be used.

TABLE 9A
Action Score Color Scheme

| Action Score (AS) | Action | Action Score Color |
|---|---|---|
| >60 | Very High | Navy Blue |
| 41 to 60 | High | Airforce Blue |
| 21 to 40 | Medium | Baby Blue |
| 11 to 20 | Low | Alice Blue |
| −10 to 10 | Nothing | Tea Green |
| −11 to −20 | Low | Seashell |
| −21 to −40 | Medium | Lavender Rose |
| −41 to −60 | High | Hollywood Cerise |
| <−60 | Very High | Crimson |

TABLE 9B
Action Score Color Scheme

| Action Score (AS) | Action | Action Score Color |
|---|---|---|
| >60 | Very High | Navy Blue |
| 41 to 60 | High | Airforce Blue |
| 21 to 40 | Medium | Baby Blue |
| 11 to 20 | Low | Alice Blue |
| 0 to 10 | Nothing | Cream |
| −10 to 0 | Nothing | Cream |
| −11 to −20 | Low | Seashell |
| −21 to −40 | Medium | Lavender Rose |
| −41 to −60 | High | Hollywood Cerise |
| <−60 | Very High | Crimson |

The risks for the specific diseases or traits or conditions (also referred herein as phenotypes), can also be used to determine scores for one or more specific organ systems, or medical specialties, such as, but not limited to those shown in FIG. 10 and listed in Table 10.

TABLE 10
Organ Systems/Medical Specialties
Organ Systems/Medical Specialties

Anesthesiology & Critical Care
Cardiology
Dental
Dermatology
Development & Learning
Ear, Nose & Throat
Endocrinology - Pancreas
Endocrinology - Thyroid
Endocrinology - Misc
Fertility
Gastroenterology & Hepatology
Geriatric's Health
Gynecology
Hematology
Immunology & Allergy
Infectious Disease
Laryngology
Men's Health
Metabolic & Rare Diseases
Musculoskeletal
Nephrology
Neurology
Newborn's Health
Nutrition, Exercise & Weight
Obstetrics & Fetology
Oncology - Reproductive Organs
Oncology - Lung
Oncology - GI
Oncology - Misc
Ophthalmology
Otology
Pediatrics & Neonatology
Pharmacology & Toxicology
Psychiatry
Pulmonology
Rheumatology
Sexuality
Surgery
Syndromes
Traits & Special Abilities
Urology
Vascular
Women's Health

For example, a cardiovascular score, which indicates the genetic health for an individual's cardiovascular system, can be determined by integrating the risk factors for each of the specific conditions and diseases affecting the cardiovascular system of an individual. For example, a Cardiovascular Panel Alpha can be used. Scores for organ systems or medical specialties can include the risk factors determined from the genetic profile and can further include information obtained from the individual such as through questionnaires, as described below. Organ systems or medical specialties can include cardiovascular; heart; lung; laryngology and dental; laryngology; dental; nutrition, exercise, and weight; otology; pediatrics and/or neonatology; pulmonology; anesthesiology and critical care; dermatology; development and learning; ear, nose, and throat; endocrinology; gastroenterology and hepatology; gastroenterology; hepatology; gall bladder; liver; thyroid; pancreas; gynecology; hematology; oncology; hematology and oncology; immunology; immunology and allergy; infectious diseases; metabolic diseases; metabolic diseases and rare diseases; rare diseases; men's health, musculoskeletal; neonatology; neurology; obstetrics; obstetrics and fetology; opthalmology; pharmacology, toxicology and anesthesiology; pharmacology; toxicology; anesthesiology; psychiatry; psychiatry and addictions; rheumatology; sexuality; sexuality; sexuality and fertility; sleep medicine; surgery; syndromes; traits and special abilities; urology and nephrology; urology; nephrology; vascular; geriatric health; gender-specific health and women's health, as well as any others that appear in Table 10.

A series of panels are described herein that aggregate genetic variants into comprehensive panels that provide information about an individual's risks and in some cases, options, in a targeted area. The panels of genetic variants provide a profile of the health and risks that detail not only one or more diseases or conditions but also the genetic variants associated with the efficacy of drugs that may be utilized to treat the diseases or conditions or the genetic variants linked to lifestyle choices that are linked to the disease. For example, there are genetic variants involving lifestyle, such as smoking, or eating particular foods, which increase or decrease one's risk of a disease based on another genetic variant. Thus, identifying these genetic variants and the related phenotype may allow one to alter his or her life and impact the ultimate result of one's genes. The panels are chosen to combine those genetic variants that will provide composite information about the genetic profile along with additional variants beneficial to the client's or doctor's assessment and/or use of the information. These panels are newly created and offer beneficial advantages that allow one to identify the optimal medical intervention, medication, dosage of a drug, or adverse impacts of a drug at an earlier stage and thus avoid serious delays in crucial treatment. The panels serve a variety of functions for analyzing a group of genetic variants of an individual and in some embodiments allow one to evaluate the suitability of an individual for therapeutics, suitability for medical interventions such as surgery, transplantations (donor or recipient), psychiatric treatment, or treatment associated with other medical specialties described herein; or identify the best candidates for career recruitment or training such as for military or police work. The panels, in some cases, aggregate diverse genetic variants to provide a valuable profile of individuals that allows significant benefits in their overall treatment or management of life choices to improve health and, in some instances, longevity.

The panels of genetic variants may be performed on an individual simultaneously or over periods of time depending on the outcome of some of the tests completed. For example, some panels may include variants considered to be reflex phenotypes that may be follow-on evaluations depending upon the outcome of a first phenotype. These reflex phenotypes provide useful additional screening of the genome to determine the presence of valuable variants that will contribute to earlier intervention and reduce wasted treatments or eliminate dead ends in therapy. Reflex testing and reflex phenotypes are further discussed herein.

The different organ systems or medical specialties can be represented by different panels, such as those in FIG. 15-18. The panels comprise groups of phenotypes, including conditions, traits, diseases, and disorders, and corresponding genes and loci that can be tested. In some cases, the panels may comprise arrays, probes, primers or sequences that may be used to determine an individual's carrier status or risk of, or predisposition for, a phenotype, such as a condition, disease, disorder or trait. For example, the panel may be a Full Genome Panel Alpha (FIG. 15), Full Genome Panel Beta, Pediatric Panel Alpha, Pediatric Panel Beta, Women's Health Panel Alpha, Women's Health Panel Beta, Men's Health Panel Alpha, Men's Health Panel Beta, Executive Panel Alpha, Executive Panel Beta, Golden Panel Alpha [Geriatric and Aging Panel Alpha], Golden Panel Beta [Geriatric and Aging Panel Beta], Carrier Screening Panel, Embryo and Fetus Panel Alpha, Embryo and Fetus Panel Beta, Female Fertility Panel, Male Fertility & Erectile Function Panel, Pregnancy Panel, Assisted Reproductive Technology Panel, Reproduction, Egg & Sperm Donor Screening Panel Alpha, Reproduction, Egg & Sperm Donor Screening Panel Beta, Sexuality, Mate Selection, Relationships and Marriage/Divorce Panel, Exercise, Fitness and Athletic Training Panel, Dietary, Nutrition & Weight Management Panel Alpha, Dietary, Nutrition & Weight Management Panel Beta, Longevity Panel Alpha, Longevity Panel Beta, Illness of Unknown Etiology Panel, Military and Armed Forces Panel Alpha (FIG. 16), Military and Armed Forces Panel Beta (FIG. 17), Law Enforcement/Forensic/Investigative Panel (FIG. 18), Emergency Panel, Cardiovascular Panel Alpha, Cardiovascular Panel Beta, Dermatology Panel, Gastroenterology Panel, Neurology Panel, Neurologic Disease of Unknown Etiology Panel, Mouth & Dental Panel, Surgery & Anesthesiology Panel, Transplant Panel, Gynecology Panel, Auditory Panel, Endocrinology Panel, Rheumatology Panel Alpha, Rheumatology Panel Beta, Urology & Nephrology Panel, Opthalmology Panel, Oncology Panel, Adult Psychiatry Panel, Pediatric Psychiatry Panel, Addiction Panel, Infectious Disease Panel, World Infectious Disease Panel, Pulmonology Panel, Sleep Medicine Panel, Palliative Care Panel, Insurance Panel Alpha, Insurance Panel Beta, HIV Panel, Autism Panel, Learning & Education Panel, Heart Failure Panel, Preterm Infant Panel, Newborn Panel Alpha, Newborn Panel Beta, Multiple Sclerosis Panel, Depression Panel, Schizophrenia Panel, Bipolar Panel, Eating Disorder Panel, Smoker's Panel, Drinker's Panel, Allergy and Atopy Panel, Pharmacology & Alternative Medication Panel, Miscarriage, Spontaneous Abortion, or Difficulty Conceiving Panel, Pain Panel, Breast Cancer Panel, Ovarian Cancer Panel, Lung Cancer Panel, Colorectal Cancer Panel, Prostate Cancer Panel, Skin Cancer Panel, Leukemia Panel, Lymphoma Panel, Gastric & Gastrointestinal Cancer Panel, Head & Neck Cancer Panel, Multiple Myeloma Panel, Sickle Cell Panel, Cystic Fibrosis Panel, Coronary Artery Disease Panel, Myocardial Infarction Panel, Lipid Level Panel, Blood Pressure Panel, Obesity Panel, Diabetes Mellitus (Type II) Panel, Diabetes Mellitus (Type I) Panel, Inflammatory Bowel Disease Panel, Gastrointestinal Disease of Unknown Etiology Panel, Viral Hepatitis Panel, Alzheimer's Disease Panel, Parkinson's Disease Panel, Seizure & Epilepsy Panel, Thyroid Panel, Osteoarthritis Panel, Rheumatoid Arthritis Panel, Systemic Lupus Erythematosus Panel, Gout Panel, Malaria Panel, Asthma Panel, Chronic Obstructive Pulmonary Disease Panel, Pulmonary Hypertension Panel, Polycystic Ovary Syndrome Panel, Stroke Panel, Autoimmune Panel, Behavior & Aptitude Assessment Panel, Kidney Transplant Panel, Liver Transplant Panel, Lung Transplant Panel, Stem Cell Transplant Panel, Infection Panel, Blood Flow, Thrombosis and Thromboembolism Panel, Sports Panel, Pathology & Tissue Repository Panel, Incarceration Panel, Research & Clinical Trial Panel, Close Living Quarters Panel, Rare Disease Screening Panel, Medical Procedure & Interventional Radiology Panel, Fibromyalgia Panel, Heartbeat/Arrhythmia Panel, Blood Panel, Dyslipidemia Panel, Death/Autopsy Panel. There are also Custom Panels, where an individual can choose any disease or trait from any of the panels described herein (such as FIGS. 15-18). An individual can choose different denominations, such as a Custom 10 Panel, which tests for 10 phenotypes or a Custom 20 Panel, which tests for 20 phenotypes. Custom panels can range from one phenotype to over 1,000 phenotypes.

The panel may also be a Custom Panel (see for example, FIG. 19), where an individual can choose any phenotype from any of the panels described herein (such as FIG. 15-18). An individual can choose different sets of any phenotypes from any of the panels or from a complete list of all phenotypes available, such as a Custom 10 Panel, which tests for 10 phenotypes or a Custom 20 Panel, which tests for 20 phenotypes. Custom panels can range from two phenotypes to over 1,000 phenotypes. Furthermore, an individual may choose any panel or set of panels for various other options (FIG. 20). For example, any panel or specific phenotype may be used for the Offspring Projection through the Combined Analyses of Different Individuals (OP-CADI) Option (which is further described herein). For the Only Decreased Risk Option, any panel or specific phenotype may be designated as “Protection Only” at the request of an individual or healthcare provider. This designation means that only phenotypes that show a lower risk value (protection against the phenotype) are utilized for the organ system color or are included in the Genetic Report or both. Those phenotypes that the individual is found to be at increased risk for may then not appear in the Genetic Report. For the Only Increased Risk Option; any panel or phenotype may be designated as “Increased Risk Only” at the request of an individual. This designation means that only phenotypes that show a higher risk value (higher risk for the phenotype) are utilized for the organ system color or are included in the Genetic Report or both. Those phenotypes that the individual is found to be at decreased risk for may then not appear in the Genetic Report. For the Specific Disease Exclusion Option, any phenotype(s) may be chosen to be excluded from being included in the analysis, and in the calculation of the organ system score and color, the genetic health score and color, and in the Genetic Report. For example, an individual may choose the Full Genome Scan Panel but indicate an Exclusion Option for Alzheimer's Disease and Amyotrophic Lateral Sclerosis. In this example, both Alzheimer's Disease and Amyotrophic Lateral Sclerosis risk is not reported in the Genetic Report. If the raw genotypic data is saved and identifiable, then the individual may choose to have this Exclusion Option revoked at a later time so that all phenotypes that were excluded are analyzed (which may incur an additional fee). If the individual's raw genotypic data is not identifiable, then new genetic material may have to be obtained and the genetic testing rerun at the laboratory (which may incur an additional fee).

The panels also describe various genes and loci that may be used to detect the risk of the various phenotypes, such as diseases or traits, but it should be clear that other genetic variations in other genes and loci that are correlated with the various phenotypes, such as diseases or traits, can also be used. In some embodiments, variants that are thought to be significant in determining a phenotype, may include, but not be limited to, those described in FIG. 22. Furthermore, the phenotypes, such as diseases or traits, listed may also be a general disease category, such as cancer, which may include a variety of types. For example, cancer may include Lung Cancer, Colorectal Cancer, Breast Cancer, Ovarian Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Brain Cancer, Leukemia, Lymphoma, Multiple Myeloma, as well as other cancers, a subset of the listed, or different variations of the cancers listed. An example is shown in FIG. 15.

The asterisk next to the “Metabolic Diseases and/or Syndromes” in FIG. 15-19, denotes the long list of metabolic diseases and syndromes that follows. The Metabolic Diseases and/or Rare Diseases and/or Syndromes may include at least one or more of the following: Frasier Syndrome, Mesangial Sclerosis, Cri-du-chat Syndrome, Cockayne Syndrome, Cerebrooculofacioskeletal Syndrome, De Sanctis-Cacchione Syndrome, Pyruvate Dehydrogenase (E1-alpha) Deficiency, Hermansky-Pudlak Syndrome, Wiskott-Aldrich Syndrome, Blau Syndrome, Usher Syndrome, Rett Syndrome, Atypical Rett Syndrome, PPM-X Syndrome, Angelman Syndrome, Macrocephaly/Autism Syndrome, PTEN Hamartoma Tumor Syndrome, Lhermitte-Duclos Syndrome, Bannayan-Zonana Syndrome, Cowden Disease, Bannayan-Riley-Ruvalcaba, WHIM Syndrome, Lesch-Nyhan Syndrome, Antley-Bixler Syndrome, Marfan Syndrome, Shprintzen-Goldberg Syndrome, MASS Syndrome, Weill-Marchesani Syndrome, Leigh Syndrome, Watson Syndrome, Neurofibromatosis, Neurofibromatosis-Noonan Syndrome, Barth Syndrome, Sudden Infant Death Syndrome, Sudden Unexplained Nocturnal Death Syndrome, Brugada Syndrome, Long QT Syndrome, Heart Block, Sick Sinus Syndrome, McCune-Albright Syndrome, TKCR Syndrome, Mitochondrial Complex I Deficiency, Alexander Disease, Cornelia de Lange Syndrome, Klippel-Trenaunay Syndrome, Bloom Syndrome, Angelman Syndrome, Noonan Syndrome, LEOPARD Syndrome, Rothmund-Thomson Syndrome, Rapadilino Syndrome, Baller-Gerold Syndrome, Aicardi-Goutieres Syndrome, Cree Encephalitis, Chilblain Lupus, Werner Syndrome, Loeys-Dietz Syndrome, Furlong Syndrome, Hurler Syndrome, Scheie Syndrome, Mucopolysaccharidosis, Pendred Syndrome, McKusick-Kaufman Syndrome, Bardet-Biedl Syndrome, Refsum Disease, Cold-Induced Autoinflammatory Syndrome, Muckle-Wells Syndrome, CINCA Syndrome, Teacher Collins Syndrome, Oculodentodigital Dysplasia, Syndactyly, Hypoplastic Left Heart Syndrome, Atrioventricular Septal Defect, Alagille Syndrome, Tetralogy of Fallot, Contractural Arachnodactyly, Congenital Wolfram Syndrome, Keratitis-Ichthyosis-Deafness Syndrome, Bart-Pumphrey Syndrome, Vohwinkel Syndrome, Waardenburg Syndrome, Craniofacial-deafness-hand Syndrome, Charcot-Marie-Tooth Disease, Dejerine-Sottas Disease, Roussy-Levy Syndrome, Conotruncal Anomaly Face Syndrome, DiGeorge Syndrome, Velocardiofacial Syndrome, Lymphedema-Distichiasis Syndrome, Yellow Nail Syndrome, Lymphedema, Papillon-Lefevre Syndrome, Haim-Munk Syndrome, Nail-Patella Syndrome, MASA Syndrome, CRASH Syndrome, Hydrocephalus, Partial Agenesis of Corpus Callosum, Hypotrichosis-Lymphedema-Telangiectasia Syndrome, Prader-Willi Syndrome, Jervell and Lange-Nielsen Syndrome, Wolf-Hirschhorn Syndrome, Miller-Dieker Lissencephaly, Noonan Syndrome, Costello Syndrome, Cardiofaciocutaneous Syndrome, Tuberous Sclerosis, Chediak-Higashi Syndrome, Nephronophthisis, Senior-Loken Syndrome, Alpha Thalassemia/Mental Retardation Syndrome, Juberg Marsidi Syndrome, Smith Fineman-Myers Syndrome, Chudley-Lowry Syndrome, Sutherland-Haan Syndrome, Walker-Warburg Syndrome, Muscular Dystrophy, Shah-Waardenburg Syndrome, Central Hypoventilation Syndrome, Hirschsprung Disease, Netherton Syndrome, van der Woude Syndrome, Popliteal Pterygium Syndrome, Cleft Lip and Palate, Greig Cephalopolysyndactyly Syndrome, Pallister-Hall Syndrome, Polydactyl), Acrocallosal Syndrome, Chanarin-Dorfman Syndrome, Shwachman-Diamond Syndrome, Aarskog-Scott Syndrome, Faciogenital Dysplasia, Trichorhinophalangeal Syndrome, Kartagener Syndrome, Mosaic Variegated Aneuploidy Syndrome, Premature Chromatid Separation Trait, Denys-Drash Syndrome, WAGR Syndrome, Zellweger Syndrome, Adrenoleukodystrophy, Smith-Lemli-Opitz Syndrome, PAPA Syndrome, Cerebral Dysgenesis Neuropathy Ichthyosis and Palmoplantarkeratoderma Syndrome, Kallmann Syndrome, Proud Syndrome, Partington Syndrome, Lissencephaly, Infantile Spasm Syndrome, Griscelli Syndrome, Conradi-Hunermann Syndrome, Chondrodysplasia Punctata, Waardenburg Syndrome, Waardenburg Syndrome/Ocular Albinism, Tietz Albinism-Deafness Syndrome, Pfeiffer Syndrome, Jackson-Weiss Syndrome, Antley-Bixler Syndrome, Trigonocephaly, Mental Retardation, Autism Spectrum Disorder, Osteoglophonic Dysplasia, Meckel Syndrome, Tetralogy of Fallot, Joubert Syndrome, Opitz G Syndrome, Coffin-Lowry Syndrome, Borjeson-Forssman-Lehmann Syndrome, Turcot Syndrome, Muir-Torre Syndrome, Cafe-au-lait Spots, Mitochondrial Neurogastrointestinal Encephalomyopathy Syndrome, 2-methyl-3-hydroxybutyryl-CoA Dehydrogenase Deficiency, Cabezas Syndrome, Spondylocarpotarsal Synostosis Syndrome, Larson Syndrome, Atelostogenesis, Boomerang Dysplasia, Mitochondrial Complex III Deficiency, GRACILE Syndrome, Fertile Eunuch Syndrome, Bartter Syndrome, Gitelman Syndrome, Bamforth-Lazarus Syndrome, Congenital Hypothyroidism, Rieger Syndrome, Iridogoniodysgenesis Syndrome, Ring Dermoid of Cornea, Omphalocele, Cutis Laxa, Robinow Syndrome, Brachydactyly, Otopalatodigital Syndrome, Melnick-Needles Syndrome, Frontometaphyseal Dysplasia, Periventricular Heterotopia, Kenny-Caffey Syndrome, Hypoparathyroidism-Retardation-Dysmorphism Syndrome, Cohen Syndrome, Craniofrontonasal Syndrome, Pfeiffer Syndrome, Crouzon Syndrome, Crouzonodermoskeletal Syndrome, Muenke Syndrome, Saethre-Chotzen Syndrome, LADD Syndrome, CATSHL Syndrome, Thanatophoric Dysplasia, Achondroplasia, Hypochondroplasia, Cystic Fibrosis, Ehlers-Danlos Syndrome, Rubinstein-Taybi Syndrome, Microphthalmia, Hoyeraal-Hreidarsson Syndrome, Fanconi-Bickel Syndrome, Immunodeficiency-Centromeric Instability-Facial Anomalies Syndrome, Usher Syndrome, DiGeorge Syndrome, Velocardiofacial Syndrome, Familial Febrile Convulsions, Walker-Warburg Syndrome, Muscle-eye-brain Like Disease, Pachyonychia Congenita, Steatocystoma Multiplex, Chondrodysplasia Punctata, Parkes Weber Syndrome, Capillary Malformation-Arteriovenous Malformation, Crouzon Syndrome, Jackson-Weiss Syndrome, Beare-Stevenson Cutis Gyrata Syndrome, Pfeiffer Syndrome, Apert Syndrome, Saethre-Chotzen Syndrome, Mitochondrial DNA-depletion Syndrome, Adrenoleukodystrophy, Peroxisome Biogenesis Disorder, Carney Complex Variant, Carney Complex, Trismus-pseudocamptodactyly Syndrome, Alpers Syndrome, Chromosome 22q13.3 Deletion Syndrome, Progressive External Opthalmoplegia With Mitochondrial DNA Deletions, Charcot-Marie-Tooth Disease, Dejerine-Sottas Syndrome, Roussy-Levy Syndrome, Werner Syndrome, Lethal Restrictive Dermatopathy, Hutchinson-Gilford Progeria Syndrome, Lipodystrophy, Dunnigan Partial Lipodystrophy, Alagille Syndrome, Joubert Syndrome, Senior-Loken Syndrome, Nephronophthisis, Fragile X Syndrome, Fragile X Mental Retardation Syndrome, Fragile X Tremor/Ataxia Syndrome, Troyer Syndrome, Birt-Hogg-Dube Syndrome, Nevo syndrome, Ehlers-Danlos Syndrome, Fuhrmann Syndrome, Ectodermal Dysplasia, Zlotogora-Ogur Syndrome, Homozygous 2p16 Deletion Syndrome, Fanconi Renotubular Syndrome, Down Syndrome, Turner Syndrome, McArdle's Disease, Hermansky-Pudlak Syndrome, ARC Syndrome, Simpson-Golabi-Behmel Syndrome, ABCD Syndrome, Waardenburg-Shah Syndrome, Cardiofaciocutaneous Syndrome, IPEX Syndrome, Donohue Syndrome, Rabson-Mendenhall Syndrome, LIG4 Syndrome, Andermann Syndrome, Saethre-Chotzen Syndrome, Cherubism, CHARGE Syndrome, Scott Syndrome, Alpha-1-Antitrypsin Deficiency, Tangier Disease, Liddle Syndrome, Cystic Fibrosis, Pseudohypoaldosteronism, Omenn Syndrome, Cartilage-Hair Hypoplasia, Metaphyseal Dysplasia, Anauxetic Dysplasia, Severe Combined Immunodeficiency, Papillon-Lefevre Syndrome, Haim-Munk Syndrome, Periodontitis, Osteolysis, Winchester Syndrome, FG Syndrome, Cerebrooculofacioskeletal Syndrome, Lethal Congenital Contractural Syndrome, Synostoses Syndrome, Tarsal-carpal Coalition Syndrome, Teunissen-Cremers Syndrome, Stapes Ankylosis Syndrome, Symphalangism, Lujan-Fryns Syndrome, Melas Syndrome, Cyclic Vomiting Syndrome, Mitochondrial Complex IV Deficiency, 3-alpha Methylglutaconic Aciduria, Diabetes-Deafness Syndrome, C (Opitz Trigonocephaly) Syndrome, Ectodermal Dysplasia, Majeed Syndrome, MELAS Syndrome, NARP Syndrome, Ataxia and Polyneuropathy, Striatal Necrosis, MERRF Syndrome, Seckel Syndrome, Primordial Dwarfism, Cortical Dysplasia-focal Epilepsy Syndrome, Timothy Syndrome, Knobloch Syndrome, Cleidocranial Dysplasia, Griscelli Syndrome, Joubert Syndrome, Senior-Loken Syndrome, Leber Congenital Amaurosis, Glucose Transport Defect of the Blood-brain Barrier, Meckel Syndrome, Niemann-Pick Disease, Crigler-Najjar Syndrome, Gilbert Syndrome, Familial Transient Neonatal Hyperbilirubinemia, Dubin-Johnson Syndrome, Carbamoylphosphate Synthetase I Deficiency, Gaucher Disease, Biotimidase Deficiency, Osteogenesis Imperfecta, Maple Syrup Urine Disease, Tay-Sachs Disease, Cystic Fibrosis, Mucolipidosis, Canavan Disease, GM2-Gangliosidosis, Sandhoff Disease, Norrie Disease, Al-Awadi/Raas-Rothschild/Schinzel Phocomelia Syndrome, Alexander Disease, Sialidosis, Galactosialidosis, Hurler Syndrome, Scheie Syndrome, Multiple Pterygium Syndrome, Hurler-Scheie Syndrome, IDUA Pseudodeficiency, Glycogen Storage Disease, Pompe Disease, Danon Disease, Hers Disease, Congenital Tufting Enteropathy, Nanopthalmos, Glycogenosis, May-Hegglin Anomaly, Xeroderma Pigmentosum, Myotonia Congenita, Fechtner Syndrome, Propionic Acidemia, Sebastian Syndrome, Malignant Hyperthermia, Epstein Syndrome, Tarui Disease, Atrioventricular Septal Defect, Hypoplastic Left Heart Syndrome, Polyglucosan Body Disease, McArdle Disease, Galactose Epimerase Deficiency, Hunter Syndrome, Phenylketonuria, Hyperphenylalaninemia, Fucosidosis, Galactosemia, Fabry Disease, 22q11.2 Deletion Syndrome, Glutamate Formiminotransferase Deficiency, Holocarboxylase Synthetase Deficiency, Multiple Carboxylase Deficiency, Peroxisome Biogenesis Disorder, Biotimidase Propionic Acidemia Deficiency, 3-Methylcrotonyl-CoA Carboxylase 2 Deficiency, Alkaptonuria, Ethylmalonic Encephalopathy, Chondrodysplasia Punctata, Campomelic Dysplasia, Ceroid Lipofuscinosis, Congenital Disorder of Glycosylation, Adrenoleukodystrophy, Primary Hypertrophic Osteoarthropathy, Carnitine Deficiency, Cardioencephalomyopathy, Pyruvate Carboxylase Deficiency, Holoprosencephaly, Polydactyl), Combined Oxidative Phosphorylation Deficiency, Glycerol Kinase Deficiency, Carnitine Palmitoyltransferase Deficiency, Porphyria, Carnitine-acylcarnitine Translocase Deficiency, Lissencephaly, Subcortical Laminal Heteropia, Deficiency of 3-Beta-hydroxysteroid Dehydrogenase, Adrenal Hyperplasia, Pseudohermaphroditism, Kostmann Disease, Menkes Disease, Occipital Horn Syndrome, Pitt-Hopkins Syndrome, Coproporphyria, Harderoporphyrinuria, Protein-losing Enteropathy-Hepatic Fibrosis Syndrome, Acute Intermittent Porphyria, Tyrosinemia, Paraganglioma Syndrome, Ornithine Transcarbamylase Deficiency, Hyperammonemia, Fucosyltransferase-6 Deficiency, Short-chain Acyl-coenzyme A Dehydrogenase Deficiency, Mevalonic Aciduria, Hyper-IgD Syndrome, Hyperimmunoglobulin D and Periodic Fever Syndrome, Fumarylacetoacetase Pseudodeficiency, 3-Methylcrotonyl-CoA Carboxylase 1 Deficiency, Leukoencephalopathy with Vanishing White Matter, Desmosterolosis, Malonyl-CoA Decarboxylase Deficiency, Argininemia, Hyperinsulinism-hyperammonemia Syndrome, Adenylosuccinase Deficiency, Argininosuccinic Aciduria, HMG-CoA Synthase-2 Deficiency, Isovaleric Acidemia, Glycine N-methyltransferase Deficiency, Glutathione Synthetase Deficiency, Farber Disease, Phosphoserine Phosphatase Deficiency, HMG-CoA Lyase Deficiency, 3-hydroxy-3-methylglutaric Aciduria, Very Long-chain Acyl-coenzyme A Dehydrogenase Deficiency, Trimethylaminuria, Pyruvate Dehydrogenase E1-beta Deficiency, Thymine-uraciluria, Cystathioninuria, Methylmalonic Aciduria, Porphyria Cutanea Tarda, Hepatoerythropoietic Porphyria, Hawkinsinuria, Dystonia, Gamma-glutamylcysteine Synthetase Deficiency, Sudden Infant Death with Dysgenesis of the Testes Syndrome, UV-sensitive Syndrome, Allan-Herndon-Dudley Syndrome, Posterior Microphthalmia with Retinitis Pigmentosa and Foveoschisis and Optic Disc Drusen, Pyruvate Dehydrogenase Phosphatase Deficiency, Donnai-Barrow Syndrome, Hartnup Disorder, Pyruvate Kinase Deficiency, Metachromatic Leukodystrophy, Combined SAP Deficiency, Tetrahydrobiopterin Deficiency, Fructosuria, Escobar Syndrome, Deficiency of Medium Chain Acyl-CoA Dehydrogenase, Acute Hepatic Porphyria, Delta-aminolevulinate Dehydratase Porphyria, Oligodontia-Colorectal Cancer Syndrome, Carnitine Palmitoyltransferase II Deficiency, Wolman Disease, Kennedy Disease, Xanthinuria, Cholesteryl Ester Storage Disease, Sea-blue Histiocyte Disease, Cerebrotendinous Xanthomatosis, Cartilage-Hair Hypoplasia, Anauxetic Dysplasia, Omenn Syndrome, Lecithin Cholesterol Acyltransferase Deficiency, Norum Disease, Fish-eye Disease, 3-methylglutaconic Aciduria, Erythrokeratodermia Variabilis, Deafness, Blindness, Gingival Fibromatosis, Hypodontia, Witkop Syndrome, Peroxisome Biogenesis Disorder, Batten Disease, GM1-gangliosidosis, Coenzyme Q10 Deficiency, Dolichol Kinase Deficiency, Melas Syndrome, Diabetes-Deafness Syndrome, Cyclic Vomiting Syndrome, Pontocerebellar Hypoplasia, Deficiency of Acid-labile Subunit, Dent Disease, X-linked Myopathy with Postural Muscle Atrophy and Generalized Hypertrophy, ACAD9 Deficiency, Pyridoxamine 5′-phosphate Oxidase Deficiency, C1q Deficiency, and Lowe Syndrome. Other metabolic diseases, syndromes or rare disorders may also be included.

In one aspect of the present invention, a set of panels are provided such as one or more of the panels in FIG. 15-18 that are directed to phenotypes, such as diseases, disorders, traits or conditions, that are gender specific. In some cases, gender-specific phenotypes, such as diseases, disorders, traits or conditions, are those that disproportionately affect one gender over another such, such as breast cancer or osteoporosis for females, and also for example X-linked diseases, such as, for example, Arts syndrome, Barth syndrome, and X-linked sideroblastic anemia. In other cases, gender-specific phenotypes, such as diseases, disorders, traits or conditions, are those that may only affect one specific gender such as for example endometriosis (female only), ovarian cancer (female only), prostate cancer (male only), or testicular cancer (male only). In still other cases, gender-specific diseases or conditions are those whose genetic predisposition or risk is affected by different genetic factors and/or phenotypes in males and females such as for example fertility, where female infertility may be associated with genes and genetic variants associated with thrombophilia and ovulatory defects while male infertility may be associated with genes and genetic variants associated with sperm morphology. Gender specific health, disease, or condition related genetic variants or phenotypes include but are not limited to Women's Health Panel Alpha, Women's Health Panel Beta, Female Fertility Panel Gynecology Panel, Polycystic Ovary Syndrome Panel, Men's Health Panel Alpha, Men's Health Panel Beta, Male Fertility & Erectile Function Panel, Urology & Nephrology Panel, Sexuality, Mate Selection, Relationships and Marriage/Divorce Panel. However, panels may be analyzed in a gender-specific manner, such as the Full Genome Panle Alpha (FIG. 15) that contains the ‘Cancer’ phenotype and will include ovarian cancer, endometrial cancer, and uterine cancer for only females and will include prostate cancer and testicular cancer for only males. Any phenotype that may affect both genders will be included for both genders, such as breast cancer, that even though it affects women at a greater frequency, it does still affects men, and therefore, for example, will be included under the ‘Cancer’ phenotype for both female and men in the Full Genome Panel Alpha (FIG. 15).

Each panel may be used to detect all the phenotypes (e.g., conditions, diseases, disorders, or traits) listed for each panel, such as the phenotypes listed for each panel, as shown in FIG. 15-18, or a panel may be used to detect a subset of phenotypes within the panel. For example, a panel may be used to detect at least 1, at least 2, at least 3, at least 4, at least 5, at least 6, at least 7, at least 8, at least 9, at least 10, at least 11, at least 12, at least 13, at least 14, or at least 15 phenotypes in a panel. In other cases, a panel is used to detect other numbers of phenotypes as provided herein. Thus, an individual or third party may choose to select one or more panels to determine the individual's risk or predisposition or carrier status for a specific phenotype or multiple phenotypes. The individual may have all the phenotypes in a panel analyzed for his or her genetic profile, or a select number.

Each panel may be used to detect only the phenotypes in bold as shown in FIG. 15-18, phenotypes in italics as shown in FIG. 15-18, or phenotypes in bold and italics as shown in FIG. 15-18. Each panel may also be used to detect subsets of the phenotypes in bold as shown in FIG. 15-18, subsets of the phenotypes in italics as shown in FIG. 15-18, or subsets of the phenotypes in bold and italics as shown in FIG. 15-18. In some cases, a panel may be used to detect at least 1, at least 2, at least 3, at least 4, or at least 5 of the phenotypes in bold, as shown in FIG. 15-18, or to detect at least 1, at least 2, at least 3, at least 4, or at least 5 of the phenotypes in italics, as shown in FIG. 15-18. In some cases, a panel may be used to detect at least 1, at least 2, at least 3, at least 4, at least 5, at least 6, or at least 7 or more of the phenotypes in bold and italics, as shown in FIG. 15-18. In some cases, a panel may be used to detect at least 1, but no more than 5, 6, 7, 8, 9, 10, 11, 12, 13, 14, 15, 16, 17, 18, 19, 20, 21, 22, 23, 24, or 25 of the phenotypes in a panel, as shown in FIG. 15-18. In some cases, a panel may be used to detect at least 2, but no more than 5, 6, 7, 8, 9, 10, 11, 12, 13, 14, 15, 16, 17, 18, 19, 20, 21, 22, 23, 24, or 25 of the phenotypes in a panel, as shown in FIG. 15-18. In some cases, a panel may be used to detect at least 3, but no more than 5, 6, 7, 8, 9, 10, 11, 12, 13, 14, 15, 16, 17, 18, 19, 20, 21, 22, 23, 24, or 25 of the phenotypes in a panel, as shown in FIG. 15-18. In some cases, a panel may be used to detect at least 4, but no more than 6, 7, 8, 9, 10, 11, 12, 13, 14, 15, 16, 17, 18, 19, 20, 21, 22, 23, 24, or 25 of the phenotypes in a panel, as shown in FIG. 15-18. In some cases, a panel may be used to detect at least 5, but no more than 10, 11, 12, 13, 14, 15, 16, 17, 18, 19, 20, 21, 22, 23, 24, or 25 of the phenotypes in a panel, as shown in FIG. 15-18.

Each panel may have probes for at least one genetic variant of the genes listed under the respective table, or may have at least one unique probe to detect each of the phenotypes in bold for a panel (as shown in FIG. 15-18), each of the phenotypes italicized for a panel, or each of the phenotypes bolded and italicized. The risk or predisposition or carrier status for one or more phenotypes in a panel may also be detected in an individual by other means, such as by sequencing. Each panel may have at least one exact position identifier within the entire genome (such as one or more of the following: a specific NCBI dbSNP rs number or exact chromosome and chromosomal position defined, for example, by Ensembl's coordinate numbering system or by exact sequence flanking or immediately flanking the genetic variant associated with the genetic variant of interest, such as about 5, 10, 15, 20, 25, 30, 40, 50 bp or more of sequence upstream or downstream of the genetic variant) for at least one genetic variant of the genes or loci listed under the respective table, or may have at least one exact position identifier within the genome (such as one or more of the following: a specific NCBI dbSNP rs number or exact chromosome and chromosomal position defined, for example, by Ensembl's coordinate numbering system or by sequence flanking or immediately flanking the genetic variant associated with the genetic variant of interest, such as 50 p of sequence upstream or downstream of the genetic variant) to detect each of the phenotypes in bold for a panel (as shown in FIG. 15-18), each of the phenotypes italicized for a panel, or each of the phenotypes bolded and italicized. There are also Custom Panels (see FIG. 19), where an individual can choose any phenotype (such as a condition, disease, disorder, or trait) from any of FIG. 15-18. For example, an individual may choose a Custom 10 Panel, which will test for 10 phenotypes the individual chooses, or a Custom 20 Panel, which will test for 20 phenotypes. The Custom Panel may have approximately, 5, 10, 15, 20, 25, 30, or more phenotypes. Custom panels can range from two phenotypes to over 1,000 phenotypes.

Each panel can test multiple genes and loci that are associated with traits and diseases that affect specific organ systems and areas of health care specialization. Organ systems and areas of health care specialization may include, but are not limited to, one or more of the following: cardiology and cardiovascular, laryngology and dental; nutrition, exercise, and weight; otology; pediatrics, neonatology; pulmonology; assisted reproductive technology specialization, anesthesiology and critical care; dermatology; development and learning; ear, nose, throat and dental; endocrinology; gastroenterology and hepatology; gynecology; hematology; oncology; immunology and allergy; infectious diseases; medical genetics, metabolic and rare diseases; men's health, military medicine, musculoskeletal; neurology; obstetrics and fetology; opthalmology; pharmacology, toxicology and anesthesiology; psychiatry and addiction; rheumatology; sexuality and fertility; sleep medicine; surgery; syndromes; traits and special abilities; urology and nephrology; vascular; and women's health, as well as any others that appear in Table 10.

Each panel can provide information on the risks or predispositions to one or more phenotypes, such as conditions, diseases or traits, for each organ system/healthcare or medical specialty individually to generate a Cumulative Action Score (CAS, further described below, also referred to as a System Score) and then together as a group, for example, to generate a total, overall, or cumulative genetic health score, as described further below. Each panel, or all of the panels, may be tested at a single time, for example, by using a single sample, such as a DNA sample or other genetic material. For example, thousands of polymorphisms and other genetic variants including, but not limited to, single nucleotide polymorphisms (SNPs), mutations, insertion/deletion polymorphisms (in/dels or DIPs), copy number variations (CNVs), repeats, translocations, inversions, and methylation status, within an entire genome can be detected. Both common and rare variants may be detected. Variants associated with monogenic phenotypes, polygenic phenotypes, or multifactorial phenotypes may be detected. Variants may be detected that indicate an individual carries a variant associated with a specific phenotype. Variants may be detected that indicate an individual is affected or is likely to be affected by a phenotype. Variants that increase risk and those that decrease risk can be detected and evaluated, also providing a more complete view of a person's overall genetic profile and genetic health. The genetic variants, such as polymorphisms, and phenotypes can be interconnected in a matrix. For example, a matrix may have just one dimension or may have two dimensions, the primary dimension being the phenotype matrix dimension (which shows how phenotypes are interconnected to each other) and then, superimposed upon this is the second dimension, the genotype matrix dimension (which shows how genetic variants and their alleles or genotypes are interconnected and how that dimension relates to the primary phenotype matrix dimension and any other matrix dimensions). The matrix may also have more than two dimensions. For example, a third dimension, superimposed upon the first two dimensions, may be the gene and loci matrix dimension (which shows how genes and loci are interconnected to each other and how that dimension relates to the primary phenotype matrix dimension and any other matrix dimensions), a fourth dimension may be the time matrix dimension and a fifth dimension may be the chronology matrix dimension. Each dimension, such as the phenotype matrix and the genotype matrix, contains multiple levels, with each level representing a degree of detachment from the primary phenotype or primary set of genetic variants and their alleles or genotypes. See, for example, FIGS. 13D, E.

The general disease names listed herein typically include all subsets of that disease. For instance, Alzheimer's Disease (AD) may refer to Late-onset AD, Early-Onset AD, Familial AD, or Sporadic AD. For Niemann-Pick Disease, that refers to all forms such as Type A, Type B, Type C, Type C1, Type C1 Adult Form, Type C1 Juvenile Form, Type C2, Type D (Nova Scotia Type), and so forth. This is applicable to all phenotypes listed herein.

Each phenotype, such as a condition, that is found to have either an increased risk or decreased risk may be factored into a genetic algorithm under one or more organ system/medical specialty categories. This links the results from the panel to a genetic analysis algorithm, which then computes the genetic health score for each organ system/medical specialty tested for within that panel and then an overall genetic health score (as discussed below). This information is then utilized to produce one or more genetic reports, which contains information including, but not limited to, preventive recommendations and/or interventions based upon the results of the comprehensive genetic testing results and analysis.

For example, if a decreased risk for osteoarthritis is found, then this decreased risk may be utilized within the genetic analysis algorithm and contribute to the genetic health score for ‘Rheumatology’ and the rheumatologic system. If an increased risk for myocardial infarction (heart attack) is found, then this increased risk is utilized within the genetic algorithm and contributes to the genetic health score for the ‘Cardiology’ or ‘Cardiovascular’ category, or organ system/medical specialty.

An organ system score, or medical specialty score, can be determined from at least 2 specific phenotypes, such as conditions, diseases or traits, of an organ system or medical specialty. Other organ system scores may be determined from at least 3, 4, 5, 6, 7, 8, 9, or 10 specific phenotypes, including conditions, diseases, disorders, or traits. An individual or third party, such as for example a medical professional, may choose to have carrier status or risks or both for a subset of phenotypes (also referred to herein as conditions, diseases or traits) listed in a panel to be determined. Alternatively, an individual or third party may choose to have one or more of carrier status or risks or predispositions for a subset of phenotypes, such as conditions, listed in a panel to not be determined or reported to them. For example, an individual may choose at least 2, 3, 4, 5, 6, 7, 8, 9, 10, 11, 12, 13, 14, 15, 20, 25, or all of the conditions of a panel to be analyzed or determined for their genetic profile. Alternatively, an individual may choose at least 2, 3, 4, 5, 6, 7, 8, 9, 10, 11, 12, 13, 14, 15, 20, or 25 of the conditions of a panel to not be analyzed or determined for their genetic profile.

An organ system score may be determined from a subset of the phenotypes, such as conditions, chosen or from all of the phenotypes, such as conditions. If a subset was chosen, the individual or third party may further choose to have carrier status or risks or both for other phenotypes, such as conditions, listed in a panel be determined after the initial risk or carrier status or both determination of a subset of phenotypes, such as conditions, listed on a panel, and the subsequent results can be added to the initial organ system score. Each phenotype, such as a trait, condition or disease, tested may be assigned to one or more of categories of organ systems or medical specialties (such as by a licensed physician) and such assignment can be factored into a genetic health score for each organ system/medical specialty. An overall genetic health score, described further below, can be determined using an algorithm that takes into account all of this information. An individual can be notified directly, or through a third party, on a recurring basis, such as for example every 3 to 6 months, or 6 months to yearly, or when the phenotype may become relevant (such as when the individual turns a specific age or when a specific milestone or event is met, such as for example if through genetic testing and analysis an individual is found to be at increased or decreased risk for West Nile Virus susceptibility and an increase in regional West Nile Virus infection cases occurs or an epidemic or pandemic occurs), about any updates, such as to changes in their predictive medicine score or their genetic health scores.

In some cases, the disclosure provides for monitoring of local, state, national, and/or international trends (e.g., rates of infection, increases in infection, decreases in infection, or outbreaks) of diseases, disorders or conditions such as, for example, HIV, HIV-1, HIV-2, West Nile Virus, Tuberculosis, Norwalk Virus, Meningococcal Disease, Pneumococcal Disease, Severe Acute Respiratory Syndrome, Legionnaires' Disease, Malaria, Leprosy, Typhoid, Dengue Fever, Aspergillosis, Toxoplasmosis, Epstein-Barr Virus, Salmonella, Schistosomiasis, Lyme Disease, or any infectious or transmittable disease or condition. Significant changes in local, state, national, and/or international trends may be associated with individuals who fit certain geographic criteria e.g., they reside or travel, or plan to reside or travel, in the local, state, or international area identified with the changing trend). Identified individuals who are found to be at increased or decreased risk for the infectious or transmittable disease, disorder or condition may then be notified of this change. The notification service may be offered for an additional fee, such as for example a subscription fee. The notification may include an updated genetic report, or updated predictive medicine score(s) or genetic health score(s).

In some embodiments, an individual may choose to have his or her predisposition, risk and/or carrier status determined for a subset of phenotypes, e.g., a subset of phenotypes listed in the Cardiovascular Panel Alpha, (e.g., coronary artery disease and myocardial infraction) or any other panel provided herein. A cardiovascular system score may be determined from this subset. The individual may further choose to have his or her predisposition, risk, and/or carrier status for other phenotypes, listed in the Cardiovascular Panel Alpha (or such as listed in both Cardiovascular Panel Alpha and Cardiovascular Panel Beta) to be determined after the initial risk and/or carrier status determination of the first subset of phenotypes (e.g., diseases, disorders, traits or conditions) was determined. The second set of results can be integrated into the initial cardiovascular system score to obtain a new score.

A “subset” may refer to any number of phenotypes (e.g., diseases, disorders, traits or conditions) less than the entire list of phenotypes, (e.g., diseases, disorders, traits or conditions) for a panel. In some cases, the subset of phenotypes (e.g., diseases, disorders, traits or conditions) can be tested separately from the subsequent set of phenotypes. An individual may submit a single sample to test for an initial subset of phenotypes, (e.g., diseases, disorders, traits or conditions) and submit a subsequent sample for subsequent phenotypes (e.g., diseases, disorders, traits or conditions). Alternatively, a single sample can be used to determine the carrier status, predisposition or risk of an individual for of all the phenotypes of a single panel, but only a subset of the results are reported to the individual initially.

A single sample may also be used to generate results from more than 1 panel. For example, a single sample may be used to generate results from 2 or more, 3 or more, 4 or more, 5 or more, or all of the panels.

Results from a subset of the panels may be reported. For example, all the phenotypes, such as conditions, of a subset of the panels (subset refers to any number of panels less than all the panels, including a single panel out of 2 or more panels) can be reported. Alternatively, a subset of the phenotypes (e.g., diseases, disorders, conditions or traits) of a subset of panels can be reported. Results from all the panels can also be reported to the individual. For example, all the phenotypes from all the panels, or a subset of phenotypes from all the panels can be used to generate a report. Phenotypes (e.g., diseases, disorders, traits or conditions) not reported initially can be subsequently reported, for example, after an individual consults with his or her physician, genetic counselor, physician assistant, nurse practitioner, other healthcare professional or other third party. Some examples of reporting a phenotype after an event subsequent to the initial genetic analysis, e.g., after the individual consults with a physician, are provided when the concept of “reflex testing” is described herein.

A single panel or combinations of the different panels may be used to generate a single organ system score. For example, phenotypes, such as conditions, in the Addiction Panel may be used in determining a pulmonary system score (such as nicotine addiction, lung cancer risk, and emphysema risk) and liver (hepatology) system score (such as liver disease due to alcoholism). Alternatively, a single panel can give rise to phenotypes, such as conditions, that can be applied to more than one organ system score. For example, if an increased risk or carrier status for Malignant Hyperthermia is found, then this increased risk or carrier status is utilized within the genetic analysis or algorithm or both and can contribute to the genetic health score for both ‘Anesthesiology & Critical Care’ and ‘Surgery’. If an increased risk for Attention Deficit Hyperactivity Disorder (ADHD) is found, then this increased risk is factored into the genetic analysis or algorithm or both and can contribute to the genetic health score for both ‘Psychiatry’ and also ‘Development & Learning’. If an increased risk for Melanoma is found, then this increased risk is utilized within the genetic analysis or algorithm or both and can contribute to the genetic health score for both ‘Dermatology’ and ‘Oncology’. Thus, different panels may also have overlapping phenotypes, such as conditions, for example, the Smoker's Panel may have phenotypes, such as conditions, diseases or traits, that overlap with the Addiction Panel.

The genetic profiles can have one or more organ system scores (for example, as shown in FIG. 10, or as listed in Table 10). For example, at least 2, 3, 4, 5, 6, 7, 8, 9, or 10 organ system scores may be determined from a genetic profile. The organ system score can be selected by an individual or their health care provider or other third party. Selection can be based on an individual's consultation with one or more of the following: his or her genetic counselor, a managing doctor, a nurse practitioner, a physician assistant, a healthcare provider, a parent or legal guardian such as if the individual is a minor, a health care proxy, an advisor, or another third party. The score can be indicated numerically or by color, as described above. The score, or color, can be a Cumulative Action Score (CAS) or an Indicator of Genetic Health of Organ System. For example, in one embodiment, the color red would be used for scores less than −10 for an individual's genetic profile, indicating highly important to discuss with individual and may be highly important for individual to follow-up with their physician or specialist based on this information. Pink can be used for scores between −1 to −10 to indicate moderately important risk. Green can be used for scores of 0 to indicate no pertinent deleterious or protective information discovered although organ system was accessed. Blue can be used for scores between +1 to +10, to indicate moderately important protection. Gold can be used for scores >+10 indicating very beneficial protection, and no color can be used for an Organ System or Medical Specialty that was not accessed, for example, if an individual chose a genetic testing panel or package that did not contain information about this system or specialty.

In one embodiment, the CAS is calculated by adding all the individual Action Scores for all the phenotypes that fall under the same Medical Specialty or Organ System (for example, the list of Medical Specialties and Organ Systems as depicted in FIG. 10 or Table 10). To calculate CAS, the following formula may be used for N number of Action Scores, with the minimum value that N can be is equal to 1, is: CAS=(AS₁+AS₂+ . . . AS_{N})/N. If there is only one action score (N=1), then the formula is CAS=AS₁/1=AS₁.

Each Action Score can be calculated such that each AS has a CSR, a PIR, and a NMF integrated into it, and as a result, the score is weighted in terms of clinical significance, degree of phenotype benefit or harm, and significance of the change in risk. Therefore, each individual Action Score may be added together and divided by the total number of Action Scores available that are applicable for that specific medical specialty or organ system. A single action score can be applicable to one or more medical specialties or organ systems.

The CAS is also known as the System Score because it gives a score to each organ system and medical specialties that apply to the body. The System Score can be used in determining the organ system of greatest and least concern in terms of significant harmful risk for an individual and in terms of significant decreased risk for an individual. A System Score may be calculated for each organ system (that can also be defined in terms of a medical specialty) and a System Color can be assigned to that organ system, such as depicted in Table 11. Other coloring schemes can also be used, as well as other system score ranges may also be used. The coloring system can efficiently convey the organ systems and medical specialties of greater concern and those that are of lesser concern. For example, a genetic profile may be found to have significantly increased risks for stroke, Alzheimer's Disease, and migraines and therefore the neurological system (under the medical specialty Neurology) has a more negative System Score and its relevancy can be conveyed through a shade of red coloring. The System Score and the System Color can also be altered or changed with a change in environmental factors, such as quitting smoking or losing weight and this change or potential change may be conveyed in the genetic report.

The coloring can appear throughout a report for an individual's genetic profile, such as on tabs for each organ system and medical specialty, on a face or cover of the genetic report or one of the initial pages that displays a picture of the entire human body, with each organ system shaded by its System color and its score may also be indicated, or the coloring may appear in other locations throughout the report. The System Color can represent an indicator of the health of each medical specialty or organ system based on the person's genetic profile. For organ systems and medical specialties that are not accessed in that panel, no coloring appears for the System Color.

TABLE 11
Color Scheme for System Score

| System | | |
|---|---|---|
| Score | System Color | Relevency |
| >60 | Navy Blue | Highly Beneficial or Protective |
| 41 to 60 | Airforce Blue | Beneficial or Protective |
| 21 to 40 | Baby Blue | Moderately Beneficial or Protective |
| 11 to 20 | Alice Blue | Slightly Beneficial or Protective |
| −10 to 10 | Tea Green | No pertinent risk or protective information |
| | | discovered although medical specialty or |
| | | organ system was accessed. |
| −11 to −20 | Seashell | Slightly Deleterious or Harmful |
| −21 to −40 | Lavender Rose | Moderately Deleterious or Harmful |
| −41 to −60 | Hollywood | Deleterious or Harmful |
| | Cerise | |
| <−60 | Crimson | Highly Deleterious or Harmful |
| No Score | No Color | Medical specialty or organ system was not |
| Available | | accessed |

The panels that can provide genetic phenotype, such as condition, predisposition risks or carrier status or both for each organ system/healthcare specialty individually and can be grouped together to generate a total, overall, or cumulative genetic health score, based on all genetic organ/specialty scores combined (described further below). As described herein, thousands of genetic variants and polymorphisms, including but not limited to, single nucleotide polymorphisms (SNPs), mutations, insertion/deletion polymorphisms (in/dels or DIPs), copy number variations (CNVs), repeats, translocations, inversions, gene expression levels and methylation status, can be detected at a single time. Variants that increase risk and those that decrease risk can be evaluated, as well as variants that are associated with either being a carrier of a phenotype or having or likely having a phenotype can be evaluated, providing a more complete view of a person's overall genetic health. The thousands of genetic variants, such as polymorphisms, and their associated phenotypes can be interconnected in a matrix, as previously discussed (see for example, FIGS. 13D, E) and the matrix can be assessed and analyzed for each individual based on reflex testing (see for example, FIG. 13A-C) (reflex testing is further described, herein).

The organ system scores, CAS, or results from the panels, can also be used to generate a genetic health score. The overall genetic health score can be generated from one or more phenotypes such as the phenotypes in a panel, a subset of the phenotypes in a panel, the phenotypes in a group of panels, a subset of phenotypes in a group of panels, or for a number of organ systems, medical specialties. All the Cumulative Action Scores that are calculated can be added together to obtain a Genetic Health Score, for all organ systems and medical specialties, which is an overall genetic health score, an indicator of genetic wellness. The indicator can be a word, such as high, medium, or low, or ranging from extremely good, good, neutral, poor, extremely poor. The genetic health score can be a number, for example, ranging from 0 to 5, wherein 0 indicates an extremely poor genetic wellness, which indicates a high risk to serious disease or condition and a 5 indicates an extremely high genetic wellness, indicating extremely low risk of medical conditions. The genetic health score can also be a percentage, such as a high percentage indicating a high likelihood or risk of disease and a low percentage indicating a low likelihood or risk of disease. Genetic health score or genetic wellness can also be expressed in a range of colors, for example, red indicating a high risk of having poor general health or predisposition to poor general health, yellow for average, and blue for an extremely high genetic wellness, with low risk of having diseases or conditions.

In some embodiments, the Genetic Health Score is a single score that takes into account all System Scores that already have had all action scores factored into them. This provides for a single score that can be used to compare an individual's Genetic Health Score to others, as well as to see how an individual's Genetic Health Score changes over time with environmental factors, such as if an obese person institutes weight loss measures such as lifestyle modifications, such as dieting and exercise, or by taking medications, such as sibutramine, or by having surgery, such as gastric bypass surgery or gastric banding, and is able to significantly decrease their body mass index. As with the CAS, each Genetic Health Score range can have a specific color associated with it (Table 12). Other colors and score ranges may also be used. The formula used to calculate the Genetic Health Score for a N number of Cumulative Action Scores, with the minimum value that N can be is equal to 1, is: Genetic Health Score=(CAS₁+CAS₂+ . . . CAS_{N})/N.

TABLE 12
Color Scheme for Genetic Health Score

| Genetic Health Score | Color |
|---|---|
| >60 | Navy Blue |
| 41 to 60 | Airforce Blue |
| 21 to 40 | Baby Blue |
| 11 to 20 | Alice Blue |
| −10 to 10 | Tea Green |
| −11 to −20 | Seashell |
| −21 to −40 | Lavender Rose |
| −41 to −60 | Hollywood Cerise |
| <−60 | Crimson |
| No Score Available | No Color |

In some embodiments, the genetic analysis of the present invention may provide an aggregate score of the PMRs associated with a group of related phenotypes. For example, a set of phenotypes may be identified as related to longevity. Such phenotypes may include but are not limited to one or more of the phenotypes, two or more of the phenotypes, or five or more of the phenotypes listed in Cardiovascular Panel Alpha, Cardiovascular Panel Beta, Heart Failure Panel, Coronary Artery Disease Panel, Myocardial Infarction Panel, Heartbeat/Arrhythmia Panel, Blood Panel, Dyslipidemia Panel, Lipid Level Panel, Blood Pressure Panel, Stroke Panel, Blood Flow, Thrombosis and Thromboembolism Panel, Longevity Panel Alpha, Longevity Panel Beta, Insurance Panel Alpha, Insurance Panel Beta; Exercise, Fitness and Athletic Training Panel, Sports Panel, Obesity Panel, Dietary, Nutrition & Weight Management Panel Alpha, Dietary, Nutrition & Weight Management Panel Beta, Executive Panel Alpha, and Executive Panel Beta. The aggregate score may be calculated in the same manner as a cumulative action score as described herein. In some cases, the aggregate score may be referred to as a longevity score.

In another example, a set of phenotypes may be identified as related to gender specific health. Such phenotypes may include but are not limited to one or more of the phenotypes, two or more of the phenotypes, or five or more of the phenotypes listed in Women's Health Panel Alpha, Women's Health Panel Beta, Female Fertility Panel Gynecology Panel, Polycystic Ovary Syndrome Panel, Men's Health Panel Alpha, Men's Health Panel Beta, Male Fertility & Erectile Function Panel, Urology & Nephrology Panel, Sexuality, or Mate Selection, and Relationships and Marriage/Divorce Panel. The aggregate score may be calculated in the same manner as a cumulative action score as described herein. In some cases, the aggregate score may be referred to as a gender specific health score.

In another example, a set of phenotypes may be identified as related to reproduction or pediatrics. Such phenotypes may include but are not limited to one or more of the phenotypes, two or more of the phenotypes, or five or more of the phenotypes listed in Preterm Infant Panel, Newborn Panel Alpha, Newborn Panel Beta, Pediatric Panel Alpha, Pediatric Panel Beta, Embryo and Fetus Panel Alpha, Embryo and Fetus Panel Beta, Assisted Reproductive Technology Panel, Reproduction, Egg & Sperm Donor Screening Panel Alpha, Reproduction, Egg & Sperm Donor Screening Panel Beta, Carrier Screening Panel, Rare Disease Screening Panel, Autism Panel, Learning & Education Panel, Behavior & Aptitude Assessment Panel, Pregnancy Panel, and Miscarriage, Spontaneous Abortion, or Difficulty Conceiving Panel. The aggregate score may be calculated in the same manner as a cumulative action score as described herein. In some cases, the aggregate score may be referred to as a pediatrics score, a reproduction score, or a reproduction/pediatrics score.

In another example, a set of phenotypes may be identified as related to the military, suitability for military service, or suitability for a specific position or assignment (and/or non-suitability for a specific position or assignment) in the military. Such phenotypes may include but are not limited to one or more of the phenotypes, two or more of the phenotypes, or five or more phenotypes listed in Military and Armed Forces Panel Alpha (FIG. 16), and Military and Armed Forces Panel Beta (FIG. 17). The aggregate score may be calculated in the same manner as a cumulative action score as described herein. In some cases, the aggregate score may be referred to as a military score, military recruitment score, or military suitability score.

In another example, a set of phenotypes may be identified as related to the medical care. Such phenotypes may include but are not limited to one or more of the phenotypes, two or more of the phenotypes, or five or more of the phenotypes listed in Emergency Panel, Surgery & Anesthesiology Panel, Transplant Panel, Kidney Transplant Panel, Liver Transplant Panel, Lung Transplant Panel, Stem Cell Transplant Panel, Interventional Radiology Panel; Pathology & Tissue Repository Panel, Research & Clinical Trial Panel, Pharmacology & Alternative Medication Panel, Pain Panel, and Death/Autopsy Panel. The aggregate score may be calculated in the same manner as a cumulative action score as described herein. In some cases, the aggregate score may be referred to as a medical care score.

In another example, a set of phenotypes may be identified as related to the brain and nervous system. Such phenotypes may include but are not limited to one or more of the phenotypes, two or more of the phenotypes, or five or more of the phenotypes listed in Depression Panel, Adult Psychiatry Panel, Pediatric Psychiatry Panel, Schizophrenia Panel, Bipolar Panel, Eating Disorder Panel, Alzheimer's Disease Panel, Parkinson Disease Panel, Seizure & Epilepsy Panel, Neurology Panel, Neurologic Disease of Unknown Etiology Panel, Multiple Sclerosis Panel; Addiction Panel; Smoker's Panel, and Drinker's Panel. The aggregate score may be calculated in the same manner as a cumulative action score as described herein. In some cases, the aggregate score may be referred to as a brain and nervous system score.

In another example, a set of phenotypes may be identified as related to endocrinology and/or rheumatology. Such phenotypes may include but are not limited to one or more of the phenotypes, two or more of the phenotypes, or five or more of the phenotypes listed in Endocrinology Panel, Diabetes Mellitus (Type II) Panel, Diabetes Mellitus (Type I) Panel, Thyroid Panel, Rheumatology Panel Alpha, Rheumatology Panel Beta, Rheumatoid Arthritis Panel, Systemic Lupus Erythematosus Panel, Gout Panel, Autoimmune Panel, Fibromyalgia Panel, and Osteoarthritis Panel. The aggregate score may be calculated in the same manner as a cumulative action score as described herein. In some cases, the aggregate score may be referred to as an endocrinology score, a rheumatology score, or an endocrinology/rheumatology score.

In another example, a set of phenotypes may be identified as related to cancer or aging. Such phenotypes may include but are not limited to one or more of the phenotypes, two or more of the phenotypes, or five or more of the phenotypes listed in Oncology Panel, Breast Cancer Panel, Ovarian Cancer Panel, Lung Cancer Panel, Prostate Cancer Panel, Colorectal Cancer Panel, Skin Cancer Panel, Leukemia Panel, Lymphoma Panel, Gastric & Gastrointestinal Cancer Panel, Head & Neck Cancer Panel, Multiple Myeloma Panel, Golden Panel Alpha Geriatric and Aging Panel Alpha, Golden Panel Beta Geriatric and Aging Panel Beta, and Palliative Care Panel. The aggregate score may be calculated in the same manner as a cumulative action score as described herein. In some cases, the aggregate score may be referred to as a cancer score, an aging score, or a cancer/aging score.

In another example, a set of phenotypes may be identified as related to infectious disease and pulmonology. Such phenotypes may include but are not limited to one or more of the phenotypes, or two or more of the phenotypes, listed in Illness of Unknown Etiology Panel, Sickle Cell Panel, Infectious Disease Panel, World Infectious Disease Panel, HIV Panel, Malaria Panel, Viral Hepatitis Panel, Infection Panel, Incarceration Panel, Close Living Quarters Panel, Asthma Panel, Chronic Obstructive Pulmonary Disease Panel, Pulmonary Hypertension Panel; Pulmonology Panel, Cystic Fibrosis Panel, Allergy and Atopy Panel, and Sleep Medicine Panel. The aggregate score may be calculated in the same manner as a cumulative action score as described herein. In some cases, the aggregate score may be referred to as an infectious disease score, a pulmonology score, or an infectious disease or pulmonology score.

In another example, a set of phenotypes may be identified as related to gastroenterology. Such phenotypes may include but are not limited to one or more of the phenotypes, two or more of the phenotypes, or five or more of the phenotypes listed in Inflammatory Bowel Disease Panel, Gastrointestinal Disease of Unknown Etiology Panel, Gastroenterology Panel, Dermatology Panel, Mouth & Dental Panel, Auditory Panel, and Opthalmology Panel. The aggregate score may be calculated in the same manner as a cumulative action score as described herein. In some cases, the aggregate score may be referred to as a gastroenterology score.

In another example, a set of phenotypes may be identified as related to law enforcement. Such phenotypes may include but are not limited to one or more of the phenotypes, two or more of the phenotypes, or five or more of the phenotypes listed in Law Enforcement/Forensic/Investigative Panel (FIG. 18). The aggregate score may be calculated in the same manner as a cumulative action score as described herein. In some cases, the aggregate score may be referred to as an law enforcement score.

In some embodiments, an individual may view how his or her Predictive Medicine Risk for each phenotype, his or her action scores, his or her cumulative actions scores, his or her longevity score, his or her gender specific health score, his or her pediatrics or reproduction score, his or her suitability-for-military service score, his or her medical care score, his or her brain and nervous system score, his or her endocrinology/rheumatology score, his or her cancer or aging score, his or her infectious disease and/or pulmonology score, his or her gastroenterology score, his or her law enforcement score, and his or her genetic health score, or gender-specific health score, changes based on certain variables, such as if he or she follows preventive recommendations or interventions or the advice of his or her physician or other third party. For example, if an individual is found to be at an increased risk of lung cancer due to smoking and the individual was a smoker, the genetic report may show and the individual may be able to see how his or her genetic profile and risk values will change if he or she quits smoking, if he or she has regular exams, such as an annual check-up, by a pulmonologist or other physician, such as an internist, or both (the decrease in risk may be separate values for each preventive recommendation or intervention and the decrease in risk may also be a different separate value when two or more preventive recommendations or interventions are combined, such as for example if a cigarette smoker quits smoking and also has regular exams by a pulmonologist or other physician). This change in risk values may be static, such as being printed in the genetic report, or dynamic, such as when the individual is meeting with a genetic counselor or nurse practitioner or physician assistant or other third party or if they are reviewing their results on the internet, such as on a webpage. Thus, individuals may be able to see how risk values would change (which may be represented by changes in the number values of the PMR, the AS, the CAS, or the genetic health score, changes in their colors, or verbally conveyed by a healthcare professional or one or more of the above) by checking off boxes associated with specific preventive measures or verbally agreeing to follow or choosing certain preventive measures. The individual may be able to visualize these changes on a display, such as a computer screen, holographic image, monitor, or television. This may apply to any change in a non-genetic (environmental) factor (such as lifestyle habits including eating habits and sexual habits, addictions, medications taken or not taken, compliance with medical advice, etc.)

In some embodiments, an individual may view how their Predictive Medicine Risk for each phenotype, their action scores, their cumulative actions scores, and their genetic health score changes based on certain variables, such as if they change their lifestyle habits or if they do not follow the advice of their physician or other third party. For example, if an individual is not a smoker and found to be at an increased risk of lung cancer due to smoking and the individual is currently not a smoker, the genetic report may show and the individual may be able to see how their genetic profile and risk values will change if they start smoking, if they have don't have regular exams, such as an annual check-up, by a physician, such as an internist, or both (the increase in risk may be separate values for each potential change in their lifestyle habits or preventive recommendation or intervention that they choose not to follow and the increase in risk may also be a different separate value when two or more preventive recommendations or interventions are combined, such as for example if a non-smoker starts smoking and also stops having regular exams by a physician). This change in risk values may be static, such as being printed in the genetic report, or dynamic, such as when the individual is meeting with a genetic counselor or nurse practitioner or physician assistant or other third party or if they are reviewing their results on the internet, such as on a webpage. Thus, individuals may be able to see how risk values would change (which may be represented by changes in the number values of the PMR, the AS, the CAS, or the genetic health score, changes in their colors, or verbally conveyed by a healthcare professional or one or more of the above) by checking off boxes associated with specific preventive measures or verbally agreeing to follow or choosing certain preventive measures. The individual may be able to visualize these changes on a display, such as a computer screen, holographic image, monitor, or television. This may apply to any change in a non-genetic (environmental) factor (such as lifestyle habits including eating habits and sexual habits, addictions, medications taken or not taken, compliance with medical advice, etc.)

Prior to obtaining a genetic profile an individual may be “pre-tested”, for example as shown in FIG. 1. An individual (102) can directly contact a central location (104), or a health care practitioner's office or other facility providing genetic testing and/or analysis, regarding genetic testing and/or analysis and obtain pre-testing consultation. Pre-testing may include a confidential meeting between the individual and a physician, genetic counselor, nurse practitioner, physician assistant, nurse, other healthcare provider or other third party. During the “pre-test”, an individual can consult with the healthcare provider, such as a genetic counselor, physician assistant, nurse practitioner, physician, or other third party who may suggest what type of genetic profile the individual may want based on the individual's concerns or information from a questionnaire the individual fills out.

For example, presymptomatic testing information from a presymptomatic genetic testing/analysis questionnaire can be analyzed prior to genetic testing and/or analysis for an individual. The individual may be the individual for which a comprehensive genetic profile is to be generated, or may be the healthcare provider for the individual, or a parent, legal guardian, health care proxy, caretaker, caregiver, sibling, physician, genetic counselor, nurse practitioner, physician assistant, or other third party. The questionnaire may be administered in person, for example by a genetic counselor, physician or other healthcare provider or other third party. The questionnaire may also be filled out by computer and transmitted over a network or internet, such as through a website, email, or ftp to be incorporated into a comprehensive genetic profile. The questionnaire may also be filled out by phone or by hand on paper and mailed or faxed. The questionnaire may include any question regarding the individual's medical history, including family history, of known, presumed, suspected, or feared phenotypes, including diseases, disorders, conditions, or traits. The questions relating to medical phenotypes, such as conditions, and medical history may include questions relating to confirmed diagnoses, presumptive diagnoses, or suspected diagnoses. Similarly, the questions relating to family history may include questions relating to confirmed diagnoses, presumptive diagnoses, or suspected diagnoses. The questionnaire may also include questions about the individual's past and present medication use, or daily habits and lifestyle such as tobacco, alcohol, or caffeine use. An individual's exercise regimen, diet, and living environment, for example, as well as exposure to sun, pollution, radiation, may also be on the questionnaire. Past or present symptoms experienced by the individual may also be on the questionnaire. In some cases, the questionnaire may include questions about prior medical examinations, prior or suspected medical findings, or prior test results. Information from the questionnaires can also be used in generating a genetic profile. For example, information such as an individual's living with a smoker could increase the individual's risk for lung cancer when the individual has a genetic variant that predisposes the individual for lung cancer due to tobacco or cigarette smoke exposure.

Thus, when calculating an individual's risk or predisposition for a phenotype, specific condition or set of conditions, the computer system and genetic analysis algorithm may take into account factors concerning the individual, including but not limited to: gender, ethnicity, age, weight, environmental factors and lifestyle habits (e.g., risk seeking behavior, smoking, drinking, diet, sun exposure, living environment, domicile location, etc.), medications, alternative therapies (e.g., herbs, yoga, acupuncture), present medical symptoms, family history for a disease, disorder or condition (including confirmed, presumed, or suspected diagnoses), personal medical history (including confirmed, presumed, or suspected diagnoses), results from a physical examination, results from a medical test, answers from a questionnaire, or other phenotypes, such as a condition, disease, disorder, or trait, of an individual or other factor described herein.

The questionnaire may be specific to an individual's concerns. For example, the questionnaire may be a “Carrier Questionnaire”, for individual(s) interested in having children and who are concerned whether they and their significant others are carriers for specific genetic variants that predispose for certain phenotypes, such as conditions, that may affect their future children. Questionnaires may also be specific for individuals with known phenotypes, such as conditions, (for example, patients diagnosed with cancer interested in their response to different cancer treatments), individuals of a young age (for example, specific to babies or children, to be filled out by their guardians), individuals whom are elderly, individuals who are thinking of or who have joined or are in the military, individuals who or thinking about or who have or do travel internationally, individuals who are about to go to college or university or boarding school, individuals who are contemplating donating eggs or sperm, individuals who may purchase eggs or sperm, and individuals who are pregnant and want to have their fetus tested. The questionnaires to be filled out may be chosen by a physician, genetic counselor (GC), or other healthcare provider or other third party. Questionnaires can be chosen based on an initial written or verbal consultation between the individual and the GC, or other healthcare professional, either in-person, over the telephone, or via the internet such as through video conference or via e-mail, or a website.

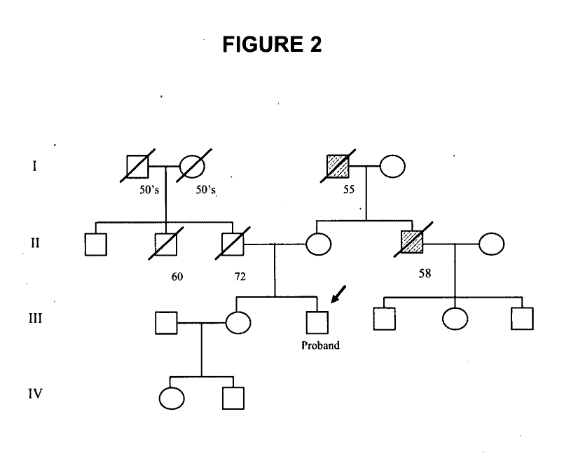

An individual's pedigree can be generated from the questionnaire (for example, as in FIG. 2). The genetic pedigree can be analyzed by a physician, genetic counselor, physician assistant, nurse practitioner, or other care provider and used in combination with other information from the questionnaire in determining what genetic testing, analysis or level of services should be performed. Based on the pre-testing (such as the results from the questionnaire or after consultation with a healthcare professional or both), an individual can decide what type of genetic profile or services he or she wants. The services can be customized to serve various cross sections of the society. For example, the phenotype, such as disease or condition, panels can be comprehensive, including many phenotypes, such as conditions, or limited, including one or two phenotypes, such as conditions. The number of phenotypes, such as diseases or conditions, offered can be determined by socio-economic need of an individual agreeing to receive comprehensive genetic analysis and a genetic profile. Other levels of service with varying costs to the individual can include genetic profiles with more than one phenotype, such as a disease or trait, amount of pre-test or post-test (follow-up) interaction with a health care provider or both, type of panel chosen, number of panels chosen, if OP-CADI is chosen, if reflex testing is chosen as well as the degree and depth of reflex testing chosen, or phenotypes chosen, such as diseases or traits, of an organ system or medical specialty, such as cardiovascular; dermatology; development and learning; ear, nose throat and dental; endocrinology; gastroenterology and hepatology; gynecology; hematology and oncology; immunology and allergy; infectious diseases; men's health, metabolic and rare diseases; musculoskeletal; neonatology; neurology; obstetrics; opthalmology; pharmacology, toxicology and anesthesiology; psychiatry and addiction; rheumatology; sexuality and fertility; sleep medicine; surgery; syndromes; traits and special abilities; urology and nephrology; vascular; and women's health.

Another level of service can be a comprehensive genetic profile or choice of panels, such as a Full Genome Analysis Alpha and Beta, or all panels. Other levels of service may depend on the type of panel chosen, such as those provided in FIG. 15-18. Other panels may be specific for testing the presence of various genetic variants for phenotypes, such as conditions, diseases or traits, of particular interest for a group of people. The individuals interested in the panels may choose to have their genetic profile determined from a single panel, a number of panels, or a subset of phenotypes, such as conditions and traits, from a panel. Alternatively, the individual may also choose phenotypes, such as diseases or traits, to make a Custom Panel (FIG. 19). For example, an individual may choose a Custom 10 Panel, which tests for 10 phenotypes, such as diseases, conditions or traits, the individual chooses, or a Custom 20 Panel, which tests for 20 phenotypes, such as diseases, conditions or traits. The Custom Panel may have approximately 3, 5, 10, 15, 20, 25, 30, or more phenotypes, such as diseases, conditions or traits. Thus, an individual can choose different denominations, such as a Custom 10 Panel, which tests for 10 phenotypes or a Custom 20 Panel, which tests for 20 phenotypes. Custom panels can range from one phenotype to over 1,000 phenotypes. An individual may make the choice after consultation with one or more of the following: GC, physician, nurse practitioner, physician assistant, or other healthcare provider or other third party. Reflex testing refers to the process wherein the determination of the risk, predisposition, or carrier status of an individual for one or more phenotypes, leads to, triggers, or causes another phenotype to be genotyped or not to be genotyped, to be analyzed or to not be analyzed, to be included in a report or not to be included in a report, to be included in a specific section of the report or not to be included in a specific section of the report, or any combination thereof.

The initial phenotype, such as a condition, disease, disorder, trait or addiction, may receive a positive or a negative result, and the reflex phenotype may be, but is not limited to, one or more of the following: a different phenotype, a phenotype related to the initial phenotype (e.g., indicator(s) of severity of initial phenotype, age of onset of initial phenotype, degree of penetrance or expressivity of initial phenotype (for example if the initial phenotype is coronary artery disease, the reflex testing may report on genetic variants that are indicators of the degree of severity of coronary atherosclerosis in coronary artery disease), a response to a type of treatment for the initial phenotype (e.g., adverse reaction to a medication used to treat or prevent the initial phenotype, ability to metabolize a medication used to treat the initial phenotype, indicators of what medications will or will not be most effective in treating or preventing the initial phenotype, dosing of medications to treat or prevent initial phenotype, outcome of surgery to treat the initial phenotype, or adverse reactions from surgery to treat the initial phenotype).

The predicted phenotype Outcome of Surgery includes whether or not the surgical procedure is likely to be successful in treating a disease or a symptom of a disease, either in the short-term or long-term of both. The initial phenotype may be a specific disease and the reflex phenotype may be a response to, or a sensitivity to, or effectiveness of, or adverse reaction to, a specific drug used to treat or prevent the disease. For example, an individual may be found to be at risk of breast cancer, and the reflex phenotype tested is the individual's response to, or sensitivity to, the drug tamoxifen. The results of the reflex testing of an individual's response to, or sensitivity to, tamoxifen may be reported simultaneously with the risk of breast cancer or may not be reported simultaneously but instead reported at a later time, such as if or when the individual is diagnosed with breast cancer.

In other cases, an individual may be found to be at risk of an initial phenotype that is an addiction and the reflex phenotype, such as a condition, to be tested is a disease or disorder that can result from the addiction. For example, an individual may be found to be at risk for nicotine addiction, which reflexes to the condition of risk of developing lung cancer due to smoking cigarettes or tobacco.

The reflex testing can be for risk of, predisposition to, or carrier status for more than one phenotype. For example, an individual may be found to be at risk for an initial phenotype such as having a heart attack (myocardial infarction) and, if so, an operator, or the information technology or computer system reflexes to testing multiple conditions related to the phenotype of myocardial infarction, such as, but not limited to: the risk of myocardial infarction with alcohol consumption, the likelihood that cardiovascular medications (e.g., anti-hyperlipidemic medications, anti-atherosclerotic medications, anti-restenosis medications) will be effective or will cause adverse reaction(s), the sensitivity of the individual to such medications, the carrier status or risk of decreased effectiveness (such as impaired antithrombotic action) of acetylsalicylic acid (aspirin), carrier status or risk of sensitivity, resistance, or adverse events with warfarin, starting dose indications with warfarin, the effectiveness of an oral antiplatelet agent, such as the platelet inhibitor clopidogrel or prasugrel or both, risk of stent thrombosis while on clopidogrel, risk of statin-induced rhabdomyolysis or myopathy, degree of cognitive decline after coronary artery bypass graft (CABG) surgery, or the likelihood of successful outcome following coronary angioplasty.

The results from such testing may help guide decisions as to, for example, what preventive measures the individuals should follow, what medication the individual should take, whether the individual should routinely take acetylsalicylic acid or other medication, whether the individual should follow a particular diet and/or exercise program, whether a particular surgery should be performed or alternative surgeries or treatments considered, what kind of medical screenings the individual should have, and the like.

Other examples of reflex testing are provided in FIGS. 15-18 and the disclosure herein, however, the present invention is not limited to those listed. The indications for reflex testing may not rely on genotypes of genetic variants but instead may be due to a quality or lifestyle or action or diagnosis or request of the person the genetic sample is from or the person, entity or third party ordering the test. For example, if the individual is a smoker then reflex testing may occur that will examine, analyze or report on lung cancer risk. Another example is if the individual spends a lot of time outside, such as for their profession, then reflex testing may occur that examines or reports on ultraviolet (UV) sensitivity and skin cancer risk. Yet in another example, an individual has a BMI above 25 or is overweight or obese, then reflex testing occurs that examines risk for diabetes. Another example is if the genotype of one or more genetic variants indicates that the individual is predisposed to uterine cancer but the individual has had a hysterectomy, then the predisposition for uterine cancer may or may not be reported or may be reported in a separate Risks to Relatives section of the genetic report but not in the section where the risk to the individual themselves is reported.

Another example is if the individual, medical professional, entity or third-party ordering the genetic profile indicates that they do not want to be tested for or notified of the results for a certain disease, such as Alzheimer's disease (AD), but genetic variants that increase the person's risk of Alzheimer's disease are found, then the reflex takes into account the request not to be notified (known as the ‘specific disease exclusion option’) and these results do not appear in the report or in the analysis of the neurologic organ system or the overall genetic health score or appear elsewhere and the results may or may not be stored in person's raw genotypic data or the person's raw analytic data that is either saved by the company conducting the genetic testing and analysis or by the person or entity or third-party or by the individual that the genetic sample was from or who ordered the test. The specific disease exclusion option may also be dependent upon the results of reflex testing. For example, an individual may indicate that they do not want to be notified of Alzheimer's disease only if the age of onset of AD is found to be likely before the age of 70 and the disease severity is found to be severe. Since age of onset of AD and severity of AD may be deduced through reflex testing, this individual's specific disease exclusion option is dependent upon the results of the reflex testing. This applies to all phenotypes and all options available, such as specific disease exclusion option, only decreased-risk option, only increased-risk option, and OP-CADI.

Reflex testing may also be a level of service that is provided. By testing for many different phenotypes, such as conditions, disorder, traits and diseases, including monogenic, polygenic, and multifactorial phenotypes, and by utilizing a robust and powerful database combined with genetic, heuristic, or other algorithms, reflex testing can be conducted in which a test result leads to operator-engagement or automatic engagement by an analysis system, such as a computer system, to examine other genetic variants of significance given those first results. Thus, if a significant result is found for a specific phenotype, such as a disease, disorder, trait or condition listed in FIGS. 15-18, Reflex Testing can automatically or manually report the associated phenotypes (e.g., diseases, disorders, traits or conditions) such as those shown in FIGS. 15-18. A schematic of reflex testing is depicted in FIGS. 13A-C. In some embodiments, there may be only a first and second round. In some cases, a positive or negative result for a first or an initial phenotype (e.g., disease, disorder, trait or condition) may reflex to the testing for a second phenotype (e.g., disease, disorder, trait or condition) and a positive, or negative, result for the second phenotype may reflex again to testing a third phenotype (e.g., disease, disorder, trait or condition) such as depicted in FIGS. 13A-C.

In some examples, the initial test result, phenotype, or genetic analysis may show either increased or decreased risk for a phenotype, such as a condition, or a carrier of a phenotype, or affected or likely affected by a phenotype. Other initial results may include having, being suspected of having, or being diagnosed with a phenotype, or having a family member that has or is suspected of having or is diagnosed with a phenotype. In other cases, or if an individual may be prescribed, or be taking, a certain medication or supplement. In other cases, an event that may trigger a reflex test may be that an individual reaches a certain milestone, such as a specific age or age range, or that an individual starts to go through puberty such as gonadarche, thelarche, or menarche, or when an individual starts attending school, or if an individual goes to a vocational school or boarding school or college or university or graduate school, or if an individual is thinking of joining or join a school sports team or non-school sports team or athletic club or is thinking of engaging in or are participating in an amateur or professional sport, or if the individual gets married, or is thinking about having children or trying to get pregnant, or when the individual starts or ends menopause or andropause, or if the individual dies, or if the individual is thinking of moving or moves to a different region such as a new state or country or continent or move from a rural to urban or from an urban to rural environment, or if there is a public health epidemiologic event, such as the changes in the incidence, prevalence or surveillance of a disease, such as Human Immunodeficiency Virus (HIV), Malaria, West Nile Virus (WNV), Cholera, Tuberculosis, diarrheal diseases, Small Pox, or Severe Acute Respiratory Syndrome (SARS), or such as an earthquake, flood, acts of terrorism or war, social or political unrest or other life event, community-level event, societal-level event or species-level event, or if the individual is suspected of committing a crime, or if the individual is arrested, or incarcerated, or if the individual becomes a consultant for or employee of the local or state or federal government, or if the individual joins the military.

In some cases, a positive result for a phenotype (e.g., disease, disorder, condition or trait) may reflex to testing for or analyzing a second or reflex phenotypes that is related to, or associated with, the first phenotype. In some examples, a positive result for risk for obesity may reflex to testing for diabetes mellitus type II risk, which, if found, may then reflex again to testing for medication metabolism and/or prognosis indicators associated with diabetes mellitus type II. In some cases, there may be a chain of three or more reflexes, so that an initial phenotype (e.g., disease, disorder, trait or condition) reflexes to a second phenotype (e.g., disease, disorder, trait or condition) or multiple second phenotypes (e.g., diseases, disorders, traits or conditions); a second phenotype, (or multiple second phenotypes) reflexes to a third phenotype, (or multiple third phenotypes); and a third phenotype reflexes to a fourth phenotype (or multiple fourth phenotype, such as depicted in FIG. 13) and so on. An initial phenotype (e.g., disease, disorder, trait or condition) may lead to testing, analyzing, and/or reporting a chain of 1 or more, 2 or more, 3 or more, 4 or more, 5 or more, 10 or more, 15 or more, or 20 or more reflex phenotypes (e.g., diseases, disorders, traits or conditions). For example, an initial phenotype may lead to testing, analyzing or reporting of 1, 2, 3, 4, 5, 6, 7, 8, 9, 10, 15, 20, 30, 50, 100, 200, or 500 reflex phenotypes (e.g., diseases, disorders, traits or conditions). In other cases, a negative result may be obtained for the initial phenotype (e.g., disease, disorder, trait or condition). The negative result may or may not be confirmed by repeating the test. The confirmation may or may not warrant any further reflex test.

Additional rounds of reflex testing may incur additional costs to an individual, or to his or her health care provider, or to a third party, such as an insurance provider. For instance, a low-cost service may be available whereas no reflex testing is available for any of the panel or phenotypes or both, a medium-cost service may be available where reflex testing goes only to round 2 and no further, and a high-cost service may be available where reflex testing goes through as many rounds as needed until no further reflex testing rounds exist. As another example, any additional reflex rounds beyond the initial first round may incur an additional fee, either all together or separately (each subsequent reflex round may represent another additional fee).

Reflex testing may be time independent or time dependent. For example, genetic analysis may identify an increased risk for coronary artery disease at time A and reflex testing for adverse reactions to HMG-CoA reductase inhibitors (Statins) can occur also at time A, automatically after the increased risk for coronary artery disease is detected. Alternatively, reflex testing for adverse reactions to HMG-CoA reductase inhibitors can occur at time B, which could be anywhere from instantaneous to years or decades after the initial increased risk for coronary artery disease is detected. For example, an individual being tested is a fetus or newborn, adverse reactions to HMG-CoA reductase inhibitors may not be important to that individual or the individual's family or healthcare providers at that time, but as that individual grows older and grows closer to the likely age of onset of coronary artery disease, such as 25 to 50 years later, then reflex testing may report on this individual's risk of adverse reactions to HMG-CoA reductase inhibitors. The increased risk of adverse reactions to HMG-CoA reductase inhibitors may take into account any new data, such as genetic variant-phenotype association data, and updated information that becomes available during the timeframe between the initial round and the subsequent reflex round of testing so that the reflex round risk analysis may change over time. The updated reflex analysis and reporting may also take into account any new data, such as new genetic variant-phenotype association data, and updated information in regards to the initial round of testing, such as for coronary artery disease in the example above, so that both the updated initial round of testing and the reflex round of testing (which may also be updated with the most recent information) will be reported at time B, after the initial genetic testing and analysis was conducted at earlier time A. The genetic testing analysis and reporting of results may be based on the initial DNA sample received from the individual, or a new DNA sample received at some later time, and may be based on the raw or already analyzed genetic testing data obtained from the initial genetic testing or from raw or already analyzed genetic testing data obtained from the individual since that initial time.

In an example of reflex testing), an individual may be found to be at increased risk of colorectal cancer (including, but not limited to colon cancer, rectal cancer, and/or colorectal cancer) through genetic testing or genetic analysis (such as of genetic information just obtained or obtained in the past) or diagnosed with colorectal cancer or a may have a possible diagnosis of colorectal cancer, or may have a polyp or other precancerous lesion association with colorectal cancer and this would then automatically or manually implement the reflex testing of sensitivity to or toxicity associated with chemotherapeutic medications used to treat colon cancer, such as irinotecan, and if a potential toxicity to a chemotherapeutic medication such as irinotecan is detected, then this may automatically or manually trigger reflex testing of the most appropriate starting dose of chemotherapeutic medications used to treat colon cancer, such as irinotecan (or an indication of the dose range, such as an indication to start at a lower dose due to possible toxicity at the usual starting dose or at a higher dose due to possible decreased effectiveness at the usual starting dose or at the usual starting dose due to no detected risk for toxicity, sensitivity, resistance, or decreased effectiveness at the usual starting dose).

In some embodiments, the initial reflex testing is for risk for colorectal cancer and the reflex testing is conducted after a diagnosis for colorectal cancer or after a precancerous lesion is detected. Such reflex testing may be both automatic or manual reflex testing and may determine potential toxicity or sensitivity to chemotherapeutic medications used to treat colon cancer, such as irinotecan, as well as to determine the most appropriate starting dose (or dosing indication range, such as start at a lower dose or at the usual dose or at a higher dose than is usually given) for chemotherapeutic medications used to treat colon cancer, such as irinotecan. In such examples, reflex phenotypes may be analyzed concurrently instead of one after the other.

In some cases, the initial reflex testing conducted after an increased risk for colorectal cancer is determined after a diagnosis for colorectal cancer is made or a precancerous lesion is detected, may be both automatic or manual reflex testing to determine potential toxicity or sensitivity to chemotherapeutic medications used to treat colon cancer, such as irinotecan as well as reflex testing to determine the most appropriate starting dose (or dosing indication range, such as start at a lower dose or at the usual dose or at a higher dose than is usually given) for chemotherapeutic medications used to treat colon cancer, such as irinotecan, so that these reflexes are analyzed concurrently instead of one after the other.

The individual's parent(s), legal guardian(s) or health care proxy or the individual, a healthcare provider or other third party (such as a school nurse, athletic coach, fitness trainer, insurance agent, a police officer or crime scene investigator) may be able to request a partial or full reflex analysis at any time or if certain events occurs or milestones are reached, so that, for example, at an early age such as for example 0, 1, 2, 3, 4, 5, 6, 7, 8, 9, 10, 12, 14, 15, 17, 18, 20, 21, 30 years or older, full reflex analysis can be analyzed and reported to the individual for phenotypes that may not affect that individual until they are older, such as coronary artery disease, Alzheimer's Disease, or prostate cancer. The genetic testing, analysis and reporting of results may be based on the initial DNA sample received from the individual, or a new DNA sample received at some later time, and may be based on the raw or already analyzed genetic testing data obtained from the initial genetic testing or from raw or already analyzed genetic testing data obtained from the individual since that initial time.

Reflex testing may automatically report relevant information to the individual, the individual's parents or legal guardians, the individual's health care proxy, the individual's physician or other healthcare provider, or a third-party, based on the age of the individual or other factors, such as if that individual is ever suspected of having or is diagnosed with a phenotype, such as a disease. This reporting may occur by a written update to the genetic report, an email, a text message, an auditory alert, a manual or an automatic addition to the individual's medical record, a facsimile transmission, a verbal communication over the telephone, internet, or in person, or through an internet conference or website. The individual or any third party receiving this reporting may be able to turn on or off automatic reporting as per their own preference. The genetic testing analysis and reporting of results may be based on the initial DNA sample received from the individual, or a new DNA sample received at some later time, and may be based on the raw or already analyzed genetic testing data obtained from the initial genetic testing or from raw or already analyzed genetic testing data obtained from the individual since that initial time. This manual or automatic reporting of reflex testing analysis may incur an additional fee.

In some examples, a patient or individual may choose to have only one level of reflex testing and/or analysis with his or her genetic analysis but, after reading the genetic report and, optionally, consulting with his or her healthcare provider, the patient or individual may decide to have further reflex testing and/or analysis or full reflex testing and/or analysis conducted that may then detect the carrier status, predisposition, or risk of said individual for one or more previously unreported phenotypes.

Even if a phenotype is not initially reported at time A, genetic testing and analysis, or genotyping on its own without any analysis (which gives raw genotypic data for one or more genetic variants or genes or chromosomes or the full exome or the full genome), may be conducted at time A, and the genetic analysis or genetic reporting or both containing information about both the initial round of analysis and carrier status and risk for those initial phenotypes as well as any information pertaining to reflex rounds, may not be reported to the individual, the individual's parents or legal guardians, the individual's health care proxy, the individual's physician or other healthcare provider, or a third party, until a later date or a specific milestone, which can be, such as for example, the individual's age or age range, or when an individual starts to go through puberty such as gonadarche, thelarche, or menarche, or when an individual starts attending school, or if an individual goes to a vocational school or boarding school or college or university or graduate school, or if an individual is thinking of joining or join a school sports team or non-school sports team or athletic club or is thinking of engaging in or are participating in an amateur or professional sport, or if the individual gets married, or is thinking about having children or trying to get pregnant, or when the individual starts or ends menopause or andropause, or if the individual dies, or if the individual is thinking of moving or moves to a different region such as a new state or country or continent or move from a rural to urban or from an urban to rural environment, or if there is a public health epidemiologic event, such as the changes in the incidence, prevalence or surveillance of a disease, such as Human Immunodeficiency Virus (HIV), Malaria, West Nile Virus (WNV), Cholera, Tuberculosis, diarrheal diseases, Small Pox, or Severe Acute Respiratory Syndrome (SARS), or such as an earthquake, flood, acts of terrorism or war, social or political unrest or other life event, community-level event, societal-level event or species-level event, or if the individual is suspected of committing a crime, or if the individual is arrested, or incarcerated, or if the individual becomes a consultant for or employee of the local or state or federal government, or if the individual joins the military or if they are suspected of having or are diagnosed with a phenotype, such as a disease.

For example, an individual's risk for attention deficit hyperactivity disorder may initially be detected through the genetic testing and/or analysis of a newborn but this information may not be reported. This information may then be reported at a later time, such as when the individual starts attending school, or if this individual experiences (or is suspected to have) learning difficulties or behavioral problems at school or elsewhere. Both the initial risk of attention deficit hyperactivity disorder and the reflex rounds of testing associated with this phenotype may then be reported to the individual, the individual's parents or legal guardians, the individual's health care proxy, the individual's physician or other healthcare provider, or a third-party.

The genetic testing analysis and reporting of results may be based on the initial DNA sample received from the individual, or a new DNA sample received at some later time, and may be based on the raw or already analyzed genetic testing data obtained from the initial genetic testing or from raw or already analyzed genetic testing data obtained from the individual since that initial time. Reflex testing may also be contingent upon actual diagnosis at earlier time A as the initial factor, such as if an individual is diagnosed by a healthcare provider, such as an internist or neurologist, as having epilepsy and then either genetic testing (the actual genotyping) or genetic analysis (of genotypic data) is conducted, or both, at later time B (which constitutes reflex testing because it is based off of the initial factor of a diagnosis of epilepsy) to ascertain risk or predisposition to resistance to antiepileptic drugs (AEDs) or dosing or sensitivity to AEDs, such as carbamazepine or phenyloin. As another example, an internist or rheumatologist may diagnose lumbar disc disease in a patient and then may want to ascertain the patient's pharmacogenomic profile for opiates, such as if the patient is resistant to the analgesic effects of opiates (effectiveness of opiates) or requires a lower or higher dose of opiates to obtain an analgesic effect, and if genetic testing (e.g., the actual genotyping) or genetic analysis (of genotypic data) or both is conducted for this phenotype in regards to (due to) the initial diagnosis, this constitutes reflex testing. The analysis of genetic information and the reporting of phenotypes or panels or both or the reporting of genetic variants or genotypes or both or the analysis of genetic variants and their associated phenotype(s) is not dependent upon time.

In some embodiments, a newborn may have his or her full genome sequenced and may have the raw data analyzed near or at that time when he or she is born, time A, or analyzed at a later time, time B, and reported at time B or reported at a later time, time C. For example, the newborn patient may have his or her full genome sequenced when he or she is born but his or her pediatrician may not order the Pediatric Panel Alpha until a later time, such as when the patient is five years old, Similarly, a newborn patient may have his or her full genome sequenced near birth, but the patient's cardiologist may not order the Cardiovascular Panel Beta for the patient until a later time, such as when the patient is about 1, 2, 3, 4, 5, 6, 7, 8, 10, 12, 14, 15, or 18, or years old, or when the patient is even older, such as when the patient reaches adulthood, is 18 or more years old, is over twenty, over twenty five, over thirty, over forty, over fifty, over sixty or over seventy years old. For example, an abnormality in a child patient's EKG is detected or a cardiac abnormality is detected during a clinical physical examination, which may then prompt the patient's health care provider (e.g., cardiologist) to order a Cardiovascular Panel.

When these panels are ordered at a later time, either the phenotypes and analysis may already have been conducted at an earlier time and therefore the results are just reported on and displayed at this later time, or the raw data is both analyzed and then reported on at this later time B or C. The analysis and reporting of panels and phenotypic information, both risk and carrier information, is therefore not time dependent upon when the actual genetic testing (e.g., the actual genotyping to ascertain the raw genotypic data) is conducted. The panel(s) to be analyzed and reported on can be chosen or paid for or both at the initial time of genetic testing (e.g., the actual genotyping when the raw genotypic data is obtained) or at a later time or both. If the raw genotypic data is ascertained at an earlier time and a panel is chosen and analyzed and reported on at a later time, either the original data concerning risk values of specific genetic variant-phenotype associations and carrier status may be used or updated data concerning risk values of specific genetic variant-phenotype associations and carrier status may be used. The original algorithm that was being utilized when the raw genotypic data was ascertained (e.g., from when the genetic testing was conducted) may be used or a new algorithm may be used. The genetic sample may also be obtained at a different time or at the same time as when the genetic testing (to ascertain the raw genotypic data) is conducted. This manual or automatic reporting of initial analysis of results or reflex testing analysis either at the time of the actual genetic testing or at a later time or both may incur an additional fee.

Genetic testing that ascertains an individual's (such as a person or an animal) genotype at one or more places in the genetic code may be conducted at time A and the genetic analysis or the genetic reporting or both may be conducted at a later time, time B. For example, full genome sequencing may ascertain an embryo's or newborn's genetic code and this genetic code may be analyzed or reported on or both, in part or in full, immediately or not until a later time, such as seconds, minutes, hours, days, weeks, months, years, or even decades in the future after the initial testing and/or analysis occurred. If specific groups of phenotypes or genes are tested for and/or analyzed and/or reported on, then that specific group of phenotypes or genes may constitute a specific panel, regardless of where or when the testing (genotyping, sequencing, or any other genetic testing methodology described herein), analysis, or reporting occurs.

The milestones that trigger the reporting of either the results of the initial round of genetic analysis and reporting or one or more reflex rounds of analysis and reporting or both at some time (either instantaneous or seconds to minutes to days to weeks to years to decades) after the initial genetic sample has been obtained (and either stored or genetic variants tested for or sequencing or full sequencing conducted so that raw genotypic data is obtained, such as genotypes at one or more positions within the genome) and preliminary analysis conducted (and either a report generated or no report generated or an abbreviated report generated with only some information) may be determined either by the service provider of the genetic analysis and genetic reporting or may be determined by the individual, the individual's parents or legal guardians, health care proxy, physician, genetic counselor, physician assistant, nurse practitioner, healthcare provider, or third party. Examples of milestones include: referral to, consultation with or ordering of a test or panel by a physician, specialist, genetic counselor, physician assistant, nurse practitioner, healthcare provider, insurance agent or third party, age or age-range, suspected diagnosis of a phenotype, such as a disease, diagnosis of a phenotype, such as a disease, having a family member that is suspected of having a phenotype, such as a disease, having a family member that is diagnosed with a phenotype, such as a disease. A life-event, such as puberty, gonadarche, thelarche, menarche, or death, prescribing of medication, start attending school, applying to or attending a vocational school or boarding school or college or university or graduate school, planned or actual participation in a school sports team or non-school sports team or athletic club, planned or actual participation in an amateur or professional sport, attempting to get pregnant, getting pregnant, having a child, suspected of committing crime, being arrested, being incarcerated, becoming a consultant for or employee of the local or state or federal government, first sexual experience, marriage, divorce, planning to join the armed forces, enlistment in armed forces, employment, travel, temporary or permanent relocation to a different town, state, country, or continent, menopause or andropause, a public health epidemiologic event, such as the changes in the incidence, prevalence or surveillance of a disease, such as Human Immunodeficiency Virus (HIV), Malaria, West Nile Virus (WNV), Cholera, Tuberculosis, diarrheal diseases, Small Pox, or Severe Acute Respiratory Syndrome (SARS), or such as an earthquake, flood, acts of terrorism or war, social or political unrest or other life event, community-level event, societal-level event or species-level event. The genetic testing analysis and reporting of results may be based on the initial DNA sample received from the individual, or a new DNA sample received at some later time, and may be based on the raw or already analyzed genetic testing data obtained from the initial genetic testing or from raw or already analyzed genetic testing data obtained from the individual since that initial time. This reporting can be either automatic, such as being notified automatically by e-mail, written report, in-person, telephone, facsimile, text message, webpage, or web conference or manual, such as if the individual must do something in order to access the analysis and results, such as accessing a specific website or calling a number, visiting an office, or contacting a third party in order to receive the analysis and results. Milestones that trigger reporting of initial analysis results or reflex testing analysis results, or both, is applicable to all species, including humans and non-humans, such as livestock and pets.

Reflex testing may be performed for individuals that are human as well as non-humans. Individuals may be human as well as other mammals (Mammalia) or Aves or Fish or Reptilia or other eukaryotes (such as Fungus or Protists) or prokaryotes (such as Bacteria and Archaea) or virus (including retroviruses and bacteriophage), including, but not limited to pets, such as dogs, cats, and birds; farm animals such as pigs, cattle or cows, goats, chickens, ducks, turkey, and sheep, as well as other animals, such as apes, bison, camels, horses (for example, racehorses, such as Harness and Thoroughbred), whales and dolphins. Genetic profiles may also be generated for plants, including but not limited to commercially important plants such as, for example, agricultural plants including but not limited to cotton plants, olive trees, evergreen coniferous trees, banana trees, apple trees, orange trees, grapefruit trees, cherry trees, almond trees, wheat, corn, hemp, soybeans and rice. Genetic profiles can be generated for fish, including but not limited to salmon, tuna, sea bass, Alaska pollock, cod, eels, tilapia, flashlight fish, anglerfish, Kryptophanaron alfredi, or sharks. Genetic profiles can also be generated for invertebrates, such as lobsters, shrimp, scallops, Tomopteris and insects; microorganisms, such as bacteria or viruses; and endangered species or extinct species from which genetic material can be obtained.

For example, phenotypes that may be tested for in non-human animal's may be coat color(s), eye color, nose color, size, temperament, intelligence, agility, speed, racing performance, performance at conformation events, amount of shedding, amount of milk production, percentage of protein in milk, percentage of fat in milk, muscle strength, amount of lean meat, height, weight, eye color, longevity, reproductive capacity, and diseases and disease susceptibility, such as hip dysplasia, exercise-induced collapse or colic. Initial and reflex phenotypes may be determined based on an agricultural company's, government's, farmer's, animal trainer's, veterinarian's, or pet owner's (or prospective pet owner's) preference. For example, a prospective pet owner may value a dog's grown size or aggressiveness first, and thus have an initial phenotype for grown size, aggressiveness or both. If, for example, the predisposition for a puppy's grown size fits the prospective pet owner's size restriction, reflex testing to the prospective pet owner's second criteria, such as intelligence or aggressiveness, is performed. If the puppy does not fit the prospective owner's size restriction, no reflex testing may be performed. Additional rounds of reflex testing may be performed.

Reflex testing can apply to both actual testing of the genotype (e.g., laboratory genetic test), or the analysis of the genotype, and/or the reporting of the genotype or phenotype or both. Reflex testing may also apply to only genotype testing and analysis or to only reporting of the genotype or phenotype or both. For example, reflex testing may mean that actual testing (genotyping) for those genetic variants is not conducted until a risk or predisposition or carrier status or diagnosis for the first phenotype, such as a disease or trait or process, is genotyped or before the risk or predisposition or carrier status or diagnosis for the first phenotype, such as a disease, is ascertained. Reflex testing may also mean that genotyping for the reflex phenotype, such as a disease, condition, trait or process, occurs either before or after or at the same time of the genotyping for the first disease or trait or process but the results are not reported (such as not entered into any genetic analysis algorithms or analyzed or being shown anywhere in the report for an individual's genetic profile or conveyed in any manner to the individual or entity that ordered the genetic profile, or who views or has access to the results) unless there is an increased or decreased predisposition or carrier status identified for the first phenotype. Reflex testing also applies to the physical testing and genotyping process, the analysis of the genotypes and phenotypes as well as using or conveying the results (whether genotypes or phenotypes or predisposition or carrier status or diagnosis or any or all of the above) by electronic means, by paper, in-person, by verbal means, or any other means, to the entity, person, information technology system, or analytical program that is conducting the testing or analysis, or both, as well as the person that ordered the test, views or has access to the test, as well as using the genotypes or phenotypes or predispositions for or in any analysis or interpretation of the raw or analyzed genotype data or any other genotypes or phenotypes or predispositions. This means that reflex testing is not time dependent upon when the initial genetic testing (the actual genotyping) is conducted and is also not dependent upon when the initial phenotype or panel's first round (before any reflex testing) of analysis for risk or predisposition or carrier status is conducted. Reflex testing may occur immediately following the diagnosis of a phenotype or the genetic testing (the actual genotyping), the initial analysis for the phenotype or panel or both (before any reflex testing is conducted), or at any other time point in the future, such as seconds, minutes, hours, days, weeks, months, years, or decades after either the initial genetic testing (the actual genotyping) or the initial analysis or both is conducted. Therefore, an individual may find that they are either genetically predisposed to coronary artery disease or have been diagnosed with coronary artery disease at earlier Time A, but then at a later time, Time B, either the individual, their healthcare provider, such as an Internist or Cardiologist or Pharmacist, or a third party, may want to analyze, deduce, investigate, ascertain or find out the individual's genetic risk or predisposition to adverse reactions to HMG-CoA reductase inhibitors (Statins). If genetic testing (actual genotyping) or genetic analysis (of genotypic data) or both is then conducted to ascertain the individual's risk or predisposition to adverse reactions from HMG-CoA reductase inhibitor medication at the later Time B, because it was known from earlier Time A that the person was predisposed to coronary artery disease or because the person was diagnosed with coronary artery disease, or both, then this also constitutes reflex testing. As stated, reflex testing may occur immediately, or any time in the future, such as seconds, minutes, hours, days, weeks, months, years or decades after one or more of the following: the initial genetic testing (actual genotyping) is conducted or diagnosis of the phenotype is made or predisposition or risk or carrier status for the phenotype is ascertained (through genetic analysis of the genotypic data).

Reflex testing can also be accomplished either on the front end or back end of the analytical or reporting process or both. For example, if an individual has an increased predisposition for obesity, or has a high body mass index, then the reflex may analyze or show the person's predisposition to diabetes mellitus, type II (diabetes). Reflex testing may work by the analytical process identifying that an increased predisposition for obesity is present (predisposition can either be increased chance of getting the phenotype or decreased chance of getting the phenotype or being a carrier of the phenotype, which means that the person either carries or has or likely has the phenotype) and therefore reflexes to showing predisposition for diabetes. Diabetes predisposition may only be shown if a person is found to be at increased or decreased predisposition or a carrier for obesity. If the person is not found to be at increased or decreased predisposition or a carrier for obesity, then diabetes predisposition may not appear in the report for the individual's genetic profile. This constitutes ‘front-end’ reflex. Alternatively, reflex testing could occur by the analytical process identifying (i.e. calculating from the alleles or genotype(s) of one or more genetic variants) both predisposition for obesity (either increased or decreased predisposition or carrier status) and predisposition for diabetes (either increased or decreased predisposition). If there is an increased or decreased predisposition or carrier for obesity then no change is made and the predisposition for diabetes is included in the analysis and is included in the genetic report. However, if no increased or decreased predisposition or carrier for obesity is found then the predisposition for diabetes is covered up (grayed out; blacked out; ignored; deleted; not shown, reported, or provided; made to appear less relevant or irrelevant; or such) and is either not displayed further in the analysis or the genetic report or both or is moved to the back of the genetic report, or otherwise made less relevant or irrelevant in the genetic reporting process, such as by putting it in a separate section of the report or conveying those results in a less relevant manner to the individual, such as by placing that information in a less relevant section of the genetic report, such as in a less relevant section of a webpage or website (for example, not placing the reflex phenotype information in the main or primary or same section where risk or predisposition or carrier status or diagnosis pertaining to the initial phenotype or the relevant phenotypes or the phenotypes found associated with either higher or lower risk is presented). This constitutes ‘back-end’ reflex testing.

In some embodiments, reflex testing is based on a predisposition or risk to a phenotype (e.g., disease, disorder, trait or condition). However, in some embodiments, reflex testing is not based on predisposition as some genetic variants are deterministic of disease, therefore reflex testing can be predicated upon an individual having a single genetic variant that is either deterministic for a phenotype (the individual either is a carrier but not affected by the phenotype or has or likely has the phenotype) or is associated with either increased or decreased predisposition for a phenotype. For instance, an individual may be found to carry a genetic variant that causes (is deterministic for) cystic fibrosis. If this occurs, then reflex testing may occur that will look at other genetic variants in order to ascertain degree of lung disease with cystic fibrosis, severity of cystic fibrosis and prognosis with cystic fibrosis.

In some embodiments, reflex testing is based on a phenotype that is not determined by genotyping or genetic analysis. For example, a medical history, or a diagnosis may indicate a phenotype such as for example cancer, or obesity or any of the phenotypes provided herein. The indicated phenotype may then cause another phenotype to be genotyped or not genotyped, to be analyzed or not to be analyzed, to be included in the report or not to be included in the report, to be included in a specific section of the report or not to be included in a specific section of the report, or any combination thereof.

The reflex phenotype may or may not be included in the raw data or as part of the preliminary analysis but its inclusion in the near-final and final report that is delivered to the individual, the health-care provider, or any third-party who ordered the test is determined by whether or not there is a diagnosis, carrier status or an increased or decreased predisposition of the first phenotype, or whether a specific milestone event (trigger event) has occurred (as discussed previously). As reflex testing can go through multiple rounds and multiple layers deep (for example, first phenotype reflexes to second phenotype that reflexes to third phenotype that reflexes to fourth phenotype, etc.), this is applicable to each and every step. For instance, if reflex testing is indicated (either by one or more deterministic genetic variants (carrier) or by an increased or decreased predisposition for or a diagnosis of a phenotype) for the second phenotype, which is also found to be increased risk and this causes reflex testing for a third phenotype. As an example, if a person is found to be predisposed to obesity then reflex testing may occur to round 2 to discern the person's predisposition for diabetes and if the person is predisposed to diabetes then reflex testing may continue on to round 3 to discern the age of onset of diabetes and if the person has a predisposition for greater or less effectiveness of or adverse reactions to any medications that are used to treat pre-diabetes or diabetes. Reflex testing examples are shown in FIG. 13.

Reflex testing of the second phenotype may cause the analysis or reporting of the first phenotype to be modified. For instance, if a deterministic genetic variant for Hemochromatosis is found and reflex testing shows that other genetic variants indicate that Hemochromatosis may be severe, then the report may indicate this (that the person has a genetic variant that is associated with Hemochromatosis and that the disease presentation may be severe). The reflex testing may also cause both phenotypes to not be reported. An increased predisposition for a disease may be ascertained based on the allele or genotype of one or more genetic variants. This may cause reflex testing of an associated phenotype that negates the first phenotype. For example, the disease Hemochromatosis may be found initially but reflex testing may examine and analyze other genetic variants that may be associated with either very low or no penetrance or expressivity of Hemochromatosis for that individual. Therefore, neither Hemochromatosis nor the reflex testing results, or both, may be included anywhere in the analysis or Genetic Report or, alternatively, they may both be included in the report, such as in the main section or in a different section. Both phenotypes also may both be included in the raw analytic data, one may be included in the raw analytic data, or neither of the phenotypes may be included in the raw analytic data.

Reflex testing may take into account many different factors besides the genotype of one or more genetic variants. These non-genotype factors (such as lifestyle or request or diagnosis) may either occur at the first step (as in the example where the person is a smoker and this causes reflex testing to lung cancer risk) or at a later step (such as where risk of uterine cancer is deduced but the reflex to medical history shows the person had a hysterectomy and therefore the risk of uterine cancer is not included in the analysis of the organ system or in the analysis of the entire genetic health of the individual and may or may not be included in report and in any correspondence with the ordering person or entity or third party or the person the genetic material was from).

The risk for the reflex phenotype may be tested at the same time as the initial phenotype; for example, a single sample may be used to test both the initial phenotype and the reflex phenotype. Alternatively, the reflex phenotype may be tested after the initial phenotype, and another sample used, or perhaps an aliquot of the initial sample that was stored may be used. The reflex phenotype and the initial phenotype may be tested at the same time, and the results for each test may be analyzed at the same time. Alternatively, the reflex phenotype and the initial phenotype may be tested at the same time, and the results analyzed at different times. For example, if the initial phenotype produces a positive result, the results for the reflex phenotype may be then analyzed. The reflex phenotype can be reported concurrently with the initial phenotype, or subsequent to the initial phenotype. The reflex phenotype can be initially requested by the individual, or third party, or after the individual receives the results of the initial phenotype, and optionally, after consultation with a genetic counselor, physician, nurse practitioner, physician assistant, other healthcare provider, or third party. The reflex phenotype(s) may cost additional fees.

The panels described herein are used for determining the risk or predisposition of at least 2 phenotypes, which may include 2 phenotypes in the initial round of analysis or 1 phenotype in the initial round of analysis and 1 or more phenotypes deduced via reflex testing. The phenotypes may be monogenic, multigenic, or multifactorial and each phenotype may be associated with one or more of the following: monogenic, polygenic, or multifactorial genetic variant(s). The panels can be used for determining the risk or predisposition of 1, 2, 3, 4, 5 or more multifactorial phenotypes alone or 1, 2, 3, 4, 5 or more monogenic phenotypes alone or both 1, 2, 3, 4, 5 or more multifactorial and 1, 2, 3, 4, 5 or more monogenic phenotypes. The multifactorial and monogenic phenotypes can be tested for and analyzed together, either at the time the initial genetic testing is conducted or at any other time based on genetic testing data (the detection of genetic variants via arrays, microarrays, massarrays, beadarrays, PCR, from partial or full exome or partial or full genome sequencing, such as with nanopore sequencing, or any other methodology that allows for the detection or identification of genetic variants throughout a genome).

A panel can be premade and presented to the individual, entity or third party ordering the genetic testing or genetic analysis or both, or the panel can be chosen at the time of consultation from a list of phenotypes (such as the phenotypes as shown in FIG. 15-18) that may be grouped according to organ system, disease process, age of onset, clinical relevancy, lifestyle relevancy or any other grouping as portrayed in FIG. 15-18. The list of phenotypes may appear on a laboratory requisition form. The list may be in alphabetical order or grouped according to organ system, medical specialty, disease process, age of onset, clinical relevancy, lifestyle relevancy or any other grouping as portrayed in FIG. 15-18. An individual, entity, or third party ordering the genetic testing or genetic analysis or both may then choose a subset of these phenotypes such that a panel is constituted by a group of 2, 3, 4, 5, 6, 7, 8, 9, 10 or more phenotypes, or up to 10, 20, 25, 30, 35, 40, 45, 50, 100, 200, 300, 400, 500, or 1000 phenotypes. Individuals may also choose other options, such as shown in FIG. 20. These phenotypes may then either be tested for or, if the raw genetic data already exists, then the genetic analysis may be conducted and all applicable reflex testing conducted for these phenotypes, taking into account each of the phenotypes selected both individually and in-relation to each other, and a genetic report can be produced.

The panels (also referred to as genetic testing panel or a genetic panel, and other variations thereof) can be defined as any group of two or more phenotypes reported together at any time, regardless of when the genetic testing (the actual genotyping) occurred. For example, a fetus or newborn may undergo full genome sequencing so that in part or substantially the entire genetic code is obtained at that time. The phenotype information that is then analyzed or conveyed in a report, or medical record, can constitute a panel. The analysis, reporting or both, of the phenotypes in regards to being designated a panel is not time-dependent in relation to when the genetic testing (genotyping) occurred. For example, a newborn has full genome sequencing but the individual newborn later on had their risk or predisposition for at least 2 phenotypes on the Pediatric Panel Beta determined when they are 10 years old, the phenotypes determined constitute a panel. In another example, an individual with full genome sequencing as a newborn is tested and analyzed by the methods of the present invention. The phenotypes analyzed or reported on exist within or are from the Full Genome Panel Alpha, Beta or both. The analysis, reporting, or both, can occur at any time, for example, during the initial genotyping (such as sequencing) or at any later date, such as seconds, minutes, hours, days, weeks, months, years or decades later, and the analysis, reporting or both of the phenotypes at any point of time still constitutes a panel. For example, the phenotype(s) may be determined initially, at approximately the same timeframe as the obtaining of genetic data, or may be determined later, or a combination thereof, such that some phenotypes are determined initially and some phenotypes are determined at a later time.

Any genetic variant-phenotype associations or phenotypes based on genetic information that is analyzed or reported together as a group, or both, constitutes a panel. This allows for genetic sequence information containing genetic variant information to be stored in a database or other device or medical record and to be analyzed either at that time or at a later date, such as when the information, such as clinical information, may be more pertinent or useful to a medical professional, or both. Panels and/or reflex testing and/or OP-CADI may also be ordered by and are applicable to patients, clinicians, veterinarian or veterinary surgeon, pet owners, animal owners, pharmacists, healthcare providers, insurance companies, hospitals, clinics, academic researchers, laboratory researchers, clinical researchers, pharmaceutical companies, agricultural companies, agricultural managers, ranchers, farmers, military personnel, governmental agencies, local, national and international agencies, such as the United States Food and Drug Administration (FDA), European Union (EU), United States Centers for Disease Control (CDC), United Nation's World Health Organization (WHO), World Organisation for Animal Health (OIE), United Nation's Food and the Agriculture Organization (FAO), or any entity that may be interested in or able to utilize genetic information. For example, specific panels can be as grouped as in FIG. 15-18, or variations thereof.

The panels described herein, and subsets thereof, may be used for a variety of applications and in a wide range of settings. Similarly, a wide range of persons may request a genetic test. For example, the individual to be tested, an individual seeking to confirm paternity, a pregnant woman seeking information about her current or future fetus (current or future baby), the parent or guardian of a minor to be tested, an individual or couple seeking information about potential sperm or egg donors or about the actual sperm or egg or embryo itself (such as Carrier Screening Panel, Embryo and Fetus Panel Alpha, Embryo and Fetus Panel Beta, Female Fertility Panel, Male Fertility & Erectile Function Panel, Pregnancy Panel, Assisted Reproductive Technology Panel, Reproduction, Egg & Sperm Donor Screening Panel Alpha, Reproduction, or Egg & Sperm Donor Screening Panel Beta), a medical professional, a medical specialist, a therapist, a pharmacist, a weight loss specialist, a counselor, an athletic trainer, an athletic coach, a fitness advisor (such as Exercise, Fitness and Athletic Training Panel, Dietary, Nutrition & Weight Management Panel Alpha, Dietary, Nutrition & Weight Management Panel Beta), a representative of or member of or part of the local, state, or federal government such as the military or armed forces (for example, Military and Armed Forces Panel Alpha (FIG. 16), Military and Armed Forces Panel Beta (FIG. 17)), or first responders, emergency medical services, the police (for example, Law Enforcement/Forensic/Investigative Panel (FIG. 18), Emergency Panel), or other governmental agency or subagencies or related agencies such as the Secret Service, the Department of Defense (DoD), the Defense Advanced Research Projects Agency (DARPA), Department of Homeland Security (DHS), the Federal Bureau of Investigation (FBI), the National Security Agency (NSA), the Bureau of Alcohol, Tobacco, Firearms and Explosives (ATF), the Central Intelligence Agency (CIA), the National Reconnaissance Office (NRO), the Joint Special Operations Command (JSOC), the Defense Intelligence Agency (DIA), the Bureau of Intelligence and Research (INR), the Office of Intelligence and Counterintelligence, the Drug Enforcement Administration (DEA), National Aeronautics and Space Administration (NASA), or international agencies such as North Atlantic Treaty Organization (NATO), the United Nations (UN) and the UN Security Council, or any other government or governmental agency of any country or collaboration of countries, such as the Secret Intelligence Service (SIS) and MI6, the Defense Intelligence Staff (DIS), the HaMossad leModi'in uleTafkidim Meyuhadim (Mossad), the Canadian Security Intelligence Service (CSIS), the Bundesnachrichtendienst (BND), the Naikaku Jöhö Chösashitsu (Naichö), the Militaire Inlichtingen-en Veiligheidsdienst (MIVD), the Nasjonal sikkerhetsmyndighet (NSM), the Inter-Services Intelligence (ISI), the Federalnaya Sluzhba Bezopasnosti (FSB), the Re'asat Al Istikhabarat Al A'amah (GIP), the Security and Intelligence Division (SID), the Indian Space Research Organisation (ISRO), the National Directorate of Security (NDS), the Centro Nacional de Inteligencia (CNI), the National Security Bureau (NSB), the Directorate-General for External Security, the National Intelligence Service (NIS), or any other governmental organization or agency, as well as aerospace, defense, or advanced technology companies such as Lockheed Martin, Raytheon Company, or Northrop Grumman Corporation, or private security companies or private military companies (PMCs), such as Blackwater Worldwide, ArmorGroup International PLC, Hart Security, Military Professional Resources Inc. (MPRI), or Pacific Architects and Engineers, or other third party, as well as an employer or potential employer, an insurance company (for example, Insurance Panel Alpha, Insurance Panel Beta), or other third party. These panels may also be useful to applicants or participants of programs or agencies that require one or more of the following: security, secrecy, physical conditioning, training, aptitude, ability, base requirements or psychological conditioning, training, exceptional aptitude, exceptional ability, or base requirements, such as a space program, such as applicants to, employees of, consultants to, members of, or individuals associated with private space flight, such as Virgin Galactic, Benson Space Company, EADS Astrium, Rocketplane Limited, Inc., Space Adventures, XCOR Aerospace, Arianespace, S.P. Korolev Rocket and Space Corporation Energia, Launch Services Alliance, United Launch Alliance, Bigelow Aerospace, or SpaceDev, or governmental space programs, such as the National Aeronautics and Space Administration (NASA), the European Space Agency (ESA), the Federal'noe kosmicheskoe agentstvo Rossii (Roskosmos), the Dokuritsu-gyösei-hōjin Uchū Kōkū Kenkyū Kaihatsu Kikō (JAXA), the Sohnut HaHalal HaYisraelit (ISA), the Natsional'ne kosmichne ahentstvo Ukrayiny (NSAU), the Bhāratīya Antariksh Anusandhān Sangatn (ISRO), and the China National Space Administration (CNSA).

An individual who has received certain results from a medical examination or medical test may also be tested with a specific panel, or subset thereof. One or more panels, or subsets thereof, may be selected based on the medications or supplements (e.g., vitamins, herbal supplements, minerals) an individual is taking or considering taking or may take in their future. A panel, or subset thereof, may also be used to test an individual who leads, or has led, a particular lifestyle(s) or who possesses specific phenotypes, such as trait(s), or wants to find out if they or their current or future children have or may have specific phenotypes, such as traits. A panel, or subset thereof, may also be used to test an individual who is applying for school, employment, military or armed forces service, insurance (health, life, liability, disability, employment, work, or any other type of insurance), or who is being considered by a third party (such as a program, school, college, university, athletic event, sports team, potential or current employer, government employment, the military, an insurance company) for any of the above. Individuals interested in one or more specific panels, or be directed to submitting samples for specific panels, may have one or more specific indications, such as, but not limited to, those listed in FIG. 21. In FIG. 21, the items designated by an asterisk and bold type have a particularly high association with a phenotype or indicated panel.

Individuals that may be interested in the panels may be those interested in their future offspring's genetic profile and phenotypes. Such individuals can have a genetic profile determined from the combined analysis of the offspring's prospective parents. The method is referred herein as “offspring projections from the combined analysis of different individuals”, or OP-CADI (see FIGS. 14, 20). The genetic profile of each of the individual's parents is first individually analyzed and then combined. Thus, provided herein is a method of utilizing the genotypic and phenotypic information from these two individuals to view potential genetic profiles of their potential future offspring by comparing phenotypes, genes, loci, genetic variants, as well as carrier status and the odds ratios or other risk values attributed to each genetic variant, in order to ascertain the future offspring's lifetime risk ranges for the phenotypes and carrier status of phenotypes. OP-CADI can be applicable to a single genetic variant, a single gene, a single locus, a single phenotype, part of the genome or the entire genome and may take into account monogenic, polygenic, and/or multifactorial phenotypes, as well as potential or suspected environmental factors that the offspring may be exposed to or interact with.

For example, the female's genetic profile may be found to have genetic variants associated with Epidermolysis Bullosa Simplex, Cystic Fibrosis, Alzheimer's Disease, Macular Degeneration and being an Endurance Athlete while the male's genetic profile may be found to have genetic variants associated with Prostate Cancer, Cystic Fibrosis, Androgenic Alopecia, Alzheimer's Disease, and being an Endurance Athlete. The future child's genetic profile and analysis may contain the following information: Epidermolysis Bullosa Simplex—25% chance of being a carrier, 75% chance of being a non-carrier, Cystic Fibrosis—25% chance of being affected, 50% chance of being a carrier, 25% chance of being a non-carrier (neither affected nor a carrier), Alzheimer's Disease—Predictive Medicine Lifetime Risk Range 23-45%. Macular Degeneration—Predictive Medicine Lifetime Risk Range 15-25%. Androgenic Alopecia—Predictive Medicine Lifetime Risk Range 5-25%, Prostate Cancer—Predictive Medicine Lifetime Risk Range 14-22%, Endurance Athlete—Very high probability of having this trait, such as greater than 75% chance.

Couples interested in having children using their own genetic material, or by assisted reproductive technologies, such as by showing the potential genetic profile of offspring from an egg donor or sperm donor or both, may use OP-CADI. Breeders of lifestock and other animals, animal researchers, and individuals, researchers, or companies working with animals, mammals, fish, birds, reptiles or plants may also utilize OP-CADI in order to ascertain the projected phenotypes from different mate pairings, and they may base their mate selection on results that show either an increased likelihood of one or more phenotypes or a decreased likelihood of one or more phenotypes, or an increased likelihood of one or more genetic variants or genes or loci or a decreased likelihood of one or more genetic variants or genes or loci, and/or the carrier status of one or more phenotypes, from the OP-CADI results from one or more mate pairings analyzed. OP-CADI may also allow for genetic information from genetic testing on a number (from 1 to over 1,000,000,000) of both potential male and female parents to be analyzed together and to create mate pairs that are more likely to produce one or more genotypes and/or phenotypes, or that are less likely to produce one or more genotypes and/or phenotypes, or a combination of the two (some genotypes and/or phenotypes are more likely while other genotypes and/or phenotypes are less likely, with one or more of the following: monogenic, polygenic, or multifactorial phenotypes). OP-CADI is applicable to all species, including human and non-human, that produce offspring through the combination of genetic material from two parents. Also provided herein are methods for matchmakers and matchmaking services to use this method for matching individuals based on their genetic profiles or the genetic profiles of their potential children or both.

The OP-CADI takes into account the fact that offspring inherits approximately 50% of their autosomal genetic code from one parent and 50% from the other parent. At the same time, male children typically have a 100% chance of inheriting their y-chromosome from their father and females typically have a 100% of inheriting the one X-chromosome that the father has. At the same time, both male and female children typically have a 100% chance of inheriting the mitochondrial genetic code from their mother.

By analyzing the respective genetic codes of both parents, projections can be made about the potential genetic code and the potential phenotypes of their offspring. Monogenic diseases follow Mendelian inheritance patterns (see for example, FIG. 2), and penetrance and expressivity may be determined through published data on the specific genetic variant(s), the gene, or the phenotype, such as the disease, and through an analysis of the genetic sequence and genetic variants that the parents' genomes contain. Thus, the chance of a child being affected with a phenotype, being a carrier of a phenotype, or being neither affected nor a carrier (a non-carrier), also known as their carrier status, as well as being at increased risk for a phenotype or decreased risk for a phenotype, can be deduced and this information can then be supplied to the individual interested in having the potential offspring or their physician or veterinarian or veterinary surgeon or agricultural manager or agricultural company or rancher or farmer or other third party. Polygenic and multifactorial disease risk is determined by calculating the AS, CAS, CGR or PMR (as described herein), but OP-CADI combines both the potential father and potential mother's genetic profile in order to project or predict the genotypes and phenotypes of their potential offspring. The father most often contributes ˜50% of his autosomal genetic code, 100% of his y-chromosome code to any sons and 100% of his x-chromosome code to any daughters while the mother most often contributes ˜50% of her autosomal genetic code, one of her two X-chromosomes to any daughters, and 100% of her mitochondrial DNA to their offspring.

For the autosomal genetic code, in some embodiments, the first step in the analytical process is for a manual operator or the genetic analysis information technology system to perform multiple ‘chops’, with each chop taking into consideration approximately 50% of all the genetic variants that make the cut-off (such as GVDC ≧1.5) for each phenotype being accessed (determined by the genetic variant(s), gene(s), locus, or phenotype(s) or panel(s) chosen for analysis) in order to determine the lowest possible lifetime risk and the highest possible lifetime risk for each polygenic or multifactorial phenotype. Therefore, OP-CADI analyzes all relevant genetic variants throughout the entire genome (that make the cut-off) in-relation to each phenotype. This is done separately for the female parent and for the male parent. The genetic profile chop for the female parent containing the genetic variants that constitute the lowest lifetime risk for each phenotype being assessed is designated “Mother—Low” and the genetic profile chop for the female parent containing the genetic variants that constitute the highest lifetime risk for each phenotype being assessed is designated “Mother—High”. The genetic profile chop for the male parent containing the genetic variants that constitute the lowest lifetime risk for each phenotype being assessed is designated “Father—Low” and the genetic profile chop for the male parent containing the genetic variants that constitute the highest lifetime risk for each phenotype being assessed is designated “Father—High”. Monogenic disorders generally follow monogenic Mendelian inheritance patterns for genes and genetic variants located on autosomes. Once the set of genetic variants that constitute the lowest lifetime risk and the highest lifetime risk for each phenotype being accessed has been deduced, these final-chop profiles from the female and male are merged, as described below.

Genetic variants that exist close to each other on the same chromosome are more likely to be inherited together, and therefore different chops are also made taking into account different measures of linkage disequilibrium, such as a chop when the r² 0.5, r² 0.75, or r²≧0.90 in order to discern the changes in the risk values (such as the predictive risk values) obtained if these genetic variants are inherited together or if they are not, along with the chances that they will be inherited together or that they won't be inherited together based on their r² or D values. All genetic variants with a r²≧0.99 may be analyzed as being inherited together and may not be separated (separated meaning that the two genetic variants may be analyzed as potentially being separated during the inheritance process simulated by each chop, such as for example that one genetic variant is inherited while the other genetic variant is not) during the chop process. Alternatively, it may be designated that all genetic variants with a r²≧0.95 or any operator-designated r² cut off value may be chosen so that any two or more genetic variants with an r² below that threshold may be separated during the chop process and any two or more genetic variants with r² above the designated threshold will be analyzed as being inherited together (meaning that they may not be separated during the chop process).

For the sex chromosomes (for example, the X- and Y-chromosomes in Homo sapiens) genetic code, for the female parent, 50% of all the genetic variants from her X-chromosomes for each phenotype being assessed from her are analyzed within each chop. The genetic variants that constitute the lowest lifetime risk for each polygenic or multifactorial phenotype is combined within “Mother—Low” genetic profile and the genetic variants from the chops that constitute the highest lifetime risk for each phenotype are combined within “Mother—High”. This is applicable to both the potential female and male offspring. For the male parent, approximately 100% of the genetic variants from his single X-chromosome is combined into “Child—Low” and also “Child—High” for the potential female children. For potential male children, approximately 100% of the genetic variants from his (the male parent's) single Y-chromosome are combined into “Child—Low” and also “Child—High”. Monogenic phenotypes, such as disorders, may follow monogenic Mendelian inheritance patterns or sex-linked inheritance patterns for genes and genetic variants located on sex chromosomes. For species other than human, known species-specific inheritance patterns for each chromosome from each parent can be utilized in a similar way in order to conduct the OP-CADI for any species where the genetic material of the offspring is from the combination of genetic material from two parent organisms.

For the mitochondrial genetic code, all offspring (e.g. children) are expected to inherit 100% of the mitochondrial genetic code from the female parent. Because of this, the analysis of the mitochondrial genetic code for “Child—Low” and “Child—High” (described below) utilizes 100% of the mitochondrial genetic code from the female parent. Sometimes genetic variants from the autosomes or sex chromosomes or both are analyzed together with mitochondrial genetic variants in the determination of carrier status or risk of certain phenotypes (for instance, in the analysis of the ‘Exercise Intolerance’ as well as the ‘Deafness’ phenotypes). In this case, the OP-CADI analysis takes into consideration all of the female parent's mitochondrial genetic variants for those phenotypes being assessed and ignores the male parent's mitochondrial genetic variants.

This merger involves two parts, as shown in FIG. 14. Part 1 involves taking the genetic variants that constitute the lowest lifetime risk for a phenotype from the female parent (Mother—Low) and combining them with the genetic variants that constitute the lowest lifetime risk for a phenotype from the male parent (Father—Low). This new genetic profile is designated “Child—Low” and all the genetic variants (now a combination containing approximately 50% from the male parent and approximately 50% from the female parent) are run again through the genetic analysis system in order to arrive at the lowest lifetime risk value possibility for the possible offspring of these two parents. Part 2 involves taking the genetic variants that constitute the highest lifetime risk for a phenotype from the female parent (Mother—High) and combining them with the genetic variants that constitute the highest lifetime risk for a phenotype from the male parent (Father—High). This new genetic profile is designated “Child—High” and all the genetic variants (now a combination containing approximately 50% from the male parent and approximately 50% from the female parent) are run again through the genetic analysis system in order to arrive at the highest lifetime risk value possibility for the possible offspring of these two parents. Combining the lifetime risk values ascertained for Child—Low and the Child—High gives a range, with the lowest value possibility being the value for Child—Low and the highest value possibility being the value for Child—High. This constitutes the lowest lifetime risk and carrier status and highest lifetime risk possibilities and carrier status (the “Child—Range”) for one or more phenotypes for any potential offspring.

Taking into account the fact that X-inactivation will typically occur in any mammalian female offspring, the OP-CADI gives the range of possibilities. The potential genetic profiles of mammalian female children (Child—Low and Child—High) primarily looks at a mosaicism pattern and therefore considers the two X-chromosomes (the one paternally derived X-chromosome and one of the X-chromosomes from the female parent, as well as repeating the steps of the analysis, this time with the same paternally derived X-chromosome but now with the other maternally derived X-chromosome) no different from the autosomes in the analysis in terms of finding the lowest range value and highest range value and the most likely outcome existing within this range. In some embodiments, this can be accomplished by assessing the phenotypes if the X-chromosome from their father is inactivated and then another assessment where the X-chromosome from their mother is inactivated. However, the mosaicism pattern created due to lyonization is also taken into account by determining the carrier status and risks of phenotypes when one X-chromosome is inactivated versus when the other X-chromosome is inactivated, and these different chops of examining and predicting lyonization results, may provide for a different, and potentially more accurate range for the potential female children. Similarly, the potential for crossing-over between the paternally derived X-chromosome and the maternally derived X-chromosomes' pseudoautosomal regions may be considered when assessing phenotypes associated with one or more genetic variants on one or more sex chromosomes. Inheritance laws surrounding X-inactivation may be species specific and are known to persons of ordinary skill in the genetic analysis arts, and are integrated into the OP-CADI algorithm. For example, X-inactivation in marsupials occurs only to the paternally derived X-chromosome and therefore marsupial OP-CADI will analyze the potential offspring with the paternally derived X-chromosome inactivated (and the maternally derived X-chromosome will not be inactivated), meaning that the genetic variants and their associated phenotypes on the paternally derived X-chromosome may not affect the offspring and may not appear in the analysis and/or the report.

The OP-CADI can also give separate results for a potential female offspring (such as a child) and a potential male offspring (such as a child), and thus a separate OP-CADI Genetic Report can be generated for the potential female offspring (such as children) and the potential male offspring (such as children). The primary difference occurs with whether the male parent's X-chromosome is considered in the analysis (for all female children) or whether the male parent's Y-chromosome is considered (for all male children). The offspring are separated by gender, so that OP-CADI can give risk and carrier status information about the potential female offspring and about the potential male offspring. It may be that the male offspring has a significantly higher risk or has an affected carrier status of a harmful phenotype, such as if it is an x-linked disease, and thus the male offspring and female offspring's OP-CADI report may be different. Based on this information and analysis, mate pairing and possible sex selection methods can then be utilized, such as sperm sorting, pre-implantation genetic diagnosis and prenatal diagnosis, in order to choose or increase (or decrease) the likelihood of any phenotype(s), such as of gender, or of any genetic variant(s), gene(s), genetic sequence(s), or chromosome(s).

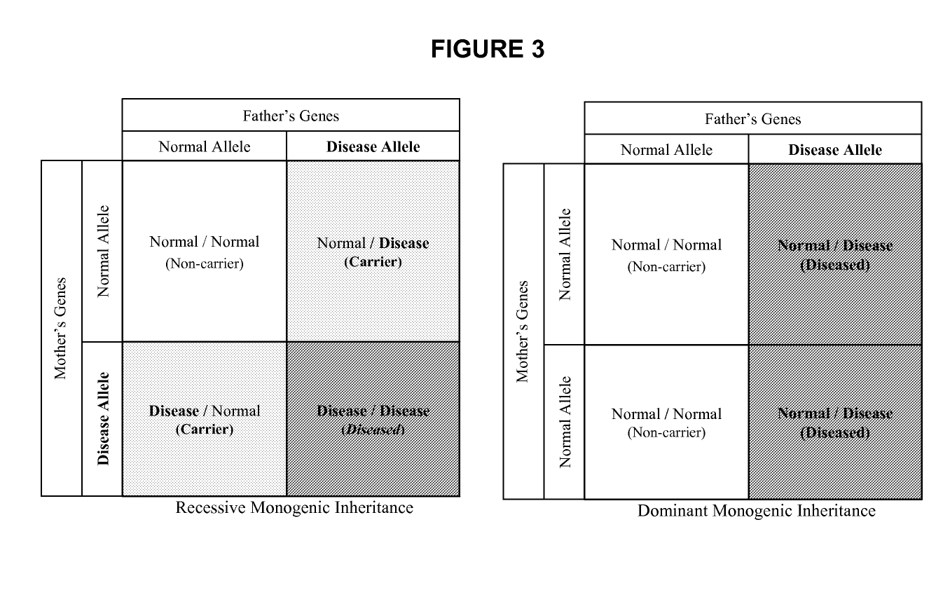

The above methodology can also be used for polygenic, multifactorial and/or monogenic phenotypes. For example, for monogenic phenotypes, probabilities of a phenotype may be given. For instance, if both parents are carriers of a genetic variant in the CFTR gene associated with the cystic fibrosis phenotype, then the probability of their child being affected with cystic fibrosis is 25%, the probability of the child being a carrier of a genetic variant associated with cystic fibrosis is 50%, and the probability of the child being neither affected nor a carrier (a non-carrier) is 25%. This type of probability deduction follows the tenants of monogenic Mendelian inheritance (see for example FIG. 3).

Multifactorial phenotypes (that take into interactions between one or more genetic variants and the environment), may be treated as polygenic. However, the OP-CADI Genetic Report for the offspring, such as children, may contain information relating to how environmental factors may influence the risk of certain phenotypes. For example, the child (or prospective child) may be found to have a low genetic lifetime risk of lung cancer if they do not smoke but a significantly increased lifetime risk of lung if they do smoke. The genetic profile may also find that the child has an increased risk for nicotine dependence and that they are more likely to start smoking at a younger age. By supplying this information to the parents, the parents can recognize how different environmental factors may influence their potential children. Alternatively, the OP-CADI may take into account environmental factors, such as if it is known that the offspring will live in an urban environment or if it is known that the offspring will be farm-raised, or raised for a specific function, such as equine raised for Thoroughbred or Harness racing. These environmental inputs for multifactorial phenotypes may then be utilized during the OP-CADI analysis so that the analysis and results may be interpreted with these non-genetic factors as well.

Genomic imprinting applies to certain phenotypes when the phenotypes only arise, or have a greater or lesser probability of arising, when the gene (containing the genetic variant(s)) is inherited from a specific parent (such if a phenotype only manifests if the genetic variant is inherited from the mother, while if it comes from the father than there is either a different phenotype or no discernable phenotype, and vice versa). Genomic imprinting, regarding phenotypes for which this is known to apply, is taken into account during the analysis process with the OP-CADI. For example, the diseases Prader-Willi Syndrome and Angelman Syndrome both are determined via parent-of-origin genomic imprinting. For genetic variants that are associated with these diseases, if the genetic variant comes from the female parent's genetic code then the probability of disease relates to Angelman Syndrome but if the genetic variant comes from the male parent's genetic code than the probability of disease relates to Prader-Willi Syndrome. Genomic imprinting relates not only to monogenic diseases but also to polygenic and multifactorial diseases. For example, atopy, atopic dermatitis and asthma have all been associated with genetic variants in the SPINK5 gene, but only when those genetic variants are maternally inherited. (Walley et al. Nat Genet 29(2): 175-178 (2001)). Probabilities and risk-ranges of some phenotypes depend on which parent is contributing the genetic variants (as ascertained from published literature) and this is taken into account with the OP-CADI.

The OP-CADI may also be used for parent selection (mate selection) purposes, such as when choosing either a female egg donor or male sperm donor or both. For example, if a married couple is unable to have children because the female has fertility issues, the couple may choose to search for an egg donor while planning to utilize the husband's sperm in order to fertilize the egg. The OP-CADI can be used as a scanning methodology that utilizes the husband's genetic profile and combines it with an egg donor's profile in order to assess the possible genotypes and phenotypes of the potential children. This process can be run for all possible egg donors under consideration, either one at a time, in batches of egg donors, such as 2 or 5 or 10 at once, or by utilizing the genotypic information available from all egg donors at once. If the genetic profile (genotype, such as for example via genechip analysis, PCR analysis, or sequencing) of the egg donors has already been deduced, then this process may be run automatically back-to-back or simultaneously until a certain genotype or phenotype probability or risk-range or carrier status is deduced. For example, the couple may want to ensure that the potential child will have the lowest risk-range of breast cancer possible, then the OP-CADI can be utilized to scan the available egg donor's genetic profiles in-order to ascertain which egg donor(s) will provide the lowest risk-range for breast cancer both on its own and when combined with the male parent's genetic profile. This approach can be utilized for any phenotype, and can be utilized for either just one phenotype or multiple phenotypes (for example, the lowest probability for all rare diseases and the lowest risk-range for breast cancer, Alzheimer's disease, and heart disease as well as the highest probability or highest risk-range for enhanced longevity, intelligence and blond hair). The above process can be utilized for any parent selection purpose that wants to take into account the genetic profile of the potential children. For example, it may also be utilized by a woman who wants to discern who the best sperm donor(s) will be based on certain cut-offs that they impose upon the potential future children's genetic profile (for example, less than (<)25% probability of any rare disease, metabolic disease, or syndrome).

A similar process may also be used by matchmakers or matchmaking services such that individuals submit their DNA or genetic profile and the matchmaker or service uses the OP-CADI to determine the potential genetic profiles for each match's potential children. Based on cut-off values supplied by either the matchmaking service for the individuals themselves (such as all matches must have less than (<) 25% probability of rare diseases), individuals can then be matched up. This information may also be combined with other analysis of each of the individuals own genetic profiles, for example to determine compatibility based on degree of sexual responsiveness. This constitutes a comprehensive analysis of all available genetic information in-order to try to ascertain the most appropriate or best matches on a genetic level set by certain cut-offs that are either determined by the matchmaker, the matchmaking service, or the individuals themselves. This may further be combined with each individual's personal preferences (such as preference for the other person's hair color or education level) in order to arrive at matches that are matched based on both genetic and personal preference factors.

The above process for the OP-CADI refers to genetic data ascertained through any method. For example, genetic data may be from array testing or nanopores or any other techniques that may not identify which specific chromosome that the genetic variant is from. For gene sequencing, full exome sequencing and full genome sequencing, each individual chromosome may be seen as a discrete entity. The information pertaining to which specific chromosome a genetic variant is contained on can be utilized within the analysis in order to identify a string (two or more) genetic variants that are likely to be inherited together as those genetic variants occur close to each other on the same chromosome. A string of genetic variants may represent a haplotype or multiple haplotypes or it may just represent two genetic variants that are within physical proximity to each other on the same chromosome. Groups of genetic variants that exist closer together on the same chromosome may then move with more frequency together during each chop analysis. The effects of crossing-over may be taken into account and integrated into the OP-CADI by selecting a certain distance (such as in kilobases, or in centimorgans) that is more likely to segregate together. Genetic variants that exist on the same chromosome and within that certain distance from each other will then most likely segregate together and may not be separated during the chop process.

The OP-CADI can also be applied to non-humans, such as with the breeding of Felis catus, Bos taurus, Gallus gallus, Pan troglodytes, Canis lupus familiaris, Capra hircus, Equus caballus, Mus musculus, Sus scrofa, Rattus norvegicus, Ovis aries, Meleagris gallopavo, as well as other non-human mammals, aves or fish or plants. For example, the OP-CADI can be used to detect the pairs of canines (such as Canis lupus familiaris) that are most likely to produce offspring that are faster runners, have enhanced nighttime eyesight or have specific coat color. For bovine (such as Bos Taurus), this novel approach can be used to detect the pairs that are most likely to have more offspring, or offspring that are greater in size or produce larger amounts of milk. For species other than human, known species-specific inheritance patterns for each chromosome from each parent can be utilized in order to conduct the OP-CADI for any species where the genetic material of the offspring is from the combination of genetic material from two parent organisms.

The same process of genetic analysis applies to the OP-CADI as before, including applying the OP-CADI to any genetic variant(s), gene(s), locus, phenotype(s) or panel(s) and incorporating the option for reflex testing, which allows for a comprehensive, dynamic analysis of genetic information. The OP-CADI can utilize and report on lifetime risk-ranges and probabilities of genotypes or phenotypes or both, it can also be utilized and report on action score risk-ranges and probabilities of phenotypes, and it can also utilize and report on cumulative action score-ranges or genetic health score ranges (such as existence score-ranges) and probabilities of phenotypes (such as for carrier status).

Any individual may be tested using one or more Full Genome Analysis Panel (such as shown in FIG. 15, or subset thereof) in order to determine his or her risk of, or predisposition for, one or more conditions, as shown in FIG. 15. A full genome analysis panel may aid the calculation of the general health of the individual or of a zygote, embryo or fetus. An individual with a family history of a specific condition (e.g., acute disease, chronic disease, degenerative disease, fatal disease) may be tested with a full genome analysis panel. An individual on a particular nutritional plan or diet may also be tested with such panel. In some cases, results from the Full Genome Analysis Panel(s) may prompt the individual to seek the advice of a physician or alternative professional, to make changes in his or her lifestyle (e.g., diet, exercise, smoking habit, caffeine intake, alcohol intake, drug use), or to take actions to mitigate the individual's risk of developing an adverse condition. A full genome analysis panel may also be run on a fetal genetic material (such as from a zygote, embryo or fetus) or on newborns or children in-order to analyze and assess their entire genetic genome including phenotypes that may negatively or positively affect their life.

As is the case with the other panels described herein, if an individual is diagnosed with or tests positive (either increased or decreased risk as compared to the published gender-specific population generic lifetime risk for that phenotype as described herein) for a specific phenotype, such as a condition, within the Full Genome Analysis Panel (or subset thereof), one or more “reflex” phenotypes, such as conditions, may be tested. For example, if an individual tests positive for Myocardial Infarction, one or more reflex conditions may be tested (as shown in Cardiovascular Panel Beta), such as the effect of consuming a specific type of beverage or food might have on the individual's risk of developing myocardial infarction. Knowledge gained from these tests may help the individual and/or their healthcare provider or third party plan an appropriate diet or, for example, limit his or her alcohol intake, or institute preventive measures and/or interventions to potentially minimize the impact of or avoid the diseases that the individual is found to be at increased risk for.

A positive result for the phenotypes (e.g., diseases, disorders, traits or conditions) of Coronary Artery Disease (CAD) and/or Myocardial Infarction (MI) may reflex to a phenotype, such as a condition, or set of phenotypes, such as conditions, related to an individual's response to a drug or medication to treat or prevent heart disease (including CAD or MI), sensitivity to a drug or medication, metabolism of a drug or medication, response to a particular treatment, or response to a specific medical procedure (e.g., angioplasty, bypass surgery, management with medication). For example, the reflex condition may be adverse reactions to anti-hyperlipidemic medications (such as HMG-CoA reductase inhibitors) or other reflex testing condition listed in FIG. 15. The results may contribute to a pharmacogenomic profile of such individual and may inform treatment approaches including choice of medication and dosage. Thus, an individual with a family or personal medical history of abnormal drug metabolism or adverse reactions to medications or supplements may be interested in being tested with the Full Genome Analysis Panel. An individual with a family or personal medical history of abnormal drug metabolism or adverse reactions to medications or supplements may also be tested with one or more of the following panels (or subset thereof): Full Genome Analysis Panel Alpha (FIG. 15) Full Genome Analysis Panel Beta, or both; the Executive Panel Alpha, Executive Panel Beta, or both; Transplant Panel; Pharmacology & Alternative Medication Panel; or other panels, including panels directed to a specific organ or organ system, as described herein.

A Full Genome Panel Alpha can determine the risk or predisposition of an individual for all the diseases, disorders or traits (also referred herein to as phenotype) listed in FIG. 15, or a subset, such as at least 2, 3, 4, 5, 6, 7, 8, 9, 10, or 11 of the following phenotypes: Heart Disease (including but not limited to Coronary Artery Disease (CAD) and/or Myocardial Infarction and/or Cardiomyopathy); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Thrombophilia and/or Thromboembolic Disease; Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Medication Metabolism and/or Adverse Reactions to Medications (including but not limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Rare Diseases and/or Orphan Diseases and/or Metabolic Diseases and/or Syndromes (such as described above), Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Infectious Disease Susceptibility (Including but Not Limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections); Universal Identifier/Identity Testing; Blood Group; Height and/or Weight (Including but not Limited to Weight, BMI, Obesity, Leanness, Waist Circumference, Adiposity, and Fat Distribution).

A Full Genome Panel Alpha can determine the risk or predisposition may detect the risk or predisposition of a subset of the aforementioned diseases or traits, such as at least 2, 3, 4, 5, or 6 of the following phenotypes: Heart Disease (including but not limited to Coronary Artery Disease (CAD) and/or Myocardial Infarction and/or Cardiomyopathy); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Thrombophilia and/or Thromboembolic Disease; Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Medication Metabolism and/or Adverse Reactions to Medications (including but not limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); or Rare Diseases and/or Orphan Diseases and/or Metabolic Diseases and/or Syndromes. This panel, as with all other panels, can also be run on any genetic material from an embryo or fetus, including but not limited to cells from an amniocentesis or chorionic villus sampling (CVS), or from embryo or fetal genetic material obtained through non-invasive prenatal testing methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, such as the amnion, the amniotic sac, the blood of a pregnant female or via central or peripheral blood draw(s) (such as venipuncture) from the pregnant female.

Thus, the panel may be used to determine an individual's Universal Identifier, which is a unique sequence of multiple genetic variants that are usually not clinically relevant and the unique sequence is exactly specific to only one individual in the entire world. This is similar to a fingerprint but is detectable in any specimen from the individual (e.g. blood, urine, hair, semen, saliva, tissue, etc) that contains genetic material. This can be utilized either to confirm/verify identity (e.g., of an abducted or kidnapped individual or child) or for uses such as to enable confidential or classified or restricted access, to enable corporate and/or personal security, to protect heads-of-state, for government and/or military use or for forensic use. For example, an individual's unique genotype at the genetic variants that constitute the Universal Identifier, can be used to identify or distinguish that individual from all other individuals in the world, with a probability of discrimination that may be greater than 90%, greater that about 95%, 99%, 99.9, or 99.99%. In some cases, the probability of discrimination may be greater than about 99.999, 99.9999, or 99.9999999999% or greater in all populations. The Universal Identifier may be used on a variety of identification items such as on military identification tags (dog tags), on security cards, on documents, on medical records, on tissue specimens, on pathological specimens, on hair, blood, saliva, semen or other bodily fluids, on stored genetic material, identification of individuals in government databases, in personal databases, in corporate databases, in military databases, in criminal databases, or any other use where personal identification with an extremely high degree of certainty and security are needed, wanted or required. This Universal Identifier may represent a minimum set of genetic variants necessary to distinguish one individual from all other individuals in the world out of all populations and therefore genotyping of only these genetic variants may be necessary to confirm, or to rapidly confirm, an individual's identity. The panel may also test for the patients blood group (which may include many different phenotypes along with the ABO blood group system, such as the Duffy Antigen blood group, the Kell blood group, the Colton blood group, the Raph blood group, the P blood group system, and all other known blood groups) based on specific genetic variants in multiple genes and this can be utilized to further confirm identity and also by medical professionals to confirm the patient's exact blood group derived from other laboratory tests, such as another genetic way to further confirm identity or a confirmatory or ancillary indicator of blood group prior to a blood transfusion.

Individuals may also select the Full Genome Panel Beta, which can be used to determine the risk or predisposition for certain phenotypes, such as at least 2, 3, 4, 5, 6, 7, 8, 9, 10, 11, 12, 13, 14, 15, 16, 17, 18, 19, 20, or 21 of the following phenotypes: Myocardial Infarction; Alzheimer's Disease; Malignant Hyperthermia; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions); Lung Cancer; Colorectal Cancer; Stroke (CVA); Cystic Fibrosis; Tay-Sachs Disease; Glucose-6-phosphate Dehydrogenase Deficiency; Hypertrophic Cardiomyopathy; Arrhythmogenic Right Ventricular Cardiomyopathy; Attention Deficit Hyperactivity Disorder; Long QT Syndrome; Wolff-Parkinson-White Syndrome; Thrombophilia and/or Thromboembolic Disease; Melanoma; Macular Degeneration; Sensitivity to UV Light and/or UV-induced Skin Damage and/or Tanning Ability; Androgenic Alopecia; or Traveler's Diarrhea Susceptibility. The Full Genome Panel Beta can also be used to determine the risk or predisposition of a subset of the aforementioned phenotypes, such as at least 2, 3, 4, 5, 6, 7, 8, 19, 10, 11, or 12 of the following phenotypes: Myocardial Infarction; Alzheimer's Disease; Malignant Hyperthermia; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions); Lung Cancer; Colorectal Cancer; Stroke (CVA); Cystic Fibrosis; Tay-Sachs Disease; Glucose-6-phosphate Dehydrogenase Deficiency; Hypertrophic Cardiomyopathy; Arrhythmogenic Right Ventricular Cardiomyopathy. This panel as with all panels, can be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, such as the amnion, the amniotic sac, the blood of a pregnant female or via peripheral or central blood draw(s) from the pregnant female.

Individuals interested in having children may be interested in a carrier screening panel, see, e.g., the Carrier Screening Panel, a rare disease panel, see, e.g. the Rare Disease Screening Panel, and/or a fertility and pregnancy panel, see, e.g., the Female Fertility Panel, Male Fertility and Erectile Function Panel, or the Pregnancy Panel; or if they are thinking of using an egg or sperm donor, or assisted reproductive technologies for a pregnancy, they may be interested in the Reproduction, Egg & Sperm Donor Screening Panel Alpha and/or Beta and/or Assisted Reproductive Technology Panel. A Carrier Screening Panel may be used to test an individual (e.g., a woman, prospective mother, a man, a prospective father, etc.) with a personal medical history of having a disease or history of having a child with a disease. A carrier screening panel may also be used to test an individual with a family history of a disease, such as a debilitating, chronic, or deadly disease (e.g., degenerative neurologic disease or disorder, metabolic disease, cardiac disease, autism, cancer). This panel, as with all other panels, can be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, such as the amnion, the amniotic sac, the blood of a pregnant female or via a peripheral or central blood draw from the pregnant female. Testing either the prospective father or the prospective mother with the Carrier Screening Panel can offer information about potential phenotypes, such as diseases and traits that may affect their future children. Testing both the prospective mother and the prospective father with the Carrier Screening Panel and/or the Rare Disease Screening Panel and combining the results during the analysis may be used to determine the potential phenotypes, such as diseases and traits that may affect their future children, such as by utilizing the OP-CADI. For example, individuals may select the Carrier Screening Panel, which can be used to determine the risk or predisposition of an individual for phenotypes, such as at least 1, 2, 3, 4, 5, 6, 7, 8, 9, 10, or 11 of the following phenotypes: Rare Diseases and/or Orphan Diseases and/or Metabolic Diseases and/or Syndromes; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Mental Retardation and/or Pervasive Developmental Disorder (including but not limited to Autism, Autism Spectrum Disorder, Asperger Syndrome, and/or Rett Syndrome); Structural Heart Defect; Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Hearing Impairment (Including Deafness and/or Hearing Loss); Visual Impairment and/or Visual Acuity (including but not limited to Leber Congenital Amaurosis and/or Macular Degeneration and/or Congenital Blindness and/or Acquired Blindness and/or Myopia and/or Hyperopia and/or Glaucoma and/or Cataracts and/or Visuospatial/Perceptual Abilities and/or Color Perception and/or Color Blindness and/or Night Blindness); Skeletal Abnormalities and/or Appendage Abnormalities; Immune Status and/or Immunodeficiency; or Myopathies and/or Muscular Atrophy and/or Muscular Dystrophy and/or Neuropathies and/or Charcot-Marie-Tooth Disease.

The Carrier Screening Panel can also be used to determine the risk or predisposition of a subset of the aforementioned phenotypes, such as at least 1, 2, 3, or 4 of the following phenotypes: Rare Diseases and/or Orphan Diseases and/or Metabolic Diseases and/or Syndromes; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); or Mental Retardation and/or Pervasive Developmental Disorder (including but not limited to Autism, Autism Spectrum Disorder, Asperger Syndrome, and/or Rett Syndrome).

If an individual tests positive for an initial phenotype, such as a condition, phenotypes, such as conditions, related to the initial phenotype, such as a condition, may be reflexively tested with a carrier screening panel. The phenotypes, such as conditions, reflexively tested may provide information to the prospective parent, such as further information about the nature of the initial phenotype, such as a condition. For example, if an individual tests positive for an initial condition that is a disease (e.g., Parkinson's Disease), then the phenotype, such as a condition, that can be reflexively tested may be the age of onset of the disease (e.g., age of onset of Parkinson's Disease), or the risk of certain symptoms associated with such disease (e.g., the risk of incurring certain symptoms associated with Parkinson's Disease, such as the risk of developing motor fluctuations or the risk of sudden sleep onset such as sleep attacks, or both). Prospective parents may be interested in all of the conditions of one of the panels, or in more than one of the panels. Alternatively, they may be interested in a subset of conditions of a single panel or of two or more panels. In some cases, a carrier screening panel may be used to test an individual for a set of two or more risks (the term ‘risk’ may refer to either or both: risk of multifactorial phenotype(s) or carrier status of monogenic or polygenic phenotype(s), which includes whether the person is a carrier, a non-carrier, or affected or likely affected by the phenotype(s)), for example, such set may include one of the following sets of risks or predispositions: risk for pervasive developmental disorder (e.g., autism, autism spectrum disorder, Asperger Syndrome, Rett Syndrome, etc.) and risk of neurodegenerative disease or disorder (e.g., Alzheimer's Disease, Parkinson's Disease, etc.); risk for pervasive developmental disorder and risk of specific conditions correlated with sudden death; risk of pervasive developmental disorder and risk of metabolic disease; risk of cardiac arrhythmia and risk of mental retardation; risk of structural heart defect and risk of breast cancer; or risk of neurodegenerative disease or disorder and risk of mental retardation.

The Female Fertility Panel, Male Fertility and Erectile Function Panel, or the Pregnancy Panel; the Assisted Reproductive Technology Panel, or the Miscarriage, Spontaneous Abortion, or Difficulty Conceiving Panel may be useful to individuals with a family or personal medical history of irregular or absent menstrual cycles, abnormalities with ovulation, erectile dysfunction, difficulties conceiving (for example, difficulties due to structural abnormalities with reproductive organs or abnormal egg or sperm morphology, motility, quantity or quality), infertility, or complications associated with pregnancy such as preterm birth, miscarriage, preeclampsia, eclampsia, or hypertension during pregnancy or a history of wound dehiscence. The entire panel may be selected, or a subset.

For example, individuals may select the Female Fertility Panel, which can be used to determine the risk or predisposition of an individual for phenotypes, such as at least 1, 2, 3, 4, 5, 6, or 7 of the following phenotypes: Female Fertility/Infertility and/or Spontaneous Abortion and/or Miscarriages; Ovulatory Defects and/or Premature Ovarian Failure and/or Ovarian Dysgenesis; Thrombophilia and/or Thromboembolic Disease; Fetal Viability; Bleeding Diathesis and/or Coagulation Disorders and/or Hemophilia; Primary and/or Secondary Sex Characteristics and/or Sex Reversal and/or Hypogonadism; or Hypogonadism. A Female Fertility Panel can determine the risk or predisposition of a subset of the aforementioned phenotypes, such as at least 1, 2 or 3 of the following phenotypes: Female Fertility/Infertility and/or Spontaneous Abortion and/or Miscarriages Ovulatory Defects and/or Premature Ovarian Failure and/or Ovarian Dysgenesis; or Thrombophilia and/or Thromboembolic Disease. Individuals with or without a current or prior diagnosis of infertility or difficulty conceiving may also be interested in other genetic links to phenotypes, and their risk or predisposition to those phenotypes, that are related to infertility or difficulty conceiving and thus may also be interested in the Female Fertility Panel, for example.

In other embodiments, individuals may select the Male Fertility & Erectile Function Panel, which can be used to determine the risk or predisposition of an individual for phenotypes, such as at least 1, 2, 3, 4, 5 or 6 of the following phenotypes: Male Fertility/Infertility (including but not limited to Abnormal Sperm Count and/or Abnormal Sperm Motility and/or Abnormal Sperm Morphology); Erectile Dysfunction Medication Treatment Effectiveness and/or Sensitivity; Peripheral Arterial Disease; Fetal Viability; Primary and/or Secondary Sex Characteristics and/or Sex Reversal and/or Hypogonadism; or Hypogonadism. A Male Fertility & Erectile Function Panel can determine the risk or predisposition of a subset of the aforementioned phenotypes, such as at least 1 or 2 of the following phenotypes: Male Fertility/Infertility (including but not limited to Abnormal Sperm Count and/or Abnormal Sperm Motility and/or Abnormal Sperm Morphology); or Erectile Dysfunction Medication Treatment Effectiveness and/or Sensitivity. Individuals with or without a current or prior diagnosis of erectile dysfunction, infertility or difficulty conceiving may also be interested in other genetic links to phenotypes, and their carrier status, risk or predisposition to those phenotypes, that are related to erectile dysfunction, infertility or difficulty conceiving and thus may also be interested in the Male Fertility & Erectile Function Panel, for example.

Individuals may also select the Pregnancy Panel, which can be used to determine the risk or predisposition of an individual for phenotypes, such as at least 1, 2, 3, 4, 5, or 6 of the following phenotypes: Risk of Preterm Birth; Preeclampsia and/or Eclampsia and/or Hypertension during Pregnancy; Wound Dehiscence; Bleeding Diathesis and/or Coagulation Disorders and/or Hemophilia; Thrombophilia and/or Thromboembolic Disease; Thromboembolism during Pregnancy; or Fetal Viability. A Pregnancy Panel can determine the risk or predisposition of a subset of the aforementioned phenotypes, such as at least 1, 2 or 3 of the following phenotypes: Risk of Preterm Birth; Preeclampsia and/or Eclampsia and/or Hypertension during Pregnancy; or Wound Dehiscence. Individuals with or without a current or prior diagnosis of pregnancy or who are trying to get pregnant may also be interested in other genetic links to phenotypes, and their risk or predisposition to those phenotypes, that are related to pregnancy and thus may also be interested in the Pregnancy Panel, for example.

Individuals can also select the Assisted Reproductive Technology Panel, which can be used to determine the risk or predisposition of an individual for phenotypes, such as at least 1, 2, 3, 4, 5, 6, or 7 of the following phenotypes: Dosage of Follicle-Stimulating Hormone (FSH) Needed to Obtain Good-quality Embryo for In-Vitro Fertilization (IVF); Number of Retrieved Oocytes after Ovarian Stimulation and/or Effectiveness of Controlled Ovarian Hyperstimulation; Risk of Twinning; Thrombophilia and/or Thromboembolic Disease; Ovarian Hyperstimulation during In-Vitro Fertilization (IVF); Ovarian Response to Follicle-Stimulating Hormone (FSH) Stimulation; or Fetal Viability. An Assisted Reproductive Technology Panel can determine the risk or predisposition of a subset of the aforementioned phenotypes, such as at least 1, 2 or 3 of the following phenotypes: Dosage of Follicle-Stimulating Hormone (FSH) Needed to Obtain Good-quality Embryo for In-Vitro Fertilization (IVF); Number of Retrieved Oocytes after Ovarian Stimulation and/or Effectiveness of Controlled Ovarian Hyperstimulation; or Risk of Twinning. Individuals with or without a current or prior diagnosis of a miscarriage, spontaneous abortion, or who are having difficulty conceiving may also be interested in other genetic links to phenotypes, and their risk or predisposition to those phenotypes, that are related to miscarriage, spontaneous abortion, or difficult conceiving and thus may also be interested in the Assisted Reproductive Technology Panel, for example.

Individuals may select the Miscarriage, Spontaneous Abortion, or Difficulty Conceiving Panel, which can be used to determine the risk or predisposition of an individual for phenotypes, such as at least 1, 2, 3, 4, 5, or 6 of the following phenotypes: Female Fertility/Infertility and/or Spontaneous Abortion and/or Miscarriages and/or Reproduction System Abnormalities; Fetal Viability; Ovarian Abnormalities and/or Ovulatory Abnormalities; Thrombophilia and/or Thromboembolic Disease; Bleeding Diathesis and/or Coagulation Disorders and/or Hemophilia; or Male Fertility/Infertility (including but not limited to Abnormal Sperm Count and/or Abnormal Sperm Motility and/or Abnormal Sperm Morphology). Individuals with or without a current or prior diagnosis of a miscarriage, spontaneous abortion, or who are having difficulty conceiving may also be interested in other genetic links to phenotypes, and their risk or predisposition to those phenotypes, that are related to miscarriage, spontaneous abortion, or difficult conceiving and thus may also be interested in the Miscarriage, Spontaneous Abortion, or Difficult Conceiving Panel, for example.

A Miscarriage, Spontaneous Abortion, or Difficulty Conceiving Panel can determine the risk or predisposition of a subset of the aforementioned phenotypes, such as at least 1, 2, 3, 4, or 5 of the following phenotypes: Female Fertility/Infertility and/or Spontaneous Abortion and/or Miscarriages and/or Reproduction System Abnormalities; Fetal Viability; Ovarian Abnormalities and/or Ovulatory Abnormalities; Thrombophilia and/or Thromboembolic Disease; or Bleeding Diathesis and/or Coagulation Disorders and/or Hemophilia. Results can lead to a reflex testing for the effectiveness of and/or sensitivity to medications used to treat erectile dysfunction.

Individual(s) with difficulties in conceiving or who are biologically unable to have a child (such as women who have had their ovaries removed to treat cancer or men with azoospermia or same-sex couples) may be interested in using eggs or sperm from donors, and may be concerned about potential diseases and/or traits that may affect children from egg and/or sperm donor. They may opt for the use of Reproduction, Egg & Sperm Donor Screening Panel Alpha and/or Beta for testing either the donors themselves (e.g., the female egg donor and/or the male sperm donor) or by selecting a haploid genome (e.g., the actual sperm or egg cells). A cell of male origin, such as sperm, or a cell of female origin, such as an oocyte, may be applied to the panel and the genetic polymorphism profile of the cell determined. The results may be used to select the sperm and/or egg to be used to produce a diploid embryo, such as in in vitro fertilization. Other factors, such as the gender, ethnicity, age, weight, body mass index, lifestyle habits (smoking, drinking, etc.), biomarkers or blood levels or serum concentrations of specific substances, such as serum 25-hydroxyvitamin D, medications and alternative therapies such as herbology, family history of disease and/or personal history of disease (both past and current) of the egg/sperm donor may also be incorporated into the results. The Reproduction, Egg & Sperm Donor Screening Panel may be used to determine the risk or predisposition of a donor for a particular disease or condition when there is limited or no information about the donor, or when the donor's family or medical history is limited or unavailable. The entire panel may be selected, or a subset.

For example, individuals may select the Reproduction, Egg & Sperm Donor Screening Panel Alpha, which can be used to determine the risk or predisposition of an individual for phenotypes, such as at least 1, 2, 3, 4, 5, 6, 7, 8, 9, 10, 11, 12, 13, 14, 15, 16, or 17 of the following phenotypes: Height and/or Weight (Including but not Limited to Weight, BMI, Obesity, Leanness, Waist Circumference, Adiposity, and Fat Distribution); Longevity and/or Lifespan; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Primary and/or Secondary Sex Characteristics and/or Sex Reversal and/or Hypogonadism; Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Physical Traits (Including but not Limited to Ethnicity and/or Eye Color and/or Skin Color and/or Skin Pigmentation and/or UV Sensitivity and/or Tanning Response to Sunlight and/or Freckling and/or Size of External Genitalia and/or Mole Count and/or Hair Color and/or Hair Thickness); Mental Retardation and/or Pervasive Developmental Disorder (including but not limited to Autism, Autism Spectrum Disorder, Asperger Syndrome, and/or Rett Syndrome); Rare Diseases and/or Orphan Diseases and/or Metabolic Diseases and/or Syndromes; Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders); Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Skeletal Abnormalities and/or Appendage Abnormalities; Hearing Impairment (Including Deafness and/or Hearing Loss); Visual Impairment and/or Visual Acuity (including but not limited to Leber Congenital Amaurosis and/or Macular Degeneration and/or Congenital Blindness and/or Acquired Blindness and/or Myopia and/or Hyperopia and/or Glaucoma and/or Cataracts and/or Visuospatial/Perceptual Abilities and/or Color Perception and/or Color Blindness and/or Night Blindness); or Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections). The Reproduction, Egg & Sperm Donor Screening Panel Alpha can determine the risk or predisposition of a subset of the aforementioned phenotypes, such as at least 1, 2, 3, 4, 5, 6, 7, 8, or 9 of the following phenotypes: Height and/or Weight (Including but not Limited to Weight, BMI, Obesity, Leanness, Waist Circumference, Adiposity, and Fat Distribution); Longevity and/or Lifespan; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Primary and/or Secondary Sex Characteristics and/or Sex Reversal and/or Hypogonadism; Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Physical Traits (Including but not Limited to Ethnicity and/or Eye Color and/or Skin Color and/or Skin Pigmentation and/or UV Sensitivity and/or Tanning Response to Sunlight and/or Freckling and/or Size of External Genitalia and/or Mole Count and/or Hair Color and/or Hair Thickness); Mental Retardation and/or Pervasive Developmental Disorder (including but not limited to Autism, Autism Spectrum Disorder, Asperger Syndrome, and/or Rett Syndrome); or Rare Diseases and/or Orphan Diseases and/or Metabolic Diseases and/or Syndromes.

Each of these phenotypes is associated with genetic variants that provide some level of associated risk for having the phenotype. In some instances the risk is related to the degree of a phenotype. For example, in some cases the BMI linkage may be either an association with a relatively low or high BMI. In some cases, the risk or predisposition is provided for the likelihood of developing an extreme manifestation of one or more phenotypes, such as for example extreme high or low BMI, extreme height (tall or short), or extreme intelligence (mentally retarded or gifted). The term extreme may refer to phenotypes that are beyond the normal range. For example, in terms of standard deviation, extreme may refer to phenotypes such, for example, as height, weight, BMI, longevity, sports aptitude or intelligence that are 98% above the mean population or below 2% of the mean. For example, extreme longevity may represent semi-supercentenarians (age above 105). As another example, for pre-term infants, extremely pre-term may mean gestation less than about 28 weeks. In another example, the genetic variants associated with an optimal exercise regimen may reflect a predisposition to ability to build muscle mass; a high or low degree of hand eye coordination; a susceptibility to bone, muscle, joint, or tendon/ligament injuries; or endurance capabilities which can be leveraged into a designed exercise program suited to the individual. Alternatively, the genetic variants associated with an optimal exercise regimen may provide an indicator of long-term prognosis and/or dementia or Alzheimer's Disease susceptibility following head and/or brain injury. A parent, guardian, insurance company, government agency, coach, or other athletic official may use this information to determine whether or not the child should participate in contact sports or physical activity that may result in a head injury, such as but not limited to ice hockey, field hockey, soccer, football, lacrosse, wrestling, bike riding, pole vaulting, roller-blading, skate boarding, surfing or boxing as they may increase the risk of head trauma and brain injury.

For height and weight, a large set of genetic variants add a certain amount of height (or don't add height, depending on the genotype) and many are additive, so that one may add 0.7 cm, two may add 1.4 cm, etc, so expected height and weight/bmi values provided by the methods of the present invention may be in-relation to other possible genotypes (such as “taller by 1.4 cm” or “shorter by 2.8 cm”. There are also other genetic variant groups that when they occur together, the individuals may be approximately 3.5 cm shorter than average while other groups may be delineated 3.5 cm taller than average height. So, the methods of the present invention provide for predicting whether an individual is likely to be “tall stature”, “normal stature” or “short stature”, as well as their adult height ranges. The predicted phenotypes provided herein for adult height may include phenotypes such as tall (such as ≧5′ 10 of men or 25′8 for women) or Short (such as ≦5′5 for men or ≦5′2 for women) or it can refer to specific numerical value range, such as a number range of 2-4 inches between 4′5 and 7′ 1, such as 5′7-5′9 or 5′2-5′5. Similarly, genetic variants associated with phenotypes provided herein for adult body mass index (BMI) may genetic variants that predict a BMI category, such as, for example, Severely underweight (<16.5), Underweight (16.5-18.5), Normal (18.5-25), Overweight (25-30), Obese Class I (30-35), Obese Class II (35-40), and Obese Class III (>40). Increased BMI refers to a BMI above normal (such as >25) and a Lower BMI refers to a BMI between 17-24. Similarly, genetic variants associated with phenotypes provided herein for adult weight may predict a weight range, such as 120-130 lb, or a weight category, such as, for example, for a 5 foot 11 inches tall person: Severely underweight (<118 lb), Underweight (118-130 lb), Normal (130-180), Overweight (180-210 lb), Obese Class I (210-250 lb), Obese Class II (250-290 lb), and Obese Class III (>290 lb). Increased weight refers to a weight above normal (with normal weight being defined by gender and height) and decreased weight or lower weight refers to weight that is slightly underweight to normal (with normal weight being defined by gender and height). Similarly genetic variants associated with phenotypes provided herein for childhood weight, newborn weight, and childhood length (height) may include genetic variants that predict a range, such as a weight range, such as 10-15 lb, or length range, such as 2 ft-2 ft 5 inches, or genetic variants that predict a category, such as a weight category or a length category, such as, for example, percentile categories as per United States Centers of Disease Control's Clinical Growth Charts for Boys and Girls (http://www.cdc.gov/nchs/about/major/nhanes/growthcharts/clinical_charts.htm#Clin%201). Weight may also be predicted to be underweight if the child's BMI is less than the 35th percentile, normal if the child's BMI is between the 35-85 percentile, overweight if the child's BMI is between the 85-95 percentile or obese if the child's BMI is greater than the 95th percentile.

Other phenotypes that can be predicted by the methods provided herein include diurnal preference which includes whether an individual may be more alert or prefer to be more active or awake during the morning, afternoon, late afternoon, or evening: Also provided herein are methods for predicting phenotypes related to stress, stress levels, response to stress, and/or anxiety. Stress levels may be measured for example by endogenous opioid neurotransmission after a stressful or painful stimuli, such as by studying the stress-induced m-opioid system activation in several brain regions including prefrontal cortex, posterior insula, medial and lateral thalamus, ventral basal ganglia (ventral caudate, ventral putamen and nucleus accumbens) and amygdala. Stress and response to stress can also be evaluated through self-rated pain and affective response such as subjective pain (McGill Pain Questionnaire sensory subscale) and emotional experience (Positive and Negative Affectivity Scale). Anxiety may be measured with the Tridimensional Personality Questionnaire (TPQ) Harm Avoidance subscales Fear of Uncertainty, Anticipatory Worry, Shyness with Strangers, and Fatigability and Asthenia. Clinical anxiety disorders may be diagnosed with the Structured Clinical Interview for the Diagnostic and Statistical Manual of Mental Disorders, 3rd edition revised. Also provided herein are methods related to predicting intelligence. Intelligence may refer to cognitive ability and/or intelligence quotient (IQ), and may refer to either very low intelligence or high intelligence or it may refer to specific IT ranges and/or specific intelligence categories, such as an IQ score of 1-24 (Profound Mental Disability), 25-39 (Severe Mental Disability), 40-54 (Moderate Mental Disability), 55-69 (Mild Mental Disability), 70-84 (Borderline Mental Disability), 85-114 (Average Intelligence), 115-129 (Bright), 130-144 (Moderately Gifted), 145-159 (Highly Gifted), 160-175 (Exceptionally Gifted), and Over 175 (Profoundly Gifted). Alternatively, genetic variants can provide intelligence phenotypes that are additive or subtractive such as for example Increased Risk for Increased Cognitive Ability, Such as Having a Higher IQ (such as ˜7 IQ points Higher) in Adulthood (e.g. 18 Years Old and Older); Increased Risk for Decreased Cognitive Ability, Such as Having a Lower IQ (e.g. ˜7 IQ points Lower) in Adulthood (e.g. 18 Years Old and Older); Increased Risk for Increased Cognitive Ability, Such as Having a Higher IQ (e.g. ˜6 IQ points Higher) in Childhood (eg. Younger than 18 Years Old); Increased Risk for Decreased Cognitive Ability, Such as Having a Lower IQ (e.g. ˜6 IQ points Lower) in Childhood (e.g. Younger than 18 Years Old). Exemplary genetic variants related to intelligence or IQ include but are not limited to variants in or in linkage disequilibrium with CHRM2, and SNAP-25 (see e.g. Dick, D., F. Aliev, et al. (2007). “Association of CHRM2 with IQ: Converging Evidence for a Gene Influencing Intelligence.” Behavior Genetics 37(2): 265-272; M. F. Gosso, M. v. B. E. J. C. d. G. J. C. P. P. H. D. I. B. D. P. (2006). “Association between the CHRM2 gene and intelligence in a sample of 304 Dutch families.” Genes, Brain and Behavior 5(8): 577-584; and M. F. Gosso, E. J. C. d. G. T. J. C. P. D. I. B. P. H. D. P. (2008). “Common variants underlying cognitive ability: further evidence for association between the SNAP-25 gene and cognition using a family-based study in two independent Dutch cohorts.” Genes, Brain and Behavior 7(3): 355-364).

In some embodiments, individuals may select the Reproduction, Egg & Sperm Donor Screening Panel Beta, which can be used to determine the risk or predisposition of an individual for phenotypes, such as at least 1, 2, 3, 4, 5, 6, 7, 8, 9, 10, 11, or 12 of the following phenotypes: Longevity and/or Lifespan; Dilated Cardiomyopathy; Intelligence (IQ); Athletic Ability; Autism; Breast Cancer; Sudden Infant Death Syndrome; Mental Retardation; Parkinson Disease; Breast Cancer; Cystic Fibrosis; or Arrhythmogenic Right Ventricular Cardiomyopathy. A Reproduction, Egg & Sperm Donor Screening Panel Beta can determine the risk or predisposition of a subset of the aforementioned phenotypes, such as at least 1, 2, 3, 4, or 5 of the following phenotypes: Longevity and/or Lifespan; Dilated Cardiomyopathy; Intelligence (IQ); Athletic Ability; or Autism. Individuals may choose to select both Alpha and Beta panels.

Pregnant women or women considering pregnancy may also be interested in the Embryo and Fetus Panel Alpha and/or Beta. For example, the Embryo and Fetus Panel may be used following, or in addition to, or with, an abnormal fetal ultrasound or an abnormal maternal-fetal blood test result or after a conception occurs. Women with a history of stillbirth, miscarriage, or having children with a disease may also be tested with the Embryo and Fetus Panel. The panel can be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, such as the amnion, the amniotic sac, the blood of a pregnant female or via a peripheral blood drawn from the pregnant female. The entire panel may be selected, or a subset. For example, pregnant women, women considering conceiving children, their partners, their family, medical centers, and/or health care providers may select the Embryo and Fetus Panel Alpha, which can be used to determine the carrier status and/or risk or predisposition of an individual for phenotypes, such as at least 1, 2, 3, 4, 5, 6, 7, 8, 9, 10, 11, or 12 of the following phenotypes: Gender; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Effect of Breast Feeding upon Intelligence (IQ); Primary and/or Secondary Sex Characteristics and/or Sex Reversal; Rare Diseases and/or Orphan Diseases and/or Metabolic Diseases and/or Syndromes; Paternity; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Mental Retardation and/or Pervasive Developmental Disorder (including but not limited to Autism, Autism Spectrum Disorder, Asperger Syndrome, and/or Rett Syndrome); Universal Identifier and Blood Group; Physical Traits (Including but not Limited to Ethnicity and/or Eye Color and/or Skin Color and/or Skin Pigmentation and/or UV Sensitivity and/or Tanning Response to Sunlight and/or Freckling and/or Size of External Genitalia and/or Mole Count and/or Hair Color and/or Hair Thickness); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); or Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury). The Embryo and Fetus Panel Alpha can also be used to determine the risk or predisposition of a subset of the aforementioned phenotypes, such as at least 1, 2, 3, 4, or 5 of the following phenotypes: Gender; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Effect of Breast Feeding upon Intelligence (IQ); or Primary and/or Secondary Sex Characteristics and/or Sex Reversal; or Rare Diseases and/or Orphan Diseases and/or Metabolic Diseases and/or Syndromes.

Individuals can also select the Embryo and Fetus Panel Beta, which can be used to determine the carrier status and/or risk or predisposition of an individual for phenotypes, such as at least 1, 2, 3, 4, 5, 6, 7, 8, 9, 10, 11, 12, 13, 14, 15, 16, 17, 18, 19, 20, or 21 of the following phenotypes: Autism; Mental Retardation; Sudden Infant Death Syndrome; Intelligence (IQ); Effect of Breast Feeding upon Intelligence (IQ); Wolff-Parkinson-White Syndrome; Hypertrophic Cardiomyopathy; or Arrhythmogenic Right Ventricular Cardiomyopathy. An Embryo and Fetus Panel Beta can determine the risk or predisposition of a subset of the aforementioned phenotypes, such as at least 1, 2 or 3 of the following phenotypes: Autism; Mental Retardation; or Sudden Infant Death Syndrome.

Parents or guardians may be interested in determining the carrier status and/or degree of risk of phenotypes, such as conditions (e.g., diseases, disorders or traits) of their children, such as a child under approximately 2, 3, 5, 4, 5, 6, 7, 8, 9, 10, 11, 12, 13, 14, 15, 16, 17, or 18 years of age, and thus may submit their child's sample or specimen for testing. Testing may be with the Newborn Panel Alpha and/or Beta, Pediatric Panel Alpha and/or Beta, the Preterm Infant Panel, and/or the Pediatric Psychiatry Panel. The individual may be tested with the entire panel or subset thereof. As with all of the panels, these panels can be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, such as the amnion, the amniotic sac, the blood of a pregnant female or via peripheral or central blood draw(s) from the pregnant female.

For example, parents or guardians may be interested in the Pediatric Panel for their newborn or child between the ages of 0-19. This panel may be useful for parents or schools or athletic organizations (such as high school or city or state or national or professional sports teams) if, for instance, the child may participate in contact sports or activities. The Pediatric Panel provides an indicator of long-term prognosis and/or dementia or Alzheimer's Disease susceptibility following head and/or brain injury. A parent, guardian, insurance company, coach, or other athletic official may use this information to determine whether or not the child should participate in contact sports or physical activity that may result in a head injury, such as ice hockey, field hockey, soccer, football, lacrosse, wrestling, bike riding, pole vaulting, roller-blading, skate boarding, surfing or boxing as they may increase the risk of head trauma and brain injury. Alternatively, a mother or nurse may be interested in the Pediatrics Panel if they are deciding whether or not to breastfeed, as the Pediatrics Panel can supply them with information about whether or not breast feeding will increase their child's intelligence quotient (IQ).

A parent may select the Pediatric Panel Alpha, which can be used to determine the risk or predisposition of an individual for phenotypes, such as at least 1, 2, 3, 4, 5, 6, 7, 8, 9, 10, 11, 12, 13, 14, or 15 of the following phenotypes: Universal Identifier and Blood Group; Effect of Breast Feeding upon Intelligence (IQ); Learning Issues (including but not limited to Attention Deficit Hyperactivity Disorder and/or Dyslexia and/or Reading Performance Ability); Pervasive Developmental Disorder (including but not limited to Autism, Autism Spectrum Disorder, Asperger Syndrome, and/or Rett Syndrome); Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury); Height and/or Weight (Including but not Limited to Weight, BMI, Obesity, Leanness, Waist Circumference, Adiposity, and Fat Distribution); Asthma; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Lactose Tolerance or Intolerance; Noise-induced Hearing Impairment and/or Hearing Loss; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections); or Taste Perception and/or Specific Food Preference (including but not limited to Aversion to Eating Vegetables and/or Higher or Lower Consumption of Specific Foods and/or Beverages). A Pediatric Panel Alpha can determine the risk or predisposition of a subset of the aforementioned phenotypes, such as at least 1, 2, 3, 4, 5, or 6 of the following phenotypes: Universal Identifier and Blood Group; Effect of Breast Feeding upon Intelligence (IQ); Learning Issues (including but not limited to Attention Deficit Hyperactivity Disorder and/or Dyslexia and/or Reading Performance Ability); Pervasive Developmental Disorder (including but not limited to Autism, Autism Spectrum Disorder, Asperger Syndrome, and/or Rett Syndrome); Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury); or Height and/or Weight (Including but not Limited to Weight, BMI, Obesity, Leanness, Waist Circumference, Adiposity, and Fat Distribution).

Parents may also select the Pediatric Panel Beta alone, or in combination with the Alpha panel, or other panels disclosed herein. The Pediatric Panel Beta can be used to determine the risk or predisposition of an individual for phenotypes, such as at least 1, 2, 3, 4, 5, 6, 7, 8, 9, 10, 11, or 12 of the following phenotypes: Arrhythmogenic Right Ventricular Cardiomyopathy; Attention Deficit Hyperactivity Disorder; Dyslexia; Intelligence (IQ); Athletic Ability; Prognosis following Head Injury and/or Brain Injury (including but not limited to Cognitive Performance and/or Dementia and/or Alzheimer's Disease Susceptibility); Allergies and/or Atopy (including but not limited to Food Allergies and/or Environmental Allergies and/or Contact Allergies and/or Rashes and/or Eczema); Otitis; Noise-induced Hearing Impairment and/or Hearing Loss; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Long QT Syndrome; or Hypertrophic Cardiomyopathy. A Pediatric Panel Beta can determine the risk or predisposition of a subset of the aforementioned phenotypes, such as at least 1 or 2 or 3 of the following phenotypes: Arrhythmogenic Right Ventricular Cardiomyopathy; Attention Deficit Hyperactivity Disorder; or Dyslexia.

Parents, or relatives, may also choose the Newborn Panel Alpha and/or Beta or the Preterm Infant Panel. The entire Preterm Infant Panel or Newborn Panel(s), or subsets thereof, can be tested. For example, an individual may select the Preterm Infant Panel, which can be used to determine the risk or predisposition of an individual for phenotypes, such as at least 1, 2, 3, 4, or 5 of the following phenotypes: Viability and/or Health Status of Preterm Infants; Pulmonary Function and/or Disease (including but not limited to Respiratory Distress Syndrome in Preterm Infants); Preterm Infant's Susceptibility to Sepsis and/or Severe Sepsis and/or Septic Shock; Risk of Preterm Birth; or Thrombophilia and/or Thromboembolic Disease. A Preterm Infant Panel can determine the risk or predisposition of a subset of the aforementioned phenotypes, such as at least 1, 2, or 3 of the following phenotypes: Viability and/or Health Status of Preterm Infants; Pulmonary Function and/or Disease (including but not limited to Respiratory Distress Syndrome in Preterm Infants); or Preterm Infant's Susceptibility to Sepsis and/or Severe Sepsis and/or Septic Shock.

Individuals can also select the Newborn Panel Alpha, which can be used to determine the risk or predisposition of an individual for phenotypes, such as at least 1, 2, 3, 4, or 5 of the following phenotypes: Universal Identifier and Blood Group; Drug Metabolism and/or Choice and/or Sensitivity and/or Adverse Reactions and/or Dosing (Including Pharmacogenomic Analysis for all Pharmaceuticals); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Thrombophilia and/or Thromboembolic Disease; or Pyloric Stenosis. A Newborn Panel Alpha can determine the risk or predisposition of a subset of the aforementioned phenotypes, such as at least 1, 2, or 3 of the following phenotypes: Universal Identifier and Blood Group; Drug Metabolism and/or Choice and/or Sensitivity and/or Adverse Reactions and/or Dosing (Including Pharmacogenomic Analysis for all Pharmaceuticals); or Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome).

Individuals may select the Newborn Panel Beta, which can be used to determine the risk or predisposition of an individual for phenotypes, such as at least 1, 2, 3, 4, or 5 of the following phenotypes: Sudden Infant Death Syndrome; Arrhythmogenic Right Ventricular Cardiomyopathy; Lactose Tolerance or Intolerance; Thrombophilia and/or Thromboembolic Disease; or Universal Identifier. A Newborn Panel Beta can determine the risk or predisposition of a subset of the aforementioned phenotypes, such as at least 1, 2, or 3 of the following phenotypes: Sudden Infant Death Syndrome; Arrhythmogenic Right Ventricular Cardiomyopathy (also known as Arrhythmogenic Right Ventricular Dysplasia or Naxos Disease or Naxos Syndrome); or Lactose Tolerance or Intolerance.

Parents, or other individuals, such as school or educational officials, employers, and the like, may also be interested in the Behavior & Aptitude Assessment Panel, which can be used to determine the risk or predisposition of an individual for phenotypes, such as at least 1, 2, 3, 4, 5, 6, 7, 8, or 9 of the following phenotypes: Extroversion or Introversion Personality; Violent Behavior; Intelligence (IQ); Athletic Ability; Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders); Mental Vulnerability to Social Stressors and Chronic Disease; Stressful Life Events causing Depressive Symptoms and/or Diagnosable Depression and/or Suicidality and/or Anxiety (including but not limited to Mental Vulnerability to Stress and/or Disease); Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); or Personality Traits (Including but not Limited to Handling of Stress, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance). A Behavior & Aptitude Assessment Panel can determine the risk or predisposition of a subset of the aforementioned phenotypes, such as at least 1, 2, 3 of the following phenotypes: Extroversion or Introversion Personality; Violent Behavior; Intelligence (IQ); or Athletic Ability.

For the elderly, geriatric and aging panes may be selected, see, e.g., the Golden Panel Alpha and/or Beta [Geriatric and Aging Panel Alpha or Beta]. A geriatric and aging panel may be used by the individual themselves, a nursing home, hospice, hospital, or other such facility to test an elderly individual for his or her risk or predisposition for a specific phenotype(s), such as condition(s). Similarly, a medical professional, physician, gerontologist, geriatrician, caretaker, nurse, guardian, private, public or governmental health, disability, life, or any of type of insurance program(s) or organizations (such as government health insurance or government health services and programs, Medicare, Medicaid, or Medi-cal) or other third party may be interested in testing an individual using a geriatric and aging panel. The Golden Panel, or subset thereof, may be used to test an aging individual suffering from a chronic disease such as osteoarthritis or abnormal lipid level or suffering from symptoms such as pain or fatigue (see FIG. 21B).

For example, the Golden Panel Alpha [Geriatric and Aging Panel Alpha] can be used to determine the risk or predisposition of an individual for phenotypes, such as at least 1, 2, 3, 4, 5, 6, 7, 8, 9, 10, 11, 12, 13, or 14 of the following phenotypes: Hearing Acuity (Including but not Limited to Age-related Hearing Impairment and/or Noise-induced Hearing Impairment); Visual Impairment and/or Visual Acuity (including but not limited to Leber Congenital Amaurosis and/or Macular Degeneration and/or Congenital Blindness and/or Acquired Blindness and/or Myopia and/or Hyperopia and/or Glaucoma and/or Cataracts and/or Visuospatial/Perceptual Abilities and/or Color Perception and/or Color Blindness and/or Night Blindness); Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Stroke (CVA); Heart Disease (including but not limited to Coronary Artery Disease (CAD) and/or Myocardial Infarction); Alzheimer's Disease; Osteoporosis and/or Osteoporotic Fracture; Osteoarthritis; Skin Cancer (Including Melanoma or Non-Melanoma Skin Cancer) and/or Sensitivity to UV Light; Colorectal Cancer; Breast Cancer and/or Ovarian Cancer; Prostate Cancer; Thrombophilia and/or Thromboembolic Disease; or Lumber Disc Disease. A Golden Panel Alpha [Geriatric and Aging Panel Alpha] can determine the risk or predisposition of a subset of the aforementioned phenotypes, such as at least 1, 2, 3, 4, 5, 6, 7, 8, or 9 of the following phenotypes: Hearing Acuity (Including but not Limited to Age-related Hearing Impairment and/or Noise-induced Hearing Impairment); Visual Impairment and/or Visual Acuity (including but not limited to Leber Congenital Amaurosis and/or Macular Degeneration and/or Congenital Blindness and/or Acquired Blindness and/or Myopia and/or Hyperopia and/or Glaucoma and/or Cataracts and/or Visuospatial/Perceptual Abilities and/or Color Perception and/or Color Blindness and/or Night Blindness); Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Stroke (CVA); Heart Disease (including but not limited to Coronary Artery Disease (CAD) and/or Myocardial Infarction); Alzheimer's Disease; Osteoporosis and/or Osteoporotic Fracture; Osteoarthritis; or Skin Cancer (Including Melanoma or Non-Melanoma Skin Cancer) and/or Sensitivity to UV Light.

Individuals may select the Golden Panel Beta [Geriatric and Aging Panel Beta], which can be used to determine the risk or predisposition of an individual for phenotypes, such as at least 1, 2, 3, 4, 5, 6, 7, or 8 of the following phenotypes: Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Lumber Disc Disease; Osteoarthritis; Myocardial Infarction; Osteoporosis and/or Osteoporotic Fracture; Stroke (CVA); Alzheimer's Disease; or Coronary Artery Disease (CAD). A Golden Panel Beta [Geriatric and Aging Panel Beta] can determine the risk or predisposition of a subset of the aforementioned phenotypes, such as at least 1, 2, 3, or 4 of the following phenotypes: Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Lumber Disc Disease; Osteoarthritis; or Myocardial Infarction.

In some cases, a geriatric and aging panel may be used to test an individual aged over approximately 40 years old, over approximately 50 years old, over approximately 60 years old, or over approximately 70 years old, e.g., 40, 45, 50, 55, 60, 65, 70, 80, 85, or 90 years old. In other cases, a geriatric panel may be used to test a younger individual, e.g., an individual who is younger than 40 years old. For example, an older or younger individual with a family or personal medical history of chronic disease(s), degenerative disease(s), abnormal lipid level(s), osteoarthritis, or any disease or condition provided herein may be tested with a geriatric and aging panel or subset thereof (see, e.g., FIG. 21B). In some cases, a geriatric and aging panel may be used to test an individual for a set of two or more risks, for example, such set may include one of the following sets of risks or predispositions: predisposition for certain reactions to medication (e.g., sensitivity to, metabolism of, adverse reactions to) and risk for osteoarthritis; risk for hearing loss and risk of stroke; risk of hearing loss and risk of coronary artery disease/myocardial infarction; risk for coronary artery disease/myocardial infarction and risk for osteoarthritis; risk of coronary artery disease/myocardial infarction and risk of bone mineral density abnormality/osteoporosis/osteoporotic fracture; risk of coronary artery disease/myocardial infarction and risk of osteoporosis; risk of coronary artery disease/myocardial infarction and risk of osteoporotic fracture; risk of coronary artery disease/myocardial infarction and risk of impaired visual acuity; risk of impaired visual acuity and risk of impaired hearing acuity; risk of stroke and risk of impaired visual acuity; or risk of stroke and risk of osteoarthritis.

An individual may choose to have the risk (as stated previously and as is applicable throughout, the term ‘risk’ can refer to either or both risk(s) for multifactorial phenotype(s) or carrier status of monogenic or polygenic phenotypes, such as carrier, non-carrier, affected, or likely affected by the phenotype(s)) or predisposition to other phenotypes, such as conditions, determined based on the initial panel results. For example, an individual may have a high risk of coronary artery disease and have reflex testing of an indicator of the effectiveness of and/or dose of statin to reduce risk of death or major cardiovascular events and/or reflex testing for the effectiveness of the antithrombotic activity of aspirin and/or reflex testing for the effectiveness of an oral antiplatelet agent, such as the platelet inhibitor clopidogrel or prasugrel or both, and/or reflex testing for sensitivity or resistance to warfarin and/or reflex testing to provide a genetically-tailored dose of warfarin.

An individual determined to have a high risk of osteoarthritis from the Golden Panel may choose to be tested with another panel, such as the Osteoarthritis Panel. Alternatively, the individual may have been tested with both panels initially. The risk or predisposition of an individual for phenotypes, such as at least 1, 2, or 3 of the following phenotypes: Osteoarthritis; Metabolism and/or Effectiveness and/or Choice and/or Dose and/or Sensitivity and/or Adverse Reactions to Medications used to Treat Arthritis; or Success of Joint Replacement as Treatment for Osteoarthritis.

An individual interested in the Golden Panel may also be interested in the Longevity Panel Alpha and/or Beta. Life insurance or disability insurance companies and issuers of life or disability insurance, or both, by also be interested in the Longevity Panel Alpha and/or Beta. The Longevity Panel Alpha and/or Beta, as with all panels, can also be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, the amnion, the amniotic sac, the blood of a pregnant female or via peripheral or central blood draw(s) from the pregnant female. The entire panel, or a subset, may be used. For example, individuals may select the Longevity Panel Alpha, which can be used to determine the risk or predisposition of an individual for phenotypes, such as at least 1, 2, 3, 4, or 5 of the following phenotypes: Longevity and/or Lifespan; Heart Disease (including but not limited to Coronary Artery Disease (CAD) and/or Myocardial Infarction); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Thrombophilia and/or Thromboembolic Disease; or Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections). A Longevity Panel Alpha can determine the risk or predisposition of a subset of the aforementioned phenotypes, such as at least 1, 2, 3, or 4 of the following phenotypes: Longevity and/or Lifespan; Heart Disease (including but not limited to Coronary Artery Disease (CAD) and/or Myocardial Infarction); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); or Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions.

Individuals may select the Longevity Panel Beta, which can be used to determine the risk or predisposition of an individual for phenotypes, such as at least 1, 2, 3, 4, 5, 6, 7, 8, 9, 10, or 11 of the following phenotypes: Longevity and/or Lifespan; Myocardial Infarction; Stroke (CVA); Arrhythmogenic Right Ventricular Cardiomyopathy; Wolff-Parkinson-White Syndrome; Malignant Hyperthermia; Lung Cancer; Breast Cancer; Colorectal Cancer; Human Immunodeficiency Virus (HIV) Infection Susceptibility; or Long QT Syndrome. A Longevity Panel Beta can determine the risk or predisposition of a subset of the aforementioned phenotypes, such as at least 1, 2, 3, or 4 of the following phenotypes: Longevity and/or Lifespan; Myocardial Infarction; Stroke (CVA); or Arrhythmogenic Right Ventricular Cardiomyopathy.

At times, an individual is tested for “phenotypes related to longevity”. “Phenotypes related to longevity” include any phenotype that is included in any of the following panels: Cardiovascular Panel Alpha, Cardiovascular Panel Beta, Heart Failure Panel, Coronary Artery Disease Panel, Myocardial Infarction Panel, Heartbeat/Arrhythmia Panel, Blood Panel, Dyslipidemia Panel, Lipid Level Panel, Blood Pressure Panel, Stroke Panel, Blood Flow, Thrombosis and Thromboembolism Panel, Longevity Panel Alpha, Longevity Panel Beta, Insurance Panel Alpha, Insurance Panel Beta; Research & Clinical Trial Panel; Exercise, Fitness and Athletic Training Panel, Sports Panel, Obesity Panel, Dietary, Nutrition & Weight Management Panel Alpha, Dietary, Nutrition & Weight Management Panel Beta, Executive Panel Alpha, Executive Panel Beta.

One of the ways The Research & Clinical Trial Panel may be utilized is to preserve wellness or increase longevity through research and clinical trials that are then able to utilize comprehensive genetic information. For example, The Research & Clinical Trial Panel may be utilized by governmental bodies (such as the United States Food and Drug Administration), researchers (such as at academic institutions) and/or companies (such as pharmaceutical companies) and may be helpful for basic research, bench research, translational research, clinical research and/or clinical trials for therapies, medications, treatments, medical devices, or any other substance or procedure that may prevent disease, treat disease, preserve wellness and/or maintain or increase longevity. For example, this panel may be utilized to aid in the development of medications used to treat or prevent cancer, heart disease, neurological diseases (such as Alzheimer's disease or Parkinson's disease), infectious diseases (such as HIV, malaria, tuberculosis, the common cold, influenza and cholera), rheumatologic diseases, and/or gastrointestinal diseases and may allow for these medications to be targeted at more specific demographics defined by their genetic profile at one or more genetic variants in their genome, allowing the research and the drugs to potentially have increased effectiveness, decreased toxicity, decreased adverse reactions, and more personalized dosing that will allow for more rapid onset of the medication's therapeutic effects with less side-effects or adverse drug reactions (thereby potentially increasing patient's adherence to the medication), thereby potentially increasing the wellness and/or longevity of the individual, such as a patient.

Women may be interested in specific panels, such as Women's Health Panel Alpha and/or Beta, or subset thereof, may be used to test a female human (e.g., woman, girl, female infant, female embryo, or female fetus) for her risk or predisposition for a particular disease or condition. For example, the Women's Health Panel Alpha, can be used to determine the risk or predisposition of an individual for phenotypes, such as at least 1, 2, 3, 4, 5, 6, 7, 8, 9, 10, 11, 12, or 13 of the following phenotypes: Female Fertility/Infertility and/or Spontaneous Abortion and/or Miscarriages and/or Reproduction System Abnormalities; Osteoporosis and/or Osteoporotic Fracture; Obesity or Leanness (including but not limited to Weight, BMI, Waist Circumference, Adiposity, and/or Fat Distribution); Heart Disease (including but not limited to Coronary Artery Disease (CAD) and/or Myocardial Infarction); Thrombophilia and/or Thromboembolic Disease; Cancer of Female Reproductive Organs (including but not limited to Breast Cancer, Ovarian Cancer, Cervical Cancer, Uterine Cancer, and/or Endometrial Cancer); Skin Cancer (Including Melanoma or Non-Melanoma Skin Cancer) and/or Sensitivity to UV Light; Lung Cancer; Alzheimer's Disease; Colorectal Cancer; Hypertension and/or Blood Pressure Level; Polycystic Ovary Syndrome; or Stroke (CVA). A Women's Health Panel Alpha can determine the risk or predisposition of a subset of the aforementioned phenotypes, such as at least 1, 2, 3, 4, 5, 6, 7, 8, or 9 of the following phenotypes: Female Fertility/Infertility and/or Spontaneous Abortion and/or Miscarriages and/or Reproduction System Abnormalities; Osteoporosis and/or Osteoporotic Fracture; Obesity or Leanness (including but not limited to Weight, BMI, Waist Circumference, Adiposity, and/or Fat Distribution); Heart Disease (including but not limited to Coronary Artery Disease (CAD) and/or Myocardial Infarction); Thrombophilia and/or Thromboembolic Disease; Cancer of Female Reproductive Organs (including but not limited to Breast Cancer, Ovarian Cancer, Cervical Cancer, Uterine Cancer, and/or Endometrial Cancer); Skin Cancer (Including Melanoma or Non-Melanoma Skin Cancer) and/or Sensitivity to UV Light; Lung Cancer; or Alzheimer's Disease.

Individuals may select the Women's Health Panel Beta, which can be used to determine the risk or predisposition of an individual for phenotypes, such as at least 1, 2, 3, 4, 5, 6, 7, 8, 9, 10, or 11 of the following phenotypes: Myocardial Infarction; Breast Cancer; Osteoporosis and/or Osteoporotic Fracture; Alzheimer's Disease; Thrombophilia and/or Thromboembolic Disease; Arrhythmogenic Right Ventricular Cardiomyopathy; Premenstrual Dysphoric Disorder; Hypertrophic Cardiomyopathy; Obesity or Leanness (including but not limited to Weight, BMI, Waist Circumference, Adiposity, and/or Fat Distribution); Skin Cancer (Including Melanoma or Non-Melanoma Skin Cancer) and/or Sensitivity to UV Light; or Lung Cancer. A Women's Health Panel Beta can determine the risk or predisposition of a subset of the aforementioned phenotypes, such as at least 1, 2, 3, 4, 5, or 6 of the following phenotypes: Myocardial Infarction; Breast Cancer; Osteoporosis and/or Osteoporotic Fracture; Alzheimer's Disease; Thrombophilia and/or Thromboembolic Disease; or Arrhythmogenic Right Ventricular Cardiomyopathy.

For example, a women's health panel may be used to test a female human for two or more risks including her risk of cardiovascular disease (e.g., coronary artery disease and/or myocardial infarction) and her risk of a cancer of the reproductive system (e.g., breast and/or ovarian cancer). In some cases, a women's health panel may be used to test a female human for a set of two or more risks, for example, such set may include one of the following sets of risks: her risk of osteoporosis and her risk of a breast cancer; her risk of obesity and her risk of breast cancer; her risk of thrombophilia/thromboembolic disease and her risk of breast cancer; her risk of Alzheimer's Disease and her risk of breast cancer; her risk of reproductive abnormalities (e.g., fertility, miscarriage) and her risk of breast cancer; or her risk of osteoporosis and her risk of Alzheimer's Disease. In other cases, other combinations of conditions related to women's health, or conditions listed in the Women's Health Panel, may be tested. In some cases, if a woman has a high risk for a certain condition, reflex testing may be performed. For example, if a woman has a high risk of osteoporosis, testing for indicators of how specific diets and/or caffeine influence osteoporosis risk can be performed.

A female human (e.g., woman, girl, female infant, female developing embryo/fetus, etc.) may also be tested using a gynecology panel (see, e.g. the Gynecology Panel) or subset thereof. For example, the panel can be used to determine the risk or predisposition of an individual for phenotypes, such as at least 1, 2, 3, 4, 5, 6, 7, or 8 of the following phenotypes: Breast Cancer; Thrombophilia and/or Thromboembolic Disease; Premenstrual Dysphoric Disorder; Human Papillomavirus (HPV) Susceptibility; Ovarian Abnormalities and/or Failure; Iron Deficiency Anemia in Menstruating Women; Human Immunodeficiency Virus (HIV) Infection Susceptibility; or Ovarian Cancer. A Gynecology Panel can determine the risk or predisposition of a subset of the aforementioned phenotypes, such as at least 1, 2, or 3 of the following phenotypes: Breast Cancer; Thrombophilia and/or Thromboembolic Disease; or Premenstrual Dysphoric Disorder.

In some cases, a gynecology panel may be used to test a woman or girl with a medical history of one or more of the following: iron deficiency, anemia, Human Papilloma Virus (HPV) positive, breast mass (e.g., breast mass discovered by physical exam or by radiological exam) or other condition. A woman or girl with a family or medical history of a cancer of the reproductive system (e.g., breast, ovary, cervix, uterus, endometrium), may also be tested using the Gynecology Panel or the Women's Health Panel. For example, a woman may have a mother with breast cancer and may be tested with the Gynecology Panel. The entire panel may be selected, or a subset.

A male (e.g., man, boy, male infant, male developing embryo/fetus, etc.) may be interested in the Men's Health Panel Alpha and/or Beta. For example, individuals may select the Men's Health Panel Alpha, which can be used to determine the risk or predisposition of an individual for phenotypes, such as at least 1, 2, 3, 4, 5, 6, 7, 8, 9, 10, 11, or 12 of the following phenotypes: Male Fertility/Infertility (including but not limited to Abnormal Sperm Count and/or Abnormal Sperm Motility and/or Abnormal Sperm Morphology); Androgenic Alopecia; Heart Disease (including but not limited to Coronary Artery Disease (CAD) and/or Myocardial Infarction); Thrombophilia and/or Thromboembolic Disease; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Cancer of Male Reproductive Organs including but not limited to Prostate Cancer and/or Testicular Cancer and/or Germ Cell Tumor; Skin Cancer (Including Melanoma or Non-Melanoma Skin Cancer) and/or Sensitivity to UV Light; Lung Cancer; Colorectal Cancer; Alzheimer's Disease; Hypertension and/or Blood Pressure Level; or Stroke (CVA). A Men's Health Panel Alpha can determine the risk or predisposition of a subset of the aforementioned phenotypes, such as at least 1, 2, 3, 4, 5, 6, 7, 9 or 10 of the following phenotypes: Male Fertility/Infertility (including but not limited to Abnormal Sperm Count and/or Abnormal Sperm Motility and/or Abnormal Sperm Morphology); Androgenic Alopecia; Heart Disease (including but not limited to Coronary Artery Disease (CAD) and/or Myocardial Infarction); Thrombophilia and/or Thromboembolic Disease; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Cancer of Male Reproductive Organs including but not limited to Prostate Cancer and/or Testicular Cancer and/or Germ Cell Tumor; Skin Cancer (Including Melanoma or Non-Melanoma Skin Cancer) and/or Sensitivity to UV Light; Lung Cancer; Colorectal Cancer; or Alzheimer's Disease.

Individuals may select the Men's Health Panel Beta, which can be used to determine the risk or predisposition of an individual for phenotypes, such as at least 1, 2, 3, 4, 5, 6, 7, 8, 9, or 10 of the following phenotypes: Myocardial Infarction; Melanoma; Colorectal Cancer; Prostate Cancer; Androgenic Alopecia; Erectile Dysfunction Medication Treatment Effectiveness and/or Sensitivity (including but not limited to inhibitors of cGMP phosphodiesterase type 5); Thrombophilia and/or Thromboembolic Disease; Lumber Disc Disease; Alzheimer's Disease; or Arrhythmogenic Right Ventricular Cardiomyopathy. A Men's Health Panel Beta can determine the risk or predisposition of a subset of the aforementioned phenotypes, such as at least 1, 2, 3, 4, or 5 of the following phenotypes: Myocardial Infarction Melanoma; Colorectal Cancer; Prostate Cancer; or Androgenic Alopecia.

For example, a male human may be tested for at least two risks, including his risk of cardiovascular disease (e.g., coronary artery disease and/or myocardial infarction) and his risk of prostate cancer. In some cases, a men's health panel may be used to genetically test a male human for a set of two or more phenotypes or risks, for example, such set may include one of the following sets of risks: his risk of prostate cancer and his risk of Alzheimer's Disease; his risk of coronary artery disease and his risk of male-pattern baldness; his risk of decreased fertility and his risk of prostate cancer; his risk of prostate cancer and his risk of male-pattern baldness; or his risk of prostate cancer and his risk of colorectal cancer. In other cases, other combinations of phenotypes, such as conditions, related to men's health, or phenotypes, such as conditions, listed in Men's Health Panel, may be tested. If a male individual is determined to have a high risk or predisposition to a phenotype, such as a condition, a reflex condition may be tested. For example, if a male individual has a high risk of prostate cancer, reflex testing for indicators of prognosis and aggressiveness of prostate cancer, indicators of survival rate from prostate cancer, indicators of the effectiveness, metabolism, choice, dose, adverse reaction of medications to treat prostate cancer, and radiosusceptibility and/or residual DNA damage level from radiation to treat prostate cancer can also be determined for the male individual.

Individuals who want a thorough examination of their genome for possible genetic variants associated with sudden death (such as due to cardiac arrhythmias or myocardial infarction) or chronic and debilitating diseases that increase in risk with aging (such as Alzheimer's Disease) and/or are concerned with diseases or traits that may affect work performance (such as Attention Deficit Hyperactivity Disorder) and/or are concerned about their own security or corporate security or need to confirm/verify their identity (Universal Identifier, including, or not, Blood Group) and/or longevity (such as to provide an approximation of lifespan or length of life or age at death), as well as individuals with high-stress lifestyles, health conscious, or all or part of the above, may be tested with an executive panel, such as the Executive Panel Alpha and/or Beta, or subset thereof. For example, the Executive Health Panel Alpha can determine the risk or predisposition of an individual for phenotypes, such as at least 1, 2, 3, 4, 5, 6, 7, 8, 9, 10, 11, 12, 13, 14, 15, 16, or 17 of the following phenotypes: Universal Identifier and Blood Group; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome); Heart Disease (including but not limited to Coronary Artery Disease (CAD) and/or Myocardial Infarction and/or Cardiomyopathy); Thrombophilia and/or Thromboembolic Disease; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions (such as of the cancers listed herein); Stroke (CVA); Alzheimer's Disease; Osteoarthritis; Peptic Ulcer Disease; Longevity and/or Lifespan; Effect of Stimulant(s) on Cognition; or Caffeine Metabolism (including but not limited to Caffeine Consumption's Effect on Sleep); Androgenic Alopecia; Genetic Age and Effectiveness of Current and/or Past Exercise Regimens; Attention Deficit Hyperactivity Disorder; Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections).

An Executive Panel Alpha can determine the risk or predisposition of a subset of the aforementioned phenotypes, such as at least 1, 2, 3, 4, 5, 6, 7 or 8 of the following phenotypes: Universal Identifier and Blood Group; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome); Heart Disease (including but not limited to Coronary Artery Disease (CAD) and/or Myocardial Infarction and/or Cardiomyopathy); Thrombophilia and/or Thromboembolic Disease; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions (such as of the cancers listed herein); Stroke (CVA); or Alzheimer's Disease.

Individuals may select the Executive Panel Beta, which can be used to determine the risk or predisposition of an individual for phenotypes, such as at least 1, 2, 3, 4, 5, 6, 7, 8, 9, 10, 11, 12, 13, or 14 of the following phenotypes: Coronary Artery Disease (CAD); Myocardial Infarction; Arrhythmogenic Right Ventricular Cardiomyopathy; Hypertrophic Cardiomyopathy; Wolff-Parkinson-White Syndrome; Caffeine Metabolism (including but not limited to Caffeine Consumption's Effect on Sleep); Melanoma; Traveler's Diarrhea Susceptibility; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Stroke (CVA); Alzheimer's Disease; Dyslipidemia (Including Total Cholesterol and/or LDL Cholesterol and/or HDL Cholesterol and/or Triglycerides and/or Chylomicrons); Macular Degeneration; or Non-melanoma Skin Cancer. An Executive Panel Beta can determine the risk or predisposition of a subset of the aforementioned phenotypes, such as at least 1, 2, 3, 4, 5, 6, 7, or 8 of the following phenotypes: Coronary Artery Disease (CAD); Myocardial Infarction; Arrhythmogenic Right Ventricular Cardiomyopathy; Hypertrophic Cardiomyopathy; Wolff-Parkinson-White Syndrome; Caffeine Metabolism (including but not limited to Caffeine Consumption's Effect on Sleep); or Melanoma; Traveler's Diarrhea Susceptibility.

In some cases, an Executive Health Panel may be used to test an individual for a set of two or more risks (or predisposition) or carrier status, for example, such set may include one of the following sets of risks: his or her risk for coronary artery disease/myocardial infarction and his or her risk for peptic ulcer disease; his or her risk for coronary artery disease/myocardial infarction and his or her risk for sudden death; his or her predisposition for a Universal Identifier (e.g., his or her blood group or other identifying characteristic) and his or her risk for coronary artery disease/myocardial infarction; his or her risk for stroke and his or her risk for peptic ulcer disease; his or her risk for cancer and his or her risk for peptic ulcer disease; his or her risk for cancer and his or her risk for stroke; his or her predisposition for a stimulant having a positive or negative effect on cognition and his or her risk of coronary artery disease and/or myocardial infarction; his or her risk of addiction and his or her risk of stroke or other set of risks or predispositions, including those shown in the Executive Panel.

If an individual is at a high risk or found to have a predisposition for a certain phenotype, such as a condition, or are found to be carriers of a phenotype (such as if they carry a monogenic phenotype or if they are affected by a monogenic phenotype or are likely affected by a monogenic phenotype), or if they are diagnosed with a phenotype or if a diagnosis is being considered for a phenotype, reflex testing for another phenotype, such as a condition, may be performed. For example, testing an individual with the Executive panels shows that the individual has a high risk for peptic ulcer disease and reflex testing can be performed for indicators of metabolism, dosing, and sensitivity to medications used to treat peptic ulcer disease, risk of esophageal cancer due to gastroesophageal reflux disease (GERD), and risk of gastric cancer.

An exercise, fitness and athletic training panel, see, e.g., the Exercise, Fitness, and Athletic Training Panel, may be used to test the predisposition of an individual to develop, obtain or be capable of obtaining, a certain level of fitness. In some cases, an exercise, fitness and athletic training panel may be used to help develop a genetically-tailored workout or fitness regimen or to optimize a workout or fitness regimen. Such panel may be useful for any person engaging in athletic activity, for example, amateur or professional athletes, children participating in athletics, individuals who athletically train or workout on their own or with an athletic trainer or instructor, such as at a fitness club or gym, pre-college or college athletes, and individuals that exercise or want to start to exercise in order to improve or maintain their health and/or to augment their aesthetics and/or to excel in a sport. Such panel may also be useful for individuals with a history of one or more of the following: fatigue with exercise, difficulty motivating to exercise, obesity, being overweight or underweight, diabetes mellitus, pre-diabetes mellitus, exercise intolerance, or concerned/worried about their health. Individuals who have previously had limited success from exercise or workouts in the past may also select such panel. Methods for analysis of genetic variants related for prediction of phenotypes associated with specific types of athletes, athletic predisposition, and athletic performance are provided herein. This includes helpful information to discern a specific physical exercise regimen for most efficient physical exercise as well as an exercise regimen and/or workout that is most likely to produce the greatest returns. Individuals can be predisposed (i.e. have a higher risk of performing optimally at and be genetically inclined towards) specific physical activity and, at the same time, be predisposed (i.e. have a higher risk of performing less optimally and be genetically inclined against) other specific types of physical activity. Physical activity may refer to athletic performance, elite athletic performance (meaning exceptional performance at specific type of physical activity that may be at the level of being able to compete at the university-level, semi-professional, professional, national, international, and/or Olympic level), sporting events, community athletics, fitness-related exercise, health-related physical exercise, fitness training, sports training, fitness club exercise, and recreational exercise. Physical activity may also apply to physical activities that an individual may have to perform for their work, profession, or occupation, such as a fitness instructor at a fitness center, a police officer in law enforcement, or Infantry in the military. Predisposition to physical activity may be observed in and applicable to children (including but not limited to ages 2-19) and adults (ages 20-100).

Specific physical (athletic) predisposition genetic testing and/or analysis allows for the creation of a genetically tailored exercise regimen, workout program, or athletic-event guidance that is focused upon one or more specific physical activities that the individual is genetically predisposed to and may also allow the individual to avoid or limit exposure to one or more specific physical activities that they are either not predisposed to or are predisposed against performing optimally. Following this approach, the individual may be able to increase adherence to a physical activity program and increase returns (e.g. increased fitness, increased exercise capacity, increased time until fatigability and/or until the individual has to stop the exercise, increased cardiovascular health, decreased cholesterol and/or LDL levels, increased HDL levels, decreased blood pressure, increased insulin sensitivity, decreased risk of diabetes mellitus, type II, decreased risk of cancer, decreased risk of macular degeneration, decreased risk of physical injury (such as muscle or bone injury), increased calorie expenditure and resulting increased weight loss primarily due to decreased adiposity throughout the body, increased leanness, increased muscle mass, increased muscle strength, better physical functioning with aging, increased confidence in athletic ability and/or athletic performance, increased amount of self-confidence, augmented self-image, increased self-worth, decreased amount of aging, such as decreased amount of genetic aging including but not limited to decreased amount of shortening of telomeres over time, and/or increased psychological reinforcement that the physical activity they are participating in is having desired benefits) as opposed to performing athletic activity that they are either not predisposed to or are predisposed against (genetically inclined to perform less optimally) from that physical exercise.

Individuals can be genetically predisposed to endurance-related physical activities or power-related physical activities. Endurance-related physical activities are less intense, longer in duration and may utilize slow-twitch muscle fibers and/or differences in either the oxygen sensing and/or oxygen utilization mechanisms of the body and/or differences in the regulation of the aerobic/anaerobic metabolic system. They consist of low to medium intensity physical activity for longer durations of time (such as equal to or greater than 20 minutes and as long as 10 hours or greater without any time or significant time to stop and/or rest either a specific muscle group or the entire body). Examples of endurance-related physical activities include long distance activities, such as running, rowing, kayaking, canoeing, cycling, marching, mountaineering, or skiing (e.g. cross country skiing) for about 20 minutes to 10 hours or more. Genetic variants may be associated with either a general predisposition to endurance-related physical activities or with a predisposition to elite endurance-related athletic performance (such as performance greater than 95% of the general population and/or university-level, semi-professional, professional, national, international and/or Olympic-level performance). Analysis of genetic variants that are associated with endurance-related physical activities may also predict that the individual is predisposed against power-related physical activities such that they may not be able to perform power-related physical activities as well and they may not see the same returns or benefits from power-related physical activities as they would from endurance-related physical activities. Examples of genetic variants that affect endurance-related physical activity associated phenotypes include variants in the ACE or ACE-I gene (angiotensin-I converting enzyme), the ACTN3 gene, and the EPAS1 gene.

Power-related physical activities are more intense, shorter in duration and may utilize fast-twitch muscle fibers and/or differences in either the oxygen sensing and/or oxygen utilization mechanisms of the body and/or differences in the regulation of the aerobic/anaerobic metabolic system. They consist of high intensity physical activity for shorter durations of time (such as less than 20 minutes and as short as a few seconds before significant time to stop and/or rest either a specific muscle group of the entire body). Examples of power-related physical activities include short distance (such as equal to or less than 600 meters) physical activity such as events that require sprinting, events that require running or swimming until 600 meters, gymnastics, soccer, volleyball, wrestling, down-hill skiing, tennis, boxing, archery, short-distance swimming, dashes, resistance training, weight training, weightlifting, and rapid assaults such as when law enforcement or military quickly moves into an area. Genetic variants may be associated with either a general predisposition to power-related physical activities or a predisposition to elite power-related athletic performance (such as performance greater than 95% of the general population and/or university-level, semi-professional, professional, national, international and/or Olympic-level performance). Analysis of genetic variants that are associated with power-related physical activities may predict that the individual is predisposed against endurance-related physical activities such that they may not be able to perform endurance-related physical activities as well and they may not see the same returns or benefits from endurance-related physical activities as they would from power-related physical activities. Examples of genetic variants that affect power-related physical activity associated phenotypes include variants in the ACE-I gene and the ACTN3 gene.

Individuals can be genetically predisposed to increased, normal, or decreased muscle strength and/or the amount of returns observed from resistance training and/or strength training, such as isometric strength training. Some individuals may be found to be predisposed to increased returns from strength training, normal returns from strength training, or diminished returns from strength training. Examples of genetic variants that affect muscle strength and/or the amount of returns observed from resistance training and/or strength training associated phenotypes include variants in the ACE-I gene, the Resistin gene, the Myostatin gene, and the ACTN3 gene.

Individuals who are trying to achieve a specific end-point, such as reduction in total cholesterol levels, reduction in LDL levels, increased in HDL levels, decreased blood pressure, increased self-confidence, increased self-worth, increased insulin sensitivity, decreased risk of cardiovascular disease, decreased risk of macular degeneration, decreased risk of cancer, or decreased risk of diabetes mellitus, type II or healthcare professionals, insurance agencies, governments, a third party who are attempting to achieve a specific end-point in an individual may also benefit from a genetically-tailored exercise regimen. The genetically tailored exercise regimen may allow these individuals to observe the greatest returns (achieving their desired end-point) for the least amount of physical effort. The greater returns may psychologically reinforce their exercise regimen and therefore further increase the time they spend exercising, leading to more persistent long-term achievement of the desired end-points. Instead of just stating “exercise more” the healthcare professional, trainers, insurance companies, government, a third party or the individual themselves will be about to know that their genetically tailored exercise regimen is personalized for them and based upon their genetic code, thereby reinforcing the importance of the information conveyed (the information goes from being generic and therefore having potentially low-impact to becoming personalized and having potentially high-impact upon motivating the individual to pay attention to and adhere to the exercise regimen). Without this information based on the individual's genetic profile, the healthcare professional and/or the individual may conclude after a few weeks of not observing any response (such as no increased sensitivity and/or improved glucose metabolism) that the exercise wasn't working and therefore they may stop exercising (as not seeing returns will not reinforce their motivation or desire to continue exercise) and/or start an alternative treatment, such as prescription medications. Evaluating these other approaches may involve information from additional genetic variants, such as via reflex testing and/or analysis, such as those that are involved in medication effectiveness, adverse reactions, and dosing (such as whether an individual is at increased risk for myopathy associated with statin that may be prescribed to attempt to decrease cholesterol levels), those that dictate which diets will produce the greatest or least amount of decrease of body fat and/or weight, and/or those that are involved in preventive strategies, such as omega-3 supplementation (and the effectiveness of and/or degree to which one or more omega-3 fatty acids are metabolized within the body). Examples of genetic variants that affect end-point achievement associated phenotypes including but not limited to those provided herein include variants in the FHL1 gene.

Methods for analysis of genetic variants related to predicted phenotypes related to specific types of athletes, athletic predisposition, and athletic performance are provided herein. This includes helpful information to discern a specific physical exercise regimen for most efficient physical exercise as well as an exercise regimen and/or workout that is most likely to produce the greatest returns. Individuals can be predisposed (i.e. have a higher risk of performing optimally at and be genetically inclined towards) specific physical activity and, at the same time, be predisposed (i.e. have a higher risk of performing less optimally and be genetically inclined against) other specific types of physical activity. Physical activity may refer to athletic performance, elite athletic performance (meaning exceptional performance at specific type of physical activity that may be at the level of being able to compete at the university-level, semi-professional, professional, national, international, and/or Olympic level), sporting events, community athletics, fitness-related exercise, health-related physical exercise, fitness training, sports training, fitness club exercise, and recreational exercise. Physical activity may also apply to physical activities that an individual may have to perform for their work, profession, or occupation, such as a fitness instructor at a fitness center, a police officer in law enforcement, or Infantry in the military. Predisposition to physical activity may be observed in and applicable to children (including but not limited to ages 2-19) and adults (ages 20-100).

Specific physical (athletic) predisposition genetic testing and/or analysis allows for the creation of a genetically tailored exercise regimen, workout program, or athletic-event guidance that is focused upon one or more specific physical activities that the individual is genetically predisposed to and may also allow the individual to avoid or limit exposure to one or more specific physical activities that they are either not predisposed to or are predisposed against performing optimally. Following this approach, the individual may be able to increase adherence to a physical activity program and increase returns (e.g. increased fitness, increased exercise capacity, increased time until fatigability and/or until the individual has to stop the exercise, increased cardiovascular health, decreased cholesterol and/or LDL levels, increased HDL levels, decreased blood pressure, increased insulin sensitivity, decreased risk of diabetes mellitus, type II, decreased risk of cancer, decreased risk of macular degeneration, decreased risk of physical injury (such as muscle or bone injury), increased calorie expenditure and resulting increased weight loss primarily due to decreased adiposity throughout the body, increased leanness, increased muscle mass, increased muscle strength, better physical functioning with aging, increased confidence in athletic ability and/or athletic performance, increased amount of self-confidence, augmented self-image, increased self-worth, decreased amount of aging, such as decreased amount of genetic aging including but not limited to decreased amount of shortening of telomeres over time, and/or increased psychological reinforcement that the physical activity they are participating in is having desired benefits) as opposed to performing athletic activity that they are either not predisposed to or are predisposed against (genetically inclined to perform less optimally) from that physical exercise.

Individuals can be genetically predisposed to endurance-related physical activities or power-related physical activities. Endurance-related physical activities are less intense, longer in duration and may utilize slow-twitch muscle fibers and/or differences in either the oxygen sensing and/or oxygen utilization mechanisms of the body and/or differences in the regulation of the aerobic/anaerobic metabolic system. They consist of low to medium intensity physical activity for longer durations of time (such as equal to or greater than 20 minutes and as long as 10 hours or greater without any time or significant time to stop and/or rest either a specific muscle group or the entire body). Examples of endurance-related physical activities include long distance activities, such as running, rowing, kayaking, canoeing, cycling, marching, mountaineering, or skiing (e.g. cross country skiing) for 20 minutes or longer and as long as 10 hours or more.

The entire panel may be selected, or a subset. For example, the Exercise, Fitness and Athletic Training Panel can determine the risk or predisposition of an individual for phenotypes, such as at least 1, 2, 3, 4, 5, 6, 7, 8, or 9 of the following phenotypes: Specific Physical Exercise Regimen for Most Efficient Physical Exercise (Greatest Returns from Physical Exercise); Obesity or Leanness (including but not limited to Weight, BMI, Waist Circumference, Adiposity, and/or Fat Distribution); Genetic Age and Effectiveness of Current and/or Past Exercise Regimens; Effects of Specific Diets and/or Exercise on Obesity and/or BMI and/or Adiposity and/or Bone Mineral Density and/or Lipid Levels and/or Insulin Resistance; Reduced Sleep Quality and Insomnia due to Caffeine Consumption; Whether or Not Testosterone Doping (Exogenous Testosterone Use) May Be Detected on Drug Screen (Urinary Testosterone/Epitestosterone Ratio Needed in order to Detect Testosterone Doping); Muscle Strength in Arms & Legs; Physical Functioning in Older Age; or Longevity and/or Lifespan. A Exercise, Fitness and Athletic Training Panel can determine the risk or predisposition of a subset of the aforementioned phenotypes, such as at least 1 or 2 of the following phenotypes: Specific Physical Exercise Regimen for Most Efficient Physical Exercise (Greatest Returns from Physical Exercise); or Obesity or Leanness (including but not limited to Weight, BMI, Waist Circumference, Adiposity, and/or Fat Distribution).

Individuals interested in the aforementioned panel may also be interested in the Sports Panel, which can be used to determine the risk or predisposition of an individual for phenotypes, such as at least 1, 2, 3, 4, 5, or 6 of the following phenotypes: Prognosis following Head Injury and/or Brain Injury (including but not limited to Cognitive Performance and/or Dementia and/or Alzheimer's Disease Susceptibility); Athletic Ability and/or Predisposition to Specific Sports (Including but not Limited to Athletic Performance and/or Exercise Tolerance and/or Athletic Predispositions and/or Optimal Exercise Regimen and/or Athletic Training Regimen); Hypertrophic Cardiomyopathy; Arrhythmogenic Right Ventricular Cardiomyopathy; Whether or Not Testosterone Doping (Exogenous Testosterone Use) May Be Detected on Drug Screen (Urinary Testosterone/Epitestosterone Ratio Needed in order to Detect Testosterone Doping); or Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury). A Sports Panel can determine the risk or predisposition of a subset of the aforementioned phenotypes, such as at least 1, 2, 3, or 4 of the following phenotypes: Prognosis following Head Injury and/or Brain Injury (including but not limited to Cognitive Performance and/or Dementia and/or Alzheimer's Disease Susceptibility); Athletic Ability and/or Predisposition to Specific Sports (Including but not Limited to Athletic Performance and/or Exercise Tolerance and/or Athletic Predispositions and/or Optimal Exercise Regimen and/or Athletic Training Regimen); Hypertrophic Cardiomyopathy; or Arrhythmogenic Right Ventricular Cardiomyopathy.

The Dietary, Nutrition and Weight Management Panel Alpha and/or Beta may be useful to individuals concerned about weight-management or food intake. Overweight, underweight, normal weight, or obese individuals, and/or individuals with low, normal, or high body mass index (BMI), and/or individuals with increased abdominal adiposity, may use the Dietary, Nutrition and Weight Management Panel in order to help design a genetically-tailored diet or nutritional program, or to help optimize such program. Individuals with a history of limited or no adherence or compliance or benefit (such as long-term or sustained weight reduction) with diet or nutritional programs also may be aided by such panel. The entire panel may be selected, or a subset. For example, the Dietary, Nutrition and Weight Management Panel Alpha can be used to determine the risk or predisposition of an individual for phenotypes, such as all or at least 1, 2, 3, 4, 5, 6, 7, 8, 9, 10, 11, 12, 13, or 14 of the following phenotypes: Obesity or Leanness (including but not limited to Weight, BMI, Waist Circumference, Adiposity, and/or Fat Distribution); Effects of Specific Diets on Weight and/or Obesity and/or BMI and/or Adiposity; Effects of Physical Exercise on Weight and/or Obesity and/or BMI and/or Adiposity; Physical Exercise Capacity (including but not limited to Fatigability with Physical Exercise); Specific Physical Exercise Regimens for Most Efficient Physical Exercise (Greatest Returns from Physical Exercise); Effects of Exercise on Lipid Levels; Effects of Specific Diets on Bone Mineral Density; Effects of Specific Diets on Lipid Levels; Effects of Specific Diets on Blood Pressure; Cancer risk with Consumption of Specific Foods and/or Beverages and/or Alcohol and/or Medications; Effects of Specific Foods (including but not limited to Fruits and/or Vegetables) and/or Beverage (including but not limited to Alcohol and/or Caffeine) Consumption on Heart Health and/or Risk of Atherosclerosis and/or Risk of Myocardial Infarction; Vitamin and/or Mineral and/or Element and/or Herbal and/or Nutritional Supplement Metabolism and/or Sensitivity and/or Dose and/or Choice and/or Adverse Reactions and/or Deficiency of; Taste Perception and/or Specific Food Preference; or Effectiveness of Appetite Suppressants including but not limited to Sibutramine for Weight Reduction. A Dietary, Nutrition & Weight Management Panel Alpha can determine the risk or predisposition of a subset of the aforementioned phenotypes, such as at least 1, 2, 3, 4, or 5 of the following phenotypes: Obesity or Leanness (including but not limited to Weight, BMI, Waist Circumference, Adiposity, and/or Fat Distribution) Effects of Specific Diets on Weight and/or Obesity and/or BMI and/or Adiposity; Effects of Physical Exercise on Weight and/or Obesity and/or BMI and/or Adiposity; Physical Exercise Capacity (including but not limited to Fatigability with Physical Exercise); or Specific Physical Exercise Regimens for Most Efficient Physical Exercise (Greatest Returns from Physical Exercise).

Individuals may select the Dietary, Nutrition & Weight Management Panel Beta, which can be used to determine the risk or predisposition of an individual for phenotypes, such as all or at least 1, 2, 3, 4, 5, 6, 7, 8, 9, or 10 of the following phenotypes: Obesity or Leanness (including but not limited to Weight, BMI, Waist Circumference, Adiposity, and/or Fat Distribution); Effects of Specific Diets on Weight and/or Obesity and/or BMI and/or Adiposity; Taste Perception and/or Specific Food Preference; Effectiveness of Appetite Suppressants including but not limited to Sibutramine for Weight Reduction; Association of Colorectal Cancer with Consumption of Specific Food (including but not limited to Dietary Red Meat); Effects of Specific Diets on Bone Mineral Density; Effects of Specific Diets on Lipid Levels; Effects of Specific Diets on Blood Pressure; Effects of Specific Foods (including but not limited to Fruits and/or Vegetables) and/or Beverage (including but not limited to Alcohol and/or Caffeine) Consumption on Heart Health and/or Risk of Atherosclerosis and/or Risk of Myocardial Infarction; Vitamin and/or Mineral and/or Element and/or Herbal and/or Nutritional Supplement Metabolism and/or Sensitivity and/or Dose and/or Choice and/or Adverse Reactions and/or Deficiency Thereof. A Dietary, Nutrition & Weight Management Panel Beta can determine the risk or predisposition of a subset of the aforementioned phenotypes, such as at least 1, 2, or 3 of the following phenotypes: Obesity or Leanness (including but not limited to Weight, BMI, Waist Circumference, Adiposity, and/or Fat Distribution); Effects of Specific Diets on Weight and/or Obesity and/or BMI and/or Adiposity; or Taste Perception and/or Specific Food Preference.

The study of genetic variants related to phenotypes affected by specific types of food and/or beverage consumption is called Nutrigenomics. Nutrigenomics refers to application of genetics to nutrition and nutrition-related phenotypes. A significant portion of an individual's metabolism is dictated by their genetic profile, including the metabolism of foods, beverages, and other ingested substances such as alcohol, illicit drugs, herbs, and toxins. Because an individual's metabolism is dictated by a combination of environment (such as a person's daily caloric intake) and genetics, a person's body mass index (BMI), weight, amount of body fat (adiposity), amount of leanness, and predisposition to being lean, overweight or obese both in-general and at specific points in an individuals life can be ascertained through genetic testing and analysis.

As an example, variations in the FTO gene are significantly correlated with both childhood and adult obesity, being responsible for approximately 13% of people who are overweight and 22% of people who are obese in the United States. Obesity due to this gene, in turn, has been shown to correlate with Type II Diabetes. These associations have been replicated in numerous studies and provide convincing evidence of a genetic determinants of BMI. Nutrigenetic information may provide insight into a diet that takes into account a person's genetic profile, thereby creating a genetically tailored diet. A genetically tailored diet has been shown to improve long-term weight management in individuals attempting to lose weight and/or maintain a certain weight, such as after losing weight. In some embodiments, the methods of the present invention provide for analyzing genetic variations that predispose to obesity only if the individual is a cigarette smoker such as for example the Apolipoprotein B gene. While some people actually smoke to try to lose weight, people with this genetic variation will actually have a higher BMI with smoking. Other genetic variants predispose a person to greater than 30% increased in BMI when they are between the ages of 20 to 50 years old, such as for example the catechol-O-methyltransferase (COMT) gene. Because of insight about this predisposition and through proper nutrition and increased exercise, the individual empower with this information may be able to avoid or greatly minimize significant weight gain when they are between the ages of 20-50 years old.

Genetic variants, such as for example variation in the Adenosine A2A Receptor Gene (ADORA2A), can impact how the body metabolizes caffeine and whether or not caffeine affects sleep patterns at night, such as the quality of sleep (such as if the individual feels rested the next day and/or if the person has normal REM cycles during sleep). People with caffeine sensitivity may experience reduced sleep quality and altered REM-cycle sleep after ingesting caffeine, even if the caffeine ingestion only occurs during the early morning. In these individuals, caffeine-induced changes in brain electrical activity during sleep resemble the alterations observed in patients with insomnia. Due to caffeine consumption, these people will actually feel more tired and run-down in the long term. The more tired they feel, the more caffeine they may drink and this can lead to an insidious cycle. This lack of sleep will not only decrease energy levels but will also increase stress, increase tiredness, decrease work productivity, and lead to the individual decreasing or stopping their fitness program due to tiredness, in-turn leading to an increased sedentary lifestyle and possibly higher risk of hypokinetic diseases, such as obesity.

Taste is also dictated by an individual's genetic profile and preference for certain types of food is dependent upon taste, and therefore dependent upon the genetic profile. For example, variants in TASR2 genes give rise to the genetically determined ability to taste phenylthiocarbamide is also associated with the ability to taste certain bitter foods, such as broccoli, Brussels sprouts, turnips, and kale (see, e.g. Kim, U.-k., E. Jorgenson, et al. (2003). “Positional Cloning of the Human Quantitative Trait Locus Underlying Taste Sensitivity to Phenylthiocarbamide.” Science 299(5610): 1221-1225; and Tepper, B. J. (1998). 6-n-Propylthiouracil: A Genetic Marker for Taste, with Implications for Food Preference and Dietary Habits.” The American Journal of Human Genetics 63(5): 1271-1276). Understanding or predicting a person's specific genetic profile will allow a genetically tailored nutrition program to avoid vegetables that the person may have a taste-aversion to, such as broccoli, Brussels sprouts, turnips, and kale and instead focus upon vegetables that the person is much more likely to find taste-favorable, such as green beans or potatoes.

Genetic variants also are important in determining the association between nutrition and disease risk. For example, association between caffeine and osteoporosis. Individuals who consume greater than 300 mg/day of caffeine (about three cups of coffee or about six cups of tea or about two ounces of chocolate or about one cup of chocolate or chocolate milk) experienced bone loss, such as in their spine, leading to osteoporosis with age (such as when the individual is older than 50 years old) (see, e.g. Rapuri, P. B., J. C. Gallagher, et al. (2007). “Caffeine decreases vitamin D receptor protein expression and 1.25(OH)2D3 stimulated alkaline phosphatase activity in human osteoblast cells.” The Journal of Steroid Biochemistry and Molecular Biology 103(3-5): 368-371).

Additional examples of nutrigenomic associated phenotypes include but are not limited to: Increased Vitamin Needs (such as B-vitamin Nutritional Supplementation, such as folate, riboflavin, cobalamin, and/or vitamin B6) In Order to Decrease Risk of Coronary Artery Disease; Increased Vitamin Needs (such as B-vitamin Nutritional Supplementation, such as folate, riboflavin, cobalamin, and/or vitamin B6) Not Needed In Order to Decrease Risk of Coronary Artery Disease; Increased Vitamin Needs (such as B-vitamin Nutritional Supplementation, such as folate, riboflavin, cobalamin, and/or vitamin B6) In Order to Decrease Elevated Homocysteine Level; Increased risk of Cancer (such as Prostate Cancer) with Low Vitamin Levels (such as Low Vitamin E Intake, such as ≦31.2 IU/day); Increased risk of Lower Circulating Levels of 25-dihydroxyvitamin D; Vitamin D-resistant Rickets; Vitamin D-dependent Rickets; Rickets Treatable with Vitamin D Supplementation; Vitamin K-dependent Coagulation Defect; Coagulation Defect Reversible with Oral Vitamin K1 Supplementation; Decreased risk of Cancer (such as Prostate Cancer) with Vitamin Supplementation (such as Vitamin E Supplementation, such as ≦31.2 IU/day); Vitamin B12-responsive Methylmalonic Aciduria due to Defect in Synthesis of Adenosylcobalamin, cblB Complementation Type; Protection against Neural Tube Defects (such as Spina Bifida) in Fetuses and/or Newborns of Mothers who Take Vitamin Supplements (such as B vitamins, such as Folate); Increased risk of Neural Tube Defects (such as Spina Bifida) in Fetuses and/or Newborns of Mothers who Did Not Use Vitamin Supplements (such as B vitamins, such as Folate); Protection against Cancer (such as Ovarian Cancer) with Vitamin Supplements (such as Multivitamin Supplements taken once a day); Increased risk of Cancer (such as Ovarian Cancer) if no Vitamin Supplements (such as Multivitamin Supplements taken once per day) are taken (or are taken less than once per day); Increased risk of Cancer (such as Colorectal Cancer) with Specific Diet (such as the Consumption of Red Meat, such as ≧one serving of red meat per day); No Increased risk of Cancer (such as Colorectal Cancer) due to Specific Diet (such as the Consumption of Red Meat); Increased risk of Colorectal Cancer from Exposure to Cigarette and/or Tobacco Smoke (Including but Not Limited to Environmental Exposure, also known as Second-hand Smoke Exposure and/or First-hand Exposure via the Individual themselves Smoking); No Increased risk of Colorectal Cancer from Exposure to Cigarette and/or Tobacco Smoke (Including but Not Limited to Environmental Exposure, also known as Second-hand Smoke Exposure and/or First-hand Exposure via the Individual themselves Smoking); Increase in HDL Levels with the Consumption of Alcohol (such as when equal to or greater than 25 g of alcohol is consumed per day); No Increase in HDL Levels with the Consumption of Alcohol (such as when equal to or greater than 25 g of alcohol is consumed per day); Decreased LDL/HDL (Improved Lipid Profile) Ratio in Response to Linoleic Acid-enriched, Low-cholesterol Diets; LDL/HDL Ratio Unchanged (Lipid Profile Unaffected) in Response to Linoleic Acid-enriched, Low-cholesterol Diets; Increased risk of Colorectal Adenoma with Alcohol Use (greater than or equal to 25 g of alcohol per day); No Increased risk of Colorectal Adenoma with Alcohol Use (greater than or equal to 25 g of alcohol per day); Highest Reduction of Body Fat (such as >5%) with Low Fat Diet (such as <10% of total calories from saturated fat and <300 mg cholesterol/day); Moderate Reduction of Body Fat (such as 2-5%) with Low Fat Diet (such as <10% of total calories from saturated fat and <300 mg cholesterol/day); No Substantial Reduction of Body Fat (such as <1%) with Low Fat Diet (such as <10% of total calories from saturated fat and <300 mg cholesterol/day); Highest Reduction of Body Fat (such as >5%) with Low Carbohydrate Diet (restrict carbohydrates to a level that induces a small level of ketosis and/or carbohydrate <12% total energy); Moderate Reduction of Body Fat (such as 2-5%) with Low Carbohydrate Diet (restrict carbohydrates to a level that induces a small level of ketosis and/or carbohydrate <12% total energy); No Substantial Reduction of Body Fat (such as <1%) with Low Carbohydrate Diet (restrict carbohydrates to a level that induces a small level of ketosis and/or carbohydrate <12% total energy); Lower Total Cholesterol with Specific Diet (such as High Long-chain n-3 Fatty Acid Intake (such as >0.32 g/day)); Lower LDL Cholesterol with Specific Diet (such as High Long-chain n-3 Fatty Acid Intake (such as >0.32 g/day)); Higher Total Cholesterol with Specific Diet (such as High Long-chain n-3 Fatty Acid Intake (such as >0.32 g/day)); Higher LDL Cholesterol with Specific Diet (such as High Long-chain n-3 Fatty Acid Intake (such as >0.32 g/day)); Lower Total Cholesterol with Specific Diet (such as High Long-chain n-6 Fatty Acid Intake (such as >7.99 g/day)); Lower LDL Cholesterol with Specific Diet (such as High Long-chain n-6 Fatty Acid Intake (such as >7.99 g/day)); Higher Total Cholesterol with Specific Diet (such as High Long-chain n-6 Fatty Acid Intake (such as >7.99 g/day)); Higher LDL Cholesterol with Specific Diet (such as High Long-chain n-6 Fatty Acid Intake (such as >7.99 g/day)); Lower Apolipoprotein C-III Levels with Specific Diet (such as High Polyunsaturated Fatty Acids Diet (such as >8%)); Lower Triglyceride Levels with Specific Diet (such as High Polyunsaturated Fatty Acids Diet (such as >8%)); Higher Apolipoprotein C-III Levels with Specific Diet (such as Low Polyunsaturated Fatty Acids Diet (such as <4%)); Higher Triglyceride Levels with Specific Diet (such as Low Polyunsaturated Fatty Acids Diet (such as <4%)); Increased risk of Obesity with a Westernized Diet (such as a High Fat Diet) as Compared to Traditional Japanese Diet (such as a Low Fat Diet); No Increased risk of Obesity with a Westernized Diet (such as a High Fat Diet) as Compared to Traditional Japanese Diet (such as a Low Fat Diet); Higher Bone Mineral Density with High Fat Diet; Higher Bone Mineral Density with Low Fat Diet; Lower Bone Mineral Density with High Fat Diet; Protection against Osteoporosis with High Fat Diet; Protection against Osteoporosis with Low Fat Diet; Increased Risk of Osteoporosis with High Fat Diet; Increased Insulin Resistance with Specific Diet (such as High Saturated Fatty Acid Diet, such as 38% Total Fat and 20% Saturated Fatty Acids); Decreased Insulin Resistance with Specific Diet (such as High in Monounsaturated Fatty Acid Diet, such as 38% total fat and 22% Monounsaturated Fatty Acid); Decreased Insulin Resistance with Specific Diet (such as Carbohydrate-rich Diet, such as 30% Total Fat and 55% Carbohydrate); Increased risk of Cancer (such as Lung Cancer) with Alcohol Consumption (such as ≧1 alcoholic drink per day); No Increased risk of Cancer (such as Lung Cancer) with Alcohol Consumption (such as ≧1 alcoholic drink per day); Protection against Myocardial Infarction with Alcohol Consumption (such as ≧1 alcoholic drink per day); No Protection against Myocardial Infarction with Alcohol Consumption (such as ≧1 alcoholic drink per day); Increased risk of Cancer with No or Light Alcohol Consumption (such as Squamous Cell Esophageal Cancer with Non-consumption or Low Consumption of Alcohol such as (<2 alcoholic drinks per week)); No Increased risk of Cancer with No or Light Alcohol Consumption (such as Squamous Cell Esophageal Cancer with Non-consumption or Light Consumption of Alcohol (such as <2 alcoholic drinks per week)); Increased risk of Cancer with Moderate to Heavy Alcohol Consumption (such as Squamous Cell Esophageal Cancer and/or Head with Medium to Heavy Consumption of Alcohol (such as ≧3 alcoholic drinks per week)); No Increased risk of Cancer with Moderate to Heavy Alcohol Consumption (such as Squamous Cell Esophageal Cancer and/or Head with Medium to Heavy Consumption of Alcohol (such as ≧3 alcoholic drinks per week)); Increased risk of Cancer with Moderate to Heavy Alcohol Consumption (such as Head & Neck Cancer with Medium to Heavy Consumption of Alcohol (such as ≧3 alcoholic drinks per week)); No Increased risk of Cancer with Moderate to Heavy Alcohol Consumption (such as Squamous Cell Head & Neck Cancer with Medium to Heavy Consumption of Alcohol (such as ≧3 alcoholic drinks per week)); Protection against Cancer with Moderate to Heavy Alcohol Consumption (such as Squamous Cell Head & Neck Cancer with Medium to Heavy Consumption of Alcohol (such as ≧3 alcoholic drinks per week)); Protection against Cancer with Light Alcohol Consumption (such as Squamous Cell Head & Neck Cancer with Light Consumption of Alcohol (such as 1-2 alcoholic drinks per week)); No Protection against Cancer with Light Alcohol Consumption (such as Squamous Cell Head & Neck Cancer with Light Consumption of Alcohol (such as 1-2 alcoholic drinks per week)); Lactose Tolerance or Intolerance; Indicator of whether or not a Specific Diet (such as Consumption of at least one serving per week of Cruciferous Vegetables) confers Protection against (Lowers Risk of) Heart Disease (such as Myocardial Infarction); Indicator of whether or not a Specific Diet (such as Consumption of at least one serving per week of Cruciferous Vegetables or Cabbage, Broccoli and/or Brussels Sprouts) confers Protection against (Lowers Risk of) Cancer (such as Lung Cancer); Specific Diet (such as a Low Fat Diet, such as a Dietary Approaches to Stop Hypertension (DASH)-style diet and/or <10% of total calories from saturated fat and <300 mg cholesterol/day) Produces Improved Lipid Profile (such as Decrease in Triglycerides and Total Cholesterol) in Individuals (such as the Obese); Specific Diet (such as a Low Fat Diet, such as a DASH-style diet and/or <10% of total calories from saturated fat and <300 mg cholesterol/day) Dose Not Produce Improved Lipid Profile (such as Decrease in Triglycerides and Total Cholesterol) in Individuals (such as the Obese); Decrease in Blood Pressure (such as a decrease of an average of 7 mm Hg systolic and 4 mm Hg diastolic) due to a Specific Diet (such as Sodium Restriction, such as less than 100 mmol per day and/or the DASH diet); No Decrease in Blood Pressure (such as a decrease of an average of 0-1 mm Hg systolic and 0-1 mm Hg Diastolic) due to a Specific Diet (such as Sodium Restriction, such as less than 100 mmol per day and/or the DASH diet); Increase of Intelligence (such as approximately 7 IQ points) with Breast Feeding; No Increased in Intelligence (such as approximately 0-1 IQ points) with Breast Feeding; Significant Reduction in Weight (such as 1-5% of weight) with Daily Exercise (such as at least 30 minutes of Cardiovascular Exercise with Average Heart Rate above 120 bpm); No Significant Reduction in Weight with Daily Exercise (such as at least 30 minutes of Cardiovascular Exercise with Average Heart Rate above 120 bpm); Reduced Levels of Satiety Following Meals (Such as Breakfast, Lunch, and/or Dinner); Normal Satiety Following Meals (Such as Breakfast, Lunch, and/or Dinner); Significant 25-year BMI Increase (such as an increased in BMI of between 5-15 kg/m2) starting in Youth (Such as the Age Range of between 6-15); No Significant 25-year BMI Increase (such as an increased in BMI of between 5-15 kg/m2) starting in Youth (Such as the Age Range of between 6-15); Increased BMI due to Added Weight (such as from Body Fat) from Increased Subcutaneous Fat and Increased Waist Circumference; Increased BMI due to Added Weight (such as from Body Fat) from Increased Hip Circumference; Increased Total Fat Mass of 2-week old Newborn; No Increase In Total Fat Mass of 2-week old Newborn; Increased Truncal Fat Mass of 2-week old Newborn; No Increase In Truncal Fat Mass of 2-week old Newborn; Increased Abdominal Fat Mass of 2-week old Newborn; No Increase In Abdominal Fat Mass of 2-week old Newborn; Increased BMI in Morbid Obesity (such as BMI over 45); Increased Waist-to-Hip Ratio (Increased Central Obesity); Decreased Waist-to-Hip Ratio (Decreased Central Obesity); Lower Thermic Effect of a Meal in Response to Fat Intake; Normal Thermic Effect of a Meal in Response to Fat Intake; Higher Thermic Effect of a Meal in Response to Fat Intake; Increased risk of Fenfluramine-associated Primary Pulmonary Hypertension; No Increased risk of Fenfluramine-associated Primary Pulmonary Hypertension; Increased risk of Self-imposed Severe Dietary Restriction (such as assessed by the Eating Attitudes Test) for Weight Loss Purposes; Protection against Self-imposed Severe Dietary Restriction (such as assessed by the Eating Attitudes Test) for Weight Loss Purposes; Increased risk of Bulimia Nervosa; Protection against Bulimia Nervosa; Earlier Age of Onset (Approximately 12-15 years old) of Weight Loss in Bulimia Nervosa; Later Age of Onset (Approximately 17-20 years old) of Weight Loss in Bulimia Nervosa; Increased risk of Anorexia Nervosa; and Protection against Anorexia Nervosa.

The Sexuality, Mate Selection, Relationships and Marriage/Divorce Panel may assist an individual in the selection of a mate or partner and/or may allow them to better understand their own sexuality such as sexual attraction and/or sexual identity. The Sexuality, Mate Selection, Relationships and Marriage/Divorce Panel may also be used along with the services provided by a dating service, matchmaker, dating web-site and the like, or by a therapist, psychologist, psychiatrist or endocrinologist. For example, when screening potential people as matches for a particular individual, a matchmaker may consider the individual's personal preferences, personality attributes, location, income, appearance, age, gender as well as information provided by the Sexuality, Mate Selection, Relationships and Marriage/Divorce Panel, e.g., the individual's predisposition for a specific level of pheromone perception or for matching people based on their genetic profiles which indicate a particular level of sexual attractiveness. The match-maker, dating service personnel, psychologist or other professional may compare the results from the individual's Sexuality, Mate Selection, Relationships and Marriage/Divorce Panel with those of other individual's known to the match-maker, dating service, or provider. In some cases, the matchmaker, dating service, psychologist or other service provider first matches the individual with a specific individual determined to be a good “match.” Then, as a later step in the screening process the matchmaker, dating service, or other service provider, may compare the results of the Sexuality, Mate Selection, Relationships and Marriage/Divorce Panel of the individual with his or her selected “match.” In other cases, the dating service, matchmaker, or other service provider compiles results from the Sexuality, Mate Selection, Relationships and Marriage/Divorce Panel for many or all of its clients in a central database; and the service may include such results as one of the criteria used to match clients. The entire panel may be selected, or a subset. For example, an individual may have analyzed/determined his or her risk or predisposition for all or at least 1, 2, 3, 4, or 5 of the following phenotypes: Sexual Attraction (Including but not Limited to Orgasm Potential and/or Sexual Responsiveness with Another Person); Pair Bonding (How Well People Bond with Their Partner); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Degree of Relationship Commitment and/or Divorce Potential; or Pheromone Perception. A Sexuality, Mate Selection, Relationships and Marriage/Divorce Panel can determine the risk or predisposition of a subset of the aforementioned phenotypes, such as at least 1, 2 or 3 of the following phenotypes: Sexual Attraction (Including but not Limited to Orgasm Potential and/or Sexual Responsiveness with Another Person); Pair Bonding (How Well People Bond with Their Partner); or Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance).

The Illness of Unknown Etiology Panel may be appropriate for an individual concerned about symptoms with unknown etiology or an individual concerned about a disease or condition that is ordinarily difficult to diagnose. Such panel may assist the individual, or the individual's physician or healthcare provider, or a hospital or clinic or insurance company, in determining whether the individual's symptom(s) can be attributed to a particular disease or condition. An individual with an abnormal test result (e.g., an abnormal erythrocyte sedimentation rate (ESR), an abnormal c-reactive protein (CRP), an abnormal Anti-Nuclear Antibody (ANA), or other abnormal test result) may be interested in testing for one or more of the conditions or diseases in the Illness of Unknown Etiology Panel. For example, an individual with an abnormal ANA may be tested for systemic lupus erythematosus (SLE). Individuals suffering from certain symptoms (e.g., fatigue, aches, pains, or fever) may also be interested in the Illness of Unknown Etiology Panel. Individuals with a family history of certain diseases (e.g., SLE, irritable bowel syndrome, multiple sclerosis) or medical history of certain diseases (e.g., fibromyalgia, irritable bowel syndrome) may also be tested with such panel. The entire panel, or a subset of the panel, may be used. For example, all of the following phenotypes, or a subset, such as at least 1, 2, 3, 4, 5, 6, 7, 8, 9, 10, or II of the following phenotypes: Chronic Fatigue; Fibromyalgia; Irritable Bowel Syndrome; Systemic Lupus Erythematosus (SLE); Inflammatory Bowel Disease (Including Crohn Disease and/or Ulcerative Colitis and/or Behçet's Syndrome); Celiac Disease; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Rare Diseases and/or Orphan Diseases and/or Metabolic Diseases and/or Syndromes; Sarcoidosis; Stressful Life Events causing Depressive Symptoms and/or Diagnosable Depression and/or Suicidality and/or Anxiety (including but not limited to Mental Vulnerability to Stress and/or Disease); or Depression and/or Seasonal Affective Disorder. An Illness of Unknown Etiology Panel can determine the risk or predisposition of a subset of the aforementioned phenotypes, such as at least 1, 2, 3, or 4 of the following phenotypes: Chronic Fatigue; Fibromyalgia; Irritable Bowel Syndrome; or Systemic Lupus Erythematosus (SLE).

The Military Panel Alpha and/or Beta may be useful to an applicant or candidate to the military or armed forces, a new recruit, a member of the military, or the military itself. For example, a branch of the military or armed forces may use the Military Panel to screen recruits; prospective personnel; current personnel; contractors, defense contractors, employees, or consultants; or an enemy, enemy combatant, or unlawful combatant. The Universal Identifier and Blood Group can be used to identify and/or confirm/verify, trace, or track the identity of both living (such as for security clearance reasons or if wounded on a battlefield) or dead military personnel, and may also be used to identify, confirm, verify, trace, or track the identity of a detainee, an enemy, potential enemy or suspected enemy, either alive or dead. The Universal Identifier as well as the Military Panel Alpha and/or Beta may also be useful to applicants or participants of programs or agencies that require one or more of the following: security, secrecy, physical conditioning, physical strength, psychological strength, training, aptitude, deceptiveness, memory, propensity for risk-taking behavior, agility, ability, base (such as minimal level) mental and/or psychological and/or intellectual and/or physical requirements or psychological conditioning, training, aptitude, ability, or base (such as minimal level) requirements, such as a space program, such as applicants to, employees of, consultants to, members of, or individuals associated with private or personal space flight, such as Virgin Galactic, Benson Space Company, Rocketplane Limited, Inc. EADS Astrium, Space Adventures, or XCOR Aerospace, or governmental space programs, such as the National Aeronautics and Space Administration (NASA), the European Space Agency (ESA), the Federal'noe kosmicheskoe agentstvo Rossii (Roskosmos), the Dokuritsu-gyōsei-hōjin Uchū Kōkū Kenkyū Kaihatsu Kikō (JAXA), the Sohnut HaHalal HaYisraelit (USA), the Natsional'ne kosmichne ahentstvo Ukrayiny (NSAU), the Bhāratīya Antariksh Anusandhān Sangatn (ISRO), and the China National Space Administration (CNSA). The Military Panel Alpha and/or Beta may also be useful to representative of or member of or part of the local, state, or federal government such as the military or armed forces or the police or other governmental agency or subagencies or related agencies such as the United States Secret Service, the Department of Defense (DoD), the Defense Advanced Research Projects Agency (DARPA), Department of Homeland Security (DHS), the Federal Bureau of Investigation (FBI), the National Security Agency (NSA), the Bureau of Alcohol, Tobacco, Firearms and Explosives (ATF), and the Central Intelligence Agency (CIA), the National Reconnaissance Office (NRO), the Joint Special Operations Command (JSOC), the Defense Intelligence Agency (DIA), the Bureau of Intelligence and Research (INR), the Office of Intelligence and Counterintelligence, the Drug Enforcement Administration (DEA), National Aeronautics and Space Administration (NASA), or international agencies such as North Atlantic Treaty Organization (NATO), the United Nations (UN) and the UN Security Council, or any other government or governmental agency of any country or collaboration of countries, such as the Secret Intelligence Service (SIS) and MI6, the Defense Intelligence Staff (DIS), the HaMossad leModi'in uleTafkidim Meyuhadim (Mossad), the Canadian Security Intelligence Service (CSIS), the Bundesnachrichtendienst (BND), the Naikaku Jōhō Chōsashitsu (Naichō), the Militaire Inlichtingen-en Veiligheidsdienst (MIVD), the Nasjonal sikkerhetsmyndighet (NSM), the Inter-Services Intelligence (ISI), the Federalnaya Sluzhba Bezopasnosti (FSB), the Australian Secret Intelligence Service (ASIS), the Departamento Administrativo de Seguridad (DAS), the Centro de Investigación y Seguridad Nacional (CISEN), the Agencia Federal de Investigacion (AFI), the Kor Risik DiRaja, the Sahmnakkhaogrong-hangshaat (NIA), the Re'asat Al Istikhabarat Al A'amah (GIP), the Security and Intelligence Division (SID), the Indian Space Research Organisation (ISRO), the National Directorate of Security (NDS), the Centro Nacional de Inteligencia (CNI), the National Security Bureau (NSB), the Directorate-General for External Security, the National Intelligence Service (NIS), the South African Department of Defense, the Canadian Department of National Defense, the Australian Department of Defense, or any other governmental organization or agency, as well as aerospace, defense, intelligence or advanced technology companies such as Lockheed Martin, Raytheon Company, or Northrop Grumman Corporation, or private security companies or private military companies (PMCs), such as Blackwater Worldwide, ArmorGroup International PLC, Hart Security, Military Professional Resources Inc. (MPRI), Pacific Architects and Engineers, or other third party. The entire panel may be selected, or a subset. For example, an individual may be tested, or may have analyzed/determined, all of the phenotypes listed in FIG. 16, or a subset, such as at least 1, 2, 3, 4, 5, 6, 7, 8, 9, 10, 11, 12, or 13, 14, 15, 16, 17, 18, 19, 20, or 21 of the following phenotypes: Universal Identifier and Blood Group; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Post Traumatic Stress Disorder Susceptibility; Sensitivity to and/or Adverse Reactions from Smallpox Vaccination; Sensitivity to Weapons of Mass Destruction; Visual Acuity (including but not limited to Visual Impairment and/or Myopia and/or Hyperopia and/or Glaucoma and/or Cataracts and/or Visuospatial/Perceptual Abilities and/or Color Perception and/or Color Blindness and/or Night Blindness and/or Age-related Maculopathy); Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury); Thrombophilia and/or Thromboembolic Disease; Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Effect of Stimulant(s) on Cognition; Stressful Life Events causing Depressive Symptoms and/or Diagnosable Depression and/or Suicidality and/or Anxiety (including but not limited to Mental Vulnerability to Stress and/or Disease); or Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections). A Military and Armed Forces Panel Alpha can be used to determine the risk or predisposition of an individual for all or a subset of the aforementioned phenotypes, such as at least 1, 2, 3, 4, 5, 6, or 7 of the following phenotypes: Universal Identifier and Blood Group; Intelligence and/or Intellectual Ability and/or Cognitive Ability (Including but not Limited to Intelligence Quotient, Verbal Memory, Working Memory, Visual Memory, Processing Speed, Attention, Recall, Verbal Language Skills, Cognitive Performance, Executive Functioning, Reward Learning, Abstract Reasoning Performance and/or Ability and Speed to Learn from Errors); Post Traumatic Stress Disorder Susceptibility; Sensitivity to and/or Adverse Reactions from Smallpox Vaccination; Sensitivity to Weapons of Mass Destruction; Visual Acuity (including but not limited to Visual Impairment and/or Myopia and/or Hyperopia and/or Glaucoma and/or Cataracts and/or Visuospatial/Perceptual Abilities and/or Color Perception and/or Color Blindness and/or Night Blindness and/or Age-related Maculopathy); or Athletic Ability and/or Predisposition to Specific Sports and/or Athletic Performance (Including but not Limited to Elite Athletic Performance and/or Exercise Tolerance and/or Athletic Predispositions and/or Optimal Exercise Regimen and/or Athletic Training Regimen) and/or Risk from Physical Activity (Including but not Limited to Prognosis and/or Cognitive Performance and/or Dementia and/or Alzheimer's Disease following Head Injury and/or Brain Injury).

The phenotypes and their associated genetic variants provided herein as useful to an applicant or candidate to the military or armed forces, a new recruit, a member of the military, or the military itself may in some cases provide specific data regarding risks of certain duties on an individual tested and analyzed by the methods of the present invention. For example, certain military duties such as for example infantrymen are more prone to exposure to weapons of mass destruction, such as for example chemical weapons such as for example nerve gas, than others. The methods of the present invention provide for testing and analyzing genetic variants in the PON1 gene which codes for serum paraoxonase. Genotypic variations have been identified in the paraoxonase gene, such as for example the variant rs662, which increase or decrease its ability to degrade or inactivate weapons of mass destruction such as for example chemical weapons, such as for example pesticide based chemical weapons including but not limited to organophosphates and/or nerve gas including but not limited to sarin nerve gas.

Alternatively, the Military and Armed Forces Panel Beta can be used alone, or in conjunction with the Alpha panel, or other panels disclosed herein, to determine the risk or predisposition of an individual for all of the phenotypes listed in FIG. 17, or a subset, such as at least 1, 2, 3, 4, 5, 6, 7, 8, 9, or 10 of the following phenotypes: Universal Identifier; Post Traumatic Stress Disorder Susceptibility; Specific Physical Exercise Regimen for Most Efficient Physical Exercise (Greatest Returns from Physical Exercise); Thrombophilia and/or Thromboembolic Disease; Violent Behavior; Noise-induced Hearing Impairment and/or Hearing Loss; Effect of Stimulant(s) on Cognition; Stressful Life Events causing Depressive Symptoms and/or Diagnosable Depression and/or Suicidality and/or Anxiety (including but not limited to Mental Vulnerability to Stress and/or Disease); Malaria Susceptibility; or Arrhythmogenic Right Ventricular Cardiomyopathy. A Military and Armed Forces Panel Beta can determine the risk or predisposition of an individual for all or a subset of the aforementioned phenotypes, such as at least 1, 2, 3, or 4 of the following phenotypes: Universal Identifier; Post Traumatic Stress Disorder Susceptibility; Specific Physical Exercise Regimen for Most Efficient Physical Exercise (Greatest Returns from Physical Exercise); or Thrombophilia and/or Thromboembolic Disease.

Military, government officials, law enforcement, investigative personnel and others may select the Law Enforcement/Forensic/Investigative Panel, which can be used to determine the risk or predisposition of an individual for all of the phenotypes listed in FIG. 18, or a subset, such as at least 1, 2, 3, 4, 5, 6, 7, or 8 of the following phenotypes: Universal Identifier/Identity Testing; Blood Group; Physical Traits (Including but not Limited to Ethnicity and/or Eye Color and/or Skin Color and/or Skin Pigmentation and/or UV Sensitivity and/or Tanning Response to Sunlight and/or Freckling and/or Mole Count and/or Hair Color and/or Hair Thickness and/or Androgenic Alopecia); Lineage and/or Ancestry Information; Height and/or Weight (Including but not Limited to Weight, BMI, Obesity, Leanness, Waist Circumference, Adiposity, and Fat Distribution); Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed (such as express anger outwardly and/or aggressively or inwardly and/or calmly and/or controlled), Novelty Seeking Behavior, and/or Harm Avoidance); Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders); or Age (Including but not Limited to Age Range and/or Approximate or Exact Age). A Law Enforcement/Forensic/Investigative Panel can determine the risk or predisposition of a subset of the aforementioned phenotypes, such as at least 1, 2, 3, or 4 of the following phenotypes: Universal Identifier/Identity Testing; Blood Group; Physical Traits (Including but not Limited to Ethnicity and/or Eye Color and/or Skin Color and/or Skin Pigmentation and/or UV Sensitivity and/or Tanning Response to Sunlight and/or Freckling and/or Mole Count and/or Hair Color and/or Hair Thickness and/or Androgenic Alopecia); or Lineage and/or Ancestry Information.

The Law Enforcement/Forensic/Investigative Panel can be used to identify unknown individuals or to help prevent or solve a crime. A sample from the individual (such as a suspect, a victim, or someone that wants to be protected and have security, such as a fetus, newborn, child or government official) can be obtained and a genetic profile generated with the Forensic Panel. The entire panel may be used, or a subset of conditions. The panel or conditions determined can be used for forensic molecular photofitting and used to identify characteristics of the individual, aiding in the identification of the individual. The individual may be dead or unconscious and thus unable to provide self-identification. The individual may be unable to provide identification for other reasons such as disorientation, hostility, or intoxication (including alcohol and drug-induced intoxication). The individual may be a victim of a crime, war, or a natural disaster, such as flooding, earthquakes, hurricane, and the like. The individual may also not be able to self-identify because of amnesia or brain injury. The individual may also be suspected of committing a crime or the individual may already be currently or previously arrested or currently or previously incarcerated or currently or previously on parole. In other cases, a biological sample may be used to obtain genetic material for genetic testing and may be tested in order to identify a suspect or potential suspect of a crime or a victim of a crime. For example, human tissue discovered at the scene of a crime may be tested using The Law Enforcement/Forensic/Investigative Panel in order to determine certain identifying characteristics (e.g., universal identifier, blood group, ancestry, ethnicity, skin tone, physical and morphological traits, height, weight, body habitus, eye color, hair color, hair thickness, androgenic alopecia, freckle count, mole count, visual acuity (such as myopia and/or likely to wear glasses or contact lenses), medications likely to be needed based by the individual, personality, psychiatric illness, or other phenotype(s) as listed in FIG. 18). The identifying characteristics may then be compared to the identifying information of a victim, a suspect or potential suspect. For example, all or a subset of the results of The Law Enforcement/Forensic/Investigative Panel may be compared with information in a central database or with characteristics of a specific suspect. The identifying information may be genetic variants, such as polymorphisms, or patterns of genetic variants, such as polymorphisms, or they may be the traits themselves. For example, if The Law Enforcement/Forensic/Investigative Panel identifies, or helps to identify, the human tissue as being derived from an adult Caucasian male approximately 30-45 years old of average height with poor eyesight, red hair and balding, blue eyes, pale skin with significant freckling, and who is most likely obese, such information may aid the generation of a description or portrait of the victim (such as if the body is missing) or of the suspect or may otherwise aid the apprehension of, or investigation of, a suspect of a crime. Utilizing phenotype information generated from the analysis of genotypic information from the genetic testing of tissue samples, a composite of the individual the tissue sample came from can be created, such as a written description, an auditory description, or a visual description such as representing the image of the individual as a printed picture, on a monitor or screen, on a handheld device such as a PDA or smartphone, or as a holographic image. In another example, if The Law Enforcement/Forensic/Investigative Panel analysis results correlate the human tissue with a particular blood type or with one or more particular genetic variant(s), such as polymorphism(s), pattern, such information may be compared to similar identifying information of individuals (e.g., convicted felons) stored in a governmental or agency or central database or other database of individuals. In another example, the Law Enforcement/Forensic/Investigative Panel may be useful to aid the prosecution or defense of an individual accused of committing a crime, or to absolve an individual of a crime, or in proceedings to overturn a prior conviction of an individual.

The Death/Autopsy Panel can also be used, for example, by family members of the individual, or for forensic or investigative purposes, such as by the local government, state government, the federal government, the armed forces, a hospital, an insurance company (such as a life insurance company), a coroner, a medical examiner, or an archaeologist, as described above, for example. This panel may be useful to help investigate the potential cause of death of someone who has recently died or who died a long time ago, such as if the cause of death is unknown or only suspected, or if the cause of death is questioned. This panel may also be useful to store a genetic sample and genetic code of the deceased, so that future genetic analysis can be conducted, if necessary, such as by the deceased's relatives, children or grandchildren or future generations. The panel can be used to determine phenotypes, such as at least 1, 2, 3, 4, 5, 6, 7, 8, 9, 10, 11, 12, 13, 14, or 15 of the following phenotypes: Wolff-Parkinson-White Syndrome; Long QT Syndrome; Arrhythmogenic Right Ventricular Cardiomyopathy; Brugada Syndrome; Ventricular Fibrillation; Ventricular Tachycardia; Sudden Infant Death Syndrome; Heart Block; Atrial Fibrillation; Drug-induced Long QT Syndrome; Drug-induced Torsade de Pointes; Thrombophilia and/or Thromboembolic Disease; Myocardial Infarction; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); or Hypertrophic Cardiomyopathy. A Death/Autopsy Panel can determine the risk or predisposition of a subset of the aforementioned phenotypes, such as at least 1, 2, 3, 4, 5, 6, or 7 of the following phenotypes: Wolff-Parkinson-White Syndrome; Long QT Syndrome; Arrhythmogenic Right Ventricular Cardiomyopathy; Brugada Syndrome; Ventricular Fibrillation; Ventricular Tachycardia; or Sudden Infant Death Syndrome.

Law enforcement officials, government, military, and such may also be interested in the Incarceration Panel, which can be used to determine the risk or predisposition of an individual for phenotypes, such as at least 1, 2, 3, 4, 5, 6, 7, or 8 of the following phenotypes: Universal Identifier and Blood Group; Violent Behavior; Human Immunodeficiency Virus (HIV) Infection Susceptibility or Resistance; Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders); Stressful Life Events causing Depressive Symptoms and/or Diagnosable Depression and/or Suicidality and/or Anxiety (including but not limited to Mental Vulnerability to Stress and/or Disease); Tendency to Experience Unprovoked Anger; or Drug Metabolism and/or Choice and/or Sensitivity and/or Adverse Reactions and/or Dosing (Including Pharmacogenomic Analysis for all Pharmaceuticals). An Incarceration Panel can determine the risk or predisposition of a subset of the aforementioned phenotypes, such as at least 1, 2, or 3 of the following phenotypes: Universal Identifier and Blood Group; Violent Behavior; or Human Immunodeficiency Virus (HIV) Infection Susceptibility or Resistance. This panel may be useful in discerning the risk associated with numerous individuals living within close living quarters, such as the infectious disease susceptibility and psychiatric diseases.

Law enforcement officials, government (and governmental agencies, such as the Food and Drug Administration (FDA) and international agencies, such as the European Union (EU) and the World Health Organization (WHO)), military, and other interested individuals, such as medical researchers, medical centers, hospitals, clinics, doctors, pharmaceutical companies, biotechnology companies, healthcare companies, and the such, may also be interested in the Pathology & Tissue Repository Panel, which can be used to determine the risk or predisposition of an individual for phenotypes, such as at least 1, 2, 3, 4, or 5 of the following phenotypes: Universal Identifier; Lineage and/or Ancestry Information; Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; or Heart Disease (including but not limited to Coronary Artery Disease (CAD) and/or Myocardial Infarction). A Pathology & Tissue Repository Panel can determine the risk or predisposition of a subset of the aforementioned phenotypes, such as at least 1, 2, or 3 of the following phenotypes: Universal Identifier; Lineage and/or Ancestry Information; or Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea). This panel may applied to tissue specimens that are stored at a hospital, medical center, research institution, biotechnology company, pharmaceutical company or during a research trial or clinical trial, or in a tissue repository, such as when tissue is stored within formalin or paraffin or any other substance, for preservation. This panel provides a universal identification for the tissue as well as a complete pharmacogenomic profile of this tissue specimen. This may aid in pharmaceutical companies or other researchers in choosing specific individuals or tissue samples to utilize during research or during clinical trials.

Medical researchers, government (and governmental agencies, such as the Food and Drug Administration (FDA), and international agencies, such as the European Union (EU) and the World Health Organization (WHO)), medical centers, hospitals, clinics, government agencies, doctors, pharmaceutical companies, biotechnology companies, healthcare companies and the such may also be interested in the Research & Clinical Trial Panel, which can be used to determine the risk or predisposition of an individual for phenotypes, such as at least 1, 2, 3, 4, 5, 6, 7, 8, 9, 10, II of the following phenotypes: Medication Metabolism and/or Adverse Reactions to Medications (Including but not Limited to Full Pharmacogenomics Analysis, Medication Dosing and/or Allergies and/or Choice of Medications and/or Medication Side Effects and/or Adverse Drug Reactions and/or Medication Interactions); Cardiac Arrhythmia and/or Cardiac Conduction Abnormality (Including but Not Limited to Atrial Fibrillation, Ventricular Fibrillation, Re-entry Arrhythmias, Wolff-Parkinson-White Syndrome, Arrhythmogenic Right Ventricular Dysplasia, Hypertrophic Cardiomyopathy, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Universal Identifier/Identity Testing; Ethnicity and/or Lineage and/or Ancestry Information; Blood Group; Vitamin and/or Mineral and/or Element and/or Herbal and/or Nutritional Supplement Metabolism and/or Sensitivity and/or Dose and/or Choice and/or Adverse Reactions and/or Deficiency of; Cancer (Including but Not Limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Rare Diseases and/or Orphan Diseases and/or Metabolic Diseases and/or Syndromes; Heart Disease (Including but Not Limited to Coronary Artery Disease (CAD) and/or Myocardial Infarction); Bleeding Diathesis and/or Coagulation Disorders and/or Hemophilia; Thrombophilia and/or Thromboembolic Disease; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Infectious Disease Susceptibility (Including but Not Limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections); and Age (Including but not Limited to Age Range and/or Approximate or Exact Age).

The Research & Clinical Trial Panel can be utilized to rescue pharmaceutical, biotechnology or medical device clinical trials that are in-danger of failing FDA or EU approval or to resurrect pharmaceutical, biotechnology, or medical device research or clinical trials that had failed previously. This rescue or resurrection can be accomplished by applying information from genetic testing or genetic analysis or both to research or clinical trials in order to evaluate and correlate medication safety-profiles, effectiveness, response, dosing or adverse drug events with one or more specific genetic variants. In some cases, genetic variants may be identified with a negative result such as for example a serious adverse reaction, morbidity, mortality, lack of efficacy, or decreased efficacy. In other cases, genetic variants may be identified with a positive result such as for example efficacy, increased efficacy, or increased subject health. The Research & Clinical Trial Panel can also be utilized to improve pharmaceutical, biotechnology and medical device research efforts and clinical trials by assisting in evaluating safety-profiles, effectiveness, response, dosing or adverse drug events based on the additional data conveyed through genetic testing or genetic analysis or both. This genetic testing and/or genetic analysis is applicable to all phases of research and clinical trials, including animal testing (such as mice, rats, guinea pigs, dogs, cats, pigs, apes, chimpanzees and any other animals) and human trials. Association may be found between a single genetic variant's allele or genotype and a specific phenotype, such as a specific adverse drug event, or an association may be found between multiple genetic variants (in one or more genes and/or loci) and their alleles or genotypes and the phenotype of interest. This panel examines all known pharmacogenomic genetic variants, along with genetic variants associated with specific phenotypes such as clinically relevant phenotypes including but not limited to cardiac arrhythmias, heart disease, universal identifier, cancer, and rare diseases and more and is therefore a more thorough examination of a person's genome as opposed to nothing at all, or as opposed to genetic testing and/or genetic analysis that only takes into account pharmacogenomic-related genes or genetic variants. In other embodiments, a larger number of variants than provided in Research and Clinical Trial Panel such as all the variants in the PMD, or a subset therein may be correlated to clinical trial outcomes. In other embodiments, all variants known to be associated with the particular adverse event that occurred frequently during the clinical trial may be tested and analyzed.

Individuals who are about to undergo surgery or who require anesthesia, or their caretakers, physicians or surgeons, or other medical personnel, may use the Surgery & Anesthesiology Panel and/or the Transplant Panel. Individuals, their physicians, or other medical personnel, may also use an emergency panel, see e.g., the Emergency Panel for emergency situations. An emergency panel may be used to inform or determine a course of treatment of an individual who is treated in the field (ie non-hospital environment), such as by emergency medical technicians, emergency medicine physicians, medics in the armed forces, Red Cross medical personnel, first responders, police, firefighters, etc., or who arrives at a medical center, hospital or clinic through the emergency department, or who arrives to a hospital, clinic or medical center during an emergency, in critical or unstable condition, unconscious, delirious, non-responsive, combative, intoxicated (due to use of alcohol, drugs, etc.), disoriented or cognitively impaired. For example, the Emergency Panel may be used to screen an unconscious or otherwise impaired individual for drug sensitivities, allergies, drug metabolism rates, adverse drug reactions and the like. An individual's pharmacogenomic profile for emergency situations thus can be generated. An individual may select the entire panel, or a subset, of conditions in which their risk or predisposition to the condition is determined. For example, phenotypes, such as at least 1, 2, 3, 4, 5, 6, 7, 8, or 9 of the following phenotypes: Blood Group; Drug Metabolism and/or Choice and/or Sensitivity and/or Adverse Reactions and/or Dosing (Including Pharmacogenomic Analysis for all Pharmaceuticals); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Hypertrophic Cardiomyopathy; Universal Identifier/Identity Testing; Thrombophilia and/or Thromboembolic Disease; Bleeding Diathesis and/or Coagulation Disorders and/or Hemophilia; Susceptibility to Bacteremia and/or Sepsis and/or Severe Sepsis and/or Septic Shock and/or Systemic Inflammatory Response Syndrome; or Wound Dehiscence. An Emergency Panel can determine the risk or predisposition of a subset of the aforementioned phenotypes, such as at least 1, 2, 3, 4, or 5 of the following phenotypes: Blood Group; Drug Metabolism and/or Choice and/or Sensitivity and/or Adverse Reactions and/or Dosing (Including Pharmacogenomic Analysis for all Pharmaceuticals); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Hypertrophic Cardiomyopathy; or Universal Identifier/Identity Testing.

Individuals can also select the Medical Procedure & Interventional Radiology Panel, which can be used to determine the risk or predisposition of an individual for phenotypes, such as at least 1, 2, 3, 4, 5, or 6 of the following phenotypes: Thrombophilia and/or Thromboembolic Disease; Bleeding Diathesis and/or Coagulation Disorders and/or Hemophilia; Allergic Reactions (including but not limited to Food Allergies and/or Environmental Allergies and/or Drug Allergies); Seizures and/or Epilepsy; Latex Allergy; or Medication Metabolism (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea). A Medical Procedure & Interventional Radiology Panel can determine the risk or predisposition of a subset of the aforementioned phenotypes, such as at least 1, 2, or 3 of the following phenotypes: Thrombophilia and/or Thromboembolic Disease; Bleeding Diathesis and/or Coagulation Disorders and/or Hemophilia; or Allergic Reactions (including but not limited to Food Allergies and/or Environmental Allergies and/or Drug Allergies).

The Pharmacology and Alternative Medicine Panel, may also be used to generate a pharmacogenomic profile. For example, all of the phenotypes, or a subset, such as at least 1 or 2 of the following phenotypes: Drug Metabolism and/or Choice and/or Sensitivity and/or Adverse Reactions and/or Dosing (Including Pharmacogenomic Analysis for all Pharmaceuticals); Vitamin and/or Mineral and/or Element and/or Herbal and/or Nutritional Supplement Metabolism and/or Sensitivity and/or Dose and/or Choice and/or Adverse Reactions and/or Deficiency Thereof; or Taste Perception. A Pharmacology & Alternative Medication Panel can determine the risk or predisposition of a subset of the aforementioned phenotypes, such as at least 1, 2, or 3 of the following phenotypes: Drug Metabolism and/or Choice and/or Sensitivity and/or Adverse Reactions and/or Dosing (Including Pharmacogenomic Analysis for all Pharmaceuticals); Vitamin and/or Mineral and/or Element and/or Herbal and/or Nutritional Supplement Metabolism and/or Sensitivity and/or Dose and/or Choice and/or Adverse Reactions and/or Deficiency Thereof; or Taste Perception. Individuals with or without a current or prior diagnosis of an adverse reaction to a medication or alternative treatment may also be interested in other genetic links to phenotypes, and their risk or predisposition to those phenotypes, that are related to medications and alternative treatments, and thus may also be interested in the Pharmacology and Alternative Medicine Panel, for example.

A pharmacogenomic profile can be used to aid physicians and other providers of drugs, treatments, or alternative therapies to identify appropriate medications, drugs, procedures or treatment and to approximate appropriate dosage(s). Similarly, the Allergy and Atopy Panel, or subset thereof, may help healthcare providers, medical personnel or alternative medicine providers (such as an acupuncturist or herbologist) to determine the cause of an individual's allergy, or atopy as well as what materials, medical products, or devices can or should not come in contact with an individual receiving care, such as a substance that may cause an adverse reaction. For example, an individual have determined/analyzed his or her risk or predisposition for phenotypes such as all or at least 1, 2, 3, 4, 5, 6 or 7 of the following phenotypes: Asthma Triggers (including but not limited to Asthma Exacerbations due to Exposure to Dust, Endotoxins, and/or Cockroaches); Allergies and/or Atopy (including but not limited to Food Allergies and/or Environmental Allergies and/or Contact Allergies and/or Rashes and/or Eczema); Atopic Dermatitis; Latex Allergy; Asthma; Response to and/or Effectiveness and/or Dosing and/or Choice and/or Adverse Reactions of Medications used to Treat Asthma including but not Limited to Beta-Agonists and/or Corticosteroids and/or Bronchodilators; Rhinitis and/or Rhinoconjunctivitis. An Allergy and Atopy Panel can determine the risk or predisposition of a subset of the aforementioned phenotypes, such as at least 1, 2, 3, or 4 of the following phenotypes: Asthma Triggers (including but not limited to Asthma Exacerbations due to Exposure to Dust, Endotoxins, and/or Cockroaches); Allergies and/or Atopy (including but not limited to Food Allergies and/or Environmental Allergies and/or Contact Allergies and/or Rashes and/or Eczema); Atopic Dermatitis; or Latex Allergy.

For example, latex-free gloves may be used when treating an individual with a risk of developing a latex allergy as determined by an allergy, asthma and atopy panel. (Brown. Anesthesiology 102(3): 496-502 (2005)) An individual found to be at higher risk for asthma and reflex testing then shows them to be at higher risk of asthma due to cockroach exposure can be advised on this risk and may be able to take necessary measures to eradicate cockroaches from their domicile. In one embodiment, the Allergy and Atopy panel is provided to test an allergy to latex, such as any products containing latex including but not limited to latex gloves, other latex medical devices, or latex condoms. Risks for all of the phenotypes, such as conditions, in the panel, or a subset of the phenotypes, such as conditions, may be determined instead.

For example, the Emergency Panel may be used to generate an individual's pharmacogenomic profile and to determine his or her blood type and/or other aspects of his or her identity. In other situations, the Emergency Panel may be used simply to determine an aspect of the individual's identity. For example, when a hospital admits a patient, either living or decreased, with an unknown identity, such as a “John Doe” or “Jane Doe”, the Universal Identifier enables the healthcare provider to run this person's genetic Universal Identification against any database (their own or other databases, such as local, state, federal, or international missing persons databases or other police/governmental databases) that may contain this information. The healthcare provider may also store the Universal Identification so that identification can be made at a later date or if needed for healthcare, government, or forensic police work now or in the future. In still other examples, panels such as the Emergency Panel, the Pharmacology and Alternative Medicine Panel, and/or the Allergy, Asthma and Atopy Panel can be applied in non-emergency settings, for example, to guide the treatment of an individual receiving critical care, routine medical care, in a disaster situation or area, surgery or complementary or alternative care.

One or more panels described herein, or subsets thereof, may be used before any type of medical procedure. For example, the Surgery and Anesthesiology Panel can be used to determine the risk or predisposition of all of the phenotypes, or a subset, such as at least 1, 2, 3, 4, 5, 6, 7, 8, 9, 10, 11, 12, or 13 of the following phenotypes: Blood Group; Malignant Hyperthermia; Postanesthetic Apnea; Analgesic Effectiveness of Opiates; Wound Dehiscence; Nitrous Oxide Sensitivity; Thrombophilia and/or Thromboembolic Disease; Bleeding Diathesis and/or Coagulation Disorders and/or Hemophilia; Wolff-Parkinson-White Syndrome; Arrhythmogenic Right Ventricular Cardiomyopathy; Anesthesia Requirements for Proper Sedation; Level of Post-operative Pain; or Latex Allergy can be determined. A Surgery & Anesthesiology Panel can determine the risk or predisposition of a subset of the aforementioned phenotypes, such as at least 1, 2, 3, 4, or 5 of the following phenotypes: Blood Group; Malignant Hyperthermia; Postanesthetic Apnea; Analgesic Effectiveness of Opiates; or Wound Dehiscence. The panel may be used to test an individual needing care in an emergency or non-emergency setting.

In some cases, the Transplant Panel (or subset thereof) may be used to test either or both the donor (whether living or decreased) and/or the recipient of the transplant or an individual requiring emergency or immediate treatment.

In another example, an individual undergoing a routine surgery may be tested using one or more (or a subset of one or more): Surgery & Anesthesiology Panel, or a subset thereof); the Transplant Panel (or a subset), such as at least 1, 2, 3, 4, 5, 6, 7, 8, 9, 10, or 11 of the following phenotypes: Blood Group; Human Leukocyte Antigen Typing; Malignant Hyperthermia; Postanesthetic Apnea; Prognosis following Transplantation; Wound Dehiscence; Graft Versus Host Disease; Thrombophilia and/or Thromboembolic Disease; Bleeding Diathesis and/or Coagulation Disorders and/or Hemophilia; Anesthesia Requirements for Proper Sedation; or Level of Post-operative Pain. A Transplant Panel can determine the risk or predisposition of a subset of the aforementioned phenotypes, such as at least 1, 2, 3, 4, 5, or 6 of the following phenotypes: Blood Group; Human Leukocyte Antigen Typing; Malignant Hyperthermia; Postanesthetic Apnea; Prognosis following Transplantation; or Wound Dehiscence.

Specific transplant panels may be used depending upon the organ, organ system, or tissue to be transplanted. For example, a kidney transplant panel (e.g., the Kidney Transplant Panel) may be used to assess an individual with anemia, a glomerular filtration rate (GFR) less than 25% of normal or a GFR that is decreasing over time, oliouria, anuria, pericarditis, and/or neuropathy. Similarly, a patient undergoing, or planning to undergo, dialysis, or with a personal or family medical history of renal insufficiency, renal failure, end-stage renal disease, kidney transplant, diabetes mellitus, hypertension, malignant hypertension, syndrome-related kidney disease, glomerulonephritis, polycystic kidney disease, lupus, or Goodpasture's syndrome may also benefit from a kidney transplant panel. The Kidney Transplant Panel, which can be used to determine the risk or predisposition of an individual for phenotypes, such as at least 1, 2, 3, 4, 5, 6, 7, or 8 of the following phenotypes: Prognosis and/or Survival following Kidney Transplant (including but not limited to Graft Survival in Kidney Transplant Recipients, Survival Advantage in Kidney Transplant Recipients, Sensitivity and/or Adverse Drug Reactions and/or Dosing of Medications given to Kidney Transplant Recipients, and/or Risk of CMV Infection After Kidney Transplantation); Human Leukocyte Antigen Typing; Blood Group; Malignant Hyperthermia; Postanesthetic Apnea; Thrombophilia and/or Thromboembolic Disease; Bleeding Diathesis and/or Coagulation Disorders and/or Hemophilia; or Wound Dehiscence. A Kidney Transplant Panel can determine the risk or predisposition of a subset of the aforementioned phenotypes, such as at least 1, 2, 3, 4, 5, or 6 of the following phenotypes: Prognosis and/or Survival following Kidney Transplant (including but not limited to Graft Survival in Kidney Transplant Recipients, Survival Advantage in Kidney Transplant Recipients, Sensitivity and/or Adverse Drug Reactions and/or Dosing of Medications given to Kidney Transplant Recipients, and/or Risk of CMV Infection After Kidney Transplantation); Human Leukocyte Antigen Typing; Blood Group; Malignant Hyperthermia; Postanesthetic Apnea; or Thrombophilia and/or Thromboembolic Disease.

A lung transplant panel may be of benefit to an individual who is a current or former tobacco smoker; an individual who is experiencing, or has experienced, symptoms such as dyspnea, fatigue, chest pain, clubbing; an individual with a Pulmonary Function Test (PFT) result of Forced Expiratory Volume in 1 Second (FEV1) less than between about 75% and about 25% of the normal value or the predicted value, a FEV1 that is decreasing over time, PFTs indicative of severe lung damage or respiratory failure, or with lab tests indicative of or symptoms associated with respiratory damage such as hypoxia, hypoxemia, and/or hypercapnia. A lung transplant panel may also be of use to an individual with a family or personal medical history of Respiratory or Pulmonary failure, end-stage lung disease, lung transplant, chronic obstructive pulmonary disease, idiopathic pulmonary fibrosis; Cystic fibrosis; idiopathic pulmonary hypertension, alpha 1-antitrypsin deficiency, bronchiectasis, or sarcoidosis.

For example, individuals may select the Lung Transplant Panel, which can be used to determine the risk or predisposition of an individual for phenotypes, such as at least 1, 2, 3, 4, 5, 6, 7, or 8 of the following phenotypes: Prognosis and/or Survival following Lung Transplant (including but not limited to Graft Survival in Lung Transplant Recipients and/or Survival Advantage in Lung Transplant Recipients); Blood Group; Thrombophilia and/or Thromboembolic Disease; Human Leukocyte Antigen Typing; Bleeding Diathesis and/or Coagulation Disorders and/or Hemophilia; Wound Dehiscence; Malignant Hyperthermia; or Postanesthetic Apnea. A Lung Transplant Panel can determine the risk or predisposition of a subset of the aforementioned phenotypes, such as at least 1, 2, 3, or 4 of the following phenotypes: Prognosis and/or Survival following Lung Transplant (including but not limited to Graft Survival in Lung Transplant Recipients and/or Survival Advantage in Lung Transplant Recipients); Blood Group; Thrombophilia and/or Thromboembolic Disease; or Human Leukocyte Antigen Typing. Individuals found at risk for one or more of the diseases mentioned herein that are indications for a possible lung transplant, or individuals diagnosed with any of the diseases mentioned herein that are indications for a possible lung transplant, may automatically reflex to test for and/or analyze part of, or the entire, Lung Transplant Panel. For example, if an individual is found to carry or likely have pulmonary arterial hypertension after genetic testing and/or analysis in any panel or is diagnosed with pulmonary arterial hypertension, then reflex testing and/or analysis may occur for either part of, or the entire, Lung Transplant Panel.

In another example, a Liver Transplant Panel may be used to assess an individual with a history of alcohol abuse or dependence or test results indicating a high or elevated ammonia level, high or elevated bilirubin level, low albumin level, high or elevated international normalized ratio (INR), or hypoglycemia. A liver transplant panel may also be useful to an individual with symptoms of jaundice, pruritus, or encephalopathy or who has previously overdosed on a hepatoxic substance, e.g. Acetaminophen (Paracetamol, Tylenol). A liver panel may also be used to test an individual with a family or personal medical history of liver failure, liver transplant, viral hepatitis, cirrhosis, or alpha 1-antitrypsin deficiency.

For example, individuals may select the Liver Transplant Panel, which can be used to determine the risk or predisposition of an individual for phenotypes, such as at least 1, 2, 3, 4, 5, 6, 7, or 8 of the following phenotypes: Prognosis and/or Survival following Liver Transplant (including but not limited to Graft Survival in Liver Transplant Recipients, Survival Advantage in Liver Transplant Recipients, Adverse Drug Reactions with Medications given to Liver Transplant Recipients, and/or Risk of Hepatitis Recurrence after Liver Transplantation); Blood Group; Human Leukocyte Antigen Typing; Thrombophilia and/or Thromboembolic Disease; Bleeding Diathesis and/or Coagulation Disorders and/or Hemophilia; Wound Dehiscence; Malignant Hyperthermia; or Postanesthetic Apnea.

A Liver Transplant Panel can determine the risk or predisposition of a subset of the aforementioned phenotypes, such as at least 1, 2, 3, or 4 of the following phenotypes: Prognosis and/or Survival following Liver Transplant (including but not limited to Graft Survival in Liver Transplant Recipients, Survival Advantage in Liver Transplant Recipients, Adverse Drug Reactions with Medications given to Liver Transplant Recipients, and/or Risk of Hepatitis Recurrence after Liver Transplantation); Blood Group; Human Leukocyte Antigen Typing; or Thrombophilia and/or Thromboembolic Disease. Individuals found at risk for one or more of the diseases mentioned herein that are indications for a possible liver transplant, or individuals diagnosed with any of the diseases mentioned herein that are indications for a possible stem cell transplant, may automatically reflex to test for and/or analyze part of, or the entire, Liver Transplant Panel. For example, if an individual is found to carry or likely have alpha 1-antitrypsin deficiency after genetic testing and/or analysis in any panel or is diagnosed with alpha 1-antitrypsin deficiency, then reflex testing and/or analysis may occur for either part of, or the entire, Liver Transplant Panel.

In yet another example, a stem cell transplant panel may be used to test an individual suffering from a wide range of symptoms, diseases or disorders. A stem cell transplant panel may be especially useful in cases where an individual is contemplating undergoing, or preparing to undergo, stem cell therapy. A stem cell transplant panel may be used if a stem cell transplant indicated as treatment for the disease or an individual who will not benefit from prolonged treatment with, or who is already resistant to, medication, chemotherapy, radiation, or total body radiation may use, a stem cell transplant panel; an individual with specific symptoms (e.g., fatigue, weakness) or with a family history of stem cell transplant may use a stem cell panel. Similarly, a stem cell transplant panel may be used to test an individual with a family or personal medical history of neurodegenerative disease (such as Parkinson's Disease or Alzheimer's Disease or Amyotrophic Lateral Sclerosis or Huntington's Disease), multiple myeloma, leukemia, lymphoma, HIV Infection and/or AIDS, Severe combined immunodeficiency, congenital neutropenia, aplastic anemia, sickle-cell disease, Myelodysplastic syndrome, neuroblastoma, Ewing's sarcoma, desmoplastic tumor, Hodgkin's disease, liver disease (e.g., hepatitis, cirrhosis), cardiovascular disease, pancreatic disease (e.g., pancreatic cancer, diabetes) or other disease or disorder that may be treatable with a stem cell transplant. The stem cells to be transplanted may any type or form of stem cell, such as totipotent cells (e.g., cells produced from the fusion of an egg and sperm cell as well as cells produced by the first few divisions of the fertilized egg), pluripotent cells (e.g., embryonic stem cells, induced pluripotent cells), multipotent cells (e.g., mesenchymal stem cells, hematopoietic stem cells, adipose-derived stem cells, endothelial stem cells etc.), or unipotent cells (e.g., muscle stem cells). Individuals found at risk for one or more of the diseases mentioned herein that are indications for a possible stem cell transplant, or individuals diagnosed with any of the diseases mentioned herein that are indications for a possible stem cell transplant, may automatically reflex to test for and/or analyze part of, or the entire, Stem Cell Transplant Panel.

Thus, individuals may select the Stem Cell Transplant Panel, which can be used to determine the risk or predisposition of an individual for phenotypes, such as at least 1, 2, 3, 4, or 5 of the following phenotypes: Prognosis and/or Survival following Stem Cell Transplant (including but not limited to Stem Cell Survival in Transplant Recipients, Survival Advantage in Stem Cell Transplant Recipients, and/or Sensitivity to or Adverse Drug Reactions with Medications given to Stem Cell Transplant Recipients); Graft Versus Host Disease; Human Leukocyte Antigen Typing; Blood Group; or Susceptibility to Bacteremia and/or Sepsis and/or Severe Sepsis and/or Septic Shock and/or Systemic Inflammatory Response Syndrome. A Stem Cell Transplant Panel can determine the risk or predisposition of a subset of the aforementioned phenotypes, such as at least 1, 2, or 3 of the following phenotypes: Prognosis and/or Survival following Stem Cell Transplant (including but not limited to Stem Cell Survival in Transplant Recipients, Survival Advantage in Stem Cell Transplant Recipients, and/or Sensitivity to or Adverse Drug Reactions with Medications given to Stem Cell Transplant Recipients); Graft Versus Host Disease; or Human Leukocyte Antigen Typing.

Risks or carrier status or both, for phenotypes for all of these panels, or a subset of phenotypes, such as conditions, therein, may be tested. In some cases, an emergency panel may be used to test an individual for a set of two or more risks (or predisposition), or carrier status, for example, such set may include one of the following sets of risks or dispositions: predisposition to react to medications (including metabolism of drugs, allergies, adverse reactions, sensitivities, dosing) and identification of a specific blood group; predisposition to adversely react to medications and likelihood of specific characteristics of identity; predisposition to react to anesthesia and risk of postanesthetic apnea; predisposition for thrombosis or thromboembolic disease and identification of any bleeding diathesis, coagulation disorders, or hemophilia; malignant hyperthermia and wound dehiscence. In some cases, a surgery and anesthesiology panel may be used to test an individual for a set of two or more risks (or predisposition) or carrier status, for example, such set may include one of the following sets of risks: predisposition for certain level of anesthesia to achieve sedation and predisposition for analgesic effect from opiates; risk of malignant hyperthermia and risk of wound dehiscence; predisposition for sensitivity to nitrous oxide and predisposition for certain level of anesthesia to achieve sedation; risk of malignant hyperthermia and predisposition to react to opiates; risk of malignant hyperthermia and predisposition for a specific blood group.

Individuals, such as those who have one or more of the following: a personal history of blood clot(s), myocardial infarction(s), transient ischemic attack(s), stroke(s), or pulmonary embolism(s), a family history of blood clots, myocardial infarction(s), transient ischemic attack(s), stroke(s), or pulmonary embolism(s), who have to or may have to remain stationary or immobile for extended periods of time (such as due to being in a cast, wheelchair, coma, having recent surgery, taking long airplane flight(s), taking long auto trip(s), training for or going on space mission(s), serving in an armored tank division of an armed forces, etc.), are patients in a critical care or intensive care unit, have a condition or conditions that may increase their risk of blood clots, such as having an acute or chronic infection, sepsis, systemic inflammatory response syndrome (SIRS), atrial fibrillation, polycythemia vera, or any type of cancer, may take or are taking certain medications that may increase their risk of thrombosis, such as oral contraceptive pills (OCPs), hormone replacement therapy (HRT), letrozole, or estrogen, or who are cigarette smokers, live with cigarette smokers, or are exposed to cigarette smoke, may also select the Blood Flow, Thrombosis and Thromboembolism Panel, which can be used to determine the risk or predisposition of an individual for phenotypes, such as at least 1, 2, 3, 4, 5, 6, or 7 of the following phenotypes: Thrombophilia and/or Thromboembolic Disease; Warfarin Metabolism and/or Sensitivity and/or Adverse Reaction and/or Dosing; Effectiveness and/or Sensitivity and/or Adverse Reactions and/or Dose and/or Choice of Anti-hyperlipidemic and/or Anti-atherosclerotic and/or Anti-Restenosis Medications and/or NSAIDs (including but not limited to Acetylsalicylic Acid); Homocysteine Level; Stroke (CVA); Myocardial Infarction; or Coronary Artery Disease (CAD). A Blood Flow, Thrombosis and Thromboembolism Panel can determine the risk or predisposition of a subset of the aforementioned phenotypes, such as at least 1, 2, 3, or 4 of the following phenotypes: Thrombophilia and/or Thromboembolic Disease; Warfarin Metabolism and/or Sensitivity and/or Adverse Reaction and/or Dosing; Effectiveness and/or Sensitivity and/or Adverse Reactions and/or Dose and/or Choice of Anti-hyperlipidemic and/or Anti-atherosclerotic and/or Anti-Restenosis Medications and/or NSAIDs (including but not limited to Acetylsalicylic Acid); or Homocysteine Level.

Individuals may also select the Blood Panel, which can be used to determine the risk or predisposition of an individual for phenotypes, such as at least 1, 2, 3, 4, 5, 6, 7, or 8 of the following phenotypes: Blood Group and Hemoglobin Variants; Anemia and/or Abnormalities of the Blood; Thrombophilia and/or Thromboembolic Disease; Bleeding Diathesis and/or Coagulation Disorders and/or Hemophilia; Thalassemia; Sickle Cell Anemia and/or Sickle Cell Trait; Malaria Susceptibility; or Universal Identifier/Identity Testing. A Blood Panel can determine the risk or predisposition of a subset of the aforementioned phenotypes, such as at least 1, 2, 3, or 4 of the following phenotypes: Blood Group and Hemoglobin Variants; Anemia and/or Abnormalities of the Blood; Thrombophilia and/or Thromboembolic Disease; or Bleeding Diathesis and/or Coagulation Disorders and/or Hemophilia.

Like other panels described herein, the Cardiovascular Panels Alpha and Beta has many possible uses and applications: In some cases, the Cardiovascular Panels (or subset thereof) may be used to test an individual with one or more of the following conditions, symptoms or test results: abnormal lipid level(s) (including but not limited to abnormal cholesterol level), abnormal electrocardiogram, abnormal echocardiogram, coronary artery stenosis, pain or discomfort in the upper body (e.g., chest, back, arm, neck, throat, or jaw), dyspnea, and/or heart palpitation. The Cardiovascular Panels (or subset thereof) may be used to test a current or former tobacco smoker and/or an individual whose past or current medical history includes one or more of the following: diabetes mellitus, obesity, above-normal weight, angina, myocardial infarction, heart arrhythmia, or other heart disease. The results from such tests may contribute to a plan of treatment for the individual or a plan to change his or her lifestyle. For example, an individual with a history of chest pain or discomfort may be tested for his or her risk or predisposition to coronary artery disease, myocardial infarction or other cardiovascular disease or condition. Such individual may also be tested for the likelihood a food or beverage will enhance his or her risk of a cardiovascular disease or condition. Depending on the results, a nutritional diet or program tailored to such individual's risk for cardiovascular disease may be designed. Individuals with a particular family history of cardiovascular disease or conditions may also be tested using the Cardiovascular and Cardiology Panels or subset thereof. For example, an individual with a family history of heart disease (e.g., atherosclerosis, myocardial infarction, sudden death, cardiomyopathy, angina, high cholesterol level, high lipid level, heart failure, hypertensive heart disease) may also be tested using the Cardiovascular Panels, or subset thereof.

For example, individuals may select the Cardiovascular Panel Alpha, which can be used to determine the risk or predisposition of an individual for phenotypes, such as at least 1, 2, 3, 4, 5, 6, 7, 8, 9, 10, 11, 12, 13, 14, or 15 of the following phenotypes: Heart Disease (including but not limited to Coronary Artery Disease (CAD) and/or Myocardial Infarction); Hypertension and/or Blood Pressure Level; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Thrombophilia and/or Thromboembolic Disease; or Cardiomyopathy. A Cardiovascular Panel Alpha can determine the risk or predisposition of a subset of the aforementioned phenotypes, such as at least 1, 2, 3, 4, 5, 6, 7, or 8 of the following phenotypes: Heart Disease (including but not limited to Coronary Artery Disease (CAD) and/or Myocardial Infarction); Hypertension and/or Blood Pressure Level; Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Thrombophilia and/or Thromboembolic Disease; Cardiomyopathy; Heart Failure; Peripheral Arterial Disease; or Structural Heart Defect.

The Cardiovascular Panel Beta can be used to determine the risk or predisposition of an individual such as for all or at least 1, 2, 3, 4, 5, 6, 7, 8, 9, 10, 11, 12, 13, 14, 15, or 16 of the following phenotypes: Coronary Artery Disease (CAD); Myocardial Infarction; Thrombophilia and/or Thromboembolic Disease; Wolff-Parkinson-White Syndrome; Atrial Fibrillation; Hypertrophic Cardiomyopathy; Arrhythmogenic Right Ventricular Cardiomyopathy; Dyslipidemia (Including Total Cholesterol and/or LDL Cholesterol and/or HDL Cholesterol and/or Triglycerides and/or Chylomicrons); Hypertension and/or Blood Pressure Level; Heart Failure; Dilated Cardiomyopathy; Coronary Artery Spasm; Aortic and/or Arterial Aneurysm and/or Dissection; Effects of Specific Foods (including but not limited to Fruits and/or Vegetables) and/or Beverages (including but not limited to Alcohol and/or Caffeine) Consumption on Heart Health and/or Risk of Atherosclerosis and/or Risk of Myocardial Infarction; Long QT Syndrome; or Brugada Syndrome. A Cardiovascular Panel Beta can determine the risk or predisposition of a subset of the aforementioned phenotypes, such as at least 1, 2, 3, 4, 5, 6, 7, 8, or 9 of the following phenotypes: Coronary Artery Disease (CAD); Myocardial Infarction; Thrombophilia and/or Thromboembolic Disease; Wolff-Parkinson-White Syndrome; Atrial Fibrillation; Hypertrophic Cardiomyopathy; Arrhythmogenic Right Ventricular Cardiomyopathy; Dyslipidemia (Including Total Cholesterol and/or LDL Cholesterol and/or HDL Cholesterol and/or Triglycerides and/or Chylomicrons); or Hypertension and/or Blood Pressure Level.

Individuals interested in the Cardiovascular Panels may also be interested in the Heart Failure Panel, Coronary Artery Disease Panel, Myocardial Infarction Panel, Lipid Level Panel, Blood Pressure Panel, Stroke Panel or subsets there of, as well as the Heartbeat/Arrhythmia Panel or Dyslipidemia Panel. Alternatively, individuals interested in any of those panels, or subsets thereof, may be interested in one of the aforementioned panels or subsets thereof.

For example, an individual interested in the Heart Failure Panel, such as someone with abnormal brain natriuretic peptide (BNP) levels or an abnormal echocardiogram and/or suffering from dyspnea; has difficulty breathing; has swollen lower extremities; and/or has upper extremity pain may be interested in phenotypes such as or at least 1, 2, 3, 4, or 5 of the following phenotypes: Heart Failure; Effectiveness and/or Therapeutic Response and/or Pharmacogenomics of and/or Choice of Interventions with Heart Failure (including but not limited to Beta Blocker Therapy); Survival and/or Prognosis with Congestive Heart Failure; Heart Disease (including but not limited to Coronary Artery Disease (CAD) and/or Myocardial Infarction); or Thrombophilia and/or Thromboembolic Disease. A Heart Failure Panel can determine the risk or predisposition of a subset of the aforementioned phenotypes, such as at least 1, 2, or 3 of the following phenotypes: Heart Failure; Effectiveness and/or Therapeutic Response and/or Pharmacogenomics of and/or Choice of Interventions with Heart Failure (including but not limited to Beta Blocker Therapy); or Survival and/or Prognosis with Congestive Heart Failure. Individuals with or without a current or prior diagnosis of heart failure may also be interested in other genetic links to phenotypes, and their risk or predisposition to those phenotypes, that are related to heart failure, and thus may also be interested in the Heart Failure Panel, for example.

Individuals may also select the Heartbeat/Arrhythmia Panel, which can be used to determine the risk or predisposition of an individual for phenotypes, such as at least 1, 2, 3, 4, 5, 6, 7, 8, 9, 10, 11, 12, or 13 of the following phenotypes: Atrial Fibrillation; Long QT Syndrome; Drug-induced Long QT Syndrome; Drug-induced Torsade de Pointes; Ventricular Fibrillation; Ventricular Tachycardia; Arrhythmogenic Right Ventricular Cardiomyopathy; Wolff-Parkinson-White Syndrome; Brugada Syndrome; Heart Block; Effectiveness and/or Choice and/or Pharmacogenomics of Antiarrhythmogenic Medication; Digoxin Metabolism and/or Toxicity; or Thrombophilia and/or Thromboembolic Disease. A Heartbeat/Arrhythmia Panel can determine the risk or predisposition of a subset of the aforementioned phenotypes, such as at least 1, 2; 3, 4, 5, 6, 7, 8, 9, 10, 11, or 12 of the following phenotypes: Atrial Fibrillation; Long QT Syndrome; Drug-induced Long QT Syndrome; Drug-induced Torsade de Pointes; Ventricular Fibrillation; Ventricular Tachycardia; Arrhythmogenic Right Ventricular Cardiomyopathy; Wolff-Parkinson-White Syndrome; Brugada Syndrome; Heart Block; Effectiveness and/or Choice and/or Pharmacogenomics of Antiarrhythmogenic Medication; or Digoxin Metabolism and/or Toxicity. Individuals with or without a current or prior diagnosis of a cardiac arrhythmia may also be interested in other genetic links to phenotypes, and their risk or predisposition to those phenotypes, that are related to a cardiac arrhythmia, and thus may also be interested in the Heartbeat/Arrhythmia Panel, for example.

Individuals may select the Dyslipidemia Panel, which can be used to determine the risk (as previously stated, the term ‘risk’ may refer to one or more of the following: increased or decreased risk of multifactorial phenotype(s) or carrier status of monogenic or polygenic phenotypes, including carrier, non-carrier, affected, or likely affected) or predisposition of an individual for phenotypes, such as at least 1, 2, 3, 4, 5, 6, 7, or 8 of the following phenotypes: Dyslipidemia; Dosage Required of Statin to Reduce Risk of Death or Major Cardiovascular Events; Statin-Induced Rhabdomyolysis and/or Myopathy; Change in Body Fat and/or Lipid Levels with Specific Diets and/or Exercise; Risk of Acute Coronary Syndrome with Preexisting Coronary Artery Disease; Effectiveness of and/or Sensitivity to and/or Intolerance to and/or Resistance to Anti-hyperlipidemic and/or Anti-atherosclerotic and/or Anti-Restenosis Medication; Severity of Coronary Atherosclerosis with Coronary Artery Disease; Degree of Cognitive Decline after Coronary Artery Bypass Graft Surgery; or Restenosis Following Coronary Angioplasty. A Dyslipidemia Panel can determine the risk or predisposition of a subset of the aforementioned phenotypes, such as at least 1, 2, 3, or 4 of the following phenotypes: Dyslipidemia; Dosage Required of Statin to Reduce Risk of Death or Major Cardiovascular Events; Statin-Induced Rhabdomyolysis and/or Myopathy; or Change in Body Fat and/or Lipid Levels with Specific Diets and/or Exercise. Individuals with or without a current or prior diagnosis of dyslipidemia or hyperlipidemia, including elevated cholesterol levels, may also be interested in other genetic links to phenotypes, and their risk or predisposition to those phenotypes, that are related to dyslipidemia or hyperlipidemia, and thus may also be interested in the Dyslipidemia Panel, for example.

An individual may choose to be tested with the Coronary Artery Disease Panel and/or Myocardial Infarction Panel as well, or after results from being tested with the Heart Failure Panel. The individual selecting the Coronary Artery Disease Panel may have abnormal lipid levels, abnormal cardiac stress levels, an abnormal echocardiogram, or an abnormal electrocardiogram. The individual may be greater than approximately 25, 30, 35, or 40 years of age, a current or former tobacco smoker, have Type A behavioral patterns, experience lots of stress, have a diet high in saturated fat, or a combination thereof. The individual may be experiencing upper body pain or discomfort (including but not limited to chest, back, arm, neck, throat and/or jaw), dyspnea, heart palpitation, or a combination thereof. Individuals with or without a current or prior diagnosis of CAD or elevated lipid levels may also be interested in other genetic links to phenotypes, and their risk or predisposition to those phenotypes, that are related to CAD, and thus may also be interested in the CAD Panel, for example.

The individual can select all of the phenotypes, or a subset, such as at least 1, 2, 3, 4, 5, 6, 7, or 8 of the following phenotypes: Coronary Artery Disease (CAD); Effectiveness and/or Sensitivity and/or Adverse Reactions and/or Dose and/or Choice of Anti-hyperlipidemic and/or Anti-atherosclerotic and/or Anti-Restenosis Medications, and/or NSAIDs; Risk of Acute Coronary Syndrome with Preexisting Coronary Artery Disease; Degree of Cognitive Decline after Coronary Artery Bypass Graft Surgery; Restenosis Following Coronary Angioplasty; Statin-Induced Rhabdomyolysis and/or Myopathy; Level of Severity of Coronary Atherosclerosis with CAD; or Association of Specific Food (including but not limited to Fruits and/or Vegetables) and/or Beverage (including but not limited to Alcohol and/or Caffeine) Consumption on Risk of Atherosclerosis and/or Myocardial Infarction; Homocysteine Level. A Coronary Artery Disease Panel can determine the risk or predisposition of a subset of the aforementioned phenotypes, such as at least 1, 2, 3, 4, 5, or 6 of the following phenotypes: Coronary Artery Disease (CAD); Effectiveness and/or Sensitivity and/or Adverse Reactions and/or Dose and/or Choice of Anti-hyperlipidemic and/or Anti-atherosclerotic and/or Anti-Restenosis Medications and/or NSAIDs; Risk of Acute Coronary Syndrome with Preexisting Coronary Artery Disease; Degree of Cognitive Decline after Coronary Artery Bypass Graft Surgery; Restenosis Following Coronary Angioplasty; or Statin-Induced Rhabdomyolysis and/or Myopathy.

An individual selecting the Myocardial Infraction Panel may have elevated homocysteine levels, abnormal lipid levels, abnormal cardiac stress, abnormal echocardiogram, or a blood test indicative of myocardial infarction (such as elevated troponins). The individual may be a current or former tobacco smoker, have Type A behavioral patterns or Type A personality, experience lots of or increased stress, have a diet high in saturated fat, or a combination thereof. The individual may be experiencing upper body pain (including but not limited to chest, back, arm, neck, throat and/or jaw), dyspnea, heart palpitation, or a combination thereof. Individuals with or without a current or prior diagnosis of myocardial infarction or CAD may also be interested in other genetic links to phenotypes, and their risk or predisposition to those phenotypes, that are related to myocardial infarction or CAD, and thus may also be interested in the Myocardial Infarction Panel, for example. The individual may select all of the phenotypes, or a subset, such as at least 1, 2, 3, 4, 5, 6, 7, or 8 of the following phenotypes: Myocardial Infarction; Effectiveness and/or Sensitivity and/or Adverse Reactions and/or Dose and/or Choice of Anti-hyperlipidemic and/or Anti-atherosclerotic and/or Anti-Restenosis Medications and/or NSAIDs; Restenosis Following Coronary Angioplasty; Degree of Cognitive Decline after Coronary Artery Bypass Graft Surgery; Sudden Cardiac Death including Cardiac Arrhythmia and/or Conduction Abnormalities; Stressful Life Events causing Depressive Symptoms and/or Diagnosable Depression and/or Suicidality and/or Anxiety (including but not limited to Mental Vulnerability to Stress and/or Disease); Association of Specific Food (including but not limited to Fruits and/or Vegetables) and/or Beverage (including but not limited to Alcohol and/or Caffeine) Consumption on Risk of Atherosclerosis and/or Myocardial Infarction; or Coronary Artery Disease (CAD). A Myocardial Infarction Panel can determine the risk or predisposition of a subset of the aforementioned phenotypes, such as at least 1, 2, 3, 4, or 5 of the following phenotypes: Myocardial Infarction; Effectiveness and/or Sensitivity and/or Adverse Reactions and/or Dose and/or Choice of Anti-hyperlipidemic and/or Anti-atherosclerotic and/or Anti-Restenosis Medications and/or NSAIDs; Restenosis Following Coronary Angioplasty; Degree of Cognitive Decline after Coronary Artery Bypass Graft Surgery; or Sudden Cardiac Death including Cardiac Arrhythmia and/or Conduction Abnormalities.

An individual selecting the Lipid Level Panel may have abnormal lipid levels and may be greater than approximately 25, 30, 35, or 40 years of age, a current or former tobacco smoker, have Type A behavioral patterns, experience lots of stress, have a diet high in saturated fat, or a combination thereof. The individual may be experiencing upper body pain (including but not limited to chest, back, arm, neck, throat and/or jaw), dyspnea, heart palpitation, or a combination thereof. The individual use the Lipid Level Panel to determine the risk or predisposition of an individual for phenotypes, such as at least 1, 2, 3, 4, 5, or 6 of the following phenotypes: Lipid Levels and/or Dyslipidemia (Including Total Cholesterol and/or LDL Cholesterol and/or HDL Cholesterol and/or Triglycerides and/or Chylomicrons); Anti-hyperlipidemic and/or Anti-atherosclerotic and/or Anti-Restenosis Medication Effectiveness and/or Sensitivity and/or Adverse Reactions and/or Dosing; Change in Body Fat and/or Lipid Levels on Specific Diets and/or with Exercise; Level of Severity of Coronary Atherosclerosis; Coronary Artery Disease (CAD); or Myocardial Infarction. A Lipid Level Panel can determine the risk or predisposition of a subset of the aforementioned phenotypes, such as at least 1, 2, 3, or 4 of the following phenotypes: Lipid Levels and/or Dyslipidemia (Including Total Cholesterol and/or LDL Cholesterol and/or HDL Cholesterol and/or Triglycerides and/or Chylomicrons); Anti-hyperlipidemic and/or Anti-atherosclerotic and/or Anti-Restenosis Medication Effectiveness and/or Sensitivity and/or Adverse Reactions and/or Dosing; Change in Body Fat and/or Lipid Levels on Specific Diets and/or with Exercise; or Level of Severity of Coronary Atherosclerosis in which their risk or predisposition to the phenotype, such as condition, is determined.

An individual selecting the Blood Pressure Panel may have abnormal blood pressure, stress, diet high in salt; diet containing liquorice, or a combination thereof. The individual may be experiencing headaches, tinnitus, fatigue; dizziness; blurred vision, or a mixed of such symptoms. The individual may also have a history, or family history, of hypertension, hypotension, sleep apnea, or combination thereof. The individual may select all or at least 1, 2, 3, 4, or 5 of the following phenotypes: Hypertension and/or Blood Pressure Level; Effectiveness and/or Adverse Reactions and/or Choice and/or Dose of Medications Used to Treat Hypertension (including but not limited to ACE Inhibitors, Angiotensin II Receptor Antagonists, Alpha Blockers, Beta Blockers, Calcium Channel Blockers, Direct Renin Inhibitors, Diuretics, and/or Combination Medications); Association of Specific Diets and/or Consumption of Specific Foods and/or Beverages on Blood Pressure; Carotid Atherosclerosis due to Hypertension; or Kidney Disease due to Hypertension including but not limited to End-Stage Kidney Disease, in which their risk or predisposition to the phenotype, such as condition, is determined.

An individual with an abnormal neurological physical exam, a radiologic exam of the head suggestive and/or indicative of a stroke or transient ischemic attack, an abnormal FAST (Facial weakness, Arm weakness, Speech problems, all Three) test; abnormal homocysteine levels, or a combination thereof may select the Stroke Panel. The individual may be greater than approximately 25, 35, 40, or 45 years of age, a current or former tobacco smoker, and may be suffering from hemiplegia and/or muscle weakness, atrial fibrillation, gait disturbance, numbness, altered smell, taste, hearing and/or vision; ptosis, vertigo, aphasia, dysphasia, or a combination thereof. The individual may also have a history, or family history of transient ischemic attack and/or stroke, atrial fibrillation, hypertension, elevated blood lipid level, diabetes mellitus, carotid stenosis, hypercoagulable state, hemorrhagic state, be on anticoagulation medications, or a combination thereof. The individual may select all of the phenotypes, or a subset, such as at least 1, 2, 3, 4, 5, or 6 of the following phenotypes: Stroke (CVA); Intracranial Aneurysm; Warfarin Metabolism and/or Sensitivity and/or Adverse Reaction and/or Dosing; Antithrombotic Effectiveness of Acetylsalicylic Acid; Thrombophilia and/or Thromboembolic Disease; or Atrial Fibrillation, in which their risk or predisposition to the phenotype, such as condition, is determined.

The Dermatology Panel may be used by individuals concerned about their skin or about hair loss. For example, an individual whose lifestyle involves time in the sun may be tested using the Dermatology Panel in order to determine his or her risk of, or predisposition for, skin cancer. An individual with a current precancerous or cancerous skin lesion, or a history of precancerous or cancerous skin lesions, or other condition (e.g., dermatitis, Stevens-Johnson syndrome, toxic epidermal necrolysis, erythema multiforme, erythema multiforme major or other disorder) or symptom (e.g., abnormal rash or lesion) may also be tested using the Dermatology Panel in order to determine his or her risk for a disease or condition listed in the Dermatology Panel. The Dermatology Panel may also be used for individuals with a family history of skin diseases (e.g., skin cancer, dermatitis, Stevens-Johnson syndrome, toxic epidermal necrolysis, erythema multiforme, erythema multiforme major) or hair loss. An individual may choose all of the phenotypes listed, or a subset, such as at least 1, 2, 3, 4, 5, 6, or 7 of the following phenotypes: Melanoma; Non-melanoma Skin Cancer; Sensitivity to UV Light and/or UV-induced-Skin Damage and/or Tanning Ability; Androgenic Alopecia; Psoriasis; Atopic Dermatitis and/or Eczema; or Latex Allergy, or a subset of the aforementioned phenotypes, such as at least 1, 2, 3, or 4 of the following phenotypes: Melanoma; Non-melanoma Skin Cancer; Sensitivity to UV Light and/or UV-induced Skin Damage and/or Tanning Ability; or Androgenic Alopecia, of which the risk or predisposition to the phenotype is determined.

The Gastroenterology Panel can be used to determine the risk or predisposition for a gastroenterologic-related phenotype, disease or condition of an individual with one or more of the following test results or symptoms: abnormal endoscopy (e.g., upper endoscopy, colonoscopy, virtual colonoscopy, sigmoidoscopy, capsule endoscopy); abnormal blood test; abdominal tenderness upon physical exam; fecal occult blood; Icterus; abdominal pain; abdominal tenderness; nausea; vomiting; diarrhea; and constipation. The Gastroenterology Panel may also be used to screen an individual with a family or medical history of one or more of the following diseases or conditions: inflammatory bowel disease (such as Crohn Disease or Ulcerative Colitis), colorectal cancer, irritable bowel syndrome, gastroesophageal reflux disease, peptic ulcer disease, gastric cancer, liver cancer, and pancreatic cancer. In some cases, an individual with a specific past or present diet may be screened using the Gastroenterology Panel. For example, an individual with a history of consuming a diet containing smoked foods may be tested using the Gastroenterology Panel. In some cases, the Gastroenterology Panel may be used to test an individual who is a current or previous tobacco smoker and/or alcohol drinker and/or illicit drug user. Risks for all of the conditions in the panel, or a subset of the conditions may be determined instead. For example, risks or predisposition of an individual for phenotypes, such as at least 1, 2, 3, 4, 5, 6, 7, 8, 9, or 10 of the following phenotypes: Colorectal Cancer; Peptic Ulcer Disease; Barrett's Esophagus from Gastroesophageal Reflux Disease (GERD); Gastric Cancer; Susceptibility to Gastrointestinal Tract Infections (including but not limited to Enteritis and/or Gastroenteritis); Irritable Bowel Syndrome; Crohn Disease; Ulcerative Colitis; Celiac Disease; or Viral Hepatitis Susceptibility, may be determined. A Gastroenterology Panel can also determine the risk or predisposition of a subset of the aforementioned phenotypes, such as at least 1, 2, 3, or 4 of the following phenotypes: Colorectal Cancer; Peptic Ulcer Disease; Barrett's Esophagus from Gastroesophageal Reflux Disease (GERD); or Gastric Cancer.

Individuals, such as those choosing the Gastroenterology Panel or with results indicating irritable bowel syndrome, or have an abnormal gastrointestinal radiologic exam, abdominal tenderness on physical exam, gastrointestinal biopsy indicative of inflammatory bowel disease, may be interested in the Inflammatory Bowel Disease Panel. Individuals selecting such a panel may be in an urban-industrial societal environment, of Jewish heritage; a current or former tobacco or cigarette smoker, have passive exposure to tobacco or cigarette smoke, or may be suffering from abdominal pain, diarrhea, constipation, weight loss, nausea, vomiting, skin lesions, or any combination thereof. The individual may have a family or medical history of one or more of the following diseases or conditions: Crohn disease and/or ulcerative colitis and/or irritable bowel syndrome and/or celiac disease and/or autoimmune disorder.

The individual can choose to have the risk or predisposition of an individual for phenotypes, such as at least 1, 2, 3, 4, 5, 6, or 7 of the following phenotypes: Crohn Disease; Ulcerative Colitis; Medication Dosage and/or Sensitivity and/or Adverse Reactions and/or Choice for Crohn Disease; Medication Dosage and/or Sensitivity and/or Adverse Reactions and/or Choice for Ulcerative Colitis; Time to Recurrence of Inflammatory Bowel Disease after Medical and/or Surgical Therapy; Symptomatology and/or Disease Location and/or Severity with Crohn Disease; or Location and/or Severity of Ulcerative Colitis, be determined. The Inflammatory Bowel Disease Panel can determine the risk or predisposition of a subset of the aforementioned phenotypes, such as at least 1, 2, 3, or 4 of the following phenotypes: Crohn Disease; Ulcerative Colitis; Medication Dosage and/or Sensitivity and/or Adverse Reactions and/or Choice for Crohn Disease; or Medication Dosage and/or Sensitivity and/or Adverse Reactions and/or Choice for Ulcerative Colitis.

Individuals, such as those choosing the Gastroenterology Panel or with an abnormal gastrointestinal radiologic exam or abdominal tenderness on physical exam may be interested in the Gastrointestinal Disease of Unknown Etiology Panel. The individual selecting such a panel may have an anxiety-prone, rumination-prone and/or worrisome personality, or may be suffering from abdominal pain, diarrhea, constipation, weight loss, nausea, vomiting, or any combination thereof. The individual may have a family or medical history of one or more of the following diseases or conditions: Crohn disease and/or ulcerative colitis, irritable bowel syndrome, celiac disease, or autoimmune disorder. The individual may choose to have the risk or predisposition of an individual for phenotypes, such as at least 1, 2, 3, 4, 5, 6, or 7 of the following phenotypes: Crohn Disease; Ulcerative Colitis; Celiac Disease; Irritable Bowel Syndrome; Porphyria; Endometriosis; or Depression and/or Seasonal Affective Disorder. The Gastrointestinal Disease of Unknown Etiology Panel can determine the risk or predisposition of a subset of the aforementioned phenotypes, such as at least 1, 2, 3, or 4 of the following phenotypes: Crohn Disease; Ulcerative Colitis; Celiac Disease; or Irritable Bowel Syndrome.

Some individuals may be tested using a neurology panel, such as Neurology Panel or Neurologic Disease of Unknown Etiology Panel. The Panels may be used to test an individual with an abnormal result from one or more of the following: neurologic physical examination; brain radiologic examination; or spinal radiologic examination. An individual suffering from one or more of the following symptoms may be screened using either panels: a neurodegenerative disorder, weakness, neuropathy, myopathy, ataxia, movement abnormality, headache, dizziness, vertigo, syncope, and presyncope. Neurologic Disease of Unknown Etiology Panel may also be used to test an individual suffering from one or more: weakness, paresthesia, hemiplegia, and paraplegia. Neurology Panel may be used to test an individual with a family or medical history of one or more: dementia, Alzheimer's Disease, stroke, multiple sclerosis, Parkinson's Disease, motor neuron disease, and neurodegenerative disorder. Neurologic Disease of Unknown Etiology Panel may be used to test an individual with a family history of a specific disease (e.g., a disease or condition described herein with respect to Neurologic Disease of Unknown Etiology Panel) or with a medical history of neurological conditions of unknown etiology. Individuals may select an entire panel, or a subset thereof. For example, an individual may select the Neurology Panel and choose to have the risk or predisposition of an individual for phenotypes, such as at least 1, 2, 3, 4, 5, 6, 7, 8, or 9 of the following phenotypes: Alzheimer's Disease; Stroke (CVA); Headache including Migraine Headaches and/or Cluster Headaches; Lumber Disc Disease; Seizures and/or Epilepsy; Parkinson Disease; Multiple Sclerosis; Myopathies and/or Muscular Atrophy and/or Muscular Dystrophy and/or Neuropathies and/or Charcot-Marie-Tooth Disease; or Motor Neuron Disease including but not limited to Amyotrophic Lateral Sclerosis, be determined. Alternatively, a subset of the aforementioned phenotypes, such as at least 1, 2, 3, or 4 of the following phenotypes: Alzheimer's Disease; Stroke (CVA); Headache including Migraine Headaches and/or Cluster Headaches; or Lumber Disc Disease, may be determined.

An individual may also be interested in Neurology Panel along with Neurologic Disease of Unknown Etiology Panel, or Neurologic Disease of Unknown Etiology Panel alone, and can select all of the phenotypes, or a subset, such as at least 1, 2, 3, 4, 5, 6, 7, 8, 9, 10, 11, 12, 13, or 14 of the following phenotypes: Myopathies and/or Muscular Atrophy and/or Muscular Dystrophy and/or Neuropathies and/or Charcot-Marie-Tooth Disease; Parkinson Disease; Multiple Sclerosis; Ataxia and/or Dystonia and/or Chorea and/or Tremor and/or Tic; Motor Neuron Disease including but not limited to Amyotrophic Lateral Sclerosis; Hemiplegia and/or Paraplegia; Neuromuscular Junction Disorders; Seizures and/or Epilepsy; Huntington's Disease; Dysautonomia; Stroke (CVA); Headache including Migraine Headaches and/or Cluster Headaches; Prion Diseases including but not limited to Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru; or Alzheimer's Disease and/or Dementia. A Neurologic Disease of Unknown Etiology Panel can determine the risk or predisposition of a subset of the aforementioned phenotypes, such as at least 1, 2, 3, 4, 5, or 6 of the following phenotypes: Myopathies and/or Muscular Atrophy and/or Muscular Dystrophy and/or Neuropathies and/or Charcot-Marie-Tooth Disease; Parkinson Disease; Multiple Sclerosis; Ataxia and/or Dystonia and/or Chorea and/or Tremor and/or Tic; Motor Neuron Disease including but not limited to Amyotrophic Lateral Sclerosis; or Hemiplegia and/or Paraplegia.

An individual choosing Neurology Panel and/or Neurologic Disease of Unknown Etiology Panel may also be interested in the Multiple Sclerosis Panel, Alzheimer's Disease Panel, Parkinson Disease Panel, Seizure and Epilepsy Panel, Stroke Panel, or any combination thereof. For example, an individual who has a high risk of Alzheimer's Disease, such as determined by Neurology Panel, may be interested in the Alzheimer's Disease Panel. Alternatively, the individual may be interested in both panels concurrently, or just the Alzheimer's Disease Panel. For example, the individual may have had an abnormal neuropsychological evaluation, abnormal mini mental state examination (MMSE), abnormal functional neuroimaging (including but not limited to SPECT, PET, and/or PiB PET), abnormal cerebrospinal fluid analysis for amyloid β and/or tau proteins, or any combination thereof. The individual may be at least approximately 30, 35, 40, 45 or 50 years of age, possess memory abnormalities, memory loss and/or cognitive abnormalities. The individual may have a medical history or family history of Alzheimer's Disease and/or dementia, increasing memory problems with age, head trauma, traumatic brain injury, or any combination thereof.

Individuals may select the Alzheimer's Disease Panel to determine the risk or predisposition of an individual for phenotypes, such as at least 1, 2, 3, or 4 of the following phenotypes: Alzheimer's Disease; Prognosis and/or Symptomatology and/or Rate of Cognitive Decline with Alzheimer's Disease; Metabolism and/or Effectiveness and/or Dose and/or Choice and/or Adverse Reactions with Medications used to Treat and/or Delay the Onset of Alzheimer's Disease; or Age of Onset of Alzheimer's Disease. The Alzheimer's Disease Panel can determine the risk or predisposition of a subset of the aforementioned phenotypes, such as at least 1, 2, or 3 of the following phenotypes: Alzheimer's Disease; Prognosis and/or Symptomatology and/or Rate of Cognitive Decline with Alzheimer's Disease; or Metabolism and/or Effectiveness and/or Dose and/or Choice and/or Adverse Reactions with Medications used to Treat and/or Delay the Onset of Alzheimer's Disease. Individuals with or without a current or prior diagnosis of Alzheimer's disease or dementia may also be interested in other genetic links to phenotypes, and their risk or predisposition to those phenotypes, that are related to Alzheimer's disease or dementia, and thus may also be interested in the Alzheimer's disease Panel, for example.

An individual interested in the Multiple Sclerosis Panel, for example, an individual who has a high risk of Multiple Sclerosis, such as determined by Neurology Panel, or an individual who may have had an abnormal brain and/or spinal radiologic exam or abnormal neurologic physical exam may select the Multiple Sclerosis Panel. Individuals with or without a current or prior diagnosis of MS may also be interested in other genetic links to phenotypes, and their risk or predisposition to those phenotypes, that are related to MS, and thus may also be interested in the MS Panel, for example. The individual may have decreased UV exposure, decreased vitamin D production, live farther from the equator, be of Caucasian ethnicity, or be a current or former tobacco smoker. The individual may experience stress, weakness, incoordination, sudden blindness, or any combination thereof. The individual may have a medical history or family history of Multiple Sclerosis, autoimmune disease, viral and/or bacterial infection (including but not limited to human endogenous retrovirus, Epstein-Barr virus, varicella zoster virus, spirochetal bacteria, or Chlamydophila pneumoniae), or any combination thereof. The individual may choose to determine the risk or predisposition of all the conditions listed in

Individuals may select the Multiple Sclerosis Panel, which can be used to determine the risk or predisposition for phenotypes such as all or at least 1, 2, 3, or 4 of the following phenotypes: Multiple Sclerosis; Effectiveness and/or Metabolism and/or Dosing and/or Choice and/or Sensitivity and/or Adverse Reactions of Medications used to Treat Multiple Sclerosis; Disease Progression and/or Relapses with Multiple Sclerosis; or Thrombophilia and/or Thromboembolic Disease. A Multiple Sclerosis Panel can determine the risk or predisposition of a subset of the aforementioned phenotypes, such as at least 1, 2, or 3 of the following phenotypes: Multiple Sclerosis; Effectiveness and/or Metabolism and/or Dosing and/or Choice and/or Sensitivity and/or Adverse Reactions of Medications used to Treat Multiple Sclerosis; or Disease Progression and/or Relapses with Multiple Sclerosis.

Individual with a high risk of MS, or others with a family history of autoimmune diseases, may be interested in the Autoimmune Panel. Individuals may select the Autoimmune Panel, which can be used to determine the risk or predisposition of an individual for phenotypes, such as at least 1, 2, 3, 4, 5, 6, 7, 8, 9, 10, 11, or 12 of the following phenotypes: Systemic Lupus Erythematosus (SLE); Crohn Disease; Celiac Disease; Rheumatoid Arthritis; Multiple Sclerosis; Ankylosing Spondylitis; Graves' Disease; Myasthenia Gravis; Psoriasis; Diabetes Mellitus, Type I and/or Mature Onset Diabetes of the Young; Systemic Sclerosis; or Guillain-Barré Syndrome. An Autoimmune Panel can determine the risk or predisposition of a subset of the aforementioned phenotypes, such as at least 1, 2, 3, 4, 5, 6, or 7 of the following phenotypes: Systemic Lupus Erythematosus (SLE); Crohn Disease; Celiac Disease; Rheumatoid Arthritis; Multiple Sclerosis; Ankylosing Spondylitis; or Graves' Disease. Individuals with or without a current or prior diagnosis of any autoimmune disorder or diseases may also be interested in other genetic links to phenotypes, and their risk or predisposition to those phenotypes, that are related to an autoimmune disorder or disorder, and thus may also be interested in the Autoimmune Panel, for example.

An individual choosing Neurology Panel or Neurologic Disease of Unknown Etiology Panel may also be interested in the Parkinson Disease Panel. For example, an individual who has a high risk of Parkinson Disease, such as determined by Neurology Panel may be interested in the Parkinson Disease Panel. For example, the individual may have had an abnormal neurological physical exam, abnormal Unified Parkinson's Disease Rating Scale, abnormal Hoehn and Yahr scale, abnormal Schwab and England Activities of Daily Living Scale, or any combination thereof. Individuals with or without a current or prior diagnosis of Parkinson disease may also be interested in other genetic links to phenotypes, and their risk or predisposition to those phenotypes, that are related to Parkinson disease, and thus may also be interested in the Parkinson Disease Panel, for example. The individual may have been exposed to pesticides, herbicides, fungicides, insecticides, or combinations thereof or may currently or formerly have problems with substance abuse. The individual may possess one or more of the following symptoms: tremors, rigidity, Bradykinesia and/or akinesia, gait and/or postural disturbance, fatigue, speech or swallowing disturbances, shuffling of feet during ambulation, and lack of swing in the arms during ambulation. The individual may have a medical history or family history of Parkinson disease, head trauma, traumatic brain injury, or a combination thereof. An individual may choose to have the risk or predisposition of an individual for phenotypes, such as at least 1, 2, 3, or 4 of the following phenotypes: Parkinson Disease; Symptomatology with Parkinson Disease; Metabolism and/or Dose and/or Choice and/or Adverse Reaction and/or Effectiveness of Medications used to Treat Parkinson Disease; or Age at onset of Parkinson Disease, determined.

An individual choosing Neurology Panel or Neurologic Disease of Unknown Etiology Panel may also be interested in the Seizure Panel. For example, an individual who has a high risk of Seizures, such as determined by Neurologic Disease of Unknown Etiology Panel, may be interested in the Seizure Panel. For example, the individual may have had an abnormal EEG. The individual may have a history of substance abuse or suffer from involuntary change in body movement or function, sensation, awareness, or behavior, presyncope and/or syncope, loss of memory, sensation of unpleasant odor, rapid blinking or staring into space, or any combination thereof. The individual may have a medical history or family history of epilepsy and/or seizure disorder or cardiac arrhythmia. The individual may have the risk or predisposition of an individual for phenotypes, such as at least 1 or 2 of the following phenotypes: Seizures and/or Epilepsy; Antiepileptic Medication Response and/or Effectiveness; or Sensitivity to and/or Dosage Required of Antiepileptic Medication, be determined.

The Mouth and Dental Panel may be used to test an individual with one or more of the following: a disease of the oral cavity; abnormal condition of the oral cavity; an abnormal dental physical or radiologic examination; an abnormal number of teeth; an abnormal distribution of teeth; gum irritation; mouth irritation; past or current bleeding gums; past or current bleeding in the mouth or oral cavity, or a possible syndromic condition. The Mouth and Dental Panel may also be useful to individuals with a family or medical history of one or more: peridontitis; gingival disease; and possible syndromic condition. In some cases, the Mouth and Dental Panel may be used to test an individual with a history of abnormal response to a pain medication or drug (e.g., local or general anesthesia). In some cases, the Mouth and Dental Panel may be used to test an individual with a family history of abnormal number of teeth and/or abnormal distribution of teeth. The Mouth and Dental Panel may also be used in order to help determine an individual's sensitivity and/or response to exposure to mercury. Results from such test may inform decisions relating to dental care, such as whether to remove fillings or what type of dental restorative materials (e.g., mercury amalgam, mercury, metals, composites, cements) or implants are used on an individual. For example, mercury amalgam fillings may be avoided if an individual tests positive for increased susceptibility to mercury poisoning. The tests may also be analyzed in conjunction with results from other panels, or subsets thereof, for example, a panel that tests risks for allergies. Risks for all of the conditions in the panel, or a subset of the conditions may be determined. For example, the risk or predisposition of an individual for phenotypes, such as at least 1, 2, 3, 4, 5, 6, or 7 of the following phenotypes: Periodontitis; Gingival Disease; Dental Abnormalities; Analgesic Effectiveness and/or Sensitivity to Pain Medicine and/or Dosage of Pain Medicine Required for Analgesic Effect; Nitrous Oxide Sensitivity; Sensitivity and/or Toxicity and/or Response to Mercury; or Anesthesia Requirements for Proper Sedation, may be determined. A Mouth & Dental Panel can determine the risk or predisposition of a subset of the aforementioned phenotypes, such as at least 1, 2, or 3 of the following phenotypes: Periodontitis; Gingival Disease; or Dental Abnormalities.

An endocrinology-related panel, or subset thereof, may be used to test an individual for his or her risk or predisposition for hormonal imbalances, disorders, diseases and other endocrine conditions. In some cases, a panel directed to endocrinology-related conditions may be used to test individuals for his or her risk or predisposition for gender identity confusion or ambiguity. For example, an individual with gender identity concerns (e.g., gender dysphoria, gender identity disorder), ambiguous genitalia, abnormal hormone levels or a family history of gender identity disorder may be tested for his or her risk or predisposition for sex reversal or hypogonadism, or other endocrinology-related conditions. The Endocrinology Panel can determine the risk or predisposition of an individual for phenotypes, such as at least 1, 2, 3, 4, 5, 6, 7, 8, or 9 of the following phenotypes: Height; Obesity or Leanness (including but not limited to Weight, BMI, Waist Circumference, Adiposity, and/or Fat Distribution); Diabetes Mellitus, Type II and/or Insulin Resistance; Diabetes Mellitus, Type I and/or Mature Onset Diabetes of the Young; Graves' Disease; Polycystic Ovary Syndrome; Adrenal Hyperplasia and/or Cushing's Syndrome; Primary and/or Secondary Sex Characteristics and/or Sex Reversal and/or Hypogonadism; or Hypogonadism. An Endocrinology Panel can determine the risk or predisposition of a subset of the aforementioned phenotypes, such as at least 1, 2, 3, 4, or 5 of the following phenotypes: Height; Obesity or Leanness (including but not limited to Weight, BMI, Waist Circumference, Adiposity, and/or Fat Distribution); Diabetes Mellitus, Type II and/or Insulin Resistance; or Diabetes Mellitus, Type I and/or Mature Onset Diabetes of the Young; Graves' Disease.

An endocrinology-related panel (or subset thereof) may also be used to test for an individual's risk or predisposition for diabetes. An individual may be tested for his or her risk or predisposition for diabetes or diabetes-related condition (e.g., diabetes mellitus, type I (DM I) diabetes mellitus, type II (DM II); insulin resistance; predisposition for blood glucose level; mature onset diabetes in young people; etc.), particularly if an individual has an abnormal blood glucose level or abnormal glucose tolerance test, has a family or personal medical history of DM I or DM II, obesity, or overweight condition, or has symptoms associated with diabetes such as polydipsia (excessive thirst and/or polyuria (excessive urination)). A positive result for a risk or predisposition to an initial condition that is diabetes or related to diabetes may lead to a test for a reflex condition for a disease or disorder commonly associated with diabetes, such as diabetic retinopathy, diabetic neuropathy, diabetic nephropathy, and/or coronary heart disease. For example, an individual with a family history of DM II and/or manifests a symptom of excessive thirst or excessive urination may test positive for his or her risk or predisposition for DM II, which would then result in a test (or report) for the reflex condition of one or more: diabetic retinopathy, diabetic neuropathy, diabetic nephropathy, and coronary heart disease associated with diabetes, e.g., the Endocrinology Panel. Other reflex conditions that may be tested may include one or more: risk or predisposition for suffering from DM II at a certain age (or indicator of age of onset of DM II), predisposition to metabolize a diabetes medication, predisposition to have adverse reaction to a diabetes medication predisposition for glycemic control with diabetes, predisposition for a certain body mass index (BMI) with diabetes, or risk or predisposition for other condition, e.g. the Endocrinology Pane. Similarly, if an individual tests positive for risk or predisposition for the condition of diabetes mellitus, type I, the individual may be tested for one or more of the following reflex conditions: diabetic retinopathy, diabetic neuropathy, diabetic nephropathy, predisposition for onset of DM I at a certain age, discrepancy between Hemoglobin A1c (Hb A1c) and clinical state, or other DM I-related condition, see, e.g., the Endocrinology Panel. In another example, an adult or child or other individual may test positive for predisposition for obesity and leanness (including indicator of BMI, waist circumference, and fat distribution) and the condition reflexively tested would be his or her risk for DM II (e.g., the Endocrinology Panel).

A diabetes mellitus, type I, panel (e.g., the Diabetes Mellitus, Type I, Panel), or subset thereof, or a diabetes mellitus, type II panel (e.g., the Diabetes Mellitus, Type II, Panel), may also be used to test an individual for his or her risk or predisposition for DM I or DM II. An individual with a family or personal medical history of DM II, of dysglycemia, impaired glucose tolerance, and/or impaired fasting glucose (or other pre-diabetic state), of obesity, of being overweight, of hypertension, of PCOS, or with a personal medical history of metabolic syndrome or disorder of abnormal lipid levels may be tested with a DM II panel, or subset thereof. For example, an individual may choose to have the risk or predisposition of an individual for phenotypes, such as at least 1, 2, 3, 4, 5, 6, 7, 8, 9, 10, 11, 12, or 13 of the following phenotypes: Diabetes Mellitus, Type II and/or Insulin Resistance; Metabolism and/or Response and/or Sensitivity and/or Choice and/or Dose of Medications to Treat Diabetes Mellitus; Coronary Heart Disease in Type II Diabetics; Diabetic Nephropathy with Diabetes Mellitus, Type II, including but not limited to End-Stage Renal Disease; Diabetic Neuropathy with Diabetes Mellitus, Type II; Diabetic Retinopathy with Diabetes Mellitus, Type II; Peripheral Arterial Disease; Exercise Tolerance and/or Optimal Exercise Regimen and/or Athletic Training Regimen for Weight Management and/or To Increase Insulin Sensitivity; Change in Body Fat and/or Lipid Levels with Specific Diets and/or with Exercise; Discrepancy Between Hb A1c Measurement and Clinical State of Diabetic Patient; BMI and/or Waist Circumference with Diabetes Mellitus, Type II; Lipid Levels with Increased BMI and/or Obesity; or Age of Onset of Diabetes Mellitus, Type II, be determined. A Diabetes Mellitus (Type II) Panel can determine the risk or predisposition of a subset of the aforementioned phenotypes, such as at least 1, 2, 3, 4, 5, 6, or 7 of the following phenotypes: Diabetes Mellitus, Type II and/or Insulin Resistance s; Metabolism and/or Response and/or Sensitivity and/or Choice and/or Dose of Medications to Treat Diabetes Mellitus; Coronary Heart Disease in Type II Diabetics; Diabetic Nephropathy with Diabetes Mellitus, Type II, including but not limited to End-Stage Renal Disease; Diabetic Neuropathy with Diabetes Mellitus, Type II; Diabetic Retinopathy with Diabetes Mellitus, Type II; or Peripheral Arterial Disease.

An individual with symptoms such as polyuria, dehydration, polydipsia (excessive thirst), numbness of extremities, abnormal vision, tingling in the extremities, or weight loss may also be tested with a DM II panel, or subset thereof. Individuals with or without a current or prior diagnosis of diabetes mellitus, type II may also be interested in other genetic links to phenotypes, and their risk or predisposition to those phenotypes, that are related to diabetes mellitus, type II may also be interested in the DM II Panel. An individual with an abnormal test result, e.g., abnormal fasting plasma glucose, abnormal glucose tolerance test, or BMI greater than 25; or who has a high-sugar date or does little-to-no exercise may also be tested for DM II. A DM II panel, or subset thereof, may also test for an individual's metabolism of, or reaction to, a diabetes medication (e.g., Metformin, sulfonylureas, insulins, thiazolinediones or other medication listed in the Diabetes Mellitus, (Type II) Panel). Other conditions that may be tested with a DM II panel, or subset thereof include: predisposition for exercise tolerance, disposition for diabetes-related disease (e.g., retinopathy, nephropathy, neuropathy, etc.), predisposition for a certain BMI, predisposition for certain lipid levels, or other condition provided in the Diabetes Mellitus, (Type II) Panel. In some cases, a DM II panel may be used to test an individual for a set of two or more risks (or predisposition), for example, such set may include one of the following sets of risks: risk for DM II and predisposition for metabolism of a diabetes medication (e.g., Metformin, Sulfonylurea, insulin, etc.); risk of DM II and predisposition for exercise tolerance; risk of DM II and risk of diabetic retinopathy with DM II; risk of DM II and risk of diabetic nephropathy with DM II; risk of DM II and risk of diabetic neuropathy with DM II; risk of DM II and predisposition for a certain BMI; risk for DM II and risk for increased lipid levels with increased BMI and/or with obesity; risk of diabetic nephropathy with DM II and risk of diabetic retinopathy with DM II; and risk of diabetic neuropathy with DM II and risk of diabetic retinopathy with DM II.

An individual with a family or personal medial history of DM I, mature onset diabetes of the young, mature pre-diabetic state of dysglycemia; of cystic kidney disease or a personal medical history of viral infection (e.g., coxsackie virus, German measles) may be tested for his or her risk for DM I using a DM I panel, see, e.g., the Diabetes Mellitus (Type I) Panel. For example, an individual may choose to have the risk or predisposition of all the phenotypes, or a subset, such as at least such as at least 1, 2, 3, 4, 5, 6, or 7 of the following phenotypes: Diabetes Mellitus, Type I and/or Mature Onset Diabetes of the Young; Diabetic Retinopathy with Diabetes Mellitus, Type I; Diabetic Nephropathy with Diabetes Mellitus, Type I; Diabetic Neuropathy with Diabetes Mellitus Type I; Peripheral Arterial Disease; Age of Onset of Diabetes Mellitus, Type I; or Discrepancy Between Hb A1c Measurement and Clinical State of Diabetic Patient, determined. A Diabetes Mellitus (Type I) Panel can determine the risk or predisposition of a subset of the aforementioned phenotypes, such as at least 1, 2, 3, 4, or 5 of the following phenotypes: Diabetes Mellitus, Type I and/or Mature Onset Diabetes of the Young; Diabetic Retinopathy with Diabetes Mellitus, Type I; Diabetic Nephropathy with Diabetes Mellitus, Type I; Diabetic Neuropathy with Diabetes Mellitus Type I; or Peripheral Arterial Disease.

Similarly, an individual with certain symptoms (e.g., polyuria, dehydration, failure to thrive, weight loss, polydipsia, abnormal vision, or other symptom associated with DM I, such as a symptom provided in FIG. 21) or with a specific test result (e.g., abnormal fasting plasma glucose level, abnormal glucose tolerance test, etc.) or who has never been breastfed may also be tested with a DM I panel, or subset thereof. In some cases, a DM I panel may be used to test an individual for a set of two or more risks (or predisposition), for example, such set may include one of the following sets of risks: risk for DM I and risk of diabetic neuropathy with DM I; risk of DM I and risk of diabetic retinopathy with DM I; risk of DM I and risk of diabetic nephropathy with DM II; risk of diabetic retinopathy with DM I and risk of diabetic neuropathy with DM I; risk of DM I and risk of other condition provided in the Diabetes Mellitus (Type I) Panel.

An endocrinology panel (or subset thereof) may be used to test for other endocrine-related disorders such as conditions related to the thyroid, to the adrenal gland, or to the ovary. For example, a woman or girl with a family or personal medical history of polycystic ovary syndrome (including suspected, confirmed, or presumed diagnoses), or with an abnormal result from a test for her hormone levels, may be tested with an endocrinology panel for her risk or predisposition for polycystic ovary syndrome (PCOS). If she tests positive for the risk for the initial condition of PCOS, she may be reflexively tested for or analyzed for her risk or predisposition for a certain ovulatory response to a treatment for PCOS (e.g., metformin) or for hirsutism associated with PCOS. The individual may also be tested with a PCOS Panel. The individual may select all the phenotypes, or a subset, such as at least 1, 2, 3, or 4 of the following phenotypes: Polycystic Ovary Syndrome; Ovulatory Response to Metformin Treatment of Polycystic Ovary Syndrome; Symptomatology with Polycystic Ovary Syndrome; or Metabolic Syndrome and/or Impaired Fasting Glucose with Polycystic Ovary Syndrome.

In another example, an individual with a family or personal medical history of thyroid abnormalities may be tested with an endocrinology panel (or subset thereof) for his or her risk or predisposition for a thyroid disease or disorder; and with a positive risk would be reflexively tested or analyzed for his or her risk of ophthalmopathy with Graves Disease and/or his or her risk or predisposition for Graves Disease (severity, age of onset). Other examples are provided in the Endocrinology Panel.

In some cases, an endocrinology panel may be used to test an individual for a set of two or more risks (or predisposition), for example, such set may include one of the following sets of risks his or her risk for DM I and his or her risk for a thyroid disease; his or her risk for DM II and his or her risk for obesity; his or her risk for DM II and his or her risk for thyroid disease; her risk for DM II and her risk for PCOS; her risk for thyroid disease and her risk for PCOS; her risk for DM I and her risk for PCOS; his or her predisposition for height and his or her predisposition for DM II; his or her risk for DM I and his or her risk for obesity; his or her risk of Cushing's Syndrome and his or her risk for DM II; and his or her risk for DM II and his or her risk for thyroid cancer.

In some cases, an endocrinology panel may show an individual with a high risk or predisposition to a thyroid condition, and may choose to be tested with a Thyroid Panel. Individuals may also choose to have a Thyroid Panel test if they have an abnormal thyroid function test, abnormal thyroid radiologic exam, tachycardia or bradycardia, or be postpartum. Individuals with or without a current or prior diagnosis of thyroid disease or a thyroid disorder may also be interested in other genetic links to phenotypes, and their risk or predisposition to those phenotypes, that are related to a thyroid disease or a thyroid disorder, and thus may also be interested in the Thyroid Panel, for example. An individual may have an abnormal heart rate, sensation of being hot or cold, weight loss or weight gain, change in quantity of food eaten, depression or mania, anxiety, lethargy, or a combination thereof. An individual who chooses the Thyroid Panel may also have a personal or family medical history of hyperthyroidism, hypothyroidism, Hashimoto's thyroiditis, thyroid cancer, autoimmune disorder, amiodarone medication, or a combination thereof. An individual may choose to have the risk or predisposition of an individual for phenotypes, such as at least 1, 2, 3, 4, or 5 of the following phenotypes: Graves' Disease; Hypothyroidism; Hashimoto Thyroiditis; Ophthalmopathy with Graves' Disease; Thyroid Cancer; or Age of Onset and/or Severity of Graves' Disease, be determined. The Thyroid Panel can also be used to determine the risk or predisposition of a subset of the aforementioned phenotypes, such as at least 1, 2, 3, or 4 of the following phenotypes: Graves' Disease; Hypothyroidism; Hashimoto Thyroiditis; or Ophthalmopathy with Graves' Disease.

Individuals interested in the Obesity Panel may include those interested in or who have already selected panels described herein, such as the Exercise, Fitness, and Athletic Training Panel and the Dietary, Nutrition, and Weight Management Panel. Individuals interested in the Endocrinology Panel, Diabetes (Type II) Panel and others may also be interested in the Obesity Panel. Individuals with a BMI greater than 25, excessive calorie consumption, or sedentary lifestyle, insufficient sleep, decreased variability in ambient temperature, pregnancy at a later age, ingestion of endocrine disrupters, recent weight gain or fear of weight gain, or any combination thereof, may be interested in the Obesity Panel. Individuals with or without a current or prior diagnosis of being overweight or obese may also be interested in other genetic links to phenotypes, and their risk or predisposition to those phenotypes, that are related to being overweight or obese, and thus may also be interested in the Obesity Panel, for example. Individuals experiencing fatigue, dyspnea, weakness, pain, or any combination thereof, may also be interested in the Obesity Panel. Individuals with a personal or family history of being overweight or obese, have exercise intolerance, sleep apnea, or any combination thereof, may also want to be tested with the Obesity Panel. The Obesity Panel can determine the risk or predisposition of an individual for phenotypes, such as at least 1, 2, 3, 4, 5, 6, 7, or 8 of the following phenotypes: Obesity or Leanness (including but not limited to Weight, BMI, Waist Circumference, Adiposity, and/or Fat Distribution); Diabetes Mellitus, Type II and/or Insulin Resistance; Change in Body Fat and/or Lipid Levels with Specific Diets and/or with Exercise; Exercise Tolerance and/or Optimal Exercise Regimen and/or Athletic Training Regimen for Weight Management; Amount of Effort Needed to Lose Weight; Amount of Food Consumption (including but not limited to Intake of Total Energy and/or Dietary Fat and/or Dietary Protein and/or Dietary Carbohydrate); Lipid Levels with Increased BMI and/or Obesity; or Depression and/or Seasonal Affective Disorder. A Obesity Panel can determine the risk or predisposition of a subset of the aforementioned phenotypes, such as at least 1, 2, 3, 4, 5, or 6 of the following phenotypes: Obesity or Leanness (including but not limited to Weight, BMI, Waist Circumference, Adiposity, and/or Fat Distribution); Diabetes Mellitus, Type II and/or Insulin Resistance; Change in Body Fat and/or Lipid Levels with Specific Diets and/or with Exercise; Exercise Tolerance and/or Optimal Exercise Regimen and/or Athletic Training Regimen for Weight Management; Amount of Effort Needed to Lose Weight; Amount of Food Consumption (including but not limited to Intake of Total Energy and/or Dietary Fat and/or Dietary Protein and/or Dietary Carbohydrate).

Individuals concerned about their risk of addictive behavior, for example, because of a family history of addiction (including but not limited to nicotine, alcohol, narcotics and/or medications), psychiatric illness (including but not limited to depression, bipolar spectrum disorder, schizophrenia, OCD, anxiety disorder, and/or panic disorder) may be interested in the Addiction Panel. Individuals that are stuporous, not alert, not oriented, have low self-esteem, been in or are presently in the military service, been in or are presently incarcerated or on parole, past/current addiction (including but not limited to nicotine, alcohol, narcotics and/or medications), suffers/suffered childhood abuse, experience depression, experience anxiety, are suicidal, have strong feelings of guilt, may choose to be tested with the Addiction Panel. The panel may be utilized by addiction centers, rehabilitation programs, 12-step groups and facilities, jails and prisons, addiction recovery groups and/or therapists, eating disorder clinic, nutritionist, interventionist, within corporations and/or government, by religious groups, by medical professionals, or by the individual.

For example, the Addiction Panel can be used to determine the risk or predisposition of an individual for phenotypes, such as at least 1, 2, 3, 4, 5, 6, 7, 8, or 9 of the following phenotypes: Nicotine Addiction and/or Nicotine Dependence; Alcoholism, Alcohol Dependence and/or Alcohol Abuse; Opiate and/or Heroin Addiction; Drug Abuse, Dependency & Addiction (Including Cannabis and/or Opiates and/or Heroin and/or Benzodiazepines and/or Cocaine and/or Amphetamines); Habitual Caffeine Consumption and/or Caffeine Addiction; Suicidality; Alcohol Dependence with Co-Morbid Drug Dependence or Major Depression; Binge Drinking; or Stressful Life Events causing Depressive Symptoms and/or Diagnosable Depression and/or Suicidality and/or Anxiety (including but not limited to Mental Vulnerability to Stress and/or Disease). An Addiction Panel can determine the risk or predisposition of a subset of the aforementioned phenotypes, such as at least 1, 2, 3, or 4 of the following phenotypes: Nicotine Addiction and/or Nicotine Dependence; Alcoholism, Alcohol Dependence and/or Alcohol Abuse; Opiate and/or Heroin Addiction; or Drug Abuse, Dependency & Addiction (Including Cannabis and/or Opiates and/or Heroin and/or Benzodiazepines and/or Cocaine and/or Amphetamines). Individuals with or without a current or prior diagnosis of substance abuse, substance dependence, or substance addiction may also be interested in other genetic links to phenotypes, and their risk or predisposition to those phenotypes, that are related to substance abuse, substance dependence, or substance addiction, and thus may also be interested in the Addiction Panel, for example.

Individuals with a high risk of a specific addictive behavior, for example, as determined by using the Addiction Panel, or interested in their risk of addiction to a specific substance, may choose to determine their risk to smoking or alcoholism, by selecting the Smoker's Panel or Drinker's Panel. An individual may select the Smoker's Panel and choose to have the risk or predisposition of an individual for phenotypes, such as at least 1, 2, 3, 4, 5, 6, 7, 8, 9, or 10 of the following phenotypes: Nicotine Addiction and/or Nicotine Dependence; Effectiveness and/or Dosing and/or Choice of Cessation Modality for the Treatment of Nicotine Addiction; Risk of Cancer with Smoking (Including but not Limited to Lung Cancer, Gastric Cancer, Colorectal Cancer, and/or Esophageal Cancer); Risk of Coronary Artery Disease and/or Myocardial Infarction with Smoking; Ease and Likelihood of Quitting Smoking; Quantity and/or Heaviness of Smoking; Chronic Obstructive Pulmonary Disease (COPD); Peripheral Arterial Disease; Macular Degeneration; or Thrombophilia and/or Thromboembolic Disease, be determined. A Smoker's Panel can determine the risk or predisposition of a subset of the aforementioned phenotypes, such as at least 1, 2, 3, 4, or 5 of the following phenotypes: Nicotine Addiction and/or Nicotine Dependence; Effectiveness and/or Dosing and/or Choice of Cessation Modality for the Treatment of Nicotine Addiction; Risk of Cancer with Smoking (Including but not Limited to Lung Cancer, Gastric Cancer, Colorectal Cancer, and/or Esophageal Cancer); Risk of Coronary Artery Disease and/or Myocardial Infarction with Smoking; or Ease and Likelihood of Quitting Smoking. Individuals with or without a current or prior diagnosis of nicotine addiction, smoking addiction, smoking abuse, or smoking dependence may also be interested in other genetic links to phenotypes, and their risk or predisposition to those phenotypes, that are related to nicotine addiction, smoking addiction, smoking abuse, or smoking dependence, and thus may also be interested in the Smoker's Panel, for example.

Some individuals may be interested in the Drinker's Panel, and may choose to have the risk or predisposition of an individual for phenotypes, such as at least 1, 2, 3, 4, 5, or 6 of the following phenotypes: Alcoholism, Alcohol Dependence and/or Alcohol Abuse; Effect of Treatment and/or Withdrawal for Alcohol Dependence (including but not limited to Tremor, Agitation, Anxiety, Food Craving, Weight Change, Movement Abnormalities, and/or Memory Abnormalities); Effectiveness of Twelve-step Facilitation to treat Alcoholism versus Cognitive Behavioral Therapy versus Motivational Enhancement Therapy; Effectiveness and/or Choice and/or Adverse Reactions of Medications used to Treat Alcoholism; Susceptibility to Liver Disease due to Alcohol; Risk of Cancer with Alcohol Consumption; or Chronic Pancreatitis due to Alcohol, be determined. A Drinker's Panel can determine the risk or predisposition of a subset of the aforementioned phenotypes, such as at least 1, 2, 3, 4, or 5 of the following phenotypes: Alcoholism, Alcohol Dependence and/or Alcohol Abuse; Effect of Treatment and/or Withdrawal for Alcohol Dependence (including but not limited to Tremor, Agitation, Anxiety, Food Craving, Weight Change, Movement Abnormalities, and/or Memory Abnormalities); Effectiveness of Twelve-step Facilitation to treat Alcoholism versus Cognitive Behavioral Therapy versus Motivational Enhancement Therapy; Effectiveness and/or Choice and/or Adverse Reactions of Medications used to Treat Alcoholism; or Susceptibility to Liver Disease due to Alcohol. Individuals with or without a current or prior diagnosis of alcoholism, alcohol abuse or alcohol dependence may also be interested in other genetic links to phenotypes, and their risk or predisposition to those phenotypes, that are related to alcoholism, alcohol abuse, or alcohol dependence, and thus may also be interested in the Drinker's Panel, for example.

Psychiatrists, psychologists, internists, therapists, hospitals or clinics, eating disorder centers, the military, the police, governments and governmental agencies, space agencies, schools, daycare centers, or summer camps whose employees or teachers care for or interact with children, health insurance companies, life insurance companies, disability insurance companies, employment insurance companies, businesses or corporations, individuals themselves, or others, may be interested in their risk or predisposition to mental conditions. For example, individuals with an abnormal psychological evaluation, or with current, previous, upcoming or current military service, previous, upcoming or current police service or employment, previous, upcoming or current government service or employment, potential or current employment with a business or corporate, potential or current employment or matriculation at a medical school, hospital, medical center, clinic, or physician's office, potential or current employment with a daycare center, summer camp or school, potential or current matriculation at a school or university, an addiction (including but not limited to nicotine, alcohol, narcotics and/or medications), high risk of an addiction (such as determined by use of the Addiction Panel described herein), or childhood abuse may be interested in getting an Adult Psychiatry Panel. Individuals experiencing anxiety, psychosis, depression, suicidal thoughts, or with a personal or family medical history of depression, suicidality, attention deficit hyperactivity disorder, post-traumatic stress disorder, schizophrenia, bipolar spectrum disorder, psychosis, obsessive compulsive disorder or panic disorder may be tested with the panel. Individuals may be tested for their risk or predisposition of an individual for phenotypes, such as at least 1, 2, 3, 4, 5, 6, 7, 8, 9, 10, 11, or 12 of the following phenotypes: Suicidality; Depression; Seasonal Affective Disorder; Stressful Life Events causing Depressive Symptoms and/or Depression (including but not limited to Mental Vulnerability to Stress and/or Disease); Caffeine Metabolism (including but not limited to Caffeine Consumption's Effect on Sleep); Bipolar Spectrum Disorder; Schizophrenia; Panic Disorder; Obsessive-Compulsive Disorder; Attention Deficit Hyperactivity Disorder; General Anxiety Disorder; or Effect of Stimulant(s) on Cognition, be determined. An Adult Psychiatry Panel can determine the risk or predisposition of a subset of the aforementioned phenotypes, such as at least 1, 2, 3, or 4 of the following phenotypes: Suicidality; Depression; Seasonal Affective Disorder; or Stressful Life Events causing Depressive Symptoms and/or Depression (including but not limited to Mental Vulnerability to Stress and/or Disease).

Individuals with a high risk of a specific behavior or mental condition, for example, as determined by using the Adult Psychiatry Panel, or interested in their risk of a psychiatric condition may be interested in the Depression Panel, Schizophrenia Panel, Bipolar Panel, or Eating Disorder Panel.

For example, an individual, psychiatrist, psychologist, therapist, physician, or other healthcare provider may be interested in the Depression Panel and be interested in finding out their risk or predisposition, or his or her patient's risk, for all the phenotypes, or a subset, such as at least 1, 2, 3, 4, 5, 6, 7, or 8 of the following phenotypes: Depression; Seasonal Affective Disorder; Treatment-Emergent Suicidality during Treatment with Antidepressants; Stressful Life Events causing Depressive Symptoms and/or Diagnosable Depression and/or Suicidality and/or Anxiety (including but not limited to Mental Vulnerability to Stress and/or Disease); Effectiveness and/or Sensitivity and/or Response to Medications used to Treat Depression; Response Rates to Treatment for Depression; Suicidality; Caffeine Metabolism (including but not limited to Caffeine Consumption's Effect on Sleep); or Insomnia and/or Level of Sleepiness. A Depression Panel can determine the risk or predisposition of a subset of the aforementioned phenotypes, such as at least 1, 2, 3, 4, 5, 6, or 7 of the following phenotypes: Depression; Seasonal Affective Disorder; Treatment-Emergent Suicidality during Treatment with Antidepressants; Stressful Life Events causing Depressive Symptoms and/or Diagnosable Depression and/or Suicidality and/or Anxiety (including but not limited to Mental Vulnerability to Stress and/or Disease); Effectiveness and/or Sensitivity and/or Response to Medications used to Treat Depression; Response Rates to Treatment for Depression; or Suicidality.

An individual, psychiatrist, psychologist, therapist, physician, or other healthcare provider can be interested in the Schizophrenia Panel. The individual may have an abnormal psychological evaluation or been born in winter or spring. The individual may have a history of substance abuse and/or substance dependence, childhood abuse and/or trauma, been living in an urban environment, experiencing poverty, racial discrimination, family dysfunction, unemployment, poor housing conditions, or a combination thereof. Individuals with or without a current or prior diagnosis of schizophrenia may also be interested in other genetic links to phenotypes, and their risk or predisposition to those phenotypes, that are related to schizophrenia, and thus for example may be interested in the Schizophrenia Panel. The individual may have a personal or family history of schizophrenia, schizophreniform disorder, depression, anxiety disorder; suicidality, exposure to infection during prenatal stage of development, or any combination thereof. An individual may choose to have the risk or predisposition of all the phenotypes, or a subset, such as at least 1, 2, 3, 4, 5, or 6 of the following phenotypes: Schizophrenia; Degree of Severity of or Symptomology with Schizophrenia; Aggressiveness or Homicidal Behavior with Schizophrenia; Weight Change and/or BMI Change and/or Change in Lipid Levels Associated with Medication used to Treat Schizophrenia; Effectiveness and/or Dose and/or Choice and/or Sensitivity and/or Response and/or Adverse Reactions to Antipsychotic Medications; or Antipsychotic Medication Induced Parkinsonism, be determined.

An individual, psychiatrist, psychologist, therapist, physician, or other healthcare provider can be interested in the Bipolar Panel. The individual may have an abnormal psychological evaluation and be stressed, pregnant or just given birth. The individual may be experiencing mania and/or hypomania, depression, suicidality, agitation, confusion, anxiety, or a combination of such symptoms. Individuals with or without a current or prior diagnosis of bipolar disorder may also be interested in other genetic links to phenotypes, and their risk or predisposition to those phenotypes, that are related to bipolar disorder, and thus may be interested in the Bipolar Panel. The individual may have a personal or family history of childhood anxiety, childhood depression, schizoaffective disorder, bipolar spectrum disorder, manic and/or hypomanic episodes, schizophrenia, eating disorder, premenstrual dysphoric disorder, postpartum depression, postpartum psychosis, or any combination thereof. An individual may choose to have the risk or predisposition of all the phenotypes, or a subset, such as at least 1, 2, 3, 4, 5, 6, 7, 8, or 9 of the following phenotypes: Bipolar Spectrum Disorder; Effectiveness and/or Dose and/or Choice and/or Sensitivity and/or Response and/or Adverse Reactions to Mood Stabilizers and/or Antipsychotic Medications used to Treat Bipolar Disorder; Lithium Response in Mania and/or Bipolar Disorder; Antipsychotic Medication Induced Parkinsonism; Suicidality; Treatment-Emergent Suicidality during Treatment with Antidepressants; Weight Change and/or BMI Change Associated with Antipsychotic Medication; Cognitive Performance with Bipolar Disorder; or Stressful Life Events causing Depressive Symptoms and/or Diagnosable Depression and/or Suicidality and/or Anxiety (including but not limited to Mental Vulnerability to Stress and/or Disease), be determined. A Bipolar Panel can determine the risk or predisposition of a subset of the aforementioned phenotypes, such as at least 1, 2, 3, 4, or 5 of the following phenotypes: Bipolar Spectrum Disorder; Effectiveness and/or Dose and/or Choice and/or Sensitivity and/or Response and/or Adverse Reactions to Mood Stabilizers and/or Antipsychotic Medications used to Treat Bipolar Disorder; Lithium Response in Mania and/or Bipolar Disorder; Antipsychotic Medication Induced Parkinsonism; or Suicidality.

An Individual, rheumatologist, gastroenterologist, internist, psychiatrist, psychologist, therapist, physician; or other healthcare provider may also select the Fibromyalgia Panel, which can be used to determine the risk or predisposition of an individual for phenotypes, such as at least 1, 2, 3, 4, 5, or 6 of the following phenotypes: Fibromyalgia; Severity of Fibromyalgia; Depression and/or Seasonal Affective Disorder; General Anxiety Disorder; Stressful Life Events causing Depressive Symptoms and/or Diagnosable Depression and/or Suicidality and/or Anxiety (including but not limited to Mental Vulnerability to Stress and/or Disease); or Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance). A Fibromyalgia Panel can determine the risk or predisposition of a subset of the aforementioned phenotypes, such as at least 1, 2, 3, or 4 of the following phenotypes: Fibromyalgia; Severity of Fibromyalgia; Depression and/or Seasonal Affective Disorder; or General Anxiety Disorder. Individuals with or without a current or prior diagnosis of fibromyalgia may also be interested in other genetic links to phenotypes, and their risk or predisposition to those phenotypes, that are related to fibromyalgia, and may also be interested in the Fibromyalgia Panel, for example.

An individual, a health care provider, family, eating disorder clinic, hospital, psychiatrist, psychologist, or therapist may be interested in the Eating Disorder Panel. The individual may have a weight of less than approximately 85% of the weight that is normal for the individual's age and height, a body mass index equal to or less than 17.5, an abnormal result from a questionnaire of eating and weight patterns (QEWP), abnormal electrolyte levels, metabolic alkalosis, abnormal psychological evaluation, high cortisol level, low serotonin levels, or any combination thereof. The individual may be a perfectionist, have controlling parent(s), low self-esteem, high self-criticism, environmental stress (including but not limited to sexual abuse and/or physical abuse and/or emotional abuse and/or racism and/or poverty), or any combination thereof. Individuals with or without a current or prior diagnosis of anorexia nervosa or bulimia nervosa may also be interested in other genetic links to phenotypes, and their risk or predisposition to those phenotypes, that are related to anorexia nervosa or bulimia nervosa. The individual may have abnormally low body weight and/or weight loss, amenorrhea, intense fear of gaining weight, binge eating, purging, guilt, suicidality, or any combination thereof. The individual may have a personal or family history of eating disorder(s) (including but not limited to anorexia nervosa and/or bulimia nervosa), sexual abuse, physical abuse, emotional abuse, body dysmorphic syndrome, or any combination thereof. An individual may choose to have the risk or predisposition of an individual for phenotypes, such as at least 1, 2, 3, 4, 5, 6, or 7 of the following phenotypes: Anorexia Nervosa; Bulimia Nervosa; Suicidality; Treatment-Emergent Suicidality during Treatment with Antidepressants; Age of Onset of Eating Disorders; Depression and/or Seasonal Affective Disorder; or Stressful Life Events causing Depressive Symptoms and/or Diagnosable Depression and/or Suicidality and/or Anxiety (including but not limited to Mental Vulnerability to Stress and/or Disease). An Eating Disorder Panel can determine the risk or predisposition of a subset of the aforementioned phenotypes, such as at least 1, 2, or 3 of the following phenotypes: Anorexia Nervosa; Bulimia Nervosa; or Suicidality.

Parents, guardians, schools, or health care mangers of children may be interested in having a child tested for their risk or predisposition to mental or psychiatric conditions with the use of the Pediatric Psychiatry Panel, Autism Panel, Learning and Education Panel, or any combination thereof. The aforementioned panels, entire or a subset thereof, may be used concurrently or sequentially for testing an individual. For example, a Pediatric Psychiatry Panel may be used to test an individual for their risk or predisposition of an individual for phenotypes, such as at least 1, 2, 3, 4, 5, 6, 7, 8, or 9 of the following phenotypes: Intelligence (IQ); Suicidality; Attention Deficit Hyperactivity Disorder; Pervasive Developmental Disorder (including but not limited to Autism, Autism Spectrum Disorder, Asperger Syndrome, and/or Rett Syndrome); Mental Retardation; Effect of Stimulant(s) on Cognition; Novelty Seeking Behavior/Personality; or Stressful Life Events causing Depressive Symptoms and/or Diagnosable Depression and/or Suicidality and/or Anxiety (including but not limited to Mental Vulnerability to Stress and/or Disease); Drug Abuse, Dependency & Addiction (Including Cannabis and/or Opiates and/or Heroin and/or Benzodiazepines and/or Cocaine and/or Amphetamines). A Pediatric Psychiatry Panel can determine the risk or predisposition of a subset of the aforementioned phenotypes, such as at least 1, 2, 3, 4, or 5 of the following phenotypes: Intelligence (IQ); Suicidality; Attention Deficit Hyperactivity Disorder; Pervasive Developmental Disorder (including but not limited to Autism, Autism Spectrum Disorder, Asperger Syndrome, and/or Rett Syndrome); or Mental Retardation. As with all of the panels, these panels can be run on any genetic material from an embryo or fetus, including but not limited to cells from an amniocentesis or chorionic villus sampling (CVS), or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, the amnion, the amniotic sac, the blood of a pregnant female or via peripheral or central blood draw(s) from the pregnant female.

Concurrent with testing a child with the Pediatric Psychiatry Panel, a child may be tested with the Autism Panel, Learning and Education Panel, or both. Alternatively, a child may be tested with either panels or both panels, or subsets the conditions within each panel, after obtaining results from the Pediatric Psychiatry Panel. Other panels described herein may also be used to test the child. For example, a child may have a high risk for having an eating disorder as determined by using the Pediatric Psychiatry Panel, and subsequently, the Eating Disorder Panel may be used to test the child. In another example, the child may have a high risk or autism, such as determined by use of the Pediatric Psychiatry Panel and thus the child may then be tested with the Autism Panel. Alternatively, the child may not have been tested with the Pediatric Psychiatry Panel prior to being tested with the Autism Panel. Furthermore, individuals that are not children may also be tested with the Autism Panel. An individual, adult or child, being tested with the Autism Panel may have had an abnormal psychiatric or psychological evaluation or vaccinations as an infant or child. An individual being tested with the Autism Panel may have difficulty using and/or understanding language, difficulty relating to people, objects, and/or events, repetitive body movements, repetitive behavior patterns, unusual play with toys and/or other objects, difficulty with changes in routine, or any combinations thereof. An individual being tested with the Autism Panel may have a personal or family history of pervasive developmental disorder, mental retardation, or both. Individuals with or without a current or prior diagnosis of autism may also be interested in other genetic links to phenotypes, and their risk or predisposition to those phenotypes, that are related to autism. The risk or predisposition of an individual for phenotypes, such as at least 1, 2, 3, 4, 5, or 6 of the following phenotypes: Autism and/or Autism Spectrum Disorder; Asperger Syndrome; Rett Syndrome; Degree of Language Deficits in Autism; Degree of Social Interactions with Autism; Types of Behavior with Autism; or Mental Retardation, may be determined. An Autism Panel can determine the risk or predisposition of a subset of the aforementioned phenotypes, such as at least 1, 2, 3, 4, or 5 of the following phenotypes: Autism and/or Autism Spectrum Disorder; Asperger Syndrome; Rett Syndrome; Degree of Language Deficits in Autism; or Degree of Social Interactions with Autism.

In another example, the child may have a high risk or predisposition to autism or difficulties with learning, such as determined by use of the Pediatric Psychiatry Panel or the Autism Panel, and thus the child may then be tested with the Learning and Education Panel. Alternatively, the child may not have been tested with the Pediatric Psychiatry Panel or Autism Panel prior to being tested with the Learning and Education Panel. Furthermore, individuals that are not children may also be tested with the Learning and Education Panel. An individual, adult or child, being tested with the Learning and Education Panel may have had or have one or more of the following conditions: an abnormal psychiatric/psychological evaluation, abnormal intelligence quotient evaluation, delayed speech and/or language development, below average reading ability, learning difficulties, frustration, anger, or difficulty using and understanding language. The individual interested in the Learning and Education Panel may also have a personal or family history of dyslexia, attention deficit hyperactivity disorder, autism; Asperger syndrome; Rett syndrome, childhood disintegrative disorder, pervasive developmental disorder, or any combination thereof. The individual may be tested for their risk or predisposition of an individual for phenotypes, such as at least 1, 2, 3, 4, 5, 6, 7, 8, 9, or 10 of the following phenotypes: Pervasive Developmental Disorder (including but not limited to Autism, Autism Spectrum Disorder, Asperger Syndrome, and/or Rett Syndrome); Attention Deficit Hyperactivity Disorder; Dyslexia; Reading Ability and/or Performance; Speech and/or Language Development; Insomnia and/or Level of Sleepiness; Idiopathic Hypersomnia; Narcolepsy; Sleep Apnea; or Effect of Stimulant(s) on Cognition. A Learning & Education Panel can determine the risk or predisposition of a subset of the aforementioned phenotypes, such as at least 1, 2, 3, 4, or 5 of the following phenotypes: Pervasive Developmental Disorder (including but not limited to Autism, Autism Spectrum Disorder, Asperger Syndrome, and/or Rett Syndrome); Attention Deficit Hyperactivity Disorder; Dyslexia; Reading Ability and/or Performance; or Speech and/or Language Development.

Other individuals may be interested in the Infectious Disease Panel, Worldwide Infectious Disease Panel, or Infection Panel. National and International agencies, such as the United States Centers for Disease Control (CDC) and the United Nation's World Health Organization (WHO), may also be interested in these panels. For example, a traveler is planning to travel to a country with a recent flu outbreak, or a malaria or typhoid fever or tuberculosis or Human Immunodeficiency Virus (HIV) infected region, he or she may be interested in their risk of infection. In another example, individuals with abnormal blood test suggestive or indicative of infection; Abnormal erythrocyte sedimentation rate (ESR) and/or C-reactive protein (CRP), abnormal lumbar puncture test results or experiencing fever, malaise, rash, cough, headache, diarrhea, myalgias and/or arthralgias may be interested in these panels. Individuals with current or previous history of sexual activity, occupation exposure to many different people and/or tourists, or any combination thereof; or individuals with a personal or family history of HIV infection, hepatitis, diarrheal illness, tuberculosis, malaria, Meningococcal disease, severe acute respiratory syndrome, West Nile virus, Severe Acute Respiratory Syndrome (SARS) or any combination thereof, may be interested in being tested with one of these panels. Risks for all of the conditions in the panel, or a subset of the conditions may be determined instead.

For example, the risk or predisposition of an individual for phenotypes, such as at least 1, 2, 3, 4, 5, 6, 7, 8, or 9 of the following phenotypes: Human Immunodeficiency Virus (HIV) Infection Susceptibility; Hepatitis C Virus Susceptibility; Meningococcal Disease Susceptibility; Pneumococcal Disease Susceptibility; Susceptibility to Bacteremia and/or Sepsis and/or Severe Sepsis and/or Septic Shock and/or Systemic Inflammatory Response Syndrome; Severe Acute Respiratory Syndrome (SARS) Susceptibility; West Nile Virus Susceptibility; Susceptibility to Gastrointestinal Tract Infections (including but not limited to Enteritis and/or Gastroenteritis); or Viral and/or Bacterial and/or Fungal and/or Parasitic Infections Susceptibility, may be determined. An Infectious Disease Panel can determine the risk or predisposition of a subset of the aforementioned phenotypes, such as at least 1, 2, or 3 of the following phenotypes: Human Immunodeficiency Virus (HIV) Infection Susceptibility; Hepatitis C Virus Susceptibility; or Meningococcal Disease Susceptibility.

Individuals who travel (including but not limited to international travel and/or national travel to disease endemic areas) or who live in countries other than the United States, Canada, Northern Europe or Western Europe, may be more interested in the World Infectious Disease Panel. The individual may have a personal or family history of hepatitis, tuberculosis, malaria, diarrheal illness, Meningococcal disease, Hepatitis C virus, severe acute respiratory syndrome, West Nile virus, Severe Acute Respiratory Syndrome, HIV infection, leprosy, typhoid, Dengue fever, or any combination thereof. The risk or predisposition of an individual for phenotypes, such as at least 1, 2, 3, 4, 5, 6, 7, 8, 9, 10, or II of the following phenotypes: Human Immunodeficiency Virus (HIV) Infection Susceptibility or Resistance; Malaria Susceptibility; Tuberculosis Susceptibility; Leprosy Susceptibility; Typhoid Susceptibility; Dengue Fever Susceptibility; Norovirus Susceptibility (including but not limited to Norwalk Virus Susceptibility); Susceptibility to Gastrointestinal Tract Infections (including but not limited to Enteritis and/or Gastroenteritis); Hepatitis C Virus Susceptibility; Severe Acute Respiratory Syndrome (SARS) Susceptibility; or West Nile Virus Susceptibility. A World Infectious Disease Panel can determine the risk or predisposition of a subset of the aforementioned phenotypes, such as at least 1, 2, 3, 4, 5, or 6 of the following phenotypes: Human Immunodeficiency Virus (HIV) Infection Susceptibility or Resistance; Malaria Susceptibility; Tuberculosis Susceptibility; Leprosy Susceptibility; Typhoid Susceptibility; or Dengue Fever Susceptibility.

Individuals interested in their risk or predisposition to infections or who are suspected of having an infection or who have been diagnosed with an infection or who have acute or chronic infections may also choose the Infection Panel, which can be used to determine the risk or predisposition of an individual for phenotypes, such as at least 1, 2, 3, 4, 5, or 6 of the following phenotypes: Susceptibility to Bacteremia and/or Sepsis and/or Severe Sepsis and/or Septic Shock and/or Systemic Inflammatory Response Syndrome; Severity of Sepsis and/or Severe Sepsis and/or Septic Shock and/or Systemic Inflammatory Response Syndrome (including but not limited to Organ Dysfunction, Organ Injury, Death, and/or Outcome); Source of Infection and/or Type of Bacteria with Bacteremia and/or Sepsis and/or Severe Sepsis and/or Septic Shock and/or Systemic Inflammatory Response Syndrome; Thrombophilia and/or Thromboembolic Disease; Drug Metabolism and/or Choice and/or Sensitivity and/or Adverse Reactions and/or Dosing (Including Pharmacogenomic Analysis for all Pharmaceuticals); Bleeding Diathesis and/or Coagulation Disorders and/or Hemophilia; or Infectious Disease Susceptibility (including but not limited to Human Immunodeficiency Virus (HIV), Hepatitis B Virus (HBV), Hepatitis C Virus (HCV), Norwalk Virus, Meningococcal Disease, Pneumococcal Disease, Severe Acute Respiratory Syndrome, Legionnaire Disease, West Nile Virus, Malaria, Tuberculosis, Leprosy, Atypical Mycobacteria, Typhoid, Dengue Fever, Aspergillosis, Toxoplasmosis, Prion Diseases, Epstein-Barr Virus, Salmonella, Schistosomiasis, Lyme Disease, Herpes Simplex Virus, Gastrointestinal Tract Infections, Fungal Infections, and/or Parasitic Infections.)

An Infection Panel can determine the risk or predisposition of a subset of the aforementioned phenotypes, such as at least 1, 2, 3, 4, or 5 of the following phenotypes: Susceptibility to Bacteremia and/or Sepsis and/or Severe Sepsis and/or Septic Shock and/or Systemic Inflammatory Response Syndrome; Severity of Sepsis and/or Severe Sepsis and/or Septic Shock and/or Systemic Inflammatory Response Syndrome (including but not limited to Organ Dysfunction, Organ Injury, Death, and/or Outcome); Source of Infection and/or Type of Bacteria with Bacteremia and/or Sepsis and/or Severe Sepsis and/or Septic Shock and/or Systemic Inflammatory Response Syndrome; Thrombophilia and/or Thromboembolic Disease; or Drug Metabolism and/or Choice and/or Sensitivity and/or Adverse Reactions and/or Dosing (Including Pharmacogenomic Analysis for all Pharmaceuticals).

Individual interested in their risk or predisposition to infections or infectious diseases, individuals working or living in close quarters with others, or administrators, officials, individuals living within, students of, consultants to, employees of, or candidates for employment of large corporations, agencies, the military, space programs, institutes (e.g. schools, colleges, universities, summer camps), or disaster temporary housing residents or management (such as that set up by Federal Emergency Management Agency (FEMA)) may be interested in the Close Living Quarters Panel, which can be used to determine the risk or predisposition of an individual for phenotypes, such as at least 1, 2, 3, 4, 5, or 6 of the following phenotypes: Norovirus Susceptibility (including but not limited to Norwalk Virus Susceptibility); Meningococcal Disease Susceptibility; Tuberculosis Susceptibility; Susceptibility to Gastrointestinal Tract Infections (including but not limited to Enteritis and/or Gastroenteritis); Hepatitis C Virus Susceptibility; or Human Immunodeficiency Virus (HIV) Infection Susceptibility or Resistance. A Close Living Quarters Panel can determine the risk or predisposition of a subset of the aforementioned phenotypes, such as at least 1, 2, or 3 of the following phenotypes: Norovirus Susceptibility (including but not limited to Norwalk Virus Susceptibility), Meningococcal Disease Susceptibility; or Tuberculosis Susceptibility.

Individuals interested in their risk or predisposition to specific infections may choose the HIV Panel, Viral Hepatitis Panel, Malaria Panel, or a combination thereof. National and International agencies, such as the United States Centers for Disease Control (CDC) and the United Nation's World Health Organization (WHO), may also be interested in these panels. Individuals choosing such panels may have been tested with an (Worldwide) Infectious Disease Panel before deciding to choose the HIV Panel, Viral Hepatitis Panel, Malaria Panel, or a combination thereof. Alternatively, the individual may not have been tested with the Infectious Disease Panel. An individual to be tested with the HIV Panel may have had an HIV positive laboratory test, an abnormal CD4 blood test; or a blood and/or genital exam positive for STDs. An individual to be tested with the HIV Panel may have current, past, or future planned sexual activity, history of intravenous drug use, history of blood transfusion, history of needle stick, history direct contact with someone else's blood, or any combination thereof. The individual may have one or more of the following symptoms: fever, rash, or malaise. The individual may be HIV positive or have a family history with one or more HIV positive family member, family member with history of risky behavior, and/or a family member with direct contact with HIV infected blood or person but remained HIV negative. An individual may have their risk or predisposition of an individual for phenotypes, such as at least 1, 2, 3, 4, 5, 6, or 7 of the following phenotypes: Human Immunodeficiency Virus (HIV) Infection Susceptibility to or Resistance against; Rate of Progression and/or Prognosis with HIV Infection; HIV Medication Metabolism and/or Hypersensitivity and/or Dose and/or Choice of Medication used for Treatment or Prophylaxis; HIV Infection Treatment—Bone Marrow Transplant Donor Eligibility: Bone Marrow Transplant Donor Able to Offer Possible Treatment and/or Cure of HIV Infection (if Bone Marrow Transplant Recipient is either HIV Positive or at High Risk of HIV Infection); Susceptibility to Disease Processes associated with HIV Infection (including but not limited to HIV-associated Dementia and/or Kaposi Sarcoma); Risk of Mother-to-child HIV Transmission Susceptibility; or HIV-Associated Focal Segmental Glomerulosclerosis, determined. An HIV Panel can determine the risk or predisposition of a subset of the aforementioned phenotypes, such as at least 1, 2, or 3 of the following phenotypes: Human Immunodeficiency Virus (HIV) Infection Susceptibility to or Resistance against; Rate of Progression and/or Prognosis with HIV Infection; or HIV Medication Metabolism and/or Hypersensitivity and/or Dose and/or Choice of Medication used for Treatment or Prophylaxis.

An individual can also be tested with the Viral Hepatitis Panel. The individual may have an abnormal liver function test or a blood test indicative of hepatitis infection. An individual interested in being tested with the Viral Hepatitis Panel may have a history of travel to hepatitis endemic area; history of unprotected sex, history of nasal drug (such as narcotics, cocaine, methamphetamine, ketamine) usage, history of blood transfusion, history of needle stick, direct contact with someone else's blood, or any combination thereof. An individual interested in being tested with the Viral Hepatitis Panel may have one or more of the following symptoms: jaundice, pruritus, pain, or ascites. Individuals with or without a current or prior diagnosis of viral hepatitis may also be interested in other genetic links to phenotypes, and their risk or predisposition to those phenotypes, that are related to viral hepatitis. They can also have a personal or family history of hepatitis, such as viral hepatitis infection. An individual can be tested for their risk or predisposition of an individual for phenotypes, such as at least 1, 2, 3, 4, 5, or 6 of the following phenotypes: Viral Hepatitis Susceptibility; Effectiveness and/or Response and/or Adverse Effects and/or Sensitivity to Medications Used to Treat Viral Hepatitis Infections; Rate and/or Likelihood of Viral Hepatitis Clearance; Severity of Liver Disease with Viral Hepatitis Infection; Risk of Viral Hepatitis Recurrence after Liver Transplantation; or Modifier of Vaccine-induced Immunity to Viral Hepatitis Infection. A Viral Hepatitis Panel can determine the risk or predisposition of a subset of the aforementioned phenotypes, such as at least 1, 2, 3, or 4 of the following phenotypes: Viral Hepatitis Susceptibility; Effectiveness and/or Response and/or Adverse Effects and/or Sensitivity to Medications Used to Treat Viral Hepatitis Infections; Rate and/or Likelihood of Viral Hepatitis Clearance; or Severity of Liver Disease with Viral Hepatitis Infection.

An individual can also be tested with the Malaria Panel. The individual may have a blood test suggestive or indicative of malarial infection, antigen detection test suggestive or indicative of malarial infection, parasitemia, anemia; splenomegaly and/or hepatomegaly, abnormal renal function test, or any combination thereof. Individuals with or without a current or prior diagnosis of malaria may also be interested in other genetic links to phenotypes, and their risk or predisposition to those phenotypes, that are related to malaria. An individual tested with the Malaria Panel may live in or have a history of travel to malaria endemic areas. An individual tested with the Malaria Panel may have one or more of the following symptoms: fever, fatigue, shivering, arthralgia, vomiting, convulsions, headache, or abnormal posturing. The individual tested with the Malaria Panel may also have a personal or family history of malaria or HIV infection. The individual can have the risk or predisposition of all the conditions listed in herein.

Individuals may select the Malaria Panel, which can be used to determine the risk or predisposition of an individual for phenotypes, such as at least 1, 2, 3, or 4 of the following phenotypes: Malaria Susceptibility; Metabolism and/or Dose and/or Choice and/or Sensitivity and/or Adverse Reaction to Anti-Malaria Medication and/or Malaria Prophylaxis; Prognosis and/or Severity and/or Symptomatology and/or Mortality with Malarial Infection; Glucose-6-phosphate Dehydrogenase Deficiency; or Iron Deficiency and/or Iron Deficiency Anemia during Malaria Season, determined. A Malaria Panel can determine the risk or predisposition of a subset of the aforementioned phenotypes, such as at least 1, 2, 3, or 4 of the following phenotypes: Malaria Susceptibility; Metabolism and/or Dose and/or Choice and/or Sensitivity and/or Adverse Reaction to Anti-Malaria Medication and/or Malaria Prophylaxis; Prognosis and/or Severity and/or Symptomatology and/or Mortality with Malarial Infection; or Glucose-6-phosphate Dehydrogenase Deficiency.

Individuals suffering from a terminal disease or disorder, suffering from acute or chronic pain, or who are under hospice care or palliative care may be tested with a palliative care panel, see, e.g., the Palliative Care Panel or with a pain panel, see, e.g., the Pain Panel. An individual with a personal medical history of a chronic disease or disorder or a personal or family medial history of diminished or augmented response to pain medication may also be tested with a Pain Panel. An individual with a personal or family medical history of certain mental health issues (e.g., depression, suicidality, etc.) may be tested with a pain panel In some cases, an individual with symptoms suggestive of pain (e.g., an abnormal heart rate or abnormal blood pressure or facial expression(s) or posturing indicative of pain) may also be tested with a Palliative Care panel and/or a Pain Panel. An individual with a progressive, debilitating, degenerative, or fatal disease (such as cancer, amyotrophic lateral sclerosis, heart failure such as end-stage heart failure, end-stage liver disease, or end-stage lung disease) may be tested with a Palliative Care Panel. This panel may be ordered by a healthcare provider such as a Palliative Care Specialist, a psychiatrist, a therapist, a hospice nurse, a hospice center, or other end-of-life care provider.

Decisions about an individual's care or treatment may take into account results from a Pain Panel or Palliative Care Panel. An individual diagnosed with a terminal disease or disorder and/or his or her caretakers (e.g., medical professionals, guardians, hospice workers, physicians, registered nurses, nursing assistants, social workers, hospice chaplains, physiotherapists, occupational therapists, complementary therapists, volunteers, family members, friends, psychiatrists, psychologists, or home health care aides) may consider such results when making decisions about how best to provide care, comfort and support for the individual during his or her illness. For example, if the individual is found to be at risk for stress to cause depressive symptoms or more vulnerable to stressful situations such as a chronic or terminal disease, the individual or individual's caretaker(s) may more aggressively monitor the individual's mental health and may also be more quick to offer mental health services, more intensive palliative care interactions or services, or more insightful palliative care interactions or services, or to provide a better social support network, to such individual, that may help decrease, relieve, treat, mitigate, come to terms with, better understand or control the individual's depression, anxiety, fear, anger, psychospiritual distress, and/or existential distress. In addition, a positive result for the condition of stress causing anxiety may be accompanied by a reflex test for other conditions such as the likelihood that a medication such as a serotonin reuptake inhibitor (e.g., fluoxetine, fluoxetine hydrochloride, Prozac, dapoxetine, citalopram, escitalopram, sertraline, fluvoxamine, paroxetine, zimelidine, etc.) will successfully treat the symptom of increased anxiety, or for determining a genetically tailored dose for a medication such as a serotonin reuptake inhibitor.

Risks for all of the phenotypes, such as conditions in the panels, or a subset of the phenotypes, such as conditions, may be determined instead. In some cases, a Pain Panel may be used to test an individual for a set of two or more risks (or predisposition), for example, such set may include one of the following sets of risks: his or her disposition for pain tolerance and his or her disposition to metabolize pain medication; his or her disposition for pain tolerance and his or her disposition to react to main medication (including, adverse reactions; sensitivities; metabolism); and his or her disposition for pain tolerance and his or her disposition for back pain. In some cases, a Pain Panel may comprise at least an individual's predisposition for pain tolerance. Individuals may select the Pain Panel, which can be used to determine the risk or predisposition of an individual for phenotypes, such as at least 1, 2, 3, 4, 5, or 6 of the following phenotypes: Pain Tolerance; Analgesic Effectiveness and/or Sensitivity to Pain Medicine and/or Dosage of Pain Medicine Required for Analgesic Effect; Depression and/or Seasonal Affective Disorder; Fibromyalgia; Stressful Life Events causing Depressive Symptoms and/or Diagnosable Depression and/or Suicidality and/or Anxiety (including but not limited to Mental Vulnerability to Stress and/or Disease); or Suicidality. A Pain Panel can determine the risk or predisposition of a subset of the aforementioned phenotypes, such as at least 1, 2, 3, or 4 of the following phenotypes: Pain Tolerance; Analgesic Effectiveness and/or Sensitivity to Pain Medicine and/or Dosage of Pain Medicine Required for Analgesic Effect; Depression and/or Seasonal Affective Disorder; or Fibromyalgia.

In some cases, a Palliative Care Panel may be used to test an individual for a set of two or more risks (or predisposition), for example, such set may include one of the following sets of risks: risk of suicidality and disposition for pain tolerance; predisposition for stress to cause depression/anxiety and predisposition for pain tolerance; predisposition for a negative internal affective state in response to pain and risk of suicidality; predisposition for lack of social support to cause depressive symptoms and predisposition for stress to cause depressive symptoms; predisposition for pain tolerance and predisposition for lack of social support to cause depressive symptoms. In some cases, a Palliative Care Panel may include at least the condition of disposition for pain tolerance. In some cases, a Palliative Care Panel may include at least the condition of negative internal affective state in response to pain. The Palliative Care Panel can determine the risk or predisposition of an individual for phenotypes, such as at least 1, 2, 3, 4, 5, 6, 7, 8, or 9 of the following phenotypes: Suicidality; Negative Internal Affective State in Response to Pain; Pain Tolerance; Analgesic Effectiveness and/or Sensitivity to Pain Medicine and/or Dosage of Pain Medicine Required for Analgesic Effect; Stressful Life Events causing Depressive Symptoms and/or Diagnosable Depression and/or Suicidality and/or Anxiety (including but not limited to Mental Vulnerability to Stress and/or Disease); Opioid-induced Respiratory Depression; Thrombophilia and/or Thromboembolic Disease; Personality Traits (Including but not Limited to Handling of Stress, Degree of Extroversion and/or Introversion, Openness, Degree of Altruism, Level of Aggression, Oppositional Behaviors, Violent Delinquency, Serious Delinquency, Coping Style, Type A Behavior, Way in Which Anger is Expressed, Novelty Seeking Behavior, and/or Harm Avoidance); or DNA Banking (To Save for Future Analysis). A Palliative Care Panel can determine the risk or predisposition of a subset of the aforementioned phenotypes, such as at least 1, 2, 3, 4, or 5 of the following phenotypes: Suicidality; Negative Internal Affective State in Response to Pain; Pain Tolerance; Analgesic Effectiveness and/or Sensitivity to Pain Medicine and/or Dosage of Pain Medicine Required for Analgesic Effect; or Stressful Life Events causing Depressive Symptoms and/or Diagnosable Depression and/or Suicidality and/or Anxiety (including but not limited to Mental Vulnerability to Stress and/or Disease).

Individuals interested in being tested for their risk or predisposition to conditions affecting the loco-motor system including joints, muscles, connective tissues, soft tissues around the joints and bones, autoimmune conditions, immunological conditions, inflammatory conditions or other condition related to the field of rheumatology can use the Rheumatology Panel Alpha and/or Beta. Individuals may have an abnormal Anti-Nuclear Antibodies (ANA) level, abnormal bone mineral density, radiologic exam showing degenerative joint disease, abnormal Erythrocyte Sedimentation Rate (ESR) level, abnormal C-reactive protein (CRP) level, or a combination thereof. The individuals may be approximately 35 years or older, have an occupation associated with repetitive movements, joint stress and/or bone stress, may be a nonprofessional or professional athlete, or a combination thereof. An individual interested in selecting the Rheumatology Panel may be experiencing one or more of the following symptoms: joint pain, joint swelling, enlargement of joints, or decreased range of motion of joints. The individual may have a personal or family history of arthritis (including but not limited to osteoarthritis and/or inflammatory polyarthritis and/or juvenile idiopathic arthritis), osteoporosis, fibromyalgia, autoimmune rheumatologic disease (including but not limited to rheumatoid arthritis, systemic lupus erythematosus, systemic sclerosis, Sjögren's syndrome, polymyositis, etc.), or any combination thereof.

Individuals may select the Rheumatology Panel Alpha, which can be used to determine the risk or predisposition of all the phenotypes, or a subset, such as at least 1, 2, 3, 4, 5, 6, or 7 of the following phenotypes: Osteoporosis and/or Osteoporotic Fracture; Lumber Disc Disease; Osteoarthritis; Fibromyalgia; Rheumatoid Arthritis; Systemic Lupus Erythematosus (SLE); or Ankylosing Spondylitis. A Rheumatology Panel Alpha can determine the risk or predisposition of a subset of the aforementioned phenotypes, such as at least 1, 2, 3, or 4 of the following phenotypes: Osteoporosis and/or Osteoporotic Fracture; Lumber Disc Disease; Osteoarthritis; or Fibromyalgia.

Individuals may select the Rheumatology Panel Beta, which can be used to determine the risk or predisposition of an individual for phenotypes, such as at least 1, 2, 3, 4, 5, 6, 7, 8, or 9 of the following phenotypes: Rheumatoid Arthritis; Systemic Lupus Erythematosus (SLE); Ankylosing Spondylitis; Inflammatory Polyarthritis; Systemic Sclerosis; Myositis; Psoriatic Arthritis; Fibromyalgia; or Sjögren's Syndrome. A Rheumatology Panel Beta can determine the risk or predisposition of a subset of the aforementioned phenotypes, such as at least 1, 2, 3, 4, 5, or 6 of the following phenotypes: Rheumatoid Arthritis; Systemic Lupus Erythematosus (SLE); Ankylosing Spondylitis; Inflammatory Polyarthritis; Systemic Sclerosis; or Myositis.

Individuals with a high risk of systemic lupus erythematosus (SLE) or rheumatoid arthritis (RA), such as determined by the Rheumatology Panel, may be interested in being tested with the SLE or RA Panels, or both. Alternatively, individuals not tested with the Rheumatology Panel may be interested in the SLE, RA, or both panels. An individual with abnormal antinuclear antibody, abnormal anti-extractable nuclear antigen level, positive for anti-Smith antibody, positive for anti-ds DNA antibody, positive for antiphospholipid antibody, false positive in a serological test for syphilis, or any combination thereof, may select the SLE Panel for determining their risk or predisposition of an individual for phenotypes, such as at least 1, 2, 3, or 4 of the following phenotypes: Systemic Lupus Erythematosus (SLE); Prognosis and/or Severity of SLE; Symptomatology with SLE (including but not limited to Rash, Oral Ulcers, Serositis, Nephritis, and/or Autoantibodies); or Age of Disease Onset of SLE. A Systemic Lupus Erythematosus Panel can determine the risk or predisposition of a subset of the aforementioned phenotypes, such as at least 1, 2, or 3 of the following phenotypes: Systemic Lupus Erythematosus (SLE); Prognosis and/or Severity of SLE; or Symptomatology with SLE (including but not limited to Rash, Oral Ulcers, Serositis, Nephritis, and/or Autoantibodies), determined. Individuals with or without a current or prior diagnosis of SLE may also be interested in other genetic links to phenotypes, and their risk or predisposition to those phenotypes, that are related to SLE, and may also be interested in the SLE Panel, for example.

Individuals interested in being tested with the Rheumatoid Arthritis (RA) Panel may have an abnormal radiologic exam of joints, abnormal immunological blood test (including, but not limited to, rheumatoid factor and/or anti-citrullinated protein antibodies), abnormal anti-nuclear antibody or any combination thereof. The individual may be a current or former tobacco smoker, have passive exposure to tobacco smoke, or both. The individual may have symptoms such as joint pain, stiffness, swelling, subcutaneous nodules, or any combination thereof. The individual's medical history may include a herpes infection, such as a human herpes virus 6 infection. Their personal and family medical history may include rheumatoid arthritis, autoimmune disease, Epstein-Barr virus infection, or any combination thereof. Individuals with or without a current or prior diagnosis of RA may also be interested in other genetic links to phenotypes, and their risk or predisposition to those phenotypes, that are related to RA. The individual may have their risk (as stated, the term risk may refer to one or more of the following: increased or decreased risk of multifactorial phenotype(s) or carrier status of monogenic or polygenic phenotype(s), such as non-carrier, carrier but not affected, affected, or likely affected) or predisposition of an individual for phenotypes, such as at least 1, 2, 3, 4, 5, or 6 of the following phenotypes: Rheumatoid Arthritis; Effectiveness and/or Dose and/or Choice and/or Adverse Reaction to Medications used to Treat Rheumatoid Arthritis; Prognosis and/or Disease Severity and/or Functional Outcome with Rheumatoid Arthritis; Effect of Cigarette Smoking Exposure upon Rheumatoid Arthritis; Hypertension with Rheumatoid Arthritis; or Chronic Iridocyclitis with Rheumatoid Arthritis, determined. A Rheumatoid Arthritis Panel can determine the risk or predisposition of a subset of the aforementioned phenotypes, such as at least 1, 2, or 3 of the following phenotypes: Rheumatoid Arthritis; Effectiveness and/or Dose and/or Choice and/or Adverse Reaction to Medications used to Treat Rheumatoid Arthritis; or Prognosis and/or Disease Severity and/or Functional Outcome with Rheumatoid Arthritis.

Individuals interested in being tested for their risk or predisposition to another inflammatory or rheumatological phenotypes, such as conditions, may be interested in the Gout Panel. The Gout Panel can determine the risk or predisposition of an individual for phenotypes, such as at least 1, 2, or 3 of the following phenotypes: Gout; Effectiveness and/or Choice and/or Dose and/or Adverse Reaction to Medications Used to Treat and/or Prevent Gout; Allopurinol-induced Severe Cutaneous Adverse Reactions (SCAR); or Metabolism of and/or Response to and/or Effectiveness of and/or Adverse Reactions and/or Dosing and/or Choice of Opiates Required for Analgesic Effect. A Gout Panel can determine the risk or predisposition of a subset of the aforementioned phenotypes, such as at least 1, 2, or 3 of the following phenotypes: Gout; Effectiveness and/or Choice and/or Dose and/or Adverse Reaction to Medications Used to Treat and/or Prevent Gout; or Allopurinol-induced Severe Cutaneous Adverse Reactions (SCAR). Individuals with or without a current or prior diagnosis of gout may also be interested in other genetic links to phenotypes, and their risk or predisposition to those phenotypes, that are related to gout.

Individuals may also be interested in their risk or predisposition to auditory or opthalmological phenotypes, such as conditions, and may therefore be tested with an Auditory Panel or Opthalmology Panel. For example, an individual with an abnormal auditory test result (such as, but not limited to, behavioral audiogram and/or electro-physiological testing), occupational exposure to loud and/or harmful noise, a history of listening to loud music, experiencing tinnitus, decreased hearing, or any combination thereof, may be interested in selecting the Auditory Panel. The individual may have a personal or family history of hearing impairment and/or deafness. The individual may choose to have the risk or predisposition of an individual for phenotypes, such as at least 1, 2, 3, 4, or 5 of the following phenotypes: Hearing Impairment (Including Deafness and/or Hearing Loss); Age-Related Hearing Impairment and/or Hearing Loss; Noise-induced Hearing Impairment and/or Hearing Loss; Tinnitus; Meniere Disease and/or Balance Abnormalities; or Otitis. An Auditory Panel can determine the risk or predisposition of a subset of the aforementioned phenotypes, such as at least 1, 2, 3, or 4 of the following phenotypes: Hearing Impairment (Including Deafness and/or Hearing Loss); Age-Related Hearing Impairment and/or Hearing Loss; Noise-induced Hearing Impairment and/or Hearing Loss; or Tinnitus.

Individuals interested in the Opthalmology Panel may include, but not be limited to, individuals with an abnormal ophthalmologic exam. Individuals may also be current or former tobacco smokers. The individuals interested in the Opthalmology Panel may also have one or more of the following symptoms: decreased visual acuity, blindness, blurry vision, eye pain, sensitivity to light, or soreness. The individual may have a medical history comprising one or more of the following: visual impairment, blindness, cataract, Diabetes Mellitus (Type I or Type II), diabetic retinopathy, hypertension, glaucoma, macular degeneration, or being overweight or obese. The individual may have a family history of cataract, visual impairment, blindness, glaucoma, macular degeneration, uveitis, corneal opacity, tachoma, or any combination thereof. An individual can have the risk or predisposition of an individual for phenotypes, such as at least 1, 2, 3, 4, 5, 6, 7, 8, 9, 10, 11, or 12 of the following phenotypes: Macular Degeneration; Glaucoma; Cataract; Myopia; Hyperopia; Night Blindness; Color Blindness & Achromatopsia; Leber Congenital Amaurosis; Diabetic Retinopathy; Sjögren's Syndrome; Variation in Color Perception; or Dry Eye Syndrome. An Opthalmology Panel can determine the risk or predisposition of a subset of the aforementioned phenotypes, such as at least 1, 2, 3, 4, or 5 of the following phenotypes: Macular Degeneration; Glaucoma; Cataract; Myopia; or Hyperopia.

Individuals may also be interested in their risk or predisposition to kidney conditions and urological conditions and be tested with the Urology and Nephrology Panel. The individual may have an abnormal prostate specific antigen (PSA) level(s), abnormal early prostate cancer antigen-2 (EPCA-2) level(s), abnormal sarcosine urine level(s), hematuria, abnormal prostate physical exam, be a current or former tobacco smoker, be of approximately 35, 40, 45, or 50 years of age or older, or any combination thereof. The individual may have one or more of the following symptoms: erectile dysfunction or urinary abnormalities (including, but not limited to, incontinence, frequency, hesitancy, and/or urgency). The individual interested in their risk or predisposition to kidney conditions and urological conditions may have a personal or family history of one or more of the following: prostate cancer; erectile dysfunction, heart disease, atherosclerosis, diabetes mellitus (type I or type II), nephrolithiasis, urolithiasis, renal cancer, bladder cancer, fertility issues, polycystic kidney disease, or IgA nephropathy. The individual can choose to have the risk or predisposition of an individual for phenotypes, such as at least 1, 2, 3, 4, 5, 6, 7, 8, or 9 of the following phenotypes: Male Fertility/Infertility (including but not limited to Abnormal Sperm Count and/or Abnormal Sperm Motility and/or Abnormal Sperm Morphology); Erectile Dysfunction Medication Treatment Effectiveness and/or Sensitivity; Prostate Cancer; Nephrolithiasis and/or Urolithiasis; Bladder Cancer and/or Kidney Cancer and/or Adrenal Cancer; IgA Nephropathy; Diabetic Nephropathy; Polycystic Kidney Disease; or Risk of Complications with Hemodialysis, determined. An Urology & Nephrology Panel can determine the risk or predisposition of a subset of the aforementioned phenotypes, such as at least 1, 2, or 3 of the following phenotypes: Male Fertility/Infertility (including but not limited to Abnormal Sperm Count and/or Abnormal Sperm Motility and/or Abnormal Sperm Morphology); Erectile Dysfunction Medication Treatment Effectiveness and/or Sensitivity; or Prostate Cancer.

Individuals interested in the genetic basis of their pulmonary disease, sleep health or restlessness, or perhaps their children's, to see the link to genetics or if to another possible cause, may be interested in the Pulmonology Panel and Sleep Medicine Panel for themselves or their children. Individuals interested in these panels may also be interested in the Asthma Panel, Chronic Obstructive Pulmonary Disease Panel; Pulmonary Hypertension Panel, or any combination thereof.

The individual to be tested, adult or child, with the Pulmonology Panel may have an abnormal pulmonary function test and/or abnormal chest radiologic exam. They may be a current or former tobacco smoker, experience dyspnea, coughing, clubbing, hemoptysis, or any combination thereof. They may have a personal or family history of one or more of the following conditions: lung cancer, emphysema, chronic bronchitis; chronic obstructive pulmonary disease, asthma, chronic cough, lung disease, pulmonary hypertension, or alpha-1-antitrypsin deficiency. Risks for all of the conditions in the panel, or a subset of the conditions may be determined instead. For example, all the risk or predisposition of an individual for phenotypes, such as at least 1, 2, 3, 4, 5, 6, or 7 of the following phenotypes: Lung Cancer; Nicotine Addiction and/or Nicotine Dependence; Asthma; Chronic Obstructive Pulmonary Disease (COPD); Pulmonary Hypertension; Alpha-1 Antitrypsin Deficiency; or Cystic Fibrosis, can be determined. A Pulmonology Panel can determine the risk or predisposition of a subset of the aforementioned phenotypes, such as at least 1, 2, 3, or 4 of the following phenotypes: Lung Cancer; Nicotine Addiction and/or Nicotine Dependence; Asthma; or Chronic Obstructive Pulmonary Disease (COPD).

In addition to the Pulmonology Panel, an individual can be tested, concurrent, subsequent, or prior to testing with the Pulmonology Panel, with the Asthma Panel; Chronic Obstructive Pulmonary Disease Panel; Pulmonary Hypertension Panel, or any combination thereof. For example, and individual may have a high risk of COPD as determined by testing with the Pulmonology Panel and decide to be tested with the COPD Panel. Alternatively, an individual may choose any of the panels, such as the Asthma Panel; Chronic Obstructive Pulmonary Disease Panel; Pulmonary Hypertension Panel, or any combination thereof, without testing with the Pulmonology Panel.

For example, an individual with an abnormal peak flow rate, abnormal pulmonary function test, abnormal capnography, abnormal pulse oximetry, or any combination thereof, may be tested with the Asthma Panel. The individual may be living in an area with poor environmental air quality, may be a current or former tobacco smoker, have passive exposure to tobacco smoke, was birthed by mother having cesarean section, had antibiotic use early in life, experience psychological stress, or any combination thereof. The individual may have difficulty breathing, dyspnea, wheezing; and/or atopy. The individual may also have a personal or family history of asthma, allergy, atopy, smokers. The individual being tested may have the risk or predisposition of an individual for phenotypes, such as at least 1, 2, 3, 4, or 5 of the following phenotypes: Asthma; Aspirin-induced Asthma; Asthma Exacerbations from Exposure to Dust and/or Endotoxins and/or Cockroaches; Response to and/or Effectiveness and/or Adverse Effects of Medications used to Treat and/or Prevent Asthma and/or Asthma Attacks; or Prognosis and/or Severity and/or Lung Function with Asthma.

Individuals being tested with the COPD Panel may have an abnormal pulmonary function test, diminished breath sounds, a chest radiologic exam suggestive or indicative of COPD, of any combination thereof. The individual may be a current or former tobacco smoker, have passive exposure to tobacco smoke, have occupational exposure to workplace dust, cotton, chemicals and/or fumes; be exposed to urban air pollution, or any combination thereof. The individual may be having symptoms of dyspnea, coughing, wheezing, and/or tachypnea and may have a personal or family history of chronic obstructive pulmonary disease, chronic bronchitis, and or emphysema. Individuals with or without a current or prior diagnosis of COPD may also be interested in other genetic links to phenotypes, and their risk or predisposition to those phenotypes, that are related to COPD. An individual may select to have the risk or predisposition of an individual for phenotypes, such as at least 1, 2, 3, 4, or 5 of the following phenotypes: Chronic Obstructive Pulmonary Disease (COPD); Response to and/or Effectiveness and/or Adverse Effects of Medications used to Treat and/or Prevent COPD; Prognosis and/or Survival and/or Rate of Decline of Lung Function with COPD; Degree of Pulmonary Hypertension with COPD; or Nicotine Addiction and/or Nicotine Dependence, to be determined. A Chronic Obstructive Pulmonary Disease Panel can determine the risk or predisposition of a subset of the aforementioned phenotypes, such as at least 1, 2, 3, or 4 of the following phenotypes: Chronic Obstructive Pulmonary Disease (COPD); Response to and/or Effectiveness and/or Adverse Effects of Medications used to Treat and/or Prevent COPD; Prognosis and/or Survival and/or Rate of Decline of Lung Function with COPD; or Degree of Pulmonary Hypertension with COPD.

Individuals being tested with the Pulmonary Hypertension Panel may have one or more of the following: abnormal pulmonary function test, altered heart sounds on cardiac physical exam, elevated jugular venous pressure, ascites, hepatojugular reflux, abnormal pulmonary artery occlusion pressure, or abnormal pulmonary vascular resistance. An individual may be a current or former tobacco smoker, have a history of cocaine use, methamphetamine use and/or alcohol use. An individual may have one or more of the following symptoms: dyspnea, fatigue, cough, chest pain, clubbing, peripheral edema, or syncope. Individuals with or without a current or prior diagnosis of pulmonary hypertension may also be interested in other genetic links to phenotypes, and their risk or predisposition to those phenotypes, that are related to pulmonary hypertension. An individual may have a personal or family history of pulmonary hypertension, emphysema, chronic obstructive pulmonary disease, and/or cirrhosis. The individual may choose to have the risk or predisposition of an individual for phenotypes, such as at least 1, 2, 3, or 4 of the following phenotypes: Pulmonary Hypertension; Prognosis and/or Severity of Pulmonary Hypertension; Age of Onset of Pulmonary Hypertension; or Prognosis and/or Survival and/or Allograft Fibrosis in Lung Transplant Recipients, be determined. Individuals interested in their risk or predisposition to conditions related to sleep may be interested in the Sleep Medicine Panel. The individual may have an abnormal sleep study exam, fatigue, lethargy, insomnia, hypersomnia, and/or difficulty concentrating. The individual may also have a personal history of being overweight or obese and/or snoring, and may also have a personal or family history of sleep apnea, insomnia, narcolepsy, and/or idiopathic hypersomnia. An individual may choose to have the risk or predisposition of an individual for phenotypes, such as at least 1, 2, 3, 4, or 5 of the following phenotypes: Sleep Apnea; Narcolepsy; Idiopathic Hypersomnia; Effect of Stimulant(s) on Cognition; Restless Leg Syndrome and/or Periodic Limb Movements in Sleep; Insomnia and/or Level of Sleepiness; Number of Awakenings During Sleep and/or Intensity Level of Sleep, to be determined. A Sleep Medicine Panel can determine the risk or predisposition of a subset of the aforementioned phenotypes, such as at least 1, 2, 3, 4, or of the following phenotypes: Sleep Apnea; Narcolepsy; Idiopathic Hypersomnia; Effect of Stimulant(s) on Cognition; or Restless Leg Syndrome and/or Periodic Limb Movements in Sleep; Insomnia and/or Level of Sleepiness.

Individuals interested in their risk or predisposition to cancer may be tested with the Oncology Panel. Individuals with a radiologic exam, biopsy, or blood test suspicious for or indicative of a precancerous state or cancer can be tested with the Oncology Panel. An individual with a physical exam suspicious for cancer can also be tested. Individuals that are current or former tobacco smokers, or have alcohol abuse or alcohol dependence may also be interested in being tested. The individual may be experiencing weight loss and/or fatigue, and have a personal or family history of cancer (including, but not limited to, lung, breast, ovarian, colorectal, skin, prostate, testicular, gastric, liver, pancreatic, brain, nerve, adrenal, blood, bone, leukemia, lymphoma, esophageal, nasopharyngeal, connective tissue, soft tissue, cervical, or a cancer syndrome). Individuals with or without a current or prior diagnosis of cancer may also be interested in other genetic links to phenotypes, and their risk or predisposition to those phenotypes, that are related to cancer. The individual may be tested for their risk or predisposition of an individual for phenotypes, such as at least 1, 2, 3, 4, 5, 6, 7, 8, or 9 of the following phenotypes: Lung Cancer; Colorectal Cancer; Breast Cancer and/or Ovarian Cancer; Prostate Cancer; Melanoma; Gastric Cancer; Leukemia; Lymphoma; or Pancreatic Cancer. An Oncology Panel can determine the risk or predisposition of a subset of the aforementioned phenotypes, such as at least 1, 2, 3, 4, 5, or 6 of the following phenotypes: Lung Cancer; Colorectal Cancer; Breast Cancer and/or Ovarian Cancer; Prostate Cancer; Melanoma; or Gastric Cancer.

Individuals can also be tested for genetic variants provided in the panels described herein for specific types of cancers, such as the Breast Cancer Panel, Ovarian Cancer Panel, Lung Cancer Panel, Colorectal Cancer Panel, Prostate Cancer Panel, Skin Cancer Panel, Leukemia Panel, Lymphoma Panel, Gastric and Gastrointestinal Cancer Panel, Head & Neck Cancer Panel, Multiple Myeloma Panel or any combination thereof, concurrent with, prior to, or subsequent to testing or analysis with the Oncology panel. For example, an individual may first have the results of an Oncology Panel test analyzed and then decide to have a Lung Cancer test because the initial Oncology Panel results showed the individual having a high risk of lung cancer. Alternatively, an individual may not be tested with the Oncology Panel and be tested with one or more of the specific cancer type panels. As with all of the panels, these panels can be run on any genetic material from an embryo or fetus, including but not limited to cells from an amniocentesis or chorionic villus sampling (CVS) or from embryo or fetal genetic material obtained through non-invasive prenatal test methods, such as embryonic or fetal cells derived from maternal/fetal cell sorting, or embryonic or fetal genetic material derived from any other method, including fetal oligonucleotides, fetal nucleic acid(s), fetal DNA, fetal cells, or any other fetal genetic material that can be isolated from the developing fetus, the amnion, the amniotic sac, the blood of a pregnant female or via peripheral or central blood draw(s) from the pregnant female.

An individual tested for genetic variants provided in the Breast Cancer Panel may have their risk or predisposition analyzed for all the phenotypes, or a subset, such as at least 1, 2, 3, 4, 5, 6, or 7 of the following phenotypes: Breast Cancer; Tamoxifen Effectiveness, Sensitivity, and/or Adverse Reaction; Prognosis with Breast Cancer (including but not limited to Survival and/or Mortality); Effectiveness and/or Metabolism and/or Choice and/or Dose and/or Adverse Reaction of Medications used to Treat Breast Cancer; Radiosusceptibility and/or Residual DNA Damage Level to Radiation; Chemotherapy-induced Leukemia; or Thrombophilia and/or Thromboembolic Disease. A Breast Cancer Panel can determine the risk or predisposition of a subset of the aforementioned phenotypes, such as at least 1, 2, or 3 of the following phenotypes: Breast Cancer; Tamoxifen Effectiveness, Sensitivity, and/or Adverse Reaction; or Prognosis with Breast Cancer (including but not limited to Survival and/or Mortality). The individual may be under approximately 65 years of age, have an increase in breast mass, loss in weight, or any combination thereof. Individuals with or without a current or prior diagnosis of breast cancer may also be interested in other genetic links to phenotypes, and their risk or predisposition to those phenotypes, that are related to breast cancer. The individual may have a personal or family history of breast cancer and/or ovarian cancer, or a family medical history of unknown cancer.

An individual tested for genetic variants provided in the Ovarian Cancer Panel may have their risk or predisposition analyzed for all the phenotypes, or a subset, such as at least 1, 2, 3, 4, 5, or 6 of the following phenotypes: Ovarian Cancer; Effectiveness and/or Metabolism and/or Choice and/or Dose and/or Adverse Reaction of Medications used to Treat Ovarian Cancer; Prognosis with Ovarian Cancer (including but not limited to Survival and/or Mortality); Chemotherapy-induced Leukemia; Radiosusceptibility and/or Residual DNA Damage Level to Radiation; or Thrombophilia and/or Thromboembolic Disease, determined. An Ovarian Cancer Panel can determine the risk or predisposition of a subset of the aforementioned phenotypes, such as at least 1, 2, or 3 of the following phenotypes. Ovarian Cancer; Effectiveness and/or Metabolism and/or Choice and/or Dose and/or Adverse Reaction of Medications used to Treat Ovarian Cancer; or Prognosis with Ovarian Cancer (including but not limited to Survival and/or Mortality). The individual may have an abnormal pelvic radiologic exam (for example, MRI, ultrasound, or CT scan), and/or abnormal ovarian biopsy showing precancerous or cancerous cells. The individual may be under approximately 65 years of age, have an increase in pelvic mass, loss in weight, or any combination thereof. Individuals with or without a current or prior diagnosis of ovarian cancer may also be interested in other genetic links to phenotypes, and their risk or predisposition to those phenotypes, that are related to ovarian cancer. The individual may have a personal or family history of breast cancer and/or ovarian cancer, or a family medical history of unknown cancer.

An individual tested for genetic variants provided in the Lung Cancer Panel, may have their risk or predisposition analyzed for all the phenotypes, or a subset, such as at least 1, 2, 3, 4, or 5 of the following phenotypes: Lung Cancer; Effectiveness and/or Metabolism and/or Choice and/or Dose and/or Adverse Reaction of Medication used to Treat Lung Cancer; Prognosis with Lung Cancer (including but not limited to Survival and/or Mortality); Radiosusceptibility and/or Residual DNA Damage Level to Radiation; or Thrombophilia and/or Thromboembolic Disease, be determined. A Lung Cancer Panel can determine the risk or predisposition of a subset of the aforementioned phenotypes, such as at least 1, 2, or 3 of the following phenotypes: Lung Cancer; Effectiveness and/or Metabolism and/or Choice and/or Dose and/or Adverse Reaction of Medication used to Treat Lung Cancer; or Prognosis with Lung Cancer (including but not limited to Survival and/or Mortality). The individual may have an abnormal chest radiologic exam (for example, MRI, ultrasound, or CT scan), and/or abnormal lung biopsy showing precancerous or cancerous cells. The individual may be a current or former tobacco smoker, experience passive exposure to tobacco smoke, experience environmental and/or occupational exposure to substances carcinogenic to the lung, or any combination thereof. Individuals with or without a current or prior diagnosis of lung cancer may also be interested in other genetic links to phenotypes, and their risk or predisposition to those phenotypes, that are related to lung cancer. The individual may have one or more of the following symptoms: loss in weight, coughing, hemoptysis, dyspnea, dysphonia; bone pain, or fever. The individual may have a personal or family history of lung cancer, viral infection (including such as, but not limited to, human papillomavirus, John Cunningham virus (JC virus), simian virus 40, BK virus, and cytomegalovirus), Lambert-Eaton myasthenic syndrome, or of being around or exposed to tobacco smoke or asbestos.

An individual tested for genetic variants provided in the Colorectal Cancer Panel may have their risk or predisposition analyzed for all the phenotypes, or a subset, such as at least 1, 2, 3, 4, 5, 6, or 7 of the following phenotypes: Colorectal Cancer; Prognosis with Colorectal Cancer (including but not limited to Survival and/or Mortality); Toxicity and/or Effectiveness and/or Dose and/or Choice of Chemotherapeutic Medications to Treat Colorectal Cancer; Chemotherapy-Induced Leukemia; Thrombophilia and/or Thromboembolic Disease; Association of Colorectal Cancer with Consumption of Specific Food (including but not limited to Dietary Red Meat); or Colorectal Cancer with Exposure to Tobacco Smoke, be determined. A Colorectal Cancer Panel can determine the risk or predisposition of a subset of the aforementioned phenotypes, such as at least 1, 2, or 3 of the following phenotypes: Colorectal Cancer; Prognosis with Colorectal Cancer (including but not limited to Survival and/or Mortality); or Toxicity and/or Effectiveness and/or Dose and/or Choice of Chemotherapeutic Medications to Treat Colorectal Cancer. The individual being tested may have one or more of the following: anemia, abnormal colonoscopy, abnormal sigmoidoscopy, abnormal virtual colonoscopy, abnormal capsule endoscopy, or abnormal colon biopsy suggestive or indicative of precancerous and/or cancerous cells; abnormal fecal occult blood testing, abnormal stool DNA test indicative of colorectal cancer or possible colorectal cancer, bowel obstruction, or low selenium level. The individual may have a diet high in fat, low in fiber and/or low in fruit, vegetables, poultry and/or fish, may be a current or former tobacco smoker, may have alcohol abuse and/or alcohol dependence problems, be approximately 50 years of age or older, have a lack of physical activity, lead a sedentary lifestyle, or any combination thereof. Individuals with or without a current or prior diagnosis of colorectal cancer may also be interested in other genetic links to phenotypes, and their risk or predisposition to those phenotypes, that are related to colorectal cancer. The individual may have one or more of the following symptoms: blood in stool, blood detected coming from the gastrointestinal tract, weight loss, fatigue, constipation, diarrhea, tenesmus, abdominal pain, hematuria, or pneumaturia. The individual may have a personal medical history of colorectal polyps, precancerous lesion in the colon, colorectal cancer, history of cancer, inflammatory bowel disease, human papilloma virus infection, primary sclerosing cholangitis, exogenous hormone exposure, or any combination thereof. The individual may have a family medical history of colorectal polyps, precancerous lesion in the colon, and/or colorectal cancer.

An individual tested for genetic variants provided in the Prostate Cancer Panel may have their risk or predisposition analyzed for all the phenotypes, or a subset, such as at least 1, 2, 3, 4, 5, 6, or 7 of the following phenotypes: Prostate Cancer; Prognosis with Prostate Cancer (including but not limited to Cancer Aggressiveness, Survival and/or Mortality); Effectiveness and/or Metabolism and/or Choice and/or Dose and/or Adverse Reaction of Medications used to Treat Prostate Cancer; Erectile Dysfunction due to Radiotherapy Treatment for Prostate Cancer; Rectal Bleeding due to Radiotherapy Treatment for Prostate Cancer; Radiosusceptibility and/or Residual DNA Damage Level to Radiation; or Thrombophilia and/or Thromboembolic Disease, determined. A Prostate Cancer Panel can determine the risk or predisposition of a subset of the aforementioned phenotypes, such as at least 1, 2, 3, or 4 of the following phenotypes: Prostate Cancer; Prognosis with Prostate Cancer (including but not limited to Cancer Aggressiveness, Survival and/or Mortality); Effectiveness and/or Metabolism and/or Choice and/or Dose and/or Adverse Reaction of Medications used to Treat Prostate Cancer; or Erectile Dysfunction due to Radiotherapy Treatment for Prostate Cancer. The individual may have abnormal prostate specific antigen (PSA) and/or abnormal early prostate cancer antigen-2 (EPCA-2) levels and/or abnormal sarcosine urine level(s). The individual may be approximately 45 years of age or older, have a diet high in omega-6 fatty acid linoleic acid, have a diet low in vitamin E, omega-3 fatty acids and/or selenium, have low ultraviolet light exposure, have elevated blood levels of testosterone, have ejaculations less than five times per week, or any combination thereof. Individuals with or without a current or prior diagnosis of prostate cancer may also be interested in other genetic links to phenotypes, and their risk or predisposition to those phenotypes, that are related to prostate cancer. The individual may have urinary abnormalities (such as, but not limited to, nocturia, frequency, urgency, hesitancy, intermittency, incomplete voiding, weak urinary stream, or straining), experience weight loss, or any combination thereof. The individual may be overweight or obese and may have a personal or family history of prostate cancer, prostatitis, benign prostatic hyperplasia, or any combination thereof.

An individual tested for genetic variants provided in the Skin Cancer Panel may have their risk or predisposition analyzed for all the phenotypes, or a subset, such as at least 1, 2, 3, 4, 5, or 6 of the following phenotypes: Melanoma; Prognosis with Melanoma (including but not limited to Cancer Aggressiveness, Survival and/or Mortality); Toxicity and/or Effectiveness and/or Dose and/or Choice of Medications used to Melanoma; Wound Dehiscence; Sensitivity to UV Light and/or UV-induced Skin Damage; or Non-melanoma Skin Cancer, determined. A Skin Cancer Panel can determine the risk or predisposition of a subset of the aforementioned phenotypes, such as at least 1, 2, or 3 of the following phenotypes: Melanoma; Prognosis with Melanoma (including but not limited to Cancer Aggressiveness, Survival and/or Mortality); or Toxicity and/or Effectiveness and/or Dose and/or Choice of Medications used to Melanoma. Individuals with or without a current or prior diagnosis of skin cancer, such as melanoma or non-melanoma skin cancer, or precancerous skin lesions, may also be interested in other genetic links to phenotypes, and their risk or predisposition to those phenotypes, that are related to skin cancer, such as melanoma or non-melanoma skin cancer. The individual may have a dermatologic exam that is indicative of precancerous and/or cancerous skin lesion. The individual may have high exposure to ultraviolet light, a lifestyle and/or occupation wherein the individual is often outdoors, or may live in a sunny climate. The individual may have skin lesions (such as, but not limited to, skin irritation or abnormality, such as a wound or abrasion, that does not heal, ulceration, discoloration, and/or changes in existing moles such as change in size, shape, color and/or elevation, or new skin pigmentation), have new moles appearing or have a personal medical history of chronic non-healing wounds. The individual may also have a personal or family history of skin cancer, either melanoma or non-melanoma skin cancer, or both.

An individual tested for genetic variants provided in the Leukemia Panel may have their risk or predisposition analyzed for all the phenotypes, or a subset, such as at least 1, 2, 3, 4, or 5 of the following phenotypes: Leukemia; Prognosis with Leukemia (including but not limited to Survival and/or Mortality); Effectiveness and/or Metabolism and/or Choice and/or Dose and/or Adverse Reaction of Medications used to Treat Leukemia; Prognosis and/or Mortality and/or Graft-versus-Host Disease and/or Bacertemia following Bone Marrow Transplantation and/or Stem Cell Transplantation; or Thrombophilia and/or Thromboembolic Disease, determined. A Leukemia Panel can determine the risk or predisposition of a subset of the aforementioned phenotypes, such as at least 1, 2, 3, or 4 of the following phenotypes: Leukemia; Prognosis with Leukemia (including but not limited to Survival and/or Mortality); Effectiveness and/or Metabolism and/or Choice and/or Dose and/or Adverse Reaction of Medications used to Treat Leukemia; or Prognosis and/or Mortality and/or Graft-versus-Host Disease and/or Bacertemia following Bone Marrow Transplantation and/or Stem Cell Transplantation.

Individuals may select the Lymphoma Panel, which can be used to determine the risk or predisposition of an individual for phenotypes, such as at least 1, 2, 3, 4, or 5 of the following phenotypes: Lymphoma; Prognosis with Lymphoma (including but not limited to Survival and/or Mortality); Effectiveness and/or Metabolism and/or Choice and/or Dose and/or Adverse Reaction of Medications used to Treat Lymphoma; Prognosis and/or Mortality and/or Graft-versus-Host Disease and/or Bacertemia following Bone Marrow Transplantation and/or Stem Cell Transplantation; or Thrombophilia and/or Thromboembolic Disease. A Lymphoma Panel can determine the risk or predisposition of a subset of the aforementioned phenotypes, such as at least 1, 2, 3, or 4 of the following phenotypes: Lymphoma; Prognosis with Lymphoma (including but not limited to Survival and/or Mortality); Effectiveness and/or Metabolism and/or Choice and/or Dose and/or Adverse Reaction of Medications used to Treat Lymphoma; or Prognosis and/or Mortality and/or Graft-versus-Host Disease and/or Bacertemia following Bone Marrow Transplantation and/or Stem Cell Transplantation.

Individuals may be interested in the Leukemia Panel alone, or the Leukemia Panel and Lymphoma Panel. An individual interested in either panel may have an abnormal appearance and/or number of blood cells and/or exposure to radiation and/or carcinogenic substances. The individual may be experiencing one or more of the following symptoms: fatigue, weakness, malaise, petechiae, bruising, bleeding, fever, chills, night sweats, enlargement of one or more lymph nodes, or weight loss. Individuals with or without a current or prior diagnosis of leukemia may also be interested in other genetic links to phenotypes, and their risk or predisposition to those phenotypes, that are related to leukemia. The individual may have a personal medical history of HIV infection and/or Human T-lymphotrophic virus infection, or a personal or family history of leukemia and/or Fanconi anemia.

Individuals can also be interested in the Lymphoma Panel alone. The individual interested in the Lymphoma Panel alone, or the Leukemia & Lymphoma Panel may have an abnormal appearance and/or number of blood cells, splenomegaly, hepatomegaly, anemia, abnormal lymph node biopsy, or any combination thereof. The individual may have had exposure to carcinogenic chemicals (such as, but not limited to, pesticides, solvents, or fertilizers). The individual may also have one or more of the following symptoms: enlargement of one or more lymph nodes, back pain, fever, night sweats, weight loss, pruritus, or fatigue. Individuals with or without a current or prior diagnosis of lymphoma may also be interested in other genetic links to phenotypes, and their risk or predisposition to those phenotypes, that are related to lymphoma. The individual may have a medical history of lymphoma, Epstein-Barr virus infection, Helicobacter bacteria infection, inherited immune deficiency, autoimmune disease, immunosuppressant medication, HIV infection, T-lymphotropic virus type I infection, or any combination thereof. The individual may have a family history of lymphoma, autoimmune disease and/or immune deficiency.

An individual tested for genetic variants provided in the Gastric & Gastrointestinal Cancer Panel can determine the risk or predisposition of an individual for phenotypes, such as at least 1, 2, 3, 4, or 5 of the following phenotypes: Gastric Cancer; Gastric Cancer associated with H. Pylori Infection; Prognosis with Gastric Cancer (including but not limited to Survival and/or Mortality); Toxicity and/or Effectiveness and/or Dose and/or Choice of Chemotherapeutic Medication for Gastrointestinal Cancer; or Thrombophilia and/or Thromboembolic Disease. A Gastric & Gastrointestinal Cancer Panel can determine the risk or predisposition of a subset of the aforementioned phenotypes, such as at least 1, 2, 3, or 4 of the following phenotypes: Gastric Cancer; Gastric Cancer associated with H. Pylori Infection; Prognosis with Gastric Cancer (including but not limited to Survival and/or Mortality); or Toxicity and/or Effectiveness and/or Dose and/or Choice of Chemotherapeutic Medication for Gastrointestinal Cancer. The individual may have an abnormal upper endoscopy suggestive or indicative of precancerous lesion or cancer; a gastric biopsy suggestive or indicative of precancerous and/or cancerous cells, an abnormal upper gastrointestinal radiologic exam, or any combination thereof. The individual may have a diet high in smoked foods, a diet high in salt, a diet low in fruits and vegetables, be a current or former tobacco smoker, have passive exposure to tobacco smoke, or any combination thereof. Individuals with or without a current or prior diagnosis of gastric cancer or gastrointestinal cancer may also be interested in other genetic links to phenotypes, and their risk or predisposition to those phenotypes, that are related to gastric cancer or gastrointestinal cancer. The individual may have one or more of the following symptoms: indigestion, heartburn, abdominal pain, nausea, vomiting, weight loss, weakness, or fatigue. The individual may have a medical history of peptic ulcer disease, helicobacter bacteria infection, gastroesophageal reflux disease, gastritis, or any combination thereof. The individual may have a family history of gastric cancer, peptic ulcer disease, and/or helicobacter bacteria infection.

An individual tested for genetic variants provided in the Head and Neck Cancer Panel can have their risk or predisposition analyzed for all the phenotypes, or a subset, such as at least 1, 2, 3, 4; 5, or 6 of the following phenotypes: Head and Neck Cancer (including Orolaryngeal, Nasopharyngeal, Salivary and/or Esophageal Cancer); Prognosis with Head and Neck Cancer (including but not limited to Survival and/or Mortality); Effectiveness and/or Metabolism and/or Choice and/or Dose and/or Adverse Reactions of Medications used to Treat Head and Neck Cancer; Radiosusceptibility and/or Residual DNA Damage Level to Radiation; Association of Head & Neck Cancer with Alcohol Consumption; or Thrombophilia and/or Thromboembolic Disease, determined. A Head & Neck Cancer Panel can determine the risk or predisposition of a subset of the aforementioned phenotypes, such as at least 1, 2, or 3 of the following phenotypes: Head and Neck Cancer (Including Orolaryngeal, Nasopharyngeal, Salivary and/or Esophageal Cancer); Prognosis with Head and Neck Cancer (including but not limited to Survival and/or Mortality); or Effectiveness and/or Metabolism and/or Choice and/or Dose and/or Adverse Reactions of Medications used to Treat Head and Neck Cancer. The individual may have an abnormal head and/or neck radiologic exam, or a head and/or neck biopsy indicative of precancerous and/or cancerous cells. Individuals with or without a current or prior diagnosis of head and neck cancer may also be interested in other genetic links to phenotypes, and their risk or predisposition of those phenotypes, that are related to head and neck cancer. The individual may be approximately 50 years of age or older, be a current or former tobacco smoker, have passive exposure to tobacco smoke, history of alcohol use, exposure to nickel refining, textiles, woodworking, asbestos, wood dust, paint fumes, and/or petroleum, engage in sexual activity, or any combination thereof. The individual may have one or more of the following symptoms: enlarged lymph node, mass in the neck, sore throat, hoarse sounding voice, weight loss, sinus congestion, numbness of the face, paralysis of the face, or bleeding from the mouth. The individual may have a medical history of head & neck Cancer, Epstein-Barr virus infection, human papilloma virus infection, and/or gastroesophageal reflux disease. The individual may also have a family history of head & neck Cancer, tobacco smoker, and/or exposure to nickel refining, textiles, woodworking, asbestos, wood dust, paint fumes, and/or petroleum.

An individual tested with the Multiple Myeloma Panel can determine the risk or 1, 2, 3, or 4 of the following phenotypes: Multiple Myeloma; Prognosis and/or Mortality and/or Graft-versus-Host Disease and/or Bacertemia following Bone Marrow Transplantation and/or Stem Cell Transplantation; Adverse Reactions and/or Effectiveness and/or Dose and/or Choice of Medication to treat Multiple Myeloma; or Venous Thromboembolism associated with Thalidomide Treatment, determined. The individual may have one or more of the following: an abnormal radiologic exam of a bone, abnormal bone marrow biopsy, monoclonal protein (paraprotein) in their serum and/or urine, abnormal calcium level, abnormal kidney function, renal insufficiency, renal failure, or anemia. Individuals with or without a current or prior diagnosis of multiple myeloma may also be interested in other genetic links to phenotypes, and their risk or predisposition to those phenotypes, that are related to multiple myeloma. The individual may be approximately 50 years of age or older, and had prior or current exposure to toxic chemicals and/or radiation. The individual may be experiencing pain, headache, weakness, and/or fatigue. The individual may have a personal history of one or more of the following infections, or recurrent infections, such as HIV infection, Hepatitis virus infection; or human herpes virus 8 infection. The individual may also have a personal history, or family history of multiple myeloma and/or monoclonal gammopathy of undetermined significance.

Individuals interested in their risk or predisposition to sickle cell, or the risk they have of passing on to their children, or their child's, or other relative's, risk for being a carrier, may be interested in the Sickle Cell Panel. Individuals with or without a current or prior diagnosis of sickle cell trait or sickle cell anemia may also be interested other genetic links to phenotypes, such as conditions, and their risk or predisposition to those phenotypes, that are related to sickle cell. An individual may be tested for their risk or predisposition of an individual for phenotypes, such as at least 1, 2, 3, 4, 5, or 6 of the following phenotypes: Sickle Cell Anemia and/or Sickle Cell Trait; Stroke with Sickle Cell Anemia; Priapism with Sickle Cell Anemia; Modifier of Sickle Cell Anemia Disease and/or Symptomatology and/or Prognosis and/or Hemoglobin F Levels; Analgesic Effectiveness and/or Sensitivity to Pain Medicine and/or Dosage of Pain Medicine Required for Analgesic Effect; or Thrombophilia and/or Thromboembolic Disease. A Sickle Cell Panel can determine the risk or predisposition of a subset of the aforementioned phenotypes, such as at least 1, 2, 3, 4, or 5 of the following phenotypes: Sickle Cell Anemia and/or Sickle Cell Trait; Stroke with Sickle Cell Anemia; Priapism with Sickle Cell Anemia; or Modifier of Sickle Cell Anemia Disease and/or Symptomatology and/or Prognosis and/or Hemoglobin F Levels; or Analgesic Effectiveness and/or Sensitivity to Pain Medicine and/or Dosage of Pain Medicine Required for Analgesic Effect. The individual being tested may have one or more of the following: anemia, abnormal reticulocyte count, target cells, Howell-Jolly bodies, abnormal hemoglobin electrophoresis, or abnormal high performance liquid chromatography (HPLC). The individual may be of African ancestry and may have one or more of the following symptoms: pain; fever, fatigue; or priapism. The individual may have a personal or family history of sickle cell trait or sickle cell anemia.

Individuals interested in the risk or predisposition of passing on or having children with cystic fibrosis, or their child's risk, or other relative's risk, for being a carrier, may be interested in being tested with the Cystic Fibrosis Panel. Individuals with or without a current or prior diagnosis of cystic fibrosis or are a carrier of a cystic fibrosis-related genetic variant, may also be interested in other genetic links to phenotypes, such as conditions, or their risk or predisposition to phenotypes, that are related to cystic fibrosis. An individual may be tested for their risk or predisposition of an individual for phenotypes, such as at least 1, 2, 3, or 4 of the following phenotypes: Cystic Fibrosis; Degree of Pulmonary Disease with Cystic Fibrosis; Susceptibility to Pseudomonas Aeruginosa Infection with Cystic Fibrosis; or Prognosis and/or Severity of Cystic Fibrosis An individual may have an abnormal sweat test, abnormal newborn screening panel, failure to thrive, recurrent lung infections, prenatal diagnosis of cystic fibrosis, or any combination thereof. The individual may be of European ancestry, Jewish heritage, Caucasian ethnicity, or a mix of any combination thereof. An individual may have one or more of the following symptoms: cough, wheezing, hemoptysis, no bowel movement in the first 24-48 hours after birth, diarrhea, stools that are pale or clay colored, delayed growth, failure to thrive, salty-tasting skin, or infertility.

The risk for other rare diseases may be determined by the Rare Disease Screening Panel, which can be used to determine the carrier status, risk or predisposition of an individual for phenotypes, such as at least 1, 2, 3, 4, 5, 6, or 7 of the following phenotypes: Cystic Fibrosis; Glucose-6-phosphate Dehydrogenase Deficiency; Tay-Sachs Disease; Alpha-1 Antitrypsin Deficiency; Retinitis Pigmentosa; Bardet-Biedl Syndrome; or Leber Congenital Amaurosis. A Rare Disease Screening Panel can determine the carrier status, risk or predisposition of a subset of the aforementioned phenotypes, such as at least 1, 2, 3, or 4 of the following phenotypes: Cystic Fibrosis; Glucose-6-phosphate Dehydrogenase Deficiency; Tay-Sachs Disease; or Alpha-1 Antitrypsin Deficiency. This panel, as with all panels listed herein, may be combined with any other panel so that either genetic testing for the genetic variants in this panel or the analysis of the genotypic data pertaining to the phenotypes in this panel, or both, can occur for this panel and any other panels chosen, either separately or together, and the results can be ascertained or conveyed or both, either separately or together.

Panels may also be used for insurance purposes. For example, an individual may be seeking health insurance, life insurance, disability insurance, or other types of insurance, and would like to know their carrier status, risk or predispositions to certain phenotypes, such as conditions. Insurance companies, employers, and others may be interested in knowing an individual's carrier status, risk or predispositions to phenotypes, which may be used to determine factors such as, but not limited to, insurability, health benefits, insurance premiums, insurance plans and various types of coverage. The panels that may be used include the Insurance Panel Alpha, which can be used to determine the risk or predisposition of an individual for phenotypes, such as at least 1, 2, 3, 4, 5, 6, 7, 8, or 9 of the following phenotypes: Longevity and/or Lifespan; Heart Disease (including but not limited to Coronary Artery Disease (CAD) and/or Myocardial Infarction); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer, Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome); Stroke (CVA); Medication Metabolism and/or Adverse Reactions to Medications (Including but not Limited to Pharmacogenomics, Medication Dosing and/or Allergies and/or Choice of Medications and/or Medication Side Effects and/or Adverse Drug Reactions and/or Medication Interactions and/or Malignant Hyperthermia and/or Severe Cutaneous Adverse Reactions and/or Postanesthetic Apnea); Rare Diseases and/or Orphan Diseases and/or Metabolic Diseases and/or Syndromes; or Psychiatric Illness (including but not limited to Depression, Neuroticism, Schizophrenia, Bipolar Disorder, Obsessive-Compulsive Disorder, Panic Disorder, Addictions, Eating Disorders, Suicidality, and/or Personality Disorders). An Insurance Panel Alpha can determine the risk or predisposition of a subset of the aforementioned phenotypes, such as at least 1, 2, 3, 4, or 5 of the following phenotypes: Longevity and/or Lifespan; Heart Disease (including but not limited to Coronary Artery Disease (CAD) and/or Myocardial Infarction); Cancer (including but not limited to Lung Cancer, Colorectal Cancer, Breast Cancer, Ovarian Cancer, Cervical Cancer, Prostate Cancer, Gastric Cancer, Skin Cancer, Head and Neck Cancer, Bone Cancer, Muscle Cancer, Pancreatic Cancer, Liver Cancer; Thyroid Cancer, Parathyroid Cancer, Adrenal Cancer, Kidney Cancer, Bladder Cancer, Uterine Cancer, Endometrial Cancer, Retinoblastoma, Germ Cell Tumors, Testicular Cancer, Brain Cancer, Gastroenteropancreatic Neuroendocrine Tumors, Leukemia, Lymphoma, Multiple Myeloma, and/or Cancer Syndromes) and/or Precancerous Lesions; Chronic and/or Degenerative and/or Fatal Neurologic Disease (Including but not Limited to Alzheimer's Disease, Parkinson Disease, Huntington's Disease, Amyotrophic Lateral Sclerosis, Transmissible Spongiform Encephalopathies, Creutzfeldt-Jakob Disease, variant Creutzfeldt-Jakob Disease, Gerstmann-Sträussler-Scheinker Syndrome, Fatal Familial Insomnia, and/or Kuru); or Cardiac Arrhythmia and/or Cardiac Conduction Abnormality (including but not limited to Atrial Fibrillation, Ventricular Fibrillation, Re-entry Arrhythmias, Arrhythmogenic Right Ventricular Dysplasia, Hypertrophic Cardiomyopathy, Wolff-Parkinson-White Syndrome, Brugada Syndrome, Tachycardias, Heart Blocks, Long QT Syndrome, Short QT Syndrome, Sick Sinus Syndrome, Sudden Unexplained Nocturnal Death Syndrome and/or Sudden Infant Death Syndrome).

In some embodiments, the Insurance Panel Alpha may be used along with the Insurance Panel Beta, or the Beta panel may be used alone, which can be used to determine the risk or predisposition of an individual for phenotypes, such as at least 1, 2, 3, 4, 5, 6, 7, 8, or 9 of the following phenotypes: Longevity and/or Lifespan; Myocardial Infarction; Lung Cancer; Diabetes Mellitus, Type II and/or Insulin Resistance s; Multiple Sclerosis; Crohn Disease; Fibromyalgia; Stroke (CVA); or Alzheimer's Disease. The Insurance Panel Beta can determine the risk or predisposition of a subset of the aforementioned phenotypes, such as at least 1, 2, 3, 4, 5, or 6 of the following phenotypes: Longevity and/or Lifespan; Myocardial Infarction; Lung Cancer; Diabetes Mellitus, Type II and/or Insulin Resistance s; Multiple Sclerosis; or Crohn Disease.

The number of panels, such as those described above, or number of phenotypes, such as described above, or number of genetic variants, or number of genes or loci, or various combinations thereof, may be used to determine the level of service or price for determining an individual's risk or predisposition to or carrier status of various genetic variants or phenotypes or both. If a single panel is chosen, the sample for an individual that supplies the genetic material (such as buccal tissue, epithelial tissue, saliva, blood, hair, hair follicle, skin, etc.) may be used to test only that panel, or it may be used on a number of panels, but the results from only that panel is reported to the individual. If a subset of a panel is used, the individual's sample may be tested for only the phenotypes, such as conditions, chosen by the individual, or their sample may be used for the entire panel or a number of panels, but only the results from the phenotypes, such as conditions, chosen by the individual are reported. Results from genetic variants, phenotypes, such as conditions, or panels not chosen by the individual initially may be released to the individual if the individual chooses so at a later date. The individual may have to pay an additional cost. The individual may or may not submit another biologic sample. The conditions selected can be selected not only by the individual who's risk or predisposition or carrier status is being tested for, but can also be selected by another party, such as a parent, guardian, relative, health care manager, medical professional, or third party with the authority to, or may be selected by the individual with consultation of the aforementioned parties, or selected by the aforementioned parties with the consultation of the individual being tested.

Different levels of service with varying costs can also be provided, for example, an initial analysis by a general practitioner or managing doctor and a GC or a higher level of service where the initial analysis is followed with a consultation with a medical specialist, such as an alternative medicine specialist, anesthesiologist, cardiologist, complementary medicine specialist, dental or oral specialist, dermatologist, endocrinologist, gastroenterologist, hepatologist, hematologist, infectious disease specialist, immunologist, fertility specialist, medical geneticist, men's health specialist, nutrition and obesity specialist, neurologist, nurse practitioner, psychologist, obstetrician, gynecologist, oncologist, ophthalmologist, pain medicine specialist, pediatrician, pharmacologist, physical therapist, psychiatrist or addiction specialist, physician assistant, pulmonologist, rheumatologist, surgeon, urologist, and women's health specialist. An individual can choose a lower level of testing, for example, a single phenotype, such as a disease or condition, or a single genetic variant or single gene, and afterwards, decide to obtain a more comprehensive genetic profile or a full genetic profile. An individual may choose to have an initial consultation with a managing doctor, and decide to have a consultation with a specialist referred to by the managing doctor or genetic counselor or physician assistant or nurse practitioner or other healthcare professional. In some cases, non-medical specialists may be consulted as well, either in conjunction with a medical specialist or independently. For example, the individual may consult with one or more of the following: an acupuncturist, a chiropractor, a herbologist, a masseuse, a weight loss clinic or spa employee or healthcare provider, health or medical spa employee, hotel or resort or casino employee or healthcare provider, cruise-ship employee or healthcare provider, space flight or space travel specialist or representative, dating specialist, a professional match-maker (including a dating web-site), a relationship specialist, a therapist, a therapist, a personal trainer, a fitness/exercise professional such as a fitness trainer, an athletic coach, a dance coach, an addiction counselor or sponsor, a teacher, a learning specialist, a life coach, a spiritual advisor, an advisor, a religious or spiritual cleric, and a professional organizer.

After an individual selects a genetic profile or genetic testing level, such as number of panels, choice of panel(s), number of genetic variants, number of genes or loci, number of phenotypes, such as conditions, and/or reflex testing, and/or the degree or depth or level of reflex testing, and/or OP-CADI, the individual may sign a waiver or other release or disclaimer and a biological sample is obtained from the individual for genetic testing. The sample may be linked to a number, such as a “Confidential Client Number” or CCN, which is given to the individual or the individual's healthcare provider or the person who ordered the test or other third party with authority to order the test or have access to the CCN. The individual's name and CCN can be encrypted and a single or multiple physical (non-electronic) copy or copies or electronic copy or copies of the information linking the individual's name to the CCN can be kept to maintain confidentiality.

Genetic samples can be obtained from kits provided to individuals with sample collection containers for the individual's biological sample. The kit may also provide instructions for an individual to directly collect their own sample, such as how much hair, urine, sweat, buccal tissue, tongue cells, or saliva to provide. The kit may also contain instructions for an individual to request tissue samples to be taken by a health care specialist, or instructions for how they can obtain their own biologic sample (as discussed above), to be stored indefinitely for the purpose of DNA banking (long-term storage of DNA for future use, such as analysis, or to pass on to future generations). The kit may also provide return packaging for the sample to be sent to a sample processing facility, where the individual's genetic material is then isolated from the biological sample for either storage or for genetic testing or both.

A genetic sample of DNA or RNA may be isolated from a biological sample according to any of several well-known biochemical and molecular biological methods, see, e.g., Sambrook, et al., Molecular Cloning: A Laboratory Manual (Cold Spring Harbor Laboratory, New York) (1989). There are also several commercially available kits and reagents for isolating DNA or RNA from biological samples, such as those available from DNA Genotek, Gentra Systems, Qiagen, Ambion, and other suppliers. Buccal sample kits are readily available commercially, such as the MasterAmp™ Buccal Swab DNA extraction kit from Epicentre Biotechnologies, as are kits for DNA extraction from blood samples such as Extract-N-Amp™ from Sigma Aldrich. DNA from other tissues may be obtained by digesting the tissue with proteases and heat, centrifuging the sample, and using phenol-chloroform to extract the unwanted materials, leaving the DNA in the aqueous phase. The DNA may then be further isolated by ethanol precipitation.

DNA may be collected using DNA self collection kit technology available, such as from DNA Genotek, an individual collects a specimen of saliva for clinical processing. The sample conveniently may be stored and shipped at room temperature. After delivery of the sample to an appropriate laboratory for processing, DNA is isolated by heat denaturing and protease digesting the sample, typically using reagents supplied by the collection kit supplier at 50° C. for at least one hour. The sample is next centrifuged, and the supernatant is ethanol precipitated. The DNA pellet is suspended in a buffer appropriate for subsequent analysis. In some embodiments, the sample is obtained from a cheek swab.

A DNA sample may also be provided to the individual in a form visible to the human eye. This visible DNA can be placed in a sealed container, tube, vial, locket, charm, watch, necklace, mantle-piece, show-piece or other display casing that may or may not be part of the genetic report or given to the individual at the same time as the genetic report or both. The visible DNA may also be combined with a coloring or fluorescent agent in order to make it more visible against its background. This service may be at an additional cost. The DNA may be made visible either using a laboratory kit, such as EPICENTRE® Biotechnologies' MasterPure™ Complete DNA and RNA Purification Kit, Axygen Biosciences' AxyPrep™ Multisource Genomic DNA Miniprep Kit, Whatman's GenSpin™ Genomic DNA Purification Kit, or any other commercially available kits, or by using methods known in the arts, such as by the following methodology: placing saliva or buccal tissue into 3-5 milliliters of water in a container (saliva can be obtained by swishing the 3-5 ml of water around in the mouth and then spitting into a container), add approximately 1-2 teaspoons of salt, then add in 1-2 milliliters of household dish soap, sir for approximately five minutes, then add 4-5 milliliters of denatured alcohol and wait 10-20 minutes. An individual may be interested in displaying their DNA. This visible DNA can be shipped back to the individual directly or handed to them in person, either by their physician, healthcare provider, genetic counselor, nurse practitioner, physician assistant, or other person who ordered the test, either along with their genetic report or at a separate time. This visible DNA may be ordered along with genetic analysis or on its own. This service may incur an additional fee.

DNA art may also be provided to the individual in the form of pictures or drawings that show either part of their genetic code or their entire genetic code. The picture may be in the form of a black and white or color picture, photograph, image, or print out (such as of the data) of the array, microarray, massarray, beadarray, genechip, or sequencing (such as, for example, by utilizing shotgun sequencing, double-barrel shotgun sequencing, pyrosequencing, nanopores, fluorophores, or nanoballs) results from genetic testing that was conducted for that individual or for the individual's family, such as based on the panel they ordered, or may be a depiction or representation of part or the whole genome, such as their entire genetic code or a part of their genetic code, such as if sequencing or full genome sequencing is utilized. The pictures, photographs, video images, computer images, drawings, sketches, or any other images depicting an individual's or a family's genetic code, such as for a single genetic variant, multiple genetic variants, single gene, multiple genes, single locus, multiple loci, single chromosome, multiple chromosomes, partial genome, or full genome (such as sequence data, restriction fragment length polymorphisms, gel electrophoresis products, etc.) may be digital, printed, screened, via decalcomania, via holography, drawn, painted, woven, such as into rugs, carpets or tapestries, and blown, such as with glassblowing, and may range in size from very small, such as wallet-size, such as one inch by one inch, to extremely large, such as billboard size or building wall size, such as 100 feet by 100 feet. DNA may be produced by the company that conducts the genetic testing or the genetic analysis, the laboratory (158) that processes the genetic sample and the genetic testing, or by another company, such as DNA 11 Inc. (Ottawa, Ontario, Canada). The DNA art may be produced from the same genetic sample that is used for genetic testing and/or analysis or it may be from a different genetic sample. The DNA art may appear within the genetic report, such as on the cover of the genetic report or within the genetic report, or be delivered with or around the same time as the genetic report, or at an earlier or later date, and may be ordered at the same time that the genetic testing, genetic analysis or genetic report is ordered or it may be ordered at a later time (utilizing either the same genetic material or new genetic material) or it may be ordered separately, on its own. This service may incur an additional fee.

RNA may also be used as the genetic sample. In particular, genetic variations that are expressed can be identified from mRNA. The term “messenger RNA” or “mRNA” includes, but is not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a DNA amplified from the cDNA, an RNA transcribed from the amplified DNA, etc., are all derived from the mRNA transcript. RNA may be isolated from any of several bodily tissues using methods known in the art, such as isolation of RNA from unfractionated whole blood using the PAXgene™ Blood RNA System available from PreAnalytiX. Typically, mRNA may be used to reverse transcribe cDNA, which may then be used or amplified for gene variation analysis.

The methods described herein can be applied in the context of any platform capable of genotyping a sample, e.g., arrays, microarrays, massarrays, beadarrays, genechips, PCR-based techniques, exome sequencing, full (such as whole) exome sequencing, partial genome sequencing or full (such as whole) genome sequencing, such as with shotgun sequencing, double-barrel shotgun sequencing, pyrosequencing, nanopore sequencing (nanopores), fluorophore sequencing (fluorophores), DNA nanoball sequencing (nanoballs), or any other partial or full (such as whole) genome sequencing technologies: The terms “sequencing apparatus” and “sequencing platform” include but are not limited to arrays, nanopores, nanoballs, pyrosequencing, shotgun sequencing, double-barrel shotgun sequencing, SMRT™ Sequencing Technology or any other method of genetic sequencing that identifies the allele or genotype of one or more genetic variants in a genome. Often, results or data from a sequencing apparatus or a sequencing platform are provided as part of sequencing services. Prior to identifying a genetic variant, such as a polymorphism, through testing or analysis or both, a genetic sample is typically amplified, either from DNA or cDNA reverse transcribed from RNA, although other genetic testing methodologies may exist that may not require DNA amplification. DNA may be amplified by a number of methods, many of which employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al, Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86:1173-1177 (1989) and WO88/10315), self-sustained sequence replication (Guatelli et al., Proc. Nat. Acad. Sci. USA, 87:1874-1878 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) nucleic acid based sequence amplification (NABSA), rolling circle amplification (RCA), multiple displacement amplification (MDA) (U.S. Pat. Nos. 6,124,120 and 6,323,009) and circle-to-circle amplification (C2CA) (Dahl et al. Proc. Natl. Acad. Sci 101:4548-4553 (2004)). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 5,409,818, 4,988,617, 6,063,603 and 5,554,517 and in U.S. Ser. No. 09/854,317, each of which is incorporated herein by reference.

Several methods are known in the art to identify genetic variations and include, but are not limited to, DNA genotyping or sequencing or both by any of several methodologies, such as arrays, microarrays, genechips, bead arrays, massarrays, PCR based methods, nanopores, nanoballs, fluorophores, pyrosequencing, shotgun sequencing, double-barrel shotgun sequencing, sequencing by ligation, sequencing by synthesis, fragment length polymorphism assays (restriction fragment length polymorphism (RFLP), cleavage fragment length polymorphism (CFLP), cleaved amplified polymorphism (CAP)), hybridization methods using an allele-specific oligonucleotide as a template (e.g., TaqMan PCR method, the invader method, the DNA chip method), methods using a primer extension reaction, mass spectrometry (MALDI-TOF/MS method), fluorescence in situ hybridization, karyotyping, and the like.

A low density or mid density or high density DNA array, such as a microarray or massarray or bead array or genechip, can be used for genetic variant, such as SNP, identification, to generate a genotype(s) for one or more genetic variants (genetic testing that leads to a raw genotype profile for an individual) and profile generation. Such arrays or microarrays or bead arrays are commercially available, for example, from Affymetrix and Illumina (see Affymetrix GeneChip® 500K Assay Manual, Affymetrix, Santa Clara, Calif. (incorporated by reference); Sentrix® humanHap650Y genotyping beadchip, Illumina, San Diego, Calif.). In these assays, a subset of the human genome can be amplified through a single primer amplification reaction using restriction enzyme digested, adaptor-ligated human genomic DNA. The sample is denatured, labeled, and then hybridized to a microarray with small DNA probes at specific locations on a coated quartz surface. The amount of label that hybridizes to each probe as a function of the amplified DNA sequence is monitored, thereby yielding sequence information and resultant SNP identification. For example, use of the Affymetrix GeneChip 500K Assay is carried out according to the manufacturer's directions. Briefly, isolated genomic DNA is first digested with either a NspI or StyI restriction endonuclease. The digested DNA is then ligated with a NspI or StyI adaptor oligonucleotide that respectively anneals to either the NspI or StyI restricted DNA. The adaptor-containing DNA following ligation is then amplified by PCR to yield amplified DNA fragments between about 200 and 1100 base pairs, as confirmed by gel electrophoresis. PCR products that meet the amplification standard are purified and quantified for fragmentation. The PCR products are fragmented with DNase I for optimal DNA chip hybridization. Following fragmentation, DNA fragments should be less than 250 base pairs, and on average, about 180 base pairs, as confirmed by gel electrophoresis. Samples that meet the fragmentation standard are then labeled with a biotin compound using terminal deoxynucleotidyl transferase. The labeled fragments are next denatured and then hybridized into a GeneChip 250K array. Following hybridization, the array is stained prior to scanning in a three step process consisting of a streptavidin phycoerythrin (SAPE) stain, followed by an antibody amplification step with a biotinylated, anti-streptavidin antibody (goat), and final stain with streptavidin phycoerythrin (SAPE). After labeling, the array is covered with an array holding buffer and then scanned with a scanner such as the Affymetrix GeneChip Scanner 3000. Analysis of data following scanning of an Affymetrix GeneChip Human Mapping 500K Array Set is performed according to the manufacturer's guidelines.

As an alternative to, or in addition to, DNA array, microarray, massarray, or bead array analysis, genetic variations such as SNPs, DIPs and mutations can be detected by DNA sequencing. DNA sequencing may also be used to sequence a small portion (such as one full gene or a portion of one gene), a substantial portion (such as multiple genes or multiple chromosomes), or the entire genomic sequence of an individual. Traditionally, common DNA sequencing has been based the technique known as Sanger sequencing which uses polyacrylamide gel fractionation to resolve a population of chain-terminated fragments (Sanger et al., Proc. Natl. Acad. Sci. USA 74:5463-5467 (1977)). Alternative methods have been and continue to be developed to increase the speed and ease of DNA sequencing. For example, high throughput and single molecule sequencing platforms are commercially available or under development from 454 Life Sciences (Branford, Conn.) (Margulies et al., Nature (2005) 437:376-380 (2005)); Solexa (Hayward, Calif.), acquired by Illumina, Inc. (San Diego, Calif.); Helicos BioSciences Corporation (Cambridge, Mass.) (U.S. application Ser. No. 11/167,046, filed Jun. 23, 2005), and Li-Cor Biosciences (Lincoln, Nebr.) (U.S. application Ser. No. 11/118,031, filed Apr. 29, 2005). Shotgun sequencing and double-barrel shotgun sequencing are also sequencing methods. Nanopore sequencing (nanopores) is one such method that may allow for high throughput DNA sequencing (Vercoutere, W. et al. Nature Biotechnology 19, 248-252 (2001), Sauer-Budge, A. F. et al. Phys. Rev. Lett. 90, 238101-238101-238101-238104 (2003), Howorka, S. Nat. Biotechnol. 2001 July; 19(7):636-9). Nanopores may be used to sequence a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif.), Oxford Nanopore Technologies LTD (Kidlington, United Kingdom), and Agilent Laboratories (Santa Clara, Calif.). Nanopore sequencing methods and apparatus have been described in the art and for example are provided in U.S. Pat. No. 5,795,782, herein incorporated by reference in its entirety. Other sequencing technologies include nanoballs, fluorophores and Single Molecule Real Time DNA sequencing technology (SMRT™) technology, and pyrosequencing, as described in U.S. Pat. Nos. 7,371,851; 7,405,281; 7,170,050; 7,244,567; 7,244,559; 7,264,929; 7,323,305; 7,211,390; and 7,335,762; and in US Patent Application Publication Nos. US2009/0053724; US2007/0231804; US2009/0024331; US2008/0206764; US2009/0011943; US2009/0005252; and US2008/0171331; US2008/0213771 herein incorporated by reference in their entirety.

For example, a low-, medium- or high-density array, such as the commercially available platforms from Sequenom or Affymetrix or Illumina, is used for genetic variant identification and profile generation. As technology evolves, there may be other technology vendors who can generate low-, medium- or high-density genotype (such as genetic variant or polymorphism or mutation or copy number variation) profiles. The massarray or microarray or beadarray can have at least 1000, 5000, 6,000, 6,500, 7,000, 8,000; 10,000, 15,000, 20,000, 25,000, 30,000, 45,000, or 50,000 unique oligonucleotide sequences. Each oligonucleotide sequence may exist one or more times on the array, such as for redundancy or to increase accuracy, as the same (non-unique) oligonucleotide sequence may test for the same genetic variant and therefore these oligonucleotide sequences (which test for the same exact genetic variant) are not unique; only oligonucleotide sequences that test for or detect different genetic variants are considered herein unique oligonucleotide sequences. A genetic variant may be unique if it exists in a different location within the genome, even if it is just one basepair away from another genetic variant, or it may also be unique if it occurs at the same exact location within the genome but encompasses a different type of variation, such as a different nucleotide change. For example, if two genetic variants occur at the same exact location but one is the change from an Adenine (A) to a Guanine (G) and the other is a change from an Adenine (A) to a Thymine (T), then this constitutes a unique genetic variant and two, unique probes, such as oligonucleotide sequences, may be needed to detect these changes (one unique oligonucleotide sequence to test for or detect the A to G change and the other unique oligonucleotide sequence to test for or detect the A to T change). Each of the unique oligonucleotide sequences corresponds to, or is associated with, a genetic variant; such as a genetic polymorphism, such as a SNP. For example, each sequence may be associated with a phenotype that is medically relevant or linked to at least one phenotype as reported in published literature. For example, an oligonucleotide sequence can comprise a genetic variant, be a sequence in linkage disequilibrium with a genetic variant, such as a SNP, or contain genetic sequence immediately flanking the genetic variant of about 5, 10, 15, 20, 25, 30, 35, 40, 45, 50, 60, 75, 100, or more bps upstream or downstream of a genetic variant. The genetic variant may be medically related or non-medically related. The genetic variant may be trait related or non-trait related. In other embodiments, each of the unique oligonucleotide sequences on an array is associated with a genetic variant, such as a polymorphism or mutation that is medically relevant or provides information about a trait, for example, each sequence on the array is associated with a SNP that is correlated with a disease or condition or trait. For example, the sequences on the array may be used to detect the genetic variants in the non-limiting examples of representative genes listed in Table 4. Other non-limiting examples of representative genes may include those listed in FIG. 15-18. The array may comprise sequences that detect a genetic variation, such as a SNP, in each of the non-limiting examples of representative genes listed in Table 4, or those listed in FIG. 15-18, that is associated with a genetic condition or phenotype. Some arrays may have oligonucleotide sequences, wherein at least 50, 70, 75, 80, 85, 90, or 95% of the sequences are associated with a genetic variant that is medically relevant. In some embodiments, at least 5, 10, 15, 20, 25, 30, 50, 75, 100, 125, 150, 175, 200, 250, 300, 400, 500, 1000 or more unique phenotypes are associated with the genetic variants on the array. In some embodiments, each of the oligonucleotides on the array is associated with a different genetic variant or a different phenotype, such as a disease or condition. In some arrays, different oligonucleotides may be associated with the same phenotype, such as a disease or condition.

Arrays can also have oligonucleotide sequences wherein at least 5, 10, 25, 50, 65, 70, or 75% of the sequences corresponding to a genetic variant, such as SNP, are unique sequences or sequences not listed in a public database, for example sequences immediately flanking the genetic variant that are about 5, 10, 20, 25, 30, 35, 40, 45, 50, 60, 75, 100, 200, or more bps upstream or downstream of a genetic variant. The oligonucleotides on an array may detect at least about 100, 1000, 5000, 6,000, 6,500, 7,000, 8,000, 10,000, 15,000, 20,000, 25,000, 30,000, 45,000, 50,000, 100,000, 150,000, 200,000, 250,000, 300,000, 350,000, 400,000, 450,000, 500,000, 750,000, 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000 or more genetic variants, such as SNPs. The number of genetic variants may be present in at least approximately 100, 250, 500, 750, 1000, 1250, 1500, 2000, 3000, 3500, 4000, 4500, 5000, 5500, 6000, 6500, 7000, 7500, 8000, 8500, 9000, 9500, 10,000, 10,500, 11000, 11500, 12000, 12500, 13,000, 13,500, 14,000, 14,500, 15,000, 15,500, 16,000, 16,500, 17,000, 17,500, 18,000, 18500, 19000, 19500, or 20,000 genes. In some embodiments, each sequence on an array is used to determine or calculate an organ system score. In other embodiments, each of the sequences is used to determine or calculate at least 2 or more organ system scores. Some arrays contain sequences wherein each of the sequences is used to determine or calculate a score for a medical specialty. In yet other embodiments, each of the sequences are used to calculate or obtain an overall genetic health score. In some embodiments, the array comprises unique oligonucleotide sequences that detect at least 5000 medically-relevant genetic variants or SNPs, at least 6000 medically-relevant genetic variants or SNPs, or at least 6500 medically-relevant genetic variants or SNPs. Medically-relevant genetic variants or SNPs refer to a genetic variant that has been associated or linked in published literature, such as a published journal article, with either an increased or decreased risk or predisposition to disease or medical condition or associated with being a carrier or affected or likely-affected by a disease or medical condition. The number of unique medically relevant phenotypes associated with genetic variants that the unique oligonucleotides may be able to test for or detect may include at least 75, 100, 150, 200, 250, 300, 350, 400, 450, 500, 550, 600 or more phenotypes.

The genetic variants, such as SNPs, detected by the array may be present in non-coding regions. The genetic variants, such as SNPs, may be medically related or non-medically related. The genetic variants, such as SNPs, may include only clinically relevant genetic variants, or genetic variants in genes or in linkage disequilibrium of other genetic variants, correlated with clinical phenotypes, such as diseases or medical conditions. The SNPs, or other genetic variants, may be organized by medical specialty, gene, location on a chromosome, phenotype or disease. The SNPs, or other genetic variants, can be organized by clinical severity or by how well that genetic variant is thought to correlate with a specific phenotype, such as a disease or condition. The private database can also have precise information for each genetic variant, such as a SNP. For example, information such as odds ratio or other risk value, applicable ethnicities or populations, inheritance patterns, journal references, journal links, brief SNP (or other genetic variant) synopsis, phenotype-associated allele or genotype, p-value of the association, confidence interval of the risk value, incidence or prevalence of the phenotype, frequencies of the alleles or genotypes, or both, of the genetic variant, different scoring systems for the genetic variant-phenotype association (as previously discussed), such as the GVP score, and recommendations.

In some embodiments, each of the genetic variants detected by the array is medically relevant. In some embodiments, each of the sequences on the array is linked to a journal reference or a recommendation, directly or indirectly (for example, if the sequence is linked to a condition, wherein the condition is linked to a journal reference or recommendation or both). In other embodiments, each of the sequences on the array is for a specific phenotype, such as a disease, or for a specific genetic testing, such as for children, for carrier information, or for cancer patients. The array can have sequences wherein each sequence, or a subset of the sequences, is used to determine the pharmacogenomic profile of an individual.

For example, each sequence on an array, or a subset of sequences on the array may be used to determine the risk of phenotypes, such as conditions, or carrier status or both in the panels listed in FIG. 15-18, or of a Custom Panel (FIG. 19). A single array may represent multiple panels (2, 3, 4, 5, 6, 7, 8, 9, 10, 11, 12, 13, 14, 15 or more) or may represent a single panel, such as, but not limited to, a Full Genome Panel Alpha (FIG. 15), Full Genome Panel Beta, Pediatric Panel Alpha, Pediatric Panel Beta, Women's Health Panel Alpha, Women's Health Panel Beta, Men's Health Panel Alpha, Men's Health Panel Beta, Executive Panel Alpha, Executive Panel Beta, Golden Panel Alpha [Geriatric and Aging Panel Alpha], Golden Panel Beta [Geriatric and Aging Panel Beta], Carrier Screening Panel, Embryo and Fetus Panel Alpha, Embryo and Fetus Panel Beta, Female Fertility Panel, Male Fertility & Erectile Function Panel, Pregnancy Panel, Assisted Reproductive Technology Panel, Reproduction, Egg & Sperm Donor Screening Panel Alpha, Reproduction, Egg & Sperm Donor Screening Panel Beta, Sexuality, Mate Selection, Relationships and Marriage/Divorce Panel, Exercise, Fitness and Athletic Training Panel, Dietary, Nutrition & Weight Management Panel Alpha, Dietary, Nutrition & Weight Management Panel Beta, Longevity Panel Alpha, Longevity Panel Beta, Illness of Unknown Etiology Panel, Military and Armed Forces Panel Alpha (FIG. 16), Military and Armed Forces Panel Beta (FIG. 17), Law Enforcement/Forensic/Investigative Panel (FIG. 18), Emergency Panel, Cardiovascular Panel Alpha, Cardiovascular Panel Beta, Dermatology Panel, Gastroenterology Panel, Neurology Panel, Neurologic Disease of Unknown Etiology Panel, Mouth & Dental Panel, Surgery & Anesthesiology Panel, Transplant Panel, Gynecology Panel, Auditory Panel, Endocrinology Panel, Rheumatology Panel Alpha, Rheumatology Panel Beta, Urology & Nephrology Panel, Opthalmology Panel, Oncology Panel, Adult Psychiatry Panel, Pediatric Psychiatry Panel, Addiction Panel, Infectious Disease Panel, World Infectious Disease Panel, Pulmonology Panel, Sleep Medicine Panel, Palliative Care Panel, Insurance Panel Alpha, Insurance Panel Beta, HIV Panel, Autism Panel, Learning & Education Panel, Heart Failure Panel, Preterm Infant Panel, Newborn Panel Alpha, Newborn Panel Beta, Multiple Sclerosis Panel, Depression Panel, Schizophrenia Panel, Bipolar Panel, Eating Disorder Panel, Smoker's Panel, Drinker's Panel, Allergy and Atopy Panel, Pharmacology & Alternative Medication Panel, Miscarriage, Spontaneous Abortion, or Difficulty Conceiving Panel, Pain Panel, Breast Cancer Panel, Ovarian Cancer Panel, Lung Cancer Panel, Colorectal Cancer Panel, Prostate Cancer Panel, Skin Cancer Panel, Leukemia Panel, Lymphoma Panel, Gastric & Gastrointestinal Cancer Panel, Head & Neck Cancer Panel, Multiple Myeloma Panel, Sickle Cell Panel, Cystic Fibrosis Panel, Coronary Artery Disease Panel, Myocardial Infarction Panel, Lipid Level Panel, Blood Pressure Panel, Obesity Panel, Diabetes Mellitus (Type II) Panel, Diabetes Mellitus (Type I) Panel, Inflammatory Bowel Disease Panel, Gastrointestinal Disease of Unknown Etiology Panel, Viral Hepatitis Panel, Alzheimer's Disease Panel, Parkinson Disease Panel, Seizure & Epilepsy Panel, Thyroid Panel, Osteoarthritis Panel, Rheumatoid Arthritis Panel, Systemic Lupus Erythematosus Panel, Gout Panel, Malaria Panel, Asthma Panel, Chronic Obstructive Pulmonary Disease Panel, Pulmonary Hypertension Panel, Polycystic Ovary Syndrome Panel, Stroke Panel, Autoimmune Panel, Behavior & Aptitude Assessment Panel, Kidney Transplant Panel, Liver Transplant Panel, Lung Transplant Panel, Stem Cell Transplant Panel, Infection Panel, Blood Flow, Thrombosis and Thromboembolism Panel, Sports Panel, Pathology & Tissue Repository Panel, Incarceration Panel Research & Clinical Trial Panel, Close Living Quarters Panel, Rare Disease Screening Panel, Medical Procedure & Interventional Radiology Panel, Fibromyalgia Panel, Heartbeat/Arrhythmia Panel, Blood Panel, Dyslipidemia Panel, Death/Autopsy Panel. There are also Custom Panels, where an individual can choose any disease or trait from any of the panels described herein (such as FIGS. 15-18). An individual can choose different denominations, such as a Custom 10 Panel, which tests for 10 phenotypes or a Custom 20 Panel, which tests for 20 phenotypes. Custom panels can range from one phenotype to over 1,000 phenotypes. The Custom Panel may have approximately, 5, 10, 15, 20, 25, 30, 40, 50, 60, 75, 100, 150, 200, 250, 300, 400, 500, 1000 or more phenotypes, such as diseases or traits.

A single array (meaning any type of genetic testing array, such as microarray, massarray, genechip or beadarray) may comprise sequences used to determine the degree of risk of phenotypes, such as diseases or traits, or carrier status, or both, on a number of panels, or all of the panels. Alternatively, the degree of risk or predisposition to the phenotypes, such as diseases or traits, or carrier status, or both on one or more panels can be determined using any platform capable of genotyping a sample, e.g., microarrays, massarrays, bead arrays, PCR-based techniques, exome sequencing, or genome sequencing including partial or full genome sequencing, such as nanopore sequencing (herein referred to as nanopores).

Each panel can be used to detect all the phenotypes, such as diseases or traits, listed for each panel, as shown in FIG. 15-18. Panels may also comprise a subset of phenotypes, such as diseases or traits, as listed.

As illustrated in FIG. 1, the results (120), obtained from the genetic sample, for example, obtained by microarray analysis or sequencing analysis, can be sent to the central location (104), and analysis of the isolated genetic sample and generation of a raw (unanalyzed in terms of associations with phenotypes) genetic genotype profile is performed. The results and statistical analysis (such as p-values, accuracy, reproducibility, reliability or any other relevant values for each of the genetic variants detected) can be transmitted at step (122), (124), or (126) to a location, for example, where an individual submitted their sample. The raw genotype results and statistical analysis may then be stored, partially analyzed or fully analyzed in order to generate a genetic report (report) to present to the individual (as described further below). The report, results and analysis can be transmitted securely over a network. Alternatively, the report may not be transmitted at all over a network, but is printed in hard copy and stored in hard copy only such that no electronic version is stored. Alternatively, an electronic version is stored, but only with an identification number (such as the CCN). Three different types of reports may be generated, one for a GC, one for the physician or the person or entity that ordered the genetic profile, and one for the individual, wherein each report is tailored to each person reading the report (who that specific report was sent or given to), respectively.

FIG. 11A is a block diagram showing a representative example logic device through which results can be received and analyzed to generate a report. FIG. 11A shows a computer system (or digital device) 800 to receive and store results, analyze the results, and produce a report of the results and analysis. The computer system 800 may be understood as a logical apparatus that can read instructions from media 811 and/or network port 805, which can optionally be connected to server 809 having fixed media 812. The system shown in FIG. 11A includes CPU 801, disk drives 803, optional input devices such as keyboard 815 and/or mouse 816 and optional monitor 807. (Parts 800, 801, 803, 805, 807, 811, 815 and 816 are also depicted in FIG. 11B). Data communication can be achieved through the indicated communication medium to a server 809 at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or for review by a party 822. The receiving party 822 can be but is not limited to an individual, a health care provider or a health care manager. In one embodiment, a computer-readable medium includes a medium suitable for transmission of a result of an analysis of a biological sample. The medium can include a result regarding analysis of an individual's genetic profile, wherein such a result is derived using the methods described herein.

FIG. 11B is a schematic of a non-limiting example of the general steps for obtaining a genetic analysis of a sample obtained from an individual; the example includes a computer system that can be used for receiving, storing, and analyzing data from genotyping, genetic testing and/or genetic analysis.

In step 902 of FIG. 11B, a sample of genetic material 904 of an individual is obtained or isolated from a biological sample of an individual (e.g. blood, hair, skin, saliva, semen, buccal cells, epithelial tissue, and various bodily tissues). The genetic sample 904 may be obtained by a variety of methods known in the art. In step 906 raw genetic data (e.g. genomic sequence, SNP profiles, etc.) are stored on the computer 800 (also described in FIG. 11A). All or a portion of the data may be input by a user interface such as a mouse 816 (also described in FIG. 11A) or a keyboard 815 (also described in FIG. 11A). Alternatively, the computer may be connected to the genotyping or genome sequencing apparatus or platform via a network port 805 (also described in FIG. 11A), or the computer may be a part of the genotyping apparatus, or the data may be input by loading of removable media 811 (also described in FIG. 11A). The genetic data may be stored on removable media such as a removable disk 811 or non-removable media such as a hard disk drive or a solid state disk drive 803 (also described in FIG. 11A). The data and or results may be displayed at any time on a computer display 807 (also described in FIG. 11A) such as a monitor and may also be stored or printed at any time in the form of a genetic report. In step 908, genetic variants associated with phenotypes are obtained from scientific literature and sent to a computer system 800. The alleles or genotypes of each genetic variation or polymorphism identified from the genetic sample are then reviewed to determine whether the presence or absence of a particular allele or genotype is associated with a phenotype of interest.

The genotype variants and results from the biological samples are sent to, stored, and analyzed by a computer system (or digital device) 800, which produces a report of the results and analyses of the genomic data. The results and analyses may be accessed online by subscribers or their health care managers via an online portal or website as in step 910. The results and analyses can be viewed online, saved on a subscriber's computer, printed, or have mailed to the subscriber or health care manager 912. The individual may obtain genetic counseling or present the reports to physicians and other health professionals for personalized health management 914.

Genetic data, such as raw genotype code or data during the analysis process, such as during the analysis of the raw genotype code and during its correlation with phenotypes, or analyzed data, such as that which may appear or does appear within the genetic report, may also be displayable through virtual reality (VR) technology. The user who is viewing the genetic data through VR may be able to manipulate and change the genetic data, the genetic analysis, or any of the analytical processes, while being within the VR environment, either through the use of keyboard, control pad, mouse, wand or other pointing device, input device, audio and/or phonetic device(s), eye sensor that tracks the movement of the user's eye(s), tactile glove(s), or tactile suit. The VR environment may be viewable on a computer screen, or through the use of goggles, eye lenses or other optics, a helmet or other headpiece, or a VR room or other enclosed space, such as a VR machine that is large enough for one or more individuals to enter, either partially or fully. A Predictive Medicine Database, or other database containing genomic information, may also be viewable and/or modifiable via VR, as described above. Methods and apparatus for manipulating data with VR technology in general are known in the art and are described for example in US Patent Application Publication No. 20030033150, herein incorporated by reference in its entirety.

The computer system can also have a database of oligonucleotides sequences as described herein. For example, the computer system can have a database with at least 5, 10, 15, 20, 25, 50, 75, 100, 150, 200, 250, 500, 1000, 5000, 10000, 15000, 20000, 30000, 40000 or more oligonucleotide sequences and each of the sequences are associated with a genetic variant, such as a polymorphism. The database may have a variety of optional components that, for example, provide more information about the phenotypes. In some embodiments there is provided a computer readable medium encoded with computer executable software that includes instructions for a computer to execute functions associated with the identified genetic variants. Such computer system may include any combination of such codes or computer executable software, depending upon the types of evaluations desired to be completed. The computer system may also have code for linking each of the sequences to at least one phenotype, such as a condition, for example, a medical condition. Each medical condition in turn can be linked to at least one recommendation by a medical specialist and code for generating a report comprising the recommendation. The system can have code for calculating one or more scores for a phenotype, such as a condition or trait, for an individual, one or more action scores, one or more predictive medicine risk scores or carrier status or both, one or more organ system scores, or an overall genetic health score. The system can further comprise code for linking each of the sequences to at least one citation for a published journal article, such as a peer-reviewed journal article, showing the correlation between the genetic variation associated with the sequence to a phenotype, such as a condition or trait. The system can also have code for conducting genetic analysis based on specific panel(s) chosen. The system can also have code for one or more of the following: conducting, analyzing, organizing or reporting the results of reflex testing, as described herein. The system can also have code for generating a report. Different types of reports can be generated, for example, reports based on the level of detail an individual may want or have paid for. For example, an individual may have ordered analysis for a single phenotype, such as a condition, and thus a report may comprise the results for that single phenotype, such as a condition. Another individual may have requested a genetic profile for a panel or an organ system, or another individual may have requested a comprehensive genetic profile that includes analysis of all clinically relevant genetic variants with full reflex testing. The reports for each of the individuals can represent each of their requests.

The analysis generated can be reviewed and further analyzed by a GC and/or a medical professional such as a managing doctor or licensed physician, or other third party, in “Post-test”, for example as shown in FIG. 1, 128. The GC or medical professional or both, or other third party, can meet with the individual to discuss the results, analysis, and the genetic report (such as shown in FIG. 12A-G). Discussions can include information about the genetic variant(s), such as the genetic variant(s) (for example, polymorphism(s)) that is or are detected, how they can be inherited or transmitted (for example using the pedigree generated from the questionnaire), the prevalence of the genetic variant, prevalence or incidence of the phenotype, and information about the phenotype (for example, specific conditions or traits, such as medically or clinically relevant conditions), such as how the phenotype may affect the individual, results of reflex testing (as described herein), and if adverse conditions, preventative measures are associated with the genetic variants identified and analyzed or the phenotype(s) identified and analyzed or both. The GC or medical professional may incorporate other information, such as other genetic information or information from questionnaires in their analysis and discussion with the individual. Information about the phenotype, such as condition or trait, can include recommendations, such as follow-up suggestions such as further genetic counseling (FIG. 1) (130) or predictive medicine recommendations or preventive medicine recommendations for the individual's personal physician or other healthcare provider (132). Screening information, such as methods of breast cancer screening, may be discussed for example if an individual was found to be at a higher risk of breast cancer. Other topics that may be discussed include lifestyle modifications and medications. For example, lifestyle modifications may be suggested such as dietary changes and specific diet plans may be recommended or an exercise regimen may be suggested and specific exercise facilities or trainers may be referred to the individual. Common misconceptions may also be included, allowing the individual to be aware of preventive measures or other interventions that may be thought of as being helpful or useful but that have been shown in published literature to either not be beneficial or to actually be harmful. Alternative therapies may also be included, such as alternative medicines, such as fish oil supplements, or alternative therapies, such as acupuncture or yoga. Family planning options may also be included, as well as monitoring options, such as such as screening exams or laboratory tests that may detect or help monitor for the presence of a phenotype, or the progression of a phenotype. Medications that may prevent, limit the onset or delay the progression of a phenotype, such as a disease, the person is predisposed to, or a medication with high efficacy and low side effects may be suggested for an individual, or medications or classes of medications that an individual should avoid due to possibility of adverse reaction(s). For example, the medical professional may make an assessment of the individual's likely drug response including metabolism, efficacy and/or safety. The medical professional can also discuss therapeutic treatments, such as prophylactic treatments and monitoring (such as doctor visits and exams, radiologic exams, self exams, or laboratory tests) for potential need of treatment or effects of treatment based on information from the individual's genetic profile either alone or in combination with information about the individual's environmental factors (such as lifestyle, habits, diagnosed medical conditions, current medications, and others). Additional resources may also be listed, such as including information for the individual or the individual's physician or other healthcare professional to acquire additional information about the phenotype or the genetic variant(s) or both, such as links to websites that contain information on the phenotype, such as an internal website from the company that produces the genetic report or external websites, such as national organizations for the phenotype. Additional resources may also include reference to telephone numbers, books, or people that the individual may seek out to acquire more information about the phenotype or the genetic variant(s) or both.

A report with the results and analysis, can include information such as shown in FIG. 6, and be given to the individual during the consultation. Alternatively, the report, or a genetic report is depicted in FIG. 12. Reports may or may not typically also include a recommendation option by a physician or other licensed medical professional. In some embodiments, an individual may choose to further consult with a medical specialist or be referred to a medical specialist. Another report specific for the individual's physician (such as a ‘Healthcare Professional Summary’) can also be generated and given to the individual or the individual's physician such as depicted in FIG. 12E. The report can contain a patient summary, recommendations that may include follow-up recommendations, screening information, lifestyle modifications, alternative therapies or interventions, common misconceptions, monitoring information, family planning information, references to additional resources, medications, organ system scores(s), overall genetic health score, and a clinician summary. The recommendation options may be linked to a phenotype, such as a disease or trait, and can be presented in the report.

For example, an individual may have a disease for which they have either an increased or decreased risk of based on their genetic profile, and that disease is linked to a specific recommendation which is presented within the report generated for the individual. For example, an individual is found to have an increased risk of Macular Degeneration. Recommendations are offered to the individual concerning the need to avoid cigarette smoking (both first and second hand), to lose weight if they have a BMI greater than normal, to limit the amount of fat in their diet, to discuss the benefits of a Lipid Panel with their primary care physician or cardiologist (because high cholesterol is a modifiable risk factor for Macular Degeneration), and to monitor their blood pressure since high blood pressure is yet another modifiable risk factor. The recommendations may also state that the individual should become acquainted with an ophthalmologist so that a physician is aware of their predisposition to Macular Degeneration and can help monitor for disease manifestation and progression. Thus, the individual is encouraged to modify environmental and medical risk factors so as to attempt to either completely avoid or greatly minimize the risk of Macular Degeneration in the individual's future, or reduce morbidity associated with Macular Degeneration by allowing for appropriate therapies and treatment to be started as early as possible in the course of the disease (or so that diagnosis is made as quickly as possible once the disease or pre-disease process starts), or to delay the onset of Macular Degeneration. The recommendations also includes common misconceptions (such as caffeine consumption being shown to not decrease the risk of macular degeneration, as was once thought), lifestyle modifications (increasing intake of lutein and zeaxanthin by eating foods such as eggs and green vegetables), monitoring modalities (such as yearly eye exams by an ophthalmologist), and medications (prescription, over the counter, and alternative/herbal medicines) that have been shown to decrease the incidence of the disease or delay its onset (such as statins, lutein, β-carotene, vitamin C, vitamin E, and zinc, and omega-3 long-chain polyunsaturated fatty acids) (see for example, FIG. 12B). This type of information can be conveyed for each and every disease or trait that the individual has an increased risk for.

Reports may also contain other lifestyle information, such as lifestyle habits tailored to an individual, based on the individual's genetic profile, such as during the initial round of genetic testing or analysis or ascertained through reflex testing. For example, an individual may have his or her genetic profile determined at a health club or spa; and then, a genetically tailored fitness routine or workout based on that profile may be generated. An individual may also have a genetically tailored nutrition plan generated from his or her genetic profile. Other genetically tailored lifestyle habits, such as amount of sleep (or sleep cycle), type of recreational activity, type of relationships, type of work, hours of work, type of work, and many other lifestyle conditions may be tailored to the individual, based on his or her genetic profile. The lifestyle recommendations may also incorporate other factors, such as the gender, ethnicity, age, weight, lifestyle habits (smoking, drinking, sun exposure, stress levels, living environment, etc.), medications and alternative therapies such as herbs and supplements, family history of disease and/or personal history of disease (both past and current) of the individual.

Thus, the report for an individual's genetic profile, or Genetic Report can contain information about an individual's genotypes or phenotypes or both, as well as information directly related to that genotype or phenotype concerning preventive medicine recommendation options or intervention options or both (for example, FIG. 12A-G). The analysis of the genotypic and phenotypic data can include linked analysis and inclusion in the Genetic Report of all pertinent preventive medicine recommendation options or intervention options or both. Because many phenotypes, such as common diseases, are multifactorial, adjusting certain key environmental factors (such as lifestyle modifications) may help to decrease the risk and incidence of the phenotype, such as disease or condition, even in those people found to have a genetic predisposition for that phenotype, such as disease or condition (for example, FIG. 24). The genetic analysis process and the Genetic Report may link a phenotype, such as disease, risk with preventive measure options.

In one aspect, Preventive Measures (PMs) based on Preventive Medicine Recommendations or Interventions (PMRI's) are included in the Genetic Reports (for example, as described in Example 8). PMs based on PMRIs can be ascertained through a review of all current literature and both published and non-published studies concerning preventive measures that are shown to decrease the incidence or progression of the disease or both, or to delay time until disease onset (allow the individual to live longer without the disease or symptoms of the disease manifesting) or decrease the morbidity or mortality related to the disease, or both. PMs based on PMRI's may include, but not be limited to, information pertaining to one or more of following categories: Disease Education, Disease Warning Signs and Symptomatology, Lifestyle Modifications. Prescription Medications, Over-the-counter Medications, Monitoring Modalities, Vitamins, Herbs, or Alternative Treatments, Associated Diseases or Conditions, Current Treatments, Future Treatments, Being Connected to a Medical Professional, or Common Misconceptions. Some of the PMs based on PMRI's may also be linked to specific genetic variants that increase or decrease risk for a phenotype while others may be linked to the phenotype as a whole.

Two or more distinct Genetic Reports can be generated from the analysis of the same individual's genetic material. For example, one genetic report may be created for the patient and another genetic report may be created for the physician who ordered the test. Other targeted Genetic Reports may be created for the hospital, the insurance company, the government, or any healthcare provider (such as if a patient's primary care physician orders the genetic testing, a genetic report may be produced for the primary care physician while a targeted Genetic Report may also be created for the patient's Ophthalmologist and another targeted Genetic Report may be created for the individual's nutritionist). Information contained in the report can be written and targeted at the person who the report is specifically created for, such as described in Example 8.

The generation of Genetic Reports, or the genetic profiles for generating them, may be subject to different levels of service, such as for the reflex testing for ordered panel(s) or phenotype(s) or both. For instance, a low-cost service may be available whereas no reflex testing is available for any of the panel or phenotypes or both, a medium-cost service may be available where reflex testing goes only to round 2 and no further, and a high-cost service may be available where reflex testing goes through as many rounds as needed until no further reflex testing rounds exist. The panels themselves may also be differentiated by price, with certain panels that analyze a greater number of genetic variants or phenotypes, for both, for example, the Executive Panel Alpha, incurring an additional or higher fee compared with other panels that analyze a lesser number of phenotypes, for example, the Malaria Panel. OP-CADI also may be a different level of service that may have an additional or different fee associated with it.

In some embodiments, the level of service may be changed or altered at any time, and may incur an additional fee. For example, an individual may originally have selected the low-cost service but after reviewing their Genetic Report, they order another analysis to be conducted and Genetic Report to be compiled utilizing the same exact genotype data, or by supplying new genetic material for genetic testing and new genotyping, but this time with a high-level of service that gives results about all possible reflex testing relating to the panel or phenotypes they ordered, or both.

An individual with a pending genetic profile or genetic report to be generated may choose at any later date to have one or more of the following added to their analysis: reflex testing, additional panels, phenotypes, genes, specific genetic variant(s), or updated analysis based on updated or more recent research and genetic variant-phenotype association data. This additional genetic analysis and Genetic Report generation may require an additional fee. This additional analysis and report generation may be accomplished by utilizing the individual's raw genotype data from the original analysis or by ascertaining new genotype data by running either a stored biologic specimen (such as saliva, blood, tissue, hair, buccal tissue, such as from a buccal swab, purified DNA, etc.) or a new biologic specimen from the same individual through the genetic testing process at the laboratory.

The following examples illustrate and explain the present invention. The scope of the present invention is not limited by these examples.

### EXAMPLES

### Example 1

### Prophetic Example

### Genetic Profile for a Male Individual

A healthy male individual fills out a short, five minute ‘Presymptomatic Genetic Testing Questionnaire’ in person. The questionnaire includes questions on his current medical history, his family history including any known diseases, and any medications he is currently taking. There are also questions concerning his daily habits (such as tobacco, alcohol, caffeine, and drug use) along with his current exercise regimen. The completed questionnaire is reviewed by the presymptomatic genetic counselor (GC) who also may construct a genetic pedigree based on the male individual's (proband's) family history (FIG. 2). The GC briefly reviews his past medical and family history and gives the individual a copy of his genetic pedigree analysis that has already been conducted on his behalf. The GC tells the individual more in-depth information regarding further genetic testing services, different genetic testing options and panels available and, based on the individual's background, the GC recommends the Platinum Executive Package, also known as the Executive Panel Alpha, which analyzes thousands of genes and possible disease predispositions. The proband and the GC also briefly discuss the different cut-off and threshold values to be used during his genetic analysis, and the proband decides that he wants to be told about genetic variant-phenotype associations that may not be fully replicated yet, so the GVP score cut-off is set at greater than or equal to 0.75. The individual agrees on the Platinum Executive Package and signs a legal waiver. The GC takes a cheek swab sample and gives the individual a Confidential Client Number (CCN). He is shown that this corresponds to the client number printed on his cheek swab samples. The GC explains that his genetic data is never linked with his name or any other identifiable information except for his Confidential Client Number (CCN), in order to ensure the highest level of confidentiality. The GC gives the client's name and the corresponding CCN to the Managing Doctor who keeps a single copy of this encrypted information linking the CCN with the client's name in a fire-safe vault that is not kept online.

A follow-up appointment is scheduled with the same presymptomatic GC. The sample is sent to the lab by overnight courier and after the processing time to conduct the genetic material purification and genetic testing, the lab electronically transmits back to the central location and the results and recommendations based on the client's complete genetic profile are generated. The recommendations are included in an enhanced report, also known as the genetic report, is printed out and reviewed by the individual's GC. A managing doctor may also review and sign the report and may also discuss the results with the GC. The report includes information on the relevant genetic variants and their phenotypes that have been detected, including: ADORA2A, KALRN, 8q24, VKORC1, IRF5, and LRP6.

1) Polymorphism in ADORA2A gene detected. This specific polymorphism has been shown to greatly increase a person's sensitivity to caffeine. Increased caffeine sensitivity has been shown (specifically for this polymorphism) to correlate with reduced sleep quality and an increased risk of insomnia. Because of this, the following recommendations are made:

Individual has a genetic change that makes his body more sensitive to caffeine and that caffeine intake, even in the morning, may be affecting his sleep quality at night. Because of this, the individual may be advised to decrease or completely avoid products containing caffeine. He may also be advised that caffeine can be a difficult substance to stop using but that if he decreases his intake over a few weeks, he should be able to wean himself off it completely. Doing so may actually make him feel more awake and alert in the long run, since his sleep quality should improve. Based on the reflex testing conducting, he is also found to not be genetically predisposed to habitual caffeine use.

2) Polymorphism in KALRN gene detected. This specific polymorphism is thought to account for over 12% of all early-onset coronary artery disease for Caucasians. Because of this, the following recommendations are made:

Individual has more than a 100 times greater likelihood of having early-onset coronary artery disease than the general population's risk of early-onset coronary artery disease. Due to this increased risk, the individual may consider instituting numerous lifestyle modifications. His diet may be modified to limit his intake of trans-fats (such as hydrogenated oils that exist in fried foods, cakes, cookies, and margarine). The individual may also increase his exercise regimen, for example one option is that he exercises at least three times/week for at least 20 minutes per session.

The increased likelihood of early onset coronary artery disease also should be discussed with Pete's primary care physician. Increased surveillance may help monitor disease onset and progression, such as yearly cholesterol blood tests. Pete is advised to discuss with his physician other possible methods to screen for plaque build-up in his coronary arteries, such as Echocardiograms and/or Nuclear Medicine Stress Testing (such as Adenosine-Thallium Scans).

The individual is also advised to discuss with his primary care physician the benefits and disadvantages of once a day low dose Aspirin therapy. Aspirin therapy may help decrease the risk of heart attacks and in the individual's future. (If the individual is interested in making an appointment with a local Cardiologist, the GC or report may provide him with a referral or list of cardiologists in his community.)

3) Polymorphism in locus 8q24 detected. This polymorphism is associated with an increased risk for prostate cancer.

Due to individual's family history of prostate cancer along with a genetic polymorphism that is strongly associated with prostate cancer, the individual may be genetically predisposed to prostate cancer. This result should be discussed with his primary care physician. The importance of yearly screening exams for prostate cancer are made clear to the individual and he is instructed that he should always make sure to go for his annual exam and to make sure his prostate is always checked for possible warning signs of cancer or precancer. With proper screening, the impact of prostate cancer may be limited in his future, if it should develop.

Alternatively, the individual may also want to discuss this finding with an Urologist, who may advise for more radical screening modalities, such as yearly blood tests and possible radiological imaging of the prostate.

The individual is advised that now that he is aware of this predisposition, he can be empowered over it by making sure to stay connected with a healthcare professional that may screen him regularly for this disease so that they can take prompt action if it should ever manifest.

4) Polymorphism in VKORC1 detected (homozygous). This specific polymorphism has been shown to be associated with sensitivity for a frequently used blood-thinning medication called warfarin (Coumadin).

Warfarin is an oral medication frequently used in order to thin a person's blood to either avoid or treat a history of blood clots. The individual is advised that it is very important to discuss this with his primary care physician and that he should also consider adding this information to his official medical record. If he is given warfarin at the usual dosage, then he is at a much greater risk of bleeding complications. As long as this issue is known, however, then physicians may be able to take steps to avoid any potentially harmful effects of this medication if prescribing it to the proband ever becomes necessary. For example, his physician may start the medication at a lower dose and titrate up carefully, making sure to monitor him closely to make sure his blood does not become too thin. Recent studies have also shown that concomitant application of low dose vitamin K may also significantly reduce intra-individual warfarin dose variation and the individual's physician should also consider this as an option if the proband is ever required to take Coumadin.

5) Multiple polymorphisms in IRF5 detected. This specific group of polymorphisms has been shown to confer protection against Systemic Lupus Erythematosus (Lupus).

The individual is told about Lupus and that changes to his genetic code actually have been shown to protect him against Lupus. Because of this, he is 24% less likely to get Lupus than the general population's risk for Lupus.

6) Polymorphisms in LRP6 found along with no APOE4 polymorphisms detected. These genetic polymorphisms, along with absence of APOE4, have been shown to protect against Alzheimer Disease.

The individual is told that due to changes in one of his genes, he is actually protected against Alzheimer Disease. His likelihood of getting Alzheimer Disease is considerably lower than that of the general population.

The GC and the individual finish reviewing the rest of the findings. The individual's GC and the managing physician then review these six findings discussed above, along with the other findings, with the individual and discuss some of the many genes that he did not have any polymorphisms in. The individual is advised that his family members may also benefit from genetic testing because they may contain the same polymorphisms that were detected in the individual or they may contain different genetic variants or different combinations of variants that may put them at risk for different diseases.

These recommendations are handed to the individual in a confidential envelope as an Enhanced Client Report, also known at a Genetic Report, and an Enhanced Physician's Report, also known as the Healthcare Professional Summary, for the individual to give, at his discretion, to his physician(s).

### Example 2

### Prophetic Example

### Determining Predispositions and Risks to Future Children

A male and female, referred to as ‘the couple’, is interested in genetic testing to determine diseases or conditions they may pass on their future children. The couple fills out a Carrier Questionnaire. The questionnaire has questions on the medical history as well as the family histories and any known genetic disorders. The questionnaire also asks about any specific diseases that the couple is worried about. The Carrier GC reviews both of their completed questionnaires and writes a brief pre-meeting note, and may complete a genetic pedigree.

The GC discusses their medical history and reviews their genetic pedigrees, discusses what can and cannot be tested and discusses the limitations of genetic testing as well as the potential insights that may be learned. The GC also talks about some of the most prevalent diseases that are screened for in the various panels, such as Cystic Fibrosis and Type II Diabetes, and some possible implications if the genetic testing is positive or negative for these diseases. The GC explains tests for hundreds of rare genetic disorders are available all at once and provides information about these as well. The GC also explains that even though the couple is primarily interested in diseases and traits that may affect their future children, they may also find out potentially important information about themselves since it is their genetic codes that are being analyzed. The couple agrees to the service and chooses the ‘Complete Carrier Package’, also known as the Carrier Screening Panel, that investigates hundreds of diseases and traits that the couple could potentially pass on to their child. The GC also explains and reiterates that, at times, carrier genetic testing may also detect disease predispositions that may affect the couple directly and that it is important that they understand this. The couple agrees and may sign a legal waiver and the GC then takes three separate cheek swabs each from the male and female and gives them their respective Confidential Client Numbers, explaining how this is an added safeguard to protect their confidential genetic information so that not even the laboratory may have access to their names. The couple also discusses the cut-off values and their meaning with the GC, and chooses to have all genetic variant-phenotype associations analyzed with a GVP score equal to or greater than 0.5.

The couple schedules a follow-up appointment in a few days with their same GC. The cheek swab specimens are packaged and shipped overnight to the lab. A few days later the couple's genotype results are transmitted back to the central location and the results are further processed and analyzed to produce a report for their GC, an enhanced report (the Genetic Report) for the couple, and another enhanced report for their physicians (the Healthcare Professional Summary). The GC and the managing physician may review and discuss the reports.

The report includes the relevant genetic variants and their phenotype associations that were detected, including for the male: genetic variants in the genes HFE, MTTL1, BMP2, and RYR1, and for the female: genetic variants in the genes HFE, MTTL1, BMP2, MTHFD1. The couple meets with their same GC and the managing physician. The GC tells them that they are both carriers of a genetic polymorphism in their HFE gene and that, if they each pass on these genetic variants to their child, the child may have the disease Hemochromatosis. This is an iron-storage disease and can lead to problems with the pancreas, severe liver disease and also liver cancer if not detected and treated properly. The GC explains that because they each have one Hemochromatosis gene, their child has a 25% chance of being likely affected by the disease (which requires both copies of the gene to be present) or a 50% chance of being a carrier or a 25% chance of being neither a carrier nor affected. During the explanation, the GC utilizes a Punnett Square so that the couple can visualize the information (FIG. 3). The GC goes on to explain that Hemochromatosis actually has a varied degree of expressivity and penetrance and that even if their child has both abnormal genes, the disease itself may never manifest or, if it does, it may not be detrimental enough to cause any noticeable disease. However, reflex testing determines that they both contain polymorphisms in two other genes, MTTL1 and BMP2, which have both been shown to increase the risk of Hemochromatosis manifesting as a serious disease in people who have polymorphisms in the HFE gene, as they do. Therefore, the risk of Hemochromatosis is a possibility for their future offspring.

Hemochromatosis is a recessive disease, which means that both copies of the gene must be mutated in order for the disease to manifest. The normal gene is usually represented by a capital “H” and the mutated gene, the gene that causes Hemochromatosis, is usually represented by a lower case “h”. However, this Punnett Square has been simplified so that the normal gene is represented by the words “Normal Gene”, also may be represented as “Normal Allele” and the Hemochromatosis gene is represented by words “Disease Gene”, also may be represented as “Disease Allele” in FIG. 3.

As can be visualized by the Punnett Square, for a recessive disease to manifest, one disease gene has to be contributed by the mother and the other disease gene has to be contributed by the father (bottom right square). This shows that there is a 25% chance of their child likely having the disease and a 75% chance of not having the disease (that can be further broken down into the child having a 25% chance of being a non-carrier and 50% chance of being a carrier). These statistics hold for each and every child they have, so there is always a 25% chance of their child likely having Hemochromatosis. This assumes a 100% expressivity and full penetrance, meaning that if the child has both diseased genes (is homozygous for the polymorphism), then the disease always manifests. As explained above in the example and as the genetic counselor would explain to the client, this is not entirely true for many genetic diseases, including Hemochromatosis. The genetic counselor is specially trained in how to properly convey this information to the layperson.

The GC states that their HFE genetic polymorphism is actually one of the most common human polymorphisms and occurs with a carrier frequency of around 10% of the Caucasian population and a disease prevalence of approximately 1 in 300 people in the US (Merryweather-Clarke, A. T., J Med Genet. 1997 April; 34(4): 275-278 Dr. Hady Sfeir, “Hemochromatosis”, eMedicine Article, www.emedicine.com/MED/topic975.htm, 6/2005). As long as physicians know that a patient has the disease-causing Hemochromatosis polymorphisms, the person can be easily monitored and, if disease manifests, treated by scheduled blood draws in order to decrease the overall iron content of blood.

The GC recommends that the couple review information available about Hemochromatosis and gives them information about and the web address of the American Hemochromatosis Society (www.americanhs.org). The GC states that, many times people with these genetic variants never experience any noticeable disease but, if they do, as long as it is treated, people with Hemochromatosis can lead a normal life and have a normal life span. Because of this, the GC may recommend that the couple pursue a routine pregnancy (instead of other fertility options, which include in-vitro fertilization (IVF) with pre-implantation genetic diagnosis (PGD), having an egg or sperm donor, or adoption). If the disease were more serious, such as juvenile onset macular degeneration (which causes irreversible blindness in children), then the GC may have talked more about other options, such as IVF with PGD, which allows for the embryo fertilized in-vitro to be screened for the disease before it is implanted into the uterus.

The male individual of the couple also has a polymorphism in his RYR1 gene, which means that he is very likely affected with malignant hyperthermia and that their children each have a 50% chance of also having this polymorphism and disease. Malignant hyperthermia is a very serious disease that is triggered by general anesthesia. Therefore, it is important that the male individual inform his primary care physician that he is genetically predisposed to malignant hyperthermia so that this information can be added to his permanent medical record. He is also advised to always inform the surgeon and anesthesiologist of this predisposition if he ever needs surgery for any reason. While malignant hyperthermia can be extremely serious when it manifests, steps can be taken to limit its consequences and avoid serious injury or death as long as the anesthesiologist is made of this predisposition. The vast majority of people with this disorder live normal lives and therefore the GC recommends that any children they have get tested for this genetic polymorphisms so that they, too, know if they are predisposed to (or are likely affected by) malignant hyperthermia. The couple is also advised that if, for any reason, their child needs surgery before genetic testing can be conducted, they should inform the anesthesiologist that the father is genetically predisposed to malignant hyperthermia and that the child may be as well. Lastly, the GC explains that his primary care physician may be interested in ordering a simple blood test to measure serum creatine kinase levels, which has shown to be elevated in people with this specific polymorphism. All of this information is included in a report that he can give to his physician.

The female individual is Irish Caucasian and is found to have a homozygous polymorphism in her MTHFD1 gene that has been associated with both an increased risk of having severe abruptio placentae (in Irish populations) and also with an increased risk of having a child with neural tube defects (in Italian and Irish populations). The managing physician explains what abruptio placentae is and how it may threaten a pregnancy. Her risk of having abruptio placentae is significantly elevated over that of the general population and therefore this is a very important predisposition that her obstetrician may find important and may need to know about. As long as the obstetrician knows to check for this and to educate her about the potential warning signs, then they may be able to institute measures that will actually avoid pregnancy loss if this should occur. Also, due to her having a significantly increased risk over the general population's risk of having a child with a neural tube defect, such as spina bifida, it may be very important that she begins taking daily prenatal vitamins even before she becomes pregnant, since the vitamins are most effective during the first few weeks immediately after conception. Her obstetrician may also want to be made aware of her increased risk of having a child with neural tube defects since many times this can be detected very early in the pregnancy through the use of modern ultra-sound machines and maternal blood tests.

The genetic counselor continues to review all the other significant findings and also goes over a condensed list of the multitude of genes that did not contain any polymorphisms and they also discuss some of the diseases that they are actually protected against, and that their children may most likely be protected against as well. For instance, the GC tells them that the male individual's genes contain a genetic change that protects him against Alzheimer Disease and that the female's genes contain genetic polymorphisms that protect her against obesity and diabetes.

The GC gives the enhanced genetic reports to the couple, making sure to give them a copy that they can give to their primary care physician and their obstetrician. The GC tells them that if they would like further genetic counseling, they can schedule that now or anytime in the future.

### Example 3

### Prophetic

### Genetic Profiles for Children

A mother has two children, a boy aged five and a girl aged two. The mother is interested in a special genetic testing panel specifically for children that tests not only for diseases but also certain conditions that may influence a child's learning and development. The mother schedules an appointment for herself and her two children with a Pediatric Genetic Counselor (GC). The Pediatric GC explains to the mother the pediatric genetic testing panel can detect diseases that the children could possibly be afflicted with either now as children or later-on as adults, and also detect disorders that could impede the children's ability to learn and develop properly. For instance, if a gene for obesity is discovered then preemptive lifestyle modifications such as diet, exercise, and parental oversight may greatly limit the predisposition to gain significant weight. Alternatively, knowing that someone has a genetic predisposition for obesity may actually help that individual come to terms with their weight because it isn't fully in their control. Another example discussed is a gene that controls medication metabolism and what would happen if it is abnormal. In this case, the child may not properly metabolize some medications, which could then cause very high levels of that medication in the blood and this could lead to adverse drug reactions and serious complications.

After further discussion about what genes are tested for and some of the possible consequences of finding an abnormal gene, the mother agrees to the genetic testing and chooses to have the “Complete Pediatrics Panel”, also known as Pediatric Panel Alpha, run on both of her children. The GC discusses with the mother the different cut-off value options available as well as their meaning, and the mother chooses for a GVP score equal to or greater than 0.5. The GC discusses the implications of allowing genetic variant-phenotype associations with lower GVP scores to be included in the analysis and the mother states that she understands and wants to proceed. The GC then takes cheek swabs of both children and gives the mother their Confidential Client Numbers.

The mother pays the fee for each child and schedules a follow-up appointment. The GC packages the cheek swab specimens and sends them to the lab. One week later, the results are transmitted electronically and are reviewed by the GC and the managing doctor. Some of the relevant results include genetic variants, such as polymorphisms, in the genes HCRT, ACTN3, HLA-C*0602, and TIRAP detected in the daughter, and the polymorphisms CFTR, MC1 R, and DRD4 detected in the son.

The mother returns and meets with the same GC and the managing doctor. The GC first reviews the daughter's genetic profile and states that she only has a few non-serious genetic changes.

1) Polymorphism in the HCRT gene. Predisposition to Narcolepsy

The first is a polymorphism in her HCRT gene, which means that she may be predisposed to narcolepsy. The GC explains the symptoms of narcolepsy include excessive sleepiness and possibly falling asleep in situations where most people would remain awake (such as while in class or when driving a car). Because of this, the mother should be aware that if her daughter is having difficulty in school or ever shows signs of excessive sleepiness, this could actually be a treatable medical condition and that the daughter should then see a sleep specialist (the GC can refer them to or may give them a list of qualified sleep medicine physicians in their community, if necessary).

2) Polymorphism in the ACTN3 gene. This specific genetic polymorphism is associated with elite athletic performance.

The daughter possesses a genetic polymorphism in the ACTN3 gene that has been associated with exceptional athletic performance. This specific genetic polymorphism promotes much more efficient aerobic muscle metabolism that may allow the daughter to perform physical activities, such as running and swimming, for much longer continuous periods of time (estimated at 33% in some studies) without reaching exhaustion. This polymorphism has been found to be overrepresented in endurance athletes around the world. Because of this, the mother may want to encourage her daughter to participate in endurance-related sports and athletic programs both at school and during the summer.

3) HLA-C*0602 detected. Predisposition to Early-onset Psoriasis.

The GC explains that the daughter contains a specific form of HLA-C, known as *0602, and that this genetic marker has been associated with a very significant increased risk of early-onset psoriasis. This genetic predisposition is important for the mother to be aware of since psoriasis is a condition that is usually treatable by a dermatologist. Her daughter can receive the proper care right at the onset if the disease manifests, rather than at a later time when emotional and psychological stress may have had a significant impact upon the child. The dermatologist may also be more likely to treat the problem more aggressively right at the onset since her daughter is known to be genetically predisposed to it.

The managing doctor describes the symptoms of psoriasis and states that there are many different types of treatments available if the disease should manifest.

4) Polymorphism in the TIRAP gene. This polymorphism may be associated with protection against serious infectious diseases.

The daughter has a polymorphism in her TIRAP gene. This polymorphism has recently been shown to confer protection against infectious diseases, such as invasive pneumococcal disease. Because of this, she may actually find that she is more resistant to serious infections than most people are.

The GC and the managing doctor also discuss the son's results with the mother.

1) Polymorphism in the CFTR gene.

The son is a carrier of a cystic fibrosis genetic polymorphism. However, he most likely has one normal gene since only a single genetic polymorphism was detected, so he will most likely not be affected by this disease.

The fact that he is a carrier of a CFTR genetic polymorphism may be important information for the son to know about when he does decide to marry. When he does decide to start a family, it may be prudent for his wife to also have her CFTR gene tested, if it hasn't been already, in order to ascertain the true risk associated with having a child with cystic fibrosis, which is a very serious illness. By knowing that the son is a carrier of this genetic mutation, he can now be empowered over it potentially creating disease in future generations.

2) Polymorphism in the MC1R gene. This specific polymorphism has been associated with red-hair, fair-skin, increased risk of melanoma, and also increased anesthesia requirements during surgical procedures.

The GC notes the son has red-hair and fair-skin and these traits are mostly likely due to a genetic change that was detected in his MC1R gene. Besides the hair and skin color traits, this genetic change also makes him extra-sensitive to the harmful UV-rays of the sun and increases his risk of developing melanoma. While this may not affect him for many years, melanoma is very deadly and it is important that both his primary care physicians and his dermatologists always know about this predisposition. Because of this, an optional recommendation presented to the mother is that he become acquainted with a dermatologist and receive full-body checks for melanoma on a regular basis. Hopefully, with the knowledge of this predisposition and increased monitoring, any melanoma that develops may be detected and removed from his body very quickly. Increased monitoring due to a known genetic predisposition has the potential to greatly limit the morbidity and high mortality of this disease. If his predisposition to melanoma was not known, then a suspicious lesion may have been discovered either months or years after it developed, which may then necessitate radical surgery with resultant physical deformity and also a high likelihood of metastatic disease (even after surgical removal) and possibly death.

The same genetic change in the MC1R gene that predisposes the son to melanoma has also been shown to correlate with increased anesthesia requirements during surgery. If he ever has to have surgery, this may be important information to discuss with the surgeon and anesthesiologist so that they can make sure he receives the proper analgesia and sedation during the entire procedure.

2) Polymorphism in the DRD4 gene and other relevant genes detected and these polymorphisms may be associated with Attention-Deficit-Hyperactivity Disorder.

The GC notes that while the vast number of genes screened for were all normal, the son does have another change in his DRD4 gene, which may predispose to attention deficit hyperactivity disorder (ADHD). The GC describes the symptoms of this disease, stating that some children who have ADHD have significant learning difficulties until this disease is diagnosed and properly treated.

The GC states that if behavioral or learning issues arise, the son may benefit from seeing a psychiatrist who specializes in ADHD (and the GC can recommend the a list of names of child psychiatrists in her community, if needed). With proper oversight and treatment, it is very likely that the son may do just fine in school if it is found that his trouble concentrating and learning new material is due to this disorder.

The GC gives the mother a copy of the results and recommendations for both children, as well as referrals to a dermatologist and a pediatric psychiatrist. Additional copies of the reports are included for these doctors and also for the children's pediatrician. The mother is told that the GC would be happy to discuss these results with any of the doctors if they have any questions.

### Example 4

### Prophetic

### Genetic Profile for a Single Gene and Condition

An individual has intense pain with breastfeeding her child and wants to switch to formula. Before switching she wants to determine whether breastfeeding may increase her baby's IQ level. A single gene, such as the breastfeeding intelligence gene is tested for in the baby by having a managing physician or the child's pediatrician roll a small q-tip-like swab on the inside of the baby's cheek to obtain a genetic sample.

The baby is found to contain the gene that may increase her intelligence only if she is breastfed. The physician also states that they can refer the mother to a nurse who specializes in breastfeeding and may be able to assist with some techniques to make it less painful, if the mother decides to continue with breastfeeding.

### Example 5

### Prophetic

### Algorithm for Calculating Predictive Medicine Risk (PMR)

The following example utilizes the multiplicative model but any algorithm known in the art may also be utilized instead of the multiplicative model, such as the additive model.

The Greek letters α, β, and γ is used to represent different alleles. For example, these Greek letters may represent nucleotides (adenine, cytosine, guanine, or thymine), such as with single nucleotide polymorphisms where, for example, a may represent a cytosine, β may represent a thymine, and y may represent an adenine. Alternatively, the Greek letters α, β, and γ may represent alleles of any other type of genetic variant, such as insertions and deletions, for example where a may represent the insertion and β may represent the deletion, or for copy number variations, where a may represent 1 copy, β may represent 2 copies, and y may represent 3 copies. As can be seen, the Greek letters are used to represent the different possible alleles of any type of genetic variant, such as any type of mutations, SNPs, DIPs, CNVs, translocations, repeats, etc.

Many genetic variants are biallelic, meaning that there are two possible alleles (Allele α and Allele β) and therefore three possible genotypes: αα, αβ, ββ. At times, however, genetic variants may have more than two possible alleles. For example, triallelic genetic variants have three possible alleles (Allele α, Allele β, and Allele γ) and therefore six possible genotypes: αα, αβ, ββ, αγ, βγ, γγ. This can be expanded out to include as many alleles and genotype combinations as is necessary to capture all the possible variations at a specific genetic variant. Therefore, while many times there are usually just two alleles and three possible genotypes, there can also be three alleles and six possible genotypes, and there can also be more than three alleles and more than six possible genotypes.

The following example considers the scenario where two genetic variants and their genotypes have been detected through genetic testing that are associated with risk for phenotype X. While two genetic variants are utilized in this example to convey the application of the algorithm to multiple independent and relevant genetic variants associated with risk for the same phenotype, any number of genetic variants may be used and this algorithm is applicable to any number of genetic variants.

For this example, two genetic variants (A and B) are detected that are associated with phenotype X, and the genotypes for these two genetic variants are associated with risk for phenotype X.

Genetic variant A is a biallelic SNP with allele 1=α and allele 2=β
Genetic variant B is a biallelic SNP with allele 1=γ and allele 2=δ

In this example, results from genetic testing yield the following raw genotypic data for the genetic variants:

Genetic variant A genotype detected: αβ

Genetic variant B genotype detected: δδ

Note that α, β, γ, and δ are meant to represent different alleles and, as stated above, they can represent any type of allelic variant, such as a nucleotide, an insertion or deletion, a copy number variation, etc. Genetic variants A and B are distinct and represent separate genetic variants that are both associated with and relevant for phenotype X. For example, if genetic variants A and B are both SNPs, genetic variant A's α may represent a cytosine and genetic variant A's β may represent a thymine while genetic variant B's γ may represent a thymine and genetic variant B's 6 may represent a guanine. As stated above, the Greek letters are just meant to represent different possible alleles and each is unique to each specific genetic variant, as described. In the example given, genetic variant A having the αβ genotype means it is heterozygous for its two alleles and genetic variant B having the δδ genotype means it is homozygous for one of its alleles.

Genetic variant A genotype detected: αβ→risk value A_{αβ} for phenotype X
Genetic variant B genotype detected: δδ→risk value B_{δδ} for phenotype X

The risk value for each allele or genotype that is associated with each specific genetic variant-phenotype association is ascertained from published studies and published literature, such as journal articles.

### Risk Values: OR=Odds Ratio or RR=Relative Risk

If the risk values for the genetic variants alleles or genotypes (risk value A_{αβ} and risk value B_{δδ}) are given as RRs, then skip to step 2. If the risk value for either or both of the genetic variants and their alleles or genotypes are given as ORs, then proceed to step 1.

Step 1—Conversion

Genetic variant A ORs=(ORA_{αα}, ORA_{αβ}, ORA_{ββ})
Genetic variant B ORs=(ORB_{γγ}, ORB_{γδ}, ORB_{δδ})
Convert ORs to RRs, as described previously herein.
Genetic variant A RRs=(RRA_{αα}, RRA_{αβ}, RRA_{ββ})
Genetic variant B RRs=(RRB_{γγ}, RRB_{γδ}, RRB_{δδ})

Step 2—Assess allele or genotype frequencies from matched reference population for the allele or genotypes of genetic variants A and B

Data ascertained from resources as described, such as The International HapMap Project or the National Center for Biotechnology Information (NCBI)'s dbSNP.

Allele Frequency (AF) of genetic variant A=AFA_{α}, AFA_{β}
Allele Frequency (AF) of genetic variant B=AFB_{γ}, AFB_{δ}
Genotype Frequency (GF) of genetic variant A=GFA_{αα}, GFA_{αβ}, GFA_{ββ}
Genotype Frequency (GF) of genetic variant B=GFB_{γγ}, GFB_{γδ}, GFB_{δδ}

Step 3—Calculate Cumulative Generic Population Risk Load (GPL)

GPL for genetic variant A=GPL_{A}=((RRA_{αα}(GFA_{αα}))+((RRA_{αβ})×(GFA_{αβ}))+((RRA_{ββ})×(GFA_{ββ}))
GPL for genetic variant B=GPL_{B}=((RRB_{γγ})×(GFB_{γγ}))+((RRB_{γδ})×(GFB_{γδ}))+((RRB_{γδ})×(GFA_{δδ}))
Cumulative GPL for phenotype X for genetic variants A and B=GPL_{xc}=(GPL_{A})×(GPL_{B})

Step 4—Calculate the individual's Proband Risk Load (PRL)

The PRL is based on the relative risks associated with the specific detected allele or genotype for each genetic variant. In this example, the PRL is calculated from genetic variant A (detected genotype=αβ) and from genetic variant B (detected genotype=δδ

### PLR for Phenotype X=PRLₓ=(RRA_{αβ})×(RRB_{δδ})

Step 5—Calculate the Cumulative Genetic Risk (CGR)

### CGR for Phenotype X=CGRₓ=PRLₓ/GPL_{xc}

Step 6—Calculate the Predictive Medicine Risk (PMR)

The generic gender-specific population lifetime risk percent (GLR) for phenotype X (GLRx) is ascertained from literature and resources as previously described herein.

### PMR for Phenotype X=PMRₓ=(CGRₓ)×(GLRₓ)

(The upper bound for the PMR of multifactorial phenotypes may be set at an upper limit, such as 95%, as these phenotypes may not be fully determined by genetic factors alone. The lower bound for the PMR of multifactorial phenotypes may be set at a lower limit, such as 0.0001%, as these phenotypes may not be fully determined by genetic factors alone.)

### Example 6

### Prophetic

### Organ System Score and Overall Genetic Health Score

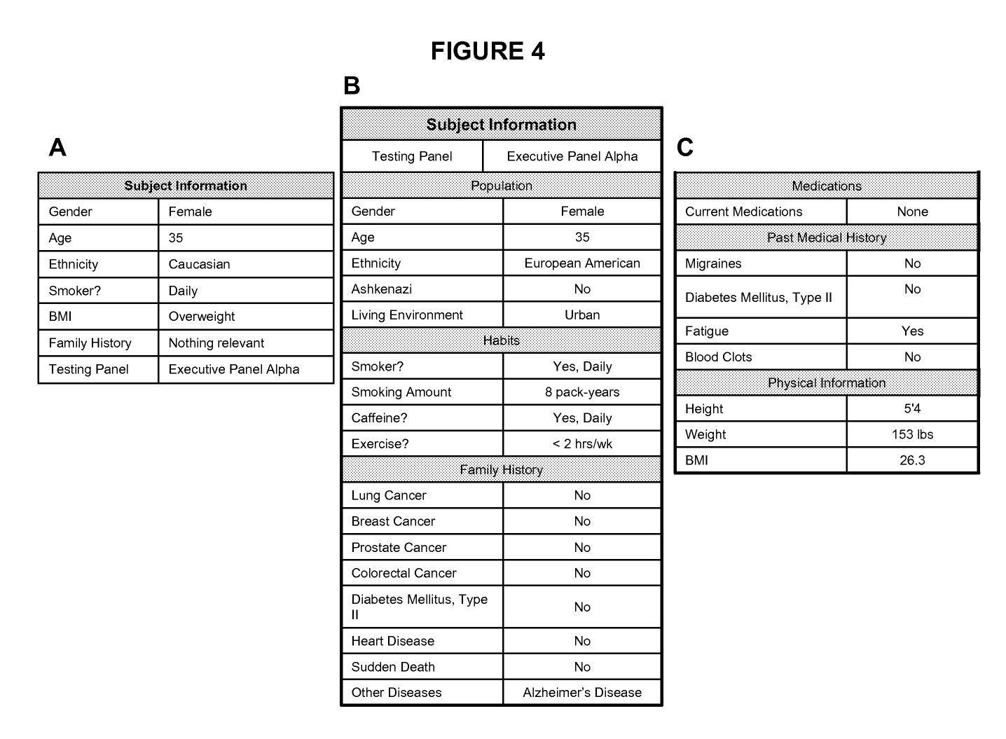

A 35 year old Caucasian female smoker (FIG. 4A), 20 lbs overweight for her age and height, presents for genetic testing and chooses the Executive Platinum Package, also known as the Executive Panel Alpha. Relevant information is obtained from the individual, entered into a system, and utilized during analysis. The information includes the individual's gender, age, premenopausal state, ethnicity, smoking habit, weight information, family history of no breast cancer, and testing panel request for the Executive Platinum Package of having thousands of clinical polymorphisms analyzed.

A raw polymorphism data chart for the individual is generated (for example, Table 13). The SNP-Disease Coefficient Rating (SDCR), also known as the GVP score, is obtained by analyzing studies determining the correlation between a genetic variant, such as a SNP, and a phenotype, such as a disease or trait. The Generic Absolute Risk (GAR), also known as the GLR, is determined from published literature while the Cumulative Genetic Risk (CGR) may include information such as Risk Values, expressed as an odds ratio (OR), relative risk (RR) or hazard ratio (Z). The Cumulative Genetic Risk (CGR) is determined by incorporating all the relevant SDCs (SNP-Disease Coefficients, also known as the GVP score) applicable to a specific disease or condition, based on selected cut-off threshold values. For example, for a disease X, polymorphisms A and B are detected, where A has an OR₁ and B has an OR₂. Utilizing HapMap data for the reference population (such as CEU HapMap data for a Caucasian or European American population), genotype frequencies for each of the two alleles (Allele 1 and Allele2) or each of the three genotype possibilities (Allele1/Allele1, Allele1/Allele2, and Allele2/Allele2), as there are usually three genotype possibilities, such as with biallelic SNPs, although there may be more genotype possibilities, such as with triallelic SNPs, as discussed in Example 5, for each of the genetic variants can be ascertained and these may then be multiplied by the risk value (RV), such as relative risks, for each of the respective allele or genotypes for that same genetic variant and specific phenotype, such as disease, association and these values (taking into account all the possible allele or genotypes, the frequency in the population for each allele or genotype added or multiplied, depending on whether an additive or multiplicative or other methodology is used, by the allele or genotype risk value for each of the genetic variant's alleles or possible genotypes) may then all be added together for each of the genetic variant to give the cumulative generic population risk load (GPL) for each genetic variant and then the GPLs for each other genetic variants (A and B) may be multiplied together to give the cumulative generic population risk load (GPL_{c}) while the CGR for disease X would be RV₁+RV₁ or RV₁×RV₂, such as OR₁+OR₂ or OR₁×OR₂ or RR₁+RR₂ or RR₁×RR₂, (depending on whether the additive or multiplicative or other methodology is used) divided by the GPL_{c}. If the prevalence of the phenotype is high (for example, greater than 10%), then the odds ratios may be converted to relative risks first and then either added together or multiplied together as before, again depending on whether the additive or multiplicative methodology is utilized. If no risk value (NRV) is available, a placeholder risk can be assigned by a physician or genetic counselor or bioinformatics specialist analyzing the ‘individual’s genetic profile. A Predictive Medicine Risk (PMR) can be determined by, in one iteration, relating back to the previous models multiplying the GAR, also referred to as the GLR, by the CGR or in another iteration methodology relating back as stated by adding the GAR, also referred to as the GLR, with the CGR depending on the methodology, GAR, prevalence, and incidence of the specific multifactorial phenotype, such as disease, that is being analyzed at that time. A Clinical Significance Rating (CSR) can also be determined, where 0 would indicate no clinical use, 1 would indicate limited clinical significance, value, or use, 2 would indicate moderate clinical significance, 3 would indicate very useful in a clinical setting, where a medical professional would likely find the result valuable, and 4 would indicate extreme clinical significance, possibly relating to a life-threatening condition. The DIR, or Disease and Trait Impact Rating, also referred to as the PIR, indicates the severity of a phenotype that is correlated with a genetic profile. For example, the DIR ranges from −3 to +3, where −3 causes sudden death or debilitating disease, −2 indicates a serious disease, a disease or condition that is difficult to cure, may cause death, or has significant negative life consequences, −1 indicates a condition that is usually manageable, 0 is a neutral condition or trait, +1 a slightly positive impact; +2 is a helpful trait, and +3 is a provides a significant advantage to the individual.

The CGR Multiplier and PMR (Predictive Medicine Risk) or NRV (No Risk Value) Multiplier is chosen (see for example FIG. 9), as is the CSR (Clinical Significance Rating) and the DIR (Disease and Trait Impact Rating also known as the Phenotype Impact Rating or PIR) and the NMF (Notice Me Factor). The Action Score (AS) may be determined by multiplying the SDCR, CGR Multiplier, PMR or NRV Multiplier, CSR and DIR or by multiplying the CSR, DIR, and NMF, or the CSR, PIR, and NMF. The Cumulative Action Score (CAS) may be determined by adding all the AS's for all of the phenotypes, such as diseases or traits, that fall under the same organ system, which may also be identified by medical specialty and then dividing by the total number of AS's, The Cumulative Action Score may therefore be the average action score for that organ system, which can also be identified by medical specialty. Each of the individual AS's is already weighted in terms of clinical significance, degree of phenotype benefit or harm, and significance of the change in risk, as previously discussed.

A chart for scores by organ system and an overall genetic health score can be used, such as shown in FIG. 10. The Cumulative Action Score (CAS) can be filled in for more than one organ system and determined for an organ system. The organ system score or Indicator of Genetic Health of an Organ System can be indicated by a color. Red would be used for scores less than −10, indicating highly important to discuss with client and may be highly important for client to follow-up with their physician or specialist based on this information, pink can be used for scores between −1 to −10 to indicate moderately important risk, green can be used for scores of 0 to indicate no pertinent deleterious or protective information discovered although organ system was accessed, blue can be used for scores between +1 to +10, to indicate moderately important protection, gold can be used for scores >+10 indicating very beneficial protection, and no color can be used for an Organ System or Medical Specialty if it was not accessed. The overall genetic health score can be determined, as described above, by adding all the CAS, which may be used as an indicator for genetic wellness and is also represented by a color as is the Indicator of Genetic Health of an Organ System, and dividing by the total number of CAS's, as previously described

Selected raw polymorphisms data is shown in Table 13.

TABLE 13
Selected Raw Polymorphism Data
Oncology & Women's Health

```
Polymorphism in LOC643714
  Locus 16q12
  SNP Identifier: rs3803662
  Genotype: TT
  Clinical Correlation: Breast Cancer, Estrogen-receptor Positive
  Odds Ratio: 1.64
    *SNP-Disease Coefficient =
  Population: Caucasian
Polymorphism in Intergenic Region
  Locus 2q35
  SNP Identifier: rs13387042
  Genotype: AA
  Clinical Correlation: Breast Cancer, Estrogen-receptor Positive
  Odds Ratio: 1.44
    SNP-Disease Coefficient =
  Population: Caucasian
Polymorphism in MAP3K1
  Locus 5q11.2
  SNP Identifier: rs889312
  Genotype: CC
  Clinical Correlation: Breast Cancer
  Odds Ratio: 1.27
    SNP-Disease Coefficient =
  Population: Caucasian
Polymorphism in TNRC9
  Locus 16q12.1
  SNP Identifier: rs3803662
  Genotype: TT
  Clinical Correlation: Breast Cancer
  Odds Ratio: 1.39
    SNP-Disease Coefficient =
  Population: Caucasian
Polymorphism in FGFR2
  Locus 10q26
  SNP Identifier: rs1219648
  Genotype: GG
  Clinical Correlation: Breast Cancer
  Odds Ratio: 1.64
    SNP-Disease Coefficient =
  Population: Caucasian, Post-menopausal Women
```

Pharmacology & Oncology

```
Polymorphism in CYP19A1
  Locus 15q21.1
          SNP Identifier: rs4646
          Genotype: CA
          Clinical Correlation: Improved Treatment Efficacy of Aromatase Inhibitor Letrozole
          in Advanced Breast Cancer, Estrogen-receptor Positive
          Hazard Ratio: 0.52
    SNP-Disease Coefficient =
          17.2 months to breast cancer disease progression with genotype CA or AA and
          Letrozole versus 6.4 months to disease progression with genotype CC and Letrozole
    SNP-Disease Coefficient =
          Population: Post-menopausal Women with Estrogen-receptor Positive Breast Cancer
        Polymorphism in MDM2
          Locus: 12q14.3-q15
          SNP Identifier: rs2279744
          Genotype: GG
          Clinical Correlation: Resistance to Topoisomerase II-Targeting Chemotherapeutic
          Drugs (Etoposide, Mitoxantrone, Amsacrine, and Ellipticine)
    SNP-Disease Coefficient =
        Polymorphisms not detected in RAD51 & XRCC3
          Loci: 15q15.1 & 14q32.3
          Haplotype Identifier: rs1801320 & rs861539
          Haplotype: G-T
          Clinical Correlation: This haplotype is associated with over a 700% increased risk of
          developing chemotherapy-related AML (Acute Myelogenous Leukemia), a type of
          blood cancer.
    SNP-Disease Coefficient =
```

Endocrinology & Traits

```
        Polymorphism in APOB
          SNP Identifier: rs679899
          Locus: 2p24
          Genotype: CC
          Clinical Correlation: Increased BMI with Smoking
    SNP-Disease Coefficient =
```

Cardiovascular & Hematology

```
Polymorphism in FGB
  Locus 4q28
  SNP Identifier: rs1800790
  Genotype: GA
  Clinical Correlation: Increased Plasma Fibrinogen Levels
    SNP-Disease Coefficient =
  Population: Caucasian
Polymorphism in MTHFR
  Locus 1p36.3
  SNP Identifier: rs1801133
  Genotype: TT
  Clinical Correlations:
    Decreased Longevity
    SNP-Disease Coefficient =
    Hyperhomocysteinemia, Neutralizable with Folate Supplementation
    SNP-Disease Coefficient =
    Increased risk of Premature Cardiovascular Disease
      Odds Ratio = 3.00
      SNP-Disease Coefficient =
    Increased risk of Ischemic Stroke
      Odds Ratio = 1.24
      SNP-Disease Coefficient =
    Increased risk of Neural Tube Defect
      Odds Ratio = 7.20
      SNP-Disease Coefficient =
    Increased risk of Pulmonary Embolism with Oral Contraceptive Pills
    SNP-Disease Coefficient =
    Increased risk of Thrombosis with Smoking
    SNP-Disease Coefficient =
    Increased risk of Preeclampsia
    SNP-Disease Coefficient =
    Mother at Increased Risk of Having a Child with Down Syndrome
      Odds Ratio = 1.91
      SNP-Disease Coefficient =
    Increased risk of Primary Open-angle Glaucoma
      Odds Ratio = 2.38
      SNP-Disease Coefficient =
    Increased risk of Migraine with Aura
      Odds Ratio = 2.05
      SNP-Disease Coefficient =
    Increased risk of Depression
      Odds Ratio = 1.69
      SNP-Disease Coefficient =
    Increased risk of Schizophrenia
      Odds Ratio = 1.48
      SNP-Disease Coefficient =
    Increased Need for B-vitamin Nutritional Supplementation
    SNP-Disease Coefficient =
  Population: Caucasian
Polymorphism in F5
  Locus 1q23
  SNP Identifier: rs6025 (Factor V Leiden Mutation)
  Genotype: AA
  Clinical Correlations:
    Increased risk of Venous Thromboembolism
      Odds Ratio = 18.00, Lifetime Risk = 10%
    SNP-Disease Coefficient =
    Increased risk of Thrombosis if Nonsmoker, Normal Weight, Under 40 y/o
      10-year Absolute Risk = 3%
    SNP-Disease Coefficient =
    Increased risk of Thrombosis if Smoker, Overweight, Over 60 y/o
      10-year Absolute Risk = 51%
    SNP-Disease Coefficient =
    Increased risk of Deep Vein Thrombosis Recurrence after First DVT
      Odds Ratio = 2.94
    SNP-Disease Coefficient =
    Increased risk of Late Fetal Loss when Pregnant
      Odds Ratio = 3.00
    SNP-Disease Coefficient =
    Increased risk of Thromboembolism with Pregnancy
      Odds Ratio = 9.30
    SNP-Disease Coefficient =
    Significantly Increased risk of Thromboembolisms if on Oral Contraceptive Pills
    SNP-Disease Coefficient =
  Population: Caucasian
Polymorphism in F7
  Locus 13q34
  SNP Identifier: rs6046
  Genotype: TT
  Clinical Correlations:
    Factor VII Deficiency
    Protection against Myocardial Infarction
      Odds Ratio = 0.47
    SNP-Disease Coefficient =
    Increased risk of Recurrent Venous Thrombosis
      Odds Ratio = 1.30
    SNP-Disease Coefficient =
  Population: Caucasian
          Polymorphisms detected in FGB, MTHFR, F5, and F7
          Loci: 4q28 & 1p36.3 & 1q23 & 13q34
          Haplotype Identifier: rs1800790 & rs1801133 & rs6025 & rs6046
          Haplotype: A-T-A-T
          Clinical Correlation: Increased risk of Recurrent Venous Thrombosis
          Odds Ratio = 5.10
    SNP-Disease Coefficient =
          Population: Caucasian
```

Pharmacology & Cardiovascular

```
Polymorphism in VKORC1
  Locus 16p11.2
  SNP Identifier: rs9923231
  Genotype: AA
  Clinical Correlation: Increased Sensitivity to Warfarin (Coumadin)
    SNP-Disease Coefficient =
  Population: All
Polymorphism in KIF6
  Locus 6p21.2
  SNP Identifier: rs20455
  Genotype: TC
  Clinical Correlations:
    Increased risk of Coronary Artery Disease
      Odds Ratio = 1.24 (Caucasian Female)
      Odds Ratio = 1.50 (Caucasian Male)
    SNP-Disease Coefficient =
    High-dose Atorvastatin Therapy (80 mg) Reduced Risk of Death or Major Cardiovascular Events
      by 41% Compared with Standard-dose Pravastatin Therapy
      Hazard Ratio = 0.59
    SNP-Disease Coefficient =
Polymorphism in KCNE2
  Locus 21q22.1
  SNP Identifier: eg2525
  Genotype: GC
  Clinical Correlations:
    Drug (Clarithromycin) Induced Long QT Interval and Cardiac Ventricular Arrhythmias
    SNP-Disease Coefficient =
  Population: Caucasian
WARNING: Potential fatal interaction with Clarithromycin. Avoid clarithromycin. Educate individual
about this potential fatal interaction. Make sure medical records clearly highlight this potential fatal
interaction.
```

Cardiovascular

```
Polymorphism in AGT
  Locus 1q42-q43
  SNP Identifier: rs699
  Genotype: CC
  Clinical Correlations:
    Increased risk of Salt-Sensitive Hypertension
    SNP-Disease Coefficient =
    Increased risk of Pregnancy-Induced Hypertension
    SNP-Disease Coefficient =
  Population: Caucasian
“Using individualized dose adaptation, a significant reduction of bleeding complications can be
expected, especially in the initial drug saturation phase. Furthermore, concomitant application of low
dose vitamin K may significantly reduce intra-individual coumarin dose variation and, thus, may
stabilize oral anticoagulation therapy. The use of new pharmacogenetics-based dosing schemes and the
concomitant application of low-dose vitamin K with coumarins will decidedly influence the current
practice of oral anticoagulation and greatly improve coumarin drug safety.” (Oldenburg, J. Thromb
Haemost. 2007 Sep; 98(3): 570-8.
```

Rheumatology

```
Polymorphism in GDF5
  Locus 20q11.2
  SNP Identifier: rs143383
  Genotype: CT
  Clinical Correlations:
    Osteoarthritis of the Hip & Knee
      Odds Ratio = 1.28 (Caucasian)
      Odds Ratio = 1.79 (Chinese & Japanese)
    SNP-Disease Coefficient =
    Decreased Height (Standing Height)
    SNP-Disease Coefficient =
```

Endocrinology & Traits

```
Polymorphism in FTO
  Locus 16q12.2
  SNP Identifier: rs9939609
  Genotype: AA
  Clinical Correlations:
    Increased risk of being overweight during childhood
      Odds Ratio = 1.27
    SNP-Disease Coefficient =
    Increased risk of obesity during childhood
      Odds Ratio = 1.35
    SNP-Disease Coefficient =
    Increased risk of being overweight during adulthood
      Odds Ratio = 1.38
    SNP-Disease Coefficient =
    Increased risk of obesity during Adulthood
      Odds Ratio = 1.67
    SNP-Disease Coefficient =
    Significant Increased in Weight Over 25-years Starting in Youth
    SNP-Disease Coefficient =
    Diabetes Mellitus, Type II due to Increased Obesity from this Gene
      Odds Ration = 1.55
    SNP-Disease Coefficient =
  Population: Caucasian
  Polymorphism Level = −1
Polymorphism in TUB
  Locus 11p15.5
  SNP Identifier: rs2272382
  Genotype: AA
  Clinical Correlations:
    Increased risk of obesity during Adulthood
      Odds Ratio = 1.32
    SNP-Disease Coefficient =
    Diet found to Contain Increased Glycemic Load
    SNP-Disease Coefficient =
    Derive Less Energy from Fat in Diet
    SNP-Disease Coefficient =
  Population: Caucasian
```

Hematology

```
Polymorphism in ABO
  Locus 9q34
  DIP Identifier: eg22696
  Genotype: deletion/deletion
  Clinical Correlation: Blood Type = O
    SNP-Disease Coefficient =
  Population: Caucasian
  Polymorphism Level = 0
```

Pharmacology

```
Polymorphism in CACNA1S
  Locus 1q32
  SNP Identifier: eg36558
  Genotype: GA
  Clinical Correlation: Malignant Hyperthermia
    SNP-Disease Coefficient =
WARNING: Extreme caution with general anesthesia. Educate individual about that they may be
genetically predisposed to malignant hyperthermia. Reiterate the importance of informing all
physicians, especially anesthesiologists and surgeons, about this predisposition. Make sure this
predisposition appears clearly in their medical record.
```

Traits & Psychiatry

```
Polymorphism in GABBR2
  Locus:
  SNP Identifier:
  Genotype:
  Clinical Correlations: Increased risk of Nicotine Addiction
    SNP-Disease Coefficient =
  Population: Caucasian
Polymorphism in CYP2B6
  Locus: 19q13.2
  Haplotype Identifier: rs3745274 & rs2279343
  Haplotype: TT-GG
  Clinical Correlations:
    Indicator of Buproprion Success for Treatment of Nicotine Addiction (Abstinence at 10 Weeks &
      Six Months)
      Odds Ratio = 2.97
    SNP-Disease Coefficient =
  VReduced Dosage required with Efavirenz
    SNP-Disease Coefficient =
  Population: Caucasian
```

Ear, Nose and Throat & Pharmacology

```
Polymorphism in MTRNR1
  Locus: Mitochondrial DNA
  SNP Identifier: eg1555
  Genotype: AG
  Clinical Correlations:
    Deafness caused by exposure to Aminoglycosides (a class of antibiotics)
    SNP-Disease Coefficient =
    Late-onset Sensorineural Deafness
    SNP-Disease Coefficient =
  Population: All
WARNING: Avoid all aminoglycosides. Refer to ENT specialist due to the possibility of developing
late-onset sensorineural deafness. Educate individual about importance of avoiding aminoglycosides for
their entire life and about the possibility about late-onset deafness. Recommend for individual's
extended family members to be tested for the mutation along the maternal lineage so that they, too, will
know whether they need to strictly avoid all aminoglycosides.
Discuss with individual the symptoms of restrictive cardiomyopathy and that if any symptoms manifest,
they should seek medical attention. Physicians should have low threshold for pursuing cardiomyopathy
work-up if individual presents with any symptoms. Start therapy sooner rather than later.
```

Gastroenterology & Pharmacology

```
Polymorphism in TPMT
  Locus 6p22.3
  SNP Identifier: eg55417
  Genotype: CA
  Clinical Correlation: Possible susceptibility to 6-mercaptopurine Toxicity
    SNP-Disease Coefficient =
WARNING: 6-MP (Mercaptopurine) and Azathioprine Toxicity. This is a very important detection for
this individual because they are predisposed to Crohn Disease, and Azathioprine or 6-MP are often used
as part of the medical treatment. Individual should be educated about this sensitivity and it should
clearly annotated on their medical records. Individual should be instructed to discuss this sensitivity
with their gastroenterologist, as it will be important if they are ever diagnosed with Crohn Disease.
```

Metabolic and Rare Diseases & Ear, Nose, and Throat

```
Polymorphism in SLC26A4
  Locus 7q31
  SNP Identifier: eg25662
  Genotype: AC
  Clinical Correlation: Carrier of Pendred Syndrome
    SNP-Disease Coefficient =
  Population: All
Pendred syndrome is the most common form of deafness and is associated with developmental
abnormalities of the cochlea, sensorineural hearing loss, and diffuse thyroid enlargement (goiter). This
individual is a carrier of the most common mutation causing Pendred syndrome. They should be
educated that, because it is a recessive disease, they will not be affected by this mutation. However, this
will be important to discuss further when they are thinking about having children. At that time, the other
parent of their child should also have their SLC26A4 gene checked for mutations so as to properly
ascertain risk of their child having Pendred syndrome.
```

Metabolic and Rare Diseases

```
Polymorphism in HEXA
  Locus 15q23-q24
  DIP Identifier: eg27487
  Genotype: Insertion/Deletion
  Clinical Correlation: Carrier of Tay Sachs Disease
    SNP-Disease Coefficient =
  Population: All
Tay-Sachs disease is an autosomal recessive progressive neurodegenerative disorder which, in the
classic infantile form, is usually fatal by age 2 or 3 years old. This individual is a carrier of the most
common mutation causing Tay Sachs. They should be educated that, because it is a recessive disease,
they will not be affected by this mutation. However, this will be important to discuss further when they
are thinking about having children. At that time, the other parent of their child should also have their
HEXA gene checked for mutations so as to properly ascertain risk of their child having Tay Sachs.
Polymorphism in MUT
  Locus 6p12.3
  SNP Identifier: eg33094
  Genotype: AT
  Clinical Correlation: Carrier of Methylmalonic Aciduria
    SNP-Disease Coefficient =
  Population: All
Methylmalonic aciduria is an autosomal recessive metabolic disorder that has a wide clinical spectrum,
ranging from a benign condition to fatal neonatal disease. This individual is a carrier of the most
common mutation causing Methylmalonic Aciduria. They should be educated that, because it is a
recessive disease, they will not be affected by this mutation. However, this will be important to discuss
further when they are thinking about having children. At that time, the other parent of their child should
also have their MUT gene checked for mutations so as to properly ascertain risk of their child having
Methylmalonic Aciduria.
Polymorphism in COL7A1
  Locus 3p21.3
  SNP Identifier: eg7491
  Genotype: CA
  Clinical Correlation: Carrier of Epidermolysis Bullosa Dystrophica
    SNP-Disease Coefficient =
  Population: All
Epidermolysis Bullosa Dystrophica, which may be autosomal recessive or autosomal dominant, is a
dermatologic disorder that causes severe blistering and scarring, sometimes with resulting disfigurement
and significantly increased risk of infection. Due to involvement of the esophagus, malnutrition can
occur. Individuals are also at an increased risk for skin cancer. The majority of individuals with this
disease die before the age of 30.
```

Traits

```
Polymorphism in 40 SNPs (used as Universal Identifier)
  Loci: 40 distinct loci throughout genome
  SNP Identifiers: x40
  Genotype: (40 bp Genotype)
  Clinical Correlations: Universal Identifier (no disease-associations for any SNP)
    SNP-Disease Coefficient =
  Population: All
This represents a ‘genetic fingerprint’ of the individual and no other individual on the planet will have the
same universal identifier. In all populations, the probability of discrimination is greater than
0.999999999999. This is useful as a way to identify the individual, or as life-long security method to
always be able to identify a newborn, child, head of state, person in the military, person under
investigation or watch, or high profile individual, such as an executive of a large corporation.
Polymorphism in (ETHNICITY)
  Locus: 2q21
  SNP Identifier:
  Genotype:
  Clinical Correlations:
  Population:
    SNP-Disease Coefficient =
*SNP-Disease Coefficient: 0 = Two or more contradictory studies; 0.25 = Single study with single study. population containing under 250 individuals; 0.50 = Single study with single study population containing over 250 individuals; 0.75 = Single study with two or more study populations with each containing under 250 individuals; 1 = Monogenic disorder; polymorphism found to segregate with disease or found within gene that has previously been associated with disease or likely to be associated with disease; 1 = Single study with two or more study populations (same or different ethnicities), each containing 250-999 individuals and each giving similar results; 1.25 = Single study with two or more study populations (same or different ethnicities), each containing over 1,000 individuals and each giving similar results; 1.50 = One primary study and one replication study, each with similar findings (same disease association and same direction of risk); 1.75 = One primary study with two or more replication studies, each with similar findings (same disease association and same direction of risk); 2 = Two or more GWAS with similar results.
```

A report for the individual can include anonymous individual information with relevant factors (FIG. 4A) and a score report for organ system(s), overall genetic health score (for example, with information derived as shown in FIGS. 9, 10). The report for the individual may include the following:

Individual Summary: While no polymorphisms were detected in the primary high-risk but low frequency genetic variants associated with breast cancer (in the BRCA1 and BRCA2 genes), five breast cancer associated genetic variants were detected throughout your genome. This increases your risk of breast cancer significantly. (Odds ratios are be added or multiplied together for a total odds ratio, or converted to relative risks depending on the disease prevalence or incidence statistics, as previously discussed) with a genetic predisposition towards estrogen-receptor positive breast cancer.) Without any risk factors, your lifetime risk of breast cancer is approximately 7-13%. With these genetic risk factors, your personalized lifetime risk of breast cancer is increased, and may be as high as approximately 24%. The majority of the risk for you exists after the age of 40.

### Recommendations or Preventive Measures:

Follow-up: Due to your significantly increased risk of breast cancer based on the genetic analysis conducted, it is recommended that you follow-up with a women's health breast cancer specialist that may be able to discuss potential preventive measures. Due to your increased risk, it may be important for you to visit with this specialist at least once a year in order to monitor for signs of the disease.

Screening: Research has shown that increased radiation to the chest may further increase the risk of breast cancer in individuals who have genetic variants predisposing them to breast cancer. Because of this, you may try to avoid chest x-rays, mammograms, ct-scans, and any other type of radiation whenever possible, but always consult with your physician. Instead, radiologic screening with non-radiologic devices, such as MRI's or Ultrasounds may be warranted.

Lifestyle Modifications: A) Smoking cigarettes has been shown to increase the risk of many different types of cancer, including breast cancer, so it is recommended that you discuss this with your physician and consider quitting smoking. B) The medication Buproprion (Zyban) has been shown to increase some people's ability to quit smoking. Based on your specific genetic profile, you are almost 200 times more likely to quit smoking if you use Burproprion. You should discuss this medication along with a plan to quit smoking with your primary care physician. C) Being overweight has also been shown to increase the risk of breast cancer so it is recommended that you may also want to consider losing weight, such as between 10 to 20 pounds. D) You do have one or more genetic variants that preliminary evidence suggests may cause you to gain weight if you smoke. Therefore, quitting smoking may significantly help with weight loss in the long-run.

Medications: Because you are predominantly predisposed to estrogen-receptor breast cancer, it is recommended that you discuss this with your physician and that you may want to discontinue any oral contraceptive pills and, later in life, avoid hormone replacement therapy which may be given if you are experiencing severe symptoms during menopause. A) Genetic variants have been detected that may affect the way you body responds to some medications used to treat breast cancer. (Please discuss this information with your physician before you start or stop any medications.) B) It was found that you may be resistant to Topoisomerase II-Targeting Chemotherapeutic Drugs such as Mitoxantrone and therefore this medication may have limited efficacy for you in treating breast cancer. C) It was found that, based on a specific genetic variant, you may respond very well to the Aromatase Inhibitor Letrozole. D) If chemotherapy for breast cancer is required, based on the current genetic analysis, you are not at an increased risk for chemotherapy-induced leukemia (AML), which is a type of cancer of the blood that some people get as a serious side effect of their initial chemotherapy treatment for cancer.

Score By Organ System (assuming all other SNPs tested were not associated with disease): Cardiovascular Score: Green; Dermatology Score: Green; Eyes, Ears, Nose, Throat Score: Green; Obstetrics: Green; Oncology: Red; Pharmacology: Yellow; Women's Health Score: Red; Urology: Green.

### Overall Genetic Health Score: Yellow

Clinician Summary: Based on genetic testing and analysis conducted and based on current literature, individual is at significantly increased risk of breast cancer, possible having greater than a 20% lifetime risk of breast cancer if the odds ratios for all of her breast cancer-associated genetic variants are converted to relative risks, analyzed and multiplied together (they may also be added together, with the decision based on methodology used to determine multifactorial risk. For further information about algorithm used in this analysis, please contact us or read the section of the summary that discusses the algorithm methodology). The individual has a predisposition towards estrogen receptor-positive breast cancer. A) Genetic profile indicates Buproprion may significantly help this individual to quit smoking. B) If chemotherapy for breast cancer becomes necessary: i) Aromatase Inhibitors may be more effective. ii) Topoisomerase II-Targeting Chemotherapeutic may be less effective. iii) Individual is not be at an increased risk of chemotherapy-induced AML.

### Example 7

### Prophetic

### Organ System Score and Overall Genetic Health Score with More Subject Information

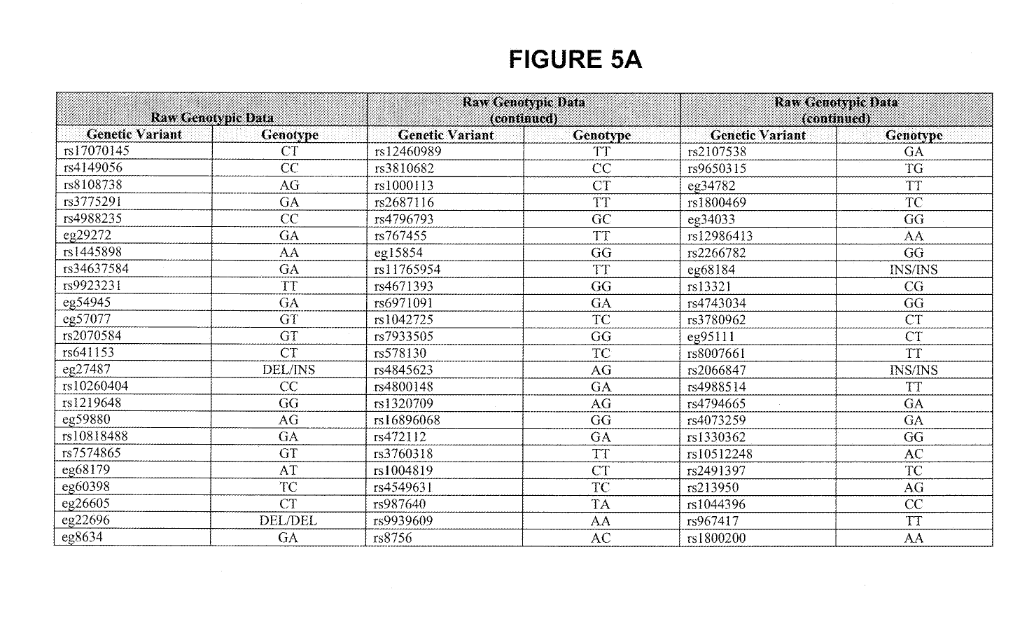

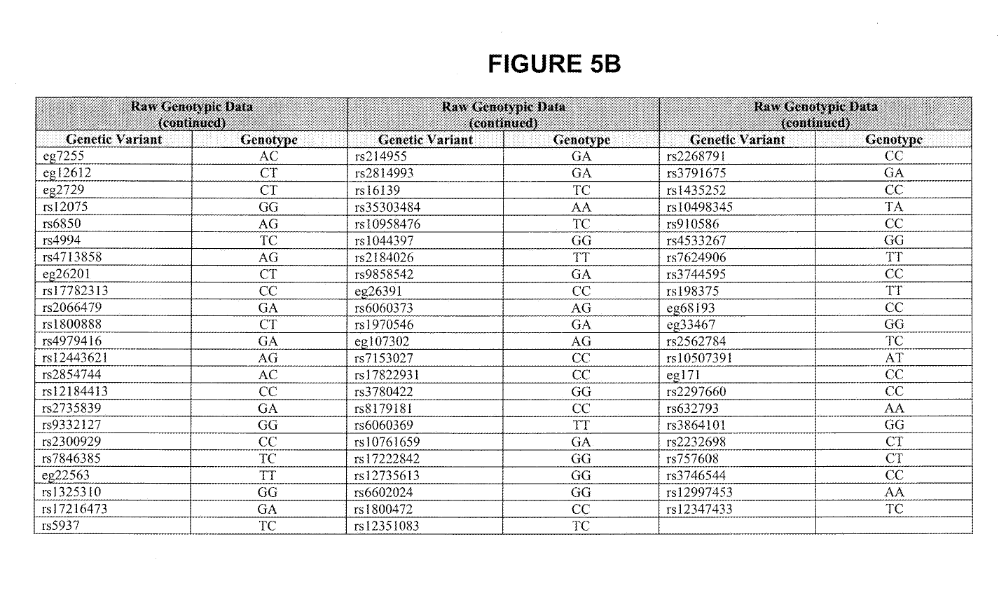

An individual with information as shown in FIG. 4B chooses the Full Genome Analysis Panel to determine her risk or predisposition to a number of phenotypes, such as traits. Raw genotypic data is generated, such as shown in FIG. 5, from an internal or outside source, such as genetic testing conducted at a CLIA-certified laboratory. This data is then entered into the information technology system and preliminary analysis is conducted that associates the specific genetic variants and their specific genotypes with phenotypes, by cross-referencing a database, such as the Predictive Medicine Database, and may then also calculate risk or carrier status again by cross-referencing a database, such as the Predictive Medicine Database. As can be seen in FIG. 6A-D, the data can be viewed many different ways, such as without any filters (Results View=All, FIG. 6A), with the results filtered by GVP score (Results View=GVP Score ≧1.5, FIG. 6B), or the results filtered by only monogenic diseases (Results View Monogenic, FIG. 6C), or the results can also be filtered by more than one field, such as results filtered by specific replication status or those that are monogenic (Results View=Replicated or Monogenic, FIG. 6D). This allows for complete operator control over viewing the preliminary associations detected, and allows for the preliminary results to be filtered by any field, either manually or automatically (for example, if preset when the panel was ordered). The Results View=Phenotypes, FIG. 6E-G, allows for the viewing of all the phenotypes detected along with the various phenotype rating scales and their associated organ systems and medical specialties. Phenotypes can further be filtered, if necessary, based on any of these fields. After the data is filtered, it is then fully analyzed and a genetic report is produced.

### Example 8

### Prophetic

### Preventive Measures (PMs) Based on Preventive Medicine Recommendations or Interventions (PMRI's) for Alzheimer's Disease

An individual's genotypic profile or phenotypic profile or both indicates that he or she is at an increased risk for Alzheimer's Disease and specifically contain the APOE-E4 genetic variant, then preventive measures (PMs), based on clinical and scientific research concerning preventive medicine recommendations and interventions (PMRI's) related to the prevention, and delaying the onset, of Alzheimer's Disease may be included in the Genetic Report. PMs based on PMRI's appear in FIG. 12 under the “Prevention” heading. For example, individuals carrying the APOE-E4 genetic variant are at significantly increased risk of Alzheimer's Disease if they experience a traumatic brain injury. A PM based on a PMRI specific for APOE-E4 carriers states this association and recommends the risk of head injury may want to be avoided, such as by avoiding contact sports and by wearing a protective helmet while bike riding, skate boarding, roller blading, or any other sport where head trauma is a possibility. (Plassman et al. Neurology 55(8): 1158-1166 (2000); Koponen et al. Neurology 63(4): 749-750 (2004))

Two Genetic Reports are generated, one for the individual and the other for a physician, which contains PMs based on PMRI's that are specifically tailored for each. For example, the PM based on the PMRI for the patient may include a statement similar to “modern brain scans now enable physicians to non-invasively detect the early signs of Alzheimer's disease in a person's brain and to also follow the progression of the disease” while the PM based on the PMRI for the physician may include a statement similar to “FDDNP-PET brain scans are available from UCLA Medical Center in Los Angeles, Calif. and now provide for a way to detect the development and progression of plaques and tangles in the brain”.

The PMs based on the PMRIs for both Genetic Reports further may include one or more of the following types of information:

A) Disease Education: Description of Alzheimer's disease, such as its cause, pathology or presentation.

B) Disease Warning Signs and Symptomatology:

A description of symptoms of Alzheimer's disease, such as it being a degenerative and terminal disease that affects a person's memory, cognition, and mood.

Symptomatology may be referenced based on a scale, such as time. For example, memory loss may be an initial symptom early on in the course of the disease while increased impairment in learning, and possibly language, occurs at a later stage.

Examples of symptoms that may indicate that the individual should consult with their healthcare provider include cognitive issues that may first start to manifest during daily activities, such as repeatedly misplacing car-keys or a decreasing ability to balance a checkbook.

C) Lifestyle Modifications

Increasing physical exercise (such as walking the golf course instead of using the golf cart, or taking walks after lunch or dinner) (Larson et al. Ann Intern Med 144(2): 73-81 (2006))

Increasing mental exercise (such as playing chess, learning a new language, or doing a daily crossword puzzle) (Willis et al. JAMA 296(23): 2805-2814 (2006))

Protecting your head from any type of trauma (such as if a child is considering whether or not to participate in a contact sport such as ice hockey) (Plassman et al. Neurology 55(8): 1158-1166 (2000); Koponen et al. Neurology 63(4): 749-750 (2004))

Drinking coffee, even up to three or more cups per day. Reflex testing shows that caffeine consumption during the day should not have a negative affect upon your quality of sleep at night. Eskelinen, M. H., T. Ngandu, et al. (2009).

“Midlife Coffee and Tea Drinking and the Risk of Late-Life Dementia: A Population-Based CAIDE Study.” Journal of Alzheimer's Disease 16(1): 85-91.

Diets low in animal fat, such as the Mediterranean-style diet, have been shown to decrease risk of Alzheimer's disease (Soft et al. BMJ 337(sep11_{—}2): a1344-(2008))

D) Prescription Medications

Statins (such as atorvastatin, brand name Lipitor®) have been shown to decrease the risk of Alzheimer's disease (Jick et al. The Lancet 356(9242): 1627-1631 (2000))

E) Over-the-counter Medications

Taking non-steroidal anti-inflammatory medications (such as Ibuprofen) have been shown to decrease the risk of Alzheimer's disease (Vlad, et al. Neurology 70(19): 1672-1677 (2008))

F) Monitoring Modalities

Brain Scans (such as PIB-PET and FDDNP-) provide indications of disease onset and progression (Shin et al. “Multitracer PET imaging of amyloid plaques and neurofibrillary tangles in Alzheimer's disease.” NeuroImage, In Press, Corrected Proof)

Mini-mental exams provided by a healthcare provider can also assess disease onset and progression (Commenges et al. Epidemiology 3(2): 185-188 (1992))

G) Vitamins. Herbs, or Alternative Treatments

Omega-3 Fatty Acids, such as those found in fish and also Fish Oil supplements, have been found to decrease the risk of Alzheimer's disease (Morris et al. Arch Neurol 60(7): 940-946 (2003))

Yoga or meditation to help decrease stress may decrease the risk of Alzheimer's disease (Kidd. Altern Med Rev. 13(2):85-115 (2008))

H) Associated Diseases or Conditions

High blood pressure—Lowering blood pressure to the normal range has been shown to reduce the risk of Alzheimer's disease (Forette et al. Arch Intent Med 162(18): 2046-2052 (2002))

I) Current Treatments

Medications called “Acetylcholinesterase inhibitors” have been shown to slow the progression of Alzheimer's disease and may help with some symptoms. (Rogers et al. Neurology 50(1): 136-14 (1998).). Examples include Donepezil (brand name Aricept®), Rivastigmine (brand name Exelon®), and Galantamine (brand name Reminyl®).

Medications called “NMDA antagonists” have also been shown to slow progression of Alzheimer's disease and may help with some symptoms. (Emre et al. Journal of Alzheimer's Disease 14(2): 193-199 (2008)). Examples include Memantine (Namenda®)

J) Future Treatments

A new medication called “PBT2” is currently in clinical testing and data indicates that it may help treat Alzheimer's disease. (Lannfelt et al. The Lancet Neurology 7(9): 779-786 (2008))

K) Being Connected to a Medical Professional

Recommendation or referral to see a Neurologist or Preventive Medicine Specialist who may then be able to assess a base-line status and follow the patient as they oversee the preventive medicine recommendations as well as continuing to assess disease development or progression or both.

Recommendation or referral to see a Nutritionist in order to assess the patient's diet in light of their increased risk for Alzheimer's disease

L) Common Misconceptions

Vitamin C supplementation has been shown in numerous studies to not be significantly beneficial in the prevention of Alzheimer's disease. (Gray, M. L. A. P. K. C. J. C. S. B. W. M. J. D. B. L. T. E. L. Journal of the American Geriatrics Society 56(2): 291-295 (2008); Luchsinger et al. Arch Neurol 60(2): 203-208 (2003))

### Example 9

### Database Construction

An example of entries for a deterministic (monogenic/mendelian) phenotype, such as a condition, (to determine carrier status), and a predisposition (polygenic or multifactorial) phenotype, such as a condition (to determine risk), into a database, such as a Predictive Medicine Database, with the various fields filled in, is:

Deterministic (Monogenic/Mendelian) Condition

Disease=Epidermolysis Bullosa Simplex

Journal Article=Chan, Y. M., Q. C. Yu, et al. (1993). “The genetic basis of Weber-Cockayne epidermolysis bullosa simplex.” Proceedings of the National Academy of Sciences of the United States of America 90(15): 7414-7418.

Gene Name (from NCBI databases)=Keratin 5

Gene Symbol(s) (from NCBI databases)=KRT5, K5, CK-5, CK5, DDD, EBS2, KRT5A

Gene Locus (from NCBI databases)=12q12-q13

Exact Genetic Variant Identification (from article, NCBI or Ensemble databases)=1161S

50 bp downstream of variant (from Ensemble database)=

TGTCAACCAGAGTCTCCTGACTCCCCTCAACCTGCAAATCGACCCCAGCA

50 bp upstream of variant (from Ensemble database)=

CCAGAGGGTGAGGACCGAGGAGCGCGAGCAGATCAAGACCCTCAACAATA

IUPAC Nucleotide Code=K

Exact Position on Chromosome 12 (from Ensemble database, coordinate system)=51199866

Strand Direction (from article, NCBI or Ensemble databases)=Reverse

Location in Gene (from article, NCBI or Ensemble databases)=Exon

Amino Acid Position (from article, NCBI or Ensemble databases)=161

Amino Acid Change (from article, NCBI or Ensemble databases)=Ile→Ser

Function: Missense

Allele I=T

Allele 2=G

Phenotype-Associated Allele=G

Genetic Risk Prediction Algorithm Assessment, also referred to as Prediction of Effect of Genetic Variant Algorithm Value—FANS: Risk Level=High; Risk Type=Mis-sense (Non-Conservative Change)

Inheritance=Autosomal Dominant (AD)

Replication Status=Monogenic/Replicated

GVP Rank=Mono (Monogenic genetic variants are assigned a GVP Rank=Mono)

Genetic Variant-Disease Coefficient (GVDC, also referred to as GVP Score)=2

Genetic Variant-Phenotype Clinical Significance Rating (CSR, also referred to as GVP Triage)=2

Study Type=Case Study

Journal Article Author's Name=Chan et. al.

Date of Publication=1993

Name of Journal=Proceedings of the National Academy of Sciences of the United States of America

Primary Reference=Chan, Y. M., Q. C. Yu, et al. (1993). “The genetic basis of Weber-Cockayne epidermolysis bullosa simplex.” Proceedings of the National Academy of Sciences of the United States of America 90(15): 7414-7418.

Other Reference(s)=Ehrlich, P., V. P. Sybert, et al. (1995). “A Common Keratin 5 Gene Mutation in Epidermolysis Bullosa Simplex-Weber-Cockayne.” J Investig Dermatol 104(5): 877-879.

Predisposition or Risk (Polygenic or Multifactorial) Condition

Disease=Inflammatory Bowel Disease

Journal Article=Labbe, C., P. Goyette, et al. (2008). “MAST3: a novel IBD risk factor that modulates TLR4 signaling.” Genes Immun. 9(7): 602-12.

Gene Name (from NCBI databases)=Microtubule associated serine/threonine kinase 3

Gene Symbol(s) (from NCBI databases)=MAST3, KIAA0561

Gene Locus (from NCBI databases)=19p13.11

Exact Genetic Variant Identification (from article or NCBI databases or both)=rs8108738

Location in Gene=Exon

Amino Acid Position=861

Amino Acid Change=Ser→Gly

Strand Direction (from article or NCBI databases)=Forward

Function=Missense

Allele I=A

Allele 2=G

Phenotype-associated Allele=G

Genetic Risk Prediction Algorithm Assessment, also referred to as Prediction of Effect of Genetic Variant Algorithm Value—PupaSuite: NON_SYNONYMOUS_CODING; ESE: sc35, ENST00000262811, [score: 3.67 (G), new score: 3.22 (A)—Maintain (−0.45)]; ESE: sf2, ENST00000262811, [score: 4.17 (G), new score: 4.62 (A)—Maintain (0.45)]; ESE: sf2, ENST00000262811, [score: 2.23 (G), new score: 1.30 (A)—Lose (−0.93)]; MutDB: Not found within database; FastSNP: Risk level=Low-Medium (2-3); Missense (conservative); Splicing regulation; SNPs3D: SVM Profile=1.13; VisualSNP: Risk Level=Medium; Mis-sense (Splicing Regulation)

Risk Value=1.19

Risk Type=OR

Risk Value Confidence Interval=1.05-1.34

Risk Value p-value=0.002

Cumulative or Absolute Risk or Other Value=Not stated

MAF=0.468

Specific Population(s)=Italian, Canadian, United States

Incidence of non-phenotype associated allele in disease cohort=Data not supplied

Incidence of phenotype-associated allele in control cohort=Data not supplied

Total number in disease cohort=1105

Inheritance=Not stated

Replication Status=Not Replicated

Genetic Variant-Disease Coefficient (also referred to as GVP Score)=1

Genetic Variant-Phenotype Clinical Significant Rating (CSR, also referred to as GVP Triage)=2

SNP Rank=1

Study Type=Combined Association Mapping Study & Case-Control Study

Journal Article Author's Name=Labbe et al.

Date of Publication=2008

Name of Journal=Genes and Immunity

Primary Reference=Labbe, C., P. Goyette, et al. (2008). “MAST3: a novel IBD risk factor that modulates TLR4 signaling.” Genes Immun. 9(7): 602-12.

Other References=None

### Example 10

### Prophetic

### Use of the Methods of the Present Invention to Improve, Rescue and/or Resurrect Clinical Trials

A medication in phase II human clinical trials is associated with an adverse drug event in 10% of the study participants, such as neutropenia. Since the exact cause of the neutropenia can not be ascertained, the pharmaceutical company conducting the trial is be unable to determine who is at risk for neutropenia and who is not. Utilizing the Research & Clinical Trial Panel this additional genetic data is able to be correlated (by regression analysis or other bioinformatic or statistical analysis) with a specific pattern of one or more genetic variants. The Research & Clinical Trial Panel includes hundreds of genes that contain genetic variants of clinical significance and the panel acts as a screen, so that instead of knowing exactly what gene or genetic variant to test for, instead many different genes are tested for and/or analyzed all at once, thereby allowing the results to identify any genetic variants that are correlated with the phenotype of interest (such as an adverse drug event) and therefore the results do not require a preconceived notion regarding what genetic variant or what correlation to test.

It is found that a statistically significant number of participants who have the adverse drug event carry a genetic variant in their CFTR gene on chromosome 7. This genetic variant is associated with cystic fibrosis or congenital bilateral absence of the vas deferens when it exists in trans with another CFTR disease-associated genetic variant. However genetic testing and/or analysis shows that when exposed to the specific medication being tested in this clinical trial, individuals who ‘carry’ this specific genetic variant, meaning they may not have any observable clinical phenotype because they just carry the genetic variant and are not affected by a phenotype, are actually the individuals who have a much higher incidence of neutropenia, possible because of slight molecular changes to the chloride channels in their cells, due to this CFTR genetic variant. Therefore, there now exists a way to differentiate who will have this severe adverse event to the medication and who will not (by conducting genetic testing and/or analysis for this specific genetic variant) and this augments the chances of eventual FDA and/or EU approval, thereby ‘rescuing’ the clinical trial from possible failure.

### Example 11

### Prophetic

### Alternative Use of the Methods of the Present Invention to Improve, Rescue and/or Resurrect Clinical Trials

As another example, a medication under development by a biotech-pharma company is found to have no response in a segment of the population being studied in their research. It is found that 40% of individuals show no response, while the other 60% show a very favorable response to the medication in the treatment of their disease. The Research & Clinical Trial Panel is run on all study participants and the genetic testing and analysis, including bioinformatic and regression analysis, shows that the non-responders have a common genetic variation's genotype in their CYP2D6 gene on chromosome 22 along with two other genetic variations, a genetic variant's genotype in their TPMT gene on chromosome 6 and a genetic variant's genotype in their UGT1A1 gene on chromosome 2. All three of these genetic variant's and their genotypes for a specific genetic profile are able to discern between responders and non-responders. Further research confirms that this specific genetic profile's association with the non-responder phenotype is statistically significant and can be utilized to segment the population according to responder and non-responder based on this medication under investigation. Further research shows that the non-responders actually has a beneficial response if the starting dose of the medication is tripled and now this same genetic profile can be utilized to determine appropriate starting dose in order to augment chances of appropriate response.

### Example 12

### Prophetic

### Use of the Methods of the Present Invention to Evaluate Individuals for Phenotypes Relevant to Living in Close Quarters

Infectious diseases are highly communicable and spread extremely fast when individuals live in close quarters, such as military barracks, dormitories, assisted living centers, skilled nursing facility, or retirement home or community. Psychiatric illness and other phenotypes can also have profound effect and cause severe disruption or even increased morbidity or mortality for the subject or for other occupants in these living situations. The Close Living Quarters Panel allows for genetic testing and/or genetic analysis of the phenotypes that may want to be taken into consideration by either an occupant of one of these places, or by a health care professional or a housing administrator or housing official. The methods of the present invention are used to test and analyze individuals living in a military barracks for genetic variants related to the phenotypes listed in the Close Living Quarters Panel. The results identify individuals who are at much greater risk of meningitis, as well as death from meningitis, and therefore these individuals are singled out and given prophylactic therapy whenever there is a case or suspected case of meningitis at or near where these individuals live.

### Example 13

### Prophetic

### Use of the Methods of the Present Invention for Analysis of Biological Samples

Pathologists, medical examiners, researchers, corporations, police, military, and other entities store and utilize a large number of biological samples from animals and humans so that those biological samples can be studied then or at some time in the future. The Pathology & Tissue Repository Panel is run on either the individual who submits the biological sample or on the biological sample itself. This panel allows for the rapid identification of the biological sample as well as creating a genetic profile of the sample at clinically-relevant genetic variants throughout the genome. This profile (or one or more genetic variant's alleles or genotypes) is stored electronically or on paper, such as in a database, and this database is then be searched to identify tissue (biological samples) that has a specific genetic profile (one or more specific genetic variant's allele or genotypes) so that those tissue samples can then be accessed and utilized. The panel looks at a large number of clinically relevant genetic variants, including all pharmacogenomic-related genetic variants, cancer-related genetic variants, and heart-disease related genetic variants. The panel also assesses the lineage, ancestry, gender and ethnicity of the biological sample so that if that information is not present, it can be ascertained through the use of this panel.

### Example 14

### Prophetic

### Reflex Testing

Using the methods of genetic testing and analysis described herein, an individual is found to be at increased risk for the initial phenotype of myocardial infarction. Reflex testing is then performed for phenotypes that are useful for evaluating the effectiveness of antiplatelet medications (such as acetylsalicylic acid and/or thienopyridines, such as Clopidogrel); the effectiveness of anti-thrombotic medications; the appropriate dosing of anti-thrombotics (such as warfarin); the effectiveness of lipid-lowering medications (e.g., statins); the risk of adverse events with lipid-lowering medications, antiplatelets, and/or anti-thrombotic medications; the risk of depression and/or suicidality due to stress and/or serious illness; modification of risk of myocardial infarction with the consumption of specific foods, caffeine and/or alcohol; the risk of cognitive decline after coronary artery bypass graft surgery; and the risk (and/or carrier status) of sudden death due to cardiac arrhythmias. Results indicating that the individual is at increased or decreased risk, predisposition, or carrier status of a reflex (second round) phenotype, in-turn, triggers further reflex testing. If an increased risk for depression is found than this reflexes to a genetic test and/or genetic analysis of the individual's risk, predisposition, or carrier status for phenotypes relating to the effectiveness and/or dosing of medications used to treat depression (such as selective serotonin reuptake inhibitors). If an increased risk of cardiac arrhythmias is found for the individual than this reflexes to genetic analysis of and/or testing for the individual's risk, predisposition, or carrier status for phenotypes indicating the effectiveness, choice, and/or dose of anti-arrhythmogenic medication; the risk (and/or carrier status) of drug Induced Torsade de Pointes; and the risk (and/or carrier status) of the individual for drug induced long QT syndrome.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of determining the predisposition or carrier status of an individual for two or more phenotypes related to suitability for military service comprising:
(a) identifying by nucleic acid array or sequencing apparatus a set of genetic variants in an individual, wherein each of said genetic variants is correlated with a suitability for military service phenotype;
(b) using a computer to determine the predisposition or carrier status of said individual for at least two phenotypes, wherein said predisposition or carrier status is based on said set of genetic variants;
(c) providing a report of said predisposition or carrier status to said individual, to a health care provider of said individual, or to a third party; and optionally
(d) combining the predisposition or carrier status of said individual for said at least two phenotypes into a suitability for military service score, wherein said score is reported to said individual, to a health care provider, or to a third party.

2. The method of claim 1, wherein said at least two phenotypes comprise an initial phenotype and a reflex phenotype, wherein said reflex phenotype is a phenotype that is not the initial phenotype, and wherein the reporting of the predisposition or carrier status of said individual for the reflex phenotype depends on the outcome of said determination of predisposition or carrier status of said individual for the initial phenotype.

3. The method of claim 1, wherein said at least two phenotypes are at least two phenotypes listed in one or more of the following figures: Military and Armed Forces Panel Alpha (FIG. 16), or Military and Armed Forces Panel Beta (FIG. 17).

4. The method of claim 1, wherein said at least two phenotypes comprises at least five phenotypes.

5. The method of claim 1, wherein said at least two phenotypes comprise:
(a) at least one phenotype that follows monogenic inheritance; and
(b) at least one phenotype that follows multifactorial or polygenic inheritance.

6. The method of claim 1, wherein said at least two phenotypes comprises at least two of the following phenotypes: universal identifier; blood group; extreme high or low intelligence quotient; post traumatic stress disorder susceptibility; adverse reaction to smallpox vaccination; sensitivity to weapons of mass destruction; extreme high or low visual acuity; or athletic ability, or predisposition to specific sports.

7. The method of claim 1, wherein said at least two phenotypes comprises at least two of the following phenotypes: universal identifier; post traumatic stress disorder susceptibility; specific physical exercise regimen for most efficient physical exercise; thrombophilia or thromboembolic disease.

8. The method of claim 6, wherein said at least two phenotypes further comprise at least one of the following phenotypes: thrombophilia or thromboembolic disease; psychiatric illness; personality traits; effect of stimulants on cognition; stressful life events causing depressive symptoms, diagnosable depression, suicidality or anxiety; infectious disease susceptibility.

9. The method of claim 7, wherein said at least two phenotypes further comprise at least one of the following phenotypes: violent behavior; noise-induced hearing impairment or hearing loss; effect of stimulants on cognition; stressful life events causing depressive symptoms, diagnosable depression, suicidality or anxiety; malaria susceptibility; arrhythmogenic right ventricular cardiomyopathy.

10. The method of claim 2, wherein said reflex phenotype is reported when said individual has an increased predisposition or carrier status for said initial phenotype.

11. The method of claim 2, wherein said reflex phenotype is reported when said individual has a decreased predisposition or carrier status for said initial phenotype.

12. The method of claim 2, wherein said reflex phenotype is not reported if the individual has neither a decreased nor increased predisposition nor carrier status for said initial phenotype.

13. The method of claim 2, wherein said reflex phenotype is reported concurrently with said initial phenotype.

14. The method of claim 2, wherein said reflex phenotype is reported subsequently to said initial phenotype.

15. The method of claim 2, wherein the determination of the predisposition or carrier status of the individual for said reflex phenotype is determined subsequently to the determination of the predisposition or carrier status of the individual for said initial phenotype.

16. The method of claim 2, wherein said reflex phenotype is a disease that is positively correlated with said initial phenotype.

17. The method of claim 2, wherein said initial phenotype is a disease and said reflex phenotype is a symptom of said disease.

18. The method of claim 2, wherein said initial phenotype is a disease or disorder and reflex phenotype is a side effect of, or response to, a treatment for said initial phenotype.

19. The method of claim 2, wherein said initial phenotype is thrombophilia or a thromboembolic disorder, and said reflex phenotype is one or more selected from the group consisting of: warfarin suitability; and suitability of anti-thrombotic medications or NSAIDS.

20. The method of claim 2, wherein said initial phenotype is a psychiatric illness, and said reflex phenotype is one or more selected from the group consisting of: treatment-emergent suicidality during treatment with antidepressents; suitability of medications used to treat depression; response rates to standard treatment for late-life depression; aggressiveness or homicidal behavior with schizophrenia; severity or symptomology of schizophrenia; suitability of mood stabilizers or antipsychotic medications; cognitive performance with bipolar disorder; antipsychotic medication induced parkinsonism; and lithium response in mania or bipolar disorder.

21. The method of claim 2, wherein said initial phenotype is effect of stimulus on cognition, and said reflex phenotype is one or more selected from the group consisting of: stimulant induced adverse reactions, and drug addiction.

22. The method of claim 2, wherein said initial phenotype is stressful life events causing depressive symptoms diagnosable depression or anxiety, and said reflex phenotype is one or more selected from the group consisting of: suitability of medications used to treat depression; treatment-emergent suicidality during treatment with antidepressants; and suitability of medication for treatment for anxiety.

23. The method of claim 2, wherein said initial phenotype is infectious disease susceptibility, and said reflex phenotype is one or more selected from the group consisting of: suitability of medication to treat HIV infection; prognosis, rate of progression, CD4 count or viral load with HIV infection; risk of HIV dementia; suitability of medications used to treat infections; severity or prognosis with HCV infection; suitability of medications used to treat hepatitis C virus infection; severity or prognosis with meningococcal disease; age at onset of prion diseases; hepatitis B virus infection prognosis or rate of hepatitis B virus clearance; vaccine-induced immunity to hepatitis B virus infection; glucose-6-phosphate dehydrogenase deficiency; severity, prognosis, mortality, morbidity or parasite load with malarial infection; suitability of medication used to treat malarial infection or for malarial prophylaxis; response to Lepromin; disease and prognosis following M. leprae infection; severity or prognosis of herpes simplex virus infection; and iron deficiency or iron deficiency anemia during malaria season.

24. The method of claim 2, wherein said initial phenotype is malaria susceptibility, and said reflex phenotype is one or more selected from the group consisting of: glucose-6-phosphate dehydrogenase deficiency, severity, prognosis or parasite load with malarial infection; prognosis, mortality or severity with malarial infection; suitability of medication used to treat malarial infection or for malarial prophylaxis; and iron deficiency or iron deficiency anemia during malaria season.

25. The method of claim 2, wherein said initial phenotype is arrhythmogenic right ventricular cardiomyopathy, and said reflex phenotype is one or more selected from the group consisting of: suitability of antiarrhythmogenic medication; and digoxin absorption, metabolism or toxicity.

26. The method of claim 2, wherein said initial phenotype is height or weight, and said reflex phenotype is one or more selected from the group consisting of: response of stature to human growth hormone; diabetes mellitus type II; amount of effort needed to lose weight; dyslipidemia or lipid levels with increased BMI or obesity; change in body fat or lipid levels with specific diets or with exercise; and exercise tolerance, optimal exercise regimen or athletic training regimen for weight management.

27. The method of claim 2, wherein said initial phenotype is susceptibility to bacteremia, sepsis, severe sepsis, septic shock, or systemic inflammatory response syndrome, and said reflex phenotype is one or more selected from the group consisting of: severity of sepsis, septic shock, severe sepsis or systemic inflammatory response syndrome; source of infection, type of bacteria with bacteremia, sepsis, severe sepsis, septic shock or systemic inflammatory response syndrome.

28. The method of claim 2, wherein said initial phenotype is meningococcal disease susceptibility and said reflex phenotypes is severity of meningococcal disease.

29. The method of claim 2, wherein said initial phenotype is tuberculosis susceptibility and said reflex phenotypes is clinical manifestation of tuberculosis infection.

30. The method of claim 2, wherein said initial phenotype is hypertrophic cardiomyopathy and said reflex phenotypes is heart wall thickness with cardiomyopathy.

31. The method of claim 1, wherein said predisposition or carrier status is determined from at least two genetic variants.

32. The method of claim 31, wherein said at least two genetic variants are correlated with the same phenotype.

33. The method of claim 31, wherein said predisposition or carrier status is determined for adverse reaction to smallpox vaccination and at least one of said genetic variants is selected from the group consisting of, or in linkage disequilibrium with, at least one genetic variant selected from the group consisting of: rs839, rs1801133, and rs9282763.

34. The method of claim 31, wherein said predisposition or carrier status is determined for universal identifier and at least one of said genetic variants is selected from the group consisting of, or in linkage disequilibrium with, at least one genetic variant selected from the group consisting of: rs8176747, rs8176741, rs6444724, rs1336071, rs7520386, ABO Chr. 9: 135122733 delG, rs1019629, rs2073383, rs13218440, rs1478829, rs3780962, rs214955, rs13134862, rs1410059, rs7205345, rs321198, rs338882, rs10488710, rs279844, rs6811238, rs1058083, rs13182883, rs8176749, rs560681, rs10092491, rs740598, rs445251, rs1358856, rs1821380, rs1523537, rs7229946, rs8176720, rs2567608, rs9951171, rs1554472, rs1109037, rs2272998, rs987640, rs12997453, rs2503107, rs447818, rs7704770, rs315791, rs6591147, rs985492, rs8176743, and rs8176746.

35. The method of claim 31, wherein said predisposition or carrier status is determined for blood group and at least one of said genetic variants is selected from the group consisting of, or in linkage disequilibrium with, at least one genetic variant selected from the group consisting of: rs8176741, rs 12075, rs 11276, rs8176720, rs8176743, rs8176747, SLC14A1 Chr. 18: 41573550 Y, ABO Chr. 9: 135121239 S, ABO Chr. 9: 135121469 Y, ABO Chr. 9: 135122733 delG, rs2285644, rs1058396, rs5036, rs8176058, rs28399653, rs1135062, rs3894326, rs28362459, rs28362692, and CD151 Chr. 11: 827536 R.

36. The method of claim 31, wherein said predisposition or carrier status is determined for intelligence and at least one of said genetic variants is selected from the group consisting of, or in linkage disequilibrium with, at least one genetic variant selected from the group consisting of: rs8191992, rs2061174, rs363043, rs353016, rs58646131, rs4680, and rs1130233.

37. The method of claim 31, wherein said predisposition or carrier status is determined for post-traumatic stress disorder susceptibility and at least one of said genetic variants is selected from the group consisting of, or in linkage disequilibrium with, at least one genetic variant selected from the group consisting of: rs9296158, rs6277, rs4606, rs 1360780, and rs9470080.

38. The method of claim 31, wherein said predisposition or carrier status is determined for athletic ability or athletic predisposition and at least one of said genetic variants is selected from the group consisting of, or in linkage disequilibrium with, at least one genetic variant selected from the group consisting of: rs11689011, rs1815739, rs1867785, rs895436, rs4035887, rs4646994, rs17602729, MSTN Chr. 2: 190635190 R, MTCYB Mito: 15615 R, MTCYB Mito: 14846 R, MTCYB Mito: 15497 R, and MTTG Mito: 10010 Y.

39. The method of claim 31, wherein said predisposition or carrier status is determined for thrombophilia or thromboembolic disease and at least one of said genetic variants is selected from the group consisting of, or in linkage disequilibrium with, at least one genetic variant selected from the group consisting of: rs6025, rs6046, rs5985, rs1801133, rs 1800790, rs2232354, rs9574, rs5985, rs 1800595, rs 1799963, rs2232698, SERPINA10 Chr. 14: 93824396 R, PROC Chr. 2: 127900253 Y, PROC Chr. 2: 127895484 R, PROC Chr. 2: 127902541 Y, PROS1 Chr. 3: 95080840 Y, PROS1 Chr. 3: 95086439 R, F11 Chr. 4: 187429867 Y, F11 Chr. 4: 187438406 Y, FGA Chr. 4: 155730115 R, FGA Chr. 4: 155727040 R, THBD Chr. 20: 22976686 K, rs5907, FGB Chr. 4: 155706587 R, TFPI Chr. 2: 188057188 Y, PLG Chr. 6: 161079615 R, FGG Chr. 4: 155747369 W, SERPINC1 Chr. 1: 172150331 Y, and SERPINC1 Chr. 1: 172139799 S.

40. The method of claim 1, wherein said individual selects said two or more phenotypes.

41. The method of claim 1, wherein said set of genetic variants was identified using a high density DNA microarray.

42. The method of claim 1, wherein said set of genetic variants was identified by sequencing genomic DNA from said individual.

43. The method of claim 1, wherein said individual is a military trainee.

44. The method of claim 1, wherein said individual is a member of the military.

45. The method of claim 1, wherein said individual is a law enforcement officer.

46. The method of claim 1, wherein the results of said determination are used to rank applicants for service in the military or a law enforcement agency.

47. The method of claim 1, wherein said individual tests positive for a sensitivity or adverse reactions from small pox vaccination phenotype, said method further comprising disqualifying said individual from small pox vaccination or from duties likely to expose said individual to smallpox.

48. The method of claim 1, wherein said individual tests positive for a psychiatric illness phenotype, said method further comprising monitoring said individual for signs of psychiatric illness.

49. The method of claim 1, wherein said individual tests positive for a psychiatric illness phenotype, said method further comprising disqualifying said individual for service in the military or a law enforcement agency.

50. A suitability-for-military-service set of probes, wherein said set comprises probes, wherein each of said probes is specifically selected to detect a genetic variant correlated with a suitability-for-military-service-related safety phenotype.

51. The suitability-for-military-service-related set of probes of claim 50, wherein said set detects at least two phenotypes listed in the following figures: Military and Armed Forces Panel Alpha (FIG. 16), or Military and Armed Forces Panel Beta (FIG. 17).

52. The suitability-for-military-service-related set of probes of claim 50, wherein said set comprises at least two probes, and each of said at least two probes detects a different genetic variant, and wherein each of said different genetic variants is correlated to the same phenotype.

53. A method of determining the predisposition or carrier status of an individual for two or more Law Enforcement phenotypes related to comprising:
(a) identifying by nucleic acid array or sequencing apparatus a set of genetic variants in an individual, wherein each of said genetic variants is correlated with a Law Enforcement phenotype;
(b) using a computer to determine the predisposition or carrier status of said individual for at least two phenotypes, wherein said predisposition or carrier status is based on said set of genetic variants;
(c) providing a report of said predisposition or carrier status to said individual, to a health care provider of said individual, law enforcement official, forensic investigator, or to a third party; and, optionally,
(d) combining the predisposition or carrier status of said individual for said at least two phenotypes into a Law Enforcement score, wherein said score is reported to said individual, to a health care provider of said individual, or to a third party.

54. The method of claim 53, wherein said at least two phenotypes comprise an initial phenotype and a reflex phenotype, wherein said reflex phenotype is a phenotype that is not the initial phenotype, and wherein the reporting of the predisposition or carrier status of said individual for the reflex phenotype depends on the outcome of said determination of predisposition or carrier status of said individual for the first phenotype.

55. The method of claim 53, wherein said at least two phenotypes are at least two phenotypes listed in one or more of the following figure: Law Enforcement Panel (FIG. 18).

56. The method of claim 53, wherein said at least two phenotypes comprises at least five phenotypes.

57. The method of claim 53, wherein said at least two phenotypes comprise:
(a) at least one phenotype that follows monogenic inheritance; and
(b) at least one phenotype that follows multifactorial or polygenic inheritance.

58. The method of claim 53, wherein said at least two phenotypes comprises at least two of the following phenotypes: universal identifier or identity testing; blood group; physical traits; linear and/or ancestry information; height and/or weight; personality traits; psychiatric illness; age; cardiac arrhythmia or cardiac conduction abnormality; hypertrophic cardiomyopathy; thrombophilia or thromboembolic disease; stressful life events; visual acuity; level of aggression in behavior/personality; tendency to experience unprovoked anger; and mental vulnerability to social stressors and chronic disease.

59. The method of claim 54, wherein said reflex phenotype is reported when said individual has an increased predisposition or carrier status for said initial phenotype.

60. The method of claim 54, wherein said reflex phenotype is reported when said individual has a decreased predisposition or carrier status for said initial phenotype.

61. The method of claim 54, wherein said reflex phenotype is not reported if the individual has neither a decreased nor increased predisposition nor carrier status for said initial phenotype.

62. The method of claim 54, wherein said reflex phenotype is reported concurrently with said initial phenotype.

63. The method of claim 54, wherein said reflex phenotype is reported subsequently to said initial phenotype.

64. The method of claim 54, wherein the determination of the predisposition or carrier status of the individual for said reflex phenotype is determined subsequently to the determination of the predisposition or carrier status of the individual for said initial phenotype.

65. The method of claim 54, wherein said reflex phenotype is a disease that is positively correlated with said initial phenotype.

66. The method of claim 54, wherein said initial phenotype is a disease and said reflex phenotype is a symptom of said disease.

67. The method of claim 54, wherein said initial phenotype is a disease or disorder and reflex phenotype is a side effect of, or response to, a treatment for said initial phenotype.

68. The method of claim 54, wherein said initial phenotype is height or weight, and said reflex phenotype is one or more selected from the group consisting of: response of stature to human growth hormone; diabetes mellitus, type II; amount of effort needed to lose weight; dyslipidemia and/or lipid levels with increased BMI and/or obesity; change in body fat and/or lipid levels with specific diets and/or with exercise; exercise tolerance and/or optimal exercise regimen and/or athletic training regimen for weight management.

69. The method of claim 54, wherein said initial phenotype is psychiatric illness, and said reflex phenotype is one or more selected from the group consisting of: treatment-emergent suicidality during treatment with antidepressants; effectiveness and/or sensitivity and/or response to medications used to treat depression; response rates to standard treatment for late-life depression; aggressiveness or homicidal behavior with schizophrenia; severity or symptomology of schizophrenia; aggressiveness or homicidal behavior with schizophrenia; dose and/or choice and/or effectiveness and/or sensitivity and/or response and/or adverse reactions to mood stabilizers and/or antipsychotic medications; cognitive performance with bipolar disorder; antipsychotic medication induced parkinsonism; lithium response in mania and/or bipolar disorder.

70. The method of claim 54, wherein said initial phenotype is cardiac arrhythmia or cardiac conduction abnormality, and said reflex phenotype is one or more selected from the group consisting of: drug-induced torsade de pointes; drug-induced long QT syndrome; suitability of antiarrhythmogenic medication; digoxin suitability; age of onset of atrial fibrillation; QTc length, severity, symptoms, and prognosis with long QT syndrome.

71. The method of claim 54, wherein said initial phenotype is hypertrophic cardiomyopathy, and said reflex phenotype is heart wall thickness with cardiomyopathy.

72. The method of claim 54, wherein said initial phenotype is thrombophilia or a thromboembolic disorder, and said reflex phenotype is one or more selected from the group consisting of: warfarin suitability; and suitability of anti-thrombotic medications or NSAIDs.

73. The method of claim 54, wherein said initial phenotype is depression or seasonal affective disorder and said reflex phenotype is one or more selected from the group consisting of: suitability of medications used to treat depression; treatment-emergent suicidality during treatment with antidepressants; response to treatment for depression; and suitability of medication for treatment of anxiety.

74. The method of claim 54, wherein said initial phenotype is stressful life events causing depressive symptoms diagnosable depression or anxiety, and said reflex phenotype is one or more selected from the group consisting of: suitability of medications used to treat depression; treatment-emergent suicidality during treatment with antidepressants; and effectiveness and choice of medication for treatment for anxiety.

75. The method of claim 53, wherein said predisposition or carrier status is determined from at least two genetic variants.

76. The method of claim 75, wherein said at least two genetic variants are correlated with the same phenotype.

77. The method of claim 75, wherein said predisposition or carrier status is determined for height or weight and at least one of said genetic variants is selected from the group consisting of, or in linkage disequilibrium with, at least one genetic variant selected from the group consisting of: rs6060369, rs6830062, rs1867138, rs724016, rs7846385, rs 1492820, rs 10946808, rs314277, rs4896582, rs2040494, rs9650315, rs 1042725, rs8007661, rs2562784, rs12986413, rs6060369, rs6440003, rs2282978, rs6060373, rs1390401, rs3116602, rs6686842, rs10906982, rs7901695, rs6724465, rs10935120, rs8041863, rs4794665, rs757608, rs4800148, rs967417, rs16896068, rs4549631, rs3791675, rs2814993, rs10512248, rs12735613, rs11107116, rs6854783, rs8099594, rs11205277, rs678962, rs2274432, rs3791679, rs6763931, rs6842303, rs1812175, rs12198986, rs2844479, rs3130050, rs185819, rs1776897, rs4713858, rs3748069, rs798544, rs11765954, rs10498015, rs10958476, rs4743034, rs8756, rs7153027, rs4533267, rs3760318, rs324420, rs9930506, rs4740294, rs2241766, rs9939609, rs1801260, rs2293855, rs2272382, rs745229, rs4129733, rs17782313, rs1528133, rs7799039, rs1801282, rs7566605, rs4632532, rs7561317, rs182052, rs1042713, rs2889849, rs10498015, rs1421085, rs1528133, rs7034356, rs8050136, rs1455832, rs2774279, rs968671, rs2241766, rs4818, rs7138803, and rs4680.

78. The method of claim 75, wherein said predisposition or carrier status is determined for suicidality and at least one of said genetic variants is selected from the group consisting of, or in linkage disequilibrium with, at least one genetic variant selected from the group consisting of: rs1386494, rs25531, rs12936511, rs6265, rs4792887, and rs4675690.

79. The method of claim 75, wherein said predisposition or carrier status is determined for bipolar disorder and at least one of said genetic variants is selected from the group consisting of, or in linkage disequilibrium with, at least one genetic variant selected from the group consisting of: rs1298865, rs942518, rs12899449, rs17110563, rs25531, rs1006737, rs12899449, rs41261045, rs10994336, rs4511, rs9462082, rs4680, rs6265, rs2230912, rs133845, ADRBK2 Chr. 22: 24290897 delG, rs6986303, rs138784, rs 1170191, rs821633, rs 11089599, rs1344706, rs11568190, rs2391191, rs3918346, rs2637777, rs7680321, rs2304865, rs4979416, rs10994336, rs1485171, rs12899449, and rs10937823.

80. The method of claim 75, wherein said predisposition or carrier status is determined for atrial fibrillation and at least one of said genetic variants is selected from the group consisting of, or in linkage disequilibrium with, at least one genetic variant selected from the group consisting of: KCNJ2 Chr. 17: 65683052 R, rs2200733, rs10033464, rs 13143308, KCNJ1 Chr. 17: 65683052 R, KCNQ1 Chr. 11: 2505765 R, KCNQ1 Chr. 11: 2505768 R, and KCNE2 Chr. 21: 34664726 Y.

81. The method of claim 75, wherein said predisposition or carrier status is determined for hypertrophic cardiomyopathy and at least one of said genetic variants is selected from the group consisting of, or in linkage disequilibrium with, at least one genetic variant selected from the group consisting of: rs28933099, rs3218713, rs2856655, MTTH Mito: 12192 R, MTTL1 Mito: 3303 Y, MYL2 Chr: 12: 109841320 R, MYH7 Chr. 14: 22968327 R, MYH7 Chr. 14: 22968054 Y, and MYBPC3 Chr. 11: 47320705 S.

82. The method of claim 75, wherein said predisposition or carrier status is determined for thrombophilia and at least one of said genetic variants is selected from the group consisting of, or in linkage disequilibrium with, at least one genetic variant selected from the group consisting of: rs6025, rs6046, rs5985, rs1801133, rs1800790, rs2232354, rs9574, rs5985, rs1800595, rs1799963, rs2232698, SERPINA10 Chr. 14: 93824396 R, PROC Chr. 2: 127900253 Y, PROC Chr. 2: 127895484 R, PROC Chr. 2: 127902541 Y, PROS1 Chr. 3: 95080840 Y, PROS1 Chr. 3: 95086439 R, F11 Chr. 4: 187429867 Y, F11 Chr. 4: 187438406 Y, FGA Chr. 4: 155730115 R, FGA Chr. 4: 155727040 R, THBD Chr. 20: 22976686 K, rs5907, FGB Chr. 4: 155706587 R, TFPI Chr. 2: 188057188 Y, PLG Chr. 6: 161079615 R, FGG Chr. 4: 155747369 W, SERPINC1 Chr. 1: 172150331 Y, and SERPINC1 Chr. 1: 172139799 S.

83. The method of claim 75, wherein said predisposition or carrier status is determined for Exfoliation Glaucoma and at least one of said genetic variants is selected from the group consisting of, or in linkage disequilibrium with, at least one genetic variant selected from the group consisting of: rs1801133, rs1048661, and rs3825942.

84. The method of claim 53, wherein said individual selects said two or more phenotypes.

85. The method of claim 53, wherein said set of genetic variants was identified using a high density DNA microarray.

86. The method of claim 53, wherein said set of genetic variants was identified by sequencing genomic DNA from said individual.

87. The method of claim 53, wherein said individual is a patient.

88. The method of claim 53, wherein said individual is a suffering from an unknown disease or condition.

89. The method of claim 53, wherein said individual is an organ, cell, or tissue transplant candidate.

90. The method of claim 53, wherein said individual has died of unknown causes.

91. A Law Enforcement set of probes, wherein said set comprises probes, wherein each of said probes is specifically selected to detect a genetic variant correlated with a Law Enforcement phenotype.

92. The Law Enforcement set of probes of claim 91, wherein said set detects at least two phenotypes listed in the following figure: Law Enforcement Panel (FIG. 18).

93. The Law Enforcement set of probes of claim 91, wherein said set comprises at least two probes, and each of said at least two probes detects a different genetic variant, and wherein each of said different genetic variants is correlated to the same phenotype.
